(19) United States
(12) Patent Application Publication
(10) Pub. No.: US 2024/0287605 A1
(43) Pub. Date: Aug. 29, 2024

(54) EPIGENETIC CLOCKS

(71) Applicant: The Regents of the University of California, Oakland, CA (US)

(72) Inventor: Stefan Horvath, Los Angeles, CA (US)

(73) Assignee: The Regents of the University of California, Oakland, CA (US)

(21) Appl. No.: 18/572,130

(22) PCT Filed: Jun. 24, 2022
(86) PCT No.: PCT/US22/34978

§ 371 Date: Dec. 19, 2023

Related U.S. Application Data
(60) Provisional application No. 63/215,289, filed on Jun. 25, 2021.

(51) Int. Cl. C12Q 1/6876

(52) U.S. Cl. CPC C12Q 1/6876; C12Q 2600/154

(57) ABSTRACT

DNA methylation profiles have been used to develop biomarkers of aging known as epigenetic clocks, which predict chronological age with remarkable accuracy and show promise for inferring health status as an indicator of biological age. Epigenetic clocks were first built to monitor human aging but the principles underpinning them appear to be evolutionarily conserved. Here we describe reliable and highly accurate epigenetic clocks shown to apply to humans and other mammals.

### CROSS-REFERENCE TO RELATED APPLICATIONS

This application claims the benefit under 35 U.S.C. Section 119(e) of co-pending and commonly-assigned U.S. Provisional Patent Application Ser. No. 63/215,289, filed on Jun. 25, 2021, and entitled “EPIGENETIC CLOCKS” which application is incorporated by reference herein.

### TECHNICAL FIELD

The invention relates to methods and materials for examining biological aging in mammals.

### BACKGROUND OF THE INVENTION

Studies in invertebrates (yeast, worm, flies) have led to a long list of pharmacological agents that promise to intervene in different aspects of the aging process including stress response mimetics, anti-inflammatory interventions, epigenetic modifiers, neuroprotective agents, hormone treatments. While our arsenal of potential anti-aging interventions is brimming with highly promising candidates that delay aging in model organisms, it remains to be seen whether these interventions delay aging in human and other mammalian cells. To facilitate effective in vitro and ex vivo studies, there is a need for biomarkers of aging. One potential biomarker that has gained significant interest in recent years is DNA methylation (DNAm). DNA methylation by the attachment of a methyl group to cytosines is one of the most widely studies epigenetic modifications, due to its implications in regulating gene expression across many biological processes. Chronological time has been shown to elicit predictable hypo- and hyper-methylation changes at many regions across the genome and several DNAm based biomarkers of aging have been developed. These epigenetic age estimators exhibit statistically significant associations with many age-related diseases and conditions.

In humans. DNA methylation levels can be used to accurately predict an individual's age, as well as age across tissues and cell types. DNA methylation-based biomarkers allow one to estimate the epigenetic age of an individual. For example, the pan tissue epigenetic clock, which is based on 353 dinucleotide markers, known as CpGs (-C-phosphate-G-), can be used to estimate the age of most human cell types, tissues, and organs (Horvath S. DNA methylation age of human tissues and cell types. Genome Biol. 2013; 14 (R115). The estimated age, referred to as “DNA methylation age” (DNAm age), correlates with chronological age when methylation is assessed in certain cell types, tissues, and organs.

The first human methylation chip (ILLUMINA INFINIUM 27K) was introduced over ten years ago. However, there are few platforms that focus on specific aspects of human aging and/or aging in non-human mammalian species. In view of this, there is a need for methods and materials useful for observing specific methylation profiles and associated phenomena that relate to aspects of aging in both humans and other mammalian species.

### SUMMARY OF THE INVENTION

The invention disclosed herein provides methods and materials designed to observe DNA methylation levels at selected sites within genomes of humans and other mammalian species. Using these methods and materials, embodiments of the invention provide a number of different biomarkers useful both for predicting the lifespan of humans and a number of other mammals, as well as assessing other physiological factors associated with aging. As discussed in detail below, embodiments of the invention observe methylation levels at a variety of selected sites within genomes of humans and other mammalian species in order to obtain information on a variety of physiological phenomena associated with aging such as life expectancy, mortality, and morbidity. Embodiments of the invention that focus on the prediction of mortality and morbidity in humans show that these DNAm based biomarkers are highly informative for a range of applications.

As discussed below, embodiments of the invention include methods for generating predictors of age-related phenomena in mammals, for example age and lifespan. In particular, it has been discovered that a number of CpG methylation sites in mammalian genomes are conserved across mammalian species & tissues. Building upon these discoveries, a number of methods that combine high accuracy age prediction with maximal biological and translational relevance have been developed. Embodiments of the invention can be used, for example, as predictors of “chronological age” or “epigenetic age” in various mammals. Embodiments of the invention also include methods for monitoring and tracking how aging process changes methylation patterns associated with the epigenetic aging of human and other mammalian cells under a wide variety of conditions. For example, embodiments of the invention include in vitro and in vivo methods for observing and monitoring the effects of one or more test agents or treatments on genomic methylation patterns associated with the epigenetic aging of human and other mammalian cells. In one illustrative example of such methods, an embodiment of the invention uses observations of changes in the disclosed methylation profiles that associated with of “epigenetic age” before and after exposure to an agent or other environmental condition that may modulate such age-related methylation profiles.

As shown in the examples, the DNA methylation profiles disclosed herein that are predictors of “actual/chronological age” and/or “epigenetic age” are accurate for multiple mammalian species. In this context, embodiments of the invention can be applied to individual species or groups of species for increased accuracy. Such embodiments of the invention include pan-tissue epigenetic clocks for humans and dogs and/or rats and/or mice. In certain embodiments of the invention, DNA is obtained from specific species, groups of species, tissues or groups of tissues (e.g., blood) for increased accuracy. In addition, embodiments of the invention are applied to specific species relationships for increased translational relevance (e.g., dogs and humans, rat and humans, mice and humans). Moreover, embodiments of the invention are designed to be applied to other complex age-related traits including predicted lifespan, maximum lifespan across species, average to time to death and the like.

The invention disclosed herein has a number of embodiments. Embodiments of the invention include, for example, methods for obtaining information associated with an age of a mammal, the method comprising: obtaining genomic DNA from the mammal; observing CpG methylation of the genomic DNA in a group of at least 40 methylation markers present in genomic polynucleotides having SEQ ID NO: 1-SEQ ID NO: 3880; and then correlating methylation observed in the methylation markers with an age of the mammal; so that information associated with an age of the mammal is obtained. In some embodiments of the invention, methylation of the genomic DNA is observed in a plurality of methylation markers present in in polynucleotides having SEQ ID NO: 1-SEQ ID NO: 956 such that the methylation markers observed are selected to be methylation markers whose methylation status is associated with an age in both humans and dogs. In some embodiments of the invention, methylation of the genomic DNA is observed in a plurality of methylation markers present in in polynucleotides having SEQ ID NO: 2220-SEQ ID NO: 3043 such that methylation markers observed are selected to be methylation markers whose methylation status is associated with an age in both humans and rats. In some embodiment of the invention, methylation of the genomic DNA is observed in a plurality of methylation markers present in in polynucleotides having SEQ ID NO: 1222-SEQ ID NO: 2219 such that methylation markers observed are selected to be methylation markers whose methylation status is associated with an age in both humans and mice. In some embodiments of the invention, methylation of the genomic DNA is observed in a plurality of methylation markers present in in polynucleotides having SEQ ID NO: 3044-SEQ ID NO: 3880 such that methylation markers observed are selected to be methylation markers whose methylation status is associated with an age in a plurality of mammalian species.

Certain embodiments comprise correlating methylation observed in the methylation markers with an epigenetic age and/or a chronological age of the mammal. In some embodiments of the invention, the method further compares an epigenetic age obtained for the sample with the chronological age of the mammal. Optionally, the method comprises determining an epigenetic age of the biological sample with a statistical prediction algorithm, comprising (a) obtaining a linear combination of the methylation marker levels, and (b) applying a transformation to the linear combination to determine an epigenetic age of the biological sample. In some embodiments of the invention, methylation is observed by a process comprising treatment of genomic DNA from the population of cells from the individual with bisulfite to transform unmethylated cytosines of CpG dinucleotides in the genomic DNA to uracil; and/or genomic DNA is obtained from fibroblasts, keratinocytes, buccal cells, endothelial cells, lymphoblastoid cells, and/or cells obtained from blood, skin, dermis, epidermis or saliva; and/or genomic DNA is hybridized to a complimentary sequence disposed on a microarray; and/or correlating observed methylation in the methylation markers comprises a regression analysis.

Embodiments of the invention also include methods for observing the effects of an environmental condition on genomic methylation associated epigenetic aging of mammalian cells, the methods comprising: (a) exposing mammalian cells to the environmental condition; (b) observing methylation status in at least 40 of the methylation markers present in polynucleotides having SEQ ID NO: 1-SEQ ID NO: 3880 in genomic DNA from the mammalian cells; and then (c) comparing the observations from (b) with observations of a methylation status at least 40 of the methylation markers present in in polynucleotides having SEQ ID NO: 1-SEQ ID NO: 3880 in genomic DNA from control mammalian cells not exposed to the environmental condition such that effects of the environmental condition on genomic methylation associated epigenetic aging in the mammalian cells is observed. In some embodiments of the invention, the plurality of the methylation markers observed are selected to be methylation markers whose methylation status is associated with age in both humans and dogs; and/or the cells are human and/or dog cells. In some embodiments of the invention, the plurality of the methylation markers observed are selected to be methylation markers whose methylation status is associated with age in both humans and rats; and/or the cells are human and/or rat cells. In some embodiments of the invention, the plurality of the methylation markers observed are selected to be methylation markers whose methylation status is associated with age in both humans and mice; and/or the cells are human and/or mouse cells. In some embodiments of the invention, a plurality of the methylation markers observed are selected to be methylation markers whose methylation status is associated with an age in a plurality of mammalian species and the cells are human cells.

In certain embodiments of the methods for observing the effects of an environmental condition on genomic methylation associated epigenetic aging of mammalian cells, methylation is observed by a process comprising treatment of genomic DNA from the population of cells from the individual with bisulfite to transform unmethylated cytosines of CpG dinucleotides in the genomic DNA to uracil; and/or genomic DNA is obtained from fibroblasts, keratinocytes, buccal cells, endothelial cells, lymphoblastoid cells, and/or cells obtained from blood, skin, dermis, epidermis or saliva; and/or genomic DNA is hybridized to a complimentary sequence disposed on a microarray; and/or correlating observed methylation in the methylation markers comprises a regression analysis. In some embodiments of the invention, the environmental condition comprises exposure to a composition of matter. In certain of these embodiments of the invention, the composition of matter is combined with mammalian cells for at least 1 day, at least 1 week or at least 1 month. Optionally, the composition of matter comprises a test agent having a molecular weight of <900 Da.

Embodiments of the invention further include a tangible computer-readable medium comprising computer-readable code that, when executed by a computer, causes the computer to perform operations comprising receiving information corresponding to a methylation status of a set of methylation markers in a biological sample, said methylation markers comprising methylation markers present in genomic polynucleotides having SEQ ID NO: 1-SEQ ID NO: 3880; and then determining an age of the biological sample by applying a statistical prediction algorithm to the measured methylation marker levels. Typically, the tangible computer-readable medium further comprises a computer-readable code that, when executed by a computer, causes the computer to perform one or more additional operations comprising: sending information corresponding to the methylation levels of the set of methylation markers in the biological sample to a tangible data storage device.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1A-1J: Data from cross-validation studies of epigenetic clocks for dogs. To arrive at unbiased estimates of the dog epigenetic clock we carried out two types of validation studies: FIG. 1(a) leave one out (LOO) cross validation and, FIG. 1(b) leave-one-breed-out cross validation (LOBO). FIG. 1(c, d) performance of the dog clock in FIG. 1(c) different human tissues and FIG. 1(d) human blood tissue. FIGS. 1(e,f,g,h) Species balanced cross validation (LOFO10Balance) analysis of human dog clocks for FIGS. 1(e,f) chronological age and FIGS. 1(g,h) relative age. Human dog clock for chronological age applied to FIG. 1(e) both species and FIG. 1(f) dogs only. Human dog clock for relative age applied to FIG. 1(g) both species, FIG. 1(h) dogs only. FIGS. 1(i,j) LOBO cross validation of the human dog clock for FIG. 1(i) chronological age and FIG. 1(j) relative age in blood samples from dogs. Species balanced cross validation (LOFO10Balance) was implemented in the following steps. First, we partitioned both the combined human/dog dataset into 10 evenly sized folds, where each fold has the same proportion and human and dog samples (referred to as “balanced” folds). We then iterate through each fold, training on the other nine folds, and applied the model to the target fold. Each panel reports the sample size, correlation coefficient, median absolute error (MAE).

FIGS. 2A-2F. Epigenetic clocks for prediction average time to death. FIG. 2(a) Leave-one-breed-out (LOBO) estimates of DNA methylation (DNAm) average time to death (y-axis, in units of years) versus average time to death (x-axis in units of years). For each dog, the average time to death was defined as difference between the upper limit of the respective breed lifespan (Lifespan.HighClubBreeder) and chronological age. FIG. 2(b) LOBO DNAm average time to death adjusted for age (y-axis) versus lifespan (x-axis, in units of year). FIG. 2(c) LOBO DNAm average time to death adjusted for age (y-axis) versus adult weight (x-axis). FIG. 2(d) LOBO DNAm average time to death (y-axis) versus chronological age (x-axis). The association between LOBO DNAm average time to death adjusted for age and the lifespan remains significant (p=4.4×10-3) even after adjusting for average adult weight in a multivariate regression model. FIG. 2(e) Phylogenetically Independent (Indep.) contrast (PIC) generated LOBO DNAm average time to death adjusted for age (y-axis, at breed level) versus PIC generated lifespan (x-axis, at breed level). FIG. 2(f) PIC generated LOBO DNAm average time to death adjusted for age (y-axis, at breed level) versus PIC generated adult weight (x-axis, at breed level). At each panel, we report Pearson correlation estimate. Each individual dog was marked in breed index as listed in the legend of FIG. 1.

FIGS. 3A-3F. Epigenome wide association analysis of chronological age, average breed lifespan, and average breed weight of dog blood. FIG. 3(a) Manhattan plots of the EWAS results. Coordinates are estimated based on the alignment of the mammalian array probes to the CanFam_GreatDane.UMICH_Zoey_3.1.100 genome assembly. The direction of associations with p<10-3 (red dotted line) is highlighted by red (hypermethylated) and blue (hypomethylated) colors. Top 15 CpGs was labeled by the neighboring genes. FIG. 3(b) Location of top CpGs in each tissue relative to the closest transcriptional start site. The grey color in the last panel represents the location of 31,911 on the mammalian array that mapped to the genome of the Great Dane. Top CpGs were selected at p<10-3. For age, top 1,000 CpGs were selected positive or negative direction. The number of selected CpGs: Age, 1,000; lifespan; 162; weight, 406. FIG. 3(c) Boxplot of DNAm aging in CpGs located in island or outside. Labels indicate neighboring genes of the top four CpGs in each analysis. ***p<10-4. FIG. 3(d) Venn diagram showing the overlap of top CpGs associated with chronological age, breed lifespan, and breed weight. The (+) and (−) signs in the table show the direction of association with each variable. FIG. 3(c) Sector plot of EWAS of dog breed lifespan and weight. Red dotted line: p<10-3; blue dotted line: p>0.05; Red dots: shared CpGs; black dots: lifespan- or weight-specific changes. FIG. 3(f) Enrichment analysis EWAS-GWAS associated genes. The heat map represents the significant results from the genomic-region based enrichment analysis between (1) the top 5% genomic regions involve in GWAS of complex traits-associated genes and (2) up to the top 1,000 hypermethylated/hypomethylated CpGs from EWAS of lifespan, adult weight, lifespan adjusted adult weight at breed level, and age at individual dog level, respectively. Cells are colored in grey if nominal P>0.05. The heat map color gradient is based on −log 10 (hypergeometric P value). We list the GWAS study (x-axis) if that at least one enrichment P value (columns) is significant at a nominal significance level of 3.0×10-3. The v-axis lists GWAS index number, trait name. The color band next to the trait encodes the GWAS category. Abbreviations: All=All ancestries, WHR=waist to hip ratio, EUR=European ancestry, FrD=frontal temporal dementia.

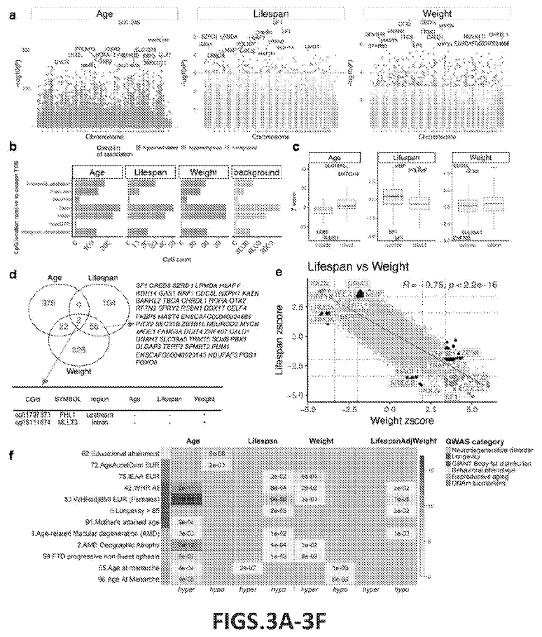

FIGS. 4A-4B. Genes having both genetic variants and methylation patterns that relate to dog breed weight or lifespan. Scatter plots of DNAm changes with strong correlation with lifespan FIG. 4(a), or adult weight FIG. 4(b) in genes selected by GWAS of weight in dog breeds 3, 16. The title of each panel reports the CpG and an adjacent gene. The blue text inside the panel reports the Pearson correlation coefficient and the p value.

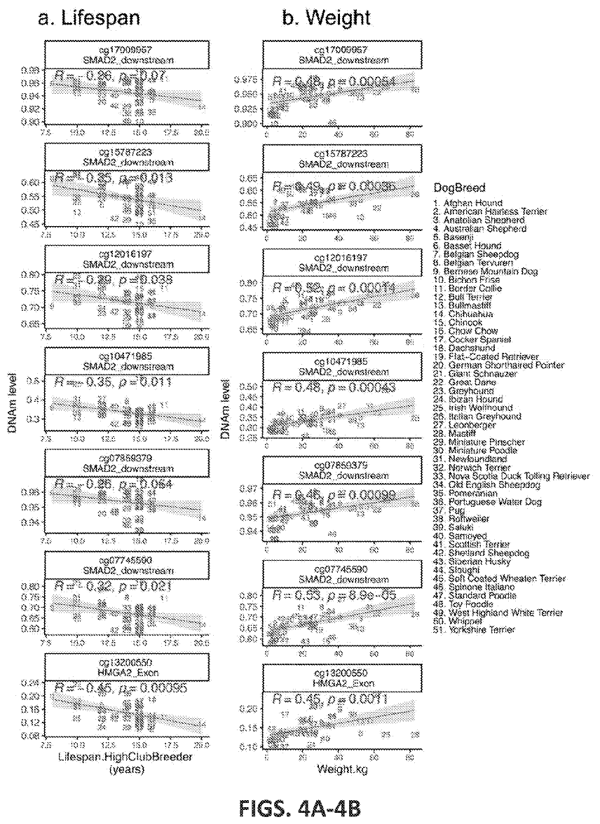

FIGS. 5A-5H. Cross-validation studies of epigenetic clocks for rat. FIGS. 5(A-D) Four epigenetic clocks that were trained on rat tissues only. FIGS. 5(E-H) Results for 2 clocks that were trained on both human and rat tissues. Leave-one-sample-out estimate of DNA methylation age (y-axis, in units of years) versus chronological age for FIG. 5(A) Rat pan-tissue, FIG. 5(B) Rat brain, FIG. 5(C) Rat blood, and FIG. 5(D) Rat liver clock. Dots are colored by FIG. 5(A) tissue type or FIG. 5(B) brain region. FIG. 5(E) and FIG. 5(F) “Human-rat” clock estimate of absolute age. FIGS. 5(G. H) Human-rat clock estimate of relative age, which is the ratio of chronological age to maximum lifespan in the respective species. Ten-fold cross-validation estimates of age (y-axis, in years) in FIGS. 5(E, G) Human (green) and rat (orange) samples and FIGS. 5(F, H) rat samples only (colored by tissue type). Each panel reports the sample size, correlation coefficient, median absolute error (MAE).

FIGS. 6A-6G. Meta-analysis of chronological age across species and tissues. FIG. 6(a), Meta-analysis p-value (−log base 10 transformed) versus chromosomal location (x-axis) according to human genome assembly 38 (Hg38). The upper and lower panels of the Manhattan plot depict CpGs that gain/lose methylation with age. CpGs colored in red and blue exhibit highly significant (P<10-200) positive and negative age correlations, respectively. The most significant CpG (cg12841266, P=9.3×10-913) is located in exon 2 of the LHFPL4 gene in humans and most other mammalian species, followed by cg11084334 (P=1.3×10-827). These two CpGs and cg097720 (P=4.3×10-725) located in the paralog gene LHFPL3 are marked in purple diamond points. Scatter plots of cg12841266 (in x-axis) versus chronological age in FIG. 6(b), mini pigs (Sus scrofa minusculus), FIG. 6(c), Oldfield mouse (Peromyscus polionotus), and FIG. 6(d), vervet monkey (Chlorocebus aethiops sabacus), respectively. Tissue samples are labelled by the mammalian species index and colored by tissue type. Panels e-g: annotations of the top 1000 hypermethylated and hypomethylated CpGs listed in the EWAS meta-analysis across all (results in panel a), brain, blood, liver, and skin tissues, respectively. FIG. 6(e), the Venn diagram displays the overlap of age-associated CpGs across different organs, based on EWAS of the top 1000 hypermethylated/hypomethylated CpGs. We list all 36 genes that are proximal to the 54 age-associated CpGs common across all organs in the Venn diagram. FIG. 6(f), the bar plots depicts the associations of the EWAS results (meta Z scores) with CpG islands (inside/outside) in different tissue types. We list top genes for each bar. FIG. 6(g), Selected results from GREAT enrichment analysis. The color gradient is based on −log 10 (hypergeometric P value). The size of the points reflects the number of common genes.

FIGS. 7A-7F. Naïve universal clock for log-transformed age. FIG. 7(a, b), Chronological age (x-axis) versus DNAmAge estimated using a, leave-one-fraction-out (LOFO) b, leave-one-species-out (LOSO) analysis. The grey and black dashed lines correspond to the diagonal line (y=x) and the regression line, respectively. Each dot (tissue sample) is labelled by the mammalian species index (legend). The species index corresponds to the phylogenetic order, e.g. 1=primates, 2=elephants (Proboscidea), 3=cetaceans etc. The number after the decimal point denotes the individual species within the phylogenetic order. Points are colored according to designated tissue color. The heading of each panel reports the Pearson correlation (cor) across all samples. The med.Cor (or med.MAE) is the median across species that contain 15 or more samples. FIG. 7(c-f), Delta age denotes the difference between the LOSO estimate of DNAm age and chronological age. The scatter plots depict mean delta age per species (y-axis) versus FIG. 7(c), maximum lifespan observed in the species, FIG. 7(d), average age at sexual maturity FIG. 7(e), gestational time (in units of years), and FIG. 7(f), (log-transformed) average adult weight in units of grams.

FIGS. 8A-8L. Universal clocks for transformed age across mammals. The figure displays universal clock 2 (Clock 2) estimates of relative age, universal clock 3 (Clock 3) estimates of log-linear transformation of age and marsupial clock (Marsupial Clock) estimates of relative age of eutherian and marsupial samples respectively. Relative age estimation incorporates maximum lifespan and gestational age, and assumes values between 0 and 1. Log-linear age is formulated with age at sexual maturity and gestational time. The DNAm estimates of age (y axes) of FIGS. 8(a) and (b) are transformation of relative age (Clock 2 and Marsupial Clock) or log-linear age (Clock 3), into units of years. FIG. 8(a-f), Age estimated via leave-one-fraction-out (LOFO) cross-validation for Clock 2 in FIG. 8(a,d), Clock 3 in FIG. 8(b,e) and Marsupial Clock in FIG. 8(c,f). FIG. 8(g-i), Age estimated via LOFO cross-validation in Clock 2. FIG. 8(j-l), age estimated via leave-one-species-out (LOSO) cross-validation for Clock 2. We report Pearson correlation coefficient estimates. Median correlation (med.Cor) and median of median absolute error (med.MAE) are calculated across species in FIG. 8(a-f) or across species-tissue in FIG. 8(g-i). Each sample is labelled by mammalian species index and marked by tissue color (FIG. 7).

FIGS. 9A-9P. Universal clock for relative age applied to specific tissues. The specific tissue or cell type is reported in the title of each panel. DNA methylation based estimates of relative age (y-axis) versus actual relative age (x-axis). Each dot presents a tissue sample colored by tissue and labelled by mammalian species index. The analysis is restricted to tissues with at least 15 samples available. Leave-one-folder-out cross-validation (LOFO) was used to arrive at unbiased estimates of predictive accuracy measures: median absolute error (MAE) and age correlation based on relative age. “Cor” denotes the Pearson correlation coefficient based on all available samples. “med.Cor” denotes the median values across all species for which at least 15 samples were available. Title is marked in blue if a tissue type was collected from a single species.

FIGS. 10A-10D. Human-mouse epigenetic clock. DNA methylation estimates of FIG. 10(a) relative age and FIG. 10(b) chronological age in samples from mice and humans. The y-axis reports cross validation estimates of the DNAm based age estimator. The x-axis reports the actual values. Points are labelled by species number (9.1=mouse and 1.1=human) and colored by tissue/cell type. Analysis restricted to FIG. 10(c) human samples and FIG. 10(d) mouse samples. Each panel reports the Pearson correlation and the median absolute error (MAE) and median values across different tissue types. We analyzed N=1982 mouse tissues and N=1211 human tissues.

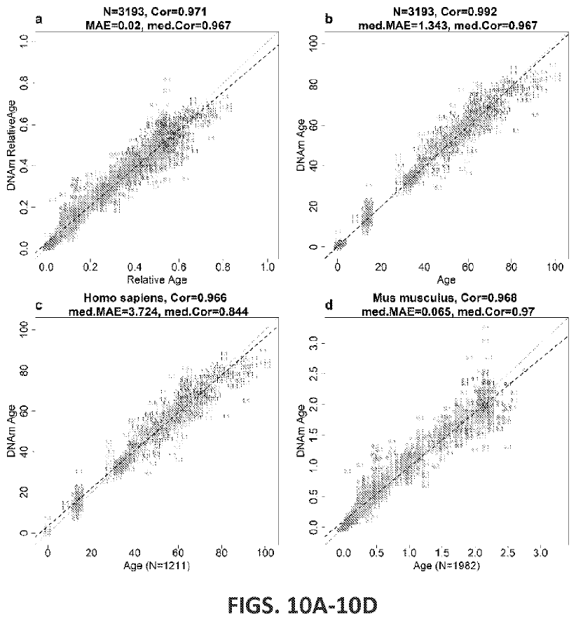

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

All publications mentioned herein are incorporated herein by reference to disclose and describe aspects, methods and/or materials in connection with the cited publications. For example, Lu et al., Aging (Albany NY). 2019 Jan. 21; 11(2):303-327. doi: 10.18632/aging 101684; PCT Patent Application No.: PCT/US2019/034829, U.S. Patent Publication 20150259742, U.S. patent application Ser. No. 15/025,185, titled “METHOD TO ESTIMATE THE AGE OF TISSUES AND CELL TYPES BASED ON EPIGENETIC MARKERS”, filed by Stefan Horvath; U.S. patent application Ser. No. 14/119,145, titled “METHOD TO ESTIMATE AGE OF INDIVIDUAL BASED ON EPIGENETIC MARKERS IN BIOLOGICAL SAMPLE”, filed by Eric Villain et al.; and Hannum et al. “Genome-Wide Methylation Profiles Reveal Quantitative Views Of Human Aging Rates.” Molecular Cell. 2013; 49(2):359-367 and patent US2015/0259742, are incorporated by reference in their entirety herein.

The invention disclosed herein provides novel and powerful biomarker predictors of physiological factors such as chronological age, relative age, life expectancy, mortality, and morbidity based on DNA methylation levels. Our discoveries surrounding the prediction of mortality and morbidity show that the DNAm based biomarkers disclosed herein are highly robust and informative for a range of applications. Embodiments of the DNAm based biomarkers disclosed herein can provide complementary information that enhances and supplements traditional biomarker assessments that are widely used in clinical applications. For example, embodiments of the invention can be used to directly predict/prognosticate mortality, and further information relating to a host of age-related conditions such as cardiovascular disease, cancer risk, progression in neurodegeneration, and various measures of frailty.

Embodiments of the invention include a number of different biomarker “clocks” useful for observing physiological factors such as chronological age, epigenetic age, relative age, life expectancy, mortality, and morbidity based on DNA methylation levels/profiles. The term “chronological age” refers to the actual age (e.g., in years) of the individual from whom a sample is obtained. The term “epigenetic age” is the age you are biologically. In this context, “epigenetic age” simply refers to the apparent “age” of an individual resulting from the interaction of its genotype with the environment of the individual from whom a sample is obtained. In this way and unlike chronological age, epigenetic age considers environmental factors that modulate human aging (e.g. environmental factors that can make folks “old before their time”). For example, by calendar years, you may have a chronological age of 50 years old, but your epigenetic age might be ten years, or any number of years, younger or older.

Embodiments of the invention are directed to methods of obtaining information on—factors associated with aging in mammals. In certain embodiments of the invention, these methods comprise the steps of: obtaining genomic DNA from the mammal; observing methylation of the genomic DNA in a group of at least 40 methylation markers found in a plurality of methylation markers present in in polynucleotides having SEQ ID NO: 1-SEQ ID NO: 3880; and then correlating observed methylation in the methylation markers with an age of a mammal (e.g. chronological age, epigenetic age and the like) such that information on factors associated with aging in mammals is obtained.

In certain embodiments of the invention, a plurality of the methylation markers observed are selected to be methylation markers whose methylation status is associated with a chronological and/or epigenetic age in both humans and dogs; and/or the methods are used to obtain information on factors associated with an age of a mammal such as a predication of chronological age, relative/epigenetic age or average time-to-death in humans and/or dogs. In certain embodiments of the invention; a plurality of the methylation markers observed are selected to be methylation markers whose methylation status is associated with a chronological and/or epigenetic age in both humans and rats; and/or a physiological factor associated with an age of a mammal comprises a predication of: chronological age or relative age or average time-to-death in humans and/or rats. In certain embodiments of the invention, a plurality of the methylation markers observed are selected to be methylation markers whose methylation status is associated with age in both humans and mice; and/or a physiological factor associated with an age of a mammal comprises a predication of: chronological age or relative age or average time-to-death in humans and/or mice.

In certain embodiments of the invention, methylation of the genomic DNA is observed in a plurality of the methylation markers selected to be methylation markers whose methylation status is universally associated with age in mammals; and/or a physiological factor associated with an age of a mammal comprises a predication of: chronological age or relative age or average time-to-death in mammals. In certain embodiments of the invention, a plurality of the methylation markers observed are selected to be methylation markers whose methylation status is associated with maximum lifespan in in humans and other mammals; and/or a physiological factor associated with an age of a mammal comprises a predication of: maximum lifespan in humans and other mammals.

Typically in the above-noted methods, methylation is observed by a process comprising treatment of genomic DNA from the population of cells from the individual with bisulfite to transform unmethylated cytosines of CpG dinucleotides in the genomic DNA to uracil; genomic DNA is obtained from fibroblasts, keratinocytes, buccal cells, endothelial cells, lymphoblastoid cells, and/or cells obtained from blood, skin, dermis, epidermis or saliva; genomic DNA is hybridized to a complimentary sequence disposed on a microarray; and/or correlating observed methylation in the methylation markers comprises a regression analysis.

Embodiments of the invention also include methods for observing the effects of an environmental condition on genomic methylation associated epigenetic aging of mammalian cells, the methods comprising: (a) exposing mammalian cells to the environmental condition; (b) observing methylation status in at least 40 of the methylation markers present in polynucleotides having SEQ ID NO: 1-SEQ ID NO: 3880 in genomic DNA from the mammalian cells; and then (c) comparing the observations from (b) with observations of a methylation status at least 40 of the methylation markers present in in polynucleotides having SEQ ID NO: 1-SEQ ID NO: 3880 in genomic DNA from control mammalian cells not exposed to the environmental condition such that effects of the environmental condition on genomic methylation associated epigenetic aging in the mammalian cells is observed. In some embodiments of the invention, the plurality of the methylation markers observed are selected to be methylation markers whose methylation status is associated with age in both humans and dogs; and/or the cells are human and/or dog cells. In some embodiments of the invention, the plurality of the methylation markers observed are selected to be methylation markers whose methylation status is associated with age in both humans and rats; and/or the cells are human and/or rat cells. In some embodiments of the invention, the plurality of the methylation markers observed are selected to be methylation markers whose methylation status is associated with age in both humans and mice; and/or the cells are human and/or mouse cells. In some embodiments of the invention, a plurality of the methylation markers observed are selected to be methylation markers whose methylation status is associated with an age in a plurality of mammalian species and the cells are human cells.

Certain embodiments of the invention include methods of observing the effects of one or more test agents on genomic methylation associated epigenetic aging of human and/or dog, rat or mouse cells both in vitro and in vivo. Typically, these methods comprise combining the test agent(s) with the cells (e.g. for specified period of time such as at least 1-7 days, 1-3 weeks, 1-6 months or the like), and then observing methylation status in at least at least 40 of the methylation markers disclosed herein in genomic DNA from the cells, and then comparing the observations from (b) with observations of the methylation status in at least at least 40 of the methylation markers disclosed herein in genomic DNA from control cells not exposed to the test agent such that effects of the test agent on genomic methylation associated epigenetic aging in the cells is observed. Optionally in these methods, a plurality of test agents are combined with the mammalian cells in vitro. In illustrative embodiments of the invention, the test agent is a polypeptide, a polynucleotide or a compound having a molecular weight less than 3,000, 2,000, 1,000 or 500 g/mol.

In certain embodiments of the methods of observing the effects of one or more test agents on genomic methylation associated epigenetic aging, the plurality of the methylation markers observed are selected to be methylation markers whose methylation status is associated with age in both humans and dogs; and/or the cells are human and/or dog cells. In certain embodiments, the plurality of the methylation markers observed are selected to be methylation markers whose methylation status is associated with age in both humans and rats; and/or the cells are human and/or rat cells. In certain embodiments, a plurality of the methylation markers observed are selected to be methylation markers whose methylation status is universally associated with age in mammals and/or the cells are human, mouse and/or rat cells. In certain embodiments, a plurality of the methylation markers observed are selected to be methylation markers whose methylation status is universally associated with age in mammals and/or the cells are human, mouse and/or rat cells. In certain embodiments, a plurality of the methylation markers observed are selected to be methylation markers whose methylation status is associated with maximum lifespan in in humans and other mammals; and/or the cells are human and/or mouse cells.

In certain embodiments of the methods of observing the effects of one or more test agents on genomic methylation associated epigenetic aging, methylation is observed by a process comprising treatment of genomic DNA from the population of cells from the individual with bisulfite to transform unmethylated cytosines of CpG dinucleotides in the genomic DNA to uracil; genomic DNA is obtained from fibroblasts, keratinocytes, buccal cells, endothelial cells, lymphoblastoid cells, and/or cells obtained from blood, skin, dermis, epidermis or saliva; genomic DNA is hybridized to a complimentary sequence disposed on a microarray; and/or correlating observed methylation in the methylation markers comprises a regression analysis.

### FURTHER ILLUSTRATIVE ASPECTS AND EMBODIMENTS OF THE INVENTION

Novel molecular biomarkers of aging, such as those termed “DNAm age”, “epigenetic age” or “apparent methylomic aging rate” allow one to prognosticate mortality, are interesting to gerontologists (aging researchers), epidemiologists, medical professionals, and medical underwriters for life insurances. Exclusively clinical biomarkers such as lipid levels, body mass index, blood pressures have a long and successful history in the life insurance industry. By contrast, molecular biomarkers of aging have rarely been used.

DNA methylation refers to chemical modifications of the DNA molecule. Technological platforms such as the Illumina Infinium microarray or DNA sequencing-based methods have been found to lead to highly robust and reproducible measurements of the DNA methylation levels of a person. There are more than 28 million CpG loci in the human genome. Consequently, certain loci are given unique identifiers such as those found in the Illumina CpG loci database (see, e.g. Technical Note: Epigenetics, CpG Loci Identification ILLUMINA Inc. 2010). These CG locus designation identifiers are used herein. In this context, one embodiment of the invention is a method of obtaining information useful to observe biomarkers associated with a phenotypic age of an individual by observing the methylation status of one or more of the methylation marker specific GC loci that are identified herein.

The term “epigenetic” as used herein means relating to, being, or involving a chemical modification of the DNA molecule. Epigenetic factors include the addition or removal of a methyl group which results in changes of the DNA methylation levels.

The term “nucleic acids” as used herein may include any polymer or oligomer of pyrimidine and purine bases, preferably cytosine, thymine, and uracil, and adenine and guanine, respectively. The present invention contemplates any deoxyribonucleotide, ribonucleotide or peptide nucleic acid component, and any chemical variants thereof, such as methylated, hydroxymethylated or glycosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double stranded form, including homoduplex, heteroduplex, and hybrid states.

The term “methylation marker” as used herein refers to a CpG position that is potentially methylated. Methylation typically occurs in a CpG containing nucleic acid. The CpG containing nucleic acid may be present in, e.g., in a CpG island, a CpG doublet, a promoter, an intron, or an exon of gene. For instance, in the genetic regions provided herein the potential methylation sites encompass the promoter/enhancer regions of the indicated genes. Thus, the regions can begin upstream of a gene promoter and extend downstream into the transcribed region.

The term “gene” as used herein refers to a region of genomic DNA associated with a given gene. For example, the region can be defined by a particular gene (such as protein coding sequence exons, intervening introns and associated expression control sequences) and its flanking sequence. It is, however, recognized in the art that methylation in a particular region is generally indicative of the methylation status at proximal genomic sites. Accordingly, determining a methylation status of a gene region can comprise determining a methylation status of a methylation marker within or flanking about 10 bp to 50 bp, about 50 to 100 bp, about 100 by to 200 bp, about 200 by to 300 bp, about 300 to 400 bp, about 400 by to 500 bp, about 500 bp to 600 bp, about 600 to 700 bp, about 700 bp to 800 bp, about 800 to 900 bp, 900 bp to 1 kb, about 1 kb to 2 kb, about 2 kb to 5 kb, or more of a named gene, or CpG position.

The phrase “selectively measuring” as used herein refers to methods wherein only a finite number of methylation marker or genes (comprising methylation markers) are measured rather than assaying essentially all potential methylation marker (or genes) in a genome. For example, in some aspects, “selectively measuring” methylation markers or genes comprising such markers can refer to measuring at least 500, 400, 300, 200, 100, 50 or different methylation markers disclosed herein. In other aspects, “selectively measuring” methylation markers or genes comprising such markers can refer to measuring no more than 500, 400, 300, 200, 100, 50 or 40 different methylation markers disclosed herein.

The invention described herein provides novel and powerful predictors of chronological and epigenetic age, life; expectancy, mortality, and morbidity based on DNA methylation levels. The disclosure presented herein surrounding the prediction of mortality and morbidity show that DNAm based biomarkers are highly robust and informative for a range of applications. DNAm age can not only be used to directly predict/prognosticate age and mortality but also relate to a host of age-related conditions such as heart disease risk, cancer risk, dementia status, cardiovascular disease and various measures of frailty. Further embodiments and aspects of the invention are discussed below.

DNA methylation of the methylation markers can be measured using various approaches, which range from commercial array platforms (e.g. from Illumina™) to sequencing approaches of individual genes. This includes standard lab techniques or array platforms. A variety of methods for detecting methylation status or patterns have been described in, for example U.S. Pat. Nos. 6,214,556, 5,786,146, 6,017,704, 6,265,171, 6,200,756, 6,251,594, 5,912,147, 6,331,393, 6,605,432, and 6,300,071 and US Patent Application Publication Nos. 20030148327, 20030148326, 20030143606, 20030082609 and 20050009059, each of which are incorporated herein by reference. Other array-based methods of methylation analysis are disclosed in U.S. patent application Ser. No. 11/058,566. For a review of some methylation detection methods, see, Oakeley, E. J., Pharmacology & Therapeutics 84:389-400 (1999). Available methods include, but are not limited to: reverse-phase HPLC, thin-layer chromatography, SssI methyltransferases with incorporation of labeled methyl groups, the chloracetaldehyde reaction, differentially sensitive restriction enzymes, hydrazine or permanganate treatment (m5C is cleaved by permanganate treatment but not by hydrazine treatment), sodium bisulfite, combined bisulphate-restriction analysis, and methylation sensitive single nucleotide primer extension.

The methylation levels of a subset of the DNA methylation markers disclosed herein are assayed (e.g. using an Illumina™ DNA methylation array or using a PCR protocol involving relevant primers). To quantify the methylation level, one can follow the standard protocol described by Illumina™ to calculate the beta value of methylation, which equals the fraction of methylated cytosines in that location. The invention can also be applied to any other approach for quantifying DNA methylation at locations near the genes as disclosed herein. DNA methylation can be quantified using many currently available assays which include, for example:
a) Molecular break light assay for DNA adenine methyltransferase activity is an assay that is based on the specificity of the restriction enzyme DpnI for fully methylated (adenine methylation) GATC sites in an oligonucleotide labeled with a fluorophore and quencher. The adenine methyltransferase methylates the oligonucleotide making it a substrate for DpnI. Cutting of the oligonucleotide by DpnI gives rise to a fluorescence increase.
b) Methylation-Specific Polymerase Chain Reaction (PCR) is based on a chemical reaction of sodium bisulfite with DNA that converts unmethylated cytosines of CpG dinucleotides to uracil or UpG, followed by traditional PCR. However, methylated cytosines will not be converted in this process, and thus primers are designed to overlap the CpG site of interest, which allows one to determine methylation status as methylated or unmethylated. The beta value can be calculated as the proportion of methylation.
c) Whole genome bisulfite sequencing, also known as BS-Seq, is a genome-wide analysis of DNA methylation. It is based on the sodium bisulfite conversion of genomic DNA, which is then sequencing on a Next-Generation Sequencing (NOS) platform. The sequences obtained are then re-aligned to the reference genome to determine methylation states of CpG dinucleotides based on mismatches resulting from the conversion of unmethylated cytosines into uracil.
d) The Hpall tiny fragment Enrichment by Ligation-mediated PCR (HELP) assay is based on restriction enzymes' differential ability to recognize and cleave methylated and unmethylated CpG DNA sites.
e) Methyl Sensitive Southern Blotting is similar to the HELP assay but uses Southern blotting techniques to probe gene-specific differences in methylation using restriction digests. This technique is used to evaluate local methylation near the binding site for the probe.
f) ChIP-on-chip assay is based on the ability of commercially prepared antibodies to bind to DNA methylation-associated proteins like MeCP2.
g) Restriction landmark genomic scanning is a complicated and now rarely-used assay is based upon restriction enzymes differential recognition of methylated and unmethylated CpG sites. This assay is similar in concept to the HELP assay.
h) Methylated DNA immunoprecipitation (MeDIP) is analogous to chromatin immunoprecipitation. Immunoprecipitation is used to isolate methylated DNA fragments for input into DNA detection methods such as DNA microarrays (MeDIP-chip) or DNA sequencing (MeDIP-seq).
i) Pyrosequencing of bisulfate treated DNA is a sequencing of an amplicon made by a normal forward primer but a biotinylatod reverse primer to PCR the gene of choice. The Pyrosequencer then analyses the sample by denaturing the DNA and adding one nucleotide at a time to the mix according to a sequence given by the user. If there is a mismatch, it is recorded and the percentage of DNA for which the mismatch is present is noted. This gives the user a percentage methylation per CpG island.

In certain embodiments of the invention, the genomic DNA is hybridized to a complimentary sequence (e.g. a synthetic polynucleotide sequence) that is coupled to a matrix (e.g. one disposed within a microarray). Optionally, the genomic DNA is transformed from its natural state via amplification by a polymerase chain reaction process. For example, prior to or concurrent with hybridization to an array, the sample may be amplified by a variety of mechanisms, some of which may employ PCR. See, for example, PCR Technology: Principles and Applications for DNA Amplification (Ed. H. A. Erlich. Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159, 4,965,188, and 5,333,675. The sample may be amplified on the array. See, for example, U.S. Pat. No. 6,300,070, which is incorporated herein by reference.

In addition to using art accepted modeling techniques on data obtained from embodiments of the invention (e.g. regression analyses), embodiments of the invention can utilize a variety of art accepted technical processes. For example, in certain embodiments of the invention, a bisulfite conversion process is performed so that cytosine residues in the genomic DNA are transformed to uracil, while 5-methylcytosine residues in the genomic DNA are not transformed to uracil. Kits for DNA bisulfite modification are commercially available from, for example. MethylEasy™ (Human Genetic Signatures™) and CpGenome™ Modification Kit (Chemicon™). See also, WO04096825A1, which describes bisulfite modification methods and Olek et al. Nuc. Acids Res. 24:5064-6 (1994), which discloses methods of performing bisulfite treatment and subsequent amplification. Bisulfite treatment allows the methylation status of cytosines to be detected by a variety of methods. For example, any method that may be used to detect a SNP may be used, for examples, see Syvanen, Nature Rev. Gen. 2:930-942 (2001). Methods such as single base extension (SBE) may be used or hybridization of sequence specific probes similar to allele specific hybridization methods. In another aspect the Molecular Inversion Probe (MIP) assay may be used.

The polynucleotides showing genomic sequences having the CpG sites discussed herein are found in Table 1. The Illumina method takes advantage of sequences flanking a CpG locus to generate a unique CpG locus cluster ID with a similar strategy as NCBI's refSNP IDs (rs #) in dbSNP (see, e.g. Technical Note: Epigenetics, CpG Loci Identification ILLUMINA Inc. 2010).

### EXAMPLES

### Example 1: Epigenetic Clocks for Dogs and Humans

The references cited in this Example are found at the end of this Example.

DNA methylation profiles have been used to develop biomarkers of aging known as epigenetic clocks, which predict chronological age with remarkable accuracy and show promise for inferring health status as an indicator of biological age. Epigenetic clocks were first built to monitor human aging but the principles underpinning them appear to be evolutionarily conserved. Here we describe reliable and highly accurate epigenetic clocks shown to apply to 51 domestic dog breeds. The methylation profiles were generated using a custom array with DNA sequences that are conserved across all mammalian species (HorvathMammalMethylChip40). Canine epigenetic clocks were constructed to estimate age. We also present two highly accurate human-dog dual species epigenetic clocks (R=0.97), which may facilitate the ready translation from canine to human use (or vice versa) of anti-aging treatments being developed for longevity and preventive medicine.

### Results

### DNA Methylation Data

All DNA methylation data were generated using the mammalian methylation array (HorvathMammalMethylChip40) that measures cytosine methylation levels in highly conserved regions across mammals (1). We analyzed methylation profiles from 565 blood samples derived from 51 dog breeds (Canis lupus familiaris). The 51 breeds ranged from Bernese mountain dog, with the shortest expected lifespan of eight years (average adult breed weight=41 kg) to Chihuahuas with the highest expected lifespan at 20 years (average adult breed weight=1.8 kg). Expected lifespan estimates were based on the upper limit of lifespan estimated by the American Kennel Club and other registering bodies using sex averaged measures (2). Unsupervised hierarchical clustering demonstrates that methylation profiles grouped by sex above breed, i.e., most females were in one branch while most males grouped in a second branch. We did not find any evidence of grouping by breed.

### Epigenetic Clocks

The dog-only clocks were developed using blood DNA, while the human DNA that was used to generate the human-dog clocks were either from blood or multiple human tissues. The distinction between the two human-dog clocks lies in measurement parameters. One estimates chronological age (in units of years), while the other estimates relative age, which is the ratio of age of an individual to the maximum recorded lifespan of the species, with values between 0 and 1. This ratio allows alignment and biologically meaningful comparison between species with very different lifespans (dog and human), which is not afforded by the simple measurement of chronological age.

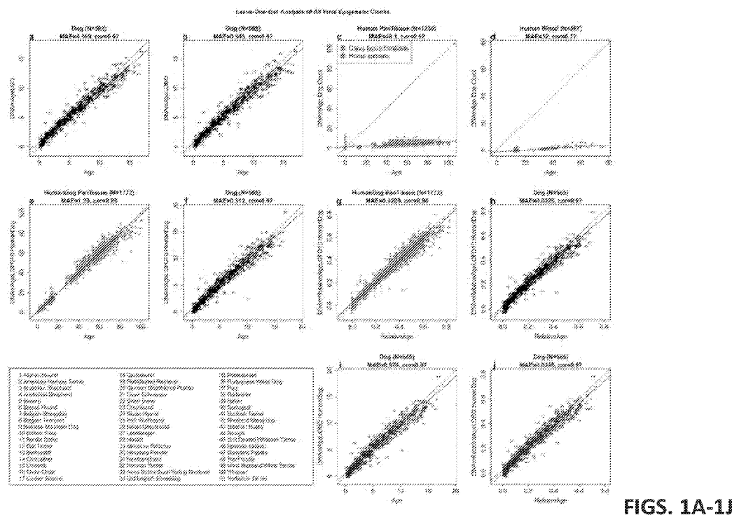

The cross-validation study reports unbiased estimates of the age correlation R, defined as Pearson correlation between the age estimate (DNAm age) and chronological age, as well as the median absolute error. Cross-validation estimates of age correlation for the three dog clocks are 0.97 (FIG. 1a, b, f, h). Different cross validation schemes show that both the pure dog clock and the human-dog clock for chronological age exhibit a median error of less than 0.57 years (seven months) when using blood samples from dogs (FIG. 1a, b, f, i).

By definition, the pure dog clock is not expected to apply to human tissues. However, we observe a remarkably high age correlation in DNA from human blood samples (r=0.77), albeit with a large median error of 32 years (FIG. 1d). The age correlation of the dog clock across a variety of human tissues is lower (r=0.52, FIG. 1c). By contrast, the two human dog clocks are highly accurate in both species (FIG. 1e-j). The human-dog clock for chronological age led to a high age correlation of R=0.99 when both species are analyzed together (FIG. 1e) and remained so when the analysis was restricted to dog samples alone (R=0.97, FIG. 1f). Similarly, the human-dog clock for relative age exhibits a high correlation regardless of whether the analysis is done with samples from both species (R=0.98, FIG. 1g) or only from dogs (R=0.97, FIG. 1h). The impressive accuracy of the human-dog clocks could also be corroborated with an alternative cross validation scheme, i.e., a “leave one dog breed out” (LOBO) cross validation scheme (FIG. 1i,j), which estimates the clock accuracy in dog breeds not used in the training set.

### Epigenetic Clock to Predict Average Time-to-Death

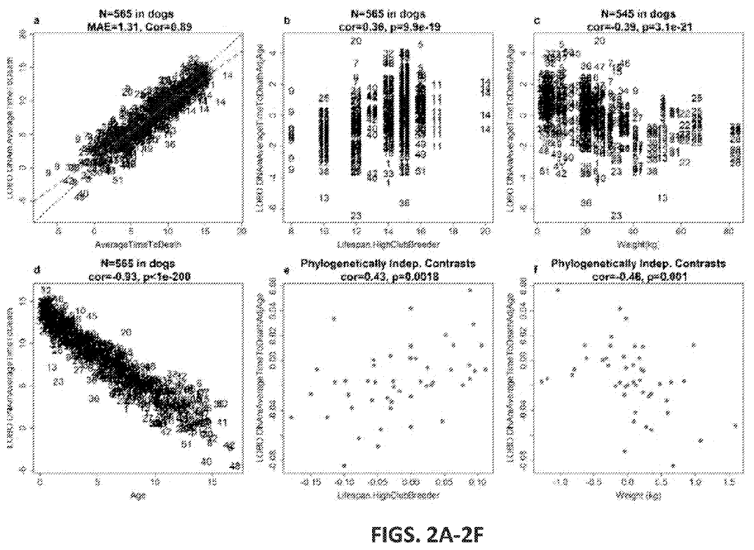

Although epigenetic age clocks can be indirectly employed to predict risk or mortality, their performance may be sub-optimal, as they were developed for the clear purpose of estimating age (3, 4). The DNA methylation data that we generate, however, can be used to develop an epigenetic predictor of average time to death (“DNAmAverageTimeToDeath”), using a penalized regression model (Methods). To evaluate predictive accuracy, we used LOBO cross-validation that divided the dataset into 51 breed groups. At each round, LOBO cross-validation trained each model on all but one breed, which was left out and used for validation at each iteration. LOBO analysis revealed a high correlation (r=0.89. FIG. 2a) between estimated and actual average time to death with a median absolute error (MAE) of 1.3 years. As the successful performance of the LOBO analysis was carried out with 51 distinct breeds, we hypothesize that this model can be extrapolated to breeds not included in our dataset.

We observed that age-adjusted DNAmAverageTimeToDeath was correlated in the expected direction with lifespan (r=0.36 and P=9.9×10-19 FIG. 2b) and average adult breed weight (r=−0.4 and P=3.1×10-21, FIG. 2c). To account for correlated evolution of traits (lifespan, adult weight, etc.) among breeds, we applied phylogenetically independence contrasts (PICs) analysis to our study traits at the breed level. Age-adjusted DNAmAverageTimeToDeath retained similar degree of correlation with lifespan (r=0.43 and P=1.8×10-3, FIG. 2e) and weight (r=−0.46 and P=1.0×10-3. FIG. 2f), respectively. As expected from its construction. DNAmAverageTimcToDeath has a strong negative correlation with chronological age (r=0.93, FIG. 2d). This is consistent with the fact that younger dogs are many more years away from reaching the upper limit of their lifespan for their respective breed. A multivariate regression model shows that the predictive accuracy of DNAmAverageTimeToDeath is retained even after adjusting for chronological age, gender and the average adult weight of the breed (P=1.56×10-3).

### General Background DNA Methylation Based Biomarkers

DNA based biomarker that changes with age is DNA methylation; specifically of cytosine residues of cytosine-phosphate-guanine dinucleotides (CpGs). Machine learning-based analyses of these changes generated algorithms, known as epigenetic clocks that use specific CpG methylation levels to accurately estimate age that is referred to as DNA methylation age (DNAm age)(5-8).

DNAm aging assays are already highly robust and ready for biomarker development; as reported by the BLUEPRINT consortium (9). DNAm based biomarkers are highly promising molecular biomarkers of aging (10, 11).

### Materials and Methods

### Materials

In total, we analyzed DNA from n=565 blood samples (dogs) from 51 breeds. Samples were provided by researchers at the National Human Genome Research Institute (NHGRI) and collection was approved by the Animal Care and Use Committee of the Intramural Program of NHGRI at the National Institutes of Health (Protocol 8329254). The median dog age was 5.6 years (range=0.1-17 years). DNA isolated blood samples from dogs were used to build an epigenetic clock for dogs. To build human-dog clocks we added n=1207 human tissue samples to the training data.

### Lifespan and Breed Characteristics

For each phenotype, we used the average of the standard breed (male+female average). Standard breed weights (SBW), height (SBH) and life span were obtained from several sources: weights and height previously listed in 16, 33, although they were updated if weights specified by the AKC 10 were different. If the AKC did not specify SBW, SBH or life span, we used data from Atlas of Dog Breeds of the World 34. SBW. SBH and life span were applied to all samples from the same breed. Lifespan estimates are available for all dogs within the 51 breeds. Since Bull Terrier and Dachshund breeds have both standard and miniature sizes, the adult weights differ between these two sizes. Therefore, we assigned weight as missing for those breeds in this analysis.

### Human Tissue Samples

To build the human-dog clock, we analyzed previously generated methylation data from n=1207 human tissue samples (adipose, blood, bone marrow, dermis, epidermis, heart, keratinocytes, fibroblasts, kidney, liver, lung, lymph node, muscle, pituitary, skin, spleen) from individuals whose ages ranged from 0 to 93. The tissue samples came from three sources: tissue and organ samples came from the National NeuroAIDS Tissue Consortium 35; blood samples from the Cape Town Adolescent Antiretroviral Cohort study 36; blood, skin and other primary cells were provided by Kenneth Raj 37. All were obtained with Institutional Review Board approval (IRB #15-001454, IRB #16-000471, IRB #18-000315, IRB #16-002028).

### DNA Methylation

All data were generated on the platform (HorvathMammalMethylChip40). The mammalian array provides high coverage (over one thousand-fold) of highly conserved CpGs in mammals, but focuses only on 36k CpGs that are highly conserved across mammals. Out of 37,492 CpGs on the array, 35,988 probes were chosen to assess cytosine DNA methylation levels in mammalian species 9. The particular subset of species for each probe is provided in the chip manifest file which can be found at Gene Expression Omnibus (GEO) at NCBI as platform GPL28271. The SeSaMe normalization method was used to define beta values for each probe 38. Genome coordinates for different dog breeds have been posted on Github as detailed in the section on Data Availability.

### Penalized Regression Models

We developed the six different epigenetic clocks for dogs by regressing chronological age on all CpGs. Age was not transformed. We used all tissues for the pan-tissue clock. We restricted the analysis to blood, liver, and brain tissue for the blood, liver, and brain tissue clocks, respectively. Penalized regression models were created with the R function “glmnet” 39. We investigated models produced by both “elastic net” regression (alpha=0.5). The optimal penalty parameters in all cases were determined automatically by using a 10-fold internal cross-validation (cv.glmnet) on the training set. By definition, the alpha value for the elastic net regression was set to 0.5 (midpoint between Ridge and Lasso type regression) and was not optimized for model performance. We performed a cross-validation scheme for arriving at unbiased (or at least less biased) estimates of the accuracy of the different DNAm based age estimators. One type consisted of leaving out a single sample (LOOCV) from the regression, predicting an age for that sample, and iterating over all samples.

### Relative Age Estimation

To introduce biological meaning into age estimates of dogs and humans with very different lifespans, as well as to overcome the inevitable skewing due to unequal distribution of data points from dogs and humans across age range, relative age estimation is made using the formula: Relative age=Age/maxLifespan where the maximum lifespan for dogs and humans were set to 24 years and 122.5 years, respectively.

Epigenome Wide Association Studies (EWAS) of Age. Lifespans and Weight

EWAS was performed in each tissue separately using the R function “standardScreeningNumericTrait” from the “WGCNA” R package 40.

### Epigenetic Clock for Average Time to Death

We did not have follow-up information (time to death) available for individual dogs in our study. To create a surrogate variable for this important endpoint, we leveraged two other variables: 1) the upper limit of lifespans estimated from the American Kennel Club and other registering bodies (variable name “Lifespan.HighClubBreeder” 10) and 2) the chronological age at the time of the blood draw. For each dog, we defined average time to death (AverageTimeToDeath) as the difference between LifespanHighClubBreeder and chronological age.

To assess the accuracy of the elastic net regression models, we used leave-one-breed-out (LOBO) cross validation. The LOBO cross validation approach trained each model on all but one breeder. The “left out” breed was then used as a test set. The LOBO approach assesses how well the penalized regression models generalize to breeds that were not part of the training data. To ensure unbiased estimates of accuracy, all aspects of the model fitting (including pre-filtering of the CpG) were only conducted in the training data for the LOBO analysis. We fitted the glmnet model to the top 5000 CpGs with the most significant median Z score (lifespan correlation test) in the training data. We average the methylome for each breed and performed EWAS of lifespan (N=50 breeds in training dataset) to select the top 5000 CpGs.

### Practicing the Invention of DNAm Based Biomarkers

To use the epigenetic biomarker one can typically extract DNA from cells or fluids, e.g. blood cells, whole blood, peripheral blood mononuclear cells, saliva, buccal swabs. Next, one needs to measure DNA methylation levels in the underlying signature of CpGs (epigenetic markers) that are being used in the mathematical algorithm. The algorithm leads to an estimate of age for each DNA sample.

### Technical Details Surrounding the DNAm Age Estimator

### Statistical Methods Used for Building the Clocks

The final clocks were used by employing a single elastic net regression model analysis (R function glmnet) on the preliminary training set and final training set, respectively. Details can be found in the scientific publication (Horvath et al 2020). We use used Leave-one-out analysis (LOO) using a single lambda value. We chose the following parameters for the glmnet R function (Alpha: 0.5, CV Fold: 10. Lambda choice for Clock: 1 standard error above minimum CV-MSE).

### Covariates and Coefficient Values of the Dog Clocks

The dog tissue clock is based on 45 CpGs whose coefficient values are specified in the column “Coef.Dog”. Age transformation=identity, i.e. F(Age)=Age

The human dog pan tissue clock for chronological age is based on 505 CpGs whose coefficient values are specified in the column “Coef.HumanDogPanTissueLogLinearAge”. Age transformation=log-linear described below. The human dog blood clock for chronological age is based on 109 CpGs whose coefficient values are specified in the column “Coef.HumanDogBlood”. Age transformation=identity.

The human dog pan tissue clock for relative age is based on 473 CpGs whose coefficient values are specified in the column “Coef.HumanDogPanTissueRelativeAge”. Age transformation: relative age. i.e. F(Age)=Age/maxLifespan where the maximum lifespan for dogs and humans were set to 24 years and 122.5 years, respectively.

The human dog blood clock for relative age is based on 110 CpGs whose coefficient values are specified in the column “Coef.HumanDogBloodRelativeAge”. Age transformation: relative age. i.e. F(Age)=Age/maxLifespan.

Epigenetic estimator of average time to death is based on 367 CpGs whose coefficient values are specified in the column “Coef.AverageTimeToDeath”. Age transformation=identity, i.e. F(Age)=Age

General description of age transformation The human-dog clocks for chronological age used log linear transformations that are similar to those employed for the HUMAN pan tissue (Horvath 2013) (12).

Thus, the dependent variable, chronological age, was transformed before carrying out an elastic net regression analysis. Toward this end, the function F(x) where the argument is an age estimate.

Note that F satisfies the following desirable properties: it
i) is a continuous, monotonically increasing function (which can be inverted),
ii) has a logarithmic dependence during development
iii) has a linear dependence on age after development
iv) is defined for negative ages (i.e. prenatal samples)
v) it has a continuous first derivative (slope function).

An elastic net regression model (implemented in the glmnet R function) was used to regress a transformed version of age on the beta values in the training data. The glmnet function requires the user to specify two parameters (alpha and beta). Since I used an elastic net predictor, alpha was set to 0.5. But the lambda value of was chosen by applying a 10 fold cross validation to the training data (via the R function cv.glmnet).

The elastic net regression results in a linear regression model whose coefficients b0, b1, . . . , relate to transformed age as follows

$$F ( chronological age ) = b 0 + b 1 CpG 1 + … + bpCpGp + error$$

Note that the intercept term is denoted by b0.

Based, on the coefficient values from the regression model, DNAmAge is estimated as follows

$$DNAmAge = {F}^{⋀} ( - 1 ) ( b 0 + b 1 CpG 1 + … + bpCpGp )$$

where F\hat{ }(−1) (y) denotes the mathematical inverse of the function F(·) Thus, the regression model can be used to predict to transformed age value by simply plugging the beta values of the selected CpGs into the formula.

### Defining Properties of the Log Linear Transformation

As indicated by its name, the “log-linear” function, has a logarithmic dependence on age before the average age of sexual maturity (of the species) and a linear dependence after Age at Sexual Maturity (of the species). For the human-dog clocks we used the following averages at sexual maturity (in units of years): 13.5 years for humans and 1.40 years for dogs.

### Construction

We used a piecewise transformation, parameterized by Age of Sexual Maturity (A).

The transformation is F(x), given by

$$F ( x ) = g ( \frac{x + 1.5}{A + 1.5} ) where p ( t ) = \{ \begin{matrix}log ( t ) , & for 0 \leq t \leq 1 \\ t - 1 , & for 1 \leq t\end{matrix}$$

Explicitly. F(x) is given by

$$F ( x ) = \{ \begin{matrix}log ( \frac{x + 1.5}{A + 1.5} ) , & for 0 \leq x \leq A \\ \frac{x - A}{A + 1.5} , & for A \leq x\end{matrix}$$

In order to use this transformation to predict Age on new samples, one needs to use the inverse transformation, F⁻¹(y), given by

$${F}^{- 1} ( y ) = \{ \begin{matrix}( A + 1.5 ) * exp ( y ) - 1.5 , & for y \leq 0 \\ ( A + 1.5 ) y + A , & for y \geq 0\end{matrix}$$

For predicting age, you will apply the inverse transformation to coefficient-weighted sum.

That is,

$$DNAmAge = {F}^{- 1} ( x * β )$$

where β is the vector of coefficients and x is the vector of methylation values, with an intercept term.

The DNAm Age estimate is estimated in two steps.

First, one forms a weighted linear combination of the CpGs whose details can be found, for example, in Tables 100-107 of U.S. Provisional Patent Application Ser. No. 63/215,289, the contents of which are incorporated by reference.

The table reports the probe identifier (cg number) used in the custom Infinium array (HorvathMammalMethylChip40). The weights used in this linear combination are specified in the respective column entitled “Coef”.

The formula assumes that the DNA methylation data measure “beta” values but the formula could be adapted to other ways of generating DNA methylation data.

Second, the weighted average of the CpGs is transformed using a monotonically increasing function so that it is in units of years.

$$DNAmAge = {F}^{⋀} ( - 1 ) ( WeightedAverage )$$

A novel aspect of the above-noted invention is the development of epigenetic biomarkers that apply to two species (dogs and humans) at the same time. A single mathematical formulas based on the same methylation probes can be used to measure age in both species based on any tissue sample (i.e. these are pan tissue clocks). The fact these epigenetic biomarkers apply to both species greatly increases the likelihood that findings from preclinical studies in dogs will actually translate to humans.

One of the human-dog clocks measures relative age (defined as ratio of age by maximum lifespan). This clock puts both species on the same footing. The relative age of 0.5 corresponds to 61 years in humans (half of 122 years) and 12 years in dogs (half of 24 years).

Novel “biomarkers of aging”, i.e. assessments that allow one to measure age, are interesting to gerontologists (aging researchers), anti-aging researchers, pharmaceutical companies that carry out preclinical studies.

Overall, we expect that these epigenetic biomarkers will become useful biomarkers for preclinical studies using dogs because they capture the physiological aging state, thus allowing efficacy of interventions to be evaluated based on real-time measures of aging, rather than relying on long-term outcomes, such as morbidity and mortality.

Finally, this measure may be another component of other molecular biomarkers of aging.

In summary, the invention provides novel epigenetic biomarker of aging. Strikingly, some of these biomarkers apply to two species: dogs and humans.

It is critical to distinguish molecular biomarkers such as DNAm Age from clinical biomarkers of aging. Clinical biomarkers such as lipid levels, blood pressure, blood cell counts have a long and successful history in clinical practice. By contrast, molecular biomarkers of aging are rarely used. However, this is likely to change due to recent breakthroughs in DNA methylation based biomarkers of aging. DNA methylation (DNAm) based biomarkers of aging promise to greatly enhance biomedical research, clinical applications, and preclinical studies. They will also be more useful for preclinical studies and intervention assessment that target aging, since they are more proximal to the biological changes that characterize the aging process compared to upstream clinical read outs of health and disease status.

While these DNAm based biomarkers will probably not replace traditional biomarker assessments, they provide complementary information that adds valuable information, with preclinical applications.

### REFERENCES

1. Arneson A. Haghani A, Thompson M J, Pellegrini M. Kwon S B, Vu H, Li C Z, Lu A T, Barnes B. Hansen K D, et al: A mammalian methylation array for profiling methylation levels at conserved sequences. bioRxiv 2021:2021.2001.2007.425637.
2. TheAmericanKennelClub: The Complete Dog Book: 20th Edition. 20th Edition edn. New York, NY: Howell Book House, 2006.
3. Horvath S, Raj K: DNA methylation-based biomarkers and the epigenetic clock theory of ageing. Nat Rev Genet 2018.
4. Lu A T, Quach A, Wilson J G, Reiner A P, Aviv A, Raj K, Hou L, Baccarelli A A, Li Y, Stewart J D, et al: DNA methylation GrimAge strongly predicts lifespan and healthspan. Aging (Albany NY) 2019, 11:303-327.
5. Hannum G: Genome-wide methylation profiles reveal quantitative views of human aging rates. Mol Cell 2013, 49.
6. Lin Q, Weidner C I, Costa I G, Marioni R E, Ferreira M R P, Deary I J: DNA methylation levels at individual age-associated CpG sites can be indicative for life expectancy. Aging 2016, 8:394-401.
7. Horvath S: DNA methylation age of human tissues and cell types. Genome Biol 2013, 14.
8. Horvath S, Oshima J, Martin G M, Lu A T, Quach A, Cohen H, Felton S, Matsuyama M, Lowe D, Kabacik S, et al: Epigenetic clock for skin and blood cells applied to Hutchinson Gilford Progeria Syndrome and ex vivo studies. Aging (Albany NY) 2018, 10:1758-1775.
9. The Bc, Bock C, Halbritter F, Carmona F J, Tierling S, Datlinger P. Assenov Y, Berdasco M, Bergmann A K, Booher K, et al: Quantitative comparison of DNA methylation assays for biomarker development and clinical applications. Nature Biotechnology 2016, 34:726.
10. Jylhava J. Pedersen N L, Hagg S: Biological Age Predictors. EBioMedicine 2017, 21:29-36.
11. Horvath S, Raj K: DNA methylation-based biomarkers and the epigenetic clock theory of ageing. Nat Rev Genet 2018, 19:371-384.
12. Horvath S: DNA methylation age of human tissues and cell types. Genome Biol 2013, 14:R115.
13. Bocklandt S, Lin W. Sehl M E, Sanchez F J, Sinsheimer J S, Horvath S, Vilain E: Epigenetic predictor of age. PLoS One 2011, 6.
14. Weidner C I: Aging of blood can be tracked by DNA methylation changes at just three CpG sites. Genome Biol 2014, 15.
15. Hannum G, Guinney J, Zhao L. Zhang L, Hughes G, Sadda S, Klotzle B, Bibikova N, Fan J B, Gao Y, et al: Genome-wide methylation profiles reveal quantitative views of human aging rates. Mol Cell 2013. 49:359-367.
16. Marioni R, Shah S, McRae A, Chen B, Colicino E, Harris S, Gibson J, Henders A, Redmond P, Cox S, et al: DNA methylation age of blood predicts all-cause mortality in later life. Genome Biol 2015, 16:25.
17. Christiansen L, Lenart A, Tan Q, Vaupel J W, Aviv A, McGue M, Christensen K: DNA methylation age is associated with mortality in a longitudinal Danish twin study. Aging Cell 2015.
18. Pema L, Zhang Y, Mons U. Holleczek B. Saum K U, Brenner H: Epigenetic age acceleration predicts cancer, cardiovascular, and all-cause mortality in a German case cohort. Clinical Epigenetics 2016, 8:1-7.

### Example 2: Epigenetic Clocks for Rats and Humans

The references cited in this Example are found at the end of this Example.

When considering the concept of aging and rejuvenation, it is important to appreciate that improved health or organ function through medication or surgery does not necessarily indicate molecular age reversal. Hence, it is conceptually challenging to test whether plasma fraction treatment, or any other putative treatment, actually reverses biological age, because there is no consensus on how to measure biological aging (1). We addressed this challenge by using both clinical biomarkers and molecular biomarkers of aging. While clinical biomarkers have obvious advantages (being indicative of organ dysfunction or disease), they are neither sufficiently mechanistic nor proximal to fundamental mechanisms of aging to serve as indicators of them. It has long been recognized that epigenetic changes are one of several primary hallmarks of aging (2-6). With the technical advancement of methylation army platforms that can provide quantitative and accurate profiles of specific CpG methylations: came the insight to combine methylation levels of several DNA loci to develop an accurate age estimator (7-11). Such DNA methylation (DNAm) age estimators exhibit unexpected properties: they apply to all sources of DNA (sorted cells, tissues, and organs) and surprisingly to the entire age spectrum (from prenatal tissue samples to tissues of centenarians) (10, 12). A substantial body of literature demonstrates that these epigenetic clocks capture aspects of biological age (12). This is demonstrated by the finding that the discrepancy between DNAm age and chronological age (term as “epigenetic age acceleration”) is predictive of all-cause mortality even after adjusting for a variety of known risk factors (13-15). Pathologies and conditions that are associated with epigenetic age acceleration includes, but are not limited to, cognitive and physical functioning (16), centenarian status (15, 17), Down syndrome (18), HIV infection (19), obesity (20) and early menopause (21).

We demonstrated that the human pan-tissue clock can be directly applied to chimpanzee DNA methylation profiles (10), but its performance with profiles of other animals decline as a result of evolutionary genome sequence divergence. Recently, epigenetic clocks for mice were developed and used successfully to evaluate and confirm gold-standard longevity interventions such as calorie restriction and ablation of growth hormone receptor (22-27). These observations strongly suggest that age-related DNA methylation change is an evolutionarily conserved trait and as such, accurate age estimators as those developed for humans can be applied across species. Here we describe the development and performance of different epigenetic clocks for rats. Some of these epigenetic clocks apply to both humans and rats (dual species clocks). We used these epigenetic clocks to evaluate the plasma fraction-based treatment in 4 rat tissues from 2-year-old rats.

### Results

### DNA Methylation Data

All DNA methylation data were generated on a custom methylation array that applies to all mammals. We obtained in total, DNA methylation profiles of over n=553 samples from 13 different tissues of rat (Rattus norvegicus), with ages that ranged from 0.0384 years (i.e. 2 weeks) to 2.3 years (i.e. 120 weeks). The rat tissue samples came from 3 different countries: (i) India (Nugenics Research in collaboration with NMIMS School of Pharmacy's), (ii) the United States (H. Chen and L. Solberg Woods), and (iii) Argentina (R. Goya). Unsupervised hierarchical clustering shows that the methylation profiles clustered by tissue type, as would be expected.

Our DNA methylation-based age estimators (epigenetic clocks) were developed (“trained” in the parlance of machine learning) using the rat tissues. The two epigenetic clocks that apply to both species were developed by adding n=1207 human tissue samples to the rat training set. Both rat and human tissues were profiled on the same methylation array platform (HorvathMammalMethylChip40) that focuses on 36,000 highly conserved CpGs (Methods).

### Epigenetic Clocks

Our six different clocks for rats can be distinguished along several dimensions (tissue type, species, and measure of age). Some clocks apply to all tissues (pan-tissue clocks) while others are tailor-made for specific tissues/organs (brain, blood, liver). The rat pan-tissue clock was trained on all available tissues. The brain clock was trained using DNA samples extracted from whole brain, hippocampus, hypothalamus, neocortex, substantia nigra, cerebellum, and the pituitary gland. The liver and blood clock were trained using the liver and blood samples from the training set, respectively. While the four rat clocks (pan-tissue-, brain-, blood-, and liver clocks) apply only to rats, the human-rat clocks apply to both species. The two human-rat pan-tissue clocks are distinct, by way of measurement parameters. One estimates chronological age (in units of years), while the other estimates relative age, which is the ratio of chronological age to maximum lifespan; with values between 0 and 1. This ratio allows alignment and biologically meaningful comparison between species with very different lifespan (rat and human), which is not afforded by mere measurement of chronological age.

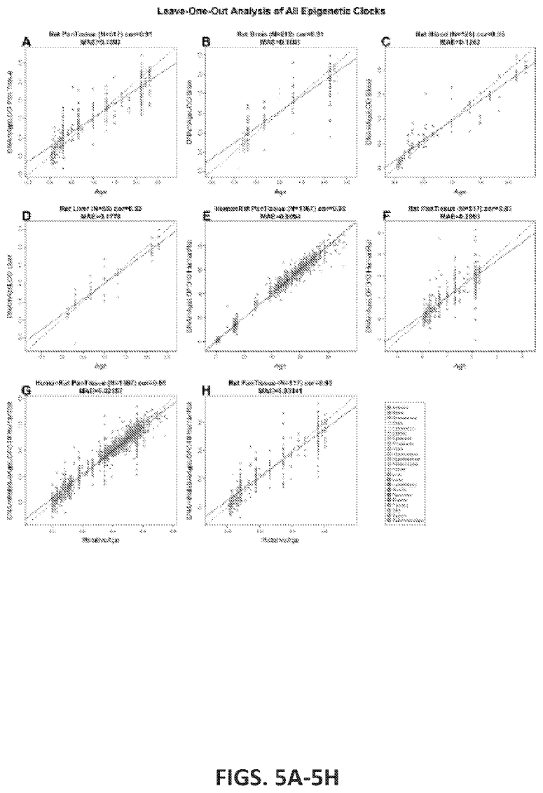

To arrive at unbiased estimates of the six epigenetic clocks, we used a) cross-validation of the training data and b) evaluation with an independent test data set. The cross-validation study reports unbiased estimates of the age correlation R (defined as Pearson correlation between the age estimate (DNAm age) and chronological age) as well as the median absolute error (FIG. 5). The cross-validation estimates of the age correlations for all six clocks are higher than 0.9. The four rat clocks exhibited median absolute errors that range from 0.12 years (1.4 months) for the rat blood clock to 0.189 years (2.3 months) for the rat pan-tissue clock, FIG. 5A-D). The human-rat clock for age generated an age correlation of R=0.99 when both species are analyzed together (FIG. 5E) but is lower when the analysis is restricted to rat tissues alone (R=0.83, FIG. 5F). In contrast, the human-rat clock for relative age exhibits high correlation regardless of whether the analysis is done with samples from both species (R=0.96, FIG. 5G) or only with rat samples (R=0.92, FIG. 5H). This demonstrates that relative age circumvents the skewing that is inherent when chronological age of species with very different lifespans are measured using a single formula. This is due in part to the unequal distribution of training data at the opposite ends of the age range.

As indicated by its name, the rat pan-tissue clock is highly accurate in age estimation of all the different tissue samples tested. We also evaluated the accuracy of the six epigenetic clocks in independent test data from the plasma fraction test study. In the untreated rat tissue samples, the epigenetic clocks exhibited high age correlations in all tissues (R>=0.95 in blood, liver, and the hypothalamus and R>=0.86 in Heart Tissue).

### Development of Rat Epigenetic Clocks

Epigenetic clocks for humans have found many biomedical applications including the measure of age in human clinical trials (12, 28). These clocks provide a standard measure of DNA methylation state in function of chronological age. As impressive as its accuracy is, it is the divergence from this standard that was particularly important because it uncovered the association between accelerated epigenetic age and the associated increased risk of a host of conditions and pathologies, indicating that epigenetic clocks are associated with biological age. This instigated development of similar clocks for animals, of which the ones for mice were particularly attractive as they allow for epigenetic age to be modeled in a mouse system, and at the same time allows existing mouse models of aging to be interrogated with regards to epigenetic aging. Indeed, numerous mouse epigenetic clocks have since been developed and successfully validated against factors, such as rapamycin, caloric restriction and growth factor ablation, which are all well-characterized in their effects on aging of mice (22-27). While the advantages of mouse as a biological model lies in no small part to their size, this also poses a limitation in studies that require regular interval collection of sufficient amounts of blood for analyses, as was the case in the second part of this study. The development of six rat epigenetic clocks described here was based on novel DNA methylation data that were derived from thirteen rat tissue types. The two human-rat clocks demonstrate the feasibility of building epigenetic clocks for two species based on a single mathematical formula. A critical step toward crossing the species barrier was the use of a mammalian DNA methylation array that profiled 36 thousand probes that were highly conserved across numerous mammalian species. The rat DNA methylation profiles represent the most comprehensive dataset thus far of matched single base resolution methylomes in rats across multiple tissues and ages. We expect that the availability of these clocks and their impressive performance in the second part of this study will provide a significant boost to the attractiveness of the rat as biological model in aging research.

Beyond their utility, these clocks reveal several salient features with regards to the biology of aging. First, the rat pan-tissue clock re-affirms the implication of the human pan-tissue clock, which is that aging might be a coordinated biological process that is harmonized throughout the body. Given that the circulatory system irrigates and connects all the organs, it is more likely than not, that the regulation and harmonization of age are mediated systemically. Second, the ability to combine these two pan-tissue clocks into a single human-rat pan-tissue clock attests to the high conservation of the aging process across two evolutionary distant species. This implies, albeit does not guarantee, that treatments that alter the epigenetic age of rats, as measured using the human-rat clock is likely to exert similar effects in humans. If validated, this would be a step change in aging research. Although conservation of aging mechanism could be equally deduced from the existence of multiple individual clocks for other mammals (mouse, dog), the single formula of the human-rat clock that is equally applicable to both species effectively demonstrates this fact. It is evident that the mechanism underpinning aging is a very primitive and important biological process, which ensured its conservation across the mammalian kingdom through time.

The incorporation of two species with very different lifespans such as rat and human, raises the inevitable challenge of unequal distribution of data points along the age range. The clustering of the shorter and longer lifespan species at the lower and higher age range respectively can skew the accuracy of the clock when it is applied individually to either of the species. This effect is mitigated by the generation of the human-rat pan-tissue relative age clock which embeds the estimated age in context of the maximal lifespan recorded for the of the relevant species. In addition to minimizing the skew, this mathematical operation also generates a much more biologically meaningful value because it indicates the relative biological age and fitness of the organism in relation to its own species. This principle will be an important feature to incorporate in future composite epigenetic clocks of different species.

### General Background DNA Methylation Based Biomarkers

DNA based biomarker that changes with age is DNA methylation; specifically of cytosine residues of cytosine-phosphate-guanine dinucleotides (CpGs). Machine learning-based analyses of these changes generated algorithms, known as epigenetic clocks that use specific CpG methylation levels to accurately estimate age that is referred to as DNA methylation age (DNAm age)(29-32).

DNAm aging assays are already highly robust and ready for biomarker development; as reported by the BLUEPRINT consortium (33). DNAm based biomarkers are highly promising molecular biomarkers of aging (34, 35).

### Materials and Methods

In total, we analyzed n=593 rat tissue samples from 13 different sources of DNA. Ages ranged from 0.0384 years (i.e. 2 weeks) to 2.3 years (i.e. 120 weeks). The rat data were comprised of a training set (n=503) and a test set (n=76). To build human-rat clocks, we added n=1207 human tissue samples to the training data. We first trained/developed epigenetic clocks using the training data (n=503 tissues). Next, we evaluated the data in independent test data (n=76 for evaluating the effect of plasma fraction treatment. We used n=503 tissue to train 4 clocks: a pan-tissue clock based on all available tissues, a brain clock based on regions of the whole brain—hippocampus, hypothalamus, neocortex, substantia nigra, cerebellum, and the pituitary gland, a liver clock based on all liver samples, and a blood clock.

Tissue sample collection: Before sacrifice by decapitation, rats were weighed, blood was withdrawn from the tail veins with the animals under isoflurane anesthesia and collected in tubes containing 10 μl EDTA 0.342 mol/l for 500 μl blood. The brain was removed carefully severing the optic and trigeminal nerves and the pituitary stalk (not to tear the pituitary gland), weighed and placed on a cold plate. All brain regions were dissected by a single experimenter (see below). The skull was handed over to a second experimenter in charge of dissecting and weighing the adenohypophysis. The rest of the body was handed to other 2 or 3 experimenters who dissected and collected whole ovaries, a sample of liver tissue, adipose tissue and skin tissue from the distal portion of tails.

Brain region dissection: Prefrontal cortex, hippocampus, hypothalamus, substantia nigra and cerebellum were rapidly dissected on a cold platform to avoid tissue degradation. After dissection, each tissue sample was immediately placed in a 1.5 ml tube and momentarily immersed in liquid nitrogen. The brain dissection protocol was as follows. First a frontal coronal cut was made to discard the olfactory bulb, then the cerebellum was detached from the brain and from the medulla oblongata using forceps. To isolate the medial basal hypothalamus (MBH), brains were placed ventral side up and a second coronal cut was made at the center of the median eminence (−3.6 mm referred to bregma). Part of the MBH was taken from the anterior block of the brain and the other part from the posterior block in both cases employing forceps. The hippocampus was dissected from cortex in both hemispheres using forceps. This procedure was also performed on the anterior and posterior blocks, alternatively placing the brain caudal side up and rostral side up. To dissect the substantia nigra, in each hemisphere a 1-mm thick section of tissue was removed from the posterior part of the brain (−4.6 mm referred to bregma) using forceps. Finally, the anterior block was placed dorsal side up, to separate prefrontal cortex. With a sharp scalpel, a cut was made 2 mm from the longitudinal fissure, and another cut was made 5 mm from it. Additionally, two perpendicular cuts were made, 3 mm and 6 mm from the most rostral point, obtaining a 9 mm2 block of prefrontal cortex. This procedure was performed in both hemispheres and the two prefrontal regions collected in a code-labeled tube.

### Human Tissue Samples

To build the human-rat clock, we analyzed previously generated methylation data from n=1207 human tissue samples (adipose, blood, bone marrow, dermis, epidermis, heart, keratinocytes, fibroblasts, kidney, liver, lung, lymph node, muscle, pituitary, skin, spleen) from individuals whose ages ranged from 0 to 93. The tissue samples came from three sources. Tissue and organ samples from the National NeuroAIDS Tissue Consortium (36). Blood samples from the Cape Town Adolescent Antiretroviral Cohort study (37). Skin and other primary cells provided by Kenneth Raj (38). Ethics approval (IRB #15-001454, IRB #16-000471. IRB #18-000315, IRB #16-002028).

### Practicing the Invention of DNAm Based Biomarkers

To use the epigenetic biomarker one can typically extract DNA from cells or fluids, e.g. blood cells, whole blood, peripheral blood mononuclear cells, liver tissue, skin. Next, one needs to measure DNA methylation levels in the underlying signature of CpGs (epigenetic markers) that are being used in the mathematical algorithm. The algorithm leads to an estimate of age for each DNA sample.

### Technical Details Surrounding the DNAm Age Estimator

We developed twelve epigenetic clocks for rats: six “preliminary” version (based on n=503 rat tissues from the training data) and six “final” versions (based on n=553 samples resulting from combining the 503 rat tissues with n=50 tissue samples from untreated rats in the test data).

The different clocks for rats can be distinguished along several dimensions (tissue type, species, and measure of age). Some clocks apply to all tissues (pan-tissue clocks) while others are tailor-made for specific tissues/organs (brain, blood, liver). The rat pan-tissue clock was trained on all available tissues. The brain clock was trained using DNA samples extracted from whole brain, hippocampus, hypothalamus, neocortex, substantia nigra, cerebellum, and the pituitary gland. The liver and blood clock were trained using the liver and blood samples from the training set, respectively. While the four rat clocks (pan-tissue-, brain-, blood-, and liver clocks) apply only to rats, the human-rat clocks apply to both species. The two human-rat pan-tissue clocks are distinct, by way of measurement parameters. One estimates chronological age (in units of years), while the other estimates relative age, which is the ratio of chronological age to maximum lifespan; with values between 0 and 1. This ratio allows alignment and biologically meaningful comparison between species.

### Penalized Regression Models

We developed the six different epigenetic clocks for rats by regressing chronological age on all CpGs that are known to map to the genome or Rattus norvegicus. Age was not transformed. We used all tissues for the pan-tissue clock. We restricted the analysis to blood, liver, and brain tissue for the blood, liver, and brain tissue clocks, respectively. Penalized regression models were created with the R function “glmnet” (39). We investigated models produced by both “elastic net” regression (alpha=0.5). The optimal penalty parameters in all cases were determined automatically by using a 10 fold internal cross-validation (cv.glmnet) on the training set. By definition, the alpha value for the elastic net regression was set to 0.5 (midpoint between Ridge and Lasso type regression) and was not optimized for model performance. We performed a cross-validation scheme for arriving at unbiased (or at least less biased) estimates of the accuracy of the different DNAm based age estimators. One type consisted of leaving out a single sample (LOOCV) from the regression, predicting an age for that sample, and iterating over all samples.

### Relative Age Estimation

To introduce biological meaning into age estimates of rats and humans that have very different lifespan; as well as to overcome the inevitable skewing due to unequal distribution of data points from rats and humans across age range, relative age estimation was made using the formula: Relative age=Age/maxLifespan where the maximum lifespan for rats and humans were set to 3.8 years and 122.5 years, respectively.

### Final Version of Epigenetic Clocks

The final versions of our epigenetic clocks are meant for future studies of rat tissue samples. These final versions of clocks were developed by combining the original training data (n=503 rat tissues) with the “untreated” samples from the rat test data. Increasing the sample size of the training data leads to a higher accuracy according to a cross validation analysis. Using the final version of the epigenetic clocks, we find that the treatment effects become even more significant especially for the hypothalamus.

### Statistical Methods Used for Building the Clocks

The final clocks were used by employing a single elastic net regression model analysis (R function glmnet) on the preliminary training set and final training set, respectively. Details can be found in the scientific publication (Horvath et al 2020). We use used Leave-one-out analysis (LOO) using a single lambda value. We chose the following parameters for the glmnet R function (Alpha: 0.5, CV Fold: 10, Lambda choice for Clock: 1 standard error above minimum CV-MSE).

### Covariates and Coefficient Values of the Rat Clocks

The final rat pan tissue clock is based on 196 CpGs whose coefficient values are specified in the column “Coef.RatPanTissue”. Age transformation=identity, i.e. F(Age)=Age
The final rat blood clock is based on 51 CpGs whose coefficient values are specified in the column “Coef.RatBlood”. Age transformation=identity, i.e. F(Age)=Age
The final rat liver clock is based on 46 CpGs whose coefficient values are specified in the column “Coef.RatLiver”. Age transformation=identity, i.e. F(Age)=Age
The final rat brain clock is based on 108 whose coefficient values are specified in the column “Coef.RatBrain”. Age transformation=identity, i.e. F(Age)=Age
The final human rat clock for chronological age is based on 701 CpGs whose coefficient values are specified in the column “Coef.HumanRatLogLinearAge”. Age transformation=identity, i.e. F(Age)=LogLinear(Age)
The final human rat clock for relative age is based on 621 CpGs whose coefficient values are specified in the column “Coef.HumanRatRelativeAge”. Age transformation: relative age. i.e. F(Age)=Age/maxLifespan where the maximum lifespan for rats and humans were set to 3.8 years and 122.5 years, respectively.

General description of age transformation The human-rat clocks for chronological age used log linear transformations that are similar to those employed for the HUMAN pan tissue (Horvath 2013) (10).

Thus, the dependent variable, chronological age, was transformed before carrying out an elastic net regression analysis. Toward this end, the function F(x) where the argument is an age estimate.

Note that F Satisfies the Following Desirable Properties: It
i) is a continuous, monotonically increasing function (which can be inverted),
ii) has a logarithmic dependence during development
iii) has a linear dependence on age after development
iv) is defined for negative ages (i.e. prenatal samples)
v) it has a continuous first derivative (slope function).

An elastic net regression model (implemented in the glmnet R function) was used to regress a transformed version of age on the beta values in the training data. The glmnet function requires the user to specify two parameters (alpha and beta). Since I used an elastic net predictor, alpha was set to 0.5. But the lambda value of was chosen by applying a 10 fold cross validation to the training data (via the R function cv glmnet).

The elastic net regression results in a linear regression model whose coefficients b0, b1, . . . , relate to transformed age as follows

$$F ( chronological age ) = b 0 + b 1 CpG 1 + … + bpCpGp + error$$

Note that the intercept term is denoted by b0.

Based, on the coefficient values from the regression model, DNAmAge is estimated as follows

$$DNAmAge = {F}^{⋀} ( - 1 ) ( b 0 + b 1 CpG 1 + … + bpCpGp )$$

where F\hat{ }(−1) (y) denotes the mathematical inverse of the function F(·). Thus, the regression model can be used to predict to transformed age value by simply plugging the beta values of the selected CpGs into the formula.

### Defining Properties of the Log Linear Transformation

As indicated by its name, the “log-linear” function, has a logarithmic dependence on age before the average age of sexual maturity (of the species) and a linear dependence after Age at Sexual Maturity (of the species). For the human-rat clocks we used the following averages at sexual maturity (in units of years): 13.5 years for humans and 0.219178082191781 years for rats.

### Construction

We used a piecewise transformation, parameterized by Age of Sexual Maturity (A).

The transformation is F(x), given by

$$F ( x ) = g ( \frac{x + 1.5}{A + 1.5} ) where g ( t ) = \{ \begin{matrix}log ( t ) , & for 0 \leq t \leq 1 \\ t - 1 , & for 1 \leq t\end{matrix}$$

Explicitly, F(x) is given by

$$F ( x ) = \{ \begin{matrix}log ( \frac{x + 1.5}{A + 1.5} ) , & for 0 \leq x \leq A \\ \frac{x - A}{A + 1.5} , & for A \leq x\end{matrix}$$

In order to use this transformation to predict Age on new samples, one needs to use the inverse transformation, F⁻¹(y), given by

$$F \text{?} ( y ) = \{ \begin{matrix}( A + 1.5 ) \text{?} exp ( y ) - 1.5 , for y < 0 \\ ( A + 1.5 ) y + A , for y \geq 0\end{matrix} \\ \text{?} \text{indicates text missing or illegible when filed}$$

For predicting age, you will apply the inverse transformation to coefficient-weighted sum.

That is,

$$DNAmAge = {F}^{-} \text{?} ( x * β ) \\ \text{?} \text{indicates text missing or illegible when filed} \\$$

where β is the vector of coefficients and x is the vector of methylation values, with an intercept term.

### The DNAm Age Estimate is Estimated in Two Steps.

First, one forms a weighted linear combination of the CpGs.

The weights used in this linear combination can be specified in the respective column entitled “Coef.”.

The formula assumes that the DNA methylation data measure “beta” values but the formula could be adapted to other ways of generating DNA methylation data.

Second, the weighted average of the CpGs is transformed using a monotonically increasing function so that it is in units of years.

$$DNAmAge = F \hat{} ( - 1 ) ( WeightedAverage )$$

A novel aspect of the above-noted invention is the development of epigenetic biomarkers that apply to two species (rats and humans) at the same time. A single mathematical formulas based on the same methylation probes can be used to measure age in both species based on any tissue sample (i.e. these are pan tissue clocks). The fact these epigenetic biomarkers apply to both species greatly increases the likelihood that findings from preclinical studies in rats will actually translate to humans.

One of the human-rat clocks measures relative age (defined as ratio of age by maximum lifespan). This clock puts both species on the same footing. The relative age of 0.5 corresponds to 61 years in humans (half of 122 years) and 1.9 years in rats (half of 3.8 years).

While there are several published clocks for mice and humans, these are the first epigenetic clocks for rats that I am aware of.

Novel “biomarkers of aging”, i.e assessments that allow one to measure age, are interesting to gerontologists (aging researchers), anti-aging researchers, pharmaceutical companies that carry out preclinical studies.

Overall, we expect that these epigenetic biomarkers will become useful biomarkers for preclinical studies using rats because they capture the physiological aging state, thus allowing efficacy of interventions to be evaluated based on real-time measures of aging, rather than relying on long-term outcomes, such as morbidity and mortality.

Finally, this measure may be another component of other molecular biomarkers of aging.

### Competitive Advantage

In summary, the invention provides novel epigenetic biomarker of aging. Strikingly, some of these biomarkers apply to two species: rats and humans.

It is critical to distinguish molecular biomarkers such as DNAm Age from clinical biomarkers of aging. Clinical biomarkers such as lipid levels, blood pressure, blood cell counts have a long and successful history in clinical practice. By contrast, molecular biomarkers of aging are rarely used. However, this is likely to change due to recent breakthroughs in DNA methylation based biomarkers of aging. DNA methylation (DNAm) based biomarkers of aging promise to greatly enhance biomedical research, clinical applications, and preclinical studies. They will also be more useful for preclinical studies and intervention assessment that target aging, since they are more proximal to the biological changes that characterize the aging process compared to upstream clinical read outs of health and disease status.

While these DNAm based biomarkers will probably not replace traditional biomarker assessments, they provide complementary information that adds valuable information, with preclinical applications.

### REFERENCES

1. Ferrucci L, Gonzalez-Freire M, Fabbri E, Simonsick E. Tanaka T, Moore Z, Salimi S, Sicrra F, de Cabo R: Measuring biological aging in humans: A quest. Aging Cell 2020, 19:e13080.
2. Sen P, Shah P P, Nativio R, Berger S L: Epigenetic Mechanisms of Longevity and Aging. Cell 2016, 166:822-839.
3. Kane A E, Sinclair D A: Epigenetic changes during aging and their reprogramming potential. Critical Reviews in Biochemistry and Molecular Biology 2019, 54:61-83.
4. Zhang W, Qu J. Liu G H, Belmonte J C I: The ageing epigenome and its rejuvenation. Nature Reviews Molecular Cell Biology 2020, 21:137-150.
5. Rando T A, Chang H Y: Aging, rejuvenation, and epigenetic reprogramming: resetting the aging clock. Cell 2012, 148:46-57.
6. López-Otin C, Blasco M A, Partridge L, Serrano M, Kroemer G: The hallmarks of aging. Cell 2013, 153:1194-1217.
7. Bocklandt S, Lin W, Sehl M E, Sanchez F J, Sinsheimer J S, Horvath S, Vilain E: Epigenetic predictor of age. PLoS One 2011, 6:e14821.
8. Garagnani P. Bacalini M G, Pirazzini C. Gori D. Giuliani C, Man D, Di Blasio A M, Gentilini D, Vitale G, Collino S, et al: Methylation of ELOVL2 gene as a new epigenetic marker of age. Aging Cell 2012, 11:1132-1134.
9. Hannum G, Guinney J, Zhao L, Zhang L, Hughes G, Sadda S, Klotzle B, Bibikova M, Fan J B, Gao Y, et al: Genome-wide methylation profiles reveal quantitative views of human aging rates. Mol Cell 2013, 49:359-367.
10. Horvath S: DNA methylation age of human tissues and cell types. Genome Biol 2013, 14:R115.
11. Lin Q. Weidner C I, Costa I G, Marioni R E, Ferreira M R, Deary I J, Wagner W: DNA methylation levels at individual age-associated CpG sites can be indicative for life expectancy. Aging (Albany NY) 2016, 8:394-401.
12. Horvath S, Raj K: DNA methylation-based biomarkers and the epigenetic clock theory of ageing. Nat Rev Genet 2018.
13. Marioni R, Shah S, McRae A. Chen B, Colicino E. Harris S, Gibson J, Henders A. Redmond P, Cox S, et al: DNA methylation age of blood predicts all-cause mortality in later life. Genome Biol 2015, 16:25.
14. Chen B H, Marioni R E, Colicino E. Peters M J, Ward-Caviness C K. Tsai P C, Roetker N S, Just A C, Demerath E W, Guan W, et al: DNA methylation-based measures of biological age: meta-analysis predicting time to death. Aging (Albany NY) 2016, 8:1844-1865.
15. Horvath S. Pirazzini C, Bacalini M G. Gentilini D, Di Blasio A M, Delledonne M, Mari D, Arosio B, Monti D, Passarino G, et al: Decreased epigenetic age of PBMCs from Italian semi-supercentenarians and their offspring. Aging (Albany NY) 2015, 7:1159-1170.
16. Marioni R E, Shah S, McRae A F, Ritchie S J, Muniz-Terrera G, Harris S E, Gibson J, Redmond P. Cox S R, Pattie A, et al: The epigenetic clock is correlated with physical and cognitive fitness in the Lothian Birth Cohort 1936. Int J Epidemiol 2015, 44:1388-1396.
17. Horvath S, Mah V. Lu A T, Woo J S, Choi O W, Jasinska A J, Riancho J A, Tung S. Coles N S, Braun J, et al: The cerebellum ages slowly according to the epigenetic clock. Aging (Albany NY) 2015, 7:294-306.
18. Horvath S, Garagnani P, Bacalini M G, Pirazzini C, Salvioli S, Gentilini D, Di Blasio A M, Giuliani C, Tung S, Vinters H V. Franceschi C: Accelerated epigenetic aging in Down syndrome. Aging Cell 2015, 14:491-495.
19. Horvath S, Levine A J: HIV-1 Infection Accelerates Age According to the Epigenetic Clock. J Infect Dis 2015, 212:1563-1573.
20. Horvath S, Erhart W, Brosch M, Ammerpohl O, von Schonfels W. Ahrens M. Heits N, Bell J T, Tsai P C, Spector T D, et al: Obesity accelerates epigenetic aging of human liver. Proc Natl Acad Sci USA 2014, 111:15538-15543.
21. Levine M E, Lu A T, Chen B H, Hernandez D G. Singleton A B, Ferrucci L. Bandinelli S, Salfati E. Manson J E, Quach A, et al: Menopause accelerates biological aging. Proc Natl Acad Sci USA 2016, 113:9327-9332.
22. Petkovich D A, Podolskiy D I, Lobanov A V, Lee S G, Miller R A, Gladyshev V N: Using DNA Methylation Profiling to Evaluate Biological Age and Longevity Interventions. Cell Metab 2017, 25:954-960 e956.
23. Cole J J, Robertson N A, Rather M I, Thomson J P, McBryan T, Sproul D, Wang T, Brock C, Clark W, Ideker T. et al: Diverse interventions that extend mouse lifespan suppress shared age-associated epigenetic changes at critical gene regulatory regions. Genome Biol 2017, 18:58.
24. Wang T, Tsui B, Kreisberg J F, Robertson N A, Gross A M, Yu M K, Carter H, Brown-Borg H M, Adams P D, Ideker T: Epigenetic aging signatures in mice livers are slowed by dwarfism, caloric restriction and rapamycin treatment. Genome Biol 2017, 18:57.
25. Stubbs T M, Bonder M J, Stark A K, Krueger F, von Meyenn F. Stegle O, Reik W: Multi-tissue DNA methylation age predictor in mouse. Genome Biol 2017, 18:68.
26. Thompson M J, Chwialkowska K, Rubbi L, Lusis A J, Davis R C, Srivastava A. Korstanje R. Churchill G A, Horvath S, Pellegrini M: A multi-tissue full lifespan epigenetic clock for mice. Aging (Albany NY) 2018, 10:2832-2854.
27. Meer M V. Podolskiy D I, Tyshkovskiy A, Gladyshev V N: A whole lifespan mouse multi-tissue DNA methylation clock. eLife 2018, 7:e40675.
28. Fahy G M, Brooke R T, Watson J P, Good Z, Vasanawala S S, Maecker H. Leipold M D, Lin D T S, Kobor M S, Horvath S: Reversal of epigenetic aging and immunosenescent trends in humans. Aging Cell 2019, 18:e13028.
29. Hannum G: Genome-wide methylation profiles reveal quantitative views of human aging rates. Mol Cell 2013, 49.
30. Lin Q. Weidner C I, Costa I G, Marioni R E, Ferreira M R P, Deary I J: DNA methylation levels at individual age-associated CpG sites can be indicative for life expectancy. Aging 2016, 8:394-401.
31. Horvath S: DNA methylation age of human tissues and cell types. Genome Biol 2013. 14.
32. Horvath S. Oshima J, Martin G M, Lu A T, Quach A. Cohen H, Felton S, Matsuyama M, Lowe D. Kabacik S, et al: Epigenetic clock for skin and blood cells applied to Hutchinson Gilford Progeria Syndrome and ex vivo studies. Aging (Albany NY) 2018. 10:1758-1775.
33. The Bc, Bock C. Halbritter F, Carmona F J, Tierling S, Datlinger P, Assenov Y, Berdasco M, Bergmann A K, Booher K, et al: Quantitative comparison of DNA methylation assays for biomarker development and clinical applications. Nature Biotechnology 2016, 34:726.
34. Jylhava J. Pedersen N L, Hagg S: Biological Age Predictors. EBioMedicine 2017, 21:29-36.
35. Horvath S. Raj K: DNA methylation-based biomarkers and the epigenetic clock theory of ageing. Nat Rev Genet 2018. 19:371-384.
36. Morgello S. Gelman B, Kozlowski P, Vinters H. Masliah E. Comford M, Cavert W, Marra C. Grant I, Singer E: The National NeuroAIDS Tissue Consortium: a new paradigm in brain banking with an emphasis on infectious disease. Neuropathol Appl Neurobiol 2001, 27:326-335.
37. Horvath S, Stein D J, Phillips N, Heany S J, Kobor M S, Lin D T S, Myer L, Zar H J, Levine A J, Hoare J: Perinatally acquired HIV infection accelerates epigenetic aging in South African adolescents. AIDS (London, England) 2018, 32:1465-1474.
38. Kabacik S, Horvath S, Cohen H, Raj K: Epigenetic ageing is distinct from senescence-mediated ageing and is not prevented by telomerase expression. Aging (Albany NY) 2018, 10:2800-2815.
39. Friedman J. Hastie T, Tibshirani R Regularization Paths for Generalized Linear Models via Coordinate Descent. Journal of Statistical Software 2010, 33:1-22.
40. Bocklandt S, Lin W. Sehl M E, Sanchez F J, Sinsheimer J S, Horvath S, Vilain E: Epigenetic predictor of age. PLoS One 2011, 6.
41. Weidner C I: Aging of blood can be tracked by DNA methylation changes at just three CpG sites. Genome Biol 2014, 15.
42. Christiansen L, Lenart A, Tan Q, Vaupel J W, Aviv A, McGue M, Christensen K: DNA methylation age is associated with mortality in a longitudinal Danish twin study. Aging Cell 2015.
43. Pema L. Zhang Y. Mons U, Holleczek B. Saum K U, Brenner H: Epigenetic age acceleration predicts cancer, cardiovascular, and all-cause mortality in a German case cohort. Clinical Epigenetics 2016, 8:1-7.

### Example 3: Universal DNA Methylation Age Across Mammalian Tissues

The references cited in this Example are found at the end of this Example.

Aging is often perceived as a degenerative process caused by random accrual of cellular damage over time. In spite of this, age can be accurately estimated by epigenetic clocks based on DNA methylation profiles from almost any tissue of the body. Since such pan-tissue epigenetic clocks have been successfully developed for several different species, it is difficult to ignore the likelihood that a defined and shared mechanism instead, underlies the aging process. To address this, we generated 10,000 methylation arrays, each profiling up to 37,000 cytosines in highly-conserved stretches of DNA, from over 59 tissue-types derived from 128 mammalian species. From these, we identified and characterized specific cytosines, whose methylation levels change with age across mammalian species. Genes associated with these cytosines are greatly enriched in mammalian developmental processes and implicated in age-associated diseases. From the methylation profiles of these age-related cytosines, we successfully constructed three highly accurate universal mammalian clocks. The universal clocks for similarly accurate for estimating ages (r>0.95) of any mammalian species and tissue with a single mathematical formula.

Collectively, these new observations support the notion that aging is indeed evolutionarily conserved and coupled to developmental processes across all mammalian species—a notion that was long-debated without the benefit of this new and compelling evidence.

Aging is associated with multiple cellular changes that are often tissue-specific. Cytosine methylation however, is unusual in this regard as it is strongly correlated with age across virtually all tissues. This feature can be capitalized upon to develop multivariate age-estimators (pan-tissue epigenetic clocks) that are applicable to most or all tissues of a species. This approach produced the first human pan-tissue clock that was based on 353 age-related CpGs 1. Subsequent successes in developing similar pan-tissue clocks for other species hint at the universality of the aging process. To investigate this, we sought to i) identify and characterize cytosines whose methylation levels change with age in all mammals and ii) develop universal age-estimators that apply to all mammalian species and tissues (universal epigenetic clocks for mammals). Towards these ends, we employed a novel Infinium array (HorvathMammalMethylChip40) that profiles methylation levels of up to 37k CpGs with flanking DNA sequences that are highly-conserved across the mammalian class 2. We obtained such profiles from almost 10,000 samples from 59 tissue types, derived from 128 mammalian species, representing 15 phylogenetic orders with age ranging from prenatal, to 139-years-old (bowhead whale). The species tested had maximum life spans from 3.8 to 211 years and adult weights from 0.004 to 100,000 kilograms.

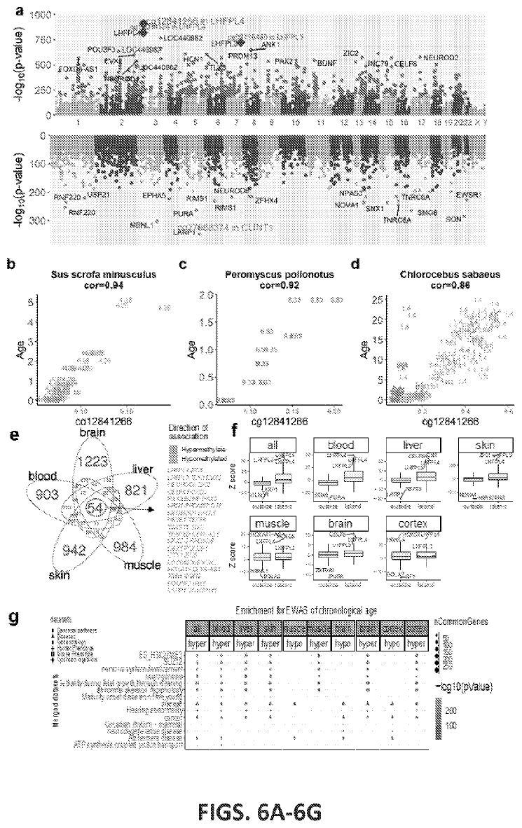

To identify age-related CpGs, we carried out two-stage meta-analysis across species and tissues. Cytosines that become increasingly methylated with age (i.e., positively correlated) were found to be more highly conserved (FIG. 6a). From these, we identified 665 age-related CpGs, within a threshold significance of α=10-200 across all eutherian species and tissues (FIG. 6a). Cytosines cg12841266 (P=6.2×10-908) and cg11084334 (P=2.0×10-823), located in exon 2 of the LHFPL4 gene were the most predictive across all species, having a correlation>0.8 in 24 species, of which three are shown in FIG. 6b-d. Another highly-correlated cytosine, cg09710440, resides in LHFPL3 (P=5.1×10-724), a paralog of LHFPL4 (FIG. 6a). As LHFPL4 and LHFPL3 are in human chromosomes 2 and 7 respectively, their age-related gain of methylation is unlikely to be random. It implies instead their involvement in the aging process, even if their activities as nucleators of GABA receptors do not immediately conjure an obvious mechanism. Indeed, methylation of LHFPL4 cg12841266 was strongly correlated with age of multiple mouse tissues in both development (r=0.58 and P=8.9×10-11) and post-development stages (r=0.45 and P=2.3×10-76), particularly in the brain (r=0.92 and P=6.95×10-8), muscle (r=0.89 and P=7.6×10-7), liver (r=0.79 and P=1.9×10-117), and blood (r=0.89 and P=1.0×10-53). Consistent with increased methylation, expression of both LHFPL4 and LHFPL3 declines with increasing age in numerous, albeit not all, human and mouse tissues. In particular, their reduced expression is consistently observed in the brain 3, 4. Importantly, age-related methylation changes in young animals concur strongly with those observed in middle-aged or old animals, excluding the likelihood that the changes are those involved purely in the process of organismal development.

### Meta-Analysis of Age-Related CpGs Across Specific Tissues

To gain a wider and deeper understanding of age-related CpGs within specific tissues across different species, we focused on 5 organs: brain (whole and cortex), blood, liver, muscle and skin. We performed EWAS meta-analysis on 851 whole brains (17 species), 391 cortices (6 species), 3552 blood (28 species), 1221 liver (9 species), 345 muscle (5 species), and 1452 skin (31 species). Consistently across all tissues, there were more CpG with positive correlations with age than negative ones and most of them were located within CpG islands, which are known to become increasingly methylated with age (FIG. 6f). While many of these cytosines were either specific to individual organs or shared between several organs, 54 potential universal age-related CpGs were shared among all the five organs (FIG. 6e). Strikingly, the overwhelming majority of the 40 genes that are proximal to these 54 CpGs encode transcription factors with homeobox domain, and are involved in developmental processes.

### Functional Enrichment Analysis of Age-Related CpGs

We employed a pathway enrichment tool (GREAT hypergeometric test based on genomic regions5) to analyze the top 1,000 positively and 1,000 negatively correlated age-related CpGs and their proximal genes in all tissues, individually or collectively, to ascertain whether they are associated with particular biological processes or cellular pathways (FIG. 6g). We demonstrated that our enrichment results are not confounded by the special design of the mammalian methylation array. From positively-correlated CpGs across all tissues, the most enriched (P=3.7×10-207) Gene Ontology term was “nervous system development”, which also appeared prominently in blood (P=4.7×10-230), liver (P=7.6×10-136), muscle (P=1.4×10-12), skin (P=5.4×10-141), brain (P=1.0×10-42) and cortex (P=7.5×10-80). Other top-scoring terms include “pattern specification” and “anatomical structure development”. Evidently, many hypermethylated age-related CpGs in all the five organs may be proximal to development genes. At the molecular level, many of these CpGs are in positions targeted by SUZ12, which is one of the core subunits of polycomb repressive complex 2 (all tissue P=7.1×10-225, blood P=3.9×10-259, liver P=1.7×10-149, muscle P=8.2×10-16, skin P=2.6×10-150, brain P=8.7×10-54, and cerebral cortex P=6.1×10-87); echoing previous human EWAS studies6, 7 EED, another core subunit of PRC2, shows similarly high significant P-values, e.g. P=1.7×10-262 in all tissues. Strong enrichment can also be found in promoters with H3K27me3 modification. These were observed in all tissue (P=2.8×10-266), blood (P=3.9×10-283), liver (P=3.3×10-189), muscle (P=8.7×10-18), skin (P=3.3×10-189), brain (P=3.3×10-68), and cortex (P=5.1×10-116). These results reinforce the association between development and aging.

This may appear counterintuitive but finds support from the fact that mice with compromised development following ablation of growth hormone receptors (GHRKO), exhibit significant slowing down of their aging process 8. We demonstrated that the universal epigenetic clocks are slowed in cortex, liver, and kidneys from GHRKO mice. Interestingly, although there were 3,617 enrichments of hypermethylated age-related CpGs across all tissues, only 12 were found for hypomethylated ones. The apparent scarcity of the latter is particularly strong in skin, blood, and liver. However, this is not the case for the brain, cortex, and muscle, where there was instead greater enrichment of hypomethylated age-related cytosines; a trend that seemingly parallels the rate of tissue turn-over. The cytosines that were negatively associated with age in brain (P=9.0×10-18, cortex (P=4.0×10-19) and muscle (P=2.5×10-4), are enriched in the circadian rhythm pathway, indicating that besides commonly shared processes of development, which is universally implicated in aging of all tissues, organ-specific ones are also clearly in operation.

Another relevant observation is the enrichment of negative age-related cytosines in an up-regulated gene set in Alzheimer's disease. This was observed in the whole brain (P=2.1×10-30), the cortex (P=5.9×10-22), and in muscle tissue (P=2.5×10-5). Although this gene set was also enriched in blood (P=1.5×10-6) and all tissues combined (P=1.4×10-4), it was associated with positive age-related CpGs instead indicating that some age-related gene sets can be impacted by negative and positive age-related CpGs, potentially influencing different members of the set or perhaps having opposing transcriptional outcomes resulting from methylation. Another highly-relevant example of this is the observation concerning mitochondrial function. While hypomethylated age-related cytosines in brain, cortex, and muscle are enriched for numerous mitochondria-related genes; in blood and skin, however, these are enriched for positive age-related cytosines.

Overlap of Age-Related Cytosines with Human Traits and Diseases

To uncover potential correlation between age-related cytosines and known human traits, the proximal genomic regions of the same top 1,000 positively and 1,000 negatively associated CpGs were overlaid with the top 5% of genes that were associated with numerous human traits identified by GWAS. At threshold of P<5.0×10-4, overlaps were found with genes associated with longevity, Alzheimer's, Parkinson's and Huntington's disease, dementia, epigenetic age acceleration, age at menarche, leukocyte telomere length, inflammation, mother's longevity, metabolic diseases, obesity (fat distribution, body-mass index), etc.; many of which are associated with advancing age.

### Development of Universal Pan-Tissue Epigenetic Clocks of Age Across Mammals

Having identified age-related cytosines shared across mammalian species and tissues, we proceeded to use them to develop universal mammalian epigenetic age clocks. We developed three universal mammalian age-estimators, which differ with respect to output. The first, universal naïve clock (Clock 1) directly correlates DNA methylation profile to chronological age. To allow biologically meaningful comparisons between species with very different life-spans, we developed a second universal clock that defines individual age relative to the maximum lifespan of its species: generating relative age estimates between 0 and 1. As the accuracy of this universal relative age clock (Clock 2) could be compromised in species for which knowledge of maximum lifespan is unavailable, a third universal clock was developed, which omits maximum lifespan and uses instead average age at sexual maturity. Age at sexual maturity was chosen as species characteristics since it correlates strongly with maximum lifespan on the log scale (Pearson correlation r=0.82, p=6×10-183 across all mammalian species in AnAge). This third clock is referred to as the universal log-linear transformed age clock (Clock 3).

### Performance of Universal Epigenetic Clocks Across Species

We employed two different strategies for evaluating the accuracy of the clocks. First, leave-one-fraction-out (LOFO) cross-validation analysis randomly divided the data set into 10 fractions, each of which contained the same proportions of species and tissue types, and a different fraction is left out for validation at each iteration of analysis. Second, leave-one-species-out analysis (LOSO) was similarly cross-validated with the omission of a species at each iteration.

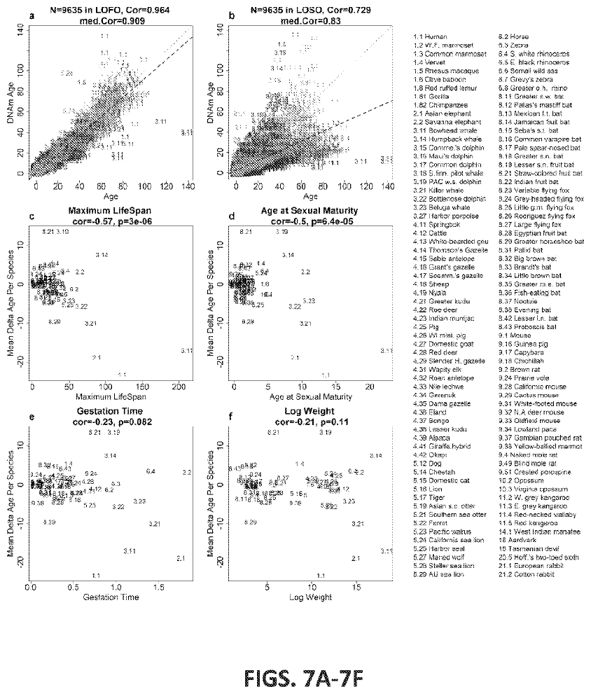

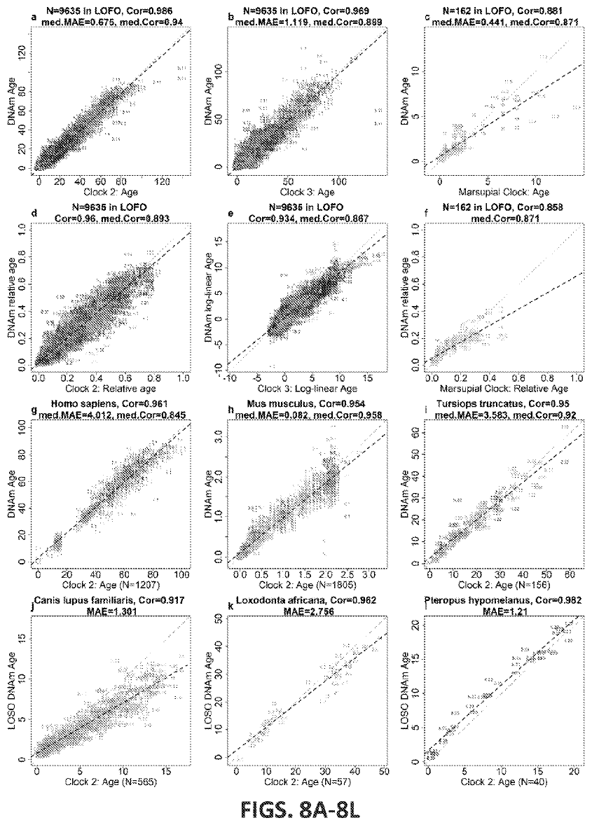

According to LOFO cross-validation, the epigenetic clocks were remarkably accurate (r>0.96), with a median error of less than 1 year and a median relative error of less 3.7 percent (FIGS. 7a, 8a-b). According to the LOSO evaluation, the clocks reached age correlations up to r=0.94. The median correlation (and MAE) across species was as strong with either LOFO or LOSO evaluations. For some species such as bowhead whales, however, epigenetic age as predicted by the naïve clock accords poorly with chronological age (FIG. 7b). We investigated and ascertained that the mean difference between LOSO DNAmAge and chronological age is negatively correlated with maximum lifespan (r=−0.57, p=3×10-6) and age at sexual maturity (r=−0.5, p=6.4×10-5) of the species (FIG. 7c-d). Here, the strength of clock 2 comes to fore as it is not affected by maximum lifespan, which was incorporated into it during its construction. Clock 2 and clock 3 achieve a correlation of r=0.96 and r=0.93 between DNAm and observed transformed age, respectively (FIG. 8d,e). Both of these clocks present comparably accurate LOFO estimates in numerous tissue types in 58 species, with a representation in FIG. 8g-i of LOFO Clock 2 correlations for humans (r=0.961, 19 tissues), mice (r=0.954, 25 tissues), and bottlenose dolphins (r=0.95, 2 tissues). While the clock accurately predicted the age for one mysticete species, the humpback whale and all other mammalian species, the ages of bowhead samples were sometimes underestimated (species index 3.11 in FIG. 5a, b). This may simply reflect the inaccuracy of the age estimations used for bowhead whales, which were aged using the aspartic acid racemization rate. These clocks are similarly accurate with LOSO age-estimates between evolutionarily distant species including dogs (r=0.917, MAE=1.3), Savanna elephants (r=0.962, MAE<3 years), and flying foxes (r=0.982, MAE=1.2) (FIG. 8j-l). Such high predictive accuracy of LOSO analysis demonstrates that these universal clocks are applicable to mammalian species that are not part of the training data. The three universal clocks performed just as well in 63 species, for which there were fewer than 15 samples (r˜0.9, MAE˜1 year), showing very strong correlation between estimated and actual relative age (r=0.92).

With regards to marsupials, we encountered two limitations. First, less than half of the eutherian CpGs apply to marsupials 2. Second, there were only seven marsupial species in our data set with total sample size N=162. These limitations notwithstanding, we were still able to construct a fourth universal clock for estimating relative age in marsupials (age correlation, r=0.88, med.Cor=0.87 in FIG. 8c,f).

### Performance of Universal Epigenetic Clocks Across Tissues

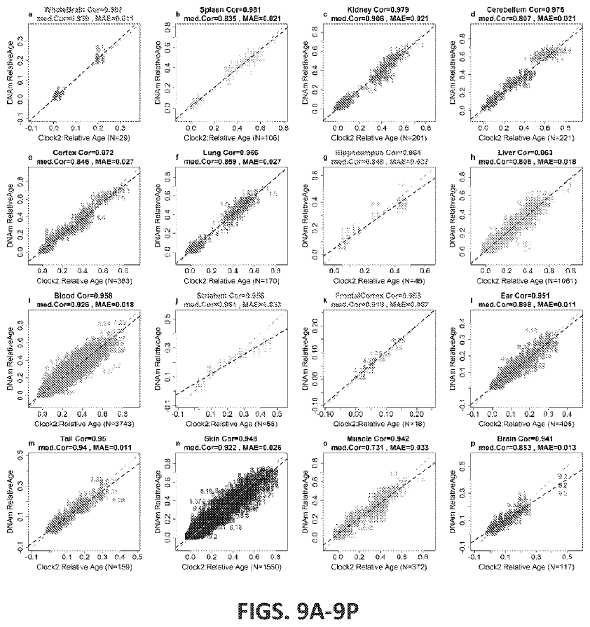

As the epigenome landscape vanes markedly across tissue types 9, 10, we assessed tissue-specific accuracy of clock 2 for relative age (r=0.96, FIG. 8d). Of the 33 distinct tissue types, the median correlation is 0.94 and median MAE for relative age is 0.026. There was high age-correlation with whole brain (r=0.987), cortex (r=0.972), hippocampus (r=t 964), striatum (r=0.956), cerebellum (r=0.975), spleen (r=0.981), and kidney (r=0.979) (FIG. 9). Blood and skin also exhibited similarly high estimates of relative age correlations across different species: blood (r=0.958, MAE=0.018, 74 species) and skin (r=0.948, MAE=0.026, 56 species) (FIG. 9i,n).

The universality of aging across all mammalian species has engendered speculations of its cause, with the predominant notion that random damage to cellular constituents underlies this process. The ability to accurately estimate ages of mammals by virtue of their methylation profiles however, introduces the likelihood of a largely deterministic process.

We investigated this question by generating an unprecedentedly large set of DNA methylation profiles from over 121 eutherian species and 7 marsupial species, from which an unambiguous feature emerged. Genes that are proximal to age-related CpGs, overwhelmingly represent those involved in the process of development, such as HOX and PAX. This is consistent with enrichment of these cytosines in target sites of PRC2 and bivalent chromatin domains, which control expression of HOX and other developmental genes in all vertebrates and beyond. It appears therefore, that aging is hard-wired into life through processes associated with development.

A large body of literature connects growth/development to aging starting with the seminal work by Williams 1957 11 and a recent study by de Magalhacs12. This connection is also apparent when Yamanaka factor-mediated reversion of adult cells to embryonic stem cells is accompanied by resetting of their age to prenatal epigenetic age, matching their development stage 1. Therefore, methylation regulation of the genes involved in development (during and after the developmental period) may constitute a key mechanism linking growth and aging. The universal epigenetic clocks demonstrate that aging and development are coupled and share important mechanistic processes that operate over the entire lifespan of an organism.

Other notable age-related genes and processes that were uncovered include LHFPL4 and LHFPL3 whose reported function in synaptic clustering of GABA receptors does not immediately present an obvious connection to aging across all tissues. However, the extremely strong correlation of CpGs near these paralogues with age argues strongly for their role in the aging process. The LARP1 gene ranks first in liver and second across all tissues for hypomethylation with age and encodes a protein that regulates translation of downstream targets of mTOR13, which has very well-documented links with aging and longevity. The implication of circadian rhythm genes, exclusively in aging brain tissues, reveals tissue-specific changes that occur in parallel with universal developmental ones. Furthermore, involvement of circadian rhythm genes in aging echoes recent observations in mice 4.

The implication of multiple genes related to mitochondrial function supports the long-argued importance of this organelle in the aging process. It is also important to note that many of the identified genes are implicated in a host of age-related pathologies and conditions, bolstering the likelihood of their active participation in, as opposed to passive association with, the aging process.

Future elucidation of how development is mechanistically connected to aging will be aided by the universal mammalian clocks. The leave-one-species-out cross validation analysis demonstrates that these clocks generalize very well to mammalian species that were not part of the training set. The ability to construct universal mammalian epigenetic clocks that can accurately predict the age of animals and tissues that were not part of the training set fulfils Popper's dictum of falsifiability, which requires that a theory make testable predictions on the basis of which it can be refuted. The epigenetic clocks presented here, built on the universality of mammalian aging, pass this test with remarkable ease and accuracy.

### Methods

### Tissue Samples

### Quality Controls for Establishing Universal Clocks

We generated two variables to guide the quality control (QC) of the study samples: the first being a variable indicating the confidence (0 to 100%) in the chronological age estimate of the sample. For example, a low confidence was assigned to samples from wild animals whose ages were estimated based on body length measurements. The epigenetic clocks were trained and evaluated in tissue samples whose confidence exceeded 90% (>=90%). The second quality control variable was an indicator variable (yes/no) that flagged technical outliers or malignant (cancer) tissue. Since we were interested in “normal” aging patterns we excluded tissues from preclinical studies surrounding anti-aging or pro-aging interventions.

### Species Characteristics

Species characteristics such as maximum lifespan (maximum observed age), age at sexual maturity, and gestational length were obtained from an updated version of the Animal Aging and Longevity Database 14 (AnAge, http://genomics.senescence.info/help.html#anage).

### Meta Analysis for EWAS of Age

We carried out two methods to combine EWAS results across species and tissues, as described below.

Two-Stage Meta Analysis in Conjunction with Stouffer's Method

Our meta analysis of age combined correlation test statistics calculated in 133 different species-tissue strata (from 58 species) with a minimal sample size of 15 (N≥15). In the first stage, we combined the EWAS results across tissues within the same species to form species specific meta-EWAS results. In the second stage, we combined the total of 58 species EWAS results to form a final meta-EWAS of age. All the meta analyses in both stages were performed by the unweighted Stouffer's method, as conducted in METAL15.

### Stratification of Age Groups

To assess whether the age related CpGs in young animals relate to those in old animals, we split the data into 3 age groups: young age (age<1.5*age at sexual maturity, ASM), middle age (age between 1.5 and 3.5 ASM), and old age group (age≥3.5 ASM). The threshold of sample size in species-tissue was relaxed to N≥10. The age correlations in each age group were meta analyzed using the above mentioned two-stage meta analysis approach.

### Brain EWAS

Analogously, we applied the two approaches to brain EWAS results; more than 900 brain tissues from human, vervet monkey, mice, olive bamboo, brown rat, and pig species across cerebellum, cortex, hippocampus, hypothalamus, striatum, subventricular zone (SVZ), and whole brain.

### EWAS of Single Tissue

One-stage unweighted Stouffer's method and Median Z score were also applied to EWAS results from cerebellum and cortex, respectively. Similarly, we carried out meta-analysis EWAS of blood, liver, muscle, and skin. Blood EWAS results were combined across 7 families including 367 tissues from humans, 565 from dogs, 170 from mice, 36 from killer whales, 137 from bottlenose dolphins, 83 from Asian elephants, etc. Skin EWAS results were combined across 5 families including 95 from bowhead whales, 638 tissues from 19 bat species, 180 from killer whales, 105 from naked mole rats, 72 from humans, etc. Liver EWAS results were combined across four families including 583 mice, 97 from humans, 48 from horses, etc. Muscle EWAS results were combined across four families including 24 from evening bats, 57 from humans, and 19 from naked mole rats, etc. Cerebellum EWAS results were combined across Primates and Rodentia including 46 from humans. Another 46 cerebral cortex tissues profiled in the same human individuals were included in the cortex EWAS, in which the meta analysis was also combined across Primates, Rodentia, and a third Order: 16 pigs from Artiodactyla. 5.

We used the R gmirror function to depict mirror image Manhattan plots.

### GREAT Analysis

We applied the GREAT analysis software tool6 to the top 1000 hypermethylated and the top 1000 hypomethylated CpGs from EWAS of age. GREAT implemented foreground/background hypergeometric tests over genomic regions where we input all of the 37k CpG regions of our mammalian array as background and the genomic regions of the 1000 CpGs as foreground. This yielded hypergeometric p-values not confounded by the number of CpGs within a gene. We performed the enrichment based on the settings (assembly: Hg19, Proximal: 5.0 kb upstream, 1.0 kb downstream, plus Distal: up to 50 kb) for about 76,290 gene sets associated with GO terms, MSigDB (including gene sets for upstream regulators), PANTHER, KEGG pathway, disease ontology, gene ontology, human and mouse phenotypes. We report the gene sets with FDR<0.05 and list nominal hypergeometric P-values, FDR and Bonferroni corrected P-values.

### EWAS-GWAS Based Overlap Analysis

Our EWAS-GWAS based overlap analysis related the gene sets found by our EWAS of age with the gene sets found by published large-scale GWAS of various phenotypes, across body fat distribution, lipid panel outcomes, metabolic outcomes, neurological diseases, six DNAm based biomarkers, and other age-related traits. A total of 69 GWAS results were studied. The six DNAm biomarkers included four epigenetic age acceleration measures derived from 1) Horvath's pan-tissue epigenetic age adjusted for age-related blood cell counts referred to as intrinsic epigenetic age acceleration (IEAA) 1, 16, 2) Hannum's blood-based DNAm age17; 3) DNAmPhenoAge 18; and 4) the mortality risk estimator DNAmGrimAge 19, along with DNAm based estimates of blood cell counts and plasminogen activator inhibitor 1 (PAI1) levels19. For each GWAS result, we used the MAGENTA software to calculate an overall GWAS P-value per gene, which is based on the most significant SNP association P-value within the gene boundary (+/−50 kb) adjusted for gene size, number of SNPs per kb, and other potential confounders 20. We pruned in the genomic regions of GWAS genes present in the mammalian array. For each EWAS results, we studied the genomic regions from the top 1000 CpGs hypermethylated and hypomethylated with age, respectively. To assess the overlap with a test trait, we selected the top 5% genes for each GWAS trait and calculated one-sided hypergeometric P values based on genomic regions (as detailed in 21, 22). The number of background genomic regions in the hypergeometric test was based on the overlap between the entire genes in a GWAS and the entire genomic regions in our mammalian array. We highlighted the GWAS trait when its hypergeometric P value reached 5×10-4 with EWAS of age in any tissue type.

Association of LHFPL gene expression with chronological age in human and mouse To study if LHFPL4 or LHFPL3 play a role in age-related transcriptional changes surrounding nearby genes, we analyzed several transcriptomic data across multiple tissues and species. In humans, our analysis leveraged gene expression studies from 1) GTEx project, 2) two gene expression data studied in 21 (GEO datasets from studies 23, 24) and 3) the summary data across three studies in Isildak et al.³ for investigating age-related brain expression in developmental (age<20) and aging (age≥20) periods. In mice, we analyzed the summary data from The Tabula Muris Consortium 4 which generated single cell RNA seq data from 23 mouse tissues across the lifespan.

### Three Universal Mammalian Clocks for Eutherians

We applied elastic net regression models to establish three universal mammalian clocks for estimating chronological age across all tissues in eutherians. The three elastic net regression models corresponded to different outcome measures described in the following: 1) log transformed chronological age: log(Age+2) where an offset of 2 years was added to avoid negative numbers in case of prenatal samples, 2) −log(−log(RelativeAge)) and 3) log-linear transformed age. DNAm age estimates of each clock were computed via the respective inverse transformation. Age transformations used for building universal clocks 2-4 incorporated three species characteristics: gestational time (GT), age at sexual maturity (ASM), and maximum lifespan (maxAge). All of these species variables surrounding time are measured in units of years.

### Loglog Transformation of Relative Age for Clock 2

Our measure of relative age leverages gestation time (GestationT) and maximum lifespan. We define relative age (RelativeAge) and apply Log-log transformation as the following:

$$RelativeAge = \frac{Age + GestationT}{MaxAge + GestationT} \qquad ( 1 )$$

$$LoglogAge = - log ( - log ( RealtiveAge ) ) \qquad ( 2 )$$

By definition, RelativeAge is between 0 to 1 and loglogAge is positively correlated with age. Universal clock 2 predicts LoglogAge and next applies an inverse transformation to estimate DNAmAge:

$$DNAmAge = exp ( - exp ( - LoglogAge ) ) * ( MaxAge + GestationT ) - GestationT \qquad ( 3 )$$

All species characteristics (e.g. MaxAge, gestational time) come from our updated version of AnAge. We were concerned that the uneven evidence surrounding the maximum age of different species could bias our analysis. While billions of people have been evaluated for estimating the maximum age of humans (122.5 years), the same cannot be said for any other species. To address this concern, we made the following admittedly ad-hoc assumption: the true maximum age of non-human species is 30% higher than that reported in AnAge. Therefore, we multiplied the reported maximum lifespan of non-human species by 1.3. Our predictive models turn out to be highly robust with respect to this assumption (data not shown).

### Transformation Based on Log-Linear Age for Clock 3

$$y = f ( x ) = \{ \begin{matrix}x - 1 , x \text{?} 1 \\ log ( x ) , x < 1\end{matrix} \qquad ( 5 )$$

$${f}^{- 1} ( y ) = \{ \begin{matrix}y + 1 , y \geq 0 \\ exp ( y ) , y < 0\end{matrix} \qquad ( 6 )$$

$$\text{?} \text{indicates text missing or illegible when filed}$$

This transformation ensures continuity and smoothness at the change point x=1. In our study, the argument x is the ratio RelativeAdultAge.

(6)

### Elastic Net Regression

We applied the elastic net regression models to train all samples that selected 1000 to 2000 CpGs for clocks 1-3 and 30 CpGs for the marsupial clock. To assess the accuracy of the elastic net regression models, we used leave-one-fraction-out (LOFO) and leave one-species-out (LOSO) cross validation. In LOFO, we randomly split the entire dataset into 10 fractions each of which had the same distribution in species and tissue types. Each penalized regression model was trained in 9 fractions but evaluated in the 10th left out fraction. After circling through the 10 fractions, we arrived at LOFO predictions which were subsequently related to the actual values.

The LOSO cross validation approach trained each model on all but one species. The left out species was used a test set. The LOSO approach was used to assess how well the penalized regression models generalize to species that were not part of the training data. To ensure unbiased estimates of accuracy, all aspects of the model fitting (including pre-filtering of the CpG) were only conducted in the training data in both LOFO and LOSO analysis. Elastic net regression in the training data was implemented by setting the glmnet model parameter alpha to 0.5. Ten-fold cross validation in the training data was used to estimate the tuning parameter lambda. For computational reasons, we fitted the glmnet model to the top 4000 CpGs with the most significant median Z score (age correlation test) in the training data. To accommodate different samples sizes of the species we used weighted regression as needed where the weight was the inverse of square root of species frequency or 1/20 (whichever was higher). The final versions of the different universal clocks used all available data.

### Statistics for Performance of Model Prediction

To validate our model, we used DNAm age estimates from LOFO and LOSO analysis, respectively. At each type of estimates, we performed Pearson correlation coefficients and computed median absolute error (MAE) between DNAm based and observed variables across all samples. Correlation and MAE were also computed at species level, limited to the subgroup with samples N≥=15 (within a species or within a species-tissue category). We reported the medians for the correlation estimates (med.Cor) and the medians for the MAE estimates (med.MAE) across species, respectively. Analogously, we repeated the same analysis at species-tissue level, limited to the subgroup with sampleN>=15 (within a specie-tissue category).

### Covariates and Coefficient Values

The universal mammalian clock for relative age is based on 783 CpGs whose coefficient values are specified in the column “Coef.UniversalRelativeAge”.

The universal mammalian clock for log linear age is based on 724 CpGs whose coefficient values are specified in the column “Coef.UniversalLogLinearAge”. The DNAm Age estimate is estimated in two steps.

First, one forms a weighted linear combination of the CpGs.

The table reports the probe identifier (cg number) used in the custom Infinium array (HorvathMammalMethylChip40). The weights used in this linear combination can be specified in the respective column entitled “Coef.”. The formula assumes that the DNA methylation data measure “beta” values but the formula could be adapted to other ways of generating DNA methylation data.

Second, the weighted average of the CpGs is transformed using a monotonically increasing function F so that it is in units of years.

DNAmAge=F(WeightedAverage).

For universal clock 2 for relative age, the function F is given by the following R code

```
F=function(y,y.maxAge,y.gestation){
  x0=exp(−exp(−y))
  x1=x0*(y.maxAge+y.gestation)
  x=x1−y.gestation
  x
}
```

For universal clock 3 for log linear age, the function F is given by the following R code

$$F = function ( y . pred , \text{} m \text{} 1 ) \text{} \{ ifelse ( \text{} y . pred < 0 , \text{} ( exp ( y . pred ) - 1 ) * m 1 + m 1 , y . pred * m 1 + m 1 ) \}$$

Availability of the epigenetic maximum lifespan predictor will usher in a new era of interventional studies that aim to extend the maximum lifespan of a species as a whole.

Novel “biomarkers of aging”, i.e. assessments that allow one to measure age, are interesting to gerontologists (aging researchers), anti-aging researchers, pharmaceutical companies that carry out preclinical studies.

Finally, this measure may be another component of other molecular biomarkers of aging.

In summary, the invention provides novel epigenetic biomarker of aging. While these DNAm based biomarkers will probably not replace traditional biomarker assessments, they provide complementary information that adds valuable information, with clinical/preclinical applications.

### REFERENCES

1. Bocklandt S, Lin W, Sehl M E, Sanchez F J, Sinsheimer J S, Horvath S, Vilain E: Epigenetic predictor of age. PLoS One 2011, 6.
2. Weidner C I: Aging of blood can be tracked by DNA methylation changes at just three CpG sites. Genome Biol 2014, 15.
3. Horvath S: DNA methylation age of human tissues and cell types. Genome Biol 2013, 14:R115.
4. Hannum G, Guinney J, Zhao L, Zhang L, Hughes G. Sadda S, Klotzle B, Bibikova M, Fan J B, Gao Y, et al: Genome-wide methylation profiles reveal quantitative views of human aging rates. Mol Cell 2013, 49:359-367.
5. Hannum G: Genome-wide methylation profiles reveal quantitative views of human aging rates. Mol Cell 2013, 49.
6. Lin Q, Weidner C I, Costa I G, Marioni R E, Ferreira M R P, Deary I J: DNA methylation levels at individual age-associated CpG sites can be indicative for life expectancy. Aging 2016, 8:394-401.
7. Marioni R, Shah S, McRae A, Chen B, Colicino E. Harris S, Gibson J, Henders A. Redmond P, Cox S, et al: DNA methylation age of blood predicts all-cause mortality in later life. Genome Biol 2015, 16:25.
8. Christiansen L, Lenart A. Tan Q, Vaupel J W, Aviv A, McGue M. Christensen K: DNA methylation age is associated with mortality in a longitudinal Danish twin study. Aging Cell 2015.
9. Pema L, Zhang Y, Mons U. Holleczek B, Saum K U, Brenner H: Epigenetic age acceleration predicts cancer, cardiovascular, and all-cause mortality in a German case cohort. Clinical Epigenetics 2016, 8:1-7.

### Example 4: Epigenetic Clocks for Mice and Humans

The references cited in this Example are found at the end of this Example.

Age can be accurately estimated by epigenetic clocks based on DNA methylation profiles from almost any tissue of the body. Such pan-tissue epigenetic clocks have been successfully developed for several different species including mice. However, it is not yet known whether one can develop an epigenetic clock for mice that also applies to humans.

Here we present a dual species epigenetic clock for mice and humans based on methylation levels in cytosines that are highly conserved in mammals. The human mouse epigenetic clock allows one to estimate the age based on human or mouse DNA with a single mathematical formula.

A critical step toward crossing the species barrier was the use of a mammalian DNA methylation array that profiled 36 thousand probes that were highly conserved across numerous mammalian species (1).

We expect that the availability of these clocks and their impressive performance will provide a significant boost to the attractiveness of the mouse as biological model in aging research. The fact this epigenetic biomarker applies to both species greatly increases the likelihood that findings from preclinical studies in mice will translate to humans.

Beyond their utility, these clocks reveal several salient features with regards to the biology of aging. First, the mouse pan-tissue clock re-affirms the implication of the human pan-tissue clock, which is that aging might be a coordinated biological process that is harmonized throughout the body. Second, the ability to combine these two pan-tissue clocks into a single human-mouse pan-tissue clock attests to the high conservation of the aging process across two evolutionary distant species. Thus a treatment that alters the epigenetic age of mice, as measured using the human-mouse clock is likely to exert similar effects in humans.

The incorporation of two species with very different lifespans such as mouse and human, raises the inevitable challenge of unequal distribution of data points along the age range. This challenge is addressed by the generation of the human-mouse pan-tissue relative age clock which embeds the estimated age in context of the maximal lifespan recorded for the of the relevant species.

The development of the mouse epigenetic clocks described here was based on novel DNA methylation data that were derived from many different mouse tissue types.

We also present clocks for mouse liver, blood, cerebral cortex, fibroblasts, skin, and tails.

### General Background DNA Methylation Based Biomarkers

DNA based biomarker that changes with age is DNA methylation; specifically of cytosine residues of cytosine-phosphate-guanine dinucleotides (CpGs). Machine teaming-based analyses of these changes generated algorithms, known as epigenetic clocks that use specific CpG methylation levels to accurately estimate age that is referred to as DNA methylation age (DNAm age)(2-5).

DNAm aging assays are already highly robust and ready for biomarker development; as reported by the BLUEPRINT consortium (6). DNAm based biomarkers are highly promising molecular biomarkers of aging (7, 8).

Human epigenetic clocks for humans have found many biomedical applications including the measure of age in human clinical trials (9, 10). These clocks provide an estimate of chronological age. The divergence between epigenetic age and calendar age, referred to as epigenetic age acceleration, is associated with increased risk of a host of conditions and pathologies, indicating that epigenetic clocks are associated with biological age. This instigated development of similar clocks for animals including mice. Indeed, numerous mouse epigenetic clocks have since been developed and successfully validated against factors, such as rapamycin, caloric restriction and growth factor ablation, which are all well-characterized in their effects on aging of mice (11-16).

### Results

FIG. 1 shows Human-mouse epigenetic clock. DNA methylation estimates of a) relative age and b) chronological age in samples from mice and humans. The y-axis reports cross validation estimates of the DNAm based age estimator. The x-axis reports the actual values. Points are labelled by species number (9.1=mouse and 1.1=human) and colored by tissue/cell type. Analysis restricted to c) human samples and d) mouse samples. Each panel reports the Pearson correlation and the median absolute error (MAE) and median values across different tissue types. We analyzed N=1982 mouse tissues and N=1211 human tissues.

### Mouse Tissue Samples

We analyzed N=1982 mouse tissues (adipose, aorta, blood, bone marrow, whole brain, cerebellum, cerebral cortex, dermis, ear, epidermis, embryonic stem cells, fibroblasts, heart, hematopoietic stem cells, hypothalamus, induced pluripotent stem cells, keratinocytes, kidney, liver, lung, lymph nodes, macrophages from bone marrow, peritoneal macrophages, muscle, pituitary gland, placenta, skin, spleen, striatum, sub ventricular zone, tail.

### Human Tissue Samples

To build the human-mouse clock, we analyzed previously generated methylation data from n=1211 human tissue samples (adipose, blood, bone marrow, dermis, epidermis, heart, keratinocytes, fibroblasts, kidney, liver, lung, lymph node, muscle, pituitary, skin, spleen) from individuals whose ages ranged from 0 to 93. The tissue samples came from three sources. Tissue and organ samples from the National NeuroAIDS Tissue Consortium (17). Blood samples from the Cape Town Adolescent Antiretroviral Cohort study (18). Skin and other primary cells provided by Kenneth Raj (19). Ethics approval (IRB #15-001454, IRB #16-000471, IRB #18-000315, IRB #16-002028).

### Practicing the Invention of DNAm Based Biomarkers

To use the epigenetic biomarker one can typically extract DNA from cells or fluids, e.g. blood cells, whole blood, peripheral blood mononuclear cells, liver tissue, skin. Next, one needs to measure DNA methylation levels in the underlying signature of CpGs (epigenetic markers) that are being used in the mathematical algorithm. The algorithm leads to an estimate of age for each DNA sample.

### Technical Details Surrounding the DNAm Age Estimator

The human mouse clock is based on 448 CpGs. Apart from the human mouse clock, we also developed mouse clocks that only apply to mice. Some clocks apply to all tissues (pan-tissue clock) while others are tailor-made for specific tissues/organs (mouse liver, blood, cerebral cortex, fibroblasts, skin, and tails). We present clocks for liver samples. One that is a particularly accurate measure of chronological age. The other is less accurate but is particularly powerful for detecting the beneficial effect of anti aging interventions such as caloric restriction and growth hormone receptor knockout.

The mouse pan-tissue clock was trained on all available tissues. The liver and blood clock were trained using the liver and blood samples from the training set, respectively. The mouse clocks can be differentiated in terms of applicability to different tissue types: pan-tissue, liver, blood, cerebral cortex, fibroblasts, skin, and tails.

The human-mouse pan-tissue clock estimates relative age, which is the ratio of chronological age to maximum lifespan; with values between 0 and 1. This ratio allows alignment and biologically meaningful comparison between species.

### Penalized Regression Models

We developed the epigenetic clocks for mice by regressing chronological age on all CpGs that are known to map to the genome. Age was not transformed. We used all tissues for the pan-tissue clock. We restricted the analysis to blood, liver, skin tissue for the blood, liver, and skin tissue clocks, respectively. Penalized regression models were created with the R function “glmnet” (20). We investigated models produced by both “elastic net” regression (alpha=0.5). The optimal penalty parameters in all cases were determined automatically by using a 10 fold internal cross-validation (cv.glmnet) on the training set. By definition, the alpha value for the elastic net regression was set to 0.5 (midpoint between Ridge and Lasso type regression) and was not optimized for model performance. We performed a cross-validation scheme for arriving at unbiased (or at least less biased) estimates of the accuracy of the different DNAm based age estimators. One type consisted of leaving out a single sample (LOOCV) from the regression, predicting an age for that sample, and iterating over all samples.

### Relative Age Estimation

To introduce biological meaning into age estimates of mice and humans that have very different lifespan; as well as to overcome the inevitable skewing due to unequal distribution of data points from mice and humans across age range, relative age estimation was made using the formula: Relative age=Age/maxLifespan where the maximum lifespan for mice and humans were set to 4 years and 122.5 years, respectively. The gestation time for mice and humans was set to 19/365 years and 280/365 years, respectively.

### Statistical Methods Used for Building the Clocks

An elastic net regression model (implemented in the glmnet R function) was used to regress a transformed version of age on the beta values in the training data. The glmnet function requires the user to specify two parameters (alpha and beta). Since I used an elastic net predictor, alpha was set to 0.5. But the lambda value of was chosen by applying a 10 fold cross validation to the training data (via the R function cv.glmnet).

The clocks were used by employing a single elastic net regression model analysis (R function glmnet). We chose the following parameters for the glmnet R function (Alpha: 0.5, CV Fold: 10, Lambda choice for Clock based on the minimum cross validation estimate of the mean square error).

### Covariates and Coefficient Values of the Mouse Clocks

The human mouse clock for relative age is based on 448 CpGs whose coefficient values are specified in the column “Coef.HumanMouseClock”

The mouse pan tissue clock is based on 393 CpGs whose coefficient values are specified in the column “Coef.MouscPanTissue”.

The mouse blood clock is based on 112 CpGs whose coefficient values are specified in the column “Coef.MouseBlood”.

The mouse liver clock is based on 201 CpGs whose coefficient values are specified in the column “CoefMouseLiver”.

The mouse cerebral cortex clock is based on 104 CpGs whose coefficient values are specified in the column “Coef.MouseCortex”.

The mouse fibroblast clock is based on 75 CpGs whose coefficient values are specified in the column “Coef.MouseFibroblast”.

The mouse skin clock is based on 96 CpGs whose coefficient values are specified in the column “Coef.MouseSkin”.

The mouse tail clock is based on 93 CpGs whose coefficient values are specified in the column “Coef.MouseSkin”.

The mouse liver clock for interventional studies is based on 106 CpGs whose coefficient values are specified in the column “Coef.MouseLiverInterventions”.

### Age Transformation.

The elastic net regression results in a linear regression model whose coefficients b0, b1, . . . , relate to transformed age as follows

$$F ( chronological age ) = b 0 + b 1 CpG 1 + … + bpCpGp$$

Note that the intercept term is denoted by b0.

Based, on the coefficient values from the regression model. DNAmAge is estimated as follows

$$DNAmAge = F \hat{} ( - 1 ) ( b 0 + b 1 CpG 1 + … + bpCpGp )$$

where F\hat{ }(−1) (y) denotes the mathematical inverse of the function F(·). Thus, the regression model can be used to predict to transformed age value by simply plugging the beta values of the selected CpGs into the formula.

We used two types of age transformations: one for the human mouse clock, another for the remaining pure mouse clocks.

### Age Transformation for the Human-Mouse Clock

Our measure of relative age leverages gestation time (GestationT expressed in units of years) and maximum lifespan (in years). We define relative age (RelativeAge) and apply Log-log transformation as the following:

$$RelativeAge = \frac{Age + GestationT}{MaxAge + GestationT} \qquad ( 1 )$$

$$LoglogAge = - log ( - log ( RealtiveAge ) ) \qquad ( 2 )$$

By definition, RelativeAge is between 0 to 1 and LoglogAge is positively correlated with age. The coefficient values of the Human Mouse clock allow one to predicts loglogAge. To arrive at an age estimate (in units of years) one needs to apply an inverse transformation:

$$DNAmAge = exp ( - exp ( - LoglogAge ) ) * ( MaxAge + GestationT ) - GestationT \qquad ( 3 )$$

The maximum lifespan for mice and humans were set to 4 years and 122.5 years, respectively. The gestation time for mice and humans was set to 19/365 years and 280/365 years, respectively. These values should be interpreted as mathematical parameters of the formula.

### Age Transformation for the Pure Mouse Clocks

$$\underline{F} ( Age ) = \{ \begin{matrix}\frac{Age}{\text{?} + \text{?}} + log ( 1.2 + 0.06 ) - \frac{\text{?}}{\text{?}} , Age > 1.2 \\ log ( Age + 0.06 ) , Age \leq 1.2\end{matrix}$$

$$\text{?} \text{indicates text missing or illegible when filed}$$

In R software code the transformation and its inverse are given by

```
F= function(x,offset=0.06,adult.age=1.2) {
  y=ifelse(x<=adult.age, log(x+offset),
x/(adult.age+offset) +log(adult.age+offset)−
adult.age/(adult.age+offset) );
  y }
F.inverse= function(x,offset=0.06,adult.age=1.2) {
  ifelse(x<=log(adult.age+offset), exp(x)−offset,
(adult.age +offset)*x− log(adult.age+offset)*(
adult.age+offset) +adult.age) }
```

The DNAm Age estimate is estimated in two steps.

First, one forms a weighted linear combination of the CpGs whose details can be found herein.

The table reports the probe identifier (cg number) used in the custom Infinium array (HorvathMammalMethylChip40) and the corresponding genome coordinates in the mouse. The weights used in this linear combination are specified in the respective column entitled “Coef.”.

The formula assumes that the DNA methylation data measure “beta” values but the formula could be adapted to other ways of generating DNA methylation data.

Second, the weighted average of the CpGs is transformed using a monotonically increasing function so that it is in units of years.

$$DNAmAge = F \hat{} ( - 1 ) ( WeightedAverage )$$

A novel aspect of the above-noted invention is the development of epigenetic biomarkers that apply to two species (mice and humans) at the same time. A single mathematical formulas based on the same methylation probes can be used to measure age in both species based on any tissue sample (i.e. these are pan tissue clocks). The fact these epigenetic biomarkers apply to both species greatly increases the likelihood that findings from preclinical studies in mice will actually translate to humans.

One of the human-mouse clocks measures relative age (defined as ratio of age by maximum lifespan). This clock puts both species on the same footing. The relative age of 0.5 corresponds to 61 years in humans (half of 122 years) and 1.9 years in mice (half of 3.8 years).

While there are many published clocks for mice and humans, this is the first clock that applies to both species.

Novel “biomarkers of aging”, i.e. assessments that allow one to measure age, are interesting to gerontologists (aging researchers), anti-aging researchers, pharmaceutical companies that carry out preclinical studies.

Overall, we expect that these epigenetic biomarkers will become useful biomarkers for preclinical studies using mice because they capture the physiological aging state, thus allowing efficacy of interventions to be evaluated based on real-time measures of aging, other than relying on long-term outcomes, such as morbidity and mortality.

Finally, this measure may be another component of other molecular biomarkers of aging.

In summary, the invention describes novel epigenetic biomarker of aging. Strikingly, some of these biomarkers apply to two species: mice and humans.

It is critical to distinguish molecular biomarkers such as DNAm Age from clinical biomarkers of aging. Clinical biomarkers such as lipid levels, blood pressure, blood cell counts have a long and successful history in clinical practice. By contrast, molecular biomarkers of aging are rarely used. However, this is likely to change due to recent breakthroughs in DNA methylation based biomarkers of aging. DNA methylation (DNAm) based biomarkers of aging promise to greatly enhance biomedical research, clinical applications, and preclinical studies. They will also be more useful for preclinical studies and intervention assessment that target aging, since they are more proximal to the biological changes that characterize the aging process compared to upstream clinical read outs of health and disease status.

While these DNAm based biomarkers will probably not replace traditional biomarker assessments, they provide complementary information that adds valuable information, with preclinical applications.

### REFERENCES

1. Ameson A, Haghani A, Thompson M J, Pellegrini M, Kwon S B, Vu H, Li C Z, Lu A T, Barnes B, Hansen K D, et al: A mammalian methylation array for profiling methylation levels at conserved sequences. bioRxiv 2021:2021.2001.2007.425637.
2. Hannum G: Genome-wide methylation profiles reveal quantitative views of human aging rates. Mol Cell 2013, 49.
3. Lin Q, Weidner C I, Costa I G, Marioni R E, Ferreira M R P, Deary I J: DNA methylation levels at individual age-associated CpG sites can be indicative for life expectancy. Aging 2016, 8:394-401.
4. Horvath S: DNA methylation age of human tissues and cell types. Genome Biol 2013, 14.
5. Horvath S. Oshima J, Martin G M, Lu A T, Quach A. Cohen H, Felton S, Matsuyama M, Lowe D, Kabacik S, et al: Epigenetic clock for skin and blood cells applied to Hutchinson Gilford Progeria Syndrome and ex vivo studies. Aging (Albany NY) 2018, 10:1758-1775.
6. The Bc, Bock C, Halbritter F, Carmona F J, Tierling S, Datlinger P, Assenov Y, Berdasco M, Bergmann A K, Booher K, et al: Quantitative companson of DNA methylation assays for biomarker development and clinical applications. Nature Biotechnology 2016, 34:726.
7. Jylhava J, Pedersen N L, Hagg S: Biological Age Predictors. EBioMedicine 2017, 21:29-36.
8. Horvath S, Raj K: DNA methylation-based biomarkers and the epigenetic clock theory of ageing. Nat Rev Genet 2018, 19:371-384.
9. Horvath S, Raj K: DNA methylation-based biomarkers and the epigenetic clock theory of ageing. Nat Rev Genet 2018.
10. Fahy G M, Brooke R T, Watson J P, Good Z, Vasanawala S S, Maecker H, Leipold M D, Lin D T S, Kobor M S, Horvath S: Reversal of epigenetic aging and immunosenescent trends in humans. Aging Cell 2019, 18:e13028.
11. Petkovich D A, Podolskiy D I, Lobanov A V, Lee S G, Miller R A, Gladyshev V N: Using DNA Methylation Profiling to Evaluate Biological Age and Longevity Interventions. Cell Metab 2017, 25:954-960 e956.
12. Cole J J, Robertson N A, Rather M I, Thomson J P, McBryan T, Sproul D, Wang T, Brock C. Clark W, Ideker T, et al: Diverse interventions that extend mouse lifespan suppress shared age-associated epigenetic changes at critical gene regulatory regions. Genome Biol 2017, 18:58.
13. Wang T, Tsui B, Kreisberg J F, Robertson N A, Gross A M, Yu M K, Carter H, Brown-Borg H M, Adams P D, Ideker T: Epigenetic aging signatures in mice livers are slowed by dwarfism, calorie restriction and rapamycin treatment. Genome Biol 2017, 18:57.
14. Stubbs T M, Bonder M J, Stark A K, Krueger F, von Meyenn F. Stegle O, Reik W: Multi-tissue DNA methylation age predictor in mouse. Genome Biol 2017, 18:68.
15. Thompson M J, Chwialkowska K, Rubbi L, Lusis A J, Davis R C, Srivastava A, Korstanje R, Churchill G A, Horvath S. Pellegrini M: A multi-tissue full lifespan epigenetic clock for mice. Aging (Albany NY) 2018, 10:2832-2854.
16. Meer M V, Podolskiy D I. Tyshkovskiy A, Gladyshev V N: A whole lifespan mouse multi-tissue DNA methylation clock. eLife 2018, 7:e40675.
17. Morgello S, Gelman B. Kozlowski P, Vinters H, Masliah E, Cornford M. Cavert W, Marra C, Grant 1, Singer E: The National NeuroAIDS Tissue Consortium: a new paradigm in brain banking with an emphasis on infectious disease. Neuropathol Appl Neurobiol 2001, 27:326-335.
18. Horvath S, Stein D J. Phillips N, Heany S J, Kobor M S, Lin D T S, Myer L, Zar H J, Levine A J, Hoare J: Perinatally acquired HIV infection accelerates cpigenetic aging in South African adolescents. AIDS (London, England) 2018, 32:1465-1474.
19. Kabacik S, Horvath S, Cohen H, Raj K: Epigenetic ageing is distinct from senescence-mediated ageing and is not prevented by telomerase expression. Aging (Albany NY) 2018. 10:2800-2815.
20. Friedman J. Hastie T, Tibshirani R: Regularization Paths for Generalized Linear Models via Coordinate Descent. Journal of Statistical Software 2010, 33:1-22.
21. Bocklandt S. Lin W, Sehl M E, Sanchez F J, Sinsheimer J S, Horvath S, Vilain E: Epigenetic predictor of age. PLoS One 2011, 6.
22. Weidner C I: Aging of blood can be tracked by DNA methylation changes at just three CpG sites. Genome Biol 2014, 15.
23. Horvath S: DNA methylation age of human tissues and cell types. Genome Biol 2013, 14:R115.
24. Hannum G, Guinney J, Zhao L, Zhang L, Hughes G, Sadda S. Klotzle B, Bibikova M. Fan J B, Gao Y, et al: Genome-wide methylation profiles reveal quantitative views of human aging rates. Mol Cell 2013, 49:359-367.
25. Marioni R, Shah S, McRae A, Chen B, Colicino E. Harris S, Gibson J, Henders A, Redmond P, Cox S, et al: DNA methylation age of blood predicts all-cause mortality in later life. Genome Biol 2015, 16:25.
26. Christiansen L, Lenart A. Tan Q, Vaupel J W. Aviv A, McGue M, Christensen K: DNA methylation age is associated with mortality in a longitudinal Danish twin study. Aging Cell 2015.
27. Perna L, Zhang Y, Mons U, Holleczek B, Saum K U, Brenner H: Epigenetic age acceleration predicts cancer, cardiovascular, and all-cause mortality in a German case cohort. Clinical Epigenetics 2016, 8:1-7.

All publications mentioned herein are incorporated herein by reference to disclose and describe the methods and/or materials in connection with which the publications are cited (e.g. review one or more of:
www.biorxiv.org/conLent/10.1101/2021.03.30.437604v1.full;
www.biorxiv.org/content/10.1101/2020.09.06.284877v1.full;
www.bioriv.org/content10.1101/2020.05.07.082917v1;
www.biorxiv.org/coitent/10.1101/2021.03.11.435032v1;
www.biorxiv.org/content/10.1101/2021.01.18.426733v1 full;
Lu et al., Aging (Albany NY). 2019 Jan. 21; 11(2):303-327. doi: 10.18632/aging 101684; Eric Vilain, Stefan Horvath, Sven Bocklandt and UCLA colleagues: U.S. patent application Ser. No. 14/119,145, tided “METHOD TO ESTIMATE AGE OF INDIVIDUAL BASED ON EPIGENETIC MARKERS IN BIOLOGICAL SAMPLE”; “METHOD TO ESTIMATE THE AGE OF TISSUES AND CELL TYPES BASED ON EPIGENETIC MARKERS”. Inventor: Stefan Horvath. UCLA Case #2012-364 U.S. Patent Publication 20150259742, U.S. patent application Ser. No. 15/025,185; Hannum et al. “Genome-Wide Methylation Profiles Reveal Quantitative Views Of Human Aging Rates.” Molecular Cell. 2013; 49(2):359-367 and patent application publication US20150259742). Publications cited herein are cited for their disclosure prior to the filing date of the present application. Nothing here is to be construed as an admission that the inventors are not entitled to antedate the publications by virtue of an earlier priority date or prior date of invention. Further, the actual publication dates may be different from those shown and require independent verification.

TABLE 1
POLYNUCLEOTIDES HAVING CpG METHYLATION SITES

| CpG No. | CpG ID | CpG Source Sequence | Clock Species | | | | | |
|---|---|---|---|---|---|---|---|---|
| 1 | cg12841266 | CGCCTTCAAGGCGGCCGCCTTCTTCGTGCTGCTCTCCATGGTGCTGATCC | Dog | Cat | Mouse | Rat | Human | Universal |
| 2 | cg27201382 | CGCTGAAGTTGGCTTCCTAGCGGTGTAGGCTGGAATAGACTCTTGGCAAG | Dog | Cat | Mouse | Rat | Human | Universal |
| 3 | cg01712842 | CGGATAAATACACTTAAACGAGGATTTAGTTTTACTCCTGTTGAGTTCTA | Dog | Cat | Mouse | Rat | Human | Universal |
| 4 | cg23985931 | CGCCCTTTGGTGATATAAAATGCAATATTTTCAGGCAGCTATTGAGCTTT | Dog | Cat | Mouse | Rat | Human | Universal |
| 5 | cg09474015 | AGGTTTAATGCCATAAGGCCGGCTGGAGGGCAAGCCCGCGAAGGAGAGCG | Dog | Cat | Mouse | Rat | Human | Universal |
| 6 | cg05322294 | CGCCCCTTGGAGTTCTCCAGTTCTGTGAATGGTGTGCACCGTTTTCCGCC | Dog | Cat | Mouse | Rat | Human | Universal |
| 7 | cg12373771 | AGCACCAGTACAGGTCGGTGACGGCGATGAGGTACAGGTCCAGCAGGCCG | Dog | Cat | Mouse | Rat | Human | Universal |
| 8 | cg14738500 | GCTGAAAATACCTTGACACCCAATCCGAGAGGTTTGCTCATTTCCCTTCG | Dog | Cat | Mouse | Rat | Human | Universal |
| 9 | cg09227056 | AGTCAGCTAAATGAAACATTATTCTAAACATATGCATCGTAATCAGTTCG | Dog | Cat | Mouse | Rat | Human | Universal |
| 10 | cg14056866 | CGCGGCAGGACTCGAACCTGCAATCTTCTGATCCGAAGTCAGACGCCTTA | Dog | Cat | Mouse | Rat | Human | Universal |
| 11 | cg15253243 | GAGTGACGTCAGGAGCAGAGGCCGGAGCTGTCCATCAGCACCAAAGGCCG | Dog | Cat | Mouse | Rat | Human | Universal |
| 12 | cg18223258 | CGCGACCTCACAAGTCAAGCGCCTTGTTTGGATTAGCCCTCCAGATCTGC | Dog | Cat | Mouse | Rat | Human | Universal |
| 13 | cg19711579 | GCGACAGATGGGCCACTTTCTTCTGGCCACAAAGGGGCCGGAATGGAGCG | Dog | Cat | Mouse | Rat | Human | Universal |
| 14 | cg22358580 | CGGCCACTCATAAATTTGGCTTGATGACCTCCGGGAGGTTAGTGATGGAA | Dog | Cat | Mouse | Rat | Human | |
| 15 | cg07032889 | TGCACGGGCTACGTCAGAATTTACGGATCCTGACTCCAGGAAGGCATGCG | Dog | Cat | Mouse | Rat | Human | |
| 16 | cg11015311 | TGCATCATTCCTGTTCTGTTACGGGAAACAGGCAATGTCTGAGACTTTCG | Dog | Cat | Mouse | Rat | Human | |
| 17 | cg13366440 | CGGGCAGCTACTTCCCCTACAAGCACAGCTACATGTGGAGCCTCAGGTGG | Dog | Cat | Mouse | Rat | Human | |
| 18 | cg15578619 | CGCCCTGGGGAAAAGTGACTCCTCATACATATTACTTTAGCCTAATGGCC | Dog | Cat | Mouse | Rat | Human | |
| 19 | cg24712893 | GAATTGTGTGAATTATCCCAGCTCAATTCTTTAAGTTAGTCAGAGGTACG | Dog | Cat | Mouse | Rat | Human | |
| 20 | cg18753125 | TATTTATGCAAAAGTGAGCCGACTTTGTTCGCCAGGTAACCGGGCACGCG | Dog | Cat | Mouse | Rat | Human | |
| 21 | cg19365356 | CGGAGGCACAGTCAGTATGGTGCTGTGTGCTTGTTGTTTTGTTTTGGTTT | Dog | Cat | Mouse | Rat | Human | |
| 22 | cg20913114 | ACACTGCTCCTGAGAACTGTCGGAAACAATGTCTGTGGTCGGAGGTGACG | Dog | Cat | Mouse | Rat | Human | |
| 23 | cg22736354 | CTCGAGTGCAAGGTGTGCTTTGAGAAGTTTGGCCACCGGCAGCAGCGGCG | Dog | Cat | Mouse | Rat | Human | |
| 24 | cg23223379 | CGCACAATTTTATTGATTGAAGAGATTAGGACAAAAACATTAAACCAAAT | Dog | Cat | Mouse | Rat | Human | |
| 25 | cg25361778 | CATCACAAGAAATGTATGCACTGGAAACTTACAAGCTTGCTCAAGATGCG | Dog | Cat | Mouse | Rat | Human | |
| 25 | cg27386817 | AAAATGAAAAATTTTGTGTTGTTGCGTTCCCTCTCACTCACTCCCCCTCG | Dog | Cat | Mouse | Rat | Human | |
| 27 | cg11084334 | CGCCATCATCAACGTGGTGGTCTTCATCCAGCCCTACTGGGTGGGCGACA | Dog | Cat | Mouse | | Human | Universal |
| 28 | cg19719141 | GGCCACGCTTTATTAAATTCTCATAAACCTGTCAGGGGGGACAGGGCTCG | Dog | Cat | Mouse | | Human | Universal |
| 29 | cg12879445 | AACTGGACAGCACCATGTCCACCAAAGCGGAGCAGTGTAAGTAGCAGCCG | Dog | Cat | Mouse | | Human | Universal |
| 30 | cg02034779 | CGCAAGAGGCATTTTTGCAAATACCATGCAAAACAGGCAGCTGGTGTGCC | Dog | Cat | Mouse | | Human | Universal |
| 31 | cg24664689 | GCAGCCCCAGTAGCACGAGGAGGTGTTGCAGCAGTGGCAGATGTGCATCG | Dog | Cat | Mouse | | Human | |
| 32 | cg23893313 | TTGGTCATGTTCTGCAGCAGCGACTGCAGCTCCTGCGTGTTGCCCTTGCG | Dog | Cat | Mouse | | Human | |
| 33 | cg22669570 | GAATATCCCCAGTGTTCTCATCAATAATGAAAATGGAACTTGCCCCTTCG | Dog | Cat | Mouse | | Human | |
| 34 | cg05970544 | CGCCTACTTGTCTTAATTGATCCACGCCTTATGGTCTGAGCTTTCAGTTT | Dog | Cat | Mouse | | Human | |
| 35 | cg08122757 | CTTGAGGACAAACGTAACCAAGACGACCAATTGTTATTGCTAAGGTAACG | Dog | Cat | Mouse | | Human | |
| 36 | cg12015981 | TAACTGTATAATTTATAATAATTGATTTCATTTTGACTGACCACCAGTCG | Dog | Cat | Mouse | | Human | |
| 37 | cg12540775 | CGCAAAAACTATTTTAAGACTTTCTGAACTTTGACCAGATGTTGACACTT | Dog | Cat | Mouse | | Human | |
| 38 | cg09461098 | CAGCCAGTCCTCCAGGTTCAATAGGAGATGGCTGGCCACGTGCCAAATCG | Dog | Cat | | Rat | Human | Universal |
| 39 | cg17986111 | CGTGGCTCTATCTGGATCACAATCACATCTGCTCCGTGCAGGGGGACGCC | Dog | Cat | | Rat | Human | Universal |
| 40 | cg15726426 | GAGGAGATCCGCTTCTACCAGCTGGGCGAGGAGGCCATGGAGAAGTTCCG | Dog | Cat | | Rat | Human | Universal |
| 41 | cg07900685 | CGTCTGTGGCATGGTTTGCATTGGGCCCAATGTGCTTATGGTACATAAAA | Dog | Cat | | Rat | Human | Universal |
| 42 | cg10233638 | CAATCTGCTCTGAATATATATTACAGCTTTAACATCACAGAGGGAAATCG | Dog | Cat | | Rat | Human | Universal |
| 43 | cg14322760 | ATAACATTGGATTTGCCACTGCCATTTGGCCCGATAATACAGGAAAAGCG | Dog | Cat | | Rat | Human | Universal |
| 44 | cg20049544 | CGCAGAATTTATACCATCATGTCAGCAGAGGAGGTAGCCAATGGGAAAAA | Dog | Cat | | Rat | Human | Universal |
| 45 | cg24488281 | TTCGCCCCTTGGAGTTCTCCAGTTCTGTGAATGGTGTGCACCGTTTTCCG | Dog | Cat | | Rat | Human | Universal |
| 46 | cg27300575 | GAGCCTACCTGACATTTTGGAGAATTTGCTGTGCTGGGATTGATATTCCG | Dog | Cat | | Rat | Human | Universal |
| 47 | cg17458251 | TCCTTCTTCCCCGTAAAACTTTGTCCAAGTACCTCATGTACGGTAATCCG | Dog | Cat | | Rat | Human | Universal |
| 48 | cg13848598 | GCAGGTACACGAAGGCCATGATGCACAGGGGCACGTAGAAGGAGACTACG | Dog | Cat | | Rat | Human | |
| 49 | cg18473521.1 | TTACCCATTCTCGCTCGTAAATCCAGTTCAATTGTGCTAACCCAGAGTCG | Dog | Cat | | Rat | Human | |
| 50 | cg14401402 | CGGTGTGTTTTGATTATTTTTTACATGTGCCTACTGACTTCCGACATGAG | Dog | Cat | | Rat | Human | |
| 51 | cg24644551 | AACGGCTCAATAAAAGTATTCAGAATCCCTCTCAAGTTGCCTTTTAACCG | Dog | Cat | | Rat | Human | |
| 52 | cg01360767 | AATTTGTTTGCTGCCTTTTCAATTTGCCATTCAGTTTCCATGGTTTCACG | Dog | Cat | | Rat | Human | |
| 53 | cg03120509 | AATTTTCTCTTCACCTCATATTATTAAAAGTAATAAGTTTTTCTGGACCG | Dog | Cat | | Rat | Human | |
| 54 | cg04295410 | CGGGCAATTTAATTACTGTTGGTAAAATAAGAAATGAATGATTGCATATC | Dog | Cat | | Rat | Human | |
| 55 | cg04426657 | CGGATTCTCCATTTGCTTTTATATTGGAAAAATATGTGCTGTGCCAATGA | Dog | Cat | | Rat | Human | |
| 56 | cg04512256 | CGGCAGAAAATTGGCAAGGCTTAATAAACCTCATGCCTGACGCACTCCTG | Dog | Cat | | Rat | Human | |
| 57 | cg04573984 | CGCTCCATTTTATGGAAACAAGCTCTGCGGAGAGGGCTTTGATGCTCTAA | Dog | Cat | | Rat | Human | |
| 58 | cg05081470 | CGCCCAGCGAGGACATCTGGAAGAAATTCGAGCTGGTGCCATCGCCCCCC | Dog | Cat | | Rat | Human | |
| 59 | cg07803121 | CGGTCCCCAGCCCCCTGCTCAGCTCAGCCTCGGCCAGGAACGCCATGTTA | Dog | Cat | | Rat | Human | |
| 60 | cg08215831 | CGCTCTCTTGACAGCTCGATTGCGTGCTGCCTCTGCTCCTGCATAAATCA | Dog | Cat | | Rat | Human | |
| 61 | cg08707435 | GGTGGTAGTCGTCGTATCTGGGGTGGTAAAGAAGCCCAAGGTCACACGCG | Dog | Cat | | Rat | Human | |
| 62 | cg09410314 | TGCTGGCTCTGAAGCCGTGTAGAATTTCGTAATGGAATGTGAACTGCTCG | Dog | Cat | | Rat | Human | |
| 63 | cg10172301 | CGGCACCATGACCCTGGGCAGCTGCTGCTGCGAGATCATGTCCTCCGAGA | Dog | Cat | | Rat | Human | |
| 64 | cg10323846 | CTGTGACCGCTGGCTGGATGGGGTAGGCCTGCTGCACAAAAGGCTTGACG | Dog | Cat | | Rat | Human | |
| 65 | cg13222741 | CGGCTCCCCGTGACCTTATTCAATCTTGGGGCTGATCGGGAAATATATTG | Dog | Cat | | Rat | Human | |
| 66 | cg13669200 | CAGGCAGCTGACCCTGAATGAGATCTATAACTGGTTCACCAGGATGTTCG | Dog | Cat | | Rat | Human | |
| 67 | cg14403768 | CGCGGATGTGGTTAACCAGTTTGTATTTGGCTTTGAAGGGCTTGCCCTCG | Dog | Cat | | Rat | Human | |
| 68 | cg16643400 | CGGCTTCCGAATGGTAAACTGAAATGTGACGTCTGTGGCATGGTTTGCAT | Dog | Cat | | Rat | Human | |
| 69 | cg17213363 | TCTTTACCAGAATTTTCAGCCAGCCGTAACTTTCCACAACAAAGATACCG | Dog | Cat | | Rat | Human | |
| 70 | cg21249517 | GCTTCTTGCCCAGGGAGCCGCTGCAGTTGTACTGCTTGCGCAGGTTCTCG | Dog | Cat | | Rat | Human | |
| 71 | cg22875689 | CGCACCAACCAAAGACAACATATTGGGAAGAAAAATGCTTGTAAACTGCA | Dog | Cat | | Rat | Human | |
| 72 | cg23930466 | CGCAGGAGCCTTCACGGAGCAGATTCTGAGGGACATGGCCTCCTTCCACG | Dog | Cat | | Rat | Human | |
| 73 | cg25291023 | CGTCGAGAAGTATAGACCTGCCTCCGCCTCAGTATCGGCTCTGATTGGAG | Dog | Cat | | Rat | Human | |
| 74 | cg25945416 | CGCTGAGAGAAGACATAATCAATTCCCCAGCTGGCACTTTGCGCTGAATA | Dog | Cat | | Rat | Human | |
| 75 | cg25767194 | CGCGTTAACTCGGCTGGAACGCTGGTTAATTTAACAGCAGGAGTTGAATT | Dog | Cat | | Rat | Human | |
| 76 | cg26677826 | AAGGTGACAGCCAAGCCGCGTTAGCACCTGCATAAAATATTAAAGGGACG | Dog | Cat | | Rat | Human | |
| 77 | cg25947654 | CGCTCAGTTCAGCTTTAATTATGACTCTGCAGACCTGAAGAAATATTGCA | Dog | Cat | | Rat | Human | |
| 78 | cg04267424 | CCCGGCGCTTAAATCTGTGGCTTAATCCTTTTGCAAATATGCATTGATCG | Dog | Cat | | Rat | Human | |
| 79 | cg09915949 | TTCTAATGTTCCATTGTGAGGAGCTTCCATTGTGACGTCGCGCCCCTTCG | Dog | Cat | | Rat | Human | |
| 80 | cg24470719 | GATCTGTTCATCTCCGCTCTTCCTCGACTCTTCTCTCTTCTTGTCTTACG | Dog | Cat | | Rat | Human | |
| 81 | cg07175149 | CGCCCCCTACCTGGGTGCAACATACTTTGGAAATAGAAGATGACCAGGAT | Dog | Cat | | Rat | Human | |
| 82 | cg26512254 | AAGACCCCAGTGGCGCTGTTTTAAAAAGCCCCCAAGAAGTGAAGAGCGCG | Dog | Cat | | | Human | Universal |
| 83 | cg01153166 | CGATGGCTTTCCTGAATGGCTGACTGTGGGCTGCCCTGGACTTGGCCCCC | Dog | Cat | | | Human | Universal |
| 84 | cg10250869 | GGGCTCAGAAGCCAGTTGTTGGCGCTGTCCGTGGTGCTGAGGAATTCCCG | Dog | Cat | | | Human | |
| 85 | cg27487607 | CGCCGGATGCTAATGATGAAATCAAAATGTCATCCAAGTTAAATGCGGGG | Dog | Cat | | | Human | |
| 85 | cg21908378 | ACTGTACAGCCCATAAAACTCCAAACAGCCTCCAGACACTGCCGCAATCG | Dog | Cat | | | Human | |
| 87 | cg06930747 | CGAGCATATTTTGTAGCTTCTCTTCGCCAGTGGGAAGCCACCAGCAAACC | Dog | Cat | | | Human | |
| 88 | cg03638659 | CGGGCAGTGGGGCCAGTGTCTTTATCGGCCAGATGGTGTGAATTTAGACA | Dog | Cat | | | Human | |
| 89 | cg08327269 | CGCGGAGCGCTCCATTCCGGCCCCTTTGTGGCCAGAAGAAAGTGGCCCAT | Dog | Cat | | | Human | |
| 90 | cg22929401 | AGTCACCAGGGTGGAATATTTTAGCACCTATAGAAAAACCGCCACAGACG | Dog | Cat | | | Human | |
| 91 | cg00563016 | CTCTCCCCGGGCCGTGGAAAGGATCCCACTTCCGGTGGGGTGTCATGGCG | Dog | Cat | | | Human | |
| 92 | cg00674815 | TGGGGGCTGGGGTCCCTGGTCACTATGGGGAGAGTGCAGCAGGACCTGCG | Dog | Cat | | | Human | |
| 93 | cg00703273 | CGGCCTGTGTGAGCAAAGGGAAGGAAATGACACAGGCAGGAAAAAAAAGC | Dog | Cat | | | Human | |
| 94 | cg01127608 | ACTTCCGGGATCGGAAACTGTACTGCAAACAAGACTACCAACAGTAAGCG | Dog | Cat | | | Human | |
| 95 | cg01374277 | CGCAGCAAGTCAGATATGGCTTTTTTATATAATGCTAATCAAGTTTCCAA | Dog | Cat | | | Human | |
| 96 | cg03727787 | CGCTCGCATTTAAGGCATAATTGATTTTTATTGAAAAGCCAGCAGGCACA | Dog | Cat | | | Human | |
| 97 | cg04539724 | CGAGGACCACCTGGCAGTTCATAAACACAAGCATGAGATGACATTGAAAT | Dog | Cat | | | Human | |
| 98 | cg04984522 | CGGCACCAAACAGCTGTAAATTGAAGGTTGTGCTTAGTTTTCTCAACTCT | Dog | Cat | | | Human | |
| 99 | cg05613158 | CGCACCGCACTCCATATCGAGGATGGATTGTTTTATGCTGATGCAATGTG | Dog | Cat | | | Human | |
| 100 | cg05730460 | CGTTGCCGTCTTGAAGATTCAGTAGAACTACATCCCAGGCATGTGTAGGT | Dog | Cat | | | Human | |
| 101 | cg06904135 | CGCTCTCCTCCTGCAGCCGCAGCTCCTCGCCCTGCAGCTGCAGCCGCACC | Dog | Cat | | | Human | |
| 102 | cg07797033 | GCCATGATGATGTTCTCTGTGGCCATGAAAGGCAGCTCTTGCCGAATGCG | Dog | Cat | | | Human | |
| 103 | cg08437267 | CGCACCGCTTCCCCAATTAGGGCTGACAGGCTGGGGGCTACTTGGCTCAT | Dog | Cat | | | Human | |
| 104 | cg08868548 | CGCTGCCCTGTACATTTACAGAAAAGTACCTCTGATATAATAAAGGCTCA | Dog | Cat | | | Human | |
| 105 | cg08885324 | CGCTGTGCTCCTGTGAACATTTCCTGCTATGCCAAGTTTGCAGAGGCCCT | Dog | Cat | | | Human | |
| 106 | cg09610373 | CGCTTTACCATCAAGTTCCAGGAGTTCAGCCCCAACTACATGGGCCTGGA | Dog | Cat | | | Human | |
| 107 | cg09689442 | AAAAGGTTTAATTTCCATTTTCCTGCATCAGCTTTATTTATAAAATGTCG | Dog | Cat | | | Human | |
| 108 | cg10735971 | AGCGAGCCATCCTGAGGTTTAAACAGACAGAAGACACACAAAGGTGATCG | Dog | Cat | | | Human | |
| 109 | cg11155697 | GTAACCATAGAGCATGAATTACCTCTTGAGGTCATCAGCGAGAATTTACG | Dog | Cat | | | Human | |
| 110 | cg11383875 | GCGTGTGTGGGGACTTCTCACTGCCACGGACCCTTGATGCATGCTCCCCG | Dog | Cat | | | Human | |
| 111 | cg11940021 | CGCTGGTAACCGCAGCCAGCCATTATGGGCAGGGGTTTCAAACATATGGC | Dog | Cat | | | Human | |
| 112 | cg12596040 | CGGGGGTTTGGTTACTTTATTTCTAAAGTTGTTTGTATTTCTAAAGTGGC | Dog | Cat | | | Human | |
| 113 | cg12644878 | CGAGCTCAGACAGCGACAGTGATAAGTCCACAGAAGACCCCCCAATGGGT | Dog | Cat | | | Human | |
| 114 | cg12695985 | CGCTCCATCTCGTGCAGGAAGCTGTAGAACTGAGGCAAGGTTAATTCTGG | Dog | Cat | | | Human | |
| 115 | cg12945499 | CGCCTGAAGTAAGCAAATGTCCGTGTAAACCAGCTGTAAATTTCATTAAG | Dog | Cat | | | Human | |
| 116 | cg13380048 | CGCACCGCCACCTACCTACCGGAGGTTGCAAAGGAGCAAGGTCATTGTTG | Dog | Cat | | | Human | |
| 117 | cg13436384 | AGCAATGCCACCTACTCCTGTCCTCGTCAGAAGAACTGTTTGATTGATCG | Dog | Cat | | | Human | |
| 118 | cg13621099 | CGCCTTGTCATTCCTGAGGTTCTCATGGGCACTGGAAGAGGAAAAGCTCA | Dog | Cat | | | Human | |
| 119 | cg13647620 | CGAGGATAATTAAGTGGTGAATAAGGGTCAATGTGGACAAATAAATCAAA | Dog | Cat | | | Human | |
| 120 | cg14104528 | CGCTGATATTGAGTCATATAGGCCATGTAGCTAGCAGCTCGGGCTGCAAT | Dog | Cat | | | Human | |
| 121 | cg14219291 | GGCGGCGTCCATTTCGGGCTACACCTTCAGTGCTGTGTGTTTCCACAGCG | Dog | Cat | | | Human | |
| 122 | cg24089067 | GAGGAGTCACTTTTCCCCAGGGCGCAGTAACTATTTTTGCTCCCCAGTCG | Dog | Cat | | | Human | |
| 123 | cg15112775 | CGGGCCCCAAGCCCTGCCCTCAGTCAGTGAAAGCAAGTGCCTCTCATGTG | Dog | Cat | | | Human | |
| 124 | cg15582951 | CGGCTGCCTGCAACCAGGATGTTCTTATGAGCTTTGAAGATTTTTCCGCT | Dog | Cat | | | Human | |
| 125 | cg15632198 | TAATTCAGACATTCAAATGCAAAACCGCACCATTTGGAGAGTGTAATGCG | Dog | Cat | | | Human | |
| 126 | cg25922845 | TATTTCTCATCTTACTTTTTCCTTCACGTCCAGGTCACTTTAAGACTTCG | Dog | Cat | | | Human | |
| 127 | cg18634499 | GTGCTCAGCAAATACACCAACCTCCTCCAGGGCTGGCAGAACAGGTAACG | Dog | Cat | | | Human | |
| 128 | cg19382919 | ATGTATATACACAAATAATTGGATTTTCCTGATCAAAGGGGGCCTTGCCG | Dog | Cat | | | Human | |
| 129 | cg19520890 | CGGAATTGGAGAATATACTGGTCGCACCATTTTAATAGCACTTGTGCCCT | Dog | Cat | | | Human | |
| 130 | cg19721103 | CCGCCCCCTAAAGACAACATTCCCGCCCTAGTGGAGGAGTACCTGGAACG | Dog | Cat | | | Human | |
| 131 | cg19848879 | CACAGATGTTGTTTGGCTGAAGAGCAGCTGAATCCCCTCTCTGGCAGCCG | Dog | Cat | | | Human | |
| 132 | cg20830232 | TTAAATTCAGATTATGCTGAAGCAAAATGGTCCTGGTATGAGAAGCAACG | Dog | Cat | | | Human | |
| 133 | cg21081239 | GGCTTTGTTTTTGTGTTCTACCAGCAGAGGATTACATTACTGATTGATCG | Dog | Cat | | | Human | |
| 134 | cg21380560 | CGCGTCTCACGCGCTTGGGAGAGAGTTTGCAAAGTCATAATTATATCTCT | Dog | Cat | | | Human | |
| 135 | cg21821706 | GTCAACCGGGCAGAGTCTTTCAGACAGCTGTGGGACGGTGCTCGGAAGCG | Dog | Cat | | | Human | |
| 136 | cg22515205 | CGTGACAAACAGGTTGGGGTTTTGATCATTAATCCTGCTAAAGCAATATT | Dog | Cat | | | Human | |
| 137 | cg22952564 | CTATTCAACAGTCTATCTCTGTCATGGTTCATTTATGTGTGAGCATGACG | Dog | Cat | | | Human | |
| 138 | cg25872155 | GGCTTTTCTTATTCAAAACAGGCCCACAATGGGCTTCACAGTGCGCCTCG | Dog | Cat | | | Human | |
| 139 | cg23810523 | GAATCAGTACAAAATAGGTGCTACCTAAACGTTCCTTCTACCTGAATTCG | Dog | Cat | | | Human | |
| 140 | cg24315421 | GTCCCTGGTTACCGGGGCAACAGAAGCAGTGATGAAAATCATGCCTTTCG | Dog | Cat | | | Human | |
| 141 | cg24570504 | CGCAGCAGCAGCCGCAGGAGCTGGCCCGGGACAGCAAAGGCCGCTTCTTT | Dog | Cat | | | Human | |
| 142 | cg25162386 | CGCGCGCCAGCTTGTCCACCTCGCGCTCCAGCTCCGACTTCTGCTTCTGC | Dog | Cat | | | Human | |
| 143 | cg26158731 | AGTGAGAAATTCAGAAGACTGAAGCCCAGGCTTACTGTCTACCTTTCACG | Dog | Cat | | | Human | |
| 144 | cg26699785 | GTGGGACCTCTTCATGTCCCCTCAGTTAATTTCAGAATTAATTTGCAACG | Dog | Cat | | | Human | |
| 145 | cg26921187 | TGCCTGGTCATGTGTCGCCTTTTTTTGTTTGCAGTGGAAACAATTTCTCG | Dog | Cat | | | Human | |
| 146 | cg26903477 | GGCTCCAGGAAGCTGCTGCTGACAATGAATGAAGGGATACGGTTTACGCG | Dog | Cat | | | Human | |
| 147 | cg27096534 | TAATCATTGAACTTGATTGCACATTCTCTTTCCCAGTCATAGTCAAATCG | Dog | Cat | | | Human | |
| 148 | cg17136263 | AGCACGAGGAGGTGTTGCAGCAGTGGCAGATGTGCATCGAGCTGCTCTCG | Dog | Cat | | | Human | |
| 149 | cg00787665 | CGGGAGATGAAAGATAGGCCAGTTTTTGAAAATGGCCTGCGTGGCACATG | Dog | Cat | | | Human | |
| 150 | cg01403803 | TTCAAGCAATTTTAATTATATATTTGTGCTACATCAGGGACTCTGGAACG | Dog | Cat | | | Human | |
| 151 | cg19766374 | TCTTTGCTATTTCAGGGAACCAATCTTCCGCCTTCAAGGCAAATTGTACG | Dog | Cat | | | Human | |
| 152 | cg22006949 | GGGGGGAGCCTGGAAACCGCCCCGTGTCCCATTTCCTCCTCTTGTTTTCG | Dog | Cat | | | Human | |
| 153 | cg01614688 | TCTGGTGCCGTGTGTGGTTTCAGGATAAGCTGCCGATGCCACCCAGAGCG | Dog | Cat | | | Human | |
| 154 | cg18442545 | CGGCTAAGCTTGCTCAAATGTATGAAGTGTCAAAAAGTTTCTTTGACATT | Dog | Cat | | | Human | |
| 155 | cg10452170 | TGATGTGCGAGGTGATGCCCACCATCAGCGAGGATGGCCGGCGGGGCTCG | Dog | Cat | | | Human | |
| 156 | cg11102842 | GAACTCGAAGTTGCTCAGTCAATACCAAGCTATTAATCTCTGTCACCTCG | Dog | Cat | | | | |
| 157 | cg05861987 | TCAGTCCCAGACTCGGCAATTTTCTAACAAGGATTAAAGTTGTTTCTCCG | Dog | | Mouse | Rat | Human | Universal |
| 158 | cg23087015 | CGGCCTCCACAAAGGTGCGCGGGGTCAGCCCAGCCCAGCCCAGCCCCAAT | Dog | | Mouse | Rat | Human | Universal |
| 159 | cg01566077 | CGCTACCTGGACGTCAGTATCTTGGGCAAGTTTGCAGTTGTGATGGCTGA | Dog | | Mouse | Rat | Human | Universal |
| 160 | cg08007722 | CGCGGCACTGGAGCAGGTACCATGTGATCCCAGGGAGCGCCTCGTGCCCC | Dog | | Mouse | Rat | Human | Universal |
| 161 | cg23108693 | CGGGAGACAGGTGTTAAACTACTTAATCACTTTAAAATTGTTTTAATTTT | Dog | | Mouse | Rat | Human | Universal |
| 162 | cg09710440 | CATCGGCGTGCTGTGGGCCATCTTCACCATCTGCTTTGCCATCGTCAACG | Dog | | Mouse | Rat | Human | Universal |
| 163 | cg22100382 | TTAAGCACATTTACATTTGGTTCTTAAAATCCAAAATAGCCCATTTCACG | Dog | | Mouse | Rat | Human | Universal |
| 164 | cg23752476 | AATAAGGAATGACTGCCGTGACTACCCCCATCTAGCTATGCCATTCACCG | Dog | | Mouse | Rat | Human | Universal |
| 165 | cg13656236 | CCAAACAGTTAGGACACATTGAAAGAGAGCGTCAATCATGTATTATTTCG | Dog | | Mouse | Rat | Human | |
| 166 | cg27456337 | GACACCAGTTGAAAACCCCTTGAATATTATGTTGAACATTGTCAAAAACG | Dog | | Mouse | Rat | Human | |
| 167 | cg00910419 | CCCATGTTTTGTGTGTCTCTGTCCATCCAGACTCCTGACGTTCAAGTTCG | Dog | | Mouse | Rat | Human | |
| 168 | cg24260138 | CACGCACTGCATTTGCTTCTGGTGCATCTGCTGCAGCTGCTGGAGCTGCG | Dog | | Mouse | Rat | Human | |
| 169 | cg23169811 | CGCAAAGGTTAAGATGAAGTTTCGCTTCTTAGGAAAATTAATGGCCAAGG | Dog | | Mouse | Rat | Human | |
| 170 | cg02604675 | TCAGAGCCCTCTAAGCGCGATTGTTTCTCCCTTTCTAATGACATTTACCG | Dog | | Mouse | Rat | Human | |
| 171 | cg05759182 | ACAGGGACAGCACCTCATCCCCTGGATGGGGGACCACAAGATCCTCATCG | Dog | | Mouse | Rat | Human | |
| 172 | cg02238028 | CGGACAATGTGCTGAGAGCAGAAATTGGATGGTTTGACTGTTCACCGTAT | Dog | | Mouse | Rat | Human | |
| 173 | cg15129763 | CGCTCGCCCTTCTGATAGGTTTTAGAAAGGTCATAATTTAGGGTATTTTG | Dog | | Mouse | Rat | Human | |
| 174 | cg09445259 | CGCTGGTATTCATCTAATGGCTTGTCAAGACAAAACTGTCCCTGTTCTTG | Dog | | Mouse | Rat | Human | |
| 175 | cg15249459 | CACTGACAGAAACATATTTCCCCAATGAGTGACCTTCCACAGCTGTAGCG | Dog | | Mouse | Rat | Human | |
| 176 | cg27005345 | TCAGGTGGTAGTTCATGGCGTCCAGCAGCAGCTTCTGGCAGACCGGGTCG | Dog | | Mouse | Rat | Human | |
| 177 | cg08040307 | ACACGAGTAGATCGGGAAGCAGCCATACTATGGCAAGTCCCTACAATACG | Dog | | Mouse | | Human | Universal |
| 178 | cg18060631 | ACTTCTGAAGGTTATTAAACACCCGGAGGTCGTTAAATGCGCTGTTAACG | Dog | | Mouse | | Human | Universal |
| 179 | cg10501210 | CGGGACTGCGGCACCTTACGGCGGGACCAAGATTTGGGTCTGCGCAGGCG | Dog | | Mouse | | Human | Universal |
| 180 | cg04204168 | CTCTGCCGAGGCTGTTGTTCTCAATCCAGCTCAGAAACTGGCTCAAACCG | Dog | | Mouse | | Human | Universal |
| 181 | cg06250511 | TGCTACATTTTTTCTAGGAACTCTGAACACATTTCATTCTTGTGAATTCG | Dog | | Mouse | | Human | Universal |
| 182 | cg05033071 | CGACTGGATGGTCCGTTACGTGCCCTACTTCAAGGATAAAGGCGACAGCA | Dog | | Mouse | | Human | Universal |
| 183 | cg08026135 | CGGGCTAGATCTTCTTGCTGCTGCTCATAATGGTGCCGCAGCAATTTCTC | Dog | | Mouse | | Human | |
| 184 | cg07517669 | TCTTGAGTTTCATTCGGCGGTTTTGAAACCAAATCTTGACCTGCCTGTCG | Dog | | Mouse | | Human | |
| 185 | cg27556193 | CGCGTTCACATACCCGGGGAGGGCAGTAGAAAGGTGATCAATCTTCATCA | Dog | | Mouse | | Human | |
| 186 | cg17341782 | GTGAACATTTATTGCATATATATTAAAAATTATCCCGGAATCGAGTGGCG | Dog | | Mouse | | Human | |
| 187 | cg13061698 | TTGGTCTGGCGCCGCTTGGAATCTGGCTGGTTGTACCCATCAGCTTTGCG | Dog | | Mouse | | Human | |
| 188 | cg20315976 | CACTGAAAGGTAAGCTCATTTTGAAGACTGGGCTTCTAGAAGCAGAGTCG | Dog | | Mouse | | Human | |
| 189 | cg05543238 | AATATAGACTCTGCCAGGATGGGAAGGTGCTTTTCAGGCCTACGTCACCG | Dog | | Mouse | | Human | |
| 190 | cg06778009 | TGGTACAGGAACACCAAGTCTATAAGGCCATTGTGTAGCACCAGGGGCCG | Dog | | Mouse | | Human | |
| 191 | cg20382695 | TGGGGGACCAGTGCCAGCACTGCCAGGGCAGGTTCAAGTAAGTGCCTTCG | Dog | | Mouse | | Human | |
| 192 | cg27205362 | CGCCTCCCCCGCAGGTCCTGCTGCACTCTCCCCATAGTGACCAGGGACCC | Dog | | Mouse | | Human | |
| 193 | cg22479660 | CGGCTAATGCATTTTCCAATGTGTGAGGCAGATTATTAATGCGCTCCAGT | Dog | | Mouse | | Human | |
| 194 | cg15396498 | ATCAAATGGCCAATCCTGTTAGCGGCTTAAAAGAGACATAATTACCTGCG | Dog | | Mouse | | Human | |
| 195 | cg25184738 | CTCGGAGACAAACCTCCTGGTGTAAAATCAACCGTCATAAAACATATTCG | Dog | | Mouse | | Human | |
| 196 | cg11060413 | CGGTCCATTTCTGGGCGCTGCAAGATCAGCAGAAACCTGAGAAAGCGCCT | Dog | Mouse | Human | | | |
| 197 | cg21387328 | CGCTGGGTGGTTCAGACTCACATGTCACACACATGTCGTCAGCAACAAGA | Dog | Mouse | Human | | | |
| 198 | cg25876596 | TGCAAACAACCGGGAATGCGATGCATTTGTGTAGCGTGTGCAAATGGGCG | Dog | Mouse | Human | | | |
| 199 | cg27168029 | CGGGAACATGATATTGCAATTAAATTCTTCCAGAGAGCTATCCAAGTTGA | Dog | Mouse | Human | | | |
| 200 | cg14687245 | CTACCTTGCCATTGTCTAGGATATACTTGTCCATGACAGGCGCAGGAGCG | Dog | Mouse | Human | | | |
| 201 | cg13030822 | GGCTGCAGGAGCGGCAACAGCAGCGGCGGCTGCTGAGGGGACAGGAATC | Dog | Mouse | Human | | | |
| 202 | cg26736877 | ATTTGCTTTCGCTTCTGAAAGAGCCGCTATTCTTTGATGATTGGGTAGCG | Dog | Mouse | Human | | | |
| 203 | cg21949697 | GAAGGGGTCCGGCCCAGTCGAGCCTGACGCTCTCACCACAGGAGCTGGCG | Dog | Mouse | Human | | | |
| 204 | cg06147822 | ATCGAGAAGCAGCTGCAGAAGGACAAGCAGGTCTACCGGGCCACGCACCG | Dog | Mouse | Human | | | |
| 205 | cg19368807 | CGCCGAGGCCCCCTCTAGGCAGCAGCCTCAGCCCCTGGTGGAGATCCAGT | Dog | Mouse | | | | Universal |
| 206 | cg15301611 | CGGCGTGATTGAAGGGTTTTTAACAATTAACATAATCAGAAAATGAGCTG | Dog | Mouse | | | | Universal |
| 207 | cg27704080 | CGGCGACAAAGGGCAATATTCACAGTTTATCTCAGGGTGATGGATCTGAA | Dog | Mouse | | | | |
| 208 | cg04796803 | CCGCTTCTAACCCCTGTAATAAATGCATTTGGAGTAATCTGGCGATCACG | Dog | Mouse | | | | |
| 209 | cg20615601 | CGTGTACAAAAGTTCTTGGTTAATTAGGGAAATAAACACTCAGTTCTTTA | Dog | | | Rat | Human | Universal |
| 210 | cg25905455 | CGGAATTTGAAATCCCAGCGAGTCGGTGCCATTTATTTTCAGAGGCTGTC | Dog | | | Rat | Human | Universal |
| 211 | cg17604804 | GGGTGGCATTTCCTTCAGTGTTAACAACAGAGGTTAACTGCACACAGACG | Dog | | | Rat | Human | Universal |
| 212 | cg26067250 | AAGAGATGACCATGACCACCATGCCAGAAAGTCTCAACAGCCCCGTGTCG | Dog | | | Rat | Human | Universal |
| 213 | cg26148010 | CGGTTGAATGTGCTGTGGAATTTGTTTGCAAACCTACGAGAGCGAAGCGA | Dog | | | Rat | Human | Universal |
| 214 | cg08965235 | GTCTCAAGGGCCAGTGTCGGGACAGTTGTCAGCAGGGCTCCAACATGACG | Dog | | | Rat | Human | Universal |
| 215 | cg12201500 | CGTCGGAATTTGCCGAGGTATTCTTGCAGTTTGGGGTCAATGTCCAAATT | Dog | | | Rat | Human | Universal |
| 216 | cg22805522 | AATTTTACTAGCTTTCTGCCCCTAACTGCTCTCTCATCGTTAATTAAACG | Dog | | | Rat | Human | Universal |
| 217 | cg14550108 | CATCATGACATGGCAAACGGCTATATAAATGAATTTTTCTAAACCACACG | Dog | | | Rat | Human | |
| 218 | cg18473521.2 | TTACCCATTCTCGCTCGTAAATCCAGTTCAATTGTGCTAACCCAGAGTCG | Dog | | | Rat | Human | |
| 219 | cg06247149 | CGCCGGAGGGAACTCTCAGACCGCTTGAATCTTAGTGACCAGCAGGTCAA | Dog | | | Rat | Human | |
| 220 | cg10508317 | AGCTGAAGGTCTTGAGGCGCAGGCTGGTGTCCAGGGGGGGGCTCATCCCG | Dog | | | Rat | Human | |
| 221 | cg12195149 | CGGCCCAACGTGCAGGACCAGCTCCAGGTAGAGAGGCTGCAGCACACATA | Dog | | | Rat | Human | |
| 222 | cg17230508 | CGCCAGGAATTGCGGGGCGGCACCTTTTAAATATATAATTGATTCTGTTT | Dog | | | Rat | Human | |
| 223 | cg14475241 | ATGAATTTCACCACCAAAAACACAACAACTTCTCCAATCATAAAATACCG | Dog | | | Rat | Human | |
| 224 | cg18588934 | CGAAAAGATCAGTATCTGTCAGTCTGACAGCTGATTGGCTGCTCGGAGAT | Dog | | | Rat | Human | |
| 225 | cg03180785 | CGCCTTTCCAGATGCAAAAGTTCAGCCCCTCTGATGTCATGACGGATGAA | Dog | | | Rat | Human | |
| 226 | cg03214473 | AGACACCTGGATAGCAGACTTGGCTCTGATTGGGCAGCTCAGTAGCAGCG | Dog | | | Rat | Human | |
| 227 | cg05361761 | GGCACACGTTGATGGCCATGTGCTCCTGGATGTTGATGGTCATGCGATCG | Dog | | | Rat | Human | |
| 228 | cg04629621 | CGCGGAATAAAAGATGCTACAGAAATCATCTTTGAGATGTCAAAAGACGA | Dog | | | Rat | Human | |
| 229 | cg06548914 | CGCCCTGATTGGGATCAGATAAAGAGGGAATTTTTACAAATTAGGGAATA | Dog | | | Rat | Human | |
| 230 | cg22972685 | GAAAGCAAAACATTCAATCAGGCGGATTAAGTGCGTAATGCGTCTCATCG | Dog | | | Rat | Human | |
| 231 | cg08711810 | CGCCCCTGAATTCGCATCCGAATATGAGGCATTTTTATGTGAAAATGGAA | Dog | | | Rat | Human | |
| 232 | cg15917885 | CGGCCTCATAAATCAGTTTTTAAGTAGCAACCACATGGACATTTCCCAGC | Dog | | | Rat | Human | |
| 233 | cg12456170 | CGTCAAGCTGAATTAAGCATACAAATCAGATGAGGCATAGCAGATTACAC | Dog | | | Rat | Human | |
| 234 | cg19826361 | GGTCAAGGGGCTTCCGAGGTCATTTTGTGGTTCTAATACTGAGCTAATCG | Dog | | | Rat | Human | |
| 235 | cg00109797 | CGGGGGGAAGAGGCTCAATTTGTAGCCTATTATCCTATACGAAAATGTCC | Dog | | | Rat | Human | |
| 236 | cg07150184 | CATGTACATGCTGCTGAAGCACCTGGTAGACAGGTACAATCTCTACTACG | Dog | | | Rat | Human | |
| 237 | cg01180566 | GCATGATTTCCTTCTGTATCTTGGGGTCCTGAGTATTAATGTTGGCATCG | Dog | | | Rat | Human | |
| 238 | cg01193554 | CGTGTCTTTAAAATGCAACTGCAGGTAGAGAAGACTGTTAACCCTTTGCT | Dog | | | Rat | Human | |
| 239 | cg01351663 | GCTTGTAACTGTTAAAAATGACTTGTTAGGAAGCGTGTGGGCTCTCATCG | Dog | | | Rat | Human | |
| 240 | cg03125191 | TCCTAGCTCTTGCTCAGAGTTGAGGCCTTGGTCGGGTATGTGTGCGTGCG | Dog | | | Rat | Human | |
| 241 | cg03948870 | CGGCACCAACAGATTTACTTGATTTGCAAGTTAACTACAACATTAACTGG | Dog | | | Rat | Human | |
| 242 | cg06857779 | CGTGCTAACTCCAGACTATATTCTCAGACGGGGATGGAGTATTTTCGAAC | Dog | | | Rat | Human | |
| 243 | cg06960698 | CGTGGCTACGTCATGGAAAGCCAGTCTTTTCAATGCAAACTTCAACTCCT | Dog | | | Rat | Human | |
| 244 | cg18528188 | GACACGGACCCTGAGCTGGTGTTCCTTTGCCGCCATGTCTATGACTTCCG | Dog | | | Rat | Human | |
| 245 | cg07421911 | CGTGAGAAGGAGCTTTGTGTTTTGGAGTCTGGACTGGAACCAAAGCAGAC | Dog | | | Rat | Human | |
| 246 | cg09237433 | GCTTCCACTGTGAACGCCTCCCGGGGAATCTCAGCCAGCACTTCTGGACG | Dog | | | Rat | Human | |
| 247 | cg10221825 | TGGGGGATTATAAATGTTCACGCAATCAAGTTCATTAGGACTTCAAATCG | Dog | | | Rat | Human | |
| 248 | cg10529428 | TATAGAGTGTATAAAACAAGTTTCCCCTTCTTATTTTCTGCTTTAAAACG | Dog | | | Rat | Human | |
| 249 | cg10820947 | CGGAGCAAATCTGGCCCAGAAGGCTGCACATATTTTTGCCTGGATAGACT | Dog | | | Rat | Human | |
| 250 | cg22235058 | CGGACCGCTGCCAAAGTCAGAGGGAAAATGAAGTCTTTGTTAAATGCCTT | Dog | | | Rat | Human | |
| 251 | cg11629501 | TTTTGGTCACTCCACAGGGCTAAAATAGCCGAGTTGGCTTTAATGTGTCG | Dog | | | Rat | Human | |
| 252 | cg12597232 | CGCCCAGGCCTGCCTCGATGGCCATCACTACTTCCAAAGACAGGTTGTAG | Dog | | | Rat | Human | |
| 253 | cg12719939 | CGGCCAATAAAAGGTTTATGACATCTGACCGGCAGTGAAGGTATGCGAGC | Dog | | | Rat | Human | |
| 254 | cg13329717 | CCGAAACGAAAAGACAATTTACCCATCACTGAGTCTCTGCTGTTTCCTCG | Dog | | | Rat | Human | |
| 255 | cg21878650.2 | CGGCTTACTTTAGCTACTTACGCTGGACTGTCACAGTGTCTTAGGGATGA | Dog | | | Rat | Human | |
| 256 | cg14838851 | CGTCCTGTGCCCAGATAAACAAGAAAGAGAAAAGGAGTCATCATGTCAAC | Dog | | | Rat | Human | |
| 257 | cg15091323 | CGGGAGAGCCGGGTTAGAAAGAAGGAGACTCCAGAGAAAATATCTTCATC | Dog | | | Rat | Human | |
| 258 | cg17077229 | CGCCGCATGGTCAAAACTCAGGCCTTCTACTGGACTGTACTCAGTTTGGT | Dog | | | Rat | Human | |
| 259 | cg18217426 | CGCAAAAGTCCGGTTGCAGAGCTTACATTGGAATGTGGAGCCCAAGTCTT | Dog | | | Rat | Human | |
| 260 | cg18599609 | TCCGTGTATGGCAGCTTGGTGGACACGTGAAACATGATCTCCTTGTTGCG | Dog | | | Rat | Human | |
| 261 | cg19269615 | CGCTCCCTGGAGAATTTCAGTTCTGTAAATGAAACAATTATTTTGGCAGT | Dog | | | Rat | Human | |
| 262 | cg26848592 | CGCTGGGTCAATATAGCTGCTTGTCAATTCAGAAATGCAAAGTGCTGTGT | Dog | | | Rat | Human | |
| 263 | cg27274433 | AAAAGTTAACACTACAGCAACGCTATATTTACCAGGGCCTGCCCATATCG | Dog | | | Rat | Human | |
| 264 | cg09387107 | CGGCGCTTCCGAGCACCGTCCCACAGCTGTCTGAAAGACTCTGCCCGGTT | Dog | | | Rat | Human | |
| 265 | cg24396988 | GAATGTAATTAGGTTGTCATCCTGGGGCCTCCGAGCCAGGCTTCCGAACG | Dog | | | Rat | Human | |
| 266 | cg18475091 | CGCCACCGGGAAAACCCAAGAGTACAATAATAGCCCCGAAGGCAGCTCCA | Dog | | | Rat | Human | |
| 267 | cg25485747 | CGTCAGCCGTCTTTCACCAACCTCAAAGGAAAGTTGAATGCGGGGCTCAC | Dog | | | Rat | Human | |
| 268 | cg25534230 | TGACCAAATCGAGCAGTTCAACGACCACATGAGGATGCATGTGTCTGACG | Dog | | | Rat | Human | |
| 269 | cg25841491 | CGCCATTCATCAGTTTATGCATTGTGCGCCAGGGCAAATAATGACAGAAT | Dog | | | Rat | Human | |
| 270 | cg21732208 | CGCACAAAGAGGATCCCATGGCAACAAACAGCGAGGCCCACAGTTGGCTC | Dog | | | Rat | Human | |
| 271 | cg13566279 | CGCCCTAACAAAGAGGCAGAAACTGTGTCTCAACAGCCACCAAATCCAGT | Dog | | | Rat | Human | |
| 272 | cg05078979 | CGGAACTCCCAGGAAACCTGGATGAAGTGTAATTATCTCAGCTCAATCAG | Dog | | | Rat | Human | |
| 273 | cg17314597 | CGACCTTAAAGAGTCTGCGAGGCTGAATGAATTGGTGCATCTCGCTGCAT | Dog | | | Rat | Human | |
| 274 | cg18288197 | GAGGAAATAATAGCCATTTCCAATGATAATACACAATTTTAGCACTGACG | Dog | | | Rat | Human | |
| 275 | cg13500074 | AAAAATGGCAAAAGCCCCCCTGAGCTGCAAGTTCAAGTGCGGACGTGACG | Dog | | | Rat | Human | |
| 276 | cg15545854 | CGAACAGAGGAGAAGCCGAGTAGGGAGCAGGTGGCTATCATGAGAACACA | Dog | | | Rat | Human | |
| 277 | cg01196619 | TTAGCCATTATAGGCTAATAAATCATACTTTCTCTTTGAGTTGTCAATCG | Dog | | | Rat | Human | |
| 278 | cg06022033 | TGGGTAAATGGAGTTGTCTCTTTAAAGTGCCCCGGTCAGTTCACCACACG | Dog | | | Rat | Human | |
| 279 | cg19719034 | CGCTTGTTATAGTCCAATAAGCCGTGCCAGTCAAACAAGAACCCACACTA | Dog | | | Rat | Human | |
| 280 | cg15958715 | CGTGGTGCGCTATGAGACCCCTGAGGGCACTTTCTGGGCCAACAACCACG | Dog | | | Rat | Human | |
| 281 | cg23976215 | CGAGGCATTGCTAGTGACCAGTTTGTAAAATGCAATCTGTTATTTTATTT | Dog | | | Rat | Human | |
| 282 | cg11552482 | CTTTGGAAGTTTATAGCACCCTCTAATACCTCTGGAGCCATGTACCTCCG | Dog | | | Rat | Human | |
| 283 | cg19917435 | CGCTGGATTGAATACTAGATTACTCCAAAGTGGCTGAGGGTCAGGGATCA | Dog | | | Rat | Human | |
| 284 | cg26112578 | TTGCGGCCGTGGTTGTTGGCCCAGAAAGTGCCCTCAGGGGTCTCATAGCG | Dog | | | Rat | Human | |
| 285 | cg07588415 | GCGTTCCCAAAATATGACCAGGGGTGCTTGGATGTGTCGGCAGTATGACG | Dog | | | Rat | Human | |
| 286 | cg19626073 | CGGATGCTCTTGCGCTGCCATTTCTGGGTGCTGTCACTGTCCTTGCTCAC | Dog | | | Rat | Human | |
| 287 | cg22789469 | CGCCAGAAGCCGAGGCACAAACAGACGATGCCCCATCCCCAGAAGTTCCA | Dog | | | Rat | Human | |
| 288 | cg18533392 | CGCACTAGCATGGCGCCAGCCATGTACTTACAATGTAGTTACAGAGTAAT | Dog | | | Rat | Human | |
| 289 | cg24505061 | TTTCTTCCTGTGCCTGAACCTCTCTTTCGCTTTTGTGGAACTACTCTACG | Dog | | | Rat | Human | |
| 290 | cg15674081 | AGTCATTATATCAATTTTTGTTACCCAGATACTTCAGTCCCAGTTAAGCG | Dog | | | Rat | Human | |
| 291 | cg16632407 | CCAAGGAGGCTTCAAGACAAAGGGCCCCTTACACACCAGACAGGTAAACG | Dog | | | Rat | Human | |
| 292 | cg15985481 | TGCTGGAGTAGCACTCCAGGTCCTCGCTGATGCTGCTGATGATGCTGACG | Dog | | | Rat | Human | |
| 293 | cg18357583 | AGCTCCCCAGAGGCCCTTAGACTGAGAACTATGATTGTGTTGGAGAAACG | Dog | | | Rat | Human | |
| 294 | cg02783173 | CGCTGAACCAGGAGACAAACACGATTACCAGCTCCGAGCCTTGAGTCAGA | Dog | | | | Human | Universal |
| 295 | cg18961151 | CGCCACCCAGCGGGCAGGGTGACGTCACCGTCTCCCTGACACTCCACATT | Dog | | | | Human | Universal |
| 296 | cg05916870 | GCTGGGAGCAGCCATCCCATAATAGCTGGCCGCCTGTTTTACTGGTTACG | Dog | | | | Human | Universal |
| 297 | cg05039938 | CGGAATTTCGTTTTCAAATAAACCACAAGCTTGGATGAGCTAATGTTGTT | Dog | | | | Human | Universal |
| 298 | cg07547549 | GCTCAGCTCCATTGGAATGCTCCGGGCGCTGTCCAAGGTGCTGGAATGCG | Dog | | | | Human | Universal |
| 299 | cg18304538 | AATATTTGTGACAGATACAACAAATTCTTGCACAAATTACATTTCATTCG | Dog | | | | Human | Universal |
| 300 | cg04414148 | ATACAAAACCGGGGAGTCCCTGCGCCCCAATTATTATGACTGCGGCTTCG | Dog | | | | Human | Universal |
| 301 | cg16286672 | CGGACCTAACTTTTAGAGGATCAATGTGACAAGGCAGTCCCTGATAGATG | Dog | | | | Human | Universal |
| 302 | cg07332354 | TGGTGAATCAATCTTTCCATTCCTGACATTTTGCTAATTTTAGTTTTACG | Dog | | | | Human | Universal |
| 303 | cg07026794 | CGCTTTACATTTCGAAGAAGGGATGCCAGTTCATAAACAGTTTTCGGTGA | Dog | | | | Human | Universal |
| 304 | cg26072749 | TATTCCCCTAGATACGAATTTGTGACCACAAAATTCCTTACACAAATTCG | Dog | | | | Human | Universal |
| 305 | cg03190580 | TTTGTCTGGTCGCCGCGTAATTCGCGCATCCTTATTCAGATTTGGTGACG | Dog | | | | Human | Universal |
| 306 | cg03513784 | TGAGCTAGGCCTATTTCCCTTTCTGTTACTATGACAACGAAATTTTACCG | Dog | | | | Human | Universal |
| 307 | cg03898602 | GTGTACAAGGGCGAGTGGACGCACGGATTCAAGGGGCGCTACGGGGTGCG | Dog | | | | Human | Universal |
| 308 | cg08587692 | CGAGTGAGGGAATTGCCATGCAGAAATAAATGGTAAAACTGGGAAGTCGG | Dog | | | | Human | Universal |
| 309 | cg10801485 | GGAGAGTTAGTGCTGTCAGTTTCTCTCACCTCAGAAAGCCGCCGCAATCG | Dog | | | | Human | Universal |
| 310 | cg11355954 | CGCTGATAGAATAATAAATGACAGAGTAATTACCATTATAAAGAGGGAAT | Dog | | | | Human | Universal |
| 311 | cg14361875 | CATATAAGGGCTTTTGATACACATGACTTACTTGGATGACAAGGGGCTCG | Dog | | | | Human | Universal |
| 312 | cg15471501 | GTTCAAATTGCCAGCTTCTTAAAAACCGCCCATTGGAATAAATTGCATCG | Dog | | | | Human | Universal |
| 313 | cg15809488 | CGTGCCAAAAAAGGGGCAGAGTTTGATGGAGTTGGGTGGACTTTTCTATG | Dog | | | | Human | Universal |
| 314 | cg16756969 | TTCAGCAAAGTTTATCTGCCCGACATAGCAGGAAGTGCTAATTTCATCCG | Dog | | | | Human | Universal |
| 315 | cg17357006 | AAGCGCAAATAAGCTCCCAATTGTCTCCCCAATATGTAAACCACATCTCG | Dog | | | | Human | Universal |
| 316 | cg17369088 | CCTCAGCCTAACTTCCTTTATTTCCTCACAAGTTTCTCCCTCAAACTTCG | Dog | | | | Human | Universal |
| 317 | cg25906744 | AGGGGCGGAGCTGTGGGAGGCAGCCATGGGTGCCATAATAGGCACCTGCG | Dog | | | | Human | Universal |
| 318 | cg21621248 | CGTGAGCAGCTTACGACACTCAGTGAGCAGTAGGTGCCTGTGCAAGCTCG | Dog | | | | Human | Universal |
| 319 | cg25383995 | GGTCCCTGTCGGTGCCACGGACGCTTAAATAAAATGAATCTGTATTTCCG | Dog | | | | Human | Universal |
| 320 | cg13753818 | ATTCTCCTCATGCTATTGTGGTAAGAGGCGCACTTGAAATCTCTCAAACG | Dog | | | | Human | Universal |
| 321 | cg21673805 | ATGATTACCTGCTGCTGCTGCCGCCGCGGCTGAACTCTCATCTTGACTCG | Dog | | | | Human | Universal |
| 322 | cg04267101 | CGGGTCTTTGCAACCTGGGGTGCCTTGACCAACGAGTCAAGGTTGACCAA | Dog | | | | Human | Universal |
| 323 | cg02909927 | TCTCTCCTTAATAATAAAATTATACCAAGCAGTTTGCGGTGTGGCAGGCG | Dog | | | | Human | Universal |
| 324 | cg07323940 | TCAGTGCCTTGTTGGCTAAAGGCCAGAGTGCCATTAGAACATGCTGTACG | Dog | | | | Human | Universal |
| 325 | cg18691948 | CGACTCTGCTTGAAGGAATGTGACAATAAAGTGGGAAACCAAAGCTTGGC | Dog | | | | Human | Universal |
| 326 | cg21452781 | CGATTCCCCACAAAGGTTTCAGCACCATGACCAATTGGTCAGCTCCTCCG | Dog | | | | Human | |
| 327 | cg11373055 | CGGGTCTGAAACTACCTGTAGCTGCAGCTACCTGCCCGCCCCACCTCGGG | Dog | | | | Human | |
| 328 | cg14540643 | CGCCCTCGAGCGGAGCAAGGCGATTGAGAAAAACCTCAAAGAGGATGGCA | Dog | | | | Human | |
| 329 | cg16448606 | CGTGCTCAGAAGTGTTGAAAGAAAGGCCCATTCTCTGCCCAGGGAATTGC | Dog | | | | Human | |
| 330 | cg17596554 | AGGAAATTTAAAGGCTTGGACCCTGCGAGACAGACAAACCGGTGCCAACG | Dog | | | | Human | |
| 331 | cg11023965 | TCCATGATGACCAGAGTTGGCTGGCCCTGAGATACCACACCCAGGAGACG | Dog | | | | Human | |
| 332 | cg11551236 | TTCCACCTAAAACGTTTAGTTTCAGCTCAGTGATCAGCTACCCCAGCTCG | Dog | | | | Human | |
| 333 | cg19828220 | GGAAATGATAAAAAGTTCATGTTCACGATCGCCCGGCCACATGACCGGCG | Dog | | | | Human | |
| 334 | cg03717442 | CGGGCAGAAAACATAAAAGAGGTGACAATTGTATACTTTCTAGGACAAGC | Dog | | | | Human | |
| 335 | cg00295657 | ACTGACCAATGGCAGAGGCAGGAATTGTCAAATAGCACCCAGGAGGAGCG | Dog | | | | Human | |
| 336 | cg04426458 | CGGCATATTGCCTTTTTCAGTGAGGGCATGGCTGTTTATAGCACTTTCAT | Dog | | | | Human | |
| 337 | cg26052854 | CGAGCTCATGCTGGCAGAATAGATTGGGGTTTTAAAACCCCAATTACTTT | Dog | | | | Human | |
| 338 | cg20692569 | ACGTGTGGCCCAGCAGGTTGGGCATGCGGGTCAGGTTGTAGCCGATGCCG | Dog | | | | Human | |
| 339 | cg00720104 | CGGGGAGGGATCATGGATACTTGTCTGCCGGCTTCTGGTTCCCACGCAAG | Dog | | | | Human | |
| 340 | cg07878252 | TGAAGTTCCTATGAAACTATTAAACCCGGGTGGAGGTCAGGCTGGAGGCG | Dog | | | | Human | |
| 341 | cg01572513 | CGCTTTGAAAGAAGTAGAACAACATCTCTAAACAAATTATGACTGGGCCA | Dog | | | | Human | |
| 342 | cg21826256 | ATCCATTGTGGCACAATGTAATATGTATCACCATGCTACACCTGCGGACG | Dog | | | | Human | |
| 343 | cg15660194 | CGATGCATAGAGTGCAAGATGATATGCCCTTCTTGGTCCAGTGTATTGTT | Dog | | | | Human | |
| 344 | cg02520894 | AGCTGCAGAAGCAGAAGTCGGAGCTGGAGCGCGAGGTGGACAAGCTGGCG | Dog | | | | Human | |
| 345 | cg04561722 | GGGCAGGATGGAGAGCGTCTTCAACCAGGCCATCAGGAACACCATCTACG | Dog | | | | Human | |
| 346 | cg19428989 | AAAGTAACAATCCGCCATTACAAAGGTCGTCCTCCTTGATCCAGTTAACG | Dog | | | | Human | |
| 347 | cg10956761 | AGTCAGCACAGCCATTCTCTTCCTGATGAGGGACTTTGAAGACAGTGACG | Dog | | | | Human | |
| 348 | cg19553651 | CGAATTCACTCAGTAAAATTAGCATAAGTATTTACCAGATTAAGCTGACA | Dog | | | | Human | |
| 349 | cg15359095 | TCAGTTTATTGGCTTGGGATCCTGTTTTTTTCACCTTCATAGGAGCATCG | Dog | | | | Human | |
| 350 | cg27564788 | CGCAGGCAGGCCTGCGTGCGGACAGATTTAATATCGTTTGTAAATATGAT | Dog | | | | Human | |
| 351 | cg13673047 | CGTCACGGAGCTTTCCCGGGGCTCAGATAAATAGGCTGGTGGAGTTCCCT | Dog | | | | Human | |
| 352 | cg26360901 | CGCCTTCATGTCGCTCAGCAACTCCTCCAGCTGGTTCAGAACTGGGGCCC | Dog | | | | Human | |
| 353 | cg21107288 | CGCCAGGACCCAGCTCATATCCTGTGTAACCCAGTGGGAGAGGAGCCACA | Dog | | | | Human | |
| 354 | cg18117260 | CGGGAAGGGCTAATTAGCCACCTTCTGGATCAATAAGATTCAGCAAAAAA | Dog | | | | Human | |
| 355 | cg09821379 | CGAGCTGCTCCACTTTCCAGCCAATGTCAGGAGGGGGGATCTCCGCCGCC | Dog | | | | Human | |
| 356 | cg21821439 | CGCCAACTATTCTGGGAGATACACGAGCCCTCCATGTTTATCAGGTTGAG | Dog | | | | Human | |
| 357 | cg13035951 | CGGCGCGAGTTGCCTCTCCGCTTCACGCTCGATTTCCAGGCATTCTTCCC | Dog | | | | Human | |
| 358 | cg23753196 | CGCGTCTAGGCTGAGATGGTTCGCGTGGATATTGCTACTCATTCTAAGAC | Dog | | | | Human | |
| 359 | cg25235127 | GGCTGGCGAAAGACTTCCTCAGGCTGCGGATGGTCTCAGCTTTCACCTCG | Dog | | | | Human | |
| 360 | cg09173346 | CGCTGTTAACGAGGTATTAATCAACACCCCTCAGGAAAAAATAAAAAGAC | Dog | | | | Human | |
| 361 | cg14484649 | GAAGGTGAACCACCGCAAGTACCTGCAGAAGCAGATCAAGCGCTGCAGCG | Dog | | | | Human | |
| 362 | cg13802429 | TATAATTATCGGAAGCAGTGCCTTCCATAATTATGACAGTTATACTGTCG | Dog | | | | Human | |
| 363 | cg16781658 | AGAAGGACCTTGTAATAAATAATATTCTGCACATCAAATCACTTTCACCG | Dog | | | | Human | |
| 364 | cg18174316 | GCCCTATTCCCTGGAGATTCTGAGATTGACCAGCTCTTCCGGATCTTTCG | Dog | | | | Human | |
| 365 | cg19104072 | GGATCACATCGTTGGGGTCCATGGTGGTCTTCAGCAGGCCCCTGTAGACG | Dog | | | | Human | |
| 366 | cg19965314 | CTCCTAGGAGCTCACAGCTCCAAACATCAATTACCATGATTATCTACCCG | Dog | | | | Human | |
| 367 | cg05866783 | GCTCACTCCGATGGGCTTGCTGACCCCAACAGGCCGGCTGATGGTGACCG | Dog | | | | Human | |
| 368 | cg07386222 | CTAGCTCTTGCTCAGAGTTGAGGCCTTGGTCGGGTATGTGTGCGTGCGCG | Dog | | | | Human | |
| 369 | cg03199779 | AGGAATGCGCTGAAGCTTGTAATCAGCTTTGTGAAGCCATTGAGTGCACG | Dog | | | | Human | |
| 370 | cg08035723 | CGCAGACAATGCCTCAGTGATAAGCATGCAGATTGTGCACCTAGTTCAGA | Dog | | | | Human | |
| 371 | cg11065059 | AAGAGATGAGGCTCACGTTGCACGGATGACATCACTAGCTTTTGGCTGCG | Dog | | | | Human | |
| 372 | cg08464821 | CGAGAAGAAGTGGCGGCACAAGTCGAGCGCCCCCAAACGCTCGATCCCCC | Dog | | | | Human | |
| 373 | cg08530978 | CGCCTCGGACCAATCCCCAGTGATTATGCAAGACAGCGGACCAATCAGCT | Dog | | | | Human | |
| 374 | cg21144428 | TTCTGTCCCAATGTGCCCATCATCCTGGTGGCCAACAAAAAAGACCTGCG | Dog | | | | Human | |
| 375 | cg25273233 | CGGCGGGACCATTAATCCTCAGCATGGGGTATTGTCCTCCAGACAGTTAC | Dog | | | | Human | |
| 376 | cg26440026 | TGTACACAGGCTAAGTGGCCCCACTCCAGGACAAGTGGCCCTCTAAGTCG | Dog | | | | Human | |
| 377 | cg09407361 | CGCTCAGGAAGCCTTCTGTTTAGAAAACTGGTGTGTGCGTTTTTTGATGA | Dog | | | | Human | |
| 378 | cg14283980 | AACAAAACATAAACGATCAATAAGTCGATGGCACTTCACATGATTAACCG | Dog | | | | Human | |
| 379 | cg18645607 | CACGAGTAAATTATTCATGATTTTACGAGCTCTTTAGCTCCATTGAATCG | Dog | | | | Human | |
| 380 | cg13385016 | CGCTTGCGAGCAAGGGAGTGTATATACTAAATGCCACACTGTATGTTTCT | Dog | | | | Human | |
| 381 | cg00179574 | CGGGTTTGCCTACAGGTAGATTAGATTAATTATTACTATGTAATGCAAGT | Dog | | | | Human | |
| 382 | cg00622300 | CGCTTTCACAGTCAATGAAACCACCTGAATAGATGAAAGCCAGACAGCTT | Dog | | | | Human | |
| 383 | cg00843538 | CATTTTCCCAGACAATGGTGACTACAAAGAGGCTTCAAAATAGAACATCG | Dog | | | | Human | |
| 384 | cg01743031 | GTCTTGAGAGACTGGATACATAAACTGGCTCCAGCCAGCTGCGGCTGTCG | Dog | | | | Human | |
| 385 | cg01988539 | CAATAGTGGGGGTGGCTGCGTGGGTCGCCATGGGGACGGGGCTGTTCCCG | Dog | | | | Human | |
| 386 | cg02037503 | CGGCAGCGATCAGCCGGAGACATGCAGGGGAGCCATCTTGCCACTTACCC | Dog | | | | Human | |
| 387 | cg02187860 | CCAAGATTTAAAAGCTCATCGCCGGCCTTGTAATGCAGTAACTGATTGCG | Dog | | | | Human | |
| 388 | cg02292529 | TTAACATGCAGCCTGCAAATCAGTAACTTTGCAGCTCAGACTCGCAGACG | Dog | | | | Human | |
| 389 | cg02803284 | TGGCTAGTGAGATCAGGTAAAACCTGATGCATGCTGCATGCTGCTGTGCG | Dog | | | | Human | |
| 390 | cg02846173 | ACAGTTTGGTCGGATCTTTGAGCTGACTGTCATCAAGGACAAGTACACCG | Dog | | | | Human | |
| 391 | cg09776887 | GCCTGTTTCTATTAAGCAGTTCCATGGCCCTCGGCGTGGGTGGTCTGCCG | Dog | | | | Human | |
| 392 | cg03225524 | CGCGAACCAGAAAGAAAAGTATGAGGCTGACCTAAAGAAGGAGATTAAGA | Dog | | | | Human | |
| 393 | cg03242400 | CGACCCGTTTGCATCCATGCATAGTAAGTAGGCGGCGAGCTCGCTTCTCT | Dog | | | | Human | |
| 394 | cg03327468 | CGCCCTTGGTCTGTTCCATTGGCGCACGTTGCCAAAGATGGTCATTTGCG | Dog | | | | Human | |
| 395 | cg03485005 | GAAAATGCCACCAGATGGGGGGTTAGGATTGCAGCTCCGTTGAAGGCGCG | Dog | | | | Human | |
| 396 | cg03617050 | CGCTACCAACAACAATTAAGGCATCAAAAGTGTTCCATGCATCAGTGAAA | Dog | | | | Human | |
| 397 | cg03717201 | CGTCAGGAGTCTGGATGGACAGAGACACACAAAACATGGGCAGGGCGAGC | Dog | | | | Human | |
| 398 | cg03938432 | CGCGTGCCTGTGCTGGTGTCTTAGAGCAAGAGCCTCCCTGAGCTTTCGGA | Dog | | | | Human | |
| 399 | cg04327947 | CGCTGACATCACCATCCGGGGACTCCGGGCCCTTCCCCAAAGTTAACCCT | Dog | | | | Human | |
| 400 | cg12632056 | CGCCCAAGGGCAATTTTAATTGTCTAACCCGGCCCGATTTCCTAAGAGGC | Dog | | | | Human | |
| 401 | cg04472379 | CGGTCCAACACAGTTGATATACATTTGTTTACCTGTTATAGTTGCAAGTT | Dog | | | | Human | |
| 402 | cg04958799 | CGGGCAAAAAGCAGGAACTTGTCAAGACCTGATCAAAGGCCGAAAGGGGT | Dog | | | | Human | |
| 403 | cg11387509 | CGCATCCGAATATGAGGCATTTTTATGTGAAAATGGAAAACCCGGCCAAG | Dog | | | | Human | |
| 404 | cg05094833 | CTACGAAAGCAGCCTAGCCTCCGCATCAATATTCATAAGGCAGTTTGACG | Dog | | | | Human | |
| 405 | cg05406595 | GAACTCACTATTCGGGATTCATGCTGGACATGTCACTGCAGCTGCCGCCG | Dog | | | | Human | |
| 406 | cg05746092 | CGGGACAGCATGATGATTGAAGAACTCCTTGTGCCATCCAAAGAGCAGGT | Dog | | | | Human | |
| 407 | cg05954117 | CGGGCCCAGCCGACGCCAGAAGGTCCCAACAGCAGCTGCCCAAACAGTTT | Dog | | | | Human | |
| 408 | cg06190403 | CTCCTACCTCTTCCAAGCAGCATGGTGTGAATGTCAGTGTGAATGCATCG | Dog | | | | Human | |
| 409 | cg06200735 | CGGCTCAGCTAGCAGAGCTAATAGGCTTCTAATTAATTTCCAGAAGATTG | Dog | | | | Human | |
| 410 | cg06254866 | GAAGTCTTTCTTCAGCGGCTTTCTTCTCACCCTGTTCTCAGTAAAGATCG | Dog | | | | Human | |
| 411 | cg06391736 | CGGCGTCGTTAAAGAGCAATTAGATGGAAATTAAGCCGAGAACAAGTATT | Dog | | | | Human | |
| 412 | cg06479220 | AGATACAGCGAGCTGGTGAATGGGGCACCATCTGCGATGATGACTTCACG | Dog | | | | Human | |
| 413 | cg06670018 | GCATAGTTTTTAGCATAACATCTTGACTCCTCGATAATTATATCCGTTCG | Dog | | | | Human | |
| 414 | cg06801233 | CGGAGGCTAGTTCCTGGTGGGTCAGCATGTACTTCTCACACTTAGCTAGT | Dog | | | | Human | |
| 415 | cg06940357 | TAAATACGATTTTATCAGCTCGGAATCTGTTGAAACACATCCATCTAGCG | Dog | | | | Human | |
| 416 | cg13264741 | CTTTTCTGAATTATTTATAGACCGGTACCAGCTGTTTTCAATTCCTCTCG | Dog | | | | Human | |
| 417 | cg07158563 | ATGTGCCATCACGCTGTCTGTTGCTTGAACTCCTGGATGTCAGTGTTTCG | Dog | | | | Human | |
| 418 | cg07211437 | AGACAAATGATTCGAGTCTGAATTCCATATACACCGGAGATTGCTTATCG | Dog | | | | Human | |
| 419 | cg14778330 | GCCCCCAAACCAGCTGTTTACATACAGGAATCAGGTATTGGGGCCCCTCG | Dog | | | | Human | |
| 420 | cg14956305 | CAGATGGTCCCTCCTAAAGGGGCCTACAACGTGGCTGTGATGTTTGACCG | Dog | | | | Human | |
| 421 | cg14956645 | CGAGTCGTTGGGAGAGAGGGCTTTGTAGGTTATAATAAAATCCTTTGAAT | Dog | | | | Human | |
| 422 | cg15436852 | TGCTGAAGCACCTGGTAGACAGGTACAATCTCTACTACGCCTACCTGCCG | Dog | | | | Human | |
| 423 | cg08712844 | TGCTGCCTGTCCTGAGAAATTTATGTCACACCCACTCCATCATCCTGGCG | Dog | | | | Human | |
| 424 | cg09595115 | CGGGCCAGTCTCGGGTTCTCGCTTGTAAACTTTATTGTAACTCTTCCGCC | Dog | | | | Human | |
| 425 | cg09689231 | CGCAAACATATTTCCTTGGTGAAATGTCAAAAAGGCAGCAATCAAAATGA | Dog | | | | Human | |
| 426 | cg15411810 | ACAAAGAGCGAGTGTGGGCCGCGGTGACTTCATGCCTCACCAATGTCCCG | Dog | | | | Human | |
| 427 | cg15436708 | TTTACAGCCCTATAAAAGCTGTAGCGACGAGGCAAGCGCTCCTACCACCG | Dog | | | | Human | |
| 428 | cg09973880 | CGCAGGCACTAGGTTATATAACAGTCTGAATGGAAAAAGGAAGGTCAGAG | Dog | | | | Human | |
| 429 | cg09996908 | GAAAATAAGACAGCCATTGTCCTCGCACGCCAAACGAGGGTTTGATGTCG | Dog | | | | Human | |
| 430 | cg10230808 | CGGCTGATGGATATGAAAAAATAAATTACTGTGGCAAAATGTAATAAACA | Dog | | | | Human | |
| 431 | cg10820794 | GACCCTTAATTACTTGCTAAATTAGAATGGTTTTCTGAAAATCACAAGCG | Dog | | | | Human | |
| 432 | cg17619764 | ATTTCACAGCTGAGCTGTGTATTGCCAGATAAATCAAATCAGGGTAAACG | Dog | | | | Human | |
| 433 | cg11160066 | CGCCTGCAAGTTCAAATGCGGGTTGTGACACCCATCATCATTATATCACT | Dog | | | | Human | |
| 434 | cg11242021 | TTACTGCAGCTAACTTCATAGACGTGATCATCACCCGGCAAATTGCCTCG | Dog | | | | Human | |
| 435 | cg11275402 | CGTGAGAGCCAGTTGGGAGAAACAAAGTTGTGCAGGCCAAGGGCATAAAG | Dog | | | | Human | |
| 436 | cg11339341 | CGCAGAACGTAGTGTGGGGACTGAAAAAGACCGTGGCAATGGATGAACAA | Dog | | | | Human | |
| 437 | cg11396061 | CGTTTCCAAACTGAATAGTCATTGGGCTGCCAGATAATTCAGAAACTATC | Dog | | | | Human | |
| 438 | cg11688874 | TGTTTCCTCGCATACATTACCATCAATGCCCGGCCTGTGAGTGTGTGTCG | Dog | | | | Human | |
| 439 | cg11762593 | AACGTGACCATGTGGATTCAACAGCTTTTAGGACTCAGGTGAGCGACCCG | Dog | | | | Human | |
| 440 | cg11900069 | AATTGTAATCAAGCCAGGCCGTCTCAGCTGGCTGATTAATGAGACCCACG | Dog | | | | Human | |
| 441 | cg11983139 | ATCCCCACCATTTGAGAGCTCACCTTGCAGCAGACAGACATGGTGGCTCG | Dog | | | | Human | |
| 442 | cg12026817 | GCTTAAGAATACGCGTAGTCGAGGAGAGTACCAGCGGCAGGGGGGCAGCG | Dog | | | | Human | |
| 443 | cg18888015 | CGCCAAAGCGCACGGCTCACAAAGGGAGCAAGTAGCTGCCACAGGGAACT | Dog | | | | Human | |
| 444 | cg12238798 | CGCACGCACCTCCCTCTGTGTTGTAGCAATAGGCTGTGTAGTGTCCTGAG | Dog | | | | Human | |
| 445 | cg12250639 | CACTCGCAGTGGTAGCCAGCTTCTATCAAGCCATCTAGAAATGCCCATCG | Dog | | | | Human | |
| 446 | cg17860485 | CTTCCCCTTCGAGGCGCTGTCTCAGGGGCAGCAGCTGTGCAAGGTGCGCG | Dog | | | | Human | |
| 447 | cg12464120 | AGGGGCCCCCGCCCAGGATGGACTGGATGGTGAAGCTGGAGACGCCACCG | Dog | | | | Human | |
| 448 | cg12525832 | CATTACAAAACTCTAATATACACACAGGGAAATTTGATGATTTGACTGCG | Dog | | | | Human | |
| 449 | cg19437021 | CGGCTGGGTGTTATTTAGTGCAATATGAAATCAAATGATCCTATTTCTCA | Dog | | | | Human | |
| 450 | cg12783911 | CGGTTTAAGTCATGTGATTGCAAATAGTAAATTAGTTTCTCGATGTTTTC | Dog | | | | Human | |
| 451 | cg12791206 | GTACCAGGATTATCCCGGGAACTTTGACACCTCGTCCCGGGGCAGCAGCG | Dog | | | | Human | |
| 452 | cg24262627 | CCCCGGGACGAGGTGTCAAAGTTCCCGGGATAATCCTGGTACATGATCCG | Dog | | | | Human | |
| 453 | cg13241538 | CGCCAGGAGCTGCACCGCATTATCAATGACCGGGAGATGAGGGACGCCAT | Dog | | | | Human | |
| 454 | cg20355078 | GCCCGTCTCTTGTCTTTCCTGGTTTATGACTTTCGGCTTTGTGGAATACG | Dog | | | | Human | |
| 455 | cg13326443 | GTACCCTGTGACAATTGGAGGCAAGATCGTGGGCTCCTTGTGTGCCATCG | Dog | | | | Human | |
| 456 | cg13487121 | GATGGCTTTGGGGGCCTTGCTTCCATTTTCCATTATTATGTGGACTACCG | Dog | | | | Human | |
| 457 | cg13499073 | CGGTCGCTAGGAGGCAGAACAAGATCAAGAAAGCTCAGCGAACTTGAACC | Dog | | | | Human | |
| 458 | cg13528652 | CTGGCTGGTTGTACCCATCAGCTTTGCGCTTTCCTCCTACATTTCCACCG | Dog | | | | Human | |
| 459 | cg18994669 | TAAAGCATTATCTTTGGTACAAGTACACACGGCAGGGCATTTTGGCTTCG | Dog | | | | Human | |
| 460 | cg13588645 | CGAAAGGGAATTAACCCCATCACCAGCCCGGACACCGGCTCCAGCGGCAG | Dog | | | | Human | |
| 461 | cg14441435 | CGAGATGGCCGACAGCCCCGAGTTCACCAGCATCCCTGCCTGCTACTGGT | Dog | | | | Human | |
| 462 | cg14456595 | CGGGAGCTTGTTAAATGTTCCTCATTATACATTTCTGGTCTGTTTCATCA | Dog | | | | Human | |
| 463 | cg14503550 | CGGCCAAATGTACTGCCCCAAAGAGCAGGCGCAGCCTCCCCACCTCTGGA | Dog | | | | Human | |
| 464 | cg14523578 | GTTTTTTCAAAAAGACGATGTCTTTCTCCTTGTTGCTTTTTAGTAGGTCG | Dog | | | | Human | |
| 465 | cg27633952 | CGGCTGCAAACCAGGTGTTTTTTTCTGAGGCTGTGTTGACAGCTGCTAAT | Dog | | | | Human | |
| 466 | cg21948411 | CGCTACAGTAATCCAGAATGTGTATTTCTGCAGATGATTAAATCAATACA | Dog | | | | Human | |
| 467 | cg22059823 | TGGGGCAGGCATGCGGCCACTCCATCCTCTGCAGGAGCCAGCAGTACCCG | Dog | | | | Human | |
| 468 | cg22159998 | CGCAACTTATATCTGTTGTCTCAAACAATAGGCTGGACAAGTAAGCCTCA | Dog | | | | Human | |
| 469 | cg15155511 | CGCAACCACCTGGAGAACAAAATCCATCTGTCTGACTGAATCTAGTTGAA | Dog | | | | Human | |
| 470 | cg22582760 | CGCCCGGGCAGATCTGCAAGTTATTTGAATGTATTATTCCAGTACCTGTC | Dog | | | | Human | |
| 471 | cg21098134 | ATTCAGCTTTGGCACCTTGGTATTAAATTTGCATAGCAGGTCACGCGTCG | Dog | | | | Human | |
| 472 | cg15478432 | CGACGACCCCACCAAGCAGACCAAGTTCAAGGGCATGAAGAGCTACATCT | Dog | | | | Human | |
| 473 | cg15618712 | GCTGGTGCTGAGGCTGAGGCTGGGCGTCCTCATGATCGCCTTGACTAGCG | Dog | | | | Human | |
| 474 | cg15652120 | ACTGCATTATTCCCCAGGCGGTGTGCAGTCTGTGTAAACAGGCTTTTCCG | Dog | | | | Human | |
| 475 | cg23095099 | CGAGGCCCCCAGCAGTGCCATCTCTCTCAGGGACGTCAATGTGTCTGCCT | Dog | | | | Human | |
| 476 | cg15686802 | GCACTTTCCGAGGGCGGCTGTACTTGACTGAGCTGCAGAACAGGCACACG | Dog | | | | Human | |
| 477 | cg15856582 | CGCCATCGAAATGACTGGAGCCTAAATCCAGCGCCTTAGACCACTCGGCC | Dog | | | | Human | |
| 478 | cg23693668 | TTATGGGGAAATAACTGTACACTTCTAGCATATTCCTGGCCTCCCTGGCG | Dog | | | | Human | |
| 479 | cg16066439 | CGCATGCCAACAACTCGTCCATCGAGCTGGAGAAGCTGGCCAAGAAGCAA | Dog | | | | Human | |
| 480 | cg16097144 | CGCCCGCCGACCTGCACCTGTCTGTGAAGTATGTAGGGTGGGCAGAAGCC | Dog | | | | Human | |
| 481 | cg16170567 | CGGGGATTGGAGTAGACGGTCAAATTAACGTCCATAGCTCCATTCTGTCC | Dog | | | | Human | |
| 482 | cg16184138 | CGGGAAGAGAATAATGTACTTTTTTATTAAAATGGAAATGACACACTGCT | Dog | | | | Human | |
| 483 | cg16288125 | TATTAACTATTCTGTGCTGTCCTCTTGGATGATAAGATCCGGAGTCATCG | Dog | | | | Human | |
| 484 | cg16754079 | GCGGGCGGTGTTTTTCCCCCTTTAATCCGAATTCACGGGTTTTCCCTTCG | Dog | | | | Human | |
| 485 | cg16793862 | CGCCCCCTCACCCTGGGGCTCCCCACCCGCCCCAGGTGATGCTGCTGCAC | Dog | | | | Human | |
| 486 | cg16882804 | TGAGCAGCTTACGACACTCAGTGAGCAGTAGGTGCCTGTGCAAGCTCGCG | Dog | | | | Human | |
| 487 | cg18678463 | TGCGGTAGGTCAGGTTGTCCACCTTGAGGGAGGTCATACCCTCCACATCG | Dog | | | | Human | |
| 488 | cg17410330 | AATAAATTATCGCTGACACTCCAGTATTTAATTATGCAGCTGTCAGAGCG | Dog | | | | Human | |
| 489 | cg17509850 | GGAGTCGGAGCTGGCGCTCTTCCAGATGTCCGTGCTGTGGCTGGAGCACG | Dog | | | | Human | |
| 490 | cg27029911 | CATTCTCTTCACAGTAAACCTAAGAACACTTGTAACAATTGCCCACAGCG | Dog | | | | Human | |
| 491 | cg17757479 | AGACGGGCCCAAGCCCCCCTTTGAAAGTGGGCATGAATCACAAAGCCCCG | Dog | | | | Human | |
| 492 | cg17957186 | CTACAGGTGCATCACCTGCGCTGTCCTTGCTGGTGGTCACTATAAGAGCG | Dog | | | | Human | |
| 493 | cg17980874 | CGCTGAAGTCCCACACCATGGCCCACGGCATCTTGGGCAGGTCGCACAGG | Dog | | | | Human | |
| 494 | cg18301087 | GTCTGCCGCAGATAATGGGGAATGCCACCATTCCTGGAGCCATATGAGCG | Dog | | | | Human | |
| 495 | cg20271991 | ATGATCTCCTAAATAGTGTCTGTAAATAAAGTCGCCTCTAATGGCAGGCG | Dog | | | | Human | |
| 496 | cg24466113 | CGCAGGGCGGCGGCAACAGCAGACAGACCCTGCTCAGCAGCCACCACAAA | Dog | | | | Human | |
| 497 | cg18797081 | CGGGAACAGCTGGAGATGACAGATCAATGGTTCAATACTCTCAACAGCAT | Dog | | | | Human | |
| 498 | cg27311945 | TACCATTTCTTTCCCATTTTTTGGCCAGAAGGTCATGTGCAAATTGAACG | Dog | | | | Human | |
| 499 | cg26364230 | CTGCCTCCCTCATAAAAGCGGGCATCTGTGGAAAGCAGGTTTAGTTATCG | Dog | | | | Human | |
| 500 | cg19382448 | TGTCAGTCTTTTGTATTTCAGCAGCTGGATTCATTGCTACCATGGTAACG | Dog | | | | Human | |
| 501 | cg19427611 | ACTTCCTCATTGGTATGCCAGCATCATGGATCATGTCAGCCACAGCAGCG | Dog | | | | Human | |
| 502 | cg15852492 | GTGACAAGAATGTCCAGCTCCTCCTTGGAGAACTTGGGCTTCCTCTTACG | Dog | | | | Human | |
| 503 | cg19477942 | CGCCTCCACCGAGGAGGGGATAATGCACTTGAAGACCGCAACATTGCCTC | Dog | | | | Human | |
| 504 | cg21750779 | TGCTGTCTCGGACACCCTGCTGGGCTCTTACCCTGTTGCCCTGGTAACCG | Dog | | | | Human | |
| 505 | cg17794466 | CGCCAGCATTGTGTATAGTTACCATGGAAAGAGTGAGTCAAGTGAAGGGC | Dog | | | | Human | |
| 506 | cg20007005 | CGGTCCCTGATACAGTAGTCTGGAGAATTCTCAAAATACACGAGGTCATT | Dog | | | | Human | |
| 507 | cg20349210 | CGTCGGAAACAGCCGGAAAATAAACACCCTGATGACTAAACGGTTTCTCC | Dog | | | | Human | |
| 508 | cg09313626 | CGTGGAACTCTCCACCAAGGGTCAGAAGAAGGGGCCTGGCCTGGCTGTCC | Dog | | | | Human | |
| 509 | cg22183626 | CGGGAGCCCAGGGACCGCTCCATCACTGGCAGCCATGGAGCCCGAGCATT | Dog | | | | Human | |
| 510 | cg20632980 | CTGGATGATGTCGTTGTTGTTCTCGCCATTTGGGGACAGGGTTTGGGTCG | Dog | | | | Human | |
| 511 | cg20761338.1 | CGTTGGACTCCTGGGCAATCCTAATTGCCTCCTGCAGGGCGAGCTCTGCC | Dog | | | | Human | |
| 512 | cg26932230 | CGTGTTCTGCAGTGCAAAGGGACTTCCCATGGCAACAACAAACTCCCCTT | Dog | | | | Human | |
| 513 | cg21149585 | GGGAGTGACCTTCAGAAAATATATGAAGCAAATGCCAAAAGGTTTTCTCG | Dog | | | | Human | |
| 514 | cg21178109 | CCTGTACAATAGGGTTGTGCAGGTTCTTCTGAACTGGGCCGGGACTGTCG | Dog | | | | Human | |
| 515 | cg21222571 | CGCCAGCGCCAATAAATAAGCCCATTAACCGGCGAAGTTCGAGTTTACGA | Dog | | | | Human | |
| 516 | cg21258436 | GGACTTGTCAGAATTCATTATTGACTGCGAAAGTCACCACCCTCTTTCCG | Dog | | | | Human | |
| 517 | cg21570151 | CGCCGAGGCCGAGTGCCTGGCCGAGAGCCACCAGCACCTCTCCAAGGAGT | Dog | | | | Human | |
| 518 | cg21608404 | GCTTTCTCAGGTTTCTGCTGATCTTGCAGCGCCCAGAAATGGACCGAGCG | Dog | | | | Human | |
| 519 | cg21698790 | CGCCGTCAGCACAACAAACGCACAACACAAGGAGAAACTTGGTGTCCAGG | Dog | | | | Human | |
| 520 | cg21741768 | GCAGAATTAGAAACAATCAGGGAGCTGCCTAATTACTGTCAATTAGAGCG | Dog | | | | Human | |
| 521 | cg21930209 | CGGCTCAAGTTCAACGAGTTCGTGAACACGGTGCCCACCATCGGCTTCAA | Dog | | | | Human | |
| 522 | cg01796316 | CAGATACTAGTCAATATCAAATCATGTAGATGGCGGCATTTTAGGCCTCG | Dog | | | | Human | |
| 523 | cg19912855 | AAATGGAAACAATTTTCATTCATTGCAATAACTCACTGTTCTGGAGATCG | Dog | | | | Human | |
| 524 | cg27569752 | TGTTTCCATAGCTATCCATATAAAAGAGCCCTTGGAATGAGGCTGACTCG | Dog | | | | Human | |
| 525 | cg22443103 | CGGGGTTGTAAAAAATACAGATGCCAAATTAACTGGCTGTGGCACTTGGC | Dog | | | | Human | |
| 526 | cg12774550 | TACCTTCAGAATCTCATACATGTAAAATCGAATATCATAGTCTGTTAACG | Dog | | | | Human | |
| 527 | cg18502095 | GATGCCATCCCAGGCAAAAAAGGCACTCGTGTTCTCATTTGGAACATCCG | Dog | | | | Human | |
| 528 | cg22599383 | GAGCAGCTTGAAAAGCAGGTCGTACGTCTTCTTCGCCATTGGGAGGAGCG | Dog | | | | Human | |
| 529 | cg22718540 | GCCTTTTGTTCCCAGCCAGAAGTTTGCATGCCGGGACCGTGACAGCTGCG | Dog | | | | Human | |
| 530 | cg22837949 | TGGAGCAGTGGTGCCTCCTCAAGGAGCGGCTGGGCTTCTCCCTGCACTCG | Dog | | | | Human | |
| 531 | cg23052275 | AGTCTGTTGTTGTTTATGCCGCAGCGCATAAAAGCTGTTAGCATTTTACG | Dog | | | | Human | |
| 532 | cg20848949 | CGCCCAATCGCCGTATACTCCCCGCATTTAACTTGGATGACATTTTGATT | Dog | | | | Human | |
| 533 | cg23108415 | GTTGTGCATTCAGCTTTTTAATGTGTTCAAAGAGCAGCTCTGACGCATCG | Dog | | | | Human | |
| 534 | cg07588584 | CGGCGCTGGGTCTTTTTTCATAGAAAAAGGTATTAACACATAAGCATCTG | Dog | | | | Human | |
| 535 | cg23777581 | CGGCAGCTGGCCAAACTGCTCTACGTCCACATGTTGGGCTACCCCGCCCA | Dog | | | | Human | |
| 536 | cg25882973 | CGCAAAGGTTACCGGATCCAGGCTGACAAAGAGAGAGACTCCATGAAGGT | Dog | | | | Human | |
| 537 | cg23934404 | CGAGCCGAGGGAGCCCCTTATCCCGGTGACAGAATGGGACAAGCTGGGAA | Dog | | | | Human | |
| 538 | cg24059119 | CGCCTCCCGCGCCTGCCATTAGAGGCGACTTTATTTACAGACACTATTTA | Dog | | | | Human | |
| 539 | cg26105915 | CGATGGTGCTTTGCAAGGATGTATTTATGTTATAATGGCCAACATTTGGT | Dog | | | | Human | |
| 540 | cg26297738 | GTAAATGGAGTTGTCTCTTTAAAGTGCCCCGGTCAGTTCACCACACGTCG | Dog | | | | Human | |
| 541 | cg24512174 | CGCGGCGCCATGGGCTGAAAACTCAACCGAGATGGAATCTCCCAGTCTGA | Dog | | | | Human | |
| 542 | cg24905433 | CGATCAGATGAGTTCCTCTTAGTGCAGGTCAAAAAGGCTAGTTAGCAGGA | Dog | | | | Human | |
| 543 | cg25621619 | ATCACTGGACAAGGTAACAAGGGCCGGAGCAACCCGCTTGTCAACTAACG | Dog | | | | Human | |
| 544 | cg25337652 | CAACACTGTTTTGCCCGATGTAATTGCAGTTTCCATATAAATAATTCACG | Dog | | | | Human | |
| 545 | cg27132917 | TGGTGTCTCTGGCCGGGGACTGGAATTTCATCTGAATGACTGCCCCTGCG | Dog | | | | Human | |
| 546 | cg25520488 | CGGACTCTACCTGTGGCTCAGGCATACCAGGACAACCTGTACAGGCAGCT | Dog | | | | Human | |
| 547 | cg07158748 | CGTGCAAGGCAACCACACTGACTGTGTACTCAGAACCCGGTCTGAGGCCT | Dog | | | | Human | |
| 548 | cg27290535 | CTGAATCAAGCCTTCTAGCCTCCCCGTGTGACGAGTGCAATAAGGTAACG | Dog | | | | Human | |
| 549 | cg25877801 | CGCATGCGAGAGCTCTGTGCGCTTTACAGGACCTCTGGAAGCAACTACTC | Dog | | | | Human | |
| 550 | cg27376149 | CGGTGACACGATTTCTAATGAGCTGCTCAGAGTGCCAGAAAAGAATGCAT | Dog | | | | Human | |
| 551 | cg27290859 | TGCTTTCAATTCATACTGCCAGCTCTACCTCATGCCATTGACCTTTTACG | Dog | | | | Human | |
| 552 | cg27043849 | ATGGGTCACCGACCCGCGGGTGGAATGGGCGGCCTGGGGTTACTGGGACG | Dog | | | | Human | |
| 553 | cg24173251 | CGGGGCCTATGTCTATCCCCCGCCAGTCGCCAATGGAATGTACCCCTGCC | Dog | | | | Human | |
| 554 | cg24244412 | CGTGTCCCTACTGGACTTTTCAAGGGTCAAAGAGCAATGCTTCAATTAAT | Dog | | | | Human | |
| 555 | cg05661521 | CGGCACGGCAACCTTTGCACCACCCACTGCACCTTAAAAAGGGGGAATGA | Dog | | | | Human | |
| 556 | cg13226074 | CACATCTGGTTTAGCTTTAGACATTTTCTGGGATTTTGCCAGCAGCTACG | Dog | | | | Human | |
| 557 | cg07104049 | CGGCTCAGAGCTAATATTATGGCAAAGTAGTAATTATTTCAACAGTAAAC | Dog | | | | Human | |
| 558 | cg18740802 | CTACTAGGCCTGTCATTTCAGCATCCTTCCATCATAACATTGTGGCACCG | Dog | | | | Human | |
| 559 | cg25283972 | GTAACCTTGTGGCGTATTTAATGTAAATAAGGCAGTTATGTTGCCAATCG | Dog | | | | Human | |
| 560 | cg05383437 | CAGTCTTAGAATGAGTAGCAATATCCACGCGAACCATCTCAGCCTAGACG | Dog | | | | Human | |
| 561 | cg00773909 | CGCGCACCAAGACCGTCCTGACATTACTGTTTTATGACCTTGACTTATTG | Dog | | | | Human | |
| 562 | cg17026220 | CCCGGTTGTTTGCAGAAAATTTACAGCTGAGTAATAAAAGTTTACGATCG | Dog | | | | Human | |
| 563 | cg14727354 | GGGGAATAATGCCGTTATTCCTCGGCTCTCAGTTAATGAATACCCTAACG | Dog | | | | Human | |
| 564 | cg21225398 | CGGCCATGACATTAAAAGAGGGGAATGTGTTTTGGCCAAAGGAACCCACA | Dog | | | | Human | |
| 565 | cg17997463 | CGGCTGTGGAATGGCCGCCAAAATCCCAGCTGTGCTGCCAGAACCTAATC | Dog | | | | Human | |
| 566 | cg01460805 | TGGAGCCATGTGCGGCGTGGAGTCCCCGCGGTGGGAGAGGTACTGCGACG | Dog | | | | Human | |
| 567 | cg01322554 | CGGACTTTCCAGCCATCTCCAGCTGTGAGATTATTATAGTTTTTGCAAAC | Dog | | | | Human | |
| 568 | cg02445202 | CGGAGATCTGTGATTTTCATTAAGATCTTTGGAAACATGTGAGGCTTGCT | Dog | | | | Human | |
| 569 | cg01434367 | CGTGCACACACAGCTCCTAATTAGGGGTTGCTGTGCAGTAAACAAAAATG | Dog | | | | Human | |
| 570 | cg18408412 | CCCCAAGTGTGGAAGGTGGTTCTTAGCTGTCAATTCCCTTTTGTTATTCG | Dog | | | | Human | |
| 571 | cg20010041 | CCAGAGTGGAAGTTCCATTCAGGGGGCTCTAAGGCGGCAACCCTCCATCG | Dog | | | | Human | |
| 572 | cg14783226 | GCAGCACGGCTCCGCGGACTTTTTTTCAAACTCCCATCAATGAGACTTCG | Dog | | | | Human | |
| 573 | cg02574410 | CCGCTCCCCCGTCTCTTCAAAATGGATGAATCAAACCAGCCGAAAATGCG | Dog | | | | Human | |
| 574 | cg00296428 | CGGATGCTCAACCTCACTGACCGGCAAGTCAAAATCTGGTTCCAGAATCG | Dog | | | | Human | |
| 575 | cg17189172 | CGCGCTGCAGATCTTCCCCGAGGTGGCCGCCCAGGAGGAGATCCTCAGCA | Dog | | | | Human | |
| 576 | cg07236812 | CAGCAGCCCCGCAGCAGGGAGCTGATGTTGTAGATGGGCTGGCCCTGGCG | Dog | | | | Human | |
| 577 | cg15408129 | ACGCCTTCTACCTGGAGAACCTGCACTTCACCATCGAGGGCAAGGACACG | Dog | | | | Human | |
| 578 | cg00915901 | CGGGTCGGGGGGGGGAAGCCATTTCCAATCTGCTCTCTATCATTTTCAAT | Dog | | | | Human | |
| 579 | cg07661299 | TTAAAATTCATGAATCTCTTCACGACCATTTGTGTAACAAGATTAAGACG | Dog | | | | Human | |
| 580 | cg01193422 | CGCGAGGTCACCGTAAGCTCCTTAATGGACTGTGAAATAACTTTAACTGC | Dog | | | | Human | |
| 581 | cg01333192 | AAGTATATGTGACCTAAAAAGGCATTGCTCATTCTGACACATATACGACG | Dog | | | | Human | |
| 582 | cg01342051 | CGGCATAACAACTATTTGAGGGTTTAATTAGAACATCTGCAATTAAGGGA | Dog | | | | Human | |
| 583 | cg12712815 | GCTTGGAGAGTTCGCAATAGCCTCTTTTCCCACAAGCTGGACCACACACG | Dog | | | | Human | |
| 584 | cg10520899 | CGCCCGGCCCAGAAAGGTACAATAACTGTATATTTTAACTTAATGGTTTT | Dog | | | | Human | |
| 585 | cg12753118 | CCATTTCTGGATTATATTTCAGCTGTAACTTTTAAGTCTAATTATTTGCG | Dog | | | | Human | |
| 586 | cg01938074 | TCATTCTGTATGCTTGACTATTTTGTTTTTAATTTTTCTTCCCTTTCCCG | Dog | | | | Human | |
| 587 | cg12938952 | CGGCAGAAATAAATCCATTCATTTGATCATGTAGTCATTGAGGGCGATAC | Dog | | | | Human | |
| 588 | cg26523389 | GCTGGAGCCTCTGATTGGGTTTCGGAGTCCGGTACTGGAGCCAATCAGCG | Dog | | | | Human | |
| 589 | cg20126035 | CGGGGGGACGAGCAGGCAACAAATCCAGGCCTCCGACATTACCAGAATGT | Dog | | | | Human | |
| 590 | cg02633867 | CGTCAAAATTCAATCACTGGGGGTTTTGCATCAGCCGGGCTACTTATTTG | Dog | | | | Human | |
| 591 | cg02737031 | CGAGCTGACTTGCAGGCATGAGAGATCATCAAGAAAGATAAAGAAATAGG | Dog | | | | Human | |
| 592 | cg21178573 | CGGAGAAAGCACCAATTGAATTAGTTGCCTATTTAGTATCCAAAGCGCCA | Dog | | | | Human | |
| 593 | cg03104256 | CGCTAAGCTCAGCTTGTTTTTTTCCTCTGCTCAACAGTTCTCCTGCCACG | Dog | | | | Human | |
| 594 | cg15343900 | CCAGAAAATAATAAACCTTCCTGGTGGGGTGCAGGGAGCCAAGGCATTCG | Dog | | | | Human | |
| 595 | cg18878759 | CTGCAGCCTGCAAAACGAATTTGGTGCTCTGAATGCACTGGTCTTGGTCG | Dog | | | | Human | |
| 596 | cg22619012 | TTACGACCCAGCAAGAGGCCGAGCTGGTACAATATCCTGACTGTAAATCG | Dog | | | | Human | |
| 597 | cg12026556 | GGTTTCTCCGAAAGCTTACGATGCAATTTCCCCCTCCAAAAAAAGAATCG | Dog | | | | Human | |
| 598 | cg14530571 | CGCTGCATTGGGATACAGATCCATCAGTTCTTCAGCTTCACTCAGATTCC | Dog | | | | Human | |
| 599 | cg05907933 | CGGCATGCTCCGTGCTGGGGAAATGAGATGCACATTTAAATCTCATCCAT | Dog | | | | Human | |
| 600 | cg12990321 | CGGCCGCTGCCCCCCTGCCGCTGGTACTCTCCTCGACTACGCGTATTCTT | Dog | | | | Human | |
| 601 | cg24516716 | CGGCACACTGGGTCCCGAGGAGTTCAAAGCCTGCCTCATCAGCTTGGGTT | Dog | | | | Human | |
| 602 | cg10523202 | ACTTGTACCAAAGATAATGCTTTATGTGAGAATGCCAGATCCATTCCACG | Dog | | | | Human | |
| 603 | cg21451202 | CTCTGTTTGACCAAGCGGCCAAGGTAAAGATCCTGGAGGTGATGCCTGCG | Dog | | | | Human | |
| 604 | cg06721070 | CGGATTTACGAGTGCAAAACATGTGGCGCCATGTTCACCAACTCTGGAAA | Dog | | | | Human | |
| 605 | cg17563106 | CGCTGCTTGTAGCTTCGTCCACAGTAGTTGCACTTGTGAGGTTTACCCAC | Dog | | | | Human | |
| 606 | cg18480320 | ACAGCTTCGCTTTCTGCAAACCACCTTTCAAAAATTTTCTTCTGGAAACG | Dog | | | | Human | |
| 607 | cg18877412 | CGCTTTCAGTTGGGCTGCTCGGTCTAATCTTATTTGTTTTACACCCATCA | Dog | | | | Human | |
| 608 | cg25199827 | AGTGCTTCTCGTTTCAGCAGCACAAGCTACATGTCTGTCCAATCAGAACG | Dog | | | | Human | |
| 609 | cg13570084 | CGATCAATAGCTACCAACATTTATGGCTTAGATTATTAATGTGAAAGCTG | Dog | | | | Human | |
| 610 | cg09370029 | CGCCCTAATTGATAGACAGAAGGCAAAGAGGAGGATTGTTTTTGAACCTG | Dog | | | | Human | |
| 611 | cg19008877 | CTACTGTTGCCAGTATGTTTTCAGCTGTGCCAGAACCGAGCAGGTCTGCG | Dog | | | | Human | |
| 612 | cg17489939 | ACCACCAACATTAAGGAAGAATCCAATTGCTGCATGTATTCTGATAAGCG | Dog | | | | Human | |
| 613 | cg02884952 | CGAATGCAGCTGCTCTTTGTAATTGTTTGTGAAACTGAGTTAAAGGGAGG | Dog | | | | Human | |
| 614 | cg09075948 | CGCTGTTACTGTAAATGTTCCCTCATTATCTATGTTATGGTAGGAATTTG | Dog | | | | Human | |
| 615 | cg08167680 | CGCCTTTCAAACGAGGGCAGTTTGTCATAAAAAACATCGTCTTCTGACAC | Dog | | | | Human | |
| 616 | cg25574114 | CTGGGAGACATGCTCGATGGATGATGGCTTTTAAAATATTCCTAATGTCG | Dog | | | | Human | |
| 617 | cg27643609 | GTGCTGATTTATTGCAGAAGCCTCTGGTCATTTCCATATATTGAAATGCG | Dog | | | | Human | |
| 618 | cg10162464 | CGGGGAACAGCACTCCCAGGATGCAGTTTGTGTCAACACGGCCGCAGCCT | Dog | | | | Human | |
| 619 | cg16390009 | CCCATGTTTATCACCGAGTGAGATGACTAATTACACCTTGTCAATCACCG | Dog | | | | Human | |
| 620 | cg18977436 | TAGGAAAAACGATAATATTCCGGATCTTCTCCCAGTTTTTTCCCCTCACG | Dog | | | | Human | |
| 621 | cg26375445 | CAAATGTGGCTGAAATTGATAATTCCAAAGTGCTTCATTTCGGCTCCTCG | Dog | | | | Human | |
| 622 | cg13160963 | CTCCATGATCAACCTGTCGGTGCAGCAGGTCCTGAGCCTCTGGGCCCACG | Dog | | | | Human | |
| 623 | cg24142628 | CGCGGGTTTCTCGAGGCCTGTTTGTTATTAAGATGTAAATTAAATCATCA | Dog | | | | Human | |
| 624 | cg14044498 | CGACGTGAATGAAGCCTCATTTCTGGCCTTGAGGAGCTTAAGATGTATGT | Dog | | | | Human | |
| 625 | cg07224103 | AATTCACGGGTTTTCCCTTCGCTTTAATGGGGGCCACAGGGACCAGCTCG | Dog | | | | Human | |
| 626 | cg14387950 | GGAAGGAGCTGCGAAAGCCCCCCTTCTGGTCTTGCCTGGGATTTTGATCG | Dog | | | | Human | |
| 627 | cg26647139 | TCCCCGGATTGACAAAACAATCTGGGAGAAGTGCATGCGTGCTTTGCACG | Dog | | | | Human | |
| 628 | cg20314902 | CGGCGGAGTGTCCGGGTGAAAACCAAGGTACCTTGGATGCCCCCTGGAAA | Dog | | | | Human | |
| 629 | cg05066539 | CGCTGGAGCTCCTACATGGTGCACTGGAAGAACCAGTTCGACCACTACAG | Dog | | | | Human | |
| 630 | cg25040028 | GGTCGTAAAAATCGGCTGTCTCTGTAAGTTATCGTATCCGTAAAACTTCG | Dog | | | | Human | |
| 631 | cg24517026 | CGCGCGGTACTTATACAGCAGTACATGCAGAGCAATGCCGAGGTTGTGAG | Dog | | | | Human | |
| 632 | cg07196802 | CGCCGCCTTCATGGACCGCCTGGGCCACCAGCTGGAGGACATGCTGCTCT | Dog | | | | Human | |
| 633 | cg10711876 | AGATGAAGGACGATGTCAGACTCATTTTCAGCCTCATTAGGCAGCAGACG | Dog | | | | | Universal |
| 634 | cg17215796 | GCTAGTACCAACTGCTTGAGACCAGATACTAGTTTGCATTCACCAGAACG | Dog | | | | | Universal |
| 635 | cg27507315 | CGCCATTCAGGCCAGGTCCATTCTGTCATTTATAAAGATAGTTTCTTCTG | Dog | | | | | Universal |
| 636 | cg16437032 | CATCTTTCTAGCTGACTGGCAACAATCTGCCTTTCCACTTCCAGTTCTCG | Dog | | | | | Universal |
| 637 | cg16636772 | CGCCCGCATTTTAGGATACAGGAACAAACACGGATGTGAAATCAAGAATG | Dog | | | | | Universal |
| 638 | cg19111172 | CAATACATTCTATTTAGAGTGCATTACATCACCCCCCCGGTGACGTCACG | Dog | | | | | Universal |
| 639 | cg15713630 | CGGCTTGTTTACTCAGGATTGGGCATTAGAACTCAGGGGAGGCTCTAATT | Dog | | | | | |
| 640 | cg06561106 | CGCTCCCCCGCCGAGCTGGGGTAGCTGATCACTGAGCTGAAACTAAACGT | Dog | | | | | |
| 641 | cg24954807 | TCAATTTTATACGGTCATCAGTCAGGTTGTGAATGCAAATACCTCTGTCG | Dog | | | | | |
| 642 | cg04523259 | CGGCTGCCCAACCTGCTGGGCCACGAGACCATGAAGGAGGTGCTGGAGCA | Dog | | | | | |
| 643 | cg02916332 | GGAAATGCAATAATTTTGCCATAATGGGCTGTAACCTCAATTCGACCCCG | Dog | | | | | |
| 644 | cg13288931 | ACCTTGATTTAATGTCATACAATTAAGGCACGCGGTGAATGCCAAGAGCG | Dog | | | | | |
| 645 | cg09256836 | CCTTTCATTGTCACATTAACACCAGATGCTAAAAATAAGCCTCCAAACCG | Dog | | | | | |
| 646 | cg14935662 | GAAAATGCCTTTAAGGGACAAATACTGTCAGACTGACCACCATCATCACG | Dog | | | | | |
| 647 | cg10586546 | CGGAGCCCACAGAACCAGGAAATCCAGGACACCTCCTCTGAGCTGCTCAT | Dog | | | | | |
| 648 | cg15755108 | CGAAGACGAATATAGTTAAAATGTAATTAAATATGACGGGCTAGCAGGAT | Dog | | | | | |
| 649 | cg12375054 | CTTCCTGCTGGTGAACCAGCACAGCATGGTGAGTGTGTCCACGCCCATCG | Dog | | | | | |
| 650 | cg23743304 | TGCGACCTGCCGGTGGGAACTTTGTCTCCGAGTCGGAGCAGCATGGAGCG | Dog | | | | | |
| 651 | cg08489771 | CGCTCCTTTTGTATCTCGTGATTAAATAAGTGGGGAAAATGGCTTGAAAA | Dog | | | | | |
| 652 | cg20003348 | CGGCAAGATGATATGAAGTAAATCAAGGTACGAGTAATGAAAGACTGAGA | Dog | | | | | |
| 653 | cg20316422 | TATGAGGCAACCCAATCTTTCCTCGGATGGCACTGTTAAATTTTGGTCCG | Dog | | | | | |
| 654 | cg09228164 | GGTCGCTGGAGAGGTGAACCAAAGACACTTCCGTGTGGATTTGGACAGCG | Dog | | | | | |
| 655 | cg09243281 | CGGCATGATTTGGATATCATCAAACGGTTGTTGGACCAGTGTATTCAAGA | Dog | | | | | |
| 656 | cg17583255 | CTTTCGAGAAATGGCTGGGCCATTGTGCAGAAGAATGCCCGGAAATCCCG | Dog | | | | | |
| 657 | cg21645186 | ACGTACTTGGCATAGACATCCAGACTGGCTGTCTCCACAGAGATGTTGCG | Dog | | | | | |
| 658 | cg19654336 | CGGTCCCTCTCACCCGCCTATTGTGTACACAGTGAAGGTCATAGGTTCAA | Dog | | | | | |
| 659 | cg22234098 | ACTCCACATCACCCAGGCTTTGGTAAATGTAGCTGGTCGCTGTATAGTCG | Dog | | | | | |
| 660 | cg00013189 | CGGCGCTCTGAGGCCTCCCCATTCACCCGCCTTGGCACGCTCACAATCGG | Dog | | | | | |
| 661 | cg13804575 | GGAGCCCTGGGAGAAAGCCCCAACTGGTTGGTCTTTCCCCATCCCAGACG | Dog | | | | | |
| 662 | cg17443147 | AGTTAAAGTGCCACAGAGTTTGCTGGCTGCGCTTGCTGGTTGATTTAACG | Dog | | | | | |
| 663 | cg14435444 | CGGCGGAAGACGAGACAACACCTGGCAGCAGCCTGGAATCTGATTTGCTT | Dog | | | | | |
| 664 | cg21921063 | CGGGTATTCTTGGGTGGATAATACGGATTACGTTGTTATTGCTTAAGAAT | Dog | | | | | |
| 665 | cg02393105 | ACCTTAACTCGAGCTCATGTGTGGTAATTGCTTTATTCTGCATGAACTCG | Dog | | | | | |
| 666 | cg12735070 | AGTTTGAATTCCATCCTACATTAAGACTTCTTTCACAGCTATTTAGCTCG | Dog | | | | | |
| 667 | cg05779610 | CGTCTAAACAGGCGCCAAGTTGCTGGCAATGCGCAATTATTTCCTTGTCA | Dog | | | | | |
| 668 | cg21880134 | CGACCTCCGGGTGTTTAATAACCTTCAGAAGTTTCCCACAAAGGCTCAGA | Dog | | | | | |
| 669 | cg26616428 | CGGGTGACAGAGCGTGTGTTGAAGCTGAACTCTATAATTTGCGCTTCTGT | Dog | | | | | |
| 670 | cg19803918 | CGAGCATGAAAATTCGGTTTGAGCCGGCTGACAATACAATGAAAAGGCTT | Dog | | | | | |
| 671 | cg21730656 | CGCCTCTGCTTGCCTTTGATAATTGTGTGCTGACTACACGTGTAAAAAAA | Dog | | | | | |
| 672 | cg26743713 | CATTTCATTTCAAGGGCTCAGCCACTGGTCTGTCAAATCTTCATTTTGCG | Dog | | | | | |
| 673 | cg08164193 | CATCTGGAATTGCGCTGCGCTCACGGCCAAGGCCGATTGTTTAGTCTGCG | Dog | | | | | |
| 674 | cg00193345 | CGCTTTTGGCTCCTTCAGGAATGCGCTGAAGCTTGTAATCAGCTTTGTGA | Dog | | | | | |
| 675 | cg02124022 | AATTGACTGAGGGGCAGGTGAAGCTCCCGCCCCATGCAGGCTCATTCACG | Dog | | | | | |
| 676 | cg02857399 | CGTCGCTGCGCAGGTCTTTTTTGTTGGCCACCAGGATGATGGGCACATTG | Dog | | | | | |
| 677 | cg19608474 | CGATTGAGTTAATCTTTGAAGAAAACAGAAAATACTGTAACAATAAAGGC | Dog | | | | | |
| 678 | cg14001847 | CGGACGCCAGGAAGCACAGGGACAGGTGCAAGGCCTTCAACACCCGCCGG | Dog | | | | | |
| 679 | cg08664231 | CGCCTGGAGGGATCAACACAGTTTTTTAAAGCAAACATAACACAAGTGCT | Dog | | | | | |
| 680 | cg10437271 | CGCTCCGCCGCTCCATGCTGCTCCGACTCGGAGACAAAGTTCCCACCGGC | Dog | | | | | |
| 681 | cg26434265 | TAAAATCAGTCCAGCGCTTTTTTCTCGGCCCCTTCTTTATTGTTGGTCCG | Dog | | | | | |
| 682 | cg25790333 | CGGGAGGAGAGTTTAATTGGTTTAGCCTAAAAAGCCCATTACGCTCTCTG | Dog | | | | | |
| 683 | cg02800396 | GGGTCACCAATGGGCGCGGAAAGTCCTCACTCTCATGGCTCGGGCCATCG | Dog | | | | | |
| 684 | cg12190012 | AAATTACCCACAAAATAAGAACAGGAAGTAATTCAGCACAGCAGAACACG | Dog | | | | | |
| 685 | cg10546888 | CGTGATTACACCCAGATCAACGTAGGCAATCTGGAGTTGAGTGCCAACCA | Dog | | | | | |
| 686 | cg20918588 | CGGAATTAGCCCAGGAATTTCTGCTGATAAACAAATGAAACTCTCAAAGG | Dog | | | | | |
| 687 | cg24359120 | CTAGGGGGCATTTGCACATATATTTGTACTCAACATTTCATGGGAAAGCG | Dog | | | | | |
| 688 | cg20109856 | CGCTAACCATCACTAAACAGTGACAGTAATAACAGCTAATTTTGCTGGCA | Dog | | | | | |
| 689 | cg07189644 | AGCTAAGTTAACCTGATTTCCCTTAATTAAAACATCTTTTCTCGTTTACG | Dog | | | | | |
| 690 | cg22201025 | CGGGTCAGGCCAGCACAGACGGTTTGTACTTCCTTATTGTACATTTTAAG | Dog | | | | | |
| 691 | cg00688975 | GGAGGACTTATTCGGTTCCACGTCAGAAAGTGACACGTCAACTTTCCACG | Dog | | | | | |
| 692 | cg03103218 | CGGGGGACTTATTTACTGGCAAATCCATACTCATTACAAGACTCAGCCTA | Dog | | | | | |
| 693 | cg09574770 | AAAGCGCATATGCAAGCCATGAATTATCAACTGAAAGGAGTCAATTACCG | Dog | | | | | |
| 694 | cg20855763 | GCAGGTTGAGGCGGCGCAGCGGGCCCTGGTGGAGGTGCTGGGGCCTTACG | Dog | | | | | |
| 695 | cg25244901 | CGGGTGGGCTGAATGTGTTGTGAAATGGCGATCATTGCCAGAGATTAACC | Dog | | | | | |
| 696 | cg03472204 | TCGGCACCTGGTTCAAATTTAAGGCTCATTTACACATGATGTCACACACG | Dog | | | | | |
| 697 | cg24396429.1 | ACATGGTTTGCATAGCTGGATCTCAGCTTATCTGTGCCTTTCGGCTTTCG | Dog | | | | | |
| 698 | cg06031500 | CGGCGGACCATCAGCTGCCTCCAGAAACAAGTAATCAAGAATAGGCCTCA | Dog | | | | | |
| 699 | cg24956561 | CGTGCATTTTTATTCAGAGATAATTATGTGTTGATCAGAGAAGTCAGCGG | Dog | | | | | |
| 700 | cg23677945 | GTAGAGCACCATGAAGAGGATGGTGCAGCCCTCCTTCTTGGTGCCCTGCG | Dog | | | | | |
| 701 | cg09637123 | GAGAAGCCGATGTGTGAGAAGACCACCATCAACAATGAGTACAACTACCG | Dog | | | | | |
| 702 | cg23505448 | CGAGGTTAGAGAGGTGAGCAAAGGAAATAATTAACTCATTTGCAGCCTTT | Dog | | | | | |
| 703 | cg14842657 | TTCTAAATAGGCTTAAGGGCTCCATTTACATGAAAAGACAGACAGCTCCG | Dog | | | | | |
| 704 | cg05278305 | CTTTTGAAGAAACCTTTGCAACCTTCACAACTGACAGCCCCATAGTGACG | Dog | | | | | |
| 705 | cg25590275 | CTGAGGATGATTATTAATTAATCAAAAACACTGACTTTTAACTTCCCACG | Dog | | | | | |
| 706 | cg06126658 | AATAAAGTCATTCCACAAGTGACTAAATCTCTTAATTCACAAGGTACACG | Dog | | | | | |
| 707 | cg15031516 | TCAGCAGCTCGGCTGAAGTGGGCCCTTTGGGCTTGTGCACGGCGAACACG | Dog | | | | | |
| 708 | cg15587600 | GGCTTATTAACTCAATGTGAAGCCAGATTAAAATCTTTAATTATAAATCG | Dog | | | | | |
| 709 | cg07340783 | TGCCTAACATGATTCTTTCAGTCATTTATTATGCAGCTGCTACAACGTCG | Dog | | | | | |
| 710 | cg07687140 | CGAGTCGGTGGTTTGTCATAACAGTCATCAGTAACTGTCGCTTTCTGCCG | Dog | | | | | |
| 711 | cg24726068 | TAGATCATAGACACACGGTTCTTGATATACACATGATAGGGGTCACTACG | Dog | | | | | |
| 712 | cg27503226 | CGGGCTCTAATGCACACTGTTTTACACGTTAACGGTTTTGAAGTTCCAGG | Dog | | | | | |
| 713 | cg24383169 | CGTGCCAGCTTAGTGTCAGAGTGCCTGTTCGCCAGACGGCCACTCAGGCC | Dog | | | | | |
| 714 | cg17489311 | AAATGAATTTGACAGCAAACATCATAACAGTTAGCAGTAATGTGTGTTCG | Dog | | | | | |
| 715 | cg26419744 | TCGTCTGTATTTATTTAGTGCTCTGGGGGAGTTTTTCTCTTTACGCAGCG | Dog | | | | | |
| 716 | cg17247323 | TGTCCTCAGTCTTTTTCTGTTAGTTCATTACTACACAATTACCATTCACG | Dog | | | | | |
| 717 | cg01620164 | TTTAGCTCAATAGTCACCAGAGTCCAACCAGACGTGTATGTCGCGCAACG | Dog | | | | | |
| 718 | cg20694166 | CGCTCACGCTGTGTGAGCTCAAATCCTAGCATTAATGTACTGCTGGGTTC | Dog | | | | | |
| 719 | cg06759265 | CGGCCTGGATGGCCCGGAGGTAGCTGTGGCTCCGCATGCGGAAACAGCCG | Dog | | | | | |
| 720 | cg21577422 | CGTGCCCTCCAGTGGCTTTTCTGATTCGCTTTCTGACCTAAACACACATT | Dog | | | | | |
| 721 | cg07846083 | TGAATAAGGCCCTGATTGCAAGATCCTGTTTGGAAAATTATAGTGCGGCG | Dog | | | | | |
| 722 | cg10194942 | CGAGGCTGCTATACTGACTTCCATGTGGACTTTGGTGGTACCTCTGTTTG | Dog | | | | | |
| 723 | cg23024952 | CGCCACAAGCGACACAAATGAACACGCCTTCTCAGCCCCGCCAGGTGAGG | Dog | | | | | |
| 724 | cg13550064 | CGACGAAGGGTTTTGTTACAGCATGTGCATGAAGGCTATGAAAAAGATCT | Dog | | | | | |
| 725 | cg02707033 | CAAACTGACAGAGGGAACGGATGCTCTTTAGTAGCACATGCCCAGGATCG | Dog | | | | | |
| 726 | cg10818013 | CGGCCGGCTCAGCGAGACTGAACTAAAAGTCTCACACTTCATTGAACACC | Dog | | | | | |
| 727 | cg23116266 | CGAAGCGCTTTGTTCAGTGCAGCGTTGCAGAGGGGTTTTCCCAGGCTCCG | Dog | | | | | |
| 728 | cg24015091 | CGACAGTGAGAAGGAAGAAGGTGAAAAGTCTCCCAGCTGAATTCTTGGCT | Dog | | | | | |
| 729 | cg21879618 | CGCCTAAGGCAATAGGACTGTACAATCTGTTATGTACGAGGCTGTGGCTT | Dog | | | | | |
| 730 | cg07645671 | TTTTTTTCATGCTTAAGTCCAATGTGCCATTTTCATCCACTTCTATTTCG | Dog | | | | | |
| 731 | cg25236972 | CGCGGTGGTGCTAAGTTGAGTCATCAGTTCCCTTTACGCCCTAATAATTG | Dog | | | | | |
| 732 | cg07054032 | CGGGCAGCTGCTGATGAACTGGCGGGTGTAGCACTGGCCCCAGCCCTTCA | Dog | | | | | |
| 733 | cg15240113 | CGATGCCGAGTTCCAAGCACCATTGTTAATACAGACCAGAAGAGGAATTG | Dog | | | | | |
| 734 | cg08044276 | ATTTCAAATGGAAAATTAATGCTGAAAAATAGCCCTGAGCTCTCTTGTCG | Dog | | | | | |
| 735 | cg19537997 | CGAGCTAAGCTCCATTCAAGGCCAGGAAAGAAAATGAGCACAAATAAATA | Dog | | | | | |
| 736 | cg21989377 | CGCAGTACATGCTGTGGTTCTCCAGCTCGTGGTCTGTGCAGGTGGAGACC | Dog | | | | | |
| 737 | cg25671487 | CATCTCTTTTATCCTCGCTTATGAATAAGGTAAGAGCCATCCATCACACG | Dog | | | | | |
| 738 | cg09286000 | AATTTATTTTATTGCTTTTATACTTGGCCATTTCCTGTAGCACATTATCG | Dog | | | | | |
| 739 | cg02344882 | CGCTGGGCTCTGCCAAGCGTTGCCTCTGATAATGAGTGGGATTTGGTTAA | Dog | | | | | |
| 740 | cg26924415 | TGCATGGGGACAATGATGCTTGATGTGACAATTAGGCTTCTAAACACACG | Dog | | | | | |
| 741 | cg06035436 | GAGTTACAAGCTTTCTTCCACTGCCATCTGGCAGGTTTACTGATGGATCG | Dog | | | | | |
| 742 | cg17538927 | CGGCTCATTTACGGACTATTTAGGTCGTGCAGTGAGCCAGATGAGAACAG | Dog | | | | | |
| 743 | cg04478143 | CGGATGGGAGGCACTGGGGTCCTCTAGCTCGTAAGTCCTCATGAGACATC | Dog | | | | | |
| 744 | cg01248517 | AAGCGTCTGGATGCCGCACATTTACATCTAACAAGAGACCCCAGAAAACG | Dog | | | | | |
| 745 | cg16698149 | CGAGCTGGAGAATCCCACACAGCTGTGTTGTGGAAATGTTCTCAATTTCC | Dog | | | | | |
| 746 | cg17594003.2 | CGACAGAAGCGTCACGTTGGAAGCTTTTGAGTGATTATTTAATTAACCAT | Dog | | | | | |
| 747 | cg24651108 | GTGTGTTTTGGTGTTGTTTGTGTGTGCAATCTCGTGCCGCTTCCTGAACG | Dog | | | | | |
| 748 | cg13422693 | GGGCTGTGGGGGTCGGTCACCAGCCCCTCGTGAGAGCTCTCTGGTGTGCG | Dog | | | | | |
| 749 | cg23001363 | CGGGGCATGTGTTTACTGTTATGCAGATGAATGCAATATGGGAAGACATT | Dog | | | | | |
| 750 | cg11249940 | CGTTGCCATGGAAACTTCCTGTGCTGATAACAAGGATAATGAGGGAGGTG | Dog | | | | | |
| 751 | cg18361975 | CGCTGGCCATTTGTTGGCAAGTTCATATTTCACACTGCAGGGAATTGAAA | Dog | | | | | |
| 752 | cg24150369 | AGAAACCTTGGAACTTACCCATCAAGTCTCCGCTCATAACGTCCTTCTCG | Dog | | | | | |
| 753 | cg19723283 | CGGGCCAGTAGTCTGCAAAACTCATGGTAATTGTTTGGGTCTGATAAACT | Dog | | | | | |
| 754 | cg27220635 | GGCCATCCTGGGCTCCATGGTGAACGCCTTCATGGTGGGCTGCATGTTCG | Dog | | | | | |
| 755 | cg07697001 | CATTAGGGACAAGCTCATTAAAACCAGCCCTTGGGCAAAAGCAGTTTGCG | Dog | | | | | |
| 756 | cg10091639 | CGCACTTCTCAAAGACCAGAGCTAACAGAGGAAACAACGGGTGCCTAACG | Dog | | | | | |
| 757 | cg05917570 | CGAGTTATGGATTTATGGTCCCGGTCAAGCTCAGCCCATCCCCAGGCAGG | Dog | | | | | |
| 758 | cg06760134 | TCACACTCTGATGACCTCTTCTCCTCATTAAAACGTTCCCAAAGACAGCG | Dog | | | | | |
| 759 | cg16693246 | CGGAAGAGTAATTTGAATGAAATTACACTTGACAGCCTGTCTCCAAGCAA | Dog | | | | | |
| 760 | cg20473430 | CGGCAGGAGGTGGGGATGAATTAAAAACCATAAAATTGATGCTATCACTC | Dog | | | | | |
| 761 | cg15003065 | AAATGCACACACAAAGCGGAGCCCGGGTGAGAGGTGCCTTGTCAGAGGCG | Dog | | | | | |
| 762 | cg16565701 | CGCAAGCAGCATAAAATAGTGTTTAAACAAATATTGTATTGGCATGCTCA | Dog | | | | | |
| 763 | cg06627238 | TTCTCTTTCACAGTAGGCAGCAGAGCATATGGTTTTCTCTTAATGGAACG | Dog | | | | | |
| 764 | cg11464227 | GGTCCTGCATGTGGGTACGTACCAGCATTGTCCTCAGCTTCACAAAGTCG | Dog | | | | | |
| 755 | cg14765612 | CGAGAGTTCTGAGCAAGGAGATTTAACCTTTCAGCAAGGGGATGTGATTT | Dog | | | | | |
| 766 | cg08178288 | GATGCAGTAATTATTTTCGTATTTACGCCATTAACGATTGTAATTTAACG | Dog | | | | | |
| 767 | cg23954711 | CTTCAGTGTTGCCCTGGAAACTGTCCACCTTGTGATCAAAACTGTGGACG | Dog | | | | | |
| 768 | cg16314644 | CGGTGGTCGTGAATAAAAGAGAAACATTCATTTATGGTAATTGGTTTCAG | Dog | | | | | |
| 769 | cg05971481 | CTGATCATAAAATAACTCAATGGAAATGTGTAACTGCCTCAGGCAGCTCG | Dog | | | | | |
| 770 | cg19977633 | GTCACCTCTCATCACCATAATATTTACATCAAATGCAAAAGGCCTCCTCG | Dog | | | | | |
| 771 | cg22877608 | CGTGAATCATTCCATCACAATGTCCTTATTGATTTCCACAGGAAGAGGAT | Dog | | | | | |
| 772 | cg01334218 | TGATTTGTCAGGTCTGAAGCTTTGACGTGCCCAAGGGGCAGACCCCAGCG | Dog | | | | | |
| 773 | cg24862511 | TGAGGGATATTTACAAAGCTTGGATCCCGTGGTCCAACACTGACGAATCG | Dog | | | | | |
| 774 | cg14550267 | CGTGGTCCACCCAGGGATGTAAAACTGTGCTTACCGATGCATCCCATACG | Dog | | | | | |
| 775 | cg20429727 | TACTGCAATCCTTCCCACCTTGTTTTCAGTATTTCTTAATCATTAAGTCG | Dog | | | | | |
| 776 | cg24183320 | CGACTAAATAAAAGAATAATGTTTACTCAGAACTGATGTACTTGTTTAAA | Dog | | | | | |
| 777 | cg22391226 | GCTGGTTGTTGAAGACCTTGAGGCTGCAGCCGCTGGGGATCTTGCAGACG | Dog | | | | | |
| 778 | cg08471214 | CGCCTGAAATTCAAATTAGAGTTTTGCTGAAGCTGCTAAAACTCATCAAA | Dog | | | | | |
| 779 | cg24207053 | CTAATTATTGTGTTGTTTTCAGAGCAAATATGTCATATCCATGGCGACCG | Dog | | | | | |
| 780 | cg21603434 | AAGCCTTCTGATGCCATCGGTTCAATCACAGTTTTTACAATATCATCACG | Dog | | | | | |
| 781 | cg13219590 | CGCAGACAGTTTAGTTATGCAAATCTGATTGATCGAGAGCATATTGCAGG | Dog | | | | | |
| 782 | cg13667241 | TGCCGCTGTATTAATACACATCAGGGGCCAGCTCCTAGCACTAAATCACG | Dog | | | | | |
| 783 | cg09711459 | AGACAAAAAGATTTTGTCTCACTAAACCTGTTTATTGACCAAAACTGTCG | Dog | | | | | |
| 784 | cg15122264 | CGTTGGCAAGAGCGTCATCAGAGTCAGCTGGGAGTGACAGGCATGTTCTA | Dog | | | | | |
| 785 | cg10190711 | CGCTCTGTTAAAGTTTGAATTAATTTTTCCCATGGGTTTGTTCACATTCT | Dog | | | | | |
| 786 | cg06487902 | GGCCGGTGGGGCAGTCCTTGAGGAAGCCCTTGTACCACTCCTGCAGCTCG | Dog | | | | | |
| 787 | cg27594013 | CGACTGGGTGCAATGATAAATTATGAATTTTCTAATTTGCTGCATAATGA | Dog | | | | | |
| 788 | cg15613148 | GTTCTGAAGAAAACTGGTAAGCTGATCTCTTCATGTAATAACTTCTCCCG | Dog | | | | | |
| 789 | cg25836137 | GCAGTGTGAAATATGAACTTGCCAACAAATGGCCAGCGTGCTGTGCCTCG | Dog | | | | | |
| 790 | cg18952493 | GGGGGTGGAGAGCGGCTCCACGTCTTGATGACAATATGCCATACTTGACG | Dog | | | | | |
| 791 | cg16188689 | ATTACCGTCTTTGTTCATGTTGACTTCCAAACACTTCTTCAGCCGACACG | Dog | | | | | |
| 792 | cg08235698 | CGTCGTCAGTAAACTGTTGGCAGGATACTGCACCAGAGGTATACTTTTTA | Dog | | | | | |
| 793 | cg02886263 | CGCAGGGAAAATGAGGGACTTTTGGGGGCAGATGTGTTTCCATTCCACTA | Dog | | | | | |
| 794 | cg10122105 | GCGTCAAGGGGGCTCTGGAGAGCCATTTCCTCAAATGCCCCAAGCCCTCG | Dog | | | | | |
| 795 | cg19880080 | AAGAGGTGAATGCGACCGGCAAAGCCATTGCTCTCATGATGGCTGGCACG | Dog | | | | | |
| 796 | cg27276953 | CGCCACTATGTTGACAGGCTATTTGACCCTGATCCCCAGAAAGTTCTACA | Dog | | | | | |
| 797 | cg17860762 | CGCAGATTAAAATAAAATAGGGACTTTGCCGATGGATGGAAGTGGTAATT | Dog | | | | | |
| 798 | cg26739848 | TATGGTCATGAATTGTGGGCAGCACTACTGCCCCCTCACCTGATACACCG | Dog | | | | | |
| 799 | cg18741172 | CGAGACATGGTTGTCGAGAGCTGGCTCATGGTCCTGTTAAAATTGATGAA | Dog | | | | | |
| 800 | cg01938167 | AGTTATCAATAACATTTCCAAGGTTTGTTCCTTCATCCAACAGGCCAACG | Dog | | | | | |
| 801 | cg03568747 | GCGACCACGACGACAGATTGTCTATCCTCTGGAAAAGCTTGGCATAAACG | Dog | | | | | |
| 802 | cg19038076 | TGTTCAATCTACTGACATTAATGGCTACCTCATTAATCATAGCATTCTCG | Dog | | | | | |
| 803 | cg03266055 | CGCGAAGTGTGAACTCTCAACAGAAGGAAACATGAGACAACTGAAGTGCC | Dog | | | | | |
| 804 | cg12698769 | CGAGGGCACCATGTAGCACAGCTGGACCCCCTGGGGATTTTGGATGCTGA | Dog | | | | | |
| 805 | cg10040343 | CGACTGGCCGCCCTGAGCGCAATTCAATTCAACAGACATTTACTGAGTGC | Dog | | | | | |
| 806 | cg00724865 | TGTTTAACTTGCCTTTTTTACTTCTACAAATTATATTATGAAATCAGACG | Dog | | | | | |
| 807 | cg15801833 | CGGCAGCTACCTGGAGAATGGGAAAACTGATGAGTACAGTCTGTTGGGGA | Dog | | | | | |
| 808 | cg20813565 | CGCTTTTTTGGTGTGGTCTGTCTGGATAAAAGCGTGGCATCACCATAATC | Dog | | | | | |
| 809 | cg22058025 | CGACTCCTGCGAGCCCTTGTTCTGTGTGATAAGGCAATCTCCTCCGGGAT | Dog | | | | | |
| 810 | cg17421372 | CGGCTCCTGCTTCTGCTGCTGAGGTTCCTCCGCCGCCATCTTTAAACAGC | Dog | | | | | |
| 811 | cg16366997 | CGCTAATATGATGGATGTCAGTATCAACATTCAGAGTGATCTCGACAGGT | Dog | | | | | |
| 812 | cg26638661 | CAACACTGTGGCCCCAGCAATCGAAAAGTAATTCCAGCCCCATAGAAACG | Dog | | | | | |
| 813 | cg05268271 | CGAGGTCACCAAGTTGTGGTTACCAAGGCAACGTCACATCTAAGAATATG | Dog | | | | | |
| 814 | cg21739934 | GGCCTAATCAGTTAATACTGATAAACTGCCAGCAATAACTGCCAATTGCG | Dog | | | | | |
| 815 | cg01757168 | CGCTCTCTGAAAGGGCTCCGATAATCTGGAAGGCAGAGATAAGGAACACC | Dog | | | | | |
| 816 | cg05774801 | CGCCGGCCTGCTCCAGCACCTCCTTCATGGTCTCGTGGCCCAGCAGGTTG | Dog | | | | | |
| 817 | cg05046398 | CTTCTAGGCAGAGGTGGCATTTTGCTTTGGCTGTGACAACAGGCAAACCG | Dog | | | | | |
| 818 | cg14311946 | CACCCAACACTTTTGCTTTCTTCTCCATCTCTGAAGATCTGCTCAAGACG | Dog | | | | | |
| 819 | cg02512248 | CGGCTGGTGTTGATTCTTAATAAGTGACTTCCTAAAGCTGAGGCATGGGG | Dog | | | | | |
| 820 | cg06354898 | CGGTGCATGCCTGGCTTCTGGCCTCATACCCAGCTCTATTCTCTGCACAC | Dog | | | | | |
| 821 | cg23039301 | CAGGAACAGATTTTTAATTAAATAAATAGCTGGGTCGGCAGGCAGGTGCG | Dog | | | | | |
| 822 | cg21031345 | TCTGATTTCACGCTCACACATTAACGAGAGCTGACACTGGAGATTAGACG | Dog | | | | | |
| 823 | cg06337299 | AAACAGACGACTCCTTCTGGAGCCAGCTGGCGATGTAATGCAATTAGTCG | Dog | | | | | |
| 824 | cg21202493 | CGTGCCACTGATTATAAATAGTCCATCAGCTCTGCCATGTGTCATCAGCC | Dog | | | | | |
| 825 | cg13264155 | CGGGGAATGCAAATATTTATTTTTCGATTGTACTTTTGCAGGTGATTGCC | Dog | | | | | |
| 826 | cg00993368 | CGGCCACGCTTTATTAAATTCTCATAAACCTGTCAGGGGGGACAGGGCTC | Dog | | | | | |
| 827 | cg24206172 | AATTACTGTAACCATACTGAATAAAAAATACTGCCAAGAACAAAAATACG | Dog | | | | | |
| 828 | cg13279750 | CGGAGCCATCACATGATACAGGTCCCCCATCATGTGATGAGACAAATCTG | Dog | | | | | |
| 829 | cg04220619 | CGCTCTGTTTTTTGAGGAACCTGAATGAACCTGAAAGCAAAGACCGAGTG | Dog | | | | | |
| 830 | cg05347535 | CGCGGGTGGCTCTCTGCAGGTGTCAATTTATTCCAGAGATGAGGACACAG | Dog | | | | | |
| 831 | cg22762903 | CCCTGTTCTAACGACTCTCCTTATTACCTGACAATCTATCTGAACTATCG | Dog | | | | | |
| 832 | cg25831888 | CTTTAAGTGATCTGGCTGTGAACTGTTCAAATCTCTAATTACATTATACG | Dog | | | | | |
| 833 | cg19761677 | CTCCTGGCCCAGCTTGCTGGCTGCTGGGCCGGGAGGGGCTGCCAGGCTCG | Dog | | | | | |
| 834 | cg06774176 | CGGGGGCCAGGGCCTGACACAGACTTATTGGATTTCACGGTGTTTTGCTG | Dog | | | | | |
| 835 | cg09222634 | CCGAGATATTACATTGTTTCTGAGTGAAGCTAAAATCTCTCCCTGACACG | Dog | | | | | |
| 836 | cg12399476 | CGGTTGACGACACATGACAAACTCAGCTATATGGAAACAGAATCTAGGTT | Dog | | | | | |
| 837 | cg06282832 | TTTCAGAGCCAAGAAGATCAATGCGTTCATGCATCATGAATTAGGCTCCG | Dog | | | | | |
| 838 | cg24806294 | CGGACGGACGGACAGACCTCCTTCCTAAGCACAATAGCACCAGCTCCCCG | Dog | | | | | |
| 839 | cg00713411 | CTGTGGCATGTTCTGCCCGGTGGAAGGGTCCTCGGAGAACAAGACCATCG | Dog | | | | | |
| 840 | cg25670404 | CGTTCTGTTCCCACTAATGGCTTGGGGTAGGGCAGCCAGGTCTTACAAGG | Dog | | | | | |
| 841 | cg03990706 | CAGGATTTACAAGCAAGACAAGAATTTCTCATTAAGTGCACATTAACACG | Dog | | | | | |
| 842 | cg05357078 | CGGGGAAACCACATAAGCCAGAGAGACACTCGCATCTGAAACCAAAGAAA | Dog | | | | | |
| 843 | cg21538469 | TTTAAACAGGTGGTTTCCCTTTAGTGGAATCACTGAACTGGTAAATAACG | Dog | | | | | |
| 844 | cg18468479 | CGAGGGGTGGGCTGGTATCTCAGAAAGTGCCTGACACACTAACCAAGCTG | Dog | | | | | |
| 845 | cg03436411 | CGAGGAAACTTCATCTTGAAAATGAACTTAGATGAGATGGTGCCGGTGGA | Dog | | | | | |
| 846 | cg20747487 | TTAACTGTGAAATTTTATTTCCGTTAAAAAGCAAGCCTGTAATCAAAACG | Dog | | | | | |
| 847 | cg25928579.1 | CGGGGTCTCTAATGGATGCAAATGATCATGAAAAGACAGTGCAAAATATG | Dog | | | | | |
| 848 | cg27394581 | CCCTTGCTGGCCAGCCGGCTGAGGATGCTGCGCTCGGACATCTGAAGGCG | Dog | | | | | |
| 849 | cg25370017 | CGAGGTCAGAGAGACCCTCAGTGTTAGAGCCGTAAATCCTGACATTATAA | Dog | | | | | |
| 850 | cg27299890 | AGAGATGCATTTGTGTTGCCTCTGGGTACACTGAAAAGCCACTTAAACCG | Dog | | | | | |
| 851 | cg24566573 | CGAAACTGGGGAGACTTGGAAAACGCTGAGGCCAAACTTTCGATATTGAT | Dog | | | | | |
| 852 | cg03773588 | GCTGTCAGCCTGTGATGGATTCCTCACTTACTTCAATGAATGTTCTAACG | Dog | | | | | |
| 853 | cg02918319 | TTATTGGAGTCCCGAACAAAAGGTGTGGTAGGAAGGCGAGGTCCCCTGCG | Dog | | | | | |
| 854 | cg26504436 | CGCTGGTAGAAAATTCATTTTAGATTTGCTATTTATAAAGTACTCAGTAC | Dog | | | | | |
| 855 | cg24936113 | CTAGGAGATCAGGGTGACCTAGCCTTGCCCTGTAGCTTCCCTCTTAGACG | Dog | | | | | |
| 856 | cg25473687 | CGGAATGAATTTATTACTCTTGCCCATGTGAATCCCCAGTCATTCCCAGG | Dog | | | | | |
| 857 | cg14444871 | CGAGGATGCAAGCAGATCACTCGAAAAGCCTGCGAGCACTTCATCTCAGA | Dog | | | | | |
| 858 | cg02188837 | GAGCATGGGACAAATGACAGCATGGCCTGAATGCAGCCCAGACGTCATCG | Dog | | | | | |
| 859 | cg19226790 | TCATGATATTAAAGCCGTGTCAATTAATTTCTCCCCCTGCTTGATAAACG | Dog | | | | | |
| 850 | cg23585860 | CGCCAGCCTCGGCGCCTGGACTCGAGCCATTGTCGCCTTCTAGGGACCCC | Dog | | | | | |
| 861 | cg21067017 | CGCGCTCATCACCATGGCCATCCTGCAGAGCCCGCAGAAGAAGCTGACCC | Dog | | | | | |
| 862 | cg05709691 | CGGTTCCACGTCAGAAAGTGACACGTCAACTTTCCACGGCTTTGATGAGG | Dog | | | | | |
| 863 | cg14805279 | CGGCTGGCTATGACAGCAGGCACTTAATTTGCATGTTCGACAGACAACAA | Dog | | | | | |
| 864 | cg27461453 | CGTGGTGCTTTGATTAATGATGCTGGTGGGACTCCCAGAAGACCAAAGAG | Dog | | | | | |
| 865 | cg22283959 | CGATGGGGGTTTTACCTACCAAACGGACGAAAAACAATTGCTCTATGACT | Dog | | | | | |
| 866 | cg23779125 | CGCCTCCTTGTGCTTGGACGAGAACTGGAAGGTGCCCTTGTCCTTGTCCA | Dog | | | | | |
| 867 | cg21056360 | AAATTTTGATGGTGCCCTGCTTCTAATATACATTGCAGTAATTAGATTCG | Dog | | | | | |
| 868 | cg17803853 | CGGAGACTAACCATAAATCATGTCTTTGAATCACTGACAAAGATAAAGGA | Dog | | | | | |
| 869 | cg22253511 | CGAACTGCTCCACAGTGGCAGCCTCCTGTGTGCTTCACCAGGCCCTGGTA | Dog | | | | | |
| 870 | cg24763772 | ATATATCACAGTGTTTCCACATCAAAACTGATGGCAAAGTTACATGTCCG | Dog | | | | | |
| 871 | cg05032451 | TCTGCTGGGTCCGAAGATCCCTGATGCCAGATGCTTGATCTCTGATGGCG | Dog | | | | | |
| 872 | cg27146637 | AATGTGTTTCCTGTTGGTCTAGCTCGAAAAACTCTTTGCTTCCTCCTGCG | Dog | | | | | |
| 873 | cg07425396 | TCCCAGGAAACGAGGCCGTCCTCATTGAGGTCATGCCCCTTCCACTGTCG | Dog | | | | | |
| 874 | cg06892949 | GGGGACCAGGCATCAGATTGGAGTCAAGATTCCGAGAGTGTCATTTGCCG | Dog | | | | | |
| 875 | cg20769452 | CGGAATGCCATTTAATTTTTCCGAATGCAGTCCGTCATTAGGAATCTTAG | Dog | | | | | |
| 876 | cg22464003 | CGGGGATCCAAAGGAAAGTTGGAAGCCATCATCACACCACCCCCAGCCAA | Dog | | | | | |
| 877 | cg15869291 | AAATATCAGTTCCCATACTAAATTTTTATCCCTGGCTGATAAATATGACG | Dog | | | | | |
| 878 | cg15079402 | TCTCAGCATCACATCTTCTGTGCTCTTTATTTTTGCCGGACCAGTTTGCG | Dog | | | | | |
| 879 | cg09771641 | CCTGTTACAAACTGGTTCTGTCACCACAGAGACTTCAACTGATCACCTCG | Dog | | | | | |
| 880 | cg02498174 | TGGCCCCATTCCCAGAATTTAACAGTAGGGGCCTTGTGAAATGCTAAACG | Dog | | | | | |
| 881 | cg22806872 | CGCTGGTTGCACTCATGGCTGCATGACTCCTCCTGCATCCCAGCAGCACC | Dog | | | | | |
| 882 | cg06624763 | CTGTCAATCCAACACTTTTAGGGTAATTTAACTTAATAATACCTTTGTCG | Dog | | | | | |
| 883 | Cg03596170 | CATCAATTACTCTTCCACACAAAACAGAAACCTTGTAAAATTTATTTTCG | Dog | | | | | |
| 884 | cg13003121 | AGCAATGGCCCTTCTGTGGTCAGAATGGCACATTCAGCAGCAACTATTCG | Dog | | | | | |
| 885 | cg22763835 | AAAATATCAATGATATCATTCTGAATTTTCTGTTCATCACTATCCAAACG | Dog | | | | | |
| 886 | cg17162734 | CGCCCTGGGGGGTGGCATATGGCGCCCTGTCACCTACCCAAGAAAAAGAA | Dog | | | | | |
| 887 | cg03635671 | GCCATGTGGGAAAGACATTAGTGCCGGCCGCCCTGCCAGCCAGGAATGCG | Dog | | | | | |
| 888 | cg06626742 | AACAATACAGGTTCAACAGGAGTTCTCATCGGTAGTTCATTTTTCAAACG | Dog | | | | | |
| 889 | cg26625726 | TCAAATGATTTTGCAAGCCGAATCTGGACTGTGGATGATTATCGATTACG | Dog | | | | | |
| 890 | cg16593601 | AACGGGGGATTAACAGGTATTTGTCTCAGCTAATTTCACTCTCTATACCG | Dog | | | | | |
| 891 | cg02684102 | CGGTTCCTGTCCTCTGTGATAGTGAGTTTTAAAAGGGAAATCTCCAAAGA | Dog | | | | | |
| 892 | cg05039637 | CGGGCTGTTGGGTAGGTTAACACCCTTCAAGGCCTCTTTCATGTCCAATA | Dog | | | | | |
| 893 | cg26166546 | CGTCCTTTCGTGTGCAGAAACCATCGATTATTATGGGGAAATCTGTGACA | Dog | | | | | |
| 894 | cg07588875 | CGCCTCCTTCCTGTGGGAACCATCCCCCGCTGGAGTCATCTCTGGTCATA | Dog | | | | | |
| 895 | cg07254551 | ACAGGGGCCCCGCTGGGTCAGAGTGAGGAGCTGCAGCCACTGAGCCAGCG | Dog | | | | | |
| 896 | cg06283343 | AATGTGACTGCATGTTCGAGATCACTGCCTCCCATCTGCAATTTGTGACG | Dog | | | | | |
| 897 | cg27066705 | CGAAAGTTTGGCCTCAGCGTTTTCCAAGTCTCCCCAGTTTCGATTGTTCT | Dog | | | | | |
| 898 | cg19518854 | TGACTATTTGCATCATTGCTACACCTGTAAAATGGATAAGCAAGCATACG | Dog | | | | | |
| 899 | cg25365217 | ACTTATGATGACAGTCTATTTATTTTGCTATAAACCCTCAGCACGTAACG | Dog | | | | | |
| 900 | cg06166628 | CGACTGCATCCTGGCTGATATTTGATAATAATTTTAACAGATAAATGTAG | Dog | | | | | |
| 901 | cg21074294 | CGTGGGCACATAATTGATCCATTTGGAGAAAAAAAATGACAGTGCTCTAA | Dog | | | | | |
| 902 | cg10727440 | CGGCTGACCACCGCTGACTTCTGTGAATATCCCTTTCAGGCTGAGGACCG | Dog | | | | | |
| 903 | cg11067179 | CGGCAATCTGGCACTCATCTGTGTCCACACAGCGGTGCGGATCATCCTCC | Dog | | | | | |
| 904 | cg09922950 | CGCTCTGGGAGAGGAGAAAGTTGGCAGAGAGAAGCCTAGCAAATATATAA | Dog | | | | | |
| 905 | cg07397105 | CGCTGCAGCACAGAGATTTTCTCTGAAGCCAGCTGGGAGCAGGTGGATAA | Dog | | | | | |
| 906 | cg03761238 | CGGGAAAATGCTGCCGAATTCATTTAGCTGCTGAGTGTTTTGATCTGGGC | Dog | | | | | |
| 907 | cg18870419 | CGTGGCATCTCAATCAGCTGTGTTTAGATGCTGCTGTGACACAAGATTAT | Dog | | | | | |
| 908 | cg15713442 | GAGCCTGTGGGACACCAGCCGGGCCGCTTTATCTTCTTCATTAAAGTACG | Dog | | | | | |
| 909 | cg14091483 | GTCATGGCTGTTAGTGGGTACTCTCCATAGGTGGTCTCTAGGACTGGCCG | Dog | | | | | |
| 910 | cg27508620 | CAGGTCAGTTTTGTTACAGAGGCCAATTAGCAGTGCTACCTTCGCCTGCG | Dog | | | | | |
| 911 | cg17296202 | ACCAAACCATGTAAATTTACTTTGTTCTCAGCTATGTTTCCAATTTCACG | Dog | | | | | |
| 912 | cg18623536 | TTTTATCAGCAGCAAGCAGGCCTGATTACTGACAATTACACTCTGATCCG | Dog | | | | | |
| 913 | cg22763788 | CGGAGGAATTAAGCTGTAAAAATTACCTGCAGTAATTTGGAAACATTTAG | Dog | | | | | |
| 914 | cg17881605 | CGGGGGTTCATAATTTTCTGAGGTTGTTTTGATAGACTTATTGTGCGGAC | Dog | | | | | |
| 915 | cg00267482 | CGGCAACTTCCTGCTGTTTGGCGAGTACACAGTGCAATGCAGTATGTCTG | Dog | | | | | |
| 916 | cg11972313 | CGCCGTATTTTTTGTCCTGCAAATGAAATTCATTGCTGCCGGTTAGTGAT | Dog | | | | | |
| 917 | cg13756118 | TCTCACCTCTAGAGGTGGTCCCTCCAGGTCAGGGTTGAGGCACATGGACG | Dog | | | | | |
| 918 | cg20092344 | CGCTGGCTTGTGCCCAGGACTTTACCCCGCTTGGAACCAAAGGAGTGTTC | Dog | | | | | |
| 919 | cg06587088 | AAACAGGGCCTGCGCGCCTTAATTACATCTGATTTTTAATTCAGAATACG | Dog | | | | | |
| 920 | cg13679010 | GCCTCTTGTTCCCCGTGTAATTTGCAGCCAATCATCAGTAGGTGTCCACG | Dog | | | | | |
| 921 | cg25019647 | GTACTGTAGTTTCTCATGGAAACACTTTGTGAAAAGCCAGTCGTATATCG | Dog | | | | | |
| 922 | cg13888366 | CGGGGGCTGGTTTATCAAATGTAGCAAAGTGCAGGTTGAGAAAGTCAGAA | Dog | | | | | |
| 923 | cg19272215 | GTTGACGTGTCACTTTCTGACGTGGAACCGAATAAGTCCTCCGTGGTACG | Dog | | | | | |
| 924 | cg25731101 | CGGTTAAGCAAATTATGAGGAGGGGGGCGATGTCCTCACTCTAATAATTT | Dog | | | | | |
| 925 | cg10325143 | CGCACACATACCCGACCAAGGCCTCAACTCTGAGCAAGAGCTAGGAAGGA | Dog | | | | | |
| 926 | cg12376071 | TCCTCTGCCAAATCATCCAATAATTAAGTTTTATGTTATTTGTATGAACG | Dog | | | | | |
| 927 | cg12935694 | TGGTGGGTGAAAATCTATCCTGGATAACTCCTGGACCAGTACCAATTCCG | Dog | | | | | |
| 928 | cg20962788 | CGGTCATGTGTGTATTTTTTCATAGTCCCACCTTGGAGCATTTATGTAGA | Dog | | | | | |
| 929 | cg10560122 | CGCTGCCGCATCCTGACTGCTCCTGGCAACGGGGTCCCGCAGCCAGGCAG | Dog | | | | | |
| 930 | cg15660755 | CGGTCCCTTGTGGTCAGCCCTCATGAAGACATGAGAACCTGGCTCAAGTA | Dog | | | | | |
| 931 | cg26490921 | CGCAGCACGCCAGCCTAAGCTGAGAGCTGTAATGTCATTAAATTGCAAAT | Dog | | | | | |
| 932 | cg19355530 | CACGTCTTTGGCGGCGCTGATGCCATCCTCTTTGAGGTTTTTCTCAATCG | Dog | | | | | |
| 933 | cg25467381 | TGTGGGATGTTCAGAAAGTTGCCGTCCCGGGGTCCGATGCTGACAAACCG | Dog | | | | | |
| 934 | cg24025580 | CGACTCTACCTCTCTGAAAATTGGATCTGCTTCTACAGCAACATCTTCCG | Dog | | | | | |
| 935 | cg05094019 | CATTTCTGTTCTTCCTTGCAAGATTGTGAATGCTTCGGCCACTCCAATCG | Dog | | | | | |
| 936 | cg18643010 | TGATTCTGAATGAAAACAAATTGGCCCGCTTTCTTCTCCAAACAGACGCG | Dog | | | | | |
| 937 | cg19268153 | AAATTCAGTTTTGAGAGTAAACTCTCCGCATTTTCCACCAAAACATCCCG | Dog | | | | | |
| 938 | cg22671203 | CGTCTTGCTTGCCAATTGCCATAGAAATCTTAACACACCATGAAGATTGT | Dog | | | | | |
| 939 | cg19542597 | ACCCACGTCATGACCACAGGTCTCAATATAAAATTATAGAGAACACAGCG | Dog | | | | | |
| 940 | cg25986664 | CGATAGTCTTGACCGAGAATGCTGCCTTGACGTTCCTTTGGTATTTTTAC | Dog | | | | | |
| 941 | cg09566653 | TCCTTGAACTGCTTCATTCGAGCCTCCAGAGGGACAATGGCCCTTTCTCG | Dog | | | | | |
| 942 | cg24354672 | CTGCAATAGGCCGAGCCGGCTGCATTAAGCTTCTTGCAATCAATAAAGCG | Dog | | | | | |
| 943 | cg23618499 | ATTACAGGAAATTATTGTCATTCTCCTGTTAAAATAACACATAATCATCG | Dog | | | | | |
| 944 | cg16021411 | CGGGTAAAAATGGGGCTGTCATCTTGAGCTACAAGTTAGAAAGTTATTAC | Dog | | | | | |
| 945 | cg24477176 | TGGTAGGAGCGCTTGCCTCGTCGCTACAGCTTTTATAGGGCTGTAAAACG | Dog | | | | | |
| 946 | cg21508496 | CGCTTCGGGAAACCAGCCAAGCGCAAAATGTGGAAACTAAAATCCTTTGG | Dog | | | | | |
| 947 | cg03123810 | CGCTGACACCCAGGAAGAAAAATTGGACCCCTTGGGAGATGGACCGATTT | Dog | | | | | |
| 948 | cg23825683 | CTCCTAACAATCCTCCAGGTGTGGTTTCAGAACCGAAGAGCCAAGTTCCG | Dog | | | | | |
| 949 | cg04294426 | CGTGGCAGGGCCGAGGTGAAGTTCTGACTCTGCATTTCTCTAATCCCCAG | Dog | | | | | |
| 950 | cg24276148 | CGAGGGATCTCAGCCCCACAATATTTCACTTTAAAGAGAAATGGAAACAA | Dog | | | | | |
| 951 | cg05170661 | CGGCACTTTCTGATGTAACAATCGAGTGTGTGTTTTCCCAACTGCAGCTT | Dog | | | | | |
| 952 | cg16446343 | CGAGGCCATAATTAATTTGAGATTTATTTTTATTGGAGAACTCGAGTCTC | Dog | | | | | |
| 953 | cg13531810 | CCTTGTTTCAGTCTTTACCACTGGCATCTCCGTTGAGGCTGCCTGAAACG | Dog | | | | | |
| 954 | cg11137703 | CCTGGATGTGGTGCTGCTAATGGGTGCTGCACTGGGCCAGATGGGCATCG | Dog | | | | | |
| 955 | cg26418994 | CGACTCGCCAAAAGTTCTGAAGGCATCGTGTTGATTAATGCAATTTATCT | Dog | | | | | |
| 956 | cg08587914 | CGGGCTTATCTGTAAACTGACTGAATTCACACTGGGATGCTAAGAAAATA | Dog | | | | | |
| 957 | cg04931708 | CGCAGAGTTCTTGGCGCACCAATGACTCGGGGACAATCACTACTTATTTC | | Cat | Mouse | Rat | Human | |
| 958 | cg05317739 | CAGCATTGAAGAGCGATTTCCTTAATGAGATGGAGCGGTTGCACGTCACG | | Cat | Mouse | Rat | Human | |
| 959 | cg14104121 | TGAAGCTGTTGTCTGGAGGCAATGATCCCAAATCTGAAGGCTTTATTCCG | | Cat | Mouse | Rat | Human | |
| 960 | cg26391562 | CGCCAGAGAAAGGGGCTTTTCTTGTTGCTGACGACATGTGTGTGACATGT | | Cat | Mouse | Rat | Human | |
| 961 | cg18513739 | CCGCCTTGGCAGCAGAAGGTGACATCTGGTGACTAATGGCCGCGGCTGCG | | Cat | Mouse | Rat | Human | |
| 962 | cg24352905 | GACCCCTTTACTCCGTGACGTGCAACCGCTCCATCTCATTAAGGAAATCG | | Cat | Mouse | | Human | Universal |
| 963 | cg01061575 | CGCAGGTTGAAGTTCATGAGTTCGCCCAGCCTCAGTGACCTGGGCAAGAG | | Cat | Mouse | | Human | Universal |
| 964 | cg02553441 | CGGAATTCAGAATAGTTATCACATTTAAAGCTGCTAAACCAATGGCAGTG | | Cat | Mouse | | Human | Universal |
| 965 | cg01409964 | GCAGCTTGCAATGCTCGAACAGAAATAAATTTCACTTCCTCAAACTCTCG | | Cat | Mouse | | Human | Universal |
| 966 | cg10491556 | GGTCAGGGTGTAGGGGTACCACCAGGCTGGGGAGCGCTCCAGGTAGTGCG | | Cat | Mouse | | Human | Universal |
| 967 | cg19908562 | CGGCGCTGATGCCATCCTCTTTGAGGTTTTTCTCAATCGCCTTGCTCCGC | | Cat | Mouse | | Human | |
| 968 | cg24860713 | TGGCTTCCTCGTGCTCCAGCGAGTGGAAGTGCAGGTGGTTGGGCAGCGCG | | Cat | Mouse | | Human | |
| 969 | cg12135913 | CGCTAGAAGGGAAACAGTCTCTGCATTCACAGGGCTGGGCTGTCAAGTGC | | Cat | Mouse | | Human | |
| 970 | cg02317025 | AGATGGGCCACTTTCTTCTGGCCACAAAGGGGCCGGAATGGAGCGCTCCG | | Cat | Mouse | | Human | |
| 971 | cg07566172 | GTCCTCAGTGACTTCATGAGCCCCCTGTTTACCTTACATGGTCACACGCG | | Cat | Mouse | | Human | |
| 972 | cg05262155 | GAGTTCGGGGCTCTGCTGCAATGTCTGCGAGGCTGACTTTGAGCGCCTCG | | Cat | Mouse | | Human | |
| 973 | cg09258317 | ATACTCTGGCTTGGGGACTGGAATAAAACACACGCGCTCTGAGGCAGCCG | | Cat | Mouse | | Human | |
| 974 | cg26880735 | TTCTGAATTCACCAAAGAGAGTTTTCTGTTATATTTCAGAAGTACTTACG | | Cat | Mouse | | Human | |
| 975 | cg20275558 | CGCCAACTCATCCTGCATCAAGTGCCCATTGAGTAGCACTGTCACTTTAA | | Cat | Mouse | | Human | |
| 976 | cg15182157 | CGAAGGGCAGTTCGTAAACAGCACTTGTTCTTTTAATAAAAGAATGTTTT | | Cat | | Rat | Human | Universal |
| 977 | cg11728741 | CGGCCAAACAGCTGAAGAAAATCAAAGACATCGAGGCGCAGGCCCTCCAG | | Cat | | Rat | Human | Universal |
| 978 | cg07056567 | GAGCTGGAGCAACTGAGACAGGAGGCCGAGCAGCTCCGGAACCAGATCCG | | Cat | | Rat | Human | Universal |
| 979 | cg08510667 | CGCCGGGATGAGCCGCCCCCTGGACACCAGCCTGCGCCTCAAGACCTTCA | | Cat | | Rat | Human | Universal |
| 980 | cg27208811 | CGAGCAGTATTGCTTATCAGAACACACCAACAGTCTTTTTGTAATATTCG | | Cat | | Rat | Human | Universal |
| 981 | cg24504442 | CTAGGTAAGTGTTGGAGATTTAAACCTACCCTTTGCGCCATCAGTCTGCG | | Cat | | Rat | Human | Universal |
| 982 | cg01245471 | CGGATTTCTATGAAGAAATCGAGGTGAGCTGCACCCCGGACTGCGCCACC | | Cat | | Rat | Human | |
| 983 | cg21801378 | CGGGCTAAACCCCGGTCCCGCCGTACCCATGAAGGACCACGACGCCATCA | | Cat | | Rat | Human | |
| 984 | cg00958552 | TGAACCTGGTATCTGGTGATCCTATCTTGTTTCATCTGTTCAAATTTGCG | | Cat | | Rat | Human | |
| 985 | cg03603326 | GGGTGTGTTTCTTCGGTCATTTTCTTGTTCACCTAGACGCCTGTCCCGCG | | Cat | | Rat | Human | |
| 986 | cg03740978 | CGCAGCACGCGGCACAGCGCCCCCTTGTCCATGGTCTCGAAGTCCGGGCT | | Cat | | Rat | Human | |
| 987 | cg04875128 | CGCCACGTACCCGCAGCAGAACCGCTCGCTGTCGTCGCAGAGCTACAGCC | | Cat | | Rat | Human | |
| 988 | cg13690286 | CCAAACCGGTCTTGCAGGTCCCAGGAAGGTGGCGTCAGCATCTGCAGCCG | | Cat | | Rat | Human | |
| 989 | cg05413840 | CGTCTGGCCGCATCTCAGATGTGTCACCTATGCCGGGAGGGAATAAGGAA | | Cat | | Rat | Human | |
| 990 | cg05542681 | CAATGACGAGGCAGGGGGGCAGGTGCTGCGAGAAATACTTGAAGAGCTCG | | Cat | | Rat | Human | |
| 991 | cg05968022 | ACATTGGCTGGAAAGTGGAGCAGCTCGTCAGGGACAATCAATGGCGATCG | | Cat | | Rat | Human | |
| 992 | cg06332339 | CAGACAGCTGTGGGACGGTGCTCGGAAGCGCCGTGTCACCGTAAACACCG | | Cat | | Rat | Human | |
| 993 | cg08069459 | CCTGGCGGCAATCAATTTGGGTGTCACATGAAATAGGCATAATTATTCCG | | Cat | | Rat | Human | |
| 994 | cg20435403 | CGCTAGCTAGTAGGAGGGCTTTATGGAGCAGAAAAACGACAAAGCGAGAA | | Cat | | Rat | Human | |
| 995 | cg11369802 | CGATGAGCTTCTTGGCATATTTCCTGACAATGTTATCTTCTCCGAAGATG | | Cat | | Rat | Human | |
| 996 | cg12448957 | GCACGATGATGGGGCAGGTGATACTGTAGGTCATGACCACCGTGAAGACG | | Cat | | Rat | Human | |
| 997 | cg12505751 | CGCCTATGACTACGATGAACTTCAGAGCAGAGTGTCCAATCTTGAAGAAA | | Cat | | Rat | Human | |
| 998 | cg12839840 | CGCCATTGATTGTCCCTGACGAGCTGCTCCACTTTCCAGCCAATGTCAGG | | Cat | | Rat | Human | |
| 999 | cg13447705 | AGTGGGGCTGGGCAATGTCTGGAGGTTTCCCTACCTGGCCTTCCAGAACG | | Cat | | Rat | Human | |
| 1000 | cg23449274 | GCGGAGCGTGTTGCCGGCAGTGCTGCTCTGGACTGTACTGAAGGCAGACG | | Cat | | Rat | Human | |
| 1001 | cg15993931 | TTCATGAGCCCCCTGTTTACCTTACATGGTCACACGCGGCCTAATGGACG | | Cat | | Rat | Human | |
| 1002 | cg16455800 | CGAACTGGAAAGCAGACTCCATAACATGCTAGTCAGTGGAGAAATCAAAG | | Cat | | Rat | Human | |
| 1003 | cg17115006 | TCCATGATCAATCCCATCATCTATGCCTTCCGCAACCAGGAGATCCAGCG | | Cat | | Rat | Human | |
| 1004 | cg18200760 | CGTCCTCTTCCGAAATGCAAAAGATCAGTGACTTTGACAGGTAGATGAGG | | Cat | | Rat | Human | |
| 1005 | cg18910772 | CTCATTTTAGACAAACTGCGAGTGGCATTTTGTTTATATGGTTCATTTCG | | Cat | | Rat | Human | |
| 1006 | cg19397128 | ATTACGTCAGTTTCAAGACACCAACACTATTAATATCAAAGGAGCCTTCG | | Cat | | Rat | Human | |
| 1007 | cg21773992 | CGCCGGTGAATGGTCAGTGCTGGAGTTTGAACAGGGCCCTGAACCATCTC | | Cat | | Rat | Human | |
| 1008 | cg24897861 | CGTGGAAAGATTATTCTGACTAACGAGGTTCTGGGCCACTAATTCATTAC | | Cat | | Rat | Human | |
| 1009 | cg24266105 | GCTCTGGAACAGAGCATTGAGTTCATTAGGGACTCTCTGATTTCCATTCG | | Cat | | Rat | Human | |
| 1010 | cg27287578 | ATAGAGCTTTCACTGAGCTTGTGCTATGACCTAAGCCTTCACGCAGAACG | | Cat | | Rat | Human | |
| 1011 | cg25477526 | GGACGAGAGAGACTGCAGCAAGGGTTTCACGTGATCGTGTGTAATGTTCG | | Cat | | Rat | Human | |
| 1012 | cg26390542 | CGCCTGCCACTATACACAAACGTTAAAATAGGAAAACGGAAAACACTGAC | | Cat | | Rat | Human | |
| 1013 | cg05455896 | CGCTTAATAAACATAATGATGACATAAGATGTAATTCTAGATGAATTTTC | | Cat | | Rat | Human | |
| 1014 | cg05129035 | CGCGTGTCTGTTGTCTGAGCAGGCAGCTCTCAGCATCACCCATCCTGCTG | | Cat | | Rat | Human | |
| 1015 | cg05090186 | TTAGAACTTGTAGATTTGAGCTGCCTGAATTGGCCAGACAGCTGTAATCG | | Cat | | Rat | Human | |
| 1016 | cg05675373 | AGGCGGGTTCCCGGTAGGGTGCGCAGGGTGCTGCGGTAGGTCTCATGTCG | | Cat | | | Human | Universal |
| 1017 | cg00310215 | CGCTGGCCCGGTGCCAATCGCCTTTCAAAGAGCCCCTCTATGATTAATCG | | Cat | | | Human | Universal |
| 1018 | cg06461219 | GGTGGGGAGAGCCCCCGGCAGTTGGAGACCAAAAACAATTTGCCCAATCG | | Cat | | | Human | Universal |
| 1019 | cg06997801 | CGGGGGCCAAGTCCAGGGCAGCCCACAGTCAGCCATTCAGGAAAGCCATC | | Cat | | | Human | Universal |
| 1020 | cg17437489 | GCTTTCTTCAAGCAAGTGCGTATTAATTTCTGTCATCCCAAACATCATCG | | Cat | | | Human | Universal |
| 1021 | cg00975350 | GGGCGCCTGCCAGGAACACTCAGCTCATAATAACCTCGCGGAAAACACCG | | Cat | | | Human | Universal |
| 1022 | cg02073955 | CACTTCTGAGCAGGGTGCAGAACAGTAAACTATAATTTAAACAAGAATCG | | Cat | | | Human | Universal |
| 1023 | cg02532525 | CTGTTACTGAGGGCGGCCCGGGCAGTAAGCAGTCTAGAGCCAAGGTGCCG | | Cat | | | Human | Universal |
| 1024 | cg06571003 | CGCGCTCCCATATAATTTATGACTGCCTCTGGTCCTGATGACAGATTATT | | Cat | | | Human | Universal |
| 1025 | cg07493901 | CGGGGCCAGCCTGGAAGATTGTCACTGTAACCTCTTCTGCCTGGTGAGTG | | Cat | | | Human | Universal |
| 1026 | cg08912313 | CGCCTTATTGAACAATTCATGGTTTACTGACCATGTTAATGATGTAATCT | | Cat | | | Human | Universal |
| 1027 | cg14334315 | TAAATAAATTCTGAAGTAATCACGTCTTTGCTCTCCTCCTCACGCACACG | | Cat | | | Human | Universal |
| 1028 | cg18502834 | CGCCGAGAAGCTGCCGGTGACCATGAGCAGTTTGCTAAACCAACTGCCTG | | Cat | | | Human | Universal |
| 1029 | cg19561061 | CGCACAGTATTTGCCGAATCTATGGAAATCTTGGAAATGCTTTGACAGCT | | Cat | | | Human | Universal |
| 1030 | cg21513836 | CGGGTCTGAAAGCACGTGGCTACGTCATGGAAAGCCAGTCTTTTCAATGC | | Cat | | | Human | Universal |
| 1031 | cg22673296 | CTGCCCCAAGATTGATCTGGTCCATAGGATGCCCCAGATCATTGCATTCG | | Cat | | | Human | Universal |
| 1032 | cg26869371 | TGGCAGGATCCATGTCAGTATCCATTTGTGCATTATAGTGTAGTGCTCCG | | Cat | | | Human | Universal |
| 1033 | cg25430489 | CGGAGGCGCAAGTCCTGCCAGGCCTGCCGCTTCATGAAATGCCTCAAAGT | | Cat | | | Human | Universal |
| 1034 | cg19912994 | TGGCCAAGGGCGAGGCGTCGGAGAAGATCATCATCAACGTGGGGGGCACG | | Cat | | | Human | Universal |
| 1035 | cg02322553 | CGTCACTGTGCGTGCCAACATTGCTGCCATCACTGAATCAGACAAGTTCT | | Cat | | | Human | Universal |
| 1036 | cg01053290 | CGAGGGCCTGACACACTAATTACAGTGTGAGCACTCAATCATCAACACCT | | Cat | | | Human | Universal |
| 1037 | cg13058338 | CGCGGGCTGGTTATGCATTTAAATGTTATTTAATTGGATTGCGGAGGAGC | | Cat | | | Human | Universal |
| 1038 | cg06796779 | CGGCTTCCCACCCACGCCACCCAAAGAAGTGTCTCCTGACCCTAGCACCA | | Cat | | | Human | Universal |
| 1039 | cg21084050 | CGCTACACCCGCCTCCGGGACTGGCACCACCAGCGCTCTGCCCACGTGCT | | Cat | | | Human | Universal |
| 1040 | cg22569774 | ACCTTCCACTTTGGTAAGGGTCAGGACCGGACTGTTGCAGGCGCTGAGCG | | Cat | | | Human | |
| 1041 | cg00060304 | CGCATTGAGCCCCTTAGTGCCTTGATTAACAGGCAGATTAACTCTTACTG | | Cat | | | Human | |
| 1042 | cg13692433 | GAGTTGCGAGTGTGCTGAGGCTGGGACTGTCACTCATTCTCCGATCAGCG | | Cat | | | Human | |
| 1043 | cg01079397.2 | TGGCTACGGCTCCTCTTCCGTCTCCTCAGTGCGGGGAACATGTAGAGCCG | | Cat | | | Human | |
| 1044 | cg15273822 | GAATTCTCTACACACCAGTAACGGGATTTCAATTCCGATGGACTCTGCCG | | Cat | | | Human | |
| 1045 | cg25607172 | CAGGTGAAAATCTGGTTCCAGAATCGCAGGTACAAGTGCAAGAGACAGCG | | Cat | | | Human | |
| 1046 | cg15334728 | CGCTGCCTGGACTCAGAGCACAATTGCGTTTCCTGCGGGTTATTTTTGGC | | Cat | | | Human | |
| 1047 | cg20655169 | CTTAGTAGCCCTGTGCCTGACCTTCGGGGTGGAGGTGGGCTTTAAGTTCG | | Cat | | | Human | |
| 1048 | cg14193568 | TGCAGAAGCAGCAGAGCAGGACGATGAGACCCCAGATGAGCCAGAAGCCG | | Cat | | | Human | |
| 1049 | cg08695595 | CGCTCCCTCAGCTGAGGGCTCCGCTCCACAACAAGATTTACTTTAAGACA | | Cat | | | Human | |
| 1050 | cg00130134 | GACATTGAACCCAAATTTGATAGACTGCTGTTTTTCTGGTCTGACCGTCG | | Cat | | | Human | |
| 1051 | cg00190912 | CTACAGGTAATGGTCACTGATGCTGGTGGGAAAATACTCCTGTTGGAGCG | | Cat | | | Human | |
| 1052 | cg08916715 | CGGCACTGCCAGTCAGGTTAATCATCCCTACTTTAATTAGCTGTTCGGCT | | Cat | | | Human | |
| 1053 | cg09829700 | CTCGTTGATGGGCTGGTTGTACAGCCGCAGGATCTCCTCCAGGCTCAGCG | | Cat | | | Human | |
| 1054 | cg01378910 | TGGCTTTTCAGTACATTAAACAAAGTTTCATAAATGTCTCCGCTCTCTCG | | Cat | | | Human | |
| 1055 | cg01499097 | CGGCAGCCTGCCTGAGATCCTCTAGCACTGAAATAATATCATCCAGCACG | | Cat | | | Human | |
| 1056 | cg01935380 | TCCAACTGAGATAATAAAGCAAACAATAACATTTGATCATGATGTCTACG | | Cat | | | Human | |
| 1057 | cg01996115 | GGTAAAACAGACCTGTCTGAGGGTCTAAAGTGTCCATCTCTTCAACCTCG | | Cat | | | Human | |
| 1058 | cg02017534 | AATGTTCAATAATACATTCTCCGCCAGGCTCAATTCATCAGGGAGTTTCG | | Cat | | | Human | |
| 1059 | cg02385429 | GCTGGCGGCGCTGGAGGGATGGTCGGGGGCTGGCTGGCTGCAGGCTGGCG | | Cat | | | Human | |
| 1060 | cg02582218 | GGTACTGTCTTTTGTGACAAAGTTTCCTCTGGTAAGTTACAATTATAACG | | Cat | | | Human | |
| 1061 | cg04084271 | GCAAAATAATGTAAAAATAATGAGCTCCTATTCCCCTTTGATGTGCAGCG | | Cat | | | Human | |
| 1062 | cg04110765 | CGGGGCTATTTTCTGAGAGTCTCCTGACACCATATCTAATTTCCAAAATG | | Cat | | | Human | |
| 1063 | cg04371354 | AGAGCTATTTTAGAAAACCCAAATTGCCAACAGTAATAATCTCTCCGCCG | | Cat | | | Human | |
| 1064 | cg04735310 | TCAACACGGTGAAGTTTTATTGTATTTCCGGTCCCCAAGATTTATACCCG | | Cat | | | Human | |
| 1065 | cg04895159 | AGATACAGGTATCAAGGCCAAATGTCAGTTCCATTACACAAAGCCCCGCG | | Cat | | | Human | |
| 1066 | cg04927173 | CGCCTGCCCGCTACAGCTGTGGAAGGTCACTCATTGGGGAAATATGTTTC | | Cat | | | Human | |
| 1067 | cg04927884 | TTTGTCTGCAGTGAAAGGCTTTTGTTATTGCTTTTCCCCTAAGGGTTTCG | | Cat | | | Human | |
| 1068 | cg04958428 | CGGGAGCTCGTCCATGGTGATGCCCTGAGATTTGAGGTGCTTCTTGATGC | | Cat | | | Human | |
| 1069 | cg04991486 | CGGCAGAGCCCTTCTGGACAGCTCCCGCTCACCCAAACAGAAGACGTCGG | | Cat | | | Human | |
| 1070 | cg05311559 | CGCTAGGAGGCAGAACAAGATCAAGAAAGCTCAGCGAACTTGAACCTGTA | | Cat | | | Human | |
| 1071 | cg05362516 | GGTACAAGTAGCCCTGCGGATAGGACATGGCCACACACGGGCATGGGGCG | | Cat | | | Human | |
| 1072 | cg05576116 | CGCTTGACATCAAAGTCGAGGACACGAATGTAGTCTACCAGGACAGCAAA | | Cat | | | Human | |
| 1073 | cg06056217 | CGGAGCTTTTAAGGGTTTCTAATCCTGGAAGGCTGTCTGACATCACCCCG | | Cat | | | Human | |
| 1074 | cg06071123 | CGTAACCATGTAGGCCCTGCAAAGGCTACCAGTCCATCTCTCCGTCTTCG | | Cat | | | Human | |
| 1075 | cg06447962 | GTTGACAAGTTTTCTAACAGACTTTCTGAAATAATGCACATATATTCACG | | Cat | | | Human | |
| 1076 | cg06504202 | CGCCCCGATATTAATTAACGCATTAATAAGAGGGCGCCATTAGCCATGTG | | Cat | | | Human | |
| 1077 | cg06673387 | CGCCCACTGCAGACAGCTCCATCTTGCTCAGCGGCACCCACAGCCCATAA | | Cat | | | Human | |
| 1078 | cg07131451 | AGCCACTGAACCACAAGCAGCTTCGCGTTAACTGGAGTGCCTGGGAGTCG | | Cat | | | Human | |
| 1079 | cg07439538 | AATGACAATTAGCACTAATCTGGCACTTTATAAATTGTGATGTAGCCTCG | | Cat | | | Human | |
| 1080 | cg08253970 | AGGTACCTAGTTTCTGCCCCAGGTAGGTGAGGCTGTGATAGACCTTCTCG | | Cat | | | Human | |
| 1081 | cg17534919 | TTCGGGGCCGTAGCACTGTCTGAGAGGTTTACATTTCTCACAGTGAACCG | | Cat | | | Human | |
| 1082 | cg08357395 | CGCCCTGCTGCCAGTCCCACGTGCAGACGCACCTGCAGCAGCCCTCCACC | | Cat | | | Human | |
| 1083 | cg18434381 | CGCTCTCGCAGATCTACGACTGGATGGTCCGTTACGTGCCCTACTTCAAG | | Cat | | | Human | |
| 1084 | cg09535856 | CGCAGCTGATGCTGTTGACGGTTCTCTGACCCAGCAGTTGGAGGCCCTGG | | Cat | | | Human | |
| 1085 | cg19264807 | CGGTTAACAGTTTGAGGTCCAGGGTGCAGCTTTGGATCAGTTCATCTAAT | | Cat | | | Human | |
| 1086 | cg09966078 | CCAAGCAGTTTGCGGTGTGGCAGGCGGCCAGAAAAGCGTTTCTTTAAACG | | Cat | | | Human | |
| 1087 | cg10548614 | CGCTTCTTTGCTAAGCTGTTATTTTAACCATAAAAGGTAAACAGAAGGCG | | Cat | | | Human | |
| 1088 | cg10642116 | CGGACACAGACTTCCAGTCTTGCGAGGACAGCCTCATAGAGAATGAGATT | | Cat | | | Human | |
| 1089 | cg10717922 | CGGTCTCTGCTCATAGCTGAGAAGCCCGAAGGCGGCCATGATCTCTCATG | | Cat | | | Human | |
| 1090 | cg10751692 | ACTGACAAAAAATTTAAAAACCAATGTCCGGTTTCAACCAGTCAAAACCG | | Cat | | | Human | |
| 1091 | cg10752869 | CGGCCCGTCAGCAGCCCACATGTAACCGAACTGTCGTGTTTCATGGCTTT | | Cat | | | Human | |
| 1092 | cg10868173 | TCCAAGGCAAACATGCCAACTCGGAAGGCCAGGTTGTGGTGCTCTGGACG | | Cat | | | Human | |
| 1093 | cg10868866 | CGCTATGCAAATGACAGGACAAATCATGTGAAAAAGTCAGGATAAACTGC | | Cat | | | Human | |
| 1094 | cg10880576 | GCTTTTGCTATTCATAAATTTTAGGGCCTTAGATATTAGATACACTTACG | | Cat | | | Human | |
| 1095 | cg11370011 | TTCTTTTGGTGTTCAGAGTCAATATAATTTTCTAGCACCATCTGAAATCG | | Cat | | | Human | |
| 1096 | cg11527171 | CTCTGCTCTGTCTCCCCCTTTTGTAGGTGACTTTTTCACAGACTGCTACG | | Cat | | | Human | |
| 1097 | cg12001730 | CCTTCTTGTTTGCATCACCTCCAAGGAATTCATAATATCAAAGCTTAACG | | Cat | | | Human | |
| 1098 | cg12808529 | CGGCTCTCCAGGGGAAAAGCTGCAGTTCTGCTCTTGCAACACAAGGAGCA | | Cat | | | Human | |
| 1099 | cg21989707 | ACTTGGATGACATTTTGATTTCATCATTAGCATCCGGCGCCGGATTGACG | | Cat | | | Human | |
| 1100 | cg12892262 | TAACACATTTTCTGTGCTAAAAATTAGATTCTGATATATAATTATCAACG | | Cat | | | Human | |
| 1101 | cg22822366 | CGGGGGTGACAAGCCTTCCAGCCGCTTCCATGATTTTGTTGACCTTTGCT | | Cat | | | Human | |
| 1102 | cg14007606 | GCCACTGTAGGGGCACTTGTGCCTCTTTTCGGAGGCGTGTTTCCCCTTCG | | Cat | | | Human | |
| 1103 | cg14670966 | CGGCTGACTTGACAATGGGCCCGGGAAATCCCAAGTTAGTACTTAGTTTA | | Cat | | | Human | |
| 1104 | cg14689355.1 | ACAACAACAAAAGCGGGTTTAAATCACTGTCAAAATCACTGTCAGCTCCG | | Cat | | | Human | |
| 1105 | cg15791636 | CGCCCGGACCACCCCCCACGGGGTGCTCTCACTGCGCAGCGAGTCCCTCC | | Cat | | | Human | |
| 1106 | cg16111936 | CTGATTATTTTATTTGCAGCCATAATTATATTTCTTTCGGCTAAATCACG | | Cat | | | Human | |
| 1107 | cg16531937 | TCTTCTTGTCTTACGCTGCTTGCTCTTCTCTCCTTCTAAAGATGGTTACG | | Cat | | | Human | |
| 1108 | cg16947830 | CGTAAAAGACACATAATTTCAAGCATTTAATAAGGTAGATTTATTCTGGG | | Cat | | | Human | |
| 1109 | cg17179099 | CGGCCGTGGAACTAGAGTGGATCCCAGAGACTCTCTATAACACCGCCATC | | Cat | | | Human | |
| 1110 | cg17415555 | AGCAGAAATCCGCACTCAGGGTGTTGAGATACAAGGCGAGCCCCAGAGCG | | Cat | | | Human | |
| 1111 | cg25751973 | CGGAATAAATTCGGCGGGACTTTTAACAAACATGGAGGCAGAAAGTAAGT | | Cat | | | Human | |
| 1112 | cg17439607 | CGGCTAATTCTGTAGGAAAAGCACTAATTAGAAAAGCAATAATAATCGGT | | Cat | | | Human | |
| 1113 | cg17552603 | TTCTGCTAACTTTCAGACTTCATTGCTGACAACAGGCCTCTTGGCTTTCG | | Cat | | | Human | |
| 1114 | cg17659328 | CGACCGCGGCAGGACTCGAACCTGCAATCTTCTGATCCGAAGTCAGACGC | | Cat | | | Human | |
| 1115 | cg26073458 | CGCGCCGCCGCCCATGTCATTCCACTTCAAGTGACTTCATGTGATGTCAG | | Cat | | | Human | |
| 1116 | cg18369277 | CGCCGGTGCTAGCTGACCCACAGGAAACGAGCGCTGCTGACCTAGTTTGG | | Cat | | | Human | |
| 1117 | cg18809289 | GAAGTCGTTGTAGAAGGGCTTGTCCAGCAGGTGCTTCTCGCTGCAGCCCG | | Cat | | | Human | |
| 1118 | cg18897345 | CGCGGGGAGCATGCATCAAGGGTCCGTGGCAGTGAGAAGTCCCCACACAC | | Cat | | | Human | |
| 1119 | cg19631884 | CGCGGCACAGCGCCCCCTTGTCCATGGTCTCGAAGTCCGGGCTCTGCGCC | | Cat | | | Human | |
| 1120 | cg19759021 | CGGACGAAGGAATTGTTGGAAAATTTTCTCGGAGGTTCGTATATAAATGT | | Cat | | | Human | |
| 1121 | cg20096563 | CCCGCTTCCTGCTTTCTAATGTTCCATTGTGAGGAGCTTCCATTGTGACG | | Cat | | | Human | |
| 1122 | cg20123809 | CTATGTGACTTCAACCCCAAGAGCAGCAAGCAGTGCAAAGATGTGTCCCG | | Cat | | | Human | |
| 1123 | cg23160683 | CAACTCTCATTCATTTCGCCGGTTAACATGAGAGATCATGGCCGCCTTCG | | Cat | | | Human | |
| 1124 | cg20670041 | GAACAGGAGCAAGATGATTCTACTGTAGCCTGCCGTTTCTTTCACTTCCG | | Cat | | | Human | |
| 1125 | cg20718259 | GACAGGGTCACCCTTCACGCAGTCTTCCTTCTTACCATCTTTATGGTTCG | | Cat | | | Human | |
| 1126 | cg20751146 | GCTTGAAGTATGGCATGTTGCAAAGATGGTTTCTGCCAAGAAGGTACCCG | | Cat | | | Human | |
| 1127 | cg21334666 | GTGCAAACAGGCTTGCAGAACGCTGGCTGTGCAGCACGCCTCATACATCG | | Cat | | | Human | |
| 1128 | cg21458150 | AAGCAGACTCCTGGCTGTGACGCACTTCTGGCTGTCAGCCAGGAAGATCG | | Cat | | | Human | |
| 1129 | cg21475953 | CGGCGAGAGCAGAAACCACCTAAGAAGCCCAAATCTCCCAAAGCTCCAGG | | Cat | | | Human | |
| 1130 | cg21727711 | CGCCCGAAGGCCTCGATGATAACAGCATTGATTCCAATGTACATCTAATT | | Cat | | | Human | |
| 1131 | cg21998638 | CGCCCGAGGAGCTAACCATTTATGTCACACTGTGACAATTATTCTGCATA | | Cat | | | Human | |
| 1132 | cg22515103 | CGGCTGATGTATGGCATGTGGGGCTGGAAAGCTGCCATCATAGCCCAGTA | | Cat | | | Human | |
| 1133 | cg22793142 | AGCCACACCTCAGTGTTGCGGCAAAATAATTCTTGGCTCCCCTGGAAACG | | Cat | | | Human | |
| 1134 | cg24044043 | CGGCCAGAGGTGGTGGAGTGCAAAAAGCTTCCTACTTGCGACCGAAAAAA | | Cat | | | Human | |
| 1135 | cg23184246 | TGCTGGGGGGCTGGTTTAAGGGCTGGAACGAGTGCGAGCAGACTGTTGCG | | Cat | | | Human | |
| 1136 | cg23263602 | GAATGAAGGCTAATTACCTCAAGATCAGAACAAAATAACTCCTCATTTCG | | Cat | | | Human | |
| 1137 | cg25999777 | CTATCGCACACTGGAGCCAGTGCGTCCTCCAGTGGTACCAAATGATTACG | | Cat | | | Human | |
| 1138 | cg23582869 | CGAGGTTTTTTGTGACCTGTCCCTGCCCATCCCCAAGGTGGGATTCCAGA | | Cat | | | Human | |
| 1139 | cg26278617 | TATGACATTCCTAAAATATCTTTATTTAATAATGAAAACTTCATTATCCG | | Cat | | | Human | |
| 1140 | cg24028874 | CGTGCCAACAGTCGGCAAATTCACTGACAGTTCAAATGCGACATTTTTAG | | Cat | | | Human | |
| 1141 | cg24921129 | TCCTGGGATGTGACAGGTATTAATAACCCTATTAGCTGCAATCCCATCCG | | Cat | | | Human | |
| 1142 | cg24105939 | CGCCTAGAGACTTAAAGCTTTAATATGGATTAAAATAAGATAATCAATCA | | Cat | | | Human | |
| 1143 | cg25519108 | CGGCGCCGGATGCTAATGATGAAATCAAAATGTCATCCAAGTTAAATGCG | | Cat | | | Human | |
| 1144 | cg27286591 | AATGATAATACACAATTTTAGCACTGACGCTTGGCTGATTTAGGTGTTCG | | Cat | | | Human | |
| 1145 | cg25643003 | CCACACAGTACAGTAACTTATATACTTACCTGGAAGAACCCGCTTTGCCG | | Cat | | | Human | |
| 1146 | cg25250067 | TGTCTGAAGTTGAGAAAGCCAAGATCCGGGGATCGTACCTCACTGTCACG | | Cat | | | Human | |
| 1147 | cg25356878 | GCCTGCACTCACACACAGACAGAGGGGCATGATTAAAAGGCGAGAGCGCG | | Cat | | | Human | |
| 1148 | cg25492254 | TTGAGGTGCTTCTTGATGCAGGCCAGCCGCTGCACGTTGGGGCCCCAGCG | | Cat | | | Human | |
| 1149 | cg25560333 | CGGCCCTCGACCATCATGTCCGGGAGGAAATCCAGCTTGAAGGCAGAAGT | | Cat | | | Human | |
| 1150 | cg26284526 | CGTGGCGGCCGAGCTGTCTTGTCCAATTCACTGAAGCCAGGCCTCTAATC | | Cat | | | Human | |
| 1151 | cg26072973 | GAGGCAGCCATTTAACTATTCAGAGGAAACAGCTTCCTGGGGCACCTCCG | | Cat | | | Human | |
| 1152 | cg27313020 | GATCCCCACTGCTTAAGAGACGTCCAAAAATTCCTGGTGGAATGTTTCCG | | Cat | | | Human | |
| 1153 | cg26841850 | CGTGGAAGGTGAACGTACCCCACACAGGAGAAGTTTGATGTTGACAAACT | | Cat | | | Human | |
| 1154 | cg27391649 | CGGCTTTTCAAAGGCTCCCAAATTCTAAAAGACAATGACCCAACCCCCCG | | Cat | | | Human | |
| 1155 | cg05320568 | CAATTATGATTTCCATCTAAGCAACATATGCTGTTTGCTGGCCTCTTTCG | | Cat | | | Human | |
| 1156 | cg08578734 | AAGGAATATGCAGCACATTTTTGCCTTCTTCTGCACCGGTTTCCTAGGCG | | Cat | | | Human | |
| 1157 | cg06167345 | CGGCCACTTCGGCTGTGTCAATGCCATTGAATTCTCCAACAATGGAGGCC | | Cat | | | Human | |
| 1158 | cg17777878 | CGCCAAAAATAACCCGCAGGAAACGCAATTGTGCTCTGAGTCCAGGCAGC | | Cat | | | Human | |
| 1159 | cg17289116 | TGCATGTCCAAAGGCCTTCAATTCCACCTGCAAGTGATACTGAGAAACCG | | Cat | | | Human | |
| 1160 | cg09067976 | CGGCCCCTCAGGAGGCTGATTAGGCCACAAAGAGCCCCCATTATCCCGAG | | Cat | | | Human | |
| 1161 | cg13884249 | CGAGCGGAGCAAGGCGATTGAGAAAAACCTCAAAGAGGATGGCATCAGCG | | Cat | | | Human | |
| 1162 | cg10899907 | CGGGAAGAGAAGATCATCGAGCATTTCAAACGGTGAGTGACACGAGGCCC | | Cat | | | Human | |
| 1163 | cg06559544 | TACTGGATCTCCTCTGGGCAATGAAGCTGCCTCCTTTTTCTCCTCTCTCG | | Cat | | | Human | |
| 1164 | cg02982690 | CGTTAACCTGATTGGGTAGAAAGCGCTGTCCCAACAGGCGAGTCTTCTTC | | Cat | | | Human | |
| 1165 | cg17562187 | CGCGGGCTTGCCCTCCAGCCGGCCTTATGGCATTAAACCTGAACCGCTGT | | Cat | | | Human | |
| 1166 | cg03215611 | CGCAGGTCACTTCCTGTAGAAATATGGACTAACTTAAACCTCGTGAGTAC | | Cat | | | Human | |
| 1167 | cg20730297 | CTGTAGGTAAACACATTTTCTCCCTATTTAAGGCTGAAGCACTCAGCACG | | Cat | | | Human | |
| 1168 | cg04604143 | CGGGCTGCTGCACCGTCAGGATCTGAGACTGCTTCTCAGGGCTCACTCAG | | Cat | | | Human | |
| 1169 | cg19245820 | AGAGGGGCATGACCACTTCGGGCAAGCCTTTAAATGTGAGTCAGCTTTCG | | Cat | | | Human | |
| 1170 | cg18232211 | TTGATGGCCATGTGCTCCTGGATGTTGATGGTCATGCGATCGCCCCTGCG | | Cat | | | Human | |
| 1171 | cg00267231 | CCTTTGGGTTCAAGCTGCCTCTGAGAAGATGGCAATAAAACTCTAAATCG | | Cat | | | Human | |
| 1172 | cg11999944 | TTTCGCCCAAATCAATGTGGTTTCTTTGGAACATTTTCAGCAAAGGAACG | | Cat | | | Human | |
| 1173 | cg16032864 | CTCCAGGATAGCCAGGCTTTAATTTGTAATCTTAATTTTCATATAAATCG | | Cat | | | Human | |
| 1174 | cg09277592 | CGGCTCAGCTGTTCCACCCGCTTGCGCAGGCGGTCATTGTCACTGGTCAG | | Cat | | | Human | |
| 1175 | cg01608469 | GGAAGGCAGCACAGTCGGGGCCCCCATTGCCTGCAAAACTGGGGTCCACG | | Cat | | | Human | |
| 1176 | cg21530385 | CTAATCCCAACATGCCTGTTGTTTTGACATCTGGAACAGGGTCGCAAGCG | | Cat | | | Human | |
| 1177 | cg06956589 | AGGCTGCCCAGCTGCTGCAATCTGGCATGCATCATTCCTGTTCTGTTACG | | Cat | | | Human | |
| 1178 | cg01279020 | ATTATTACCCCTCTGTCAAGAATTAGACACAACAATCAAAGTAATCAACG | | Cat | | | Human | |
| 1179 | cg17318899 | TTTCTATTCAAATCCAGGTAGCGAGATATATGAATGGACTTTTCGAATCG | | Cat | | | Human | |
| 1180 | cg10425556 | CGGCACTTCCCCACCGCAGGCTTTGCTGAGCAGTTTTAGTATCAGGAGCT | | Cat | | | Human | |
| 1181 | cg13341299 | CGCATCTAGTCAGACGTAAATAATGACAGACGGAAACTATCATGAGGTCT | | Cat | | | Human | |
| 1182 | cg24918943 | CGCTTGAGTTTGACATCCAGGCACGGCCGCTAAATTGAGGCTCACAAAGC | | Cat | | | Human | |
| 1183 | cg21065165 | CGAGACAAATATGAGAAGAAGAAATACATGGACCGAAGTCTGGACATCAA | | Cat | | | Human | |
| 1184 | cg07903650 | GGTGGGGGCTGGGGCTGGCCAGCAGCTCCTGCTCATGGCCCGGGCCCCCG | | Cat | | | Human | |
| 1185 | cg23939166 | AGCCCATGATGTAATGGTGTATGTTAATCAGTAGCATGTGGGGAGCTCCG | | Cat | | | Human | |
| 1186 | cg22253255 | GGGGGATTTCGGGGGTTGCCCGTGACGTGGCTACTTGCGCAGCGGAAGCG | | Cat | | | Human | |
| 1187 | cg21315747 | CGCTATTTTGCTCATTTTAGAATATTGTGGGTAATGCTAGGCCTAAAGAA | | Cat | | | Human | |
| 1188 | cg07195315 | CGTGTCCTTTGTGATCCACTGTCAACTAGGCAAGGAGATCAAACACATTT | | Cat | | | Human | |
| 1189 | cg07480114 | CTACCTCCATGCTAGTATTAGGGTCAAGATCATCACACTCTGATTCCTCG | | Cat | | | Human | |
| 1190 | cg07514567 | CATTTCTGTGTGTCTGTGCCTCTTCAGTATGCAGAACGTGGTGCCCATCG | | Cat | | | Human | |
| 1191 | cg07157571 | TTAGAGGCTTTGGTTCTGCAAACTTGATGCTCGGCACTTGAACTGTCACG | | Cat | | | Human | |
| 1192 | cg03811583 | GCATGCCTAACATGATTCTTTCAGTCATTTATTATGCAGCTGCTACAACG | | Cat | | | Human | |
| 1193 | cg16089221 | TATTTGCCCTTTTCTGAACTCTACAATTTCCCTCTCTTGTAGCTGGTTCG | | Cat | | | Human | |
| 1194 | cg19629292 | TGTTTGCCATCAAAATGTGACAGAAGAGACACGCTGCATGGCTCGGAACG | | Cat | | | Human | |
| 1195 | cg22407807 | CGATCCCATAAATCCAAGAAGATAAAGTGCATTTTTATTAGTTGTGTTGG | | Cat | | | Human | |
| 1196 | cg16160648 | AGACAGGCGATTCCGATTGACTTCCTAATTACTGCTGCTGGGCTGCCTCG | | Cat | | | Human | |
| 1197 | cg25430089 | CGGTGCTGTAACAATACTCGATGATGTTTGGGATGACAGAAATTAATACG | | Cat | | | | Universal |
| 1198 | cg06392591 | CGGGCCCTGCAGTGTTGAGCCTAATGAATTCCGCTGTGGCTGCTCTGCTC | | Cat | | | | Universal |
| 1199 | cg05575054 | CGTCTTCTTCAACTGGCTGGGCTACGCCAACTCGGCCTTCAACCCCATCA | | Cat | | | | Universal |
| 1200 | cg19217984 | TGCTGCTGTAGGTGACCTTGAGCTCGTGCAGGTCTTTGTAATCCTCTACG | | Cat | | | | |
| 1201 | cg17679560 | TCTTTTTTATAAGCCTCTTCAAGCCCATTTTGCTTCGAAACAGGAAGTCG | | Cat | | | | |
| 1202 | cg24743148 | AAAACTATTACATTAATTAGTCAAATCCCACAAACCGTTAATGGTGCACG | | Cat | | | | |
| 1203 | cg21274598 | CTGGGGTAGGCCGACGTGGTGCCGTTGTAACTCAAAGTGCTGCTGGCGCG | | Cat | | | | |
| 1204 | cg18760617 | CGCGACATGCTGGCTCAAACATCAGCTGGGGCAGAATAAAAAATTACTAA | | Cat | | | | |
| 1205 | cg23559331 | GATGGTGTCCAGGAAGGTGTTTTGCGGGGCCAGCAACCCCTTCATGACCG | | Cat | | | | |
| 1206 | cg07705206 | CGGGTTTTGGTGATGACAAATGACTACTATTATACAGACATCAAGGGTAC | | Cat | | | | |
| 1207 | cg10141261 | CGAGAACAGCAAGCAAGTTCATCTGCTTACTGTCCACACGAAACAGAATT | | Cat | | | | |
| 1208 | cg12220591 | CGGACTGTAAAGTCACAGTTGACTGCTTTAATGTGGAACAGCTGGGAGGG | | Cat | | | | |
| 1209 | cg03411242 | GCCTTGGTAAAAAACACCTTCAAAGTCAAGCCATTGTCTCCAAAAAATCG | | Cat | | | | |
| 1210 | cg04947170 | CGGCAGGCCCGTCGCAGATGGCTGGCCACTAAGCTAATGGGGCAGCAGCC | | Cat | | | | |
| 1211 | cg12904782 | TTAAAGCAACCTTTATTTTATGGCAAATGTTGCAGTAATTGCCTTTTACG | | Cat | | | | |
| 1212 | cg14711273 | CGTGCAGTATAAAAGAATGTTTTTTCAAGTCAGTGAACTTAAGCTATTTT | | Cat | | | | |
| 1213 | cg15555400 | CGCCTGCAGTGGGTATTAAGCACTTAGGTTGGCAATGTAATGACACACTG | | Cat | | | | |
| 1214 | cg16313026 | TTCACTCTGGATTTTTGTTCTTATTTTTAACAGAGCCCCCTCGAGTGACG | | Cat | | | | |
| 1215 | cg16746452 | CGGACACACAGCACCAGACGGTGCAAGGGAAGTTAAGAAAAGACAAATTA | | Cat | | | | |
| 1216 | cg18299578 | AGGCGCACACTAGGGCTGTAAAAAAATTTTTGGTTTAACCTTTCCTACCG | | Cat | | | | |
| 1217 | cg23284252 | CGAAGCAAAATGGGCTTGAAGAGGCTTATAAAAAAGACTCAAAAGGTTTC | | Cat | | | | |
| 1218 | cg17927488 | CGCAGCGCAGGAAGCAAAAGCAGAAACAGACCATGCATCAGATGGCTGCC | | Cat | | | | |
| 1219 | cg18633258 | CGGCTTATCTCCACTAAAAGGGGAATTAGTAGTAAGAAATAATTTCAATC | | Cat | | | | |
| 1220 | cg22386201 | CGGCAGTGTCTGGAGGCTGTTTGGAGTTTTATGGGCTGTACAGTACACTG | | Cat | | | | |
| 1221 | cg26698347 | ATTACAGCGGATCACAGAAATGTTCCAGTCTGTGAGTCGGAATGCAGCCG | | Cat | | | | |
| 1222 | cg21510166 | TGGGCTGTGCCTGGCCTAAATTTTTCCTTTTACCATCTCTGCCTTATCCG | | | Mouse | Rat | Human | Universal |
| 1223 | cg24866418 | CGGCCGCCTTCTTCGTGCTGCTCTCCATGGTGCTGATCCTCGGCTGCATC | | | Mouse | Rat | Human | Universal |
| 1224 | cg05474883 | CGAGATGCTCACGCTCTCGAATGCTGCCGTTTGGTATGGTCTCTTCCAAC | | | Mouse | Rat | Human | Universal |
| 1225 | cg08402704 | CAAAAGGATATCACAGCTCATTTTGGTGGAAGGGTTTCCACGTCGCTTCG | | | Mouse | Rat | Human | Universal |
| 1226 | cg03957108 | TTAAGGAAACAGTGTGCCCTCCATCATATTCTGTTACCGTATTTTATTCG | | | Mouse | Rat | Human | Universal |
| 1227 | cg23687194 | CGGTGGCGCCAGTTAGTCTGCAGCGAGAAGGTTCACACACCCCCTCTATT | | | Mouse | Rat | Human | Universal |
| 1228 | cg12421693 | CAAACTGTCAATACCTTGAATGCTGCAGAATTTGAACAAATAGCCGTCCG | | | Mouse | Rat | Human | Universal |
| 1229 | cg09661809 | GTGCTGGCACTCATCGGCCTCATCTTCCTCATGGTGCTCTACCTAAACCG | | | Mouse | Rat | Human | Universal |
| 1230 | cg14237002 | CGGCCTTTGTTCCTGGAAACTAGGATTCCATGGCATATGGGGCACAGCAT | | | Mouse | Rat | Human | Universal |
| 1231 | cg15330117 | CTTAAGTTGCGTCGCGCCACAGCTGTCTGCGAACACTGAGCTGCCTGGCG | | | Mouse | Rat | Human | Universal |
| 1232 | cg01975510 | CGGGAAAAGGAGAACAATCCCAGCGGCAAAAAGGAGCTGTCTGAAGTTGA | | | Mouse | Rat | Human | Universal |
| 1233 | cg02040024 | CTTCCAGCCAGCCCCCGGCAACAAGACCGTGACCTACTTGGGAAAGTTCG | | | Mouse | Rat | Human | Universal |
| 1234 | cg20726505 | CATAGTTGGTGTGGTACAGCTTGGCAGCCTCCTGAGCCGGGAGCATCGCG | | | Mouse | Rat | Human | Universal |
| 1235 | cg27430422 | TACCTTGATCCTGTCATAGGACCTCTTCCATCCTTGTCATATCCCCCACG | | | Mouse | Rat | Human | Universal |
| 1236 | cg16376499 | CGTCCTAATTATTTCAGTTTGGGATGCCAGAGCTGTTTCCTGGATAGACC | | | Mouse | Rat | Human | Universal |
| 1237 | cg18577444 | ATCACAGGATAATTGCAGAACGCCTGCATAAGTAATTTCTTATGAAATCG | | | Mouse | Rat | Human | Universal |
| 1238 | cg01851187 | CGGCCTGCAGCCTGTGACAGAAATTAAAGGAAAGACCAGTGACCTCTTTT | | | Mouse | Rat | Human | |
| 1239 | cg13171587 | CGGCCATCGGGGGCCAGGGACAGGGACATCACATCCCCACTGTGTCCAGC | | | Mouse | Rat | Human | |
| 1240 | cg25966893 | ACTTCACAAATAGAGGAGCTCCTAAATGTGCCCAACAGCAAGGAAAATCG | | | Mouse | Rat | Human | |
| 1241 | cg13739934 | TGTGTCGCCTGCAGCTGCAGAGCAACCTCTTCGCCAGCCTGGACGAGACG | | | Mouse | Rat | Human | |
| 1242 | cg20665157.2 | TGTCAATAGAAATTGACAAACCTCATCTCCTAAATAGTGCAGCTGAGCCG | | | Mouse | Rat | Human | |
| 1243 | cg17624348 | GCTGTCTAATATGTTGAATAGTCTTTCATCTTGTCAAACACTTCCATGCG | | | Mouse | Rat | Human | |
| 1244 | cg17475635 | CTTCAGACATGTCAGCCTAGTTATTTGTTTTACATGCTGGGTGAGTTACG | | | Mouse | Rat | Human | |
| 1245 | cg16811337 | CGCACCACTTTCTTTGGTCCAGATTCCTGCATGGAAGAAATACCCTGTCG | | | Mouse | Rat | Human | |
| 1246 | cg02622074 | CGCGCTGGGCAGCGTTCCTGGAGCCTCCTAATAGCAGATATTCATGACTA | | | Mouse | Rat | Human | |
| 1247 | cg19843353 | CGCCCCAACCTGCAGCTGGCCAATATGGTCCAGGTGATTCGGCAGATGCA | | | Mouse | Rat | Human | |
| 1248 | cg07440346 | TGGGCTTTGAAATCCGCACCATGGGGGGGGCCGGGGGCAGTGGCCCCACG | | | Mouse | Rat | Human | |
| 1249 | cg08401965 | CGAGACAGAATGTGCTAACATCGGGATAAATTAGTGAAAGGGAAGAAAGA | | | Mouse | Rat | Human | |
| 1250 | cg13656158 | AGGCAGCTGATGGCCTTGAGCTCGAAGACAGGCCACTTGCTGCCCAGGCG | | | Mouse | Rat | Human | |
| 1251 | cg11939159 | CGCGAGGGCAAGCCCTTCAAAGCCAAATACAAACTGGTTAACCACATCCG | | | Mouse | Rat | Human | |
| 1252 | cg11115159 | CATGGTACCAGCATTTGGAATGTTCAGTAAGCCGCAGGCCCAGCCCCTCG | | | Mouse | Rat | Human | |
| 1253 | cg05193512 | AGGACTTCTGACTGTAATCCTAGCACGTCACTTTGTTGAAGGCAGACACG | | | Mouse | Rat | Human | |
| 1254 | cg22470076 | ATTACGCTCTCAGTTTGCTAGGTTGCCCCTTAGGTCCTAATCATGCAGCG | | | Mouse | Rat | Human | |
| 1255 | cg10103348 | CGACCCCGGAGACCCCAAGGCGTTTACTGAGATTAACAGGCTCTTGCCTT | | | Mouse | Rat | Human | |
| 1256 | cg01739251 | TGCCAAATTAAAATGAATCATTTGGCCCATAATGGCCGAGGCGCTTATCG | | | Mouse | Rat | Human | |
| 1257 | cg06000231 | CGGTGTCCAATAACTACATTGTGGGGAATAAGCCCCCAAAGGAATGTGGG | | | Mouse | Rat | Human | |
| 1258 | cg06479604 | TGGTACCACCTTCCTTTCTAAAATAAAATCTCTCTGGCATGAAGTCACCG | | | Mouse | Rat | Human | |
| 1259 | cg14472440 | GCCTTGGCAGCAGAAGGTGACATCTGGTGACTAATGGCCGCGGCTGCGCG | | | Mouse | Rat | Human | |
| 1260 | cg15651233 | GTAAGTAAACATACCAAAACCATATTTGCCTTGTTCAAGGTCCCAAACCG | | | Mouse | Rat | Human | |
| 1261 | cg10238356 | TAGCTCAAGACTGCAGTGCAGATGACGACAACTTCCTTGTGCCCATAGCG | | | Mouse | Rat | Human | |
| 1262 | cg11332125 | CGCCGTTCTCTGTTAGGAGGGTAATTTAGAGCAATTACACCGATTATTAT | | | Mouse | Rat | Human | |
| 1263 | cg12114804 | CGGAAGCGTTACAGGAAGACAGGTCTCAACTTAGGGATCAGATCACGTTA | | | Mouse | Rat | Human | |
| 1264 | cg07843568 | CGCCCCTCTCTGGGCTCTGGGGTCCCTGTCGGTGCCACGGACGCTTAAAT | | | Mouse | Rat | Human | |
| 1265 | cg27070699 | CGCTGCCTAGTGGAAATAATATAATACGTCATTAATATGGCGCCAAAAGA | | | Mouse | Rat | Human | |
| 1266 | cg19282646 | CGTGGAAATGATTTTGTGTTATCAGGCTCAGGGGATGACAGCATACAAGA | | | Mouse | Rat | Human | |
| 1267 | cg17201740 | CGCGGGGGCCAGCGCTGCGGCTGGATTCCCATCCAGTACCCCATCATTTC | | | Mouse | Rat | Human | |
| 1268 | cg13824077 | CGGGGTTAGCAATTTCACGGGTTATATATTTTAGAGAACCTCATTAAGTG | | | Mouse | Rat | Human | |
| 1269 | cg23507236 | GGTACCCAGCTAGCTGATGTGTCCCTATGCCGGTTACCTAGGAGACAACG | | | Mouse | Rat | Human | |
| 1270 | cg20842326 | CCGGTCTTGACTGGAAAAGCTGGTCATTAATTAGCCTCCTTTTGCCTTCG | | | Mouse | Rat | Human | |
| 1271 | cg06060272 | CGGCTCGGCATTTTGGAACAGAGAAGCCCCCGCCGTATATTTGCTAAAAA | | | Mouse | Rat | Human | |
| 1272 | cg13505775 | AGAACGTCGAGATGGAGCCCAACTCACTCCAGGTATTTCGCTCTCCTCCG | | | Mouse | Rat | Human | |
| 1273 | cg05809127 | ATGTTTAAAAACCTTAATTTGATGCTATGAGGCAACCCAATCTTTCCTCG | | | Mouse | Rat | Human | |
| 1274 | cg08334962 | GCGCTGGTATTGGGGCAGCGAGGGGGCAGTTCTCTATAACGCGGTCTCCG | | | Mouse | Rat | Human | |
| 1275 | cg18736324 | CGATCAGGATTACTATAGAGATCCTCGAGAGCGGACTTTACAACATGGGC | | | Mouse | Rat | Human | |
| 1276 | cg19279986 | GTGTTCGTGTCCTCTATGGCGCTGTCAATAAAGAACGGCAGTTTGAATCG | | | Mouse | Rat | Human | |
| 1277 | cg12277787 | CTGATGCAGACCCTCATGGAAGACTATGAGACACACAAATCTAAAAGGCG | | | Mouse | Rat | Human | |
| 1278 | cg15969104 | CGCATGATGCGACCCAGCAGAGACAGCAAAGTCATTGTATCCTCTATCAT | | | Mouse | Rat | Human | |
| 1279 | cg21047370 | CGCTTCAGCAGGGAGAGAAAAACCTGACACGCCATTTCAAACCGATTTCA | | | Mouse | Rat | Human | |
| 1280 | cg00045149 | CGCCTTGCAACTCAAGCCCGTGTGCGTGTCCGACAGCCTCAAGAAGGGCA | | | Mouse | Rat | Human | |
| 1281 | cg01469140 | CTCCCAGGCTGTGAAGGCCTCCGCTTACAAGTTCTCCTAGGGAATGGTCG | | | Mouse | Rat | Human | |
| 1282 | cg01996056 | CCATGTCCCATTCTCGAATGTTATTCTGTGACATATAACCAAGTCACCCG | | | Mouse | Rat | Human | |
| 1283 | cg02270531 | CGGGGCTCCGATCCTCCGCTTTGCAAATGAGAATGACCCCAATTTCCGAG | | | Mouse | Rat | Human | |
| 1284 | cg02439040 | ACATCCAATTTGGCTTGGCGTAATAAAATCAGCCGGAGCTCCCTTAAACG | | | Mouse | Rat | Human | |
| 1285 | cg02863784 | CGGCCATAGCCTTGGCTTCCCGGCGACCTCAGCGTGGTCACAGGGGCCCC | | | Mouse | Rat | Human | |
| 1286 | cg04294034 | CGGGATTGTGTAAAGATGATTACAGCCCTATCATTAATCAGATGCCTGCA | | | Mouse | Rat | Human | |
| 1287 | cg05570491 | CGTGATTTATATTTATTCAGAGGAATAACAGAGGGTGGATCTGTTCCCGG | | | Mouse | Rat | Human | |
| 1288 | cg05917369 | CGCACTTAATATTGATTTTGTTAGTGTTCAGATTCAATAATAAGGTACTG | | | Mouse | Rat | Human | |
| 1289 | cg05933419 | CGCTTTGAAGTTTTTGTCTAATGGCTCTAAACTGAAAGAAAATGTTTTCC | | | Mouse | Rat | Human | |
| 1290 | cg07747635 | TAAAATCCTTTTGATTGACGTTGTAGCCTCCGGTGCCCTGGGCTCAGGCG | | | Mouse | Rat | Human | |
| 1291 | cg08430237 | ATCGTCATGCTGAGGATGCTGCCACAGGCAGCCACTGAGGATGACGTACG | | | Mouse | Rat | Human | |
| 1292 | cg09403176 | CGTCCGGTGTCTGCGTGCCAAGCTGTATTACCAACTGCTTGGTCTGTGCC | | | Mouse | Rat | Human | |
| 1293 | cg11160926 | CGCTATCTGTCATGCATTTTAATTTCCTGAAACGAGAGACACAAGAGCCA | | | Mouse | Rat | Human | |
| 1294 | cg11950013 | TTCTGCTCTGTCGTCTCTCCACAGCACCTTTGTGAACTCAGGAATGTGCG | | | Mouse | Rat | Human | |
| 1295 | cg12559929 | TGTCTCCCACATGGACGATGGTGGAGCTGAAGCTACACTGACTGGTGACG | | | Mouse | Rat | Human | |
| 1296 | cg12757011 | CGCTGCTGCCTGACAGGACTGCCCAGGTCTCCTAAAGGTGAAGAGTTTCA | | | Mouse | Rat | Human | |
| 1297 | cg13561157 | CCATGGGCTTCCTGCACCAGCTGCAGCTGCTGCTCTGGAAGAACGTGACG | | | Mouse | Rat | Human | |
| 1298 | cg14179336 | CTCAAAATTAGCTTAATCACAGCTCATTTAAACTTATCTAAACAACAGCG | | | Mouse | Rat | Human | |
| 1299 | cg14754937 | CGACGTGGGCCTTTTGATCAGAAAAGAAATTAGCTCTAAAAAACAGACAA | | | Mouse | Rat | Human | |
| 1300 | cg15075184 | CGACACTAAAAGTGTGTTTATCTCTGTAGGAAGTAAACGGTTAGTCAATC | | | Mouse | Rat | Human | |
| 1301 | cg16198067 | CGCCTAGCATTGGAAATGAAAGTGACATTTACGCCACAACCCACGTGTGC | | | Mouse | Rat | Human | |
| 1302 | cg19364802 | CGCTAATGAGCTGTGCAGCCCATTGATCCGGAGAACACTTCCTAATTAAC | | | Mouse | Rat | Human | |
| 1303 | cg19904709 | CGGAGCAGGAAGTGACCCCAGCTTCCCGGGCCCCTCTAGCCTGGAGTCAG | | | Mouse | Rat | Human | |
| 1304 | cg20761322 | GTCTAGCTGCTCTTCGGTGAAGATGGTCTGCTTGTTCCCCATGGTGGCCG | | | Mouse | Rat | Human | |
| 1305 | cg21596828 | CGCAACACAGGGAAATTATTATTGTCAGAATAATAATGGTTTAGCATAAT | | | Mouse | Rat | Human | |
| 1306 | cg22324081 | GCCCTACCGGCTGGAGAAGACGCTGGGCAAGGGGCAGACAGGTGCGTGCG | | | Mouse | Rat | Human | |
| 1307 | cg22560467 | CGGGTTAGTGTTTCAGTGTTCTTAACTCCAATCTGCTTGTTCATTGTACA | | | Mouse | Rat | Human | |
| 1308 | cg23127199 | CGGAGCAACATCCCCTGTCACCTGGTTGATATCTCTTGCGGATTACCCTG | | | Mouse | Rat | Human | |
| 1309 | cg23493585 | TGACTAATTACACCTTGTCAATCACCGGCTATGAAGACATAAAACCAGCG | | | Mouse | Rat | Human | |
| 1310 | cg26191916 | TCCAACAGGCTGCATAATTTTTATGACCAATTATTCCATTTGTAATGACG | | | Mouse | Rat | Human | |
| 1311 | cg26245705 | ATTACTTAGTGAGCACATCATCATTTATTTGTAATTAGCAGCGAGGAGCG | | | Mouse | Rat | Human | |
| 1312 | cg07279255 | CGGCCCTGACTCACGTGCTTCCGGTTTGAAGGCAAAAAGTGTGCCTGGGT | | | Mouse | Rat | | Universal |
| 1313 | cg24924928 | TAGGCCTCTCTGGTATCTCTGTTCTTTGGCAGAGCCTAACAAAGTGTTCG | | | Mouse | Rat | | Universal |
| 1314 | cg22882174 | CGGGGAGTATGTGTGTAAGGTTTCCAATTATATTGGTGAAGCTAACCAGT | | | Mouse | Rat | | Universal |
| 1315 | cg02753151 | CGGCTCTCAGGAGCGACCCTACTTCATGTCGCCCGAGCTGAGAGACATCC | | | Mouse | Rat | | Universal |
| 1316 | cg00195533 | GCCCTCAGCCTTGTCCTTCTGGAAAGCTCAGAGACATCTGCTGTGTCACG | | | Mouse | Rat | | Universal |
| 1317 | cg26844246 | CGCACTGAAACTTTGCCAAGAGTGCGCGGGCCCGACAGGCTCTGTGTCAT | | | Mouse | Rat | | Universal |
| 1318 | cg17589788 | AATGTGAGAGCCCTGAGGTGGACGAGGTGTTTGCCCTGACCCCCCAGACG | | | Mouse | Rat | | |
| 1319 | cg14238859 | CGGCGCATTCGGCAAGAGCTGCCTTTCATGGCCACAGAGAACATCATCAT | | | Mouse | Rat | | |
| 1320 | cg01933636 | GGTTAGCCGACACCTCTGCTCTGTGGAATTTTAATTAACTTCTCTCATCG | | | Mouse | Rat | | |
| 1321 | cg06230736 | ATGCATTCTTTCTGAAAGTATCAAGACGGCGCCAGGCAGCTCAGTGTTCG | | | Mouse | Rat | | |
| 1322 | cg20149183 | ATCAGGAGGGCAGCCTGCAAATGTCAGCGCCCGGAGAGTCCCCCACTCCG | | | Mouse | Rat | | |
| 1323 | cg12231969 | TGTCACGCTCAGCTGTGCCCAGAGCGTGAGGCTGGCAGCGCCTTTAATCG | | | Mouse | Rat | | |
| 1324 | cg03295832 | GGGTGCTCATGCTCAAGTACTTCATGTGCCTGGTGGTGGGCATCACGTCG | | | Mouse | Rat | | |
| 1325 | cg00158333 | CGCCCCACTTTCAAATCAAAATGGCTCAAGCCCCAGGCCCTGGGGGGGAG | | | Mouse | Rat | | |
| 1326 | cg09536021 | TTACTTCCAGTGAATGCTATCCATCGAGCATATTTTGTAGCTTCTCTTCG | | | Mouse | Rat | | |
| 1327 | cg02818198 | CGCCCGCTGTTGGAGGAAGAAACGGAGACTCAACATCCTGGAGGACCCCA | | | Mouse | Rat | | |
| 1328 | cg02800363 | GAACAAGGAGGCCAGAGCAGCTAATGGGAAACATGTGATTCTGCCTTGCG | | | Mouse | | Human | Universal |
| 1329 | cg22057106 | GTGTTTTGGCAACTCTAAGTTACCTAAATTAGTTTTGATCAACACTGTCG | | | Mouse | | Human | Universal |
| 1330 | cg03679521 | CTGGCATAGAGGCTGGTACCCCGCCTGGTAGAGATGCCACACTCGCTCCG | | | Mouse | | Human | Universal |
| 1331 | cg19411464 | CGTGTCGGCTGCTTCACTATAAAGCACACAGAAAAATCAAGATACACAAG | | | Mouse | | Human | Universal |
| 1332 | cg22416332 | CACTTTTCATTTTTGGGTATTAACAATCAGTCCAACCTGACAGACATGCG | | | Mouse | | Human | Universal |
| 1333 | cg15426572 | GAAAGCACTTGCTAAAATCTGATCTGTTACTTCAGCAGCTTTCAGACTCG | | | Mouse | | Human | Universal |
| 1334 | cg25139325 | TTTAATAACAGCCACTCACAGCCCAACGCCGTGTTTATATTTTACATTCG | | | Mouse | | Human | Universal |
| 1335 | cg19291010 | TTGGAATCCAGCTGGGGGTTTCCAGATGGGGACATCACTGTAGGGGGACG | | | Mouse | | Human | Universal |
| 1336 | cg25731502 | CGGTATTGATTAGTTTGGCATAGACAGATGCAGCAGTTTAGAGCAAAATC | | | Mouse | | Human | Universal |
| 1337 | cg21246299 | CGTCTGACCCGTCTGGTTTCCAGGAGCAACCAGACATGATGTAACACCCA | | | Mouse | | Human | Universal |
| 1338 | cg12923877 | CGCATGAAAATTAACTGGTGAGGGGCCTCATTGCTATTTTTAAATTGTAG | | | Mouse | | Human | Universal |
| 1339 | cg25824158 | CGGGGGCGGAGCTCTGCCTGCCAACTGAGGGGATACAGTCTCTCAGTGCA | | | Mouse | | Human | Universal |
| 1340 | cg12307242 | CGGGCTGCTAATATGATTTACCAGTCTACAGTCATGCACAATAAGTGTGA | | | Mouse | | Human | Universal |
| 1341 | cg26594414 | CGGCTGTGCTGCCTGTTGACCATCCCGCCCAGGGTCCACCGGCGATCCAG | | | Mouse | | Human | Universal |
| 1342 | cg27069680 | CCGCAACATCTCTGTGGAGACAGCCAGTCTGGATGTCTATGCCAAGTACG | | | Mouse | | Human | Universal |
| 1343 | cg13761591 | GGCTGCACGGACAAGATTGTCTGAGGCTCTTTTCACAGCATTTCCTGCCG | | | Mouse | | Human | Universal |
| 1344 | cg20895555 | CGACGCTCTGCCTAAATTAAAAAGCAACCAATCGGAACGGCCGGAAGGGG | | | Mouse | | Human | Universal |
| 1345 | cg06779064 | TCATCCTTGAACACCACGCTGCCTTCAACTAGTGCCTGGTCATCCATTCG | | | Mouse | | Human | Universal |
| 1346 | cg25651791 | TCGTAAAAGGTGAACTTTTGAACTTGGAAAGCGCCCTGGAGCAATTAGCG | | | Mouse | | Human | Universal |
| 1347 | cg26476447 | CGCAGCAGCTCATGAATATGAAACGCCCATGATGCCACCTCAGTGGACTT | | | Mouse | | Human | Universal |
| 1348 | cg04998737 | GCTCTTCAATGCTGATTATTTTATTTGCAGCCATAATTATATTTCTTTCG | | | Mouse | | Human | Universal |
| 1349 | cg01594275 | CGGGAGCAGAGAGCAGGTCATCAACGGCGACAAGGACATTCAATGATGCA | | | Mouse | | Human | Universal |
| 1350 | cg14676280 | ACACTCACTCTGCTGTGACCTAATAAGGTGATGGTGCTATACTTCAGACG | | | Mouse | | Human | Universal |
| 1351 | cg09363187 | GCGGAGGAGTTCAAGCGCAAGCACAAGAAGGACATTGGGCCCAACAAGCG | | | Mouse | | Human | Universal |
| 1352 | cg07564343 | AGGGGATTTTCTCCAGTGTTTCTGATTAACCCTGTTACATAACCCGCTCG | | | Mouse | | Human | Universal |
| 1353 | cg17049911 | CGCCGCCGGGATGAGCCGCCCCCTGGACACCAGCCTGCGCCTCAAGACCT | | | Mouse | | Human | Universal |
| 1354 | cg17708456 | TTTGGAGCGATTTATCACTTTGATTTGGAGATCTTGTCAGATGGCAATCG | | | Mouse | | Human | Universal |
| 1355 | cg19707231 | CCGAGAGACCCAAGGGAATGATTCGGTCAGGATCCTTCCGAGACCCCACG | | | Mouse | | Human | Universal |
| 1356 | cg15168457 | TTATACTTTCCTTCATATCATTTGTTCTGTCAGGTGTTCCCTGGCCATCG | | | Mouse | | Human | Universal |
| 1357 | cg20212950 | CGAAGGCACGATATGCGTGTCCATCCTTACCAAAGGATTGTGACCGCAGA | | | Mouse | | Human | Universal |
| 1358 | cg21126148 | CCGCATTATCAATGACCGGGAGATGAGGGACGCCATAATCCTCATCTTCG | | | Mouse | | Human | Universal |
| 1359 | cg15340018 | CCTCGGATATTATTCCCATTTACCAGGGCAAAACTCAATTCCATTGCACG | | | Mouse | | Human | Universal |
| 1360 | cg09661817 | TCACCCTTTACCTGGAGCAAACCAAAGCCTGGCCACACCCCTATATTTCG | | | Mouse | | Human | Universal |
| 1361 | cg00489428 | GAGGTAGACACGGTCACATTCGGGGGGGCATCTGAGTCTGGCATCTTCCG | | | Mouse | | Human | Universal |
| 1362 | cg00593462 | CGCCAAGTCCCAGAAGGAAAACCCGCTTTTTAGCGGGTAGAGGATCAAAT | | | Mouse | | Human | Universal |
| 1363 | cg01019040 | CGGCGTGGACACCCCGCAAGCCGGCTATTTCGGGCTCTTCCACTACTGCA | | | Mouse | | Human | Universal |
| 1364 | cg03588035 | CGTCCAGGCTGTGGAGGGAGACAAAACCGTGAGCCTCCTGGTTAATGCCA | | | Mouse | | Human | Universal |
| 1365 | cg04087693 | TTAACCTACCCAACAGCCCGTGAAGAGTTAAGTAAATGCGGAGAGAGTCG | | | Mouse | | Human | Universal |
| 1366 | cg04353319 | CGGGGGAGCACGGGGAGAAAAATCAATGTCTCTCAAAATATTTGCAAAAT | | | Mouse | | Human | Universal |
| 1367 | cg04680480 | CGGCACATTCTGGAGAGCGAGAAGTGCCAACTCCAGAGCCAGGTGGAGCA | | | Mouse | | Human | Universal |
| 1368 | cg06655463 | TTTGACTTAAAGTGTGTGCTATTTATTATAGCAAACATCAACAGCCTTCG | | | Mouse | | Human | Universal |
| 1369 | cg07292994 | TCAGAACAAAATGCCGACCGGAAGTGCAGCTGCAGGAATGACTCCCTCCG | | | Mouse | | Human | Universal |
| 1370 | cg07782608 | GAGGCTGATGGAGAACAGGCACTGAACGCTAATCCCTTTTTCTATTTCCG | | | Mouse | | Human | Universal |
| 1371 | cg09362546 | CTTGCGCGACGGAGAGCAGAAGTTGCCCAGGTTGACCGAGTTAAACATCG | | | Mouse | | Human | Universal |
| 1372 | cg10278760 | GCTCGGTCCATTTCTGGGCGCTGCAAGATCAGCAGAAACCTGAGAAAGCG | | | Mouse | | Human | Universal |
| 1373 | cg10810311 | CGGTATTGTTCTCTATTGCTGTAGAAAGACTGGTTATTAGCCATAAATGA | | | Mouse | | Human | Universal |
| 1374 | cg13160087 | AGGGGTATGATGTTTGCAATGCATGCATTTCAGCAGGTCCCATTAGTACG | | | Mouse | | Human | Universal |
| 1375 | cg16867657 | CGGCGGCTCAACGTCCACGGAGCCCCAGGAATACCCACCCGCTGCCCAGA | | | Mouse | | Human | Universal |
| 1376 | cg17776554 | CGGGGCTGTGAGTTTCAAATTAACCTTCCGCTTTGTTGCTGTGTAATGTG | | | Mouse | | Human | Universal |
| 1377 | cg18168101 | CGCCCAGGGCCGCCTCCGCCCCGCTTTGGCCCATCTGCCATCCCCTGGCC | | | Mouse | | Human | Universal |
| 1378 | cg18963985 | TTCGGATCGCAGAGCTTTCAGACAGTCTCAACTGATAGACAGACACATCG | | | Mouse | | Human | Universal |
| 1379 | cg19663246 | AAGTCACCGGAGGCTCTATGATGTGAAATGTACAATCGAATTAGAACTCG | | | Mouse | | Human | Universal |
| 1380 | cg19683912 | CGGGAGCATAAAGCAGAAAAAAAATTAATGGAAAGGCATTTCACCTCCTC | | | Mouse | | Human | Universal |
| 1381 | cg19719499 | CTGGACTATGTTCCGGAAACAGTATACAGAGTTGCCAGACACTATAGTCG | | | Mouse | | Human | Universal |
| 1382 | cg19927064 | CGAGTCCTCAAACCCGGGCACGTAGGAGTCGTCATACATGGGGGAGTCCG | | | Mouse | | Human | Universal |
| 1383 | cg20424997 | CGCTGCCTATTTTCGCATGTGGATTTCAGTGGCGATGGTAAAAGCAGTGG | | | Mouse | | Human | Universal |
| 1384 | cg21617338 | ACATTTTGCTAATTTTAGTTTTACGGTGGGGTAATAGCACATGCCATACG | | | Mouse | | Human | Universal |
| 1385 | cg22454769 | CTTGGGAGCACAGTAGTTATCGGGAGCGTCGCCTCCGGCGTGGGCTCTCG | | | Mouse | | Human | Universal |
| 1386 | cg23088328 | ATTTGATGCCATCAAAGGGCTGTAGATTGAAACCAGATGTGCTCGCCTCG | | | Mouse | | Human | Universal |
| 1387 | cg23510032 | TTTGCTGGCTTGTTAATTTTATCCCCACTAACTGTGATTTCTGATAGCCG | | | Mouse | | Human | Universal |
| 1388 | cg23783364 | GGGCCTGTGACAGCTGAAATGATTGCACACAATTGCCAGTCAGTCATGCG | | | Mouse | | Human | Universal |
| 1389 | cg26260350 | CCTGATGAAGATTGATCACCTTTCTACTGCCCTCCCCGGGTATGTGAACG | | | Mouse | | Human | Universal |
| 1390 | cg27228973 | CGTGCCGACAGGCAGACCAGGAGCTGCTGCCACATGAATCACTTGAACTT | | | Mouse | | Human | Universal |
| 1391 | cg13202239 | ATTTTCTGTCTGTGAAAGCCTTTCTTCCCACCTCTGTTCAGATTAGCCCG | | | Mouse | | Human | |
| 1392 | cg06810447 | CGCTGCCGTACAGGTCACTGGCCACGGAAAGGCTATCCTCTGACCCCCAG | | | Mouse | | Human | |
| 1393 | cg13459195 | CCAGATCGGCATGAAGAGGCTCAAGGTGCAGCTGAAGCGGCCCAAAGACG | | | Mouse | | Human | |
| 1394 | cg16762797 | CGGGGGTCCCCAGTCCCCGGGCCACGGATCCACCTTCCCATGTAAGACCC | | | Mouse | | Human | |
| 1395 | cg21623348 | GCCATCAAAAGGATATCACAGCTCATTTTGGTGGAAGGGTTTCCACGTCG | | | Mouse | | Human | |
| 1396 | cg08716535 | TGCCTTTGTTTTGCTCCAGAGTTCGCTCCAAATGCTCATCTTCTTCTTCG | | | Mouse | | Human | |
| 1397 | cg06358839 | CGCTGGGAGCAATAAGTCCATTTAAAAGCCCTTTATTTAAAAGGAACAAA | | | Mouse | | Human | |
| 1398 | cg23647082 | TTAGCCCCCTTATCTGCTGAGCAATATAGTGGCAGGGCTGACCTCTTTCG | | | Mouse | | Human | |
| 1399 | cg10566829 | ATTTTCAGTTTTTAAAAGCCACCTTAGCCCGCCATATTGAAACTGGCACG | | | Mouse | | Human | |
| 1400 | cg19870489 | CGGGTTGTGTTTCTTTAACCCCTGGGGCCTGTTGACACAGATTAGTATCG | | | Mouse | | Human | |
| 1401 | cg20595453 | TCCTGACTCAGAGATTCCACTGATGATTTCCAGGAACTACCAAAGCCACG | | | Mouse | | Human | |
| 1402 | cg17543458 | GCATGACAAATTATGAAACACATACTGTGAAAATTATGGACTCTGTCACG | | | Mouse | | Human | |
| 1403 | cg09207276 | AAGATTTATGGCTTTGAAGTTTGCCGCTACCCAATCATCAAAGAATAGCG | | | Mouse | | Human | |
| 1404 | cg19458915 | ATGAATCAAGAAGACTGACCAGAAACTGACTCCCCTGATAACTAAGGACG | | | Mouse | | Human | |
| 1405 | cg00519323 | AGGGGCTTCGGGCAATTCATCATGGAAATTGGGTTTCTTAGCAGACATCG | | | Mouse | | Human | |
| 1406 | cg21371906 | CGGCCTGCCTGCTTTTCCAGCTGGAGCTGTTCATTCAGAACAATTAGCAT | | | Mouse | | Human | |
| 1407 | cg18913902 | GCCAGGAGTTGCCACTGACTATGGAAATTGCCACACCAGGGCTATAAACG | | | Mouse | | Human | |
| 1408 | cg24643424 | ACGCTTTTGGTCAACTGTACTCTGTAGAAAAAACAGGCACACTTGTCTCG | | | Mouse | | Human | |
| 1409 | cg06129152 | CGGATCAAAGAACAGCAGCAGTTTTGATTTGTGTTAATTTTGCGTGTACA | | | Mouse | | Human | |
| 1410 | cg06699148 | AGTAGGCAGCTCCTGTGCTACCGTATTATAGGATTCCAGCGCTGTCGTCG | | | Mouse | | Human | |
| 1411 | cg07571117 | CTCCATGAGGTCACACTCCATGCTACTGTGGTATTCAAATTTCCGAGTCG | | | Mouse | | Human | |
| 1412 | cg25483422 | CATTCCGAATATAGAGAACCTCAATCTCTTTGTCCGTGGTGTTAACACCG | | | Mouse | | Human | |
| 1413 | cg08665877 | CGCTGCCTTTGCTTTAAAACAGTATTACTTGCGGTGAGTAGTAGTGACTT | | | Mouse | | Human | |
| 1414 | cg09731876 | TTAACTAACAACTCATTAGCTTAGTTGATGTTTTTGTCAATATTAATTCG | | | Mouse | | Human | |
| 1415 | cg10067218 | AGCCAAATGATCACCTTCACCTTTGCTAATGGAGGCGTGGCCACCATGCG | | | Mouse | | Human | |
| 1416 | cg07751402 | ACTCATCTCCAGGAAGGACTTCGAGACCCTCTACAATGACTGCACCAACG | | | Mouse | | Human | |
| 1417 | cg12476049 | CGTTGCTGATGACAGAGCCATTCTCACTGGGCGACCTGCTGGAGGTAACC | | | Mouse | | Human | |
| 1418 | cg10724086 | CGGCTAAGAGCAGGTGAGTCACAGCACCGGTTTATATCTTTATGACATAA | | | Mouse | | Human | |
| 1419 | cg20610710 | CGGGGTTGATCTTCCCGTAAGATTCACAGCAACACAGCCTCTTGACTTCC | | | Mouse | | Human | |
| 1420 | cg20682817 | GGCAGTTGCAATCAGTTTCCGAAAGGTCAGCCAAGGTCCTCCGGCTCTCG | | | Mouse | | Human | |
| 1421 | cg25579908 | TCATAATTGTAACTGAGGGTGACAGGGTCACCTTGAGCCCCAGATGCTCG | | | Mouse | | Human | |
| 1422 | cg14769599 | GGATCCCTAATAGGAAATGCTTCAACAAACAACAATGCAGCATTTGATCG | | | Mouse | | Human | |
| 1423 | cg13107358 | GTAAAAGCACGTTCATTTCCAGGCACTCTCATTCATAGAGCCAGCGGGCG | | | Mouse | | Human | |
| 1424 | cg13132708 | CTGTCACTGCATTCCTGCTCCCCCTCCCTCTAGCTGGGTTTGTCAAGTCG | | | Mouse | | Human | |
| 1425 | cg19331623 | GTGTAATATTAATAGTCATGAATATCTGCTATTAGGAGGCTCCAGGAACG | | | Mouse | | Human | |
| 1426 | cg24677551 | GGAGCAGCCATTAAAATGTCGCCCGGAGACAGTAATAAAAGGCTCGGACG | | | Mouse | | Human | |
| 1427 | cg25424749 | CGGAAAATAAAGTTCAGAGCTGATCAAATTTGAAAACAGATGTCGAATCG | | | Mouse | | Human | |
| 1428 | cg22025998 | CGGTAAATAGCTGCCAAATTATGGATCATGCCGAAGTGTCACAACTGCAC | | | Mouse | | Human | |
| 1429 | cg21249921 | CGGCGGAGAGAACAGCCAGTTGTTTAAAATCCTTCTAGAGCAGTTTCTAA | | | Mouse | | Human | |
| 1430 | cg16595936 | CCAGCTCGTCCCTTTCAGGGGGGACTCATGCATCAAACTTCAATTTTCCG | | | Mouse | | Human | |
| 1431 | cg14927488 | CGCCCTATTTTCCAAAGTCTAAAATGGACATATGTTATAACTGTCTTGGG | | | Mouse | | Human | |
| 1432 | cg24124633 | CGCCGTGTGGTGTAAACAAACCCTGGATATTTTATATGAATTAAATGTGT | | | Mouse | | Human | |
| 1433 | cg17325863 | TTCATTTCTCGCTCTACCTCCTGTTGCATAAAATGATGCGCTGAAGAGCG | | | Mouse | | Human | |
| 1434 | cg23662675 | TTCTATGCACAGGATGGCTCTTGCTCACCCGAGCCTCACAAAGATAATCG | | | Mouse | | Human | |
| 1435 | cg26375896 | TATTTTGCTGTGCAAAATTGTTTTATATCTTGAGTTACTAACTAACCACG | | | Mouse | | Human | |
| 1436 | cg08683365 | CTTGGCCATATATGATGATGGCATCACTTCCCTCACTGCCACAAGAGTCG | | | Mouse | | Human | |
| 1437 | cg09401976 | GGGCCTCGATGAGAGAGACGATCTCCTTGGCGAAGGGCAGGTTCTCCTCG | | | Mouse | | Human | |
| 1438 | cg21118011 | CAGCTTGGTGGAGGCCTTATCCACTCCCACTTGTCCTGTTTGGAGGGACG | | | Mouse | | Human | |
| 1439 | cg01153660 | CCGGGAATGCGATGCATTTGTGTAGCGTGTGCAAATGGGCGCCGCGGTCG | | | Mouse | | Human | |
| 1440 | cg20128948 | CGTCGCCCTCAATTCCACCGCCTATGATCCAGTGAGGCATTTCTCGACCT | | | Mouse | | Human | |
| 1441 | cg04084157 | AGCATTTCATTCATTCATTCATTCATTCATTTCCCGGAGCTCCGCTAGCG | | | Mouse | | Human | |
| 1442 | cg13585349 | CGGGGACTATCTTGCCAGACAGCACTCTGGAGCCTGCCTACCGCCCACTC | | | Mouse | | Human | |
| 1443 | cg12946524 | ATAACTACCAGGAGGGTTTGATGGGGGATTCCGCCATGTCAATGATGTCG | | | Mouse | | Human | |
| 1444 | cg00672891 | ATCCCTCCTTAGTCCTCTATGAACTTCATCAAAATGCCTCCTTACATTCG | | | Mouse | | Human | |
| 1445 | cg09010998 | GGATCATGGCGGATGGCCCCAGGTGTAAGCGCAGAAAGCAGGCGAACCCG | | | Mouse | | Human | |
| 1446 | cg18872272 | CCATAAAGTCCTTCTGATTCCCGCATCTCTTCATTCATTCTTGCTAAACG | | | Mouse | | Human | |
| 1447 | cg02572855 | AGATAAATAGCAGAGTGTCCTCCTCAAAAATATGATTAATACCCTACTCG | | | Mouse | | Human | |
| 1448 | cg12837065 | CGGCATCTGACTGTACCTGTCTTGTTCTCTGCAGCATCTGTCTGCTACAC | | | Mouse | | Human | |
| 1449 | cg23773983 | CGGCGGCGGCAGCCAACTGCTCCATATTTGGCTTCTATTACACATTTACA | | | Mouse | | Human | |
| 1450 | cg02793066 | CGCACTGCAGGAGAAGGACACACAGACCAAACTGGAGGCAAGGCCACCAG | | | Mouse | | Human | |
| 1451 | cg04585931 | CGCTCACACCAAAAGCCACACACAGAAAACCAACAACGTGGGATGGGAGG | | | Mouse | | Human | |
| 1452 | cg23849723 | CGTTCATCAGTGTCAATGGCACTGAACCCTGTTTCAGGCCCATCAGTGTT | | | Mouse | | Human | |
| 1453 | cg04147092 | GGGAGGCGTCGACGTCATAATCCCCGGAACGTAAACATTGTTGCCGATCG | | | Mouse | | Human | |
| 1454 | cg26022924 | AGGACCTAATAGGTTTCGCTCTTTTTGGAGCTCATCAGCTGCAGGTAACG | | | Mouse | | Human | |
| 1455 | cg10797933 | CGCTGGAGATCTCTGTTCAGTCTGTGGTGACAACATCGGAGCTGTCAACG | | | Mouse | | Human | |
| 1456 | cg02216125 | TTTGGTCCATTGTCAAGAAAAGCCTTGCGAGGAACCCAATGGTGTTCTCG | | | Mouse | | Human | |
| 1457 | cg03045876 | GGAGGAGTTCAAGCGCAAGCACAAGAAGGACATTGGGCCCAACAAGCGCG | | | Mouse | | Human | |
| 1458 | cg17620221 | CGTACCCCACACAGGAGAAGTTTGATGTTGACAAACTTCTGGTGGCAGCG | | | Mouse | | Human | |
| 1459 | cg26828755 | CGGTTAACCTCGCAGTACTAAAACTGAATGAACAAGGCCTGTTGGACAAA | | | Mouse | | Human | |
| 1460 | cg24205097 | GGGTCAAGTTTCAGCGGCACTCCGCCCCCAGGGTAGGTGTGAAAGACACG | | | Mouse | | Human | |
| 1461 | cg13235828 | CAGAACTGTCAACCATGCAGAGTGAAAGGATATCCCAATAGAATTACCCG | | | Mouse | | Human | |
| 1462 | cg03810913 | AGTTGAAGTGTCAGATTACATTGGCTATTCTATTTCCAAAGGGCCCCGCG | | | Mouse | | Human | |
| 1463 | cg26617601 | GGGAACCCAAGATGGGCCAAGTGGTGTATGTGCCGGTCAAGACGGGCTCG | | | Mouse | | Human | |
| 1464 | cg00793774 | CGCGCAGGGGCAGTCATTCAGATGAAATTCCAGTCCCCGGCCAGAGACAC | | | Mouse | | Human | |
| 1465 | cg14898663 | CGGGGCCAGTCTTCAGCATTAATAAAAGATTCCAAGAACAGGGTGACCTT | | | Mouse | | Human | |
| 1466 | cg18766286 | CGCGCTCTCCGTTTGTGAACCCATTACCAAAGAGCTATAGCTGTACTGAT | | | Mouse | | Human | |
| 1467 | cg02169296 | CGGGGCAAGTGTCTTCATGAACCCAGAGGATGTCCGGGAAGCACTACAAG | | | Mouse | | Human | |
| 1468 | cg03957964 | CCATGGTGACTTTCTTCGGGGGCCTCTTCATCATCTTGCTCTGGCGGACG | | | Mouse | | Human | |
| 1469 | cg14204200 | TACATGGACAGCCTAGACATTGTCACCAGCAAGGTGTCAAGCCTCTTTCG | | | Mouse | | Human | |
| 1470 | cg13529178 | CGGGAAGCTAAATAGTTGTACCTTGGACTACCTTCCCACAAGTGCAGGGA | | | Mouse | | Human | |
| 1471 | cg13694139 | CGACCGTCATCTGTAAAAAATGTCTCAGTGAGAGATAATCATCGGAAACT | | | Mouse | | Human | |
| 1472 | cg24932816 | CCACTGGGCGGCCGGATTGTTCAGGCGGAATAATAAAACCTGCCAATCCG | | | Mouse | | Human | |
| 1473 | cg16604235 | TTCCCCCTGAACAGTAACCTATTTGAATATGATTAAAGGGTTTCATTACG | | | Mouse | | Human | |
| 1474 | cg00027948 | CGGTCTTTGGCAATCAGGTCCTGGGCCTCTAATACATGGATCCGAAGCAC | | | Mouse | | Human | |
| 1475 | cg00597234 | CCACCTTGATGCAGTCCTCGTTGGCGATGTTGAGCACGTTGGTCTGCACG | | | Mouse | | Human | |
| 1476 | cg01292864 | CGGGGGAGCTGGCTCCAGCCCCAGGATACACCGAGGAAATTAGTTTGTCT | | | Mouse | | Human | |
| 1477 | cg01418317 | CGGGCGATTAGCACCCATTGCATGAATTATGAAACAATAACTTTCGGAAG | | | Mouse | | Human | |
| 1478 | cg01431979 | AATATTCATGAACAAAGTCTGGGGTGATCAATCTATCTTTATTGTCTGCG | | | Mouse | | Human | |
| 1479 | cg01464821 | AAGCCACCAGCATTGCCAAGCCCAAGCCGCCAACCAGAATGTAGAAGACG | | | Mouse | | Human | |
| 1480 | cg01695075 | CGGTTTCCATAGCAACCAGCCAACGGGCCACTTTCTCAGAACAATTCCCC | | | Mouse | | Human | |
| 1481 | cg01742897 | TGATAATAATAGGTTTCCCTGAAAGATACATTGTAATCCATTCACATCCG | | | Mouse | | Human | |
| 1482 | cg01767626 | TTGTCGATTTTGTTTGCACAGAGGACGGCGACAGCTTCCTCAGGCTGTCG | | | Mouse | | Human | |
| 1483 | cg02026974 | ACTCTAGGTATTTCAAATCTGATGCTTAGACCACTAAGCCATCCCCTCCG | | | Mouse | | Human | |
| 1484 | cg02046143 | TTTCCACACGTGGCTCCTCGCTGCTTTGTTGCCTGGGCCAGGGAAGGGCG | | | Mouse | | Human | |
| 1485 | cg02298641 | CGGGAAATCATTAAACAGGAAGAAAGTGTGGATCCTGACTACTGGGAGAA | | | Mouse | | Human | |
| 1486 | cg03069282 | CCTGCCAATCGCTCCTGGCACGAGCTGGGTAACAAATGCCTGTTACCTCG | | | Mouse | | Human | |
| 1487 | cg03334831 | CCTCCCGCTGCTTCAGCTTGAACTTCATGTTTCCAAAGTGCATGATGGCG | | | Mouse | | Human | |
| 1488 | cg03549582 | CGCAATTAAAGATGAACTTTGGGTGAACTAATTGTCTGACCAAGGTAACG | | | Mouse | | Human | |
| 1489 | cg03552156 | AAATAAAGCAAATGAACCCACCGAAAACTGCTTGGCAAATATTTTTCTCG | | | Mouse | | Human | |
| 1490 | cg03746976 | CGCAATTGTATTCAAATCAGGAAACGGACCTAGGATGGTACAGGGATGTG | | | Mouse | | Human | |
| 1491 | cg03786140 | CGAAAACTTCAGGATGCAGCAGAACAACTAAAACAGTATGAAATAAACGC | | | Mouse | | Human | |
| 1492 | cg04288975 | CGAGAACACCATTTCCTACGAGACCACTGCCGCTGGGTGCATATTCAATT | | | Mouse | | Human | |
| 1493 | cg04787627 | CGCCAAGAGAAAGATCTTGGCCGTCTTCAGGTAGACATGGATGAACTTGA | | | Mouse | | Human | |
| 1494 | cg05473517 | CGGCGACGGGGCCCATCAGTAACTATTACGTGGACTCGCTCATCTCTCAC | | | Mouse | | Human | |
| 1495 | cg05675336 | TCTCCTGTACACATCATAGCCACTAGCAAAAGTTTACATTCCTATGCACG | | | Mouse | | Human | |
| 1496 | cg05791872 | GCAGCTGCCATCTGCACTATTGTTTCTATTGCTGATTTATTAAAGTACCG | | | Mouse | | Human | |
| 1497 | cg06144905 | CGGGGCCCTCTAGAATCTCTAAAGCAAATCAGGCTGAAGAGGGGAAAACC | | | Mouse | | Human | |
| 1498 | cg06488465 | CTATAGAAAAACCGCCACAGACGGCCGTGGCAGCTGTTGCTAAGGAGACG | | | Mouse | | Human | |
| 1499 | cg06767010 | TTCCGCGGCTATTGGATTAGTGGCCTTCAGGGATGAGCTCAGCCAGATCG | | | Mouse | | Human | |
| 1500 | cg07589899 | CCTGGAGCATCAGGGCGGTGCCTTGGAGGGCAGGATGATTTCGGGCTGCG | | | Mouse | | Human | |
| 1501 | cg07797757 | CGGGCAATTCATCATGGAAATTGGGTTTCTTAGCAGACATCGTGTGAATC | | | Mouse | | Human | |
| 1502 | cg07978595 | CATCTTGTTGGGGCTCTGCTGGATGGCCATGGTGATGAGCGAGATGTACG | | | Mouse | | Human | |
| 1503 | cg08179663 | ACGGCAATATGGCGAGAATGCCTGGCAGCGGGGACTGTAACACCAGCGCG | | | Mouse | | Human | |
| 1504 | cg08536905 | AACTATAGCTTTAAGGTATGCATACTCATAGCCATCTATATCCAGCTTCG | | | Mouse | | Human | |
| 1505 | cg08659673 | TTTGAACTGAAAAGCTCTCAGTCTAACTTCAACTCACTCAAATCCGAGCG | | | Mouse | | Human | |
| 1506 | cg08992759 | CGCGGCCCACACCTGGGACAATGAGCAGGAACCCAGGTGAGCAGTTTGTG | | | Mouse | | Human | |
| 1507 | cg09019552 | ATTTAGTGGGGGGCTCAATGGCGTACTCTCTGAAGGTCACTTCGCGCACG | | | Mouse | | Human | |
| 1508 | cg09075239 | CGAAGCAGTGGGGGCTGCCAAGGAACATGATTTACTCCTTGAGCATGCAC | | | Mouse | | Human | |
| 1509 | cg09268729 | TTATCCTTTTAATTACAAGCTGTAGACAGGGCCAACTTCGAAGGTCTTCG | | | Mouse | | Human | |
| 1510 | cg09691053 | CGCCCAGTGAGAATGGCTCTGTCATCAGCAACGAATCAGGGAAGCCCAGC | | | Mouse | | Human | |
| 1511 | cg09810328 | CGGGCAGCTGCTGTCAACAGTAGTGCGGACTCTTGCCACCCAGAGGTAGT | | | Mouse | | Human | |
| 1512 | cg09837345 | TACCTTGAGCTTCACTTCCTGATCCACCTTCAGCAACGAGGCCATGGCCG | | | Mouse | | Human | |
| 1513 | cg09878190 | AGTAGTGGAAGAGCCCGAAATAGCCGGCTTGCGGGGTGTCCACGCCGTCG | | | Mouse | | Human | |
| 1514 | cg10005379 | ATTGCCTTTCTAATTGATGGCTCCAGCAGTGTTGGAGATAGCAATTTCCG | | | Mouse | | Human | |
| 1515 | cg10083251 | TCTCCCACTTTTCCATAAGCAGAACAAGAACCAAATCAAACGTCTTAACG | | | Mouse | | Human | |
| 1516 | cg10084308 | CGACGCCTTCCGGGCGCACGCCGAGCAGAACTCCACCGACACCTTATCCT | | | Mouse | | Human | |
| 1517 | cg10152338 | GATGACCTAACACGCTCACACCAAAAGCCACACACAGAAAACCAACAACG | | | Mouse | | Human | |
| 1518 | cg10855531 | TCCACTGTGTCTGTCCTGTCTTGGGGCACAGCGGCAGAGGCATGCCCACG | | | Mouse | | Human | |
| 1519 | cg11453286 | CGTAACGCTGGCAATTTGTAGTACAAAAATGGATGCTTAATTCATGTCTT | | | Mouse | | Human | |
| 1520 | cg11804981 | CGGGCTAATGAGGTAAACTGAAGCGAGCCCTGTCCCCCCTGACAGGTTTA | | | Mouse | | Human | |
| 1521 | cg12091099 | CAGTGGTTAATTTGAAACTACTTCATGTCAGTTTCTTTCAGAGCAATACG | | | Mouse | | Human | |
| 1522 | cg13265357 | TCAGCACCATCTGCACAGGCTCATTTGTTGCTCCTGAGGCAGCCCACACG | | | Mouse | | Human | |
| 1523 | cg13283797 | GGGCCGTTCTGTTGAGTGCACTTGTCTGACGTGACTGTTGAGGTGATCCG | | | Mouse | | Human | |
| 1524 | cg13324564 | GGTGTTTTATAAGAGCCTTTTTCTTCATTAAATGGCATTCATTAAGCACG | | | Mouse | | Human | |
| 1525 | cg13672945 | CGGTCCTGGGCCTGAGCATCCATGGTGGGATTCCAGTCGCTGTCATAAAA | | | Mouse | | Human | |
| 1526 | cg13772936 | CGGGGCAGCCTTCCTGACCGACTACAACAACCAGGCCGACACCACCTGGT | | | Mouse | | Human | |
| 1527 | cg13850544 | CGCACCTGCGGTCCGGAAGTTCTCCCCTGACCTTAAGTTGCTTAAGGATG | | | Mouse | | Human | |
| 1528 | cg14064625 | CGCTCTCTTTGTTGCTTATGCCGATAAGCATGGGTTTCCTTGGTGCAGTG | | | Mouse | | Human | |
| 1529 | cg14833518 | CGGCCACGCTCCACACCACGATGACCAGGGTCATGAAGCAGGCCACCAGC | | | Mouse | | Human | |
| 1530 | cg14877474 | CGCTTAAATATACACAGGAGGCAGGAGATCCTATAATCCAACCTCACATT | | | Mouse | | Human | |
| 1531 | cg15354625 | ATGCTGAAGACCATCAACCTACAGAATGAGGGCTTCACCTGCACCATCCG | | | Mouse | | Human | |
| 1532 | cg15514262 | GGGGGGTATCTGTGTCCCAGAGCCTCATCCCCGACATCAAACCCCTGGCG | | | Mouse | | Human | |
| 1533 | cg16023422 | CGCGCGCTGCCGTCCCGGATCAGCCAGAGAGACCATTAAAATTTTATGCA | | | Mouse | | Human | |
| 1534 | cg16335529 | ACCTGAGCAGCCTTGGCAAATGTTGGCCCATGGTAGCGGCCACCAATGCG | | | Mouse | | Human | |
| 1535 | cg18182637 | CGTGGCTCTCAAAGTTCAGACTCTTCTAGTAGCTGTATGTATCTCAATCC | | | Mouse | | Human | |
| 1536 | cg18560921 | CGGTAAGTACCACTTTACTGTTCATAAGAATTGCAGAGCAATGGTGCTGA | | | Mouse | | Human | |
| 1537 | cg18650762 | TGAGGTGATGGCAACGGCCAACTTCGGCAAGATCCAGATCGGGATTTACG | | | Mouse | | Human | |
| 1538 | cg18933331 | GGTTCAGCCACCTTCAAATACTGCATCAATGGCTCGTGCCTCTGCCTGCG | | | Mouse | | Human | |
| 1539 | cg19755705 | TAGCTACTGTTGCTCTTGCTGATGCTGTAGCTCCCGCTGCTCCTGCCGCG | | | Mouse | | Human | |
| 1540 | cg19899882 | CGGCCCTGGGGCAAGGACAACTACTGGATGCTCAACCCCAACAGCGAGTA | | | Mouse | | Human | |
| 1541 | cg20150793 | CGCCTACCTAGAAACATTTTGAAATGCCAAGTTGTTTTAAACTTGTATGA | | | Mouse | | Human | |
| 1542 | cg20303044 | GCAGGCCAAAAAACGCTATTTCATCCTGCTCTCAGCTGGCTCTTGTCTCG | | | Mouse | | Human | |
| 1543 | cg20464004 | CGCACAATAAACCCTGTCATATAAAAGCAAACTGGATAAAATAACAGAAG | | | Mouse | | Human | |
| 1544 | cg20516274 | CTGCGGGGGTGGGGTGGTGGTAGACGTTGGCCGAGCTCTTGGCCAGCACG | | | Mouse | | Human | |
| 1545 | cg20558754 | CGAAAGTTATTGTTTCATAATTCATGCAATGGGTGCTAATCGCCCGCTCG | | | Mouse | | Human | |
| 1546 | cg20646588 | ACTCTTCTGGCAAGGCATTGCAGGACCCCTACTTTCTTAAAATTCATACG | | | Mouse | | Human | |
| 1547 | cg20697952 | CGGAACAGGACGCCCTCTGATGTGAGCATTTGCCAAAAATATCTCTGTCG | | | Mouse | | Human | |
| 1548 | cg20709200 | TTAAGATCAAAATCTGCGAGGCCGTGAGCTTGAAGCCCACAGCCTGGTCG | | | Mouse | | Human | |
| 1549 | cg20740396 | GCCATGGCCCAGATTCTGCCAATTCGTTTTCAGGAGCATCTCCAGGTGCG | | | Mouse | | Human | |
| 1550 | cg21611883 | GTTTATCTTGGCACTGGCAAGTACAGCAGTCTCTTCGATGTCTGGAAACG | | | Mouse | | Human | |
| 1551 | cg21646906 | CAGTTGGAAATGATTCAAAGCTGTGGTTTCACATGTTATCTGTAAAAACG | | | Mouse | | Human | |
| 1552 | cg21812916 | GGGTGCTCCAGGTGTTTGCCCCAGCCCCAGACAGCCAGTGGGAGGTGACG | | | Mouse | | Human | |
| 1553 | cg21836538 | CGCCGCTGTGGCTGAGAGCGAAGTTTCAGAGACTCTTATTTAAACTGGGT | | | Mouse | | Human | |
| 1554 | cg21972990 | TTTCTGACTTCTTAACTTGTCAGCAAATCCCAGAGTTAAAAATGCACACG | | | Mouse | | Human | |
| 1555 | cg22571854 | TCAGAGCACTGCGGTATATACTCATGTCATGAGCATCATACCACTCATCG | | | Mouse | | Human | |
| 1556 | cg22598041 | CGATGTCTGAACAAGCTATTGAAACTGTAGCAATCAAGTGGACTCTGGTT | | | Mouse | | Human | |
| 1557 | cg23014365 | TTATTCTTGTGTGGCCCTCGTAACATAAATGTAAATCTTTTATGCTAACG | | | Mouse | | Human | |
| 1558 | cg23124451 | CGTCCGATCCTAATCTCCAGCTCAGGCAGGCTCGGCCGCCACTAGCATCC | | | Mouse | | Human | |
| 1559 | cg23252695 | CATGCGATCCAAGGCGAGGGCGAGGAAGCTAGCCAAAAGTAAGTCTCCCG | | | Mouse | | Human | |
| 1560 | cg23500537 | GCAGCCACACATCCAAGGCTGACAGGGCGGGCACTCTGCCAAGTCCTGCG | | | Mouse | | Human | |
| 1561 | cg23563241 | GCCCCAATATCCCTGCCTATAACATGCAAACTTGTGGGAACTATGGATCG | | | Mouse | | Human | |
| 1562 | cg23805159 | GCTGAGAGCTTTTGTGTTTAATGACCTTCAGCTACCGGGAGGCAAAGCCG | | | Mouse | | Human | |
| 1563 | cg24076142 | CGCCGAGCTGCAAACCACACCCGAGTCCGTCCAGCTGCAGGACAGCTGGG | | | Mouse | | Human | |
| 1564 | cg24363217 | CGCTTTCAGCACCACCATCCACGTGCCCAACATTGCCACAGGAGAGCAGC | | | Mouse | | Human | |
| 1565 | cg24930802 | GCCAAGCCTGGAGCCCGTCCAGGCTTTTCATTAAGGACATAATATTTACG | | | Mouse | | Human | |
| 1566 | cg24998102 | TTTCTCCCTTTCTAATGACATTTACCGGATCAAAACATGCTGTTAATTCG | | | Mouse | | Human | |
| 1567 | cg25248596 | CGGATATTGTTATATTATTCCACCCACCTTAATGCTGAGCCTTTAGGGGT | | | Mouse | | Human | |
| 1568 | cg25410668 | CACCGCGTGGAGTTGCTTGTTCTTTTACATAGGAGGTCACATTCTCTTCG | | | Mouse | | Human | |
| 1569 | cg25909521 | CGGATGTCATTATTCTTATAAACAACCTGTCAAAAGCCAAACAAAAGTGC | | | Mouse | | Human | |
| 1570 | cg26424339 | CGGACGAGCAGTTCACATTCCATTACGAAATTCTACACGGCTTCAGAGCC | | | Mouse | | Human | |
| 1571 | cg26426930 | CGGTGATTGAGCTTGTGCTCAATAAATATTCTTGCAGTATGCTCAAACGA | | | Mouse | | Human | |
| 1572 | cg26600422 | CGCAGAACCCTAGCTGTAATCACTAAACTTGATCTCATGGATGCGGGTAC | | | Mouse | | Human | |
| 1573 | cg26636682 | CGGTTTTTGTAAACTCTCTGGATATCATCGAGCTGACGTCATGCAGAAAA | | | Mouse | | Human | |
| 1574 | cg26637635 | AAACATTGTTTGCATTCTAGAACATGGAGAAATCTTTTCTCTCCATTGCG | | | Mouse | | Human | |
| 1575 | cg26650303 | ACATCTTTAGGGCCGCTCGGGTTAATATATGTCGTCAGAGGCTCCTCACG | | | Mouse | | Human | |
| 1576 | cg26821197 | CGACTGCTATGAACTGAAGCCACAGCCTAAACCAGGTAATATACCCTGAC | | | Mouse | | Human | |
| 1577 | cg27359471 | CGCCTACTAAATGCACAGTGCCATGTGCATAGGCGTTTATTTAATTACAT | | | Mouse | | Human | |
| 1578 | cg27702990 | AATTGTGCAAATGAACCTTTAAATCCGTAAATAGTGCCCCCACAATGCCG | | | Mouse | | Human | |
| 1579 | cg26709300 | CGGTGACAATCATCCAAGTAGGCCTGAAACGTCTTGGGCTTTGAAATCCG | | | Mouse | | | Universal |
| 1580 | cg11614630 | CGGGGAGACTGTTTAACTTCAGCGTTTCTATAGGAGCCTGTTGTAGTTTA | | | Mouse | | | Universal |
| 1581 | cg20766695 | CGCCCCGATGGAAATACACTGTAAGCACGACCCGTTTGCATCCATGCATA | | | Mouse | | | Universal |
| 1582 | cg07005811 | CGGAGGTCCTATCTGTGATTGGACAGTCTTTCTTTGCAAAGTGCTGGTGT | | | Mouse | | | Universal |
| 1583 | cg08666298 | CGTCATGTCGAGACCTGCCAGAGAGAGACACATTCTCAAGTGAATCCTGG | | | Mouse | | | Universal |
| 1584 | cg05506474 | ATAAAAATCTTCCCCAACAGTGTGAAGGTTCCCCAGCGGAAACACGTTCG | | | Mouse | | | Universal |
| 1585 | cg19614548 | CGCCCCAGCTTCAGCTCGATGTCTCTGAGGTCTGGCAGCGGGCTGTCTTC | | | Mouse | | | Universal |
| 1586 | cg01571965 | CGCAGCGACACTTGTGAGTACTGTGGGAAAGTCTTCAAGAACTGTAGCAA | | | Mouse | | | Universal |
| 1587 | cg26917960 | CGTGTGACATTTTTATTCATGGTGGCGTACGCCCTTGTAATGATCAGCTC | | | Mouse | | | Universal |
| 1588 | cg01038105 | GTCCGTTGAGTTTCAAATCTGAAAATTTCTGTCTTTCAAGGTCAGCATCG | | | Mouse | | | Universal |
| 1589 | cg21602520 | GTGTGTGGAGAGCGTCAACCGGGAGATGTCGCCCCTGGTGGACAACATCG | | | Mouse | | | Universal |
| 1590 | cg24024857 | GCGCTAATTGCTCCAGGGCGCTTTCCAAGTTCAAAAGTTCACCTTTTACG | | | Mouse | | | Universal |
| 1591 | cg05933505 | CGTCTGACGGCACAGGTGCCTTGGCTCGATGTTCCTCGTTACTAACGGCA | | | Mouse | | | Universal |
| 1592 | cg21278764 | CGGCACTAAGCAAGCAGTTGCCAGTTGTACTAAAATTACTACCAACTTAA | | | Mouse | | | Universal |
| 1593 | cg18281552 | CAAAAATGAAACCAATTAAGGACAAACTTTGAAAGCCTCTAATTGCTGCG | | | Mouse | | | Universal |
| 1594 | cg27435767 | GCAACATGCTGAAATATGGGGTACATTTTAAATATCTTCATTTAAAAACG | | | Mouse | | | Universal |
| 1595 | cg06156376 | CACTTTCTGGTCAAAAGACTTGGAGACGAACGCCGTAAGTTCTTCCATCG | | | Mouse | | | Universal |
| 1596 | cg23298936 | AACAAACAAAAGCTGCTTCTTGACAGGGAGTCATTTATATTTTAGCTTCG | | | Mouse | | | Universal |
| 1597 | cg19323953 | CGCATCTGAACTGGAAAGGGAGAAGACAGACTAAATGAAATCCCATCCAT | | | Mouse | | | Universal |
| 1598 | cg08563010 | TTCTGTTTCACTTGTTGGCAAACTTACATTTCAAAACAACAACAAAAACG | | | Mouse | | | Universal |
| 1599 | cg22267613 | CGTGACGCTTCTGTCGTAATCCAATCAGGTTACATAGGTCCTAAACAAGA | | | Mouse | | | Universal |
| 1600 | cg26425570 | AGTGCCTAGTAATTGGTACAAAGGCACTTTCTGTTTCTATGCAACACACG | | | Mouse | | | Universal |
| 1601 | cg07476673 | TGTTTAACTTCAGCGTTTCTATAGGAGCCTGTTGTAGTTTAACTCCTGCG | | | Mouse | | | Universal |
| 1602 | cg23207462 | CGCCTGTGATTGACAGCTGGAGTGGCAGAAAGCCATGAGATTTGGTAGTT | | | Mouse | | | Universal |
| 1603 | cg18918349 | ACTTAGCTTTTCCCCCAACTCCTGGCGGGGTCTGACGTCAGCCATGTGCG | | | Mouse | | | Universal |
| 1604 | cg00258262 | CGGGATAGTAGGTGTCCCAAAATGTCTTAATTCAGAGAATTAGTGTTAAC | | | Mouse | | | Universal |
| 1605 | cg00587168 | CGCAGAATTAGCCTGAATTGCATGGTAACTGCTGCTGCTTTAAAATTTTT | | | Mouse | | | Universal |
| 1606 | cg11438204 | ATTTAATCCAGTCTTTGAGCTCATCCACAGTGACAAACCCGTCCTTGTCG | | | Mouse | | | Universal |
| 1607 | cg12713245 | TGCTATGGGGCCAGTTTCCTCCTCTTGTTTTTGGCAGGACGGCCAGGGCG | | | Mouse | | | Universal |
| 1608 | cg22204849 | CGTGCTTGGGGGGTAATTCTATTGGGATATCCTTTCACTCTGCATGGTTG | | | Mouse | | | Universal |
| 1609 | cg06147321 | CGGGTTTACTATTTAGTGTAAAGAGTTCCGATTTGGGTATATTTAAGAAA | | | Mouse | | | Universal |
| 1610 | cg08849105 | CGACCCCAGCCACAGCGACAACCTGCTGCACGGCCTCAACCTGCTGTGGA | | | Mouse | | | Universal |
| 1611 | cg05967003 | CGGAAATTGCCTTCATTGGATTAAGCAGCAGCCTTGGACGCGGGTTCTGA | | | Mouse | | | Universal |
| 1612 | cg05535461 | CGGAACAACAAATGATAGCTAACTACAGAGGCACATTTGCAGTAGTCACA | | | Mouse | | | Universal |
| 1613 | cg15343536 | ATCAGCTATTAGTGCCTAATCATATGGTCTATCGATCATGAGATTAATCG | | | Mouse | | | Universal |
| 1614 | cg10183248 | CGGAGAAGCCGACTGCCCCACAGACCTGGAAATGGCCGCCCCCAAAGGCC | | | Mouse | | | Universal |
| 1615 | cg08026739 | AAGGGTTTCTAATCCTGGAAGGCTGTCTGACATCACCCCGTTTCTTGTCG | | | Mouse | | | Universal |
| 1616 | cg02766818 | GTAAAACTGAAAGTATCCCACGCCAGAGTGAAGCCCGCTTTTTGACAGCG | | | Mouse | | | Universal |
| 1617 | cg18024395 | CGCTCCCCTTACAACACCTCAGTGTATGGTTGGTATAAGGGTTTGTATCA | | | Mouse | | | Universal |
| 1618 | cg12338698 | ATGGTGAGAATAAACCACCACAGGCCATTGTGAAACCCCAAATCCTGACG | | | Mouse | | | Universal |
| 1619 | cg02692845 | CGCAGACCTACATGAACGGCTCGCCCACCTACAGCATGTCCTACTCGCAG | | | Mouse | | | Universal |
| 1620 | cg24787338 | CGCGTAGCAGCCATCTGGCATCCTTATCCCGTCCAGAGCCATGGCTCCTT | | | Mouse | | | Universal |
| 1621 | cg05750171 | ACGTGAGACTTCTTCTTGAACATCTTGCCACAAATGCTGCATTTATGGCG | | | Mouse | | | Universal |
| 1622 | cg24866378 | CTCTCAGACAAGACTTGAACCTATGACCTTCACTGTGTACACAATAGGCG | | | Mouse | | | Universal |
| 1623 | cg12981876 | AATTGTTTGGCAAATTCTTTGGCTTCCAGCGTGTCCCCCAGGCAGGAACG | | | Mouse | | | Universal |
| 1624 | cg20265593 | CGGGGGCCAGCGCTGCGGCTGGATTCCCATCCAGTACCCCATCATTTCCG | | | Mouse | | | Universal |
| 1625 | cg27698932 | ATTTAGACTGACTGTTTGCACTGCAGACTGCGGCCACTGGGTTCCCGGCG | | | Mouse | | | Universal |
| 1626 | cg15702573 | GGTTCCAGAGGATTGGGCCCACAGGGGTCATGCTCAGTACAAGACAGACG | | | Mouse | | | Universal |
| 1627 | cg08981150 | TGACCCTCTGCTGCTGGGCCATCAAAGTTTGGTGCATCTTCTGCCAATCG | | | Mouse | | | Universal |
| 1628 | cg24975211 | CGAGACGAAAAGGAAAAGCAAACCAGAATCTTATTTATATTGTTATAAAA | | | Mouse | | | Universal |
| 1629 | cg04567783 | CGGACAGTGATTAATGATAGCAGAGCAGAGGGGTTAACACACTTCACTGA | | | Mouse | | | |
| 1630 | cg11565438 | CGGAGGATTTCCCAAGCAGTTGTTGCACAGGTAACAGGTAAGAGCTGAAG | | | Mouse | | | |
| 1631 | cg19399365 | TTCCCTCTCCAACGGGAGCGGAAAATGTGATTTGCTGTGCATTCCAGGCG | | | Mouse | | | |
| 1632 | cg16062897 | CAGAGACTGAACCCTTGATATAATAATCTACAAAGAATGGTGTGTGATCG | | | Mouse | | | |
| 1633 | cg04290831 | CTCCTGTCCATGTAAACATTCCTCTGACTTTCTTCCCTAAAATATAAACG | | | Mouse | | | |
| 1634 | cg05977669 | CGGTGAGAACACCGAGTGACGATCTGTTGCTTCCCCTGAGGTGGCTACAA | | | Mouse | | | |
| 1635 | cg24444575 | CGCGGATGGATGCTGCGGGAAGGGGCTGCCATTTGCTGCCCCTGCCAGCG | | | Mouse | | | |
| 1636 | cg13721678 | CGGGCACAGTACTCATCTAACACAAAGACTTGGCCAGGGCTAAACCATCC | | | Mouse | | | |
| 1637 | cg26834803 | AGCAGATCCACCACGGGGAGCCCACCGACTTTATTAACCTGCACAATGCG | | | Mouse | | | |
| 1638 | cg10859648 | TCATTTTGTGTGTAAATGAGCGTGGCATTCTGCTCACTTCTCTCTGCTCG | | | Mouse | | | |
| 1639 | cg00264615 | CGCAGCTGCCTTGAGGTTGTTTATTTCCCAGCGTTTTCTCAAGACTCCTG | | | Mouse | | | |
| 1640 | cg27050612 | GAGAGGAAAATCCCTGCATTTCCAACCACCTGATGCAACCAATGACAACG | | | Mouse | | | |
| 1641 | cg14817774 | CCTAGCTTCACCTTATTCTTTAAAGGGCAGAAAATTTGAGACGGTGATCG | | | Mouse | | | |
| 1642 | cg23394633 | CGCCCCTAACCACTGGGTCATCATTTATTCACAGGCCTGAAGTCCCTTAA | | | Mouse | | | |
| 1643 | cg04724146 | ACAAAAAGGAATTTACTGCAATTCCTTGCGGATTTTGCACATACAGCTCG | | | Mouse | | | |
| 1644 | cg16861303 | CCTTCCTACCTGAGGTGAACAGGACTATGGCCTTGAGGCAGCTGTACTCG | | | Mouse | | | |
| 1645 | cg15601638 | CGCTATCTCATGGACACAGGTACGGTGCCCAATTACACTGTAAACAGTTT | | | Mouse | | | |
| 1646 | cg23629098 | CGCTCCCTGCCAAATTGATTCAAATTGGCTTGGATATGAACATATGATTA | | | Mouse | | | |
| 1647 | cg12959926 | CGGTGGTCATGACCTACAGTATCACCTGCCCCATCATCGTGCCCTTCGGT | | | Mouse | | | |
| 1648 | cg09289224 | AGGACCTGGAAAGGAATGACGGCTCTCAGGAGCGACCCTACTTCATGTCG | | | Mouse | | | |
| 1649 | cg27496468 | TGCCTATAGAGCCACCAGTGACCCGTCAAAAAGAGTAGCATGGGAATTCG | | | Mouse | | | |
| 1650 | cg21082139 | ACAATGCTGCTGCGTCACCCACATGCAGCTCCCAGAAATGCTTTGCAACG | | | Mouse | | | |
| 1651 | cg16677609 | CGACCTTTGGGGCCTGGTGGTTGTTAATGCATGAGTGACTGCCTGCATTG | | | Mouse | | | |
| 1652 | cg16023341 | CGCCCGGCTCAGCTGCACTATTTAGGAGATGAGGTTTGTCAATTTCTATT | | | Mouse | | | |
| 1653 | cg17466220 | CGCTTTGTGGCCACGCTGGATATTAGTAGATGAGGATCATTCTGCTTCAA | | | Mouse | | | |
| 1654 | cg14800351 | GAAAAGTCTGTTGACTGGGCTTCTTGTAACACAATGTGGCCCGCTGCACG | | | Mouse | | | |
| 1655 | cg02806971 | CGCTTCGCGCACCCACCCCTGCCGAGGAGCCTACTTGCTGCAGCCCAATG | | | Mouse | | | |
| 1656 | cg24119607 | CGCCTCCCAGACCTCTGGGGCCATGAATCATTTATGAGGCAAAAATGAAA | | | Mouse | | | |
| 1657 | cg03380023 | GAAGTAGATAGCTCTATGCATCATTAATGAAATTTTTTAACACTTAAACG | | | Mouse | | | |
| 1658 | cg15052029 | CGTCAGACAAGTGCACTCAACAGAACGGCCCTTCAAATGTGAGGTAGGAA | | | Mouse | | | |
| 1659 | cg02758132 | CCTTAAGGAGTACCAGGCCAAGGTGACGCCCACGGGGCCCTGCAGCTACG | | | Mouse | | | |
| 1660 | cg21403130 | TTATAGTCAGTGGGTTGTTTGCGACCTTCTTAGCATACGCATCCAATTCG | | | Mouse | | | |
| 1661 | cg21872209 | CAATTACCATAAAAATATCTTTCTTTAAAATCCTGACCCAAAACACAACG | | | Mouse | | | |
| 1662 | cg02269624 | CGCATTGATTTAACACATCTTGTTTTCAATCATGCTTGGATTTTCTGAGC | | | Mouse | | | |
| 1663 | cg26300812 | GCTGCATTAAATTGTAAAATCGATTTATTGACCGGCAGGTGCGAGCAGCG | | | Mouse | | | |
| 1664 | cg11327853 | CGAGGAGGCTGAACACTGCATGAAGAGAGGACCGAGCCAATGAGAGAACA | | | Mouse | | | |
| 1665 | cg19327615 | TGCGTCAGCTCTGCCTTATAAATGGAGTGCATTCTATCAAAACCAGGACG | | | Mouse | | | |
| 1666 | cg16171324 | CGAAGATAATTGTTTCATCCACTTTGTCAAAGCTGAAATGAAAAGAGGGC | | | Mouse | | | |
| 1667 | cg08078389 | ATGCCACCTTTGGTTATTAACATGTCTCTCTCTACTTTTATGATTTTTCG | | | Mouse | | | |
| 1668 | cg27217092 | GCTCTCTCTGGGGACTTCCATGCAGGACATCCCCGGATTAGGTGACGACG | | | Mouse | | | |
| 1669 | cg03139948 | AGGACCCTTGATCTTCGGTGGGCAGTAGGAATCAAGGACCCTCAAATTCG | | | Mouse | | | |
| 1670 | cg10583293 | TAATTAGAAATGCTTTTCATATCTTCTCTGTGCAAGGGCTTAATCTGACG | | | Mouse | | | |
| 1671 | cg19356917 | CAGCGGCACACAGAGTCCCAGCCAATAACTGCACGAAGAATGAGCTAGCG | | | Mouse | | | |
| 1672 | cg07451598 | TAATTGCAATGAGGCCCTCTAGGCCGTGAATTAATGTGTCATAACTCACG | | | Mouse | | | |
| 1673 | cg19696052 | GGGGCTGTTGAGACTTTCTGGCATGGTGGTCATGGTCATCTCTTCTCGCG | | | Mouse | | | |
| 1674 | cg11717789 | TCTAGCCCAGAAGATGTCCAGGCTTTGGTGAGCGGGAAGCTTGCACTTCG | | | Mouse | | | |
| 1675 | cg13550191 | CGGCTGTCTCTAGTGTGTTATTGTTAGTGTTACATTACAAATCCCGTTTC | | | Mouse | | | |
| 1676 | cg21634324 | CGCCTGCCAGGCAGAGACAGATGGAGAGCTGACACTCCGGATTGCACTTG | | | Mouse | | | |
| 1677 | cg12880255 | GAAGAGCAGCAGCGGCAGCACGAAGGTGCAGAGCGCACTGGCCAGCAGCG | | | Mouse | | | |
| 1678 | cg11134207 | CGGGTTTTAGGCAAGCCAGGTGCATTCTGGATGTAGTCTGGTGTGGCAAT | | | Mouse | | | |
| 1679 | cg17946107 | CGGATTAGCAGTATGAAAGAAAGACACACAATAAGCTGGAATTGTTTTAC | | | Mouse | | | |
| 1680 | cg18834375 | CCAGACGCTTACTATGCAGTGTAAGCTCAGTTGGTTTACTAATCACCCCG | | | Mouse | | | |
| 1681 | cg11260565 | GGTTTCTGAACATAATAAAATCTTTGGCTTGTAGCGGCGGTTCAGTCTCG | | | Mouse | | | |
| 1682 | cg22123640 | ATCTACTTCGCCTCATTTGGTTACTAATTGGTTTCTTGTTTTATAAATCG | | | Mouse | | | |
| 1683 | cg14580567 | CACAGCCTCTCATGTTTCGTTCCCTCTTTTCTTCTTCTTTTTAACTAGCG | | | Mouse | | | |
| 1684 | cg25133549 | TTCATACCTGATGGAGCCTGCATCTGAGGATGATTCATTGTGCTCACACG | | | Mouse | | | |
| 1685 | cg17675822 | CGCCATGGAGAAATCTTGGTAATGACCCATTCCCCGTAAGCATCCCAGGA | | | Mouse | | | |
| 1686 | cg13651176 | CGCGTACATCACAGTAAGTTGTCTATTGATTTTCCTGAATGAAAATGGTC | | | Mouse | | | |
| 1687 | cg22561517 | CGCTGTAATAAGCAGTTATTATGTATGTGTAAAAATAATGAGAGGCCGTG | | | Mouse | | | |
| 1688 | cg11157816 | CGGGACCCCTCCAAGGACAGCAGCACCACCTTGTGGAGTACATGGAACGC | | | Mouse | | | |
| 1689 | cg00225449 | CGCAATCCAATCCATTCGGAGTTGAGCCTAGGATAGGAGGGGAAAGTCTA | | | Mouse | | | |
| 1690 | cg15051314 | TTGGTGTGCAGGGAGCCGCTGAACTGCATGTACTGCACCAGGTAGGAGCG | | | Mouse | | | |
| 1691 | cg22311716 | CGAAGGTGCTAAGGCCCAGAAGCTCAAAGGAGATACACATGTGGCCATGG | | | Mouse | | | |
| 1692 | cg22149945 | TTCTCTGCAGGACTGTGTGTTCAGCAAGGAAGCAGGACGCATGTGCTACG | | | Mouse | | | |
| 1693 | cg21740009 | CGTCAGGGTATCTGAGTTTCACAGAACAGACGGGGTGAGAGTTTTTGTGC | | | Mouse | | | |
| 1694 | cg01513560 | CGTCACTTGAACATGTGCCGTCCAGAGCTGGCCCTGTTCTAGCCGTCACA | | | Mouse | | | |
| 1695 | cg01701526 | CCTCCAGGAGCCAGAATCCAATTTCAGAAACCACAAAATCAACATAAACG | | | Mouse | | | |
| 1696 | cg27303748 | TGCATACCTGCAACACTTATGCTACCGCAATGACAAGGTGAAGACTGACG | | | Mouse | | | |
| 1697 | cg27186131 | TTTTTTCCCACAGGATTAGATCTGGGCAGGATTCATATTCATAACCCACG | | | Mouse | | | |
| 1698 | cg15069444 | CGGGCGCCAGCTTCCACAGCAGCAACTGTTGCTATGGAGACCAGGCTCCA | | | Mouse | | | |
| 1699 | cg06315860 | CGCCCCCGTCCACGGCCACGGAAGGAAGCCCGCGCCCCACCAGCCCCATC | | | Mouse | | | |
| 1700 | cg16331496 | AACACGTGGACTCTATAATCAACAAGCGGCTCTCAAAGTCCTCTGCCACG | | | Mouse | | | |
| 1701 | cg16976603 | CGCGTCAGGTGGTGGCGATGACTTAATTCCTAGCCCAAGAAGAATATAAT | | | Mouse | | | |
| 1702 | cg16018837 | CGCACACCTGCAGCAGGGACTTGACACTGGGCTGGAAGACCATGTTGGTG | | | Mouse | | | |
| 1703 | cg24831901 | CGGCTCCCGTTCTGCAGCAGCTCCTCAAACTGGATGGTGCAGTTGGCCAC | | | Mouse | | | |
| 1704 | cg13834844 | AAACTTCTATTAGTAATGGAGACCTCGCCGGAGACCTTCCGCGGAGAGCG | | | Mouse | | | |
| 1705 | cg08809534 | CGCAGAGCGAAAGCGTTTACAGACCTGAAAATTGTTGTTGAAGGCAGAGA | | | Mouse | | | |
| 1706 | cg03724931 | CGATCATTTCCTCACTTGTTTAAATAGTGCAGCTTGTGTTGGCAGTGCTC | | | Mouse | | | |
| 1707 | cg07898297 | CGGCGAGGGCCTCACCGAGGGCATGGACAAGCTCTATGAGATGATCCTGA | | | Mouse | | | |
| 1708 | cg14371358 | GTGCAGCAAACAAAAAGTGTGTGTCTGCGTGCCAGTCAGTCACTGCATCG | | | Mouse | | | |
| 1709 | cg17273372 | CGGGTTTGTCTTTAACCTCCAAACAGGGAACTTTAATTAAAGAGACAGTG | | | Mouse | | | |
| 1710 | cg09571697 | TAAACAAAACAGAAGGTTGTAACTGTCATAATTTGCATTGCACTTTCACG | | | Mouse | | | |
| 1711 | cg19703390 | CGGAAACCGCCCCCCGGCTGCTCCGATTCCCCTGGAGTGTGAAGCTGACC | | | Mouse | | | |
| 1712 | cg07230616 | AAGAGCACAGCAGTCCTACAAGTCCTTGGTGCAGATCCACGAAAAAAACG | | | Mouse | | | |
| 1713 | cg19834403 | CGGGATCTGTCTTTCACTTAGACTTAATTAAGAAACTTGCTGGCTTTGTC | | | Mouse | | | |
| 1714 | cg11218107 | GGGGCAGCCGTGGGCTTAGTCTTTGCCTTCCTCCTGGCCCTGACCACACG | | | Mouse | | | |
| 1715 | cg26352306 | TTCTGTATGTTTTTCCTTTAAAACCAAGAGCTGGGCAGGCATCCCATTCG | | | Mouse | | | |
| 1716 | cg21032275 | CGCCAGTCTGATGCCAGGACTGAATAAAGTGTATTTGCCCCGACCTTGCC | | | Mouse | | | |
| 1717 | cg12790413 | CTGATCTGTCAGACATTGATTATCATGAGCAGCTGTTGTTGGGTAGAACG | | | Mouse | | | |
| 1718 | cg04908805 | CGCCCTTAGATAAATAATTACACTCTTAAGCTGTGTCGAGTGCTGATACA | | | Mouse | | | |
| 1719 | cg13000461 | CGGGGGCCAGCTGTCCCAGTGGGGGTCATGTAGCCGATATTTATCTTGCA | | | Mouse | | | |
| 1720 | cg11464058 | AATCTGTAGATTCCCACAGCGCGTTTATATCCTCACTAGAGTGAGCTCCG | | | Mouse | | | |
| 1721 | cg26422625 | CGCTAAAGACCCCCTGACTTCCAGCAAGTCCACATTAAACATGTGACATG | | | Mouse | | | |
| 1722 | cg08230071 | AAGAGACTTGACTTTAAGAGACTCCTGTACTGACAACTCCATGCAGTTCG | | | Mouse | | | |
| 1723 | cg06545110 | TGTGCATTTAATGAAACCGTCTGGCTGTGATACCATCAGCTCCAAGCTCG | | | Mouse | | | |
| 1724 | cg07211487 | CGTTCAAGGCTAACACCTGAGCTTTGTGTAATGTAAATAAGACCTTGTGT | | | Mouse | | | |
| 1725 | cg06006726 | CATGAGGAATAGCATCTTCCAACTCATGATACACACCCTTGACCCACTCG | | | Mouse | | | |
| 1726 | cg08873407 | TTTAACTAGTGATTACAGACCGGCAACCTTCTAGCCCTGAATGTCAAACG | | | Mouse | | | |
| 1727 | cg24083274 | ATGGTCTGCTGCCCACCTAATTTTGCCTTATTGTCAGTCACTACTTACCG | | | Mouse | | | |
| 1728 | cg15936060 | CCATTATCTAGTTTCTCCTGCCTCCAGGCAGGGCCACATCATTGATTACG | | | Mouse | | | |
| 1729 | cg07966323 | CGGGTCACAGATTGAATCAATTGTCCAATCTTGTGAAAGTCATATCCTTG | | | Mouse | | | |
| 1730 | cg02785457 | TAATTATCCGTCTCATTTGAGAAGAGCAGCATTTGAGGCAGCAGCGTTCG | | | Mouse | | | |
| 1731 | cg08707617 | TTCAGGGCTTAATTACTCCTCCTTGATCCATGGGACTAGACACGACACCG | | | Mouse | | | |
| 1732 | cg17295628 | CGGGGAAATCCAATACGCTGAAAGGTTAATGCAATCAATTAGGGTGACAA | | | Mouse | | | |
| 1733 | cg08955338 | TATACATGTTCAAATAATTGCTCATCCTAATGTGCAGTGATCTGTATTCG | | | Mouse | | | |
| 1734 | cg04846076 | TCACTTTCACAACCCAGAACAAAAATCTCGCTAGATATGGCCTCAGACCG | | | Mouse | | | |
| 1735 | cg12247042 | CGGCCCTTCAGTGGCTTTGACCCAGATGACATGGATGTGGATGAAGATGA | | | Mouse | | | |
| 1736 | cg10270480 | TAGCTGAATGCCTCTCACAACCACGGGGGGAAGTCTGGAAGAAATCACCG | | | Mouse | | | |
| 1737 | cg01537787 | AGGCATCGATAGGCGGTGTAGGCAGCCTTCATTCAGTCCATCAGGGCCCG | | | Mouse | | | |
| 1738 | cg11226818 | CGCCTCCAGTTGAGGAGGCTGGGAGCCGCCATCTTCCCAACCCCCGCCCC | | | Mouse | | | |
| 1739 | cg02391262 | CGGGCCTGGAGCTGCCCGCAGCACAACGCCTACCGCCTCTACCACTGCGA | | | Mouse | | | |
| 1740 | cg05234920 | CGAGCCAATTAGACTTGTCAGACTCCAATCCAGTTGAGAGTGACCCCTGA | | | Mouse | | | |
| 1741 | cg10763467 | CGATGCATATGTTTAGAATAATGTTTCATTTAGCTGACTGCGAGTAATGC | | | Mouse | | | |
| 1742 | cg18961523 | TAAACAGGACTCCGTAAACATACTGACGAACTGGCCTATAGAGCAGAGCG | | | Mouse | | | |
| 1743 | cg22749775 | CGGAGACGTGATATGTCATATGATTAGCATCTTTCATATTTACAAGAGTG | | | Mouse | | | |
| 1744 | cg26925489 | CGGCTTCCCTAGAAGAACGGGCGGAGAGAGTTTTTTATGGCCCCATAAAA | | | Mouse | | | |
| 1745 | cg12121354 | GGGCTCCCTGTTCTTTCGCACCAGGCTGACAAATGTGTCAATCTCTGCCG | | | Mouse | | | |
| 1746 | cg02925207 | CGCATAAATCCAGATGACATAGATTTAGAAAATGAGCCCTGGTATAAATT | | | Mouse | | | |
| 1747 | cg12175065 | CGCACCGTTTATCTTGCTTGGATTCACCTTCAAGAGATTTCCGGACCCAT | | | Mouse | | | |
| 1748 | cg09201244 | CGCCAAGCTCTGTCCCCCAGGGCCGCCAGGTTGATGCTGATACCAAGGCT | | | Mouse | | | |
| 1749 | cg20463887 | TGATTTCTCTTTCTTCTTTGGAAATTTCATTGAGATGTCTCTGTACTTCG | | | Mouse | | | |
| 1750 | cg13369768 | AAACAAGGTTGGTATGGGCCCAACACAAACGCCCATATCGATGAGTCACG | | | Mouse | | | |
| 1751 | cg21016222 | CGTCAACCACCGCAAACACAACATTCCGCACTTCTGGCCCAAGCTGCTGA | | | Mouse | | | |
| 1752 | cg20916188 | ACATCTGGAACCAGTTGCAATACATCACTGTCAAAACTGCTTTCTATACG | | | Mouse | | | |
| 1753 | cg04342828 | CGCCGCCCAGGTGTTCCCCTGCAAGTACTGCCCGGCCACCTTCTACAGCT | | | Mouse | | | |
| 1754 | cg13125030 | CGGAGGGTCGCCAGGAAAGTGGACATTACCGCTTTAATTAACTTCGAGAT | | | Mouse | | | |
| 1755 | cg11341452 | CGGAGCAGGAAGACTGTTACTTACGTCGCCTGAGGGAAACCCACCGTCCA | | | Mouse | | | |
| 1756 | cg13636207 | GATTATTAAAAAGTTCATTTCCTGGCGAATCGGGTGACGTCAGGGGCTCG | | | Mouse | | | |
| 1757 | cg05166871 | CAGGCCTTTCAAACACCTTTTCCCTGCTCTATTCAAACTTGAATTCTGCG | | | Mouse | | | |
| 1758 | cg08001107 | CGCTTGGTTAATTCAGAGTAACAGATCTGCCCCCAAGTGAATTGGAGGCC | | | Mouse | | | |
| 1759 | cg16170739 | CGCTGTGTAGGGAGAAAAGATGTTGCAGTAGGTACTGGCGATCTACAGGA | | | Mouse | | | |
| 1760 | cg14431257 | AAAATGGATCAACCCTGGTGTGTAGCTAGCAAGCAATAACTGACTACTCG | | | Mouse | | | |
| 1761 | cg21752606 | CGGCCAACCTTACTGCACCCTTCAGCGTCCGGAGCTGGGCAACCATGTCC | | | Mouse | | | |
| 1762 | cg14398903 | TTTGTAAACTCCTAGGACGAGTGCCTTGCTTGAACCAAAGTGTTAAACCG | | | Mouse | | | |
| 1763 | cg15431157 | CGGTAACAGAATATGATGGAGGGCACACTGTTTCCTTAACTGCTTCTCAA | | | Mouse | | | |
| 1764 | cg14854610 | AGCTTGCCACAGTATGAAGTCCAGTTTGGGCTATATTCCCTTCCCTCTCG | | | Mouse | | | |
| 1765 | cg14732096 | AAACTTAAGAGCGACAGAACAAGGAACAGGCAGAGACTGCAAAGCCCTCG | | | Mouse | | | |
| 1766 | cg09522001 | CGTCAGAATCCTCACTGCTGCTGGGAAGGCGACCTCGTTTCATGATCTCC | | | Mouse | | | |
| 1767 | cg23443820 | CTGCCTTTGGTCACGTCGAAGACTTTCCCATTGACCGCGAGCAGGATGCG | | | Mouse | | | |
| 1768 | cg07219448 | CGTCCAGTAATTAATTTTCCTGCTGCCGGGGGGTGGTGGTTTTGACACAT | | | Mouse | | | |
| 1769 | cg15338129 | TTGCATGGGGACCCCCTGCTCACTCAGGACTTTCAGAGGAGACGCCTGCG | | | Mouse | | | |
| 1770 | cg07428697 | GTGCGGGGAGAACTGGTGCTTGAGGCTGCAGAGGAAGCTCCAGAGCTCCG | | | Mouse | | | |
| 1771 | cg20402664 | CTTTGGGGGGCAGCCCTGTCAGATTGGCCAGGTGGAAGATGTCGGCATCG | | | Mouse | | | |
| 1772 | cg19204561 | CGTGACATGAACTTTGTGTGTAAGAGTGGGTTCATTGAATTAAAAAAAAG | | | Mouse | | | |
| 1773 | cg02236977 | TATTACAGCTGACAAGAGGGTGATGATATTAGCCCATCATCATTCAGACG | | | Mouse | | | |
| 1774 | cg16555117 | TTGTACAAGATCCGCAGACGTGTAAATGTTCCTGCAAAAACACAGACTCG | | | Mouse | | | |
| 1775 | cg02339457 | CGGGCAGTAGTTTGTCACCAATTCCAAATGGCCAATCGTTAATACTAATG | | | Mouse | | | |
| 1776 | cg20665157.1 | TGTCAATAGAAATTGACAAACCTCATCTCCTAAATAGTGCAGCTGAGCCG | | | Mouse | | | |
| 1777 | cg08284222 | GACCATCATCTCAGGATGTCTCTTTCTGGCCGCCGATATCTTCGCCATCG | | | Mouse | | | |
| 1778 | cg01359371 | CGCCCTTCAGTGGCTGCATGTTCTCCAGCTGCTCCAGGAGGGGCCCGATG | | | Mouse | | | |
| 1779 | cg17185663 | CCGGACCATGTGCTCGGCGTGAGACAAAAGCAACAACAAAGGAAGTGGCG | | | Mouse | | | |
| 1780 | cg24959302 | TGAACAGTTGGATTCTTTTATTTCTTACAAGTGCTGGATTCGCCTTATCG | | | Mouse | | | |
| 1781 | cg19705440 | CGGCAGTTTTCGTCAGTTAATTAGGTTTAATTTACATAATTAGTACAGTA | | | Mouse | | | |
| 1782 | cg18278920 | CGGCTTACCAGGAAACGATGATGTGGCAACAATGCCATCTGGAGGCAAAG | | | Mouse | | | |
| 1783 | cg09802644 | CCTGAAGCGGAAACTGACAAATTCTGTTTAACAAAGACATTTGTCCCTCG | | | Mouse | | | |
| 1784 | cg03523182 | CGCCTTCCCAGCAGCAGTGAGGATTCTGACGACAATGGCAGTAAGTCCTG | | | Mouse | | | |
| 1785 | cg19046398 | CGGCCGCTGCCCAGGCGGAAGCCCTTGGAGCGCACCCAGAACTGGAACTT | | | Mouse | | | |
| 1786 | cg10456584 | CTCCAGCATGACCAGGCAAATCTGCTTGACACACTCGGTGACAGACTGCG | | | Mouse | | | |
| 1787 | cg17784271 | CAGATTTTCTTGAGTGAGCAAATCTGCCTGGCTTTTACACCTAGAAATCG | | | Mouse | | | |
| 1788 | cg00017571 | CATAACGGATGCATTTGCAGATTTGGAAAATTAAAGCCATCTATATGGCG | | | Mouse | | | |
| 1789 | cg07775532 | GAGCATCCTGTCTTCTGCTTGGCCTCCCAAGTGATGGATTTAACCATTCG | | | Mouse | | | |
| 1790 | cg11668811 | CGTCAAATGGCTCAGAAGGCAAAGTTTGGCTGCAAGGGCTATGGCCGTGG | | | Mouse | | | |
| 1791 | cg11311195 | CTCACCTGGGGGCAGCTGGAAGTTCTGGATGTTGGTCGGGTTCTGAGGCG | | | Mouse | | | |
| 1792 | cg11437639 | GCAATGTACTTCAGAAATAGGGTCCTGTTCATTCTTTGCTTTACAAATCG | | | Mouse | | | |
| 1793 | cg11477279 | GCATTATGTTTTGTCTGTAACAGCTGCTGTTTTATCTTTATTTCCTCTCG | | | Mouse | | | |
| 1794 | cg08509859 | CGGGCCTCCAAGATGATGTCTTGAAATGTACAGCCGTTGGAGGGCAAGGA | | | Mouse | | | |
| 1795 | cg20002283 | CGCGGCTCCTTTAAGAGCCCGGCTTTGCCTCCCGGTAGCTGAAGGTCATT | | | Mouse | | | |
| 1796 | cg22351850 | GTGTTCTCCAGCTGTTCCCGAGTAAAGAACCAGCGAGAAGAAGCTCCACG | | | Mouse | | | |
| 1797 | cg20426932 | TTGTCGAGCCTGAATATGCTGCTGTCAGACTCGCTTAATGAAAAGTAACG | | | Mouse | | | |
| 1798 | cg07221240 | GTTTCCTCAGGAGCTGGCGCAGAAAATTAAGGGCTACCAGGAGCAGATCG | | | Mouse | | | |
| 1799 | cg21159131 | CTCATGCATGCTGACCTGGAAATCCTCCTTTGCCTTAGCTGCGATAATCG | | | Mouse | | | |
| 1800 | cg15981030 | AACAGGGGGGAGGGGTAACAAACCAGCTCTCGTCCATCAAGGAGGGCGCG | | | Mouse | | | |
| 1801 | cg18131863 | CGATCCGTCAGTAATGGTGCATGTCGGGACCCTGGCGCCTCTGACAAGTC | | | Mouse | | | |
| 1802 | cg21667796 | TTGGAGGCTGCTCATGCCCCCCTTGAAGTCGTTTTAAAAATAAAACCTCG | | | Mouse | | | |
| 1803 | cg10437383 | CGCAAACATTGCGGTTTGGTTTGATTTAGTCTAACTCTTGTGTAATAACT | | | Mouse | | | |
| 1804 | cg17095500 | ATAAGAGTCTTTCCACTTCAATCTTCGAAGGTGGCTACAAAGCACATGCG | | | Mouse | | | |
| 1805 | cg13068696 | ACAGCTCGACCGTCATCTGTAAAAAATGTCTCAGTGAGAGATAATCATCG | | | Mouse | | | |
| 1806 | cg21153937 | GATGCTCCTTTCCCGGTAACTCGCCCTCTCAGGCAAACCTGAAGATAGCG | | | Mouse | | | |
| 1807 | cg13467605 | CGGAGCTCCAGCTGTTGTGGGAAGAGAGGACCAGCCGGCAACTTTTATCC | | | Mouse | | | |
| 1808 | cg21473019 | GTGCCTTGTCTTCTTATTCCATCCATGAATAGAAACGCTATCAGTCAACG | | | Mouse | | | |
| 1809 | cg11647864 | CGACCAGGTGTTTCCATTGTCTCTGGTAATACAGTGTTGGCCGCCAAGGT | | | Mouse | | | |
| 1810 | cg27589919 | TGGATCGCCAAATGGTCAGTGAGCTGTAAAATGTGCAACCCTCCTCCCCG | | | Mouse | | | |
| 1811 | cg13725272 | CGGCCCCTTTAAGCAGATACAAAGGCTCGGCTTGTTTTGTTACAGTTCAT | | | Mouse | | | |
| 1812 | cg13856487 | CGTAACATCTGCTTAATCGTACGGATCAAAATATGGAGACACAAAACATA | | | Mouse | | | |
| 1813 | cg19362114 | CGGGCCGCAGTTAAGGTGACGAGATGCAGCAATTCTTTATGAACATTTCA | | | Mouse | | | |
| 1814 | cg18772588 | TGATTTAATGTCTATACAATTAAGGCACGCGGTGAATGCCAAGAGAGGCG | | | Mouse | | | |
| 1815 | cg18123948 | CCTCAGACAGGTCTAAAGGTTAATAGAGCAATCACAGGGCCCTATTACCG | | | Mouse | | | |
| 1816 | cg18334881 | TGGGATTAATTACCACGTTCTGGTCCCTCAGACTGGAGTATTACTTATCG | | | Mouse | | | |
| 1817 | cg20119248 | CCAAAGAGCTCCACGGGACATCCATTGTGCCCAAGCTGCAGGCCATCACG | | | Mouse | | | |
| 1818 | cg18406787 | CGGAGCAGAAAATTACTAATAGAAGAGAGATAATGAATAGGCCGGCCAGG | | | Mouse | | | |
| 1819 | cg14705099 | CTGTGGCAGGGGTCTCGGAAACCATGGGTTATTGCAGTGGCAGGTGCACG | | | Mouse | | | |
| 1820 | cg17170462 | GCTCAGAGGCTCCAGCAGGGGCCAGAGCTTCAAAGTCAGAGAAAATCACG | | | Mouse | | | |
| 1821 | cg14924324 | AAGGAGACGTAGGGGGCTCCCCAGGGCCAGCGGGGCTGGTAACTGACACG | | | Mouse | | | |
| 1822 | cg00597719 | ACATGCATCAGCTGTTGGGCTTTTGTTACGGGTATTGTAGAACTAATCCG | | | Mouse | | | |
| 1823 | cg23564550 | AAGAGTGAGCCAAGGCAGTAAAATGTAGCTGAATGCCTCTCACAACCACG | | | Mouse | | | |
| 1824 | cg23627350 | AGGTGTGACTGCCATGCCTTGGGCTCCACCAATGGGCAGTGTGACATCCG | | | Mouse | | | |
| 1825 | cg01753578 | CGCGCTCCCTGCAAAGCAGCTCTCATTATCCCGGAAGTTGTGCTGACACA | | | Mouse | | | |
| 1826 | cg15710854 | CGTAGTAAATATTTGATACTGTTAAATATTTCACCAGTTTTATGGGGCCT | | | Mouse | | | |
| 1827 | cg25770163 | CGGCTCCGGCGCGAGTTGCCTCTCCGCTTCACGCTCGATTTCCAGGCATT | | | Mouse | | | |
| 1828 | cg24293580 | TAATGACCTTGAATCCCCAGCTGTTGGGAGGTGTCCTGAACTCGGCATCG | | | Mouse | | | |
| 1829 | cg02860346 | AGCAGCGGGAGCAGATTCGGGCAATTACAATCAAGTCAGGGCACACGCCG | | | Mouse | | | |
| 1830 | cg24184266 | GCTAATGGCCATGCATTATTCACCAGCCTAATTGCTCAGCCCCATGCGCG | | | Mouse | | | |
| 1831 | cg16960148 | CGTCAGGAAAGGCACTTCTTAGTACCAACCTGTAGAAAAAAGAGATAGGT | | | Mouse | | | |
| 1832 | cg27205180 | TCCCCAGTGCTTGTCATTTCCTGACATGCCTGACAGGATGGGGACAGCCG | | | Mouse | | | |
| 1833 | cg16625559 | CGATTGCTGGGGCCACAGTGTTGGTACTCTTATTTTGCTGTGCCACCTTT | | | Mouse | | | |
| 1834 | cg10587356 | CGCTCCTTCAGCTGGTGATCCACCTCAGGACCAAAGGCAAAATTATTCAC | | | Mouse | | | |
| 1835 | cg04099562 | CGTGACTGTTTGGAACCTACACCATAGTAAATTTGCTATTTTTAGTATAG | | | Mouse | | | |
| 1836 | cg16986847 | CGGACCACACCAATCCGTCTGCTGGTTGAGGCCAAAGCGAGCACGGCATT | | | Mouse | | | |
| 1837 | cg24226527 | TGAATCGGAGGTACTAAAAATACCCCCGCGACAGCCTGAGGAAGCTGTCG | | | Mouse | | | |
| 1838 | cg04802034 | GGTCACAATGCTTACACTGTATGCAGCAAACACCCTTACAAGTCTATCCG | | | Mouse | | | |
| 1839 | cg10198934 | TCACCGAGCTGGGGCCAACCCAGAAGTTCTCCTGCTGGATGTACTTCACG | | | Mouse | | | |
| 1840 | cg09585004 | CCACCACCTGCAAATACCCCAGGAAAAGCAGTTGGAAACAATAAAACACG | | | Mouse | | | |
| 1841 | cg12084468 | CGGCAAGACTAATTGCCATGCGCTTCCGCTTTGAAACTGGCAGCAGACAG | | | Mouse | | | |
| 1842 | cg03900224 | GTTCCATGCCCCAGCGGACTCCCATTAGGGTCTGCATCTCCTTGGTAACG | | | Mouse | | | |
| 1843 | cg04532288 | CTCATGTTTAGTAATTCAGACCTTTAATAACAGCCACTCACAGCCCAACG | | | Mouse | | | |
| 1844 | cg14603973 | CGGGCCCGAGCCCCTGTCCCACTTCGAAGTGGAGGTCTGCCAGCTGCCCC | | | Mouse | | | |
| 1845 | cg20231144 | CTTAGCCCATTGGGCCAAAGCACTGTCTTGGAGGTGACAAAGATGTCTCG | | | Mouse | | | |
| 1846 | cg15889617 | CGGAGACTCAACATCCTGGAGGACCCCAGACTCAGGCCCTGCGAGTACAA | | | Mouse | | | |
| 1847 | cg27702216 | CAAAGGGCAATATTCACAGTTTATCTCAGGGTGATGGATCTGAATGGACG | | | Mouse | | | |
| 1848 | cg19092999 | CTACCTTTAAGAATATTTCCTGTTTCTTCAACCCAACTTGCACACCTCCG | | | Mouse | | | |
| 1849 | cg20847714 | AGGGACAGGATATCACTCTTCTCCTTCTCTGGCTGGGCGAGCAGTTTGCG | | | Mouse | | | |
| 1850 | cg23402580 | CGGGAGAATAAGGCCATGCCCTGCCAGGTGTGCAAGAAAGTTCTAGAGCC | | | Mouse | | | |
| 1851 | cg24576158 | CGGATGAATAATATATGCATTTATGCAGAGGTAATTGCCTTAGCCAGGTT | | | Mouse | | | |
| 1852 | cg01675972 | CGGCAAAGTGGCCAGCAACAGAGTCCTGATCCAAATGGAGAAAGGGAACC | | | Mouse | | | |
| 1853 | cg21841556 | ACCTCGACCTTGAAGATGGTGAGTAGCCAGCCAAAGTACGATCTAATACG | | | Mouse | | | |
| 1854 | cg11832532 | CGCCGTACTTGTTGGAGCCCACATCGAAGAAGAAGCGCTTGTTGTCCACA | | | Mouse | | | |
| 1855 | cg19674753 | TCGGGAGGCTGATGCAACTTTCCCTTTAAGAAAGCCACCTGGGCGCACCG | | | Mouse | | | |
| 1856 | cg19683256 | GAAGGACCCTTTCCTCTCGCCATGCTAATTCAGAAAATCTGCTCTTAACG | | | Mouse | | | |
| 1857 | cg06663733 | AAAGAAAACCATGACAACTGTTTGTACCTCTTGCTTTATTATTACATCCG | | | Mouse | | | |
| 1858 | cg14159149 | GCCAGCAACTGTGCCAGGAGCGGCTGGCACGGGCTCGTGCCCAGGAGGCG | | | Mouse | | | |
| 1859 | cg17695612 | CGATTCAGCTCTTGCTGAAGGCCTTTAACTAGCTGTTTGAAATAAGAAAA | | | Mouse | | | |
| 1860 | cg15647529 | CGGCTTCTGCTGTGTTGCCTTGAAATTTATTATTCTGGAGATTTAAAGAA | | | Mouse | | | |
| 1861 | cg06885782 | GCGAGCTCGATTCTCAGCTGTCAGCAAAGGCAGCATTGATCTGCTGAGCG | | | Mouse | | | |
| 1862 | cg13275896 | TAATTACCTTTGCAGAGCCAGCAGATGGTGCAGAAAGCACTGCATTGTCG | | | Mouse | | | |
| 1863 | cg20735499 | CGGAACATAAGGTTTAAGAAACACACACACGTTTTCTGGGGTCTCTTGTT | | | Mouse | | | |
| 1864 | cg04317929 | CGGCTCTGACGAACAGACCTGTCCTGAGCCTGCCGGTCAGTGCATAGAAG | | | Mouse | | | |
| 1865 | cg19961913 | CGTGCCACCCAGCTGCTGCATGGTCACGTCCATCTGGAGCTCTCTAGCTA | | | Mouse | | | |
| 1866 | cg04801716 | GGGCTGTGGGTGCCGCTGAGCAAGATGGAGCTGTCTGCAGTGGGCGAGCG | | | Mouse | | | |
| 1867 | cg04121944 | CCTTGCGCTTGAAGCGCTGCTGGCACACCGGGCACTGGTAGGGCTTCTCG | | | Mouse | | | |
| 1868 | cg04407088 | TCATACCCAATTTATTACCGCTGAACATATGGCCAATATTTTGACTCACG | | | Mouse | | | |
| 1869 | cg04447890 | ACAGCTTCTTGGTGCTCATGTTTTCTATAATTGCCCCAGCCATTTTTGCG | | | Mouse | | | |
| 1870 | cg16181889 | CGCAGAAAATTCGCAAATCCTTTGATTGTCTGTTTAAAATGAGGAGGAAA | | | Mouse | | | |
| 1871 | cg01574836 | CACAAATGTCTCTTGCATTTTCTTGCCTCAGAAGTGAAACCATCATATCG | | | Mouse | | | |
| 1872 | cg26070145 | GCTTATTGTGTGTCTTTCTTTCATACTGCTAATCCGGTTTACACATCACG | | | Mouse | | | |
| 1873 | cg25452665 | AGGACCCGGCCGGCCGCCTGTTTATATTAGCGCGGTGTAAAATATTCTCG | | | Mouse | | | |
| 1874 | cg12502231 | AACCGAGATTGGAGTCATATGGGCCATAAATCATTGAGACATACTCTCCG | | | Mouse | | | |
| 1875 | cg12277614 | TACTCACTGTTCCTGACTGTGGCATAGCAAACACATCAATCACTCTGACG | | | Mouse | | | |
| 1876 | cg27269484 | CAGATCCTGGAGGCTGTGCTACACTGCCATCAGATGGGCGTGGTCCATCG | | | Mouse | | | |
| 1877 | cg10277837 | CGAGCTCATGGTCATTAATTTGTGAAGTGCAAAAATACTAATTTTTCTCG | | | Mouse | | | |
| 1878 | cg04561327 | CGCACACACAGGCTGCTGGGTAAGTGGCAAGATGGCTCCCCTGCATGTCT | | | Mouse | | | |
| 1879 | cg14593595 | TTAGTGGCCACAGCACGATTCTGAAAATATACCTTCCTTACGATTTAACG | | | Mouse | | | |
| 1880 | cg14689223 | GGGAAGGCAGCCAGGTTTCGGGTGGGCTGGTCCTGCAGGGACTGGGATCG | | | Mouse | | | |
| 1881 | cg00036788 | ACCACATGTTGCAAGGTTCAAATAGTGCCTAATGAAACAGTGACTAAACG | | | Mouse | | | |
| 1882 | cg00083068 | CGGAGGCATATGGTCTCGTACACAAGAAATAATGTTCATGTTAAATCAGA | | | Mouse | | | |
| 1883 | cg06637963 | CATTTAGACACAAGCGAGAGGATCATGGCGGATGGCCCCAGGTGTAAGCG | | | Mouse | | | |
| 1884 | cg14458704 | CGGCCCCAGTGTGAGCTCATACATCAGGGCTGACAGGCTCCAAAAGCCCC | | | Mouse | | | |
| 1885 | cg11882147 | GCCCGCGTGGTGGGGGCTGGGGCTGGCCAGCAGCTCCTGCTCATGGCCCG | | | Mouse | | | |
| 1886 | cg13529847 | CGAGGGGAAAAATTAATTTCTGCCTATCGTTAATTGAGAGAATTGTGTGG | | | Mouse | | | |
| 1887 | cg26575155 | CGGCATGACAGGCAGCAATCCTGTGTCCCCTGCCTCATCCAGTTCCCCAG | | | Mouse | | | |
| 1888 | cg07479479 | CGCGGTTTGTCCCGTTGACAGCGTAATTTAAAGAAATATATGGAAGGCTG | | | Mouse | | | |
| 1889 | cg17797814 | CGGCCGCTCCATAGGTCGAGAAATGCCTCACTGGATCATAGGCGGTGGAA | | | Mouse | | | |
| 1890 | cg01901658 | CGTTTGAAGAAACCATTATGACAAGCAGGTCTAGGGAAGCTGCTGCAAAA | | | Mouse | | | |
| 1891 | cg09522374 | CCCGCAATGAAGTCAGCACTCTGCTTATTAAGTGGAAAAAGCTGTGCTCG | | | Mouse | | | |
| 1892 | cg21141161 | CGCATGGAAGAAGAGTGCTGTTAAGGCCTCTCCATTAATCAACAGGTATG | | | Mouse | | | |
| 1893 | cg14807945 | CACTTGCCTTCCGAACAACTGCACAACAGGTGCAAATTAAAATGAAAACG | | | Mouse | | | |
| 1894 | cg01990488 | ATTTTTAGGGCCTCCTCCAGAGGCTCAGGCCCAAACAGATGTCCCTCTCG | | | Mouse | | | |
| 1895 | cg24648594 | GGGTAGCGGCAAACTTCAAAGCCATAAATCTTCCCTCTGACTGGCTGGCG | | | Mouse | | | |
| 1896 | cg23300110 | CCTGCCCCTCCCTACCTCACAGGCATGTTGTGAGAATAAATGAGGTAACG | | | Mouse | | | |
| 1897 | cg03082580 | CGGCTCTGTGGCACTCATACAGCTAGATAACCAAAGAGAGAAACGGGCCT | | | Mouse | | | |
| 1898 | cg05200513 | TTCAGTTGGGTGGGTGGAGCTGTTATGCAAACTCCCGCTCTCCTCATTCG | | | Mouse | | | |
| 1899 | cg05825073 | GGAAACCCGCAGCTAATGGCTCGGCAGTGATGCGCCAGGTGTGGGCCTCG | | | Mouse | | | |
| 1900 | cg00315292 | ATTAAGCGAGTCTGACAGCAGCATATTCAGGCTCGACAAGGGAACAGGCG | | | Mouse | | | |
| 1901 | cg25981029 | CGCCCCGGCTGGATTGGCCGAGCAAGCCTGGAAAATGGTAAATGATCATT | | | Mouse | | | |
| 1902 | cg09723377 | CGCCATCAGCCCCCTAGCCAGCCATCTGCTCCGAACCAAATGGCGTTTTT | | | Mouse | | | |
| 1903 | cg00809149 | CAGCCTGTCACCTTCAGGCAAAATTCAAAAATTCTGTTTTTATCAAAACG | | | Mouse | | | |
| 1904 | cg00863740 | CCTCTTCTGGCTGCTCAGCTGCTGCTGCTGCCGCTTACTGAAGTCCTGCG | | | Mouse | | | |
| 1905 | cg27026033 | CGGGCCCCAAATCCCCATGTGGTCCCGACATCATTGACATGGCGGAATCC | | | Mouse | | | |
| 1906 | cg22666763 | CGCGCAAATTGCAAAGTTGTGAATATGTTCTCAGATAATGAGCCTTCCCA | | | Mouse | | | |
| 1907 | cg21390110 | CGCTGGTGTGACAAATCAGATGAAGATGATTGGTCAAAACCACTCCCACC | | | Mouse | | | |
| 1908 | cg19272508 | CCAGGAGGTGCCCGCGGGCGGTGGAGGGCTGCTGCAGGTGCGTCTGCACG | | | Mouse | | | |
| 1909 | cg04845649 | CGCCAAGGAAGTGCTGCCCCAGTATTTCAAGCATAGCAACATGGCGAGCT | | | Mouse | | | |
| 1910 | cg01902998 | CGCGGACCCGCCCAGAGATATAAATAACATTATCTGAGGAGGGACATTCC | | | Mouse | | | |
| 1911 | cg21142636 | CGGGTATTGCTGGTGCTGCTGCACCTTCTGCTTGGACCCGCCAAGCAGGA | | | Mouse | | | |
| 1912 | cg18776124 | CGAAGCAGATTTCTTCACACTGGAGTCATTCACATCATGCCATCTCTTCG | | | Mouse | | | |
| 1913 | cg07991667 | TTCCTAGAGCAGGCCCAGCAGGTGAGCGGGCTCCTGGCGCAGCTGTGGCG | | | Mouse | | | |
| 1914 | cg19626252 | CAATCATTGGAACAAATAGCTAACATTTAACGTTTACACCCGCACTGGCG | | | Mouse | | | |
| 1915 | cg11735605 | CGTGTGTTTGTGCACTGCAGTTGGTGGTGGAAACTAAGGCAGTGGATCTG | | | Mouse | | | |
| 1916 | cg03201458 | CGACGGAGACCTTTGTGAAGGATATCAAGCCTGGGCTCAAGAATCTGAAC | | | Mouse | | | |
| 1917 | cg25649770 | CGTCCTCCAGATCGCTGAAAAGGAGCAGGAGCTGTTGGCCTCTCAAGAGA | | | Mouse | | | |
| 1918 | cg03182693 | GTTCCTTCACAGAGGCCAAGATGCTGGCGGTCGACTCCACCTCTGAGTCG | | | Mouse | | | |
| 1919 | cg04307172 | CGGCCCGGGAACCCTGGGCTGCTGGCGCCTGCGCAGAGCCCTCTGTCCCA | | | Mouse | | | |
| 1920 | cg12374442 | CGTCTTTTTGACGCAGGGAGAAATGGCAAATTTTAAGTACGTCATTAATA | | | Mouse | | | |
| 1921 | cg11741231 | CGTGATTACAATGGCTTATTTCATGGAATATTAAGTTCAAATTTTGCTCC | | | Mouse | | | |
| 1922 | cg13071028 | CGGCCAGAGAAGTTCAAACAATCTGGAAACTGGAGGTAACAGGTGATTTA | | | Mouse | | | |
| 1923 | cg17284641 | CGGCATTGATCGTCCCTGAGCTCATTATGTCACAGCTGGGGAACAGCTGG | | | Mouse | | | |
| 1924 | cg04765298 | CGGGGAGTTCTCCTGGCCCAAGAATGTGAAGACCAACTATGTTGTTTGGG | | | Mouse | | | |
| 1925 | cg13273551 | CGCGCTGGAAGTGGGAGCTGATGAGTGAAAAGAAAAAAAAGTCTTTGGAT | | | Mouse | | | |
| 1926 | cg21818536 | AACTTTTTGTTTTCTCCTTTCTTTGAAGGTTGTCAGACGTTACCGAGTCG | | | Mouse | | | |
| 1927 | cg13993270 | TATCCTTTTGATGGCTATAATTTATTCCAAATCGTCCCTGGACTGAAACG | | | Mouse | | | |
| 1928 | cg22978368 | TCCCTTTTCATTAATAATGAAAACCAGGGGGCAATAAAATGTTCTTAACG | | | Mouse | | | |
| 1929 | cg22670250 | GCTTTGGTATGTAATTATGCAAAACACTTTCCATAAAGACTTGCCATTCG | | | Mouse | | | |
| 1930 | cg27650171 | CTCAAAGTCCACTTGCGACAGGAACTGGTTATACAGGACACCTGGGGTCG | | | Mouse | | | |
| 1931 | cg06489241 | CGACTCCAAGATGTCAGTCTCCATGAGGTCACACTCCATGCTACTGTGGT | | | Mouse | | | |
| 1932 | cg14919009 | CATTTTTGTTTTTCTATCAGCTGAAATCGCTCTAGCATTTGCTCTACGCG | | | Mouse | | | |
| 1933 | cg14329189 | CGGCCTCTCCAAACTGATTGATTAGTCATGATCCCCGCAGTTTTAACAGG | | | Mouse | | | |
| 1934 | cg00549985 | TTTAAAACGCCCAACGCTGCCAAGTCAGAAAAGCGTCTCCTGATAAAGCG | | | Mouse | | | |
| 1935 | cg06889609 | GTGTAATATGGCGAAGACCTACGATTACCTGTTCAAGCTGCTGCTGATCG | | | Mouse | | | |
| 1936 | cg23534990 | CTTTAACATTTGGGATGCTGCAAATGCCGAAGTTAAATGAAATACCTCCG | | | Mouse | | | |
| 1937 | cg15258337 | GCTTGTCAGTGTGATGAAGATTGGCACCCAGGTAAGCTGTCACTCAACCG | | | Mouse | | | |
| 1938 | cg01656588 | CGGGCCAGACAACTCTATTATTGGCTTCATAATTGCTTAATTAAACTTTT | | | Mouse | | | |
| 1939 | cg00907438 | CGTCAAATTGCCTCCCAGGGTGCTGCAGTGGAGCGCACTGATTCGTACCC | | | Mouse | | | |
| 1940 | cg15708373 | CTGGTGATGTAACACCCATGTCAATACTTCCCAGTGAACGCACGTGTTCG | | | Mouse | | | |
| 1941 | cg02529685 | CGGAGATCATAGACACGGAAACTGTGCAACACAGGACCAACTCCTGCTAA | | | Mouse | | | |
| 1942 | cg07456629 | ACACAGGACCAACTCCTGCTAATGCTGAGGTATTTGGGAAAGCCTGATCG | | | Mouse | | | |
| 1943 | cg16113298 | TGGAGGATGATGACAGAGGTCAGGCTTCGCTAATGGGCCAGTGAGGAGCG | | | Mouse | | | |
| 1944 | cg20199880 | CCCGGGATTTGGTTGGAATTAATCTGCATCTCTAGCCAGGGTCTCATGCG | | | Mouse | | | |
| 1945 | cg12374136 | AGGTACAGGATGGTCCCTTTGAGCACTGCGTAGAATTTCTTCCAGCCACG | | | Mouse | | | |
| 1946 | cg16969806 | TGCTCACTCTGGTTGCCGCACTGGCCCAGCTGCAGGGTGATGATCTCCCG | | | Mouse | | | |
| 1947 | cg25716835 | CGCTGGCCTGGGCTAATAGTGCATGGTCGCTTGCTTGGATGTTCTGAGTG | | | Mouse | | | |
| 1948 | cg05434807 | CGGTTTCTGTAAAAGCATCGTTAGGAAGTGGGAACCATGCCTTTAATTCT | | | Mouse | | | |
| 1949 | cg09710669 | TCTCTCATCTCGCTCGGCTGCGGGAAATCGGGCTGAAGCGACTGAGTCCG | | | Mouse | | | |
| 1950 | cg05513929 | TTCCCGCCAATTATTATGGTTGCTTGGCTATTGAAGATTGTGAGCTGACG | | | Mouse | | | |
| 1951 | cg10004854 | CGGGCCCCGGTGTCCCCGGCCAGCACTATGCCCATCTTACTGTTCCTGAT | | | Mouse | | | |
| 1952 | cg06382378 | CGTGATCTGATCCCTAAGTTGAGACCTGTCTTCCTGTAACGCTTCCGAAT | | | Mouse | | | |
| 1953 | cg06198114 | AATTTGATTACTATAACCATACTTTTTAACTGCTGTCAGGAAAGACAGCG | | | Mouse | | | |
| 1954 | cg10429476 | CGGCAGGTGGAGGATTACCCTGTGGACATCTACTACTTGATGGACCTGTC | | | Mouse | | | |
| 1955 | cg06795598 | GAACGTGGACTAGACCCTGACACACTACCATCTCCTTCCTCCAAGGCTCG | | | Mouse | | | |
| 1956 | cg04784672 | CGACTGTGAAAGCCACCTGGAGCCACCTTGCCGGGATTGTACCTGCAGGC | | | Mouse | | | |
| 1957 | cg07479336 | GCTGCCTTGGCAGAATTCAGCTCTATGAATCACTTCGGAAAATTGGTTCG | | | Mouse | | | |
| 1958 | cg19351577 | CTGTGATCCATTCATTTCTCGCTCTACCTCCTGTTGCATAAAATGATGCG | | | Mouse | | | |
| 1959 | cg11286097 | CGGCCGTGCTCAACAACGCCTTCTACCTGGAGAACCTGCACTTCACCATC | | | Mouse | | | |
| 1960 | cg07679131 | TTTGGCTCGCCTCACCGGCAGTAATTTAATTTTTCCTTCATTCTGACTCG | | | Mouse | | | |
| 1961 | cg18808650 | CGGCCTCCTATGGTCATGCTGTAGAGACAGGAGATATGCTTGTTCTTGTT | | | Mouse | | | |
| 1962 | cg08030302 | CGGCTGTTGATCAAAGTGTTTTCTGGAATGGAAATATTGATGACAGGAGA | | | Mouse | | | |
| 1963 | cg19296063 | CGAGGCCGTTCACAGTACGACGAACGTGTTTCCGCTGGGGAACCTTCACA | | | Mouse | | | |
| 1964 | cg08921975 | GTTACAAAACCAAACTCTCACCACCTCCTTCTCCAGCTGTAAGCTGTCCG | | | Mouse | | | |
| 1965 | cg09060651 | AGGGCGGCATGCTAATGTGGAAATTTAAAGAGCCGCTCGGCTTTAAATCG | | | Mouse | | | |
| 1966 | cg09084199 | GATCGAAGTCATTAACATATTCCATGGCCAGGGGACTGGTGGGCAGGTCG | | | Mouse | | | |
| 1967 | cg12149905 | CGCCGGCTTTAAAGTCTCTGCCAGGATCCATGCTCACATGTTACTTCCTG | | | Mouse | | | |
| 1968 | cg09200936 | CGGCTGCAGATATATGCAATATTAAAGAGCGCTTATGAATTTTTGACATC | | | Mouse | | | |
| 1969 | cg09428649 | CGCCACTTCAGCCTGAAACTCCACATGCGTTGCCATCAGCACTTCCTGAG | | | Mouse | | | |
| 1970 | cg20375718 | AGCAGAGCCTGGTGAATATGACATCCCGCCTGCGACACCTGGCAGAGACG | | | Mouse | | | |
| 1971 | cg20718192 | CGGCAAATCAGGGGGGGGGAACTGTCAGTTGCAACCAAAGCCTGCCTGGC | | | Mouse | | | |
| 1972 | cg16861669 | CGTGAAGCAGCAGCAGCTGCCACAGCAGGCAAGCAGATGTATGAATATAG | | | Mouse | | | |
| 1973 | cg11102343 | TGCCTGTGGCGCTCCCAGGAAACACCTTTTTACATAAGATATTGCCCACG | | | Mouse | | | |
| 1974 | cg13684852 | ACAATTTAGATTACTTCATCTCTGCTAGCAACAAACTGAATGAAATTTCG | | | Mouse | | | |
| 1975 | cg13170453 | ACACATCAAACAAATACTTCTGTCTCCGGAGAGGAGTGTGCGAGAGATCG | | | Mouse | | | |
| 1976 | cg09772534 | CGGTTTCCAGGAAAGAAGAGAGAGGCGGAAAAGTTAACTTTCCGCCACCC | | | Mouse | | | |
| 1977 | cg11667953 | CGGCTTGAAGCTGAAAGGTGGAGTGTGTTTCTTTATTGAACACTGCATTC | | | Mouse | | | |
| 1978 | cg19926979 | CGAGGGCTTCATAAGCCTTTGATACAGTCTGATCTTTGAAACCTTTCCAA | | | Mouse | | | |
| 1979 | cg18538769 | TTGGTGGCAGTGTCAGAGTGACGTGGTAGGTCTCCATGGAGACGGCAGCG | | | Mouse | | | |
| 1980 | cg20377527 | GGGAAATTTTCTTCATTTTAATGTTCTTTCACAACCCACATTCAGTTTCG | | | Mouse | | | |
| 1981 | cg18881568 | CGATCCTCAATCAGTGGGTGGAGGATAACAGGGTAGATTCACTTGTGTGC | | | Mouse | | | |
| 1982 | cg26131171 | CGGAGATGTAGAAGATATTACAAGCTACTGGATGCACTGTCAGATTAACT | | | Mouse | | | |
| 1983 | cg18688640 | ACAGCCCTCTCCAAAACACATCATAAAACTAAGACTGAGATGAAGCATCG | | | Mouse | | | |
| 1984 | cg00658982 | CGTCATCTATGGGTTCTGGTAGTCCTGGGAGAGGCAGGGAAATGTCCTCG | | | Mouse | | | |
| 1985 | cg06370256 | CGGCCACGAAGAGGATTTGGAGGCGCCGCAAGTCAATATTTGATCACAAA | | | Mouse | | | |
| 1986 | cg01376725 | ACAGCCTTCCATGGTAAGGCCTGCGTGGAGCTTTCATTGCACCCAGTACG | | | Mouse | | | |
| 1987 | cg14830088 | GAATTAATGCCAGACATAGTTTTCCTGAACTCTATCCAACCGCCTAAGCG | | | Mouse | | | |
| 1988 | cg23815914 | CGGTTATGTGTTACAATGTGTTATGTGATAAATACGCTACCCGGCAGCTA | | | Mouse | | | |
| 1989 | cg02258144 | CGAGAGCTTCCTGAGAAGATGTCTGAATTTTTATGGCCTTCTCATTCAGC | | | Mouse | | | |
| 1990 | cg15985773 | CGGAGAAAATCAAGGCGGAGATCACAGTGGGGTAAAGGAATTATTAAGAA | | | Mouse | | | |
| 1991 | cg09635067 | CCTCCCACCGTGGACTTGTAGTGTTTGCTGAAGCTGTCCTGGGAATATCG | | | Mouse | | | |
| 1992 | cg16611537 | CGGCGGCCAAAGCCATGAACCGTCTTCTGTTGTTTGCAGAGTTGATCTTG | | | Mouse | | | |
| 1993 | cg12041712 | CACCTGGGGCAGTGGGTCTCCAGCTTTACACCAATGCCCTAGGGGATGCG | | | Mouse | | | |
| 1994 | cg16863579 | CGGCCAACTAACAGGTCAGATGTTCAAGTATAGACGAGTCATCTTGCCCA | | | Mouse | | | |
| 1995 | cg26639842 | CGGCAGCAAATTGGACCGGTTGCCATGGCAAATCTGAGCCCTTAAGTCAT | | | Mouse | | | |
| 1996 | cg04613777 | CCTCCTTCTTAAGAAGACTTTCCCAGGTCCCTCTTGGCTGTGGTTGAACG | | | Mouse | | | |
| 1997 | cg24378708 | GTCCTTTCAGGTTCAGGCGCAGCTGCAGCTGGACAGCGCTGTGGCGCACG | | | Mouse | | | |
| 1998 | cg20369280 | CGCAGCAGCACCGCCATTTGTCAAAGTCAACCTTCTGATTGCTTTGGACC | | | Mouse | | | |
| 1999 | cg05030744 | GCTCCTCGGGACAGTTCCCACACTGCAGTGCCTCTGGTGAGGAGACAGCG | | | Mouse | | | |
| 2000 | cg04839527 | TCACCAATAAAATTGGTCTCAGCATGGAGCGTAATTGCAACAAAGTGCCG | | | Mouse | | | |
| 2001 | cg05052045 | AATATCCCTCATTAATGCCACTTTATATTTACAGTGTGTCACTGGAGACG | | | Mouse | | | |
| 2002 | cg01824230 | CGGATATCGGGCACCTCCATGGCCTTCAAAAACATTGCCTCCAAAATAGC | | | Mouse | | | |
| 2003 | cg05165194 | GCTGCTCGCTCCATGATGAGATGCTAAATGAAAAGCTGGTGCGACACACG | | | Mouse | | | |
| 2004 | cg18404903 | CGGCGTCCCACCAGTCTTTTGGGGGGCTGATGTACATGCACCACAGCTCC | | | Mouse | | | |
| 2005 | cg18512092 | CAATAGGCGAGAGACTTCTTGTTTCCTGGCAGAGCGGGGTCCCGGCACCG | | | Mouse | | | |
| 2006 | cg18539981 | CATCATTGGATCCTTCTTTGTTCTCAACCTAGTCCTGGGAGTGCTTTCCG | | | Mouse | | | |
| 2007 | cg18552533 | AGAGGACAGCCTTTGGAGAGCTGCCCTGTCTGGGGACTGGCCCCTCCCCG | | | Mouse | | | |
| 2008 | cg11602744 | TGACGCGGACTAAATCATTTTCATTTAATTTCCGGTAAACAAAACCCACG | | | Mouse | | | |
| 2009 | cg15166827 | GTTACCCTGGGTGTATTTTGTCAGTCTGGTCATCTTTGGATCCTTTTTCG | | | Mouse | | | |
| 2010 | cg26098364 | ATGTAATCAGCGCCCATGGGGAGCCAGGAGCGGAGGGTGATGACATCACG | | | Mouse | | | |
| 2011 | cg16163981 | CGAGATGGACTTCTGGAAGGTGGACCTCAAGTTCCTGGACGACTGTTGCA | | | Mouse | | | |
| 2012 | cg19279211 | TACGTTGTTAGCAAGAATGCCCCATCACCACATCTTTCCAGAGCCTTTCG | | | Mouse | | | |
| 2013 | cg04817397 | CGGGTGTCTGACTCGAATGACAAGCGAGCTGATTTTTTCAGTACATAGCT | | | Mouse | | | |
| 2014 | cg12386526 | AATCTCTCATTCCTTGTTGTCAGCAATTCTCACCTAGTGCAGAGAATTCG | | | Mouse | | | |
| 2015 | cg25249359 | CCCTACAGACTCAATTAGATTCATTAATCTGCTGTGTAGACCTGTAAACG | | | Mouse | | | |
| 2016 | cg13141094 | CGGGGTAACCTTGACACAGTTGCGGCAGTCGCTAATGGCAGACACACACT | | | Mouse | | | |
| 2017 | cg16800641 | GTCTAACCAATATTAAAATGCGGCCACTTTTTAAAAAGCCACTTTAAACG | | | Mouse | | | |
| 2018 | cg07890174 | CGTTTCAAGAAATGCGGTTAACCTCGCAGTACTAAAACTGAATGAACAAG | | | Mouse | | | |
| 2019 | cg20588435 | TCACTTCATTTCGTTCAATTTCGTTCAATTTCATTCCTTTTCATCCAGCG | | | Mouse | | | |
| 2020 | cg00823476 | CGTACTTTGGCTGGCTACTCACCATCTTCAAGGTCGAGGTTTCTGGAAAA | | | Mouse | | | |
| 2021 | cg14192599 | TAGCCAGCCAAAGTACGATCTAATACGGGAGGTAGGCCGAGGTAGTTACG | | | Mouse | | | |
| 2022 | cg26466145 | CGGGGCTTGATGTCTGCAAAATGTCGGAAACGCAGCCAAGAGCTGCTAAC | | | Mouse | | | |
| 2023 | cg10918171 | ATTAAGGCAGCCTGTTATTGTGCGAGGCTGAATCAGTGGCTCTTTGTGCG | | | Mouse | | | |
| 2024 | cg06923126 | CGCATGCGGGGCGGAAAAGATTGTATTTATCACCTGTCAGGATCTGCCAG | | | Mouse | | | |
| 2025 | cg19962757 | CGCAGTCTGAAAAGCCTTCGACCTAAAGCAAAAAGGTCCCCAAGTGCCAC | | | Mouse | | | |
| 2026 | cg22979709 | CGGATCTCCCCAGGTTCCAGAGGATTGGGCCCACAGGGGTCATGCTCAGT | | | Mouse | | | |
| 2027 | cg16825428 | CGTGCGTTCACTGGGAAGTATTGACATGGGTGTTACATCACCAGCAGCCA | | | Mouse | | | |
| 2028 | cg10450039 | CGGCCTGTACAAAATGCAATGAATCAGGTTGGGATTGTGCTGAACGTACA | | | Mouse | | | |
| 2029 | cg21066109 | ATATAGCAGCTTGCTGTTGATTACCAGGGAAATGAACTCGCTGTCTGGCG | | | Mouse | | | |
| 2030 | cg21114170 | CGCTCATCTCTCACGACAATGAAGACCTCCTAGCGTCCAGGTTTCCGGCC | | | Mouse | | | |
| 2031 | cg23632380 | CGGGCAGCATCGATCGTGGCTCCTTTAAGACAAACTCAGACAGACATTTT | | | Mouse | | | |
| 2032 | cg10537936 | TCCATCTTCTGCCGCATCATGAGGTAGGCGATAACTAGCGTTGGGGAGCG | | | Mouse | | | |
| 2033 | cg04318752 | CCACGTTCTTATCATCTCAATTGCCGATTTTCATTTTGGTAACTTGGACG | | | Mouse | | | |
| 2034 | cg09408655 | ACTACATTAGGAATAAAACCTTCCACTTAAACACTTAAATGAAAACATCG | | | Mouse | | | |
| 2035 | cg12076803 | AGGCTGTTTGCAACAACCACTTCAACTCTATTTACAGAAGGGTTCCCACG | | | Mouse | | | |
| 2036 | cg12881209 | TAAACCACAGAGTCAAATAGCTCTAATAGAGATGAAGCCTGATAGATCCG | | | Mouse | | | |
| 2037 | cg21809124 | GACACGTAAAGAAGCAGCAGTCCTGCCCCAGTTCTCCTAAGAAAAATCCG | | | Mouse | | | |
| 2038 | cg18701907 | CGGCCATGCTCGAAGCCAGCTCTGCAGCTCAGCCAATCAGTGACATCATT | | | Mouse | | | |
| 2039 | cg26409285 | CGGGGTGAAAGTCGGCTTCCAGGCGTTCGATCTCCAGCCTTCCAGCTCAC | | | Mouse | | | |
| 2040 | cg05198393 | TTGATGCCATTGTTTCTGCTACAGATGGTTCTTTTAAATACAATAACACG | | | Mouse | | | |
| 2041 | cg14140337 | CGGGGCTGGAAGAGCTTCCACGGGATCCTCAAGGGCATGATCCTCTACCT | | | Mouse | | | |
| 2042 | cg17095319 | CGCGGAATATGTACATTTTTTAGTCTTACTTACGCTTTCAGGGGGTTGTG | | | Mouse | | | |
| 2043 | cg14119482 | TGGTGCATGTGGCTTTGAACTGGAGCCGTTCGCGTTTCCTGCAGAGAGCG | | | Mouse | | | |
| 2044 | cg22956116 | GGAAATGAATGACAGCTTACCTGATGCCAATCTTCATCAGACTGACACCG | | | Mouse | | | |
| 2045 | cg14497036 | ATCTGCCGCCAAGTTTCCAGGGAGTGACAGGGGACAGCAGGTACAGATCG | | | Mouse | | | |
| 2046 | cg06363348 | TGATGGACACGGAGCTTGCTGCAGCACCTATGACGCCAGAAATCTTGTCG | | | Mouse | | | |
| 2047 | cg24217623 | CGCGGCTCATAATTGTTTTTCACACATAAGTTATGCAAATGAGCTTTTAT | | | Mouse | | | |
| 2048 | cg23419136 | CGGCCGATCAGTGAATGGTCACCCCAGTCTGTCCAGTACAGGTACCTGGC | | | Mouse | | | |
| 2049 | cg27537290 | CATACTGCAAGAATATTTATTGAGCACAAGCTCAATCACCGCATTGAACG | | | Mouse | | | |
| 2050 | cg00833227 | CGGCAACAGTCCCCCCAGAACAGGCGGAGTGTGTGTCTGCCATTAGCGAC | | | Mouse | | | |
| 2051 | cg00570985 | CGTGCCCCAGGACTCAGAGAGCCAGCTGCAGGAGCAGGAGAAGCGGATTG | | | Mouse | | | |
| 2052 | cg23446200 | CACTCGCACTTCGCATTTTCTAATTACACCATCTGTGTCAGACTCTTTCG | | | Mouse | | | |
| 2053 | cg24429861 | CCGGTGTCTGTGCTATTGCTATGGGAACTGGCCATCCTGGGGGGGGGCCG | | | Mouse | | | |
| 2054 | cg26976071 | AATTCAGGTTAGTGGAGGCCGAGGATCAAAGCGCTCACCTCACGCTATCG | | | Mouse | | | |
| 2055 | cg24653257 | CCTGTTTATATTAGCGCGGTGTAAAATATTCTCGCTGTCTTGGGGAATCG | | | Mouse | | | |
| 2056 | cg27332443 | CTTTGTTAGCACAGCTCAGTTCAAATGTCTGATTAGTCCTTAGGAGATCG | | | Mouse | | | |
| 2057 | cg21279088 | ACTTTCCTACTTACCAAACCCGTCGCCATTACCAAGTCTTTCAAGCTTCG | | | Mouse | | | |
| 2058 | cg05522906 | CGAGTTTGCACTTCTGTTTACAAAGCAATCCTGCCATCAAAAGAGGAACA | | | Mouse | | | |
| 2059 | cg09415540 | CGATGGGATAATGAGACAGATGTCTCTCAACTGGAAGGACATTTTGACAT | | | Mouse | | | |
| 2060 | cg05163400 | TAAGTGCACGCTTCCCCCACTATATAGCAGCAGACTGTGTTGCCCCTACG | | | Mouse | | | |
| 2061 | cg25670483 | CTGCTTTTTGGTCCATGTTTAAAAAGCACCAGTGACCTTTTTTTATTCCG | | | Mouse | | | |
| 2062 | cg25879860 | CGTGCACAGCTTTGGAACTTTTGGCTATTCGGTATTTGTGAGTTTTGGAA | | | Mouse | | | |
| 2063 | cg25998250 | AGTGTTCAAATCGACATCATGGAGTCCATTTCTCCTTCGACTAGCATTCG | | | Mouse | | | |
| 2064 | cg08187246 | TCACCATGGAGCCCAGGATGGCCTGCAGCAGCAGCAGCACGATGCCCTCG | | | Mouse | | | |
| 2065 | cg26234638 | CGTGCACCTGGCTTGTCCCCGAGCGTAATATTCTTCAATGGAAACGGATC | | | Mouse | | | |
| 2066 | cg01149078 | GGAGAAGTTCACATTCCGGATTCTGCTCCGCTGCCCAAGGTGCAATTCCG | | | Mouse | | | |
| 2067 | cg06160165 | CGGACAGATCGATTACTCAGACACAAACGGATGTGCCAAGGGTGCCAGTC | | | Mouse | | | |
| 2068 | cg04671066 | TTGAATTTTCTTCACAATTTGTAGACAATTTGATGGATTAGGTCCCCGCG | | | Mouse | | | |
| 2069 | cg09816441 | AGCCTTAATATCTTACTTTGCAAATATAGTTTATTCCTTCAATAAAGCCG | | | Mouse | | | |
| 2070 | cg10651829 | AAGCCCCATTGCCCTCTTCTTTCTAAGCTGTCATCCTCCTGCTGCAATCG | | | Mouse | | | |
| 2071 | cg00414520 | GCACGTCCGTTACAAATAGGTAGTTTTGTTTCTCTTGTTGTCGCTACACG | | | Mouse | | | |
| 2072 | cg07289766 | ATTTGCATTTCTCCCTCATGCAACGCCCCTTTATGTAATCCATACAAACG | | | Mouse | | | |
| 2073 | cg01932632 | CGCCTGCTCCTCGTTGATGGGCTGGTTGTACAGCCGCAGGATCTCCTCCA | | | Mouse | | | |
| 2074 | cg19933464 | CGGTGGCACCAGCTCCTGCACAGGCCACAATGAACAGGTGATAGGACATG | | | Mouse | | | |
| 2075 | cg13752936 | CGGGGTGAATTTAATTGCTTTAGTTAAGTGGAAATCGGGTTAAAAATGAT | | | Mouse | | | |
| 2076 | cg14678895 | GACCTGTCGTGCCCTGCATGTTCAGAGTCTGTCATGTTTGGTCAAGAACG | | | Mouse | | | |
| 2077 | cg21244114 | GACAACAGCGATGAGGATGTGGTGCAGCACCAGGACCATGCTCGCAGCCG | | | Mouse | | | |
| 2078 | cg15026078 | CCGCGGCTATGCCCAGTCCTGCCGCTTCAAGAGGGTGCAGCAGAGACACG | | | Mouse | | | |
| 2079 | cg12285520 | CGGGCGATCCTCCGACTGCTCGGCGCCAACCACAACATGCTACCCACCGT | | | Mouse | | | |
| 2080 | cg18429525 | CGCGTGGCAGTGTGGATCCTGGAGAAGTATTACCATGACTTCCCTGTCTA | | | Mouse | | | |
| 2081 | cg22502858 | TTGCCCGTGCTGTACCTGTTCTGTGGATGAACAGAGGACTTCAGTCTGCG | | | Mouse | | | |
| 2082 | cg10221809 | ATCAGGGCCTTATTCATTAGCGCATAAACAATTTTGTTTCTCGGCACTCG | | | Mouse | | | |
| 2083 | cg04008656 | CGCACCCACCATGATGGAAGACGACGGGCAGCCCCGGACTCTGTGAGTAC | | | Mouse | | | |
| 2084 | cg04197976 | TTTTCAATACAACCAGATCATTGCAGATAATTGTATTACACAGTGTATCG | | | Mouse | | | |
| 2085 | cg25602724 | ATCGGTCATTCATAATTAACTAGTGATAATGTTCTACACTAATTTGTCCG | | | Mouse | | | |
| 2086 | cg09563729 | TGCTTACACCTGACTAAATCATCAGGCTGAAATCGGAGCCGACTCCCACG | | | Mouse | | | |
| 2087 | cg20655014 | TGATTAAAATAATTACAAATTGCACCCTGACAAGCAAGAGCAAAATTTCG | | | Mouse | | | |
| 2088 | cg14834925 | AGCTACACTGGTTGATTATTGCAGGAATGAAAGGCACTCTGACCTTGTCG | | | Mouse | | | |
| 2089 | cg05703789 | CGCCTGCTCCTGTTATCAATAGCAAATATGGACTCAGAGAAACAGATAAA | | | Mouse | | | |
| 2090 | cg14252486 | CGGGAGATAAGACACCTAATTGAGAAAACATTTGCAGTGGCATGTGGGGC | | | Mouse | | | |
| 2091 | cg23541245 | CATATTTCTCGCCGGATCGATACGTTTGACTCCCGACAAGACAATAATCG | | | Mouse | | | |
| 2092 | cg05109804 | AGCGTTACCTCTCTAGCTTTTTGTGCGGCAAGCTCAGCTTGTCTCTGTCG | | | Mouse | | | |
| 2093 | cg06537244 | CGGGTGAGAGTTCAGAGTTCAGCAGCAGCAGCCCGAGCCCATGATTCCCA | | | Mouse | | | |
| 2094 | cg00116110 | CGAGTTCATGTGCCAGCCACATTCAGAACAGGGTGTTCTGTGCTCTTCAA | | | Mouse | | | |
| 2095 | cg26257324 | CGGTATTCATTTCTGTGTATCCCTGCCAAAGATCCATTGTGTAATGTTGT | | | Mouse | | | |
| 2096 | cg00773632 | ATTAATCCTAGATGTACAATTCCATTAATAAGGCCGCTTTTTAATAGGCG | | | Mouse | | | |
| 2097 | cg02192891 | CGGCCGACGCTGAGATCCATGCCATTGTAGCCGTAGCCGTACCTGCCGGA | | | Mouse | | | |
| 2098 | cg01309929 | CGGCTCAAGGTCATCTATGGCTCCAGTGCTGGCTTCCATGCTGTGGATGT | | | Mouse | | | |
| 2099 | cg01351677 | CGCCTCATTTAGCTGTTTATTGCGCCTGAGTACGTGTTGCAGCTGTCATC | | | Mouse | | | |
| 2100 | cg18258323 | CCAAAGTATATGTGACCTAAAAAGGCATTGCTCATTCTGACACATATACG | | | Mouse | | | |
| 2101 | cg03536049 | CGGGTGACTTCCCAGCTGCAGAATCCTGGCTGCAATTAGAGACCTATACG | | | Mouse | | | |
| 2102 | cg08977423 | CTCCTTACTGTGGTACTAACCATCCTAGCGGATGGGGGATTTGTCCTACG | | | Mouse | | | |
| 2103 | cg04576672 | CGGAACTAGATGGTTATAGGGACGGCTCCAATCCAGGACAGACAAAATGA | | | Mouse | | | |
| 2104 | cg18010237 | CGGGATAAATTAGTGAAAGGGAAGAAAGAATAAGAAGAAAGGACTGAAAA | | | Mouse | | | |
| 2105 | cg18288070 | AACTCTCTTTTATTTCCAACAGCCCTAATCAGCCGCTCTCATTACAAACG | | | Mouse | | | |
| 2106 | cg10718356 | CGGTGGAGGGAAAGGGGAAAGGAGCCATTTTCTGCTGCACATCAGTCAGT | | | Mouse | | | |
| 2107 | cg06454821 | TAATTACTCGATTAAAACCTATTACACTTATTAGGGGCCGCTATTGATCG | | | Mouse | | | |
| 2108 | cg03445175 | TAGGGCTTGTGCACAGCGCCGTCATGTGAGCGCACGTGGTAGCTCATGCG | | | Mouse | | | |
| 2109 | cg11681358 | TCCTGTTATTCATAAGCGAGTTTCCTCTCTCACAAAGAACCTTTTGCACG | | | Mouse | | | |
| 2110 | cg06763662 | CGGCAAATGAAGTAATCTGCAAAGTCTGTGAACTCATTTGCATAATTGAT | | | Mouse | | | |
| 2111 | cg07699102 | CGCCCCTTAAGAATTGTAATATTTCTGTGTGGTAATGATAAACATTAGTG | | | Mouse | | | |
| 2112 | cg09164670 | CGCGTCACCACATGTCCAAAATATTCATTGCATCAATTCAAGGGCAGTGA | | | Mouse | | | |
| 2113 | cg17807911 | ATCACCGCGGTTTATTTAAGGAGCCAGCCCTCGTCTTTGATAAACTATCG | | | Mouse | | | |
| 2114 | cg14516881 | CGTCTCTATAAGCAGTGTTTTAGCTTAAGTGGCAGTTGCATATTAATTAC | | | Mouse | | | |
| 2115 | cg15485474 | CGTGCCAAATCTGACAGTGATAAAATCTCAAATGGCTCTAGCATCAACTG | | | Mouse | | | |
| 2116 | cg02227188 | CGGGCCACCTCATACCGACGGTCCCTGGTTAAATACATATTGAAGAGAAA | | | Mouse | | | |
| 2117 | cg03590800 | CGTTCCCACGTCATACATAGATGTAACTCTAGAGAAAAACAGTAAAGGTG | | | Mouse | | | |
| 2118 | cg17641835 | CGGGAATTTCACTTGCTGTGCAGATCCCGCTGTCCGGCTGTCAGACACCC | | | Mouse | | | |
| 2119 | cg18033250 | TGTTTACTTCCTCCAGAAACAAGAGCATCATCAGCACGCTCAGTGCATCG | | | Mouse | | | |
| 2120 | cg23120132 | CGGGGAAGAAGGACCTTGTAATAAATAATATTCTGCACATCAAATCACTT | | | Mouse | | | |
| 2121 | cg09233844 | CGCACGATGACGTCAAACGCTGCTATGGCAACCGTTGATTGGTGCCCACA | | | Mouse | | | |
| 2122 | cg05857652 | CGGGGGTCAAAAAATCGCTTCAGGCTGCAACTTTACCTTAGAGACGTTTT | | | Mouse | | | |
| 2123 | cg07234865 | CGGGGTTGGTTGTTATCTTTGGTTATCTAGCTGTATGAGTGGTGTGGAGT | | | Mouse | | | |
| 2124 | cg13832100 | CGGATGGAAATGCCCATAAAGCCGTATCAAACCCTGTGCAGAGAAGGACA | | | Mouse | | | |
| 2125 | cg06149706 | TTGTCATTCAGTTTATCCAGTTTTGGTCACCCATTTTATTTATTTATGCG | | | Mouse | | | |
| 2126 | cg10068143 | GCCCAGAGGAGGCGAGATCACCCGCACTAATTTGTACATATCCTTATACG | | | Mouse | | | |
| 2127 | cg06558044 | GCGGCACTGGGAAACCCAGCAAAGCTAAAGCTATGCTGGAGGCGGGGACG | | | Mouse | | | |
| 2128 | cg18962324 | CGGCGGCGGCAGCTGCAGTGACATGTCCAGCATGAATCCCGAATAGTGAG | | | Mouse | | | |
| 2129 | cg07065655 | GCATCAAAGGGATGCAGCTTCTCCAGGCCCATGAAGGTGATGTTGTAGCG | | | Mouse | | | |
| 2130 | cg15265788 | CTCATTTGAGAAGAGCAGCATTTGAGGCAGCAGCGTTCGCCTGCTGAACG | | | Mouse | | | |
| 2131 | cg01456511 | CGCTCAGTTCCAAGCAGGTATTTGGCTGCGATTAAGTCTCCAATCGGGCT | | | Mouse | | | |
| 2132 | cg07938834 | CAGGTCAACTACAATGATGGCTCCCAGGAGGACCGAGGTGTGTGTGGCCG | | | Mouse | | | |
| 2133 | cg03504911 | CGGCGGAAGGAACACCAGCGACGCTTCTACTGGGCTATGATGTATGAATA | | | Mouse | | | |
| 2134 | cg21550097 | CCCAAAGTAAATAATACAGTCGGTATATAAATCTTTCCATTAAACTATCG | | | Mouse | | | |
| 2135 | cg15918741 | CGTGTTTCCGCTGGGGAACCTTCACACTGTTGGGGAAGATTTTTATAGTC | | | Mouse | | | |
| 2136 | cg13719093 | CGCATACATTCACAAAGTTCTCTGCGGCTGAGGTAGCGGAAGACAGACAT | | | Mouse | | | |
| 2137 | cg07810218 | TCGGCTGTTTTATAAAAGGACAAAACAGCAAGTGTCTGCATTGCATCTCG | | | Mouse | | | |
| 2138 | cg12835327 | CGGGTGGAGGTGCGCTTCTGACAAGCCCGAAAGTCATTTCCAATCTCAAG | | | Mouse | | | |
| 2139 | cg03308445 | CCGTCACGAACAGGATTCGAACCTGTGCGGGGAAACCCCATTGGATTTCG | | | Mouse | | | |
| 2140 | cg13544184 | CACTGAGGCCGTGGATCAGGGGGGCGCTGTTAGACCTCCTGAGGCCCCCG | | | Mouse | | | |
| 2141 | cg03475710 | ACTTTGCTGACAGCTTGTCACATTTCACTGACTGTCTGTCTGGGGTACCG | | | Mouse | | | |
| 2142 | cg18673669 | CAACTCCACAATTACCTTATATTTAATTAATATTTTTTCTCCTCTTTACG | | | Mouse | | | |
| 2143 | cg09711113 | AGAGGTTGAGGTACAGGCAGTCCTCGCTCTGGTTCTGCACGTAGGTGGCG | | | Mouse | | | |
| 2144 | cg20960545 | CTCCCCTTCAACAGGATGTCTCTCAGCTCGGGCGACATGAAGTAGGGTCG | | | Mouse | | | |
| 2145 | cg24798730 | CGAGTTCTTGGTGAAAATGCAATTGCTGTTCGAACAAAAGCCATGAAGTG | | | Mouse | | | |
| 2146 | cg10316601 | TGCATGAAGTATAATGCTAAAGACACAATTTTCCACCGAGCAGCTGTCCG | | | Mouse | | | |
| 2147 | cg21128693 | CGTGTGACAAAAACAGACACTTACTGCCGTAGGGAGAGTTAGCGGAGGAG | | | Mouse | | | |
| 2148 | cg09660031 | CGTCCATTTATCTGATCCAAGTGCTCTGAAAAACTGCCAACTGTACAAAG | | | Mouse | | | |
| 2149 | cg15416058 | GGCCTAAACACAATAGGACTCGCCATTTTCTCCTTGTGAAGCACAAAGCG | | | Mouse | | | |
| 2150 | cg21830760 | AATGTGCCTACCCATCGCTGTTGCTCCTTGCAGCCTGCGGGTCCTCTTCG | | | Mouse | | | |
| 2151 | cg21913180 | TGCTCAGCCTGTGCCTTGAACTCGGTGGTGCAGAGCGTGCTGAGCAAGCG | | | Mouse | | | |
| 2152 | cg07240567 | TGCGAGGAAGATGGGTTAATATTCTGTGCATTCTTTGCCACCTTCATTCG | | | Mouse | | | |
| 2153 | cg22359606 | CGCCATATGGTCTTCCCGGTCCAGCCAAGAGCCTGGAACCACGTGACCTG | | | Mouse | | | |
| 2154 | cg06075167 | CATCGATATCCACAGCTGCAACCACGAGGCTGAGAAGCTGGGCTGCAGCG | | | Mouse | | | |
| 2155 | cg12906435 | CGACTATTTAATGAGTCATTCAGCCAGGATGATTTTCTGTCTGAATATGT | | | Mouse | | | |
| 2156 | cg17696796 | CGTATCCCCCTGCGGAGACAGGTAACAGAAAGTAGATAAAGAGTTTAAAG | | | Mouse | | | |
| 2157 | cg16091716 | CGCCGAAATTGAATGCATCGTTTCTTAATAAATCCAGGCCTGGACTCAGA | | | Mouse | | | |
| 2158 | cg14705908 | ATACTGAGGGTTAGTGGACCGTGTTACATACGTCACAGATAAAGCGTACG | | | Mouse | | | |
| 2159 | cg14894602 | CGGCATCATGAAGCAGAATTAAAGTAGGCATCTGTGTTCTCCGGCATTAA | | | Mouse | | | |
| 2160 | cg21658772 | CGAGGTACCGAGGAAAACAAACAATGGATTGTTACAGTACATACATTCTG | | | Mouse | | | |
| 2161 | cg01190601 | CGAGGGAAGACAATCTGCTTCTCGCATTATTAGACACGCTTGGAGTTCAG | | | Mouse | | | |
| 2162 | cg26603448 | CGAAGTGTCTGCAATGAACCCCGTGAATGCTACTGCTCTCTACATTTCCG | | | Mouse | | | |
| 2163 | cg26572218 | CTAATTGAAGTGACGGCTTTGGAGATTATGGGGAACATATGGCCCTACCG | | | Mouse | | | |
| 2164 | cg16812753 | CGGGCAGATTTATTCCGCTGCCAAGCCTGCAGAAAATTGACTTGTTCATG | | | Mouse | | | |
| 2165 | cg23745624 | AGGAGGGCGAGACCCCCGGCAATTTCATGGAGGACGAGCAATGGCTGTCG | | | Mouse | | | |
| 2166 | cg24694549 | CGCACATACCAGGAGAAAGGTAGTCCCAGTGGGGAGTGACAAAGCTTAAT | | | Mouse | | | |
| 2167 | cg25836134 | CTTTGAATGCTTATAAAACTCCACATAAAATCGGACTGACCCAAAACACG | | | Mouse | | | |
| 2168 | cg19860820 | GTGAAAGAGGTCAACAGCGGTTTAACACTTTGGTTCAAGCAAGGCACTCG | | | Mouse | | | |
| 2169 | cg25295558 | CGCCTGAACCTGAAAGGACAAGGGCGTCACGTTGCAATGACTATCCTAGG | | | Mouse | | | |
| 2170 | cg27070413 | TTTAGGGGGTTGGCTCCTACTCTGCTAGGACTGTGTGCGCGGAGCATGCG | | | Mouse | | | |
| 2171 | cg24813264 | CCTTGCCGCAGATGGTGGTCTTGTAGTCGCCAAAGATCTGCCTGACTGCG | | | Mouse | | | |
| 2172 | cg06582198 | TGCGATGCTAATGGCAGGTGCCTCTTGGCAGCACTGACCATGGACAATCG | | | Mouse | | | |
| 2173 | cg27278067 | CACTAGCAGCCACAGCCTTCTCTCTGGGGTTGGGGTTCACTGGTTAGCCG | | | Mouse | | | |
| 2174 | cg01667686 | CGGGTGATGATCACGTCTATGAAGTTAGCTGCAGTAATGGTAGTCTTCCC | | | Mouse | | | |
| 2175 | cg21539484 | TATGATGAATTATGGAAATGACTGGGACATGTACTTGGTTCCCTCCTACG | | | Mouse | | | |
| 2176 | cg18298247 | GATCCACAAGCTCTTGCCAAGGCTGTACAGATTCACCAAGATACCTTACG | | | Mouse | | | |
| 2177 | cg02456288 | CGTTGGCCAAGCACAATTGTGTTATTGGAGATAATGAATTCAATCCCCAT | | | Mouse | | | |
| 2178 | cg16492924 | ACTCAGCATTTTGAAAGATACTATTTTTCACAAATCACAAATGGCTTTCG | | | Mouse | | | |
| 2179 | cg08191552 | TTCATGGCCATGTAGTGACCCAGCTTGGCGCTCTGGATGGTGACCACACG | | | Mouse | | | |
| 2180 | cg08122075 | TTTCACTTTTAATCAAGCTTCCTGTTGTAAGAGCTCAGCAGACAGTGACG | | | Mouse | | | |
| 2181 | cg27450153 | CGAGCCCACTGTTGGAATAGTTGCTGATGGGGGACTGTTGGCTGCTGCAT | | | Mouse | | | |
| 2182 | cg17026002 | TTCCTGACAACCTTCTACAGTTATTGACATTACAAATACCAGCAACAACG | | | Mouse | | | |
| 2183 | cg24479261 | CGGACCTAAGACCGGGAACACAAAACAGAGAAAAAAGGACAATTTTGTGG | | | Mouse | | | |
| 2184 | cg08121938 | CGCCAGCTGCAGGTCCAGGATGTAGTCGATAACGTGCTGCAGGATCTCCA | | | Mouse | | | |
| 2185 | cg09084634 | CGATGATCTTCATCTGCTGGTCGGAAGGGCTGTGGAATTGGAATGGATTT | | | Mouse | | | |
| 2186 | cg09349243 | TTCTCTGGGTTCCTCGGCCCACCATTCTTGTTCAGTCTTACCAGAAACCG | | | Mouse | | | |
| 2187 | cg05166684 | CGTGCAGACCCTGGTGCATCTCAAGTTCCCCACAAAGGTGGGCAAGGCCT | | | Mouse | | | |
| 2188 | cg18881373 | ATTGGACCGCAGCGCTGAGAGCTTTTGTGTTTAATGACCTTCAGCTACCG | | | Mouse | | | |
| 2189 | cg12185545 | GTTCCACTCCAGTGCCCTCCCCAATAAAACTTTTCATATCCTTATAAACG | | | Mouse | | | |
| 2190 | cg03178974 | CGGGATTATACCGCTCTGTGCAGGCCCACAGGAATCAAAGTCGGAGAGAA | | | Mouse | | | |
| 2191 | cg05010174 | GGACGTCCCAAAGACAGATATACTCATATGTAATCTGCAAGGCACATTCG | | | Mouse | | | |
| 2192 | cg06600911 | CAAGTATTTAAAACCTCTAGAAACCCATGATTTGACAATCAATGATTGCG | | | Mouse | | | |
| 2193 | cg22621796 | CGGCTGCCTTTAAATAGTCCGATCTATCAGAGGGGCACACGCTGCCCATA | | | Mouse | | | |
| 2194 | cg08097199 | CGGCCCTTGGAGACTTCCCGCTTCACCAAGCTGCTGCTCAAGCTGCCGGA | | | Mouse | | | |
| 2195 | cg19923477 | CGAGACCACTTGTAAAAGCAATAAAGCACAAGGAGTGACTGCAGTAAGTG | | | Mouse | | | |
| 2196 | cg08778230 | CGCCACCTTTTAATCTTTTGTAAGAGTGAAAAGGCAGAAAAGGAACGCTC | | | Mouse | | | |
| 2197 | cg12503970 | CGCTACAGCACTGGATTAGCTGGAAACCTCCTGGGAGACCTACATTACAC | | | Mouse | | | |
| 2198 | cg20380871 | TGATCTGCTTCCTGGAGAACTCCTTGAACTCGGTGTAGGGGTTGAAGACG | | | Mouse | | | |
| 2199 | cg21853871 | CGCATTAGCGGACACTCTATAATAGTGGCATTTATTAAGAGACCTGTTCT | | | Mouse | | | |
| 2200 | cg09137804 | CGGGGATGATCTGGGTTCCGGGGGAAGTGGAATTATTTTTTACCAGCGAA | | | Mouse | | | |
| 2201 | cg26339573 | TCTGTACAGTGTCGACAGTCTGTAATGCCTTTTTAGCATGAATGACAACG | | | Mouse | | | |
| 2202 | cg21164975 | CGGATGACCAAAGACCTTTCTTATGCCGGAAGCAAAAACCAAAACTTTTT | | | Mouse | | | |
| 2203 | cg23602865 | GGTCAGGAGAATGTACCATTTGTAAACACCCCTTTCCTTTTTTATTCACG | | | Mouse | | | |
| 2204 | cg18178230 | TGGAAGTGATGTGCAAGGCAAGAGATAACCAGGTAGAACTCAGCGATTCG | | | Mouse | | | |
| 2205 | cg26518080 | CTATCCGGAATATTTTGGATTCTTTGTCCTCCCATCGGATGAAGTTTTCG | | | Mouse | | | |
| 2206 | cg15136978 | CGTGGCCAGGAACCTCCGCATTCACATGACCAGTGAGAAGCACATGCATA | | | Mouse | | | |
| 2207 | cg27039984 | CGGCTCTATGACGATGCCATTGGTAGAATTATTTAGTTCTCTGGAGACAA | | | Mouse | | | |
| 2208 | cg21474259 | CGTCCCATTCAGCTTCTCCCGGGCTCGGTCAGCATCTGAGCTAGTTTCAA | | | Mouse | | | |
| 2209 | cg26640686 | GCATTTGTAACGAATTTCCGTTCGAGTTTGTATTTTAGGCGCCATTTTCG | | | Mouse | | | |
| 2210 | cg21472737 | CTCGTTCTCTCCTCCGTTGCAGATTGAAGTAGTCAATACTTTCAAGAGCG | | | Mouse | | | |
| 2211 | cg07117806 | CGCCCTAAATCACTGAGGCGGTCAGAGAACAAACTAGGTTTAGAGTCATC | | | Mouse | | | |
| 2212 | cg02496392 | GACTCTGTGACACGTGGAATAAAATCACGCTCCCATAATGGGATCATTCG | | | Mouse | | | |
| 2213 | cg11145593 | AATCTGAATCTTTTTTCTCCCCATAGTCCCCGTTTTTCTTCTCTCTCTCG | | | Mouse | | | |
| 2214 | cg10239342 | ACAGGTGGCTTCCAGACCTTCAACTTCAAAACAGTCCGTCAAGGTCAGCG | | | Mouse | | | |
| 2215 | cg16895245 | TCAAAAGAGTGCATAAATGAAACAATGTCCCCAACGGGGTCAGTGACCCG | | | Mouse | | | |
| 2216 | cg08981194 | GCTGTGCTGGTGATCACAAGAAACAACAGCAATCTGTTGAAGATCAGACG | | | Mouse | | | |
| 2217 | cg17247426 | TTAGTTGCGTTGAGTCATCAGGCAAAGTGAGTCCTTTTGGGTTGTCCTCG | | | Mouse | | | |
| 2218 | cg17625993 | CGGTGATACATTTTGTACAATAATTAGTTATTTGATTACTTTTGTTTTAG | | | Mouse | | | |
| 2219 | cg22723228 | GCTATTTTTGATGCAATGTTCTTAAAGGCAATAGATGTCCCAGATATTCG | | | Mouse | | | |
| 2220 | cg02376954 | ACCTTGATTCACTCTTCGTCGTTGGCTAGGTTTTGGATTTATTACTTTCG | | | | Rat | Human | Universal |
| 2221 | cg03942000 | CGGGCTGGCCAGGCAGCTGCAACCTGCGCTCAGGCAGCTCTTAAAGACAT | | | | Rat | Human | Universal |
| 2222 | cg25381383 | AGCAATTAAACAGTTTGATTAAATGAGCCATCATAACAATGATGTCTGCG | | | | Rat | Human | Universal |
| 2223 | cg04958293 | CGGCCTTGTGTCAGCAGGTCATGGTCCCTAGAGACTACCTAGTTGTAGTG | | | | Rat | Human | Universal |
| 2224 | cg16356803 | CGGTCGCAAATGGTTTGACTAAACGTAGGATGGGACTTAAGTTGAACGGC | | | | Rat | Human | Universal |
| 2225 | cg16486681 | CGTCAAGTTTACTCACGAGTTTACTCAGCACAAGGAGGGGGAAGAGGCCA | | | | Rat | Human | Universal |
| 2226 | cg27088374 | CATACCTTATAAACTCTTCTCCAATTCTTTTTGTTGTCTTTTAACATTCG | | | | Rat | Human | Universal |
| 2227 | cg18094982 | CGTGTGGCCTTAAACTTGCAGCAAATTGCAAAGAAATGGCTCAAAAGCTT | | | | Rat | Human | Universal |
| 2228 | cg14155184 | GTGCGTCTTCACGGTGGTCATGACCTACAGTATCACCTGCCCCATCATCG | | | | Rat | Human | Universal |
| 2229 | cg19591642 | CGTGGCCAGCCATCTCCTATTGAACCTGGAGGACTGGCTGGTGAAGTACT | | | | Rat | Human | Universal |
| 2230 | cg21815434 | ACTCCCTGCAGAAGACGTACTCGGTGTAGCTGGTCCAGATCTTGTCCTCG | | | | Rat | Human | Universal |
| 2231 | cg27299745 | ATTCCCTCACTCGACTTTTCCCTAATGAGTTCGCTAACTTTTATAATGCG | | | | Rat | Human | Universal |
| 2232 | cg00694357 | CGGTGTCCCAAACTAGGTCAGCAGCGCTCGTTTCCTGTGGGTCAGCTAGC | | | | Rat | Human | Universal |
| 2233 | cg00929113 | TCTCCTTCAACTAGACGCTTATTCATGTCGCCTTAATGAGAAACAAAACG | | | | Rat | Human | Universal |
| 2234 | cg01063757 | AGGATCTTCAGTGAGAGACATTATTCTTGGAATATCCAATTAATTCCACG | | | | Rat | Human | Universal |
| 2235 | cg02456804 | CGGAGAACCTGGAGATCTTGTTGGTGGGGCCAACCAGGGTCACAGTTTCT | | | | Rat | Human | Universal |
| 2236 | cg03222030 | GGTTGGCCGTTTGCTGCGTCTAGGGGTGTTCTATGTAGCGCAGCAGTTCG | | | | Rat | Human | Universal |
| 2237 | cg03521797 | CTAGGAGCCTAGGCGGCACTGTTCCTTTGTCCCCTCAGCTCATCTTCACG | | | | Rat | Human | Universal |
| 2238 | cg05008190 | ATGGGCGGGCAGGACAACACGGCATGGAGCAGCTCCACATGGACACCCCG | | | | Rat | Human | Universal |
| 2239 | cg09629523 | GCCCTGAGCTTTTCTGGGTGTGTGTGACAAAGGCTTAATCTGTCCGGACG | | | | Rat | Human | Universal |
| 2240 | cg10487127 | CTCCACGGGGCTTGTTTTGGATCCCCCTGCAGTGAAACAGGTCCTGTACG | | | | Rat | Human | Universal |
| 2241 | cg10644072 | GAGCGGCAGGGGCATGTGGATACTGGCTCTCTCCTTGTTCCAGAGCTTCG | | | | Rat | Human | Universal |
| 2242 | cg11541320 | CGTTAATGTAATCAGCAGTGTGCGAAATGACTGCGCGGAAAATAGCATCA | | | | Rat | Human | Universal |
| 2243 | cg11690941 | TGATGCTCAGTTGTCAGACTACCCTTCTTTGGACTACCAAGGCCTCTACG | | | | Rat | Human | Universal |
| 2244 | cg11803466 | CAGGCGGCCTCTTTTGTTTATCTGACAGCTAAATATCAAGGCAATCACCG | | | | Rat | Human | Universal |
| 2245 | cg11997199 | GGCAGCAAGCCCTACAAGTGTGACGAGTGCAGCTACACCAGTGTCTACCG | | | | Rat | Human | Universal |
| 2246 | cg15597031 | CGTGTTGCGCTTTCATCCAATTTTCTGATGTGTTTTGGTAATGATGTTGA | | | | Rat | Human | Universal |
| 2247 | cg16296826 | CGCTCCCCCTCTAATGTGTGATCTGGAAGCTCTATAAAGCCTGATGTAAT | | | | Rat | Human | Universal |
| 2248 | cg16391501 | CGCTGCGCAGCTGAGAGCCTCGGCTTAATCTTATGCACCATTAAGCACTC | | | | Rat | Human | Universal |
| 2249 | cg17425418 | GGGCTCATTGATGGCCTTACTAAGTTTTTTACACCATCACCTGATGGTCG | | | | Rat | Human | Universal |
| 2250 | cg19414994 | CGAGTGCCTTGAGCCTGAGCAATGTAGCAGGCGTCTTCTACATTCTGGTT | | | | Rat | Human | Universal |
| 2251 | cg19854621 | TTTTTCTTTCTAAGCTGAGCACGTAGTCTGTTCAGAGCTTGTTTCTTCCG | | | | Rat | Human | Universal |
| 2252 | cg20896439 | CGGTTGTCCTGCCATAAGTAGTAGAAAGTGCCCAGGATCCAGGCTACGGT | | | | Rat | Human | Universal |
| 2253 | cg22588813 | AGGTCTCTCTGTTTGGCTGGGGGTCCTCCTCCACTATCTGAGAATCCACG | | | | Rat | Human | Universal |
| 2254 | cg22866580 | CACAATAAGTCAAGGTCATAAAACAGTAATGTCAGGACGGTCTTGGTGCG | | | | Rat | Human | Universal |
| 2255 | cg23405931 | TGTGAAATGGAGCTGTGTATATTGAAATTTCTTTCTCCAAAAACAGAGCG | | | | Rat | Human | Universal |
| 2256 | cg25217825 | CGATCAATGGCTATCAGCAGTTCTTGTGAGAGGGCAGAATAATTTGATGG | | | | Rat | Human | Universal |
| 2257 | cg26120306 | CAAACTGGTTGTTAACTGTGATAACAACTGATTTCAAGGGGTTATTGCCG | | | | Rat | Human | Universal |
| 2258 | cg26845017 | CGCGGAAGGTCTCCGGCGAGGTCTCCATTACTAATAGAAGTTTTTTGATG | | | | Rat | Human | Universal |
| 2259 | cg27291552 | CGGCTGTGGGTCACATTCATGTGTTTCTCCAGGTACCAGCGGTTGTTGAA | | | | Rat | Human | Universal |
| 2260 | cg04243635 | CGTCTCATCGCAAAGTAAATAAATTATGCCTATCATTTTGGCAGCTTAAG | | | | Rat | Human | |
| 2261 | cg05775627 | TGCAGCTTCTTGAGGAACTCAATCTCATCCATCAGAGACTCAATCTTGCG | | | | Rat | Human | |
| 2262 | cg11134720 | GCTTGTTACCTGGTCATCAGTTCGAGACATCTCAGCAATGGGCACTACCG | | | | Rat | Human | |
| 2263 | cg02129856 | CGCGGGAATGGCAAATATACTATTTGTCTAATGGCTCGGCAATTAAATTC | | | | Rat | Human | |
| 2264 | cg25445920 | ATGGCTGGGGCTGCCTCTGTACAATGGGATAAAATTCAAGTTCAAGTGCG | | | | Rat | Human | |
| 2265 | cg16435898 | GACGGGTCCAGCATCCAGTAGTTGCCCTTGCCCGGGTCGTCGTAGTGGCG | | | | Rat | Human | |
| 2266 | cg08333796 | CTGGCTCCCGATTCGGGATCCAGTTTCAGAGAGCGAGAGATTGGGGAGCG | | | | Rat | Human | |
| 2267 | cg10187298 | CAGGATGGATGGGGGGACCGACAGCCGCGGAATCCCTAATAAAGGCCGCG | | | | Rat | Human | |
| 2268 | cg04276865 | CGCTGTCGTCTCTGTTCATATATATTTAGTATTTATTTACAAGGCTGGGT | | | | Rat | Human | |
| 2269 | cg14467787 | TGTCATTCATCAAAAAGTAGCGCCCGCTCGTCATTAAGGTACGAATGACG | | | | Rat | Human | |
| 2270 | cg09129254 | TCACAGCTCGGAGTGAATGGTGTTTCTGGGATACCACAGCAACCTTGACG | | | | Rat | Human | |
| 2271 | cg03751003 | CGCCAACAGCCTTAAAACTTGCCACAGTTGCTGCCAGTATGGACAGAGTG | | | | Rat | Human | |
| 2272 | cg14188130 | TACCTTCCTTCTCCTTCCCTACAATTAGGACAGGAACAGGCAACTCTTCG | | | | Rat | Human | |
| 2273 | Cg07082738 | CGCCCCTGCTCATCCATGCTAGCCTGGTATTGCAGCTGCAGCTCCCGCAG | | | | Rat | Human | |
| 2274 | cg20315951 | CCGGTTCGACAGGTAACAAATAGGTGGGTTGTCACCGTTATTTCCCAACG | | | | Rat | Human | |
| 2275 | cg27393099 | CGCCAGGCAGTGAAACTTAATAGCAGGACAAGAAAACGGTTTAATAAAGA | | | | Rat | Human | |
| 2276 | cg17063148 | CGCTGTAGACAGCGCTGGAAATAATCAGATTAAGGCCAATTATGCGTGAG | | | | Rat | Human | |
| 2277 | cg12322817 | CGAATTTAGCAAATCAGATCATGGTTTAAATGCACTATTTAAAGCAGTCC | | | | Rat | Human | |
| 2278 | cg26987152 | GCTGCTCCAGGAGCCATGGCTAATCATCTTATAAGCAATGCTCTGCTTCG | | | | Rat | Human | |
| 2279 | cg24765266 | TCTCTCTTAAGTGCCATCGAGGGGCGCCGGGAATTGCAGCCAGTAAATCG | | | | Rat | Human | |
| 2280 | cg20672483 | CGCCAGCAATAAGACCAACCGCTTTGCTATTGTCCAAGTGGAAAGAGCCA | | | | Rat | Human | |
| 2281 | cg13228949 | TGTCAAATCCCCCTTAATTACCAGAGATTTTATAGCAGCTTCATTATACG | | | | Rat | Human | |
| 2282 | cg14701026 | CAGACCATTTTCATTCAGGAAAATCAATAGACAACTTACTGTGATGTACG | | | | Rat | Human | |
| 2283 | cg16184756 | CTAATTATCTCGGGCAAGATGTAGGCTTCTATTGTCTTGTTGCTTTAGCG | | | | Rat | Human | |
| 2284 | cg08275784 | CGAGAAATTTTATGGCCATAAGGAGCGTACTCTAATGGTGATTTTTGAGG | | | | Rat | Human | |
| 2285 | cg25830596 | CGACATAAAGCGTATCAAATATTTATTTATCTGGGCAACAAAGGACTATG | | | | Rat | Human | |
| 2286 | cg18440245 | GATTTGTGGATAAGCCTGAAGCAAAGTCAATGCTCGGTGTCAATCCCACG | | | | Rat | Human | |
| 2287 | cg24203137 | CGGGGACCTAATTCCAGGCCCAGGATCGAATTTTCTGGCAACAAAGGGAA | | | | Rat | Human | |
| 2288 | cg22527167 | CAGCCAAGTAGACTTAAAGTAGTCACTTATTTCCCAAAACTGGTTTGTCG | | | | Rat | Human | |
| 2289 | cg07116247 | CATCACCTTTAACCGCTACAGCCATGATTTCAACTTCCACATCAACTACG | | | | Rat | Human | |
| 2290 | cg26451479 | ACAGGAGCACCTCCAGCACGCAGCAGGGGAACTTCATCAAAGTCAGGACG | | | | Rat | Human | |
| 2291 | cg05927178 | TCAAAATGCAGGTTGGAGTTTTGACATTCCTGCAGGCTCTGAACGCCTCG | | | | Rat | Human | |
| 2292 | cg00393367 | CGCCTGGGAACTGCAGCAAATGGGAGAAGTATGCATGGATTTCACAGGTA | | | | Rat | Human | |
| 2293 | cg17466139 | CGCCTACAGTTTCGTTTCCATTCACGACGCGTTCACTGATCGCCACGAGT | | | | Rat | Human | |
| 2294 | cg02506421 | TTCTGTTGCAGGGACTAGGCCCTGGCCAGGCCCCCGAGGTGGCCCCCACG | | | | Rat | Human | |
| 2295 | cg25539759 | CGCATCATGGAAAAGCAGTTGCTCGAAGAACTCGAGCGACAGAGACAAGC | | | | Rat | Human | |
| 2296 | cg00994804 | CCTAACTCAATCGGCTTGTTGTGATGCGTATCCCCGTAGATGCCAGCACG | | | | Rat | Human | |
| 2297 | cg08438560 | CGCACAAACCTGACCAGCAGCGCAGAGGAGCCGTGGAAAAACCAACTATC | | | | Rat | Human | |
| 2298 | cg12203266 | CGAGGTGTAATCATTCTGGTTGAGAGAGAAAAAGATTAAGAGGGTGCAAC | | | | Rat | Human | |
| 2299 | cg18289982 | CGTGAGAATAATGAATGTGGAAATACAACTAAAAACATGGCACTCAAAAC | | | | Rat | Human | |
| 2300 | cg08000413 | GACAGCTTCAACTTCTTCACCAGAGAATCTCTTGCGGCTATTGAAAGACG | | | | Rat | Human | |
| 2301 | cg13219248 | CTAGCCAGCAGCAGAGACAGATGGCAGAGTTATTAGATGTGCATCATACG | | | | Rat | Human | |
| 2302 | cg04586876 | CGTGTCCAGAGGTCTCTTGTGGAACATTTCTACAGCTGTCTCCTTCAACT | | | | Rat | Human | |
| 2303 | cg00046033 | TTATCATTCGGAAGCTTTAATTCTATTGAAATGGCAGCTGCTCAGCTCCG | | | | Rat | Human | |
| 2304 | cg26048456 | TTCCCACTGTCTTCATCTGTGGCACTAATAGTTGTTAAGTATAACCCACG | | | | Rat | Human | |
| 2305 | cg24117712 | CGGAGGAAACGAGGAAAACTTGGGAATTTCCCGTGCTCCTGAAATATCTC | | | | Rat | Human | |
| 2306 | cg23705797 | CCATAAGCCGTCACTTATTCATCTTAATTTCAACAAAATTAAAGTAGTCG | | | | Rat | Human | |
| 2307 | cg02507211 | CGAACTCCCAGTTTTTGTTAGGGGCATAATCCCATTCTACTTCTTCAGCG | | | | Rat | Human | |
| 2308 | cg01066008 | CCTCCGGGAGGTTTCGTTTTTCCATTGAAGTCCCCCTGATTTACTGTACG | | | | Rat | Human | |
| 2309 | cg03533765 | CGCTGCAGGATGATAGTGACTTCTGAAATTATCACAATTATCATTCCATT | | | | Rat | Human | |
| 2310 | cg14650619 | CGGAGCTTTGGAGAGATGAAGTTTAAACAGTGTCCTACAGAGAACATGGC | | | | Rat | Human | |
| 2311 | cg01756262 | GCAAATTAATACATATATCAATTTTGTCAGAAACATGCTTAGTTCAATCG | | | | Rat | Human | |
| 2312 | cg01885247 | ATGGTTACCTGTTCAGCACCGATTCAAACTGCCGTTCTTTATTGACAGCG | | | | Rat | Human | |
| 2313 | cg02979795 | CGCCCGGGCTGCCTGTTCCAATATTAGATATAAACAAGTAAACTGTGTAA | | | | Rat | Human | |
| 2314 | cg17314023 | CGGACAGAGTCCAGATGGCTGTTCTACCGTTATGTTTCCCAAGATAATGG | | | | Rat | Human | |
| 2315 | cg20118823 | CGCACCTGCTGCTCCAGCCCCAGGATGACCTGCACGGCCTGCTGCAGGAT | | | | Rat | Human | |
| 2316 | cg18522824 | CGTGCTGTCGAGAGAAAGGTGGATCCCAACAACAGGAAACTACCTAAATC | | | | Rat | Human | |
| 2317 | cg18897683 | CGCATTGCGCAGGGTGCACCACCGCTTGGTCTCCCTGCAGCTTTAACAGC | | | | Rat | Human | |
| 2318 | cg18937321 | CGCCCAGGTGGCTTTCTTAAAGGGAAAGTTGCATCAGCCTCCCGAGGCTC | | | | Rat | Human | |
| 2319 | cg12743193 | AGGAGCTGTGGGTGCTGATCTGGCTGTACTCGCAGTCTTTCATGGGCCCG | | | | Rat | Human | |
| 2320 | cg24966735 | CGGCCCACCCTACATTTAGCGCATCATGTGATCCGGGTAAGTCATTTGCA | | | | Rat | Human | |
| 2321 | cg12934146 | TTGGGTACCCAAGCTGGCTTTTGTACTCTTCGGAGCTTCCTTGTTCAGCG | | | | Rat | Human | |
| 2322 | cg19839218 | CGTGAGCCCAGATCCGGACGAGCAGTTCACATTCCATTACGAAATTCTAC | | | | Rat | Human | |
| 2323 | cg19895248 | CGACTCAGCAAGGTCACTGTTCAGACTTGACTCGTGATTGGCTGCTGCGC | | | | Rat | Human | |
| 2324 | cg22262485 | CGAGTGATTTATGACTTCAGTCTTGGTTCACCAAGAGTTCACACGGCTTT | | | | Rat | Human | |
| 2325 | cg26450215 | GGAAATCATGCCCATGAATATAGTTTCCATCATTTCACCCTTGATAGACG | | | | Rat | Human | |
| 2326 | cg21375168 | CGAGATGCAATAATATTTGTGAACATGTTAATATAAAAAATGAATTTAAT | | | | Rat | Human | |
| 2327 | cg22040516 | CGGGATGCTCGGACTGCACAGAGCCACCATCAGGCAGCAGGTGACCAGGA | | | | Rat | Human | |
| 2328 | cg18253120 | TCAACTAAGAAAATACAGAGTGAACCATATGGCCTGGGTGGCTCGAGTCG | | | | Rat | Human | |
| 2329 | cg25565338 | TTCACAACCCCCTGAAAGCGTAAGTAAGACTAAAAAATGTACATATTCCG | | | | Rat | Human | |
| 2330 | cg22616024 | TTAAAAGAATCTTTATATTTCATTAAGCAAGCAGATCGGCGGATCTCCCG | | | | Rat | Human | |
| 2331 | cg22608267 | TGGAAGAGATTCTGCATTACTACACCTCAGTGCCTGCCTGGTTCCCTTCG | | | | Rat | Human | |
| 2332 | cg00056578 | GTTCGCACTGCTGTAAGGCTTTTTGCAAACTCAGAGGCTGCTGCTCCTCG | | | | Rat | Human | |
| 2333 | cg00075753 | CGCGGCAGACCACCCACGCCGAGGGCCATGGAACTGCTTAATAGAAACAG | | | | Rat | Human | |
| 2334 | cg00095474 | AATTAAAAATTCAAACCTTGTGTATATTTGAAAGCATGAATCTTAGCTCG | | | | Rat | Human | |
| 2335 | cg00116653 | CGATTTAGGTTTTAATTCCGGGGCTTTTGGAGCCTGTCAGCCCTGATGTA | | | | Rat | Human | |
| 2336 | cg00138195 | ATTAGGCCGCAAATCGCGCTGAGCCTCCCTAAGTGACAGCGAGAGAATCG | | | | Rat | Human | |
| 2337 | cg00166579 | CGGGCAGCGGCCGCAGCAGCTCCTTGATTCAATAGATGAGATGGAAGAAG | | | | Rat | Human | |
| 2338 | cg00170518 | TTACTTGCTTTAACTTCATACGGTCCACAGTGAAAGAGTTCATTCAGTCG | | | | Rat | Human | |
| 2339 | cg00187380 | ATGACCAAACTGCACATGAGCCACGGTAAACTTTAGGACTTCATTTTGCG | | | | Rat | Human | |
| 2340 | cg00236139 | CGGGTGTCTGACAGCCGGACAGCGGGATCTGCACAGCAAGTGAAATTCCC | | | | Rat | Human | |
| 2341 | cg00281640 | CGTGCTCATACGAGCAAAGGGCCCGGGCCATATAGTCCCAGTGTCCAGTT | | | | Rat | Human | |
| 2342 | cg00323965 | CGCGCCACCTATCTTTGTGGGGAGGGATAATTGAAGCGCCCTGAGCGTGA | | | | Rat | Human | |
| 2343 | cg00379872 | CGCACCAATAAAAATAATTGTGCAAGCCTAAATTTGGAAGTGTTCCCAGC | | | | Rat | Human | |
| 2344 | cg00386839 | CACAAGCAAAGAAGCATGTCCCATTTAAATACACCCACGCAGCCCCAGCG | | | | Rat | Human | |
| 2345 | cg00411555 | ATTTTTTTTTAAGCTCTGAGCTGTCAACGAGACGTTTCCTACCGATCTCG | | | | Rat | Human | |
| 2346 | cg00427623 | TTCTTTTCTTTCAACACAAAAGTTGGGTTGTGAATTTTCCGAGGTCGCCG | | | | Rat | Human | |
| 2347 | cg00487538 | TTTCTGAGGTTTAGTGAGTTCGATTAGCCGCGTGCTCAGAATCAAGTTCG | | | | Rat | Human | |
| 2348 | cg00490620 | CGCGGCGGGCTGAGTTTTGAAAACTTAGGATTCTAAATGTATAAATTGTT | | | | Rat | Human | |
| 2349 | cg00531009 | CTCCCATGGACCCTTCTAACTTTGTCACCATGATGGGCATGGGGGTGGCG | | | | Rat | Human | |
| 2350 | cg00553911 | AGGGCTGCTGCAGGTGCGTCTGCACGTGGGACTGGCAGCAGGGCGCCTCG | | | | Rat | Human | |
| 2351 | cg00571722 | ATCCCTCTGAAGGCCTTTAACTCCACCTGTGACTAACCCATTCATAATCG | | | | Rat | Human | |
| 2352 | cg00578263 | CACGATGATGATAATTGTAATTAGAAAGTGCCTGGCAATTTCTTATCACG | | | | Rat | Human | |
| 2353 | cg00591406 | GAAATTGTCCTTTGATTAAAAAAGATTCATCACCATTTCCAGCTCTGTCG | | | | Rat | Human | |
| 2354 | cg00716372 | CGCAGGACTTCCATGTCTGAGGGGATCAAGCAGCTCTTGAAAGATATGTT | | | | Rat | Human | |
| 2355 | cg00857360 | ATCTGTAGTTCAATTGTAAATTAGAATACACATCCCCCCAGGGACACACG | | | | Rat | Human | |
| 2356 | cg00902440 | CTTTTCAATGTATAAAATGGAATATTCTTACCTCAATTCCTCTGCCTACG | | | | Rat | Human | |
| 2357 | cg00959147 | CGCGCGGCCGAGCCCCACGCAGCCGCACAGACGTAGTCCACAACCATTTC | | | | Rat | Human | |
| 2358 | cg00967479 | GTGGGCAATTAGGCAGAACTTTGTTTGCACTGCCTCGGAAAATGAAACCG | | | | Rat | Human | |
| 2359 | cg00971828 | CGGCTTACTGAACATTCCAAATGCTGGTACCATGTAATTGGACATAATAG | | | | Rat | Human | |
| 2360 | cg00975228 | ACTTTGGCTGCTCAGTTGCATTTCACAGGAACCTAGATATCGAATCCTCG | | | | Rat | Human | |
| 2361 | cg00993893 | CGGAGGGAAGAAAAACTAAATTCACTTGTGAGGCGATTTTATGGCAGAGC | | | | Rat | Human | |
| 2362 | cg01016328 | TCAGCATCATACACCTGACCTGGGGCCACAGCTGGATAAAGAAAGTCACG | | | | Rat | Human | |
| 2363 | cg01096459 | TTCCTTTTAAGATTCTCCAGAAGGCTGCAAGGCCAGAACCACAAATATCG | | | | Rat | Human | |
| 2364 | cg01097631 | TTAGTTACTTTCCCACTTTGAGGTTAAACAAACCAGTGAATGGGACTCCG | | | | Rat | Human | |
| 2365 | cg01127764 | CGATCGGAATGCCAATAAAATCGATTAAAGGTTTAGTGTTTTACAAACAA | | | | Rat | Human | |
| 2366 | cg01267365 | CGGCATGAACGCGGTGCGGACGTGGATGCAGGGCGCCGGGGTGCTGGACG | | | | Rat | Human | |
| 2367 | cg01275157 | ATTTAGAGTCATTTAATCTACTCAATTTGCACGTTAATTAAACAGCATCG | | | | Rat | Human | |
| 2368 | cg01319626 | TGAGGTAACTATAAAACCCCGATTTGTTTACATTCCCTCCCCCAGAACCG | | | | Rat | Human | |
| 2369 | cg01324452 | GGAAGGCGATGGCCATGTTCATCTCCAGCATCTGCAGCAGGTCCACTGCG | | | | Rat | Human | |
| 2370 | cg01340046 | AAACCGTTTTCCACTCATTACAACAACAGTGCTGGATTTATTCTAATGCG | | | | Rat | Human | |
| 2371 | cg01343642 | ATAAAGCCACCATAAAATGTGAGACTTCTCCTCCTTCCTCACCCAGGACG | | | | Rat | Human | |
| 2372 | cg01343704 | AGGCCAAGGGCATAAAGCCCAATCTCGAAGGCGCAAAGGTGCAGGTTGCG | | | | Rat | Human | |
| 2373 | cg01476234 | CGCCATCTTACTGACTGGTCTGGATATATGTTCCTGGAAGAATGAGCCCT | | | | Rat | Human | |
| 2374 | cg01526505 | CGGCTGACAGATGACCATATGACACCCTTCAGACAGTACAGTATGTCACT | | | | Rat | Human | |
| 2375 | cg01533729 | ATGAAACTATTAAACCCGGGTGGAGGTCAGGCTGGAGGCGCCCCAGAGCG | | | | Rat | Human | |
| 2376 | cg01538162 | ACCCTTCTAACTTTGTCACCATGATGGGCATGGGGGTGGCGGGCATGCCG | | | | Rat | Human | |
| 2377 | cg01579422 | CGGATGAAGAAACCCAGGGCCATGCTCCTATTAATACAAGAATTTGTATT | | | | Rat | Human | |
| 2378 | cg01580126 | CATCTACCACGTCGGGGACTGGCTACAAGAGCGATGTACAAGCCAAATCG | | | | Rat | Human | |
| 2379 | cg01621100 | CGCCCAGTACAGCTCGGCCAGCACCCCCCAGTACCCGACAGCACACCCCC | | | | Rat | Human | |
| 2380 | cg01640606 | ATCACGTTTCGTTATCTTTAGTGGGAATCTGCAACGTGACACCGGATCCG | | | | Rat | Human | |
| 2381 | cg01768969 | TGGCCGCCAGCCCGTAGGTTTCCTGAGTTTGAAGTTGGGCGTGCCTGCCG | | | | Rat | Human | |
| 2382 | cg01783053 | TCAGTTAACTGGTGGTAGTAGCGTTCTATTAGATGCTTTGCAGCTGCTCG | | | | Rat | Human | |
| 2383 | cg01798687 | CGGGCGCAAGTCGGGGAACAAGCCTCCGTCCAAAACATGTCTGAAGGAGG | | | | Rat | Human | |
| 2384 | cg01802683 | CGGTGCCAAGATTGCTTTAAAAAATTCCCTTGGCCCCAGTTCAAACAGGA | | | | Rat | Human | |
| 2385 | cg01855583 | CGCAGAATACACTTTGGGAAACGCTCTAAGGAAACAGAAAATAAAATGTA | | | | Rat | Human | |
| 2386 | cg01918956 | TTTTCATGTATCCTTGATAACTGTTTCATAATGAGCTATGCGTCTTGACG | | | | Rat | Human | |
| 2387 | cg01939401 | CGCTTATGGTGGCCTGGGCTCGAATCCATGTTATGGAGGGGTTTTGCCTC | | | | Rat | Human | |
| 2388 | cg02044556 | TGATGTTGCCTGCGTCCAGTGCGGCAGCCCTCTTGTCAAAGCTTATGACG | | | | Rat | Human | |
| 2389 | cg02074136 | CGGGGTGCTGAGTGCGCAATTGCCTCAAAGAAAAGTAGACAGGAGACAAG | | | | Rat | Human | |
| 2390 | cg02132378 | CGTATTTCATGTCATTATGTTGGATAATATAACAAATGATGCAATCAGAG | | | | Rat | Human | |
| 2391 | cg02157959 | AGGGCATCAGTAATCTTTGCTCAAGGGCACGGCTAACAGACAGATAGACG | | | | Rat | Human | |
| 2392 | cg02179615 | CGGGCCAGGTGGGCCTCTGACACACAGCAGGAATTTTCCACTGGCGCATC | | | | Rat | Human | |
| 2393 | cg02241397 | CGGCCATTATGGGCCAAATGATTCATTTTAATTTGGCAATTTCACCGGAG | | | | Rat | Human | |
| 2394 | cg02285385 | CGCTGGTGACTCGGGGAGCCGCAAACTTCAATTTAAATTTACAGTGCTGC | | | | Rat | Human | |
| 2395 | cg02495519 | TCACAGCTGCTCCAGAGGTTCCTCCAGTCAAACCCTTTCTGGAAACGACG | | | | Rat | Human | |
| 2396 | cg02515765 | CAAGGAGAATATGACAGCCACCTCCCTTGGCCCTTCCAGGGTACAATACG | | | | Rat | Human | |
| 2397 | cg02596500 | TTCTGAATGCTATTTAAACATTGGCAGGGGGCCTCATTAAAGATGAATCG | | | | Rat | Human | |
| 2398 | cg02611007 | CGGTAATTACAAGACAGAAAATTAACTAGAACTCTTATTAAGGCTTTGTT | | | | Rat | Human | |
| 2399 | cg02658132 | AAATGGAACTTGTGTCAGCAAAGAACTGATTTTCATACAGACTTCTTTCG | | | | Rat | Human | |
| 2400 | cg02673411 | CGGCTCGATGCTGGACATACTTGACCGGGGTCATCCTAGGGAAAACACTA | | | | Rat | Human | |
| 2401 | cg02711775 | CTGGAGGTGATGGATGTTTTCCTCCGGTTCTCAGGGCTCCACCTCTTTCG | | | | Rat | Human | |
| 2402 | cg02722823 | CGCTGCAGTCGTAAGGTTTCTCTGCAGTGTGAATTCTCTGGTGCACAATA | | | | Rat | Human | |
| 2403 | cg02724885 | GTCCTCCTGGAGGGGATCTCTGGCCAAGGAGTGTACGGTGAGTGGAATCG | | | | Rat | Human | |
| 2404 | cg02739710 | CGGGAGCCCTGACTAGAAAGAGGGTTCTGGAAATGTGGTGTGTTTCTCAT | | | | Rat | Human | |
| 2405 | cg02779868 | CGCACCATGGCAACATCAACCCGTCAAAGCAAATGAAGGGTAGTAAAAGT | | | | Rat | Human | |
| 2406 | cg02810085 | TTTTTATCCAAATGGCTCAATTACCGCAAAGCCCCACAGAGAAAATTACG | | | | Rat | Human | |
| 2407 | cg02841886 | CGGTAGTCGTGGCGACCAAGCAACCCGGCAACGCGAGTCAACAACAACAA | | | | Rat | Human | |
| 2408 | cg02882406 | TTCACGATCAGTATATTCTGAGGGTTTCTCCGGATTTGGAATGGCATGCG | | | | Rat | Human | |
| 2409 | cg02897704 | CCTCTTGAACATTTACCGTAACCCTCAAAACTCTTCCCAGTCTGCTGACG | | | | Rat | Human | |
| 2410 | cg02944016 | CGGTCGACCGCTGTACTAAAAGGGTTCTCTTGAGAATCCACCAGAAGTCT | | | | Rat | Human | |
| 2411 | cg03036064 | GGAAGGGCTCGCCGCCGTTGGGCTGCTGGTGGTTCTGCAGGGTCTGCTCG | | | | Rat | Human | |
| 2412 | cg03057744 | CGCAGTAAAAACTAACCCTTGGGTTGAATCTTTACTTACAGAGGTATTTT | | | | Rat | Human | |
| 2413 | cg03076661 | CGCATCACCACCTATTTTGACATGCACGGGGCCGTTTGTTGTCTGTCGAA | | | | Rat | Human | |
| 2414 | cg03220194 | TATTTTCCAAAAGCTCGGTCTGAGGCCCCTCAGTCTTGCTTCCTAACCCG | | | | Rat | Human | |
| 2415 | cg03255984 | CGCTGCACCATGAAGGGGAACCTCAAGTCGCACATCCGTATCAAGCACAG | | | | Rat | Human | |
| 2416 | cg03257592 | CGCTGGTAAATCTTTATTAATTTTAATTGGCGGCACCCATTTATAAGAAC | | | | Rat | Human | |
| 2417 | cg03265102 | TGTTCTGTCTTACTCTGCGCCTAGCATTGGAAATGAAAGTGACATTTACG | | | | Rat | Human | |
| 2418 | cg03267882 | TGACACTGTCAACACTGCCCTGGTGGAAGACTCCGAGGGGCTGGACTGCG | | | | Rat | Human | |
| 2419 | cg03342744 | CGGAGTGTTTATGTCAACTACATATTCCCCTAGATACGAATTTGTGACCA | | | | Rat | Human | |
| 2420 | cg03375386 | CGGGCTCTGTATTTCTGGAACAATGAGTATATCCTAAGCCTCATTGAGGA | | | | Rat | Human | |
| 2421 | cg03382217 | TAAAAATTCCCTTCATGAGCATCGACGGGTACAAATTATTTTGTCAATCG | | | | Rat | Human | |
| 2422 | cg03383661 | CGGCAGCTGCACATGTGGGTTTGTTTATAAGAACAAGGTTAGAAACTCGC | | | | Rat | Human | |
| 2423 | cg03532626 | TTTGTATTCTGCATGGTACGTACAATAGCAGACACCATCTGAGGAGAACG | | | | Rat | Human | |
| 2424 | cg03542972 | CCTTCATGTTCATGGTGCTGGAGGTGGTGGTGAGCCGGGTGACCTCGTCG | | | | Rat | Human | |
| 2425 | cg03659513 | CGTTGGCACTTCCACTGTGTACTTTTACTATAAATTGTTGTAGCATTTAT | | | | Rat | Human | |
| 2426 | cg03697136 | CGGAGCTGACGCTTTATCAGGCAGTCGGAAAAACTTTGACCAATCATTTT | | | | Rat | Human | |
| 2427 | cg03734211 | CGGGATATTGCACCACGCTCGCCTCTTGCTGAGCCCCATAAAGGGACTGT | | | | Rat | Human | |
| 2428 | cg03793834 | CGTTTCTAATAATAGAGCAGATGAAGTAATATTGCAATTGATGTTCCCGG | | | | Rat | Human | |
| 2429 | cg03888920 | GCCATTGCTGCTGCAAGCCTTATAACTGCCTTCAGTGCCTGGACAAGACG | | | | Rat | Human | |
| 2430 | cg03932150 | CGTGGCTAAATTGTTAATCAGGGTAATTTAATATAGTTTTCAATATGCCT | | | | Rat | Human | |
| 2431 | cg04138506 | CGGGATTCCAGCATCACCACCAGGAGCTGTACCCACCACCGCCTCCGCGC | | | | Rat | Human | |
| 2432 | cg04182635 | CGGCATGCAAAGGGTGAGTTTTATTTCCATAGATTTTTAAAATTGCTGTT | | | | Rat | Human | |
| 2433 | cg04287515 | GATTGTGTCTGCCTCTACACATATCTGTAATTGATTGCCTAAACCCTACG | | | | Rat | Human | |
| 2434 | cg04326641 | TGGACCGCGGATGGTACGTTCCGCACGTGAGCTGGGTGCTGGTCTGGCCG | | | | Rat | Human | |
| 2435 | cg04441307 | CCTGATTGGCTTCCATCCATCGTGTAGCGTCCTGCAAGTGACTAAACTCG | | | | Rat | Human | |
| 2436 | cg04580151 | CGGACCAAGGGGAAATGAAGAAGCCCCAATAAATCACCATGAAGCCACCA | | | | Rat | Human | |
| 2437 | cg04609744 | GTTAATATATTAGCACACAGCATTGATCAAATGGCTAATCCAATCACACG | | | | Rat | Human | |
| 2438 | cg04638909 | CCAGAAATTAATGGCTATGAGTTCTTTTTTGATCAACTCAAACTATGTCG | | | | Rat | Human | |
| 2439 | cg04664817 | CGCCCAAAGGGGATACTGCATCCTTAGGAAGAAATATGTCCTCATTTTTC | | | | Rat | Human | |
| 2440 | cg04701542 | CGCCTGGGAAAGCCATCTTGAACTGAAAACGGCAAAGATAGAGAGAGGCC | | | | Rat | Human | |
| 2441 | cg04830825 | GGCAATTTCTGGAACTCTTCCCACGGGAAGTTTTCCGGGGCTGCCTTCCG | | | | Rat | Human | |
| 2442 | cg04842607 | GCTGGGAATTCAGGTTAGTGGAGGCCGAGGATCAAAGCGCTCACCTCACG | | | | Rat | Human | |
| 2443 | cg04921727 | TACCGTCCGGCGTTGCCTGCTGGTGGGGTTTTTTACATTTGACGTAATCG | | | | Rat | Human | |
| 2444 | cg04949439 | CCCTCAATAAATGCTTTTTGTAATCAACTTAAAGTATAATACCTACCACG | | | | Rat | Human | |
| 2445 | cg05015154 | ATTAGTCGTCTCACCCAATAATTAGTTTTTTGTTTCCCTGTCTGTTTTCG | | | | Rat | Human | |
| 2446 | cg05019982 | CGCCTTGTAAAAGCAGATTACAGATGAAATGGGGCACATATAAATGCTAA | | | | Rat | Human | |
| 2447 | cg05047982 | CGGCCCAGGTTCGCCAGCAGCCCAAGAGCCTCAAAGTACGTGCAGGCCAG | | | | Rat | Human | |
| 2448 | cg05082095 | ATCTCCAGAACAAAGACAACATTGACAAACCCAACAAAAACAAACAGCCG | | | | Rat | Human | |
| 2449 | cg05098471 | CACAATGCATTGGGCTGCAGCAAGTAGGCTCCTCGGCAGGGGTGGGTGCG | | | | Rat | Human | |
| 2450 | cg05098623 | TCAGTTCTGTCGAATAGGGCATCATCACCTCCCTCCAGTCTGCGTCATCG | | | | Rat | Human | |
| 2451 | cg05147550 | AATTACCCATCAAGCCATATCTTTTCAGCCCAGAAATGCGCAATAACACG | | | | Rat | Human | |
| 2452 | cg05202136 | CGTCATTTTGATGTCACGTTTCCATAGCAACAGAGCAACTGCATAAGCTA | | | | Rat | Human | |
| 2453 | cg05232001 | TCTCAGTCCTTAATTCCAGTCAATTCCAGTCGAAAGGCTGATAGGCATCG | | | | Rat | Human | |
| 2454 | cg05234151 | CGTGTGGCTATAAACCCTTCCGTTGTGAGGTTTGTAACTACTCTACCACT | | | | Rat | Human | |
| 2455 | cg05273821 | GGTGGGTACAGCTCCTGGTGGTGATGCTGGAATCCCGATTCCCTGGTCCG | | | | Rat | Human | |
| 2456 | cg05337739 | TGGTCCTCAGTGACTTCATGAGCCCCCTGTTTACCTTACATGGTCACACG | | | | Rat | Human | |
| 2457 | cg05354872 | GGGGAATATTAATGATTCAATTTTCAACCGGGTTCCACTCCAGGGCCTCG | | | | Rat | Human | |
| 2458 | cg05396010 | CGGCATTTCTTAGGCGAGCCAGGGTCTGCTCACAGCCATGCCAGAGGGGT | | | | Rat | Human | |
| 2459 | cg05396320 | CTATGATGATTAGTGTAACAAAATAGATCCAGGGCCAGTCCCTTCCTACG | | | | Rat | Human | |
| 2460 | cg05397631 | CGCAGATCAGAATACAAAAGGAGAAGCAAGAAAATGACACCCAGCCAGGA | | | | Rat | Human | |
| 2461 | cg05543909 | AAGAATGTAAAATCACCGGCATAGATAGGTATATGGGAAAACAACCCACG | | | | Rat | Human | |
| 2462 | cg05578172 | GGGTAAACCGACCTGGTCCGGCATGCATTCATCATGTAATTTTCCAGACG | | | | Rat | Human | |
| 2463 | cg05679822 | GTTGCTGGGTGACTCAGTGAGCTTTGCATCCGGTCTCATTCATAAATCCG | | | | Rat | Human | |
| 2464 | cg05686715 | CGTCTTTCTTACTAATAGGATTCTGTCTGCTTCAGCAGGCAGTAATTGAA | | | | Rat | Human | |
| 2465 | cg05698015 | CGGCAGCAGCGAGAGCGGAAGACATTAGAGACCCGAAAAATAAGGGAAAA | | | | Rat | Human | |
| 2466 | cg05789487 | CGTGACCCTGACAGGTCCTGCCTATGGCGCGGCAGACCACCCACGCCGAG | | | | Rat | Human | |
| 2467 | cg05791038 | CGCTTTCCCACACGCCGCTGCGCCCCTTCAGCCCGCCCAGCCACTCGCCG | | | | Rat | Human | |
| 2468 | cg05791494 | CGAACAGACCGCTTTTATTGGCTTTTAGTGCTATTACTGCTGTCATAAGT | | | | Rat | Human | |
| 2469 | cg05798330 | TTTCCATGATGAATTGCCCGAAGCCCCTGAATCACAAACCATTTTTTCCG | | | | Rat | Human | |
| 2470 | cg05820448 | CTTGCTACCATTTAAAATCAGACTCTTTTTGTCTTTTGATTGCTGTCTCG | | | | Rat | Human | |
| 2471 | cg05852043 | CGGGTTTTGTGTTTTTCTAAAATAAAGAGAAGGATTATCAGAGAGGAGAG | | | | Rat | Human | |
| 2472 | cg05895882 | ATCTTGACCTGCTGGTCACTAAGATTCAAGCGGTCTGAGAGTTCCCTCCG | | | | Rat | Human | |
| 2473 | cg05958112 | TGAGCTTTTCCCCTGGAGGATCCTCATGCCCCATCTCCACCTAGGGATCG | | | | Rat | Human | |
| 2474 | cg05983884 | CGGCTCACTTCATGGACATTAATTTTAATGGGACGATTATTACCCTGTTT | | | | Rat | Human | |
| 2475 | cg06014896 | CGCTCAAGTGCACTTTCCGGTCCCTCCAGGTCAGCAGGCTGCCCAGCAGC | | | | Rat | Human | |
| 2476 | cg06024489 | CGCTATCCCGGCTTTAGGGGGCATCTCATTACCTCCAGGGGTCATCGTCA | | | | Rat | Human | |
| 2477 | cg06070696 | CGGAAGTGTGTGTTGACACAAATGATTAAGGCCAGGATTCCTACAATTGA | | | | Rat | Human | |
| 2478 | cg06097717 | AAGCTCTGTCCCATTTGGCTAATTCAGAAGTTAATGGAAGGTCTCTTTCG | | | | Rat | Human | |
| 2479 | cg06295366 | GGTGGGCTTTGTCACCAGCACGCTGGGTCTGGCATGCCCAGCTATGCTCG | | | | Rat | Human | |
| 2480 | cg06425973 | CGGAAATTTGAATACCACAGTAGCATGGAGTGTGACCTCATGGAGACTGA | | | | Rat | Human | |
| 2481 | cg06681652 | GTGCTGTCCCTCTCCACTTTCCACATCCACAAGCGTAGGATGAAGAAGCG | | | | Rat | Human | |
| 2482 | cg06683527 | TTCTTTTCATTATTTCCCTCCAAAGGTCAGTTATCCTCTCAGGCGGGGCG | | | | Rat | Human | |
| 2483 | cg06685968 | CGGTTCTCATCCACATGACCGACAGCGGCCAATGGAAGGGCCGAACAACT | | | | Rat | Human | |
| 2484 | cg06695854 | GGTTCTTAAACCAGTTGCTGACCTGGGTGGTGGTGAGGCCGGTGGCCTCG | | | | Rat | Human | |
| 2485 | cg06710083 | CGACAGGTCAGACTTGAACAGGCAAGGTCCACCACCAACCATCGTCGAGT | | | | Rat | Human | |
| 2486 | cg06829904 | GTCATTTTATGGTCAATTTTCAGAGTGTAACTGCACTTTAAGCACTAACG | | | | Rat | Human | |
| 2487 | cg06834901 | CGCAAGTAGGAAGCTTTTTGCACTCCACCACCTCTGGCCGCTGGGAAGAC | | | | Rat | Human | |
| 2488 | cg06878994 | CGCCAGGGCTGCCAGCAATTTGCTTATCAGAGTCACTTTGAAACAGCTTT | | | | Rat | Human | |
| 2489 | cg06925219 | CTTCTGCACTCCGTGTCCTAAATATGGATAATTAGCATCAGTTTATCACG | | | | Rat | Human | |
| 2490 | cg06973724 | ATCTGTGATTGGTTATCTCTCTATCATGCATTGCTTCACAAGGGGAAACG | | | | Rat | Human | |
| 2491 | cg06998842 | ATAAGCCGTCTGCAGTATAGTTAACTTTCAATTTCAGCCAATTTTCGACG | | | | Rat | Human | |
| 2492 | cg07043724 | ATAACTTGTATTAAAATACCAATTACATTTATAGGACCTTTTAAAACTCG | | | | Rat | Human | |
| 2493 | cg07094276 | TATGTTAGAAACATACAGTGTGGCATTTAGTATATACACTCCCTTGCTCG | | | | Rat | Human | |
| 2494 | cg07228206 | CACCACCTTCTCCTTGGTAACAGATAAGAGTCTTTCCACTTCAATCTTCG | | | | Rat | Human | |
| 2495 | cg07305197 | GACAGAGCAATAAATCAACTAATAACTGGAACCTCATTTTCTGCTCCACG | | | | Rat | Human | |
| 2496 | cg07335109 | CGGCTGTATTTCGGGATCTATTATAGTCTGTCCAGACAGATCCCATTGTA | | | | Rat | Human | |
| 2497 | cg07341770 | CGCCTCCAGAATAACGAGCCGAAAACATGTGTACTGTAAATAAAGCCTGT | | | | Rat | Human | |
| 2498 | cg07350767 | ACTGATGTGCAGCAGAAAATGGCTCCTTTCCCCTTTCCCTCCACCGAACG | | | | Rat | Human | |
| 2499 | cg07392671 | CGCCGCCGCCGCCAAGGGGAGTTTCCAGGAAGTGGCCATATTGGATCCAT | | | | Rat | Human | |
| 2500 | cg07525158 | GTCGGTCATGGCGCCCTGGTCCCTCACGTACTGGTTAAAATTAGGAGACG | | | | Rat | Human | |
| 2501 | cg07540568 | AGTTGGTGTGGTACAGCTTGGCAGCCTCCTGAGCCGGGAGCATCGCGGCG | | | | Rat | Human | |
| 2502 | cg07558872 | CGGCGGAATTTTTAAAGAGGGATGTGGTTACCTTGAGCAGAAATACCCAA | | | | Rat | Human | |
| 2503 | cg07607355 | CGGAGCTATTTAACCTGAGCATCCCCAGGTGTACGGAGGCGCCTGGCTGT | | | | Rat | Human | |
| 2504 | cg07639660 | ATGACCTTTGATGTGTTTAAATAACATGGCAAGTCTGAGCACACACCTCG | | | | Rat | Human | |
| 2505 | cg07680301 | TTTTTTTTTTTCAAAGAAAGTCACTTTTAATTCCCAGGGCTCCCCGCTCG | | | | Rat | Human | |
| 2506 | cg07720974 | TGTGTAGCGTGGCCAGTAGCAGCGACATCAGCTTGGGTTCGACCAAAACG | | | | Rat | Human | |
| 2507 | cg07795407 | CGGGCTTTGATTGACAGCTGGAGTGGCAAAAAGCCATGAAACACGACAGT | | | | Rat | Human | |
| 2508 | cg07806359 | TAGGTGCTGGTCCTTGGCTGTTTGCAACCACTTTTCTGATGCGGTTGACG | | | | Rat | Human | |
| 2509 | cg07859114 | CCGCTGTGGTGAGAACAGAGTTGACACTTCTTTCCTTTCCACTTACTGCG | | | | Rat | Human | |
| 2510 | cg08003189 | CGGGCTAATTAGCCAATCAAATTAAATGCAGCACCTGCTCCCAGCCTGAT | | | | Rat | Human | |
| 2511 | cg08013737 | CGGTAACTTATAACTTATATATTAGGGCCCATGTGACCTAATAAAGAACA | | | | Rat | Human | |
| 2512 | cg08124155 | CGGGGTTTAGGGTTTTTGTAATCCCCCTAAACTGTCTTTGTTCGAAATCT | | | | Rat | Human | |
| 2513 | cg08129342 | CATTGACCCTGAAACCTATGAGGACCCAAATAGAGCTGTCCATCAATTCG | | | | Rat | Human | |
| 2514 | cg08153819 | CCAGTTTGCTGGACCAGTTCCCTCGGGCCTTATTCTCATCCCCTTGCTCG | | | | Rat | Human | |
| 2515 | cg08154911 | CGGCCAGGATGGTATTCAATTTGTCCTGATCACTGGGCTGCAGTGAGTTC | | | | Rat | Human | |
| 2516 | cg08215925 | CGCGGAGGGAGATGTTGTTATTTTTTGCTGTTGTGCGTCCCTGACGTCAC | | | | Rat | Human | |
| 2517 | cg08232037 | CAGTAATCCAATAAGAGCATTGATTAGGCTACAATGATTCAATTCAAGCG | | | | Rat | Human | |
| 2518 | cg08317235 | CGTCGGCCTACCCCAGCCACCCCATGCCCTACAGCTCCGTGTTGACTCAG | | | | Rat | Human | |
| 2519 | cg08352408 | TACTCCACATCAGTAAATGTTTACGTAACCTGTCCTTTCCCAAGAATCCG | | | | Rat | Human | |
| 2520 | cg08368788 | GCGGCCCTAGAGTTAATCCCATCAGCCGAGGTGAGGCACCTGTTACCCCG | | | | Rat | Human | |
| 2521 | cg08460572 | CGTGTTTAAAAACAGTGTTCTGACACTTTATTGCACAATGAGCTCTTACA | | | | Rat | Human | |
| 2522 | cg08492897 | AAAAAATCGCAAAAAATCCCTAAAAATCCGGGATGCTCCCCCTTTTCTCG | | | | Rat | Human | |
| 2523 | cg08515724 | TGTGGTCACCTGGCATTTGGGGCGGCGTGAGGCCCTGCTGGAAGGGGTCG | | | | Rat | Human | |
| 2524 | cg08759703 | CGTCTGTTCTGTGGACAACTGTTACTGTTCTTCCGTGGCCAACCATGGCG | | | | Rat | Human | |
| 2525 | cg08827873 | AAAAAGCATTAGTAACACTCCTGGTTTTAAATGAACCCTATTAATATTCG | | | | Rat | Human | |
| 2526 | cg08987848 | CGAAGAATATAGTGGGCTTTATTAGCACCAGTTTCGCTCCCTGACTGACG | | | | Rat | Human | |
| 2527 | cg09035699 | CGCTCAAACATGTGTGCTAAAGGCAAATAGGAAATGTGCACATCCGCACA | | | | Rat | Human | |
| 2528 | cg09075088 | TGGGCAGGTGCCATCCCGCGCCCTAAAAATAACGGCAGGGAACTAGGTCG | | | | Rat | Human | |
| 2529 | cg09130815 | CGCAAAGTGCCCAGTCCGGAGGCGTTTCTGGGCAAACCCTGGTCCTCCTG | | | | Rat | Human | |
| 2530 | cg09155589 | CGCCCCGTTCTTGACCAAACATGACAGACTCTGAACATGCAGGGCACGAC | | | | Rat | Human | |
| 2531 | cg09175523 | CGGCCTCGGCTCCTAATTGGAAGCATTGGGCATTATCAACGGAGGATTTG | | | | Rat | Human | |
| 2532 | cg09204130 | CGCTGGTCTCCGTGTTGGTGGGTTTATAATCTACATAAAATTCCTGGGTG | | | | Rat | Human | |
| 2533 | cg09260635 | CGCTGACACTCCAGTATTTAATTATGCAGCTGTCAGAGCGGCCCTTTCGA | | | | Rat | Human | |
| 2534 | cg09355828 | CGCACGCTGTCTCGCCATGGCTTTGATGTCTGTGCCAAGCTGGCTTGGGA | | | | Rat | Human | |
| 2535 | cg09530369 | GATGGAAAACTTTTAAGCCCTGGCTATTAGCACGCGGCCAACAAAAGCCG | | | | Rat | Human | |
| 2536 | cg09531328 | CGCCACTTTATTGAAAATAGCCTGTAAGTTGTTATTAAATGTATTGACTG | | | | Rat | Human | |
| 2537 | cg09598225 | ACCTGCTTGTCCTTCTGCAGCTGCTTCTCGATCTTTTTGTTGGCCTCACG | | | | Rat | Human | |
| 2538 | cg09610735 | GGGTCACGTGCTCCCCTCATTCCTTAAGGGCGGCCTGGGAATTAGTGTCG | | | | Rat | Human | |
| 2539 | cg09643584 | TCATAAGTGTCAGGACATACCAAGGTTAGCGGTCACTGGAAGTGTGCTCG | | | | Rat | Human | |
| 2540 | cg09667338 | GATTTATAAATATTGTGCACTATCTGCTGTGGCATGGCTCTCATGGGACG | | | | Rat | Human | |
| 2541 | cg09786757 | TCCCATCTGTGGTCTCCAAGTGTCTGCCTCTTGATTTAACTTTGTCCCCG | | | | Rat | Human | |
| 2542 | cg09789157 | GCCTACCACAAGTCGGTGTGGCGGGGGGTGGAGGCCAAGCTGCACCTGCG | | | | Rat | Human | |
| 2543 | cg09798507 | TTCAGTAGAAAATGACGGGTGATTTTTGCCCTTGTCTCCCGAGATCTCCG | | | | Rat | Human | |
| 2544 | cg09802094 | CCGAAGCGACCCTGACATTTAGAAAGACTCCAATTGCCTCGTTAAAATCG | | | | Rat | Human | |
| 2545 | cg09919752 | CGCCCCGAGGTCTTTCTGAGCAGACTGCTAAGCACTGGCTGTCTCCCCCG | | | | Rat | Human | |
| 2546 | cg09955247 | TTAATAATCTGTCATCAGGACCAGAGGCAGTCATAAATTATATGGGAGCG | | | | Rat | Human | |
| 2547 | cg10116181 | GAGTGATCCGGCAATGCGCTCCTGACCTTGCAGGACAGCTAATCCATCCG | | | | Rat | Human | |
| 2548 | cg10155596 | CGACGGCCTAAAGTTCTTCAAAGAGAGGTAGATGTGTATGATATTTTCAT | | | | Rat | Human | |
| 2549 | cg10210458 | TGGTGAAGGCATTTAACAAAGACTTCATTTTCCCTCTGACTTTGGCAGCG | | | | Rat | Human | |
| 2550 | cg10251050 | CCTGCTTAGGATGAGAACAAGTTGAGCAGTGCCAACGGCCATGAAGAACG | | | | Rat | Human | |
| 2551 | cg10319365 | CGCCTGTAGGAAATATTAATAGACTATTATGAGGTCCTACAACTAATTTT | | | | Rat | Human | |
| 2552 | cg10334858 | TTGACTTGACGTTTGGATCTAAGAGGCCTTCCTTCGCCATCCATTCATCG | | | | Rat | Human | |
| 2553 | cg10423457 | CGCCGTGATCAGTTGGACTAAGGATGGGGTGCACTTGGGGCCCAACAATA | | | | Rat | Human | |
| 2554 | cg10481534 | CGAGGGCTGGGGCCCCCTCAGTTTGTGTAAACCTGAGTTCCGATCACACG | | | | Rat | Human | |
| 2555 | cg10587989 | CGCCTCCTGAACTAAAGATAGAAACAGCTGGCCCTTCCAGGCAGCTAAAA | | | | Rat | Human | |
| 2556 | cg10588150 | CGCGCCTCTCCCCGGCCGGGGGGACACTTGGTTCATTCCAGCCGCAGAAG | | | | Rat | Human | |
| 2557 | cg10668528 | CGGATCCTGGGGTGGGGGAACTCCTCACTATAATCCAGCAACTTGCCAAC | | | | Rat | Human | |
| 2558 | cg10822690 | CGATTGCTGTTTGAATAACAGATTAATTTAATAATCAGTGGGGTATTGGA | | | | Rat | Human | |
| 2559 | cg10829846 | ACATTTAGTCCGGGGGTGACTCTTCTCCTTGGCACTTGTAGAATAAATCG | | | | Rat | Human | |
| 2560 | cg10901302 | CCTCTTTTGTTTATCTGACAGCTAAATATCAAGGCAATCACCGCCTCGCG | | | | Rat | Human | |
| 2561 | cg10901883 | TTGTGCGTTTCCCAAAGTCTACAGGCTTTTGATGAAAAATTCATGGCACG | | | | Rat | Human | |
| 2562 | cg10918079 | TCCATGTCGGCTCGGAACAGATTCGCCTCCATGTGTCTCTGCGTGGTACG | | | | Rat | Human | |
| 2563 | cg10939684 | CGGAAAATGAAAATATTTATTTTAAAAATTGAAATGTTTGGAACCTTTAG | | | | Rat | Human | |
| 2564 | cg10939790 | CGGGTTCTTGTCCGGGTGGTACTTGAGCGCCAGCTTCCGATAGGCCTTCT | | | | Rat | Human | |
| 2565 | cg10988592 | TGGAAGGATGCTGAAATGACAGGCCTAGTAGACGCTTGTCTTAATCATCG | | | | Rat | Human | |
| 2566 | cg11079986 | CGGGCACTACTCCATGCACTGTTTACACTCGGCGGGCCATTCGCAGCCCG | | | | Rat | Human | |
| 2567 | cg11094341 | ACCTCTTGTTCACCAGATCACTTTGTGTGCACTTGTATATTTCATTGTCG | | | | Rat | Human | |
| 2568 | cg11102919 | CGCCTAATTAAATTAATTAGGGATTCTTGTCAATCCTGGATTAATGGCCA | | | | Rat | Human | |
| 2569 | cg11103841 | TAACACACCTCGTGCACGACAAAAATTTGCTCCTGGCAATCATGGTTTCG | | | | Rat | Human | |
| 2570 | cg11202178 | AGCCCTCCCACCCCCTGAGATCAAGGCAATGGTGGTTTTACAGGCTGACG | | | | Rat | Human | |
| 2571 | cg11250627 | CGGTGTCTAAATGCATTCATATTTTTATGATTGTTTTGTAAATATCTTTG | | | | Rat | Human | |
| 2572 | cg11356333 | CGCTGACCCTGCTGTTTAGCCCATTAATGATGATGCACAAGCTTCACTTT | | | | Rat | Human | |
| 2573 | cg11382709 | GACGCAGCCAGCCATTCAGGAAGGCAGCGCTGGAGAGCGGGAGGCAGCCG | | | | Rat | Human | |
| 2574 | cg11392697 | TGGGCACTTGCACAGAGATGATCTCTGCCATCATTTCCGGAAAGTCCACG | | | | Rat | Human | |
| 2575 | cg11534937 | CTCCCCTCGGCGGGGCTGAATGATCAAAACTGGTGGTGAGAATTTTTCCG | | | | Rat | Human | |
| 2576 | cg11545945 | GTGATGACAAATGCCCGTTATCAGCGAACGAGGCCGATGACAAATGGGCG | | | | Rat | Human | |
| 2577 | cg11548411 | TATCCTCTAATATAAATGTCAGTGATGCTATTTTCCGCGCAGTCATTTCG | | | | Rat | Human | |
| 2578 | cg11577341 | CGCCCGATTATGCAGCCAATTAAGTTATTTGGAATGGTGAGTTCATGGGT | | | | Rat | Human | |
| 2579 | cg11617475 | GGACGTGTCTCTGCTGCACCCTCTTGAAGCGGCAGGACTGGGCATAGCCG | | | | Rat | Human | |
| 2580 | cg11670972 | GAACTGTTATTGTTTGTGATCCGCTCCATTTTATGGAAACAAGCTCTGCG | | | | Rat | Human | |
| 2581 | cg11758048 | ATGGCCTCCAACTTTAACGACATAGTCAAGCAGGGCTACGTGAAAATCCG | | | | Rat | Human | |
| 2582 | cg11789731 | CGGTAGAAGATCCAGCATCCAAAAGTGACCCAGTGACTGGCCCAGCTGAG | | | | Rat | Human | |
| 2583 | cg11900861 | TCATCAGAGTTCCCCCTTTTGTTCCAAGTCCCGGAGCAGACAAAGAGGCG | | | | Rat | Human | |
| 2584 | cg11994228 | CGCCTCCTCTGGGCCTGGGAGAGAACTGCCCCTACAGGGTGGCTTGCAAG | | | | Rat | Human | |
| 2585 | cg12012631 | CGGGCAGCAGAAGATTTCGTGACACTTAGCACGAAAGGGGGACTCTTTTT | | | | Rat | Human | |
| 2586 | cg12035862 | TTCCCTTGAGGCAGGTGATGTAGCGGTGACAGAAGTTGTCGCACAGGTCG | | | | Rat | Human | |
| 2587 | cg12063651 | CGCGCTCTTAATAGACCTGTTTCAACAATGTATCTTGAAAAGGAATACAT | | | | Rat | Human | |
| 2588 | cg12163477 | CGGGAAGGCCTGAGGGAAGCCCACCCGCCCCAGAGACTGCCCACCCTGGG | | | | Rat | Human | |
| 2589 | cg12172899 | CGGAACAGGATGACTTTGCCAATATCCCTGACCTGCAAAACCCAGGAACC | | | | Rat | Human | |
| 2590 | cg12191257 | CGGTGATGCAGAAGGTATGTGCTTTTTCAGTTCTGGTGCTGATGCTGTGT | | | | Rat | Human | |
| 2591 | cg12219231 | CGTTGGTCTGGCGGCCACTTTCTTTGAACATTAGCTCGCTTTCAGCTCCA | | | | Rat | Human | |
| 2592 | cg12234807 | CGCGGAAATATGAGCGAGTAATTTTATAAGTAACAAAGGATCAGGGATGG | | | | Rat | Human | |
| 2593 | cg12249796 | CATTACAGATTAAAATGAGCCACATTTGCAGTTGATGGTATCTGTTTTCG | | | | Rat | Human | |
| 2594 | cg12387371 | CGGCCTGCAAGAGAGGCCTGGAAGAGAAGCAACTTTAGTTGTGCAAGCAC | | | | Rat | Human | |
| 2595 | cg12396207 | CGGTAAGGCAGGCTGCATGGAAGCCTGAGACATATGAGACATCATCATTC | | | | Rat | Human | |
| 2596 | cg12556490 | CGCAGTGACTTGGGGCCAAATTGGAGTTTATATGTCAAAACAGGCTCAGA | | | | Rat | Human | |
| 2597 | cg12600656 | CGCTTCCTATGGATAACGTGCCGGGGCTGACCTTGCCATCTGGGGACGGA | | | | Rat | Human | |
| 2598 | cg12694663 | CGCGCTGAGCTAGGAATAAAGTTCACGTAATCACATGAGAGTGGAGAAAA | | | | Rat | Human | |
| 2599 | cg12737311 | CGAAAACTTGGATATACTGCAGCTTCTTTTGGACTACGGTTGTCAGGTAC | | | | Rat | Human | |
| 2600 | cg12808364 | CGGAGCAGCGTGGTGATAACCAGAGGCGTGAAATTACAAAAGTAAAATTT | | | | Rat | Human | |
| 2601 | cg12813088 | CGCTGTTGCCCTGGGATTCCGATGAATCTCCTGAGGCCTCCCCTGGTCCT | | | | Rat | Human | |
| 2602 | cg12854350 | ATGTGGATCCGAGGCGGAACAGAGCAGTTGTTGTCCCAGAGGATATATCG | | | | Rat | Human | |
| 2603 | cg12854758 | AGCAAAGTGCTGAATGGAATTTGTGCCTACCTCAATAGACATTGGGTTCG | | | | Rat | Human | |
| 2604 | cg12887931 | AAAAGCAGCAGCAGAGAGTTGGAGTCACCTTGACCGCCATGCTGTGCTCG | | | | Rat | Human | |
| 2605 | cg12936001 | TCACCATCCTGGACACACTCGTTTGTCAATGTCCCTCTGAAAATGTGACG | | | | Rat | Human | |
| 2606 | cg12999766 | CCGCAGGAACTTAACTTACACATGCCGTGCCAACAGGAACTGTCCCATCG | | | | Rat | Human | |
| 2607 | cg13023398 | CGTCCGAAACCTCCAACTCCAAGGGTATCAATGATGTTGAAATCAGACAG | | | | Rat | Human | |
| 2608 | cg13078590 | CGAACCATCAGGAAGGCGTCTTCCTTAACATTAATTACAGAAAATGACAA | | | | Rat | Human | |
| 2609 | cg13106512 | CATCCTGGATTTCATAAACCAAAACAAGAGAGCCTGGCAGGAGGACAGCG | | | | Rat | Human | |
| 2610 | cg13123762 | CCCCACCAAAATTTGTTTCAGATCTAATAAAACTGTGCTTAACACACTCG | | | | Rat | Human | |
| 2611 | cg13138986 | ACAAAAACTAAATTAGGCTTCTCCTGTTATCCTACAAATATTTAATGACG | | | | Rat | Human | |
| 2612 | cg13144187 | TGACATCTCAAAGAAGCACTTTGATGCAAGCTTCTTTTGGGCCTGTGACG | | | | Rat | Human | |
| 2613 | cg13158272 | CGTGAATCTATTAATTTAACAGGAACTAGGCTCAGCACATATATTCCTTG | | | | Rat | Human | |
| 2614 | cg13246416 | TTCTTCTGCGATGCTGTAGCCTCCTGGGTGAGCCCGAAGGATGACCTTCG | | | | Rat | Human | |
| 2615 | cg13366833 | CGCTGCCCCAGACTCACAGAGTCTTGGGCATCTCGTCCTCCATGGCTGCT | | | | Rat | Human | |
| 2616 | cg13466649 | AGGCTGCTGAAACCATCAATCTTGCTGGCTCTTGTAGAGGTCAAGAGACG | | | | Rat | Human | |
| 2617 | cg13502730 | TTAGTTCCACGCTATGAGCTAAGTTGATAACATGAAAAGACCCATAAACG | | | | Rat | Human | |
| 2618 | cg13505793 | CGATCAGCCAGCCGTGAACTCTGGGCCGAATTCATTACAGTTTAATAGTG | | | | Rat | Human | |
| 2619 | cg13525872 | CGCTAGGGAAATAAAAGCTTCATCTCAGATTATAGGTGGGTATTTTTGGA | | | | Rat | Human | |
| 2620 | cg13655737 | TGGAAAAGCAGGACACCTTCTTTTTCTTTTTCTTGTTCCTCTGCGCCTCG | | | | Rat | Human | |
| 2621 | cg13768953 | GTCGGCTCGGAACAGATTCGCCTCCATGTGTCTCTGCGTGGTACGTGCCG | | | | Rat | Human | |
| 2622 | cg13777117 | TCTGCGGGAAACATATGTCGGGAGAAGTTCACATTCCGGATTCTGCTCCG | | | | Rat | Human | |
| 2623 | cg13785960 | ACATACCTGGCCATGCCTTTGTGCATATCATTGTGCAGTCGCAACTGACG | | | | Rat | Human | |
| 2624 | cg13895665 | ATACTAATTTCCTCTCCACCTTATCCCCTGAACACTGCAGACCTTTGGCG | | | | Rat | Human | |
| 2625 | cg13899234 | ATGGGAAGTTCTGTGACTGCTGCTACTGTGAGTTCTTCGGCCACAATGCG | | | | Rat | Human | |
| 2626 | cg14002601 | GCCTGGAGTCTTTTAAATATAAGTAACACGCAACCAGTAATAAGCTGTCG | | | | Rat | Human | |
| 2627 | cg14007694 | CGGCTAGTGGACCAGGTGAAGTACATTAGAAACAATTACTTTCCAACTGG | | | | Rat | Human | |
| 2628 | cg14039101 | TCTTCGGTTAACTCCGCTTGTTTCTCTACAAAATGGCGCCGGAGGTCGCG | | | | Rat | Human | |
| 2629 | cg14113335 | CGGTGCGGCACTTTTGTGTTCAGTAGCATGAATCATTAATCATGAGACAG | | | | Rat | Human | |
| 2630 | cg14224932 | CGTGGTAACAGAGTGTTCTTTGTTACCACACACTGCTCTTTGTTATCACA | | | | Rat | Human | |
| 2631 | cg14292527 | CGCCAGGCCTAATCCCTACACAGTGGAACAAAGCCCAGCAAAGCCTTTGA | | | | Rat | Human | |
| 2632 | cg14428543 | CGCACATGCGCACAGAGCACTCAATCTGACACCCCTCGCCGGGGCCAAAA | | | | Rat | Human | |
| 2633 | cg14467140 | CGCTTAGGCCCCTCACCAAACTCTCAGTAACCATGGTAACTGTATACAAA | | | | Rat | Human | |
| 2634 | cg14469373 | CGGGCGGACACCGAAACACGAACAGGAAGAAACCCCCTTCATAAGGAGTG | | | | Rat | Human | |
| 2635 | cg14550429 | CCGAGGTAGGGCACCACTCCCAGGGAGTTGGGGCTGGACACGGAGTAGCG | | | | Rat | Human | |
| 2636 | cg14577476 | GGGTGGCCCCAGGATATCAACAACATTAGCATAGCCCTCCCTCCCCAGCG | | | | Rat | Human | |
| 2637 | cg14628884 | CGCGATGTTGTGATGAAGGTGAAAAGATCGCTCTTAATTAGATTAAAAAC | | | | Rat | Human | |
| 2638 | cg14644523 | CACTCATAAATTTGGCTTGATGACCTCCGGGAGGTTAGTGATGGAAGCCG | | | | Rat | Human | |
| 2639 | cg14665342 | CGTGTGGCCTGCTCTCATGGAGAATGACTGTGTCATTATGAAGGGAGAAG | | | | Rat | Human | |
| 2640 | cg14720152 | CGGATTTAAACCACTAGACAAGCTATTAATTTAGCTAGGCTACCCAGGGT | | | | Rat | Human | |
| 2641 | cg14783231 | ATTGTATTTGCATTTTAATTTTCCACATAAACAAAGGCCCAGAGTACTCG | | | | Rat | Human | |
| 2642 | cg14817764 | TCCGTTCCGTTTTGGTCGAACCCAAGCTGATGTCGCTGCTACTGGCCACG | | | | Rat | Human | |
| 2643 | cg14870509 | CGGCTTGATATTTCCGAAGAATATAGTGGGCTTTATTAGCACCAGTTTCG | | | | Rat | Human | |
| 2644 | cg14980868 | TCCATCTGCTGCTCTGACGCCTAGGAGCAGTTATTCCACAACTGTCTGCG | | | | Rat | Human | |
| 2645 | cg14988533 | TACAGCTTTTTGGGAAGCTGTTTGAAAATTAAGCTTGGATCAGTTACTCG | | | | Rat | Human | |
| 2646 | cg15008665 | CGTGAAATACTGAAATTTGCCCAGTACCTTCTCAGACTATTAACAGGTTC | | | | Rat | Human | |
| 2647 | cg15036140 | CGGCGCTCCCTGCACAGGGTTTCCTGTCACCCTCGCAATGGGCCGGACCA | | | | Rat | Human | |
| 2648 | cg15051960 | TCTTTTGCACAATCACGCCCACATTTGGGGTTGGAAAGCCCTAATTACCG | | | | Rat | Human | |
| 2649 | cg15055738 | AAACTTGCTGTGGGTGACCATGGCGCACGGAGCCAGCGTGGATCTGCGCG | | | | Rat | Human | |
| 2650 | cg15060871 | CGCCTGTTCCCCATGGACCTGAAGTTGAATGCACACCTTGGCTCTGAACT | | | | Rat | Human | |
| 2651 | cg15112657 | GTGAATATCCTTCTCCCTCCTGATGCTAAAGAGATTGCCACTCGCTTCCG | | | | Rat | Human | |
| 2652 | cg15159458 | AGAAGTAGCCTTGAGGGGCCCACCATATTGGACATAGAAAAATTTCATCG | | | | Rat | Human | |
| 2653 | cg15316343 | CGGGAGTACGTGTTAGTGGGAGCAGATGGCTCCAAGCAGAAAATGCCTGC | | | | Rat | Human | |
| 2654 | cg15353421 | CGCAAGGGAAATTATAAAAAAGTTCATGTTCACGGTTCTCATCCACATGA | | | | Rat | Human | |
| 2655 | cg15471499 | GCGCCCGATGTAACTGTAATTACTCCATTGATATTCCTCACACAATAGCG | | | | Rat | Human | |
| 2656 | cg15574290 | CATGTTTCTCTCTATTGTGCCATCATAAAAAAAACTCCGCTGGTGACTCG | | | | Rat | Human | |
| 2657 | cg15591276 | TTTCATTATATCAATCACATTTAATTGGCAATTTGTACGAGCAATGACCG | | | | Rat | Human | |
| 2658 | cg15598853 | CGCCTCTCTCAGGGAGATCTGGAGAAATACGCAGCACAGATGGGCCGAGC | | | | Rat | Human | |
| 2659 | cg15719056 | TTTTACTGCACTGCTCCCTAGTATCTCAGATTCATCTTCCCTGACGCACG | | | | Rat | Human | |
| 2660 | cg15830792 | CACCAGGTTGAGGGGTCCTTCCATTACTTCCATAGACCCCGGGGTAAGCG | | | | Rat | Human | |
| 2661 | cg15861063 | TGTATTTCTGCAAATGTGTGAAATGCAAAATGTGCCCTCCAGCAAAAACG | | | | Rat | Human | |
| 2662 | cg15874422 | TCTCGTAGCCCTTGTAGTTCTTCATGTTCAGTGTCATCTTGCAGGTCTCG | | | | Rat | Human | |
| 2663 | cg15931876 | CGGATGTTGCTCCCCTAGATTGCCCGTGAGGCAGAGGCTGCCATCTACCA | | | | Rat | Human | |
| 2664 | cg15938989 | CCAGACCTTCAGCTGCGAAGAGTGCCTGTTCAAGACCACACACCCTTTCG | | | | Rat | Human | |
| 2665 | cg16101912 | CGGTGGCATGTTTTAGATGACATTTCAATTGTACGAGCAATTTGCATTCT | | | | Rat | Human | |
| 2666 | cg16197002 | AACAAGTACCTGGTGGAGGATGTGCTGCTGCTCAACTTCGAGGAGATGCG | | | | Rat | Human | |
| 2667 | cg16273620 | CGGCGGCAAATGACACTCTCGGAATCTTGACTCCAATCTGATGCCTGGTC | | | | Rat | Human | |
| 2668 | cg16321970 | TTCACCAAAGAAACTTGCGTCCATTGGAGGTAAAATCTTGGCTAGGCTCG | | | | Rat | Human | |
| 2669 | cg16367989 | CGAAGTTCTTGGTGAAGACCAGGGTGACCAGCAGGATGGGCACCAGCACG | | | | Rat | Human | |
| 2670 | cg16434657 | ATTGTTTACTTATCTGCGGGAAACATATGTCGGGAGAAGTTCACATTCCG | | | | Rat | Human | |
| 2671 | cg16528216 | CGCCCAAATCAATGTGGTTTCTTTGGAACATTTTCAGCAAAGGAACGCAT | | | | Rat | Human | |
| 2672 | cg16549948 | CAGCTCACTGGGCTTGAGGTTGGGGTCTATGATGAGCTCCCAGCTGTCCG | | | | Rat | Human | |
| 2673 | cg16589235 | CGGCCAGACGTTGGAGGCAGCTAAAGCCACATCAAAGCTTTCCTTGAAAT | | | | Rat | Human | |
| 2674 | cg16590183 | CGGGAGCAGAAATTGCCTGTCCGGTTTTATGATCTGCTCAGCCTGAGCAG | | | | Rat | Human | |
| 2675 | cg16642472 | CGGAACCTCACTTACATGGTGACCCGCAGGGAAAAGATTAAACGGTCTGT | | | | Rat | Human | |
| 2676 | cg16679083 | CGCCGGTGCTCATCTCCATCCTGCTCGGCGCCAGCTTCAGCCGCTACCAG | | | | Rat | Human | |
| 2677 | cg16680249 | CGCCGAGTTTTATTCTGTCCTGTGGCTCAGAGTTGAAGTTCCCCAAGTGA | | | | Rat | Human | |
| 2678 | cg16694303 | ATGATTTCTGTCAAAATCATCTTTACCACAATTATAATATGTTAATTACG | | | | Rat | Human | |
| 2679 | cg16723516 | AGCACGCACAGCAGAGCCCTAGTCGATAAGAAGTTGCAAATCAGTATTCG | | | | Rat | Human | |
| 2680 | cg16725457 | CGACATCAGTGGCAACGTCCTCGCTCTGATCTTCAACCAAGGCATGATCT | | | | Rat | Human | |
| 2681 | cg16770315 | CTGGGCGGCCGCTCTGCAGGAAGCAGTAACCGAGACCCTGTCTGACTACG | | | | Rat | Human | |
| 2682 | cg16785087 | CGCTTATTAAAGTCAAGCATGTGTCTTTGTTTCATGTTGCTAATGATGTG | | | | Rat | Human | |
| 2683 | cg16821706 | CGCCAGTCTGTGATTTAGCTGTCGCCAGACACCCCATGTTTTTTGTGAGA | | | | Rat | Human | |
| 2684 | cg16966998 | CGTACTGGTTTCTGCAGTCTCGTACTCGCTGTCTCTGAGAAGCTCAAAGA | | | | Rat | Human | |
| 2685 | cg16983152 | CGCTCAGCCGCCGGAATCGCAGCCACTGCCGGCTTCACAGACGCCTGCCT | | | | Rat | Human | |
| 2686 | cg17012009 | CGTCTTCGTTGCCATAGAAACCAAGGCTACCTTTGGGCTCATCCGGAGTG | | | | Rat | Human | |
| 2687 | cg17092784 | GTTCATCCAGAGCAGCTTCCTCCGAGGCCATGGATTACATTAAAAGAACG | | | | Rat | Human | |
| 2688 | cg17219049 | AGTTTGGGGCCCCAGGCTTTCACTCCCATAATTCTAACAGGGCAATCACG | | | | Rat | Human | |
| 2689 | cg17225945 | TGTTCAGGCTACTGTTGATGCAAATACTACTGCCATTCGCGGCAGCAGCG | | | | Rat | Human | |
| 2690 | cg17254187 | GAGCAATGTTCTGTGATGGTGCCCGAGATGCGTGGACTGAGCCCAGACCG | | | | Rat | Human | |
| 2691 | cg17322734 | ATTTCACAGTACACGCTTTCAAAAGCAGTCTCAGGTGGCCTGATGAAACG | | | | Rat | Human | |
| 2692 | cg17326640 | GCATAGCCAATAGCTCTGGGGCTCCTGCTACTGCGCGCTGATTGTTACCG | | | | Rat | Human | |
| 2693 | cg17489004 | CGGACCCCCTTGTAGATATAGAAGTACAGCTCCCGGCCCACATTGAAGCA | | | | Rat | Human | |
| 2694 | cg17502250 | CGTGACCTCTATAATGCTTCTTAAATTGAGGACAATGATGGACCCACCAA | | | | Rat | Human | |
| 2695 | cg17613601 | CGGACCATTAGTTACATATTTGAACCAATAAGGAAACACTTACCGAGCGC | | | | Rat | Human | |
| 2696 | cg17655329 | CCTGTGACACCAGCTAAATTAATCACTCACTGCACTCAGGGAAGTCACCG | | | | Rat | Human | |
| 2697 | cg17760546 | CGGTTTGTATTCTCCTACGGCTTTTACAGAAGTTGCAGTGATCCAAAGTC | | | | Rat | Human | |
| 2698 | cg17876669 | ACTTTACATTTTGGAGACAAACCTGTGTGAGTTTTTTATTGGTACAAACG | | | | Rat | Human | |
| 2699 | cg17876895 | ATCTGCACCCTCAAGGTGTTCCAGGACAGCAAGGCCTGGGAGAACTTCCG | | | | Rat | Human | |
| 2700 | cg17915458 | CGTCCTCTACCTGAACCCCGATGGACTGGAATTTGGTAGCGGGACTGGGC | | | | Rat | Human | |
| 2701 | cg17995270 | CGCCATGGACCTGCTGCGCATCCCAGGCCTGCTCATGTACATGATCCGGC | | | | Rat | Human | |
| 2702 | cg18111337 | GGACACCCTGCTGGTGTCTAGGTAGGAGTTGGAGTGCGTCCCGGTCTCCG | | | | Rat | Human | |
| 2703 | cg18120532 | CGGAATTATGATACAGTGGAGCCTTCACGCAATTCTTTGTTGAAGAAGTT | | | | Rat | Human | |
| 2704 | cg18141729 | CGCGGGTCACAGATTGAATCAATTGTCCAATCTTGTGAAAGTCATATCCT | | | | Rat | Human | |
| 2705 | cg18147524 | TAATGAAATGAAGCACAGATCTAAAGATTACTCAGCCCCTTTGTCAGTCG | | | | Rat | Human | |
| 2706 | cg18166816 | CGGGGGTTTATTTGATCTCACATTAACCAAGGAGGAGAATGTGAGAAAAG | | | | Rat | Human | |
| 2707 | cg18280269 | TTAATTCCTTTGTTAATGTCTTTATGAATGCAATTAACACTTCATTCACG | | | | Rat | Human | |
| 2708 | cg18425696 | CTCTCGTTGCCACGTAGCAGGTCGCTCTGGGGCAGGGACCACAGGAACCG | | | | Rat | Human | |
| 2709 | cg18462586 | CGGACCTCCTTGCTAAGCACCACATAGGAGAGGAAGATGAAGGGGCCCTG | | | | Rat | Human | |
| 2710 | cg18465972 | CGGCTCTTTCTGGGTTATCAGGCGCCAGTCCAAGTCCTCCCAGGCAGCCA | | | | Rat | Human | |
| 2711 | cg18466140 | TAGGCCGCAAATCGCGCTGAGCCTCCCTAAGTGACAGCGAGAGAATCGCG | | | | Rat | Human | |
| 2712 | cg18484878 | CGCTCCTTGCTCTGTAAAGTGGATGTCAGGTGGATCTATGTTTCTGAAGG | | | | Rat | Human | |
| 2713 | cg18496572 | CGTCACAATAAGTGGTAGTAATAGAACCATATACAGGATTGTAAATACAA | | | | Rat | Human | |
| 2714 | cg18775165 | AAAAGAACTTCAGTGGCGTAAACACCGTTTGTGCTGTGTCCCCAGCCTCG | | | | Rat | Human | |
| 2715 | cg18856868 | TTGAACTGATTTCACAAATACCCTTTCATTTCACTACTTCATTATGCTCG | | | | Rat | Human | |
| 2716 | cg18862976 | CGCCTGACTTTTACCAACTTCAGCACGCCAGCTGAGGCCTATCCCAGCTG | | | | Rat | Human | |
| 2717 | cg18961099 | CGTCCCTTTCAAAGTCTCCGAGTTCTTCATGCTTGTTGTTGAGATTTGAG | | | | Rat | Human | |
| 2718 | cg18987102 | ACAGCTCACTAATTAATTAGCGCTTAAGTACTTAGCATCCTCCTGCATCG | | | | Rat | Human | |
| 2719 | cg19067187 | CGTTTGATTAGTGCTACAAAAATTTAATTAGGTTGGCTAATTACTTGACA | | | | Rat | Human | |
| 2720 | cg19078816 | CGGTTTGGTTTCTTTAATCATTAGTTGCAGCGATGCGCTGGCAAACCCAG | | | | Rat | Human | |
| 2721 | cg19091932 | CGCGGATGAAATTAGCACTTCCTGCTATGTCGGGCAGATAAACTTTGCTG | | | | Rat | Human | |
| 2722 | cg19093840 | CGCAAGTTGACGGAGGCTTTTGATTGAATTGACTGATTGCCTCAGCTGCA | | | | Rat | Human | |
| 2723 | cg19117941 | AGATCTCGCCTTTCCAGATGCAAAAGTTCAGCCCCTCTGATGTCATGACG | | | | Rat | Human | |
| 2724 | cg19142100 | ATGATCCTGTCTGGCTTGCCTGCCCCAGATTTTATTGATTACCCTGAGCG | | | | Rat | Human | |
| 2725 | cg19169532 | CGTTCATGGAAGTGTGAAATGGCAAACAGGTGAATGTCCAATTACACTGT | | | | Rat | Human | |
| 2726 | cg19171923 | TTTTGACAGCGATCATTTGCTTCCTCTGCTGGATGATCTCTACCAACTCG | | | | Rat | Human | |
| 2727 | cg19239233 | TTCTTATTCAAAACAGGCCCACAATGGGCTTCACAGTGCGCCTCGCCACG | | | | Rat | Human | |
| 2728 | cg19261448 | CGCCTAAGGAGGCATAAAAGGCCACCAGGAAATAAATTAAAATTAAAATA | | | | Rat | Human | |
| 2729 | cg19302474 | CCCATCCTGCTGGGTCAAGTAGGATTCCATTTCAATTTCTTTACACAACG | | | | Rat | Human | |
| 2730 | cg19355190 | GAGTTACTGGGATTTTGTCTACGGCCTTGGCGGTCATCATTTGCTCCTCG | | | | Rat | Human | |
| 2731 | cg19367422 | CGAAAGGCAAGAGAATTTCTTCATTGCCCTTGGTGAACCGAAATGGATGG | | | | Rat | Human | |
| 2732 | cg19510597 | GGGCATCAGGTGGTAGTTCATGGCGTCCAGCAGCAGCTTCTGGCAGACCG | | | | Rat | Human | |
| 2733 | cg19641802 | ACAAATGTGGCTGCTGGGGAAAGTGACACGGGCTCCAGGTGATGGATTCG | | | | Rat | Human | |
| 2734 | cg19702785 | CGGGCTTCTTCCTGAGCCTGCTGCACTTCTACCGCACTGGCCACCTGCAC | | | | Rat | Human | |
| 2735 | cg19723657 | GCATCTACTGCAGTGAAGGCACTTGTAGCATTATGGTGACAGCTGCCTCG | | | | Rat | Human | |
| 2736 | cg19755091 | CGGCACTTGCAAGATATGGTTGCCGTAGCAACAGGCCTTCTAATCTGGTA | | | | Rat | Human | |
| 2737 | cg19773399 | GGACCTCTATTGCTATATCTTGGAAGCCACCTTCTTGATTTCCACTGACG | | | | Rat | Human | |
| 2738 | cg19778148 | CGGTTGTAATTCAAATCTAATTCAGAACATCTTAGCACCGGATCTGTCAG | | | | Rat | Human | |
| 2739 | cg19795032 | TTAGCAAAAATCCTATGAAAGACTGTTGGGAACAGAGCAGCCCGTAAGCG | | | | Rat | Human | |
| 2740 | cg19890585 | ATGGTGAGCTTCTCCGCCAGCTTCTGCTGACTGGGCTGCAGCACTGAGCG | | | | Rat | Human | |
| 2741 | cg19897940 | CATTGTTCCCGGCTAGCTCTTATCTCCCAGGAGCAAGTATCCTGTGTGCG | | | | Rat | Human | |
| 2742 | cg19945840 | GCCAGGCCAGCATGGCCAGCACCTTGGCCGTGAGGTTTTCGTAGGCGTCG | | | | Rat | Human | |
| 2743 | cg19950962 | CGCGCCCAGTAGAATATTGTATTAGTTGGTTGTGAGCTGTGTTCTCGTCA | | | | Rat | Human | |
| 2744 | cg20093263 | CGTTCTGTTCCAATAACCTGAGAAAAGTTTATGGACATGCCTTGCTGCCT | | | | Rat | Human | |
| 2745 | cg20144788 | CGCCGGGTATTTTCCCAGGTAGCTTAGAGGGGAACCTTGTAAGACATGGA | | | | Rat | Human | |
| 2746 | cg20161627 | CGTCCCAGGGCAAACCGGATGTGAAATAGGCGGTGACTTCATGCCAGAGA | | | | Rat | Human | |
| 2747 | cg20206504 | CGCCGGATTATACGTGGCCGATTCCCAGTGATTGCCCTGCTACACAAAGC | | | | Rat | Human | |
| 2748 | cg20264796 | ATCATTAAGGTTCAACAGCATTCAGAAGCTCAAATCCAATCAGTTTGCCG | | | | Rat | Human | |
| 2749 | cg20538492 | CGGCAAACTGAAATTAACAGCTACTGGAGCGCATTAATAATCTGCCTCAC | | | | Rat | Human | |
| 2750 | cg20593457 | CGAGGGGTTTGCTGTGGAATAAAATTAGATTCCAACATCTGCCACCATTA | | | | Rat | Human | |
| 2751 | cg20656650 | CGGGAAGGGGTCCTTTGGACAGGTGGCCAAGTGCTTGGATCACAAAAACA | | | | Rat | Human | |
| 2752 | cg20677676 | TATCAGCGTCAACTATCGCTTCCTCTGCTGGTACACACGGCCTGATGCCG | | | | Rat | Human | |
| 2753 | cg20728571 | ACAGTGGGCGTATTTTATGGAAAGCAGCCGACAATCAGTGTTTGCAGACG | | | | Rat | Human | |
| 2754 | cg20757950 | CGCCATTTGTCAAAGTCAACCTTCTGATTGCTTTGGACCTACTGCTATCC | | | | Rat | Human | |
| 2755 | cg20829742 | CGAAATGAAAACTTAATTCAATAATAAATGAGCTTCAGAGCAGCACAAGA | | | | Rat | Human | |
| 2756 | cg20844381 | AAAAACATTTATTAATATGCAAATTCTAAGTGGCAATCTGGACCCAGTCG | | | | Rat | Human | |
| 2757 | cg20850964 | CGGCGACTCCGTTCAATTTTGGTAACAGTCTGGCAAGTGCAGGTAGATAC | | | | Rat | Human | |
| 2758 | cg20860111 | AGATATAAATAACATTATCTGAGGAGGGACATTCCTGCCGGGGGACATCG | | | | Rat | Human | |
| 2759 | cg20951495 | CGAACCACCACAGGCCTCTCACTGCCAGCCACGAAGCCAAAGCCATATAT | | | | Rat | Human | |
| 2760 | cg20984125 | CGGAATTTTGTCCTGGATAACACAGATCGCAAGGGTGTCCATTTTCTGAT | | | | Rat | Human | |
| 2761 | cg20989633 | AATATCAAAGCAGGCAATGCATGTGTGGCTATGCCTCACAAAATAGGTCG | | | | Rat | Human | |
| 2762 | cg21026697 | AGTTAATTTGGCATCTGTATTTTTTACAACCCCGCACACGTAGCCCAACG | | | | Rat | Human | |
| 2763 | cg21057587 | CGCTCCTGGCCCCGGTGATTGGCTGCTCAGCTCTGACCCCACTTGGGCTG | | | | Rat | Human | |
| 2764 | cg21098898 | CGTATCTTTATTTCTAGCACATTAACAAAATATCACAAATAAATTGTCCG | | | | Rat | Human | |
| 2765 | cg21115959 | TAAACTCTCTCCTGTCTTGGACGGGGTCTCTGAGCTTCGTCACAGTTTCG | | | | Rat | Human | |
| 2766 | cg21119395 | CTGGGACAGTGTGTGCTTGCAGGTGTCTCTCAGATACGTGCTACCTGTCG | | | | Rat | Human | |
| 2767 | cg21219688 | AGTTACCGCTCTCAAACCTACCAAAACTACAAAAACTTTAATAGCAGACG | | | | Rat | Human | |
| 2768 | cg21229874 | CCAATGAGATTTGAAAATGGCCTTGAGCCGCAGCCTCCTCTCGGGTCACG | | | | Rat | Human | |
| 2769 | cg21308134 | CGCCATTAAGTCCCGATAGACAATACCAGAGTGGTTTTCTTTCAACTGTG | | | | Rat | Human | |
| 2770 | cg21332416 | TCCAAGAAGTACAACATCCTGGGTACCAATGCCATCATGGATAAGATGCG | | | | Rat | Human | |
| 2771 | cg21508838 | CGCGGGAGACCAAGGTAATGATGATGTCATGAGAAATCTCAAGTACATGG | | | | Rat | Human | |
| 2772 | cg21523490 | CGCGCGGGGACCTAATCCATCAAATTGTCTACAAATTGTGAAGAAAATTC | | | | Rat | Human | |
| 2773 | cg21583851 | TGTGTTACAGAGTACAGTTGCTGTGTTGCAGAATGTTAAAAACCTCAACG | | | | Rat | Human | |
| 2774 | cg21603943 | CGTCCGGTCTGAAGTTTCTCCGAGTAAACAAGGATGAGGGACAAAAGCCA | | | | Rat | Human | |
| 2775 | cg21658655 | CGGCATTGTGGTTCATGTTTGAAATTACAGATTTTAAATGCCATGTTCAT | | | | Rat | Human | |
| 2776 | cg21663142 | AACCATGTAGGCCCTGCAAAGGCTACCAGTCCATCTCTCCGTCTTCGTCG | | | | Rat | Human | |
| 2777 | cg21674021 | GACTGTTAGCAATCAGAATGCTTTTTATATGGTGTCTTTCTTTGGAAACG | | | | Rat | Human | |
| 2778 | cg21685750 | GAGCCCAGTGGGAGGTGGAGCGGGCGGAGCTGCAGGTAAAGACCCTCCCG | | | | Rat | Human | |
| 2779 | cg21694863 | ACTTTGAGACATGGCTTGATAACATTTCTGTTACATTTCTTTCTCTGACG | | | | Rat | Human | |
| 2780 | cg21743335 | CGGCTAATAAGTTGTCTGACAAGATAGCCACTTTGTACCTAATTGTTCAG | | | | Rat | Human | |
| 2781 | cg21760034 | CGCACGGACATCCTGTGCAACCTGCCCAGCTACAAGGCCAAGGTGAGAGA | | | | Rat | Human | |
| 2782 | cg21878650.1 | CGGCTTACTTTAGCTACTTACGCTGGACTGTCACAGTGTCTTAGGGATGA | | | | Rat | Human | |
| 2783 | cg21900509 | CGACCTGGAGACAGCTTTTGTGTTATCCTGTCAATGGTTATAGAAGGATA | | | | Rat | Human | |
| 2784 | cg21907579 | ACTCGTGACGTCTCCAAGTCCTATAGGTGCAGCGGCTGGTGAGATAGTCG | | | | Rat | Human | |
| 2785 | cg21917735 | TTAATAAGAATCTGGGAAGTATCTGTCTTTGCCGGCCGCTTTCATTTGCG | | | | Rat | Human | |
| 2786 | cg21926612 | TCTGGATCAACCTGGGCACTACGAGGGGTTGAATTTCTACCATTATCGCG | | | | Rat | Human | |
| 2787 | cg21940324 | ACTATATCCGTGCATCTTTTTAAGCAGATGAGGTCCAGCGTGCTCCAGCG | | | | Rat | Human | |
| 2788 | cg21956876 | CGCCTTAAAAATACGAAAATAAATTTTACAAGGTTTCTGTTTTGTGTGGA | | | | Rat | Human | |
| 2789 | cg21960684 | CGGCCCTGGAGGAATTCCCAGTGTAAACAGGGAATTCTCTAAGGGAACCC | | | | Rat | Human | |
| 2790 | cg21993706.1 | TCTCTGACAGATGTGTGCCCAATCACTGTGAGCATGGTGGAAAGTGCTCG | | | | Rat | Human | |
| 2791 | cg22000069 | GAGATGGCGGATCCAGCCGAATGCAGCATCAAAGTGATGTGCCGGTTCCG | | | | Rat | Human | |
| 2792 | cg22044223 | CGGAAAGTAATTAAGGAAAACAAGTTTGAAACTGCTGACTTCAACCCTTT | | | | Rat | Human | |
| 2793 | cg22048239 | ACTTAAAACTGGGGTCAGGTGGAAGTCTCACAGAGACCACGTCTGTACCG | | | | Rat | Human | |
| 2794 | cg22097793 | TGCATCTTCCCTTATTGAAAGGTTATTTTATGCAAGTAAACAATTCATCG | | | | Rat | Human | |
| 2795 | cg22110517 | CAAGTTTACTTCATGACTCTGAACCGTAACTGCATCATGAACTGGTGACG | | | | Rat | Human | |
| 2796 | cg22171829.2 | TCACCAAAGTCCAGTAGCTGCTTCATGGACAGCGGGGACGGGCTGTAGCG | | | | Rat | Human | |
| 2797 | cg22177193 | CGCAGTTTTGAAGAAGTATCAAAGTAGGAGAAGAGTGAATCTAGCTCATC | | | | Rat | Human | |
| 2798 | cg22373432 | CGCAAAGGGGGAGAATCACCCGTCTCCTAATTTTAACCAGTACGTGAGGG | | | | Rat | Human | |
| 2799 | cg22375533 | TATCACTTTCTCTTCTTAAAGCAAAGAGCAAATCGCCTGTAAAATCTACG | | | | Rat | Human | |
| 2800 | cg22450471 | CATATGTCCAAGCCTGCGGTGCTGGGACTGAGCATGATTATACCCCGACG | | | | Rat | Human | |
| 2801 | cg22493172 | GGGCGGTGCAGCATGCGGCAAAGCTCTTTGCATAAATTATGCTCATGACG | | | | Rat | Human | |
| 2802 | cg22533021 | GCCCACCGGTGGGGACAGTGTGGCATTTCTTATGCTCCAGCAGCTGTTCG | | | | Rat | Human | |
| 2803 | cg22598501 | CCTCTTGTCTCGACAACTGTAGATATTTTGTTTTCCTTCTTTCTCTCTCG | | | | Rat | Human | |
| 2804 | cg22684134 | CGCAGCACAAGGCAATTTTAAAATCCATTCTGTAAAAGCAACACTGTTTG | | | | Rat | Human | |
| 2805 | cg22689219 | AATGTAAAATCCCATATGCTGTTATACAGTATGTTGCAATCTGTTTTACG | | | | Rat | Human | |
| 2806 | cg22738179 | GAAAGCCCTACGGCGCACTGCAATGTGCTGAACTGGGAGCAAGTGCAGCG | | | | Rat | Human | |
| 2807 | cg22900813 | CGCATGTCAGAGAGGTTGCAGGCTGTTTTCAATTTGTCAGTTTGTGGATG | | | | Rat | Human | |
| 2808 | cg22935821 | GCAGGAGGCAGCTGCATATCTGCGGTCCCGACCACAATTGCTGGGAGTCG | | | | Rat | Human | |
| 2809 | cg22944122 | TCTGGCTCTGACTTTCACTCCGGGGCAACAGTAGCACCAAACCACATACG | | | | Rat | Human | |
| 2810 | cg22949164 | AGGGCTCTGATTCAGAAAGATCATCGGATCCGTCTGAGCTTGGCTGGTCG | | | | Rat | Human | |
| 2811 | cg23045679 | CGTGTTTATAGTACAGTAAGTGGTCTAGCAAAATTGTCCATGTTTCTTTG | | | | Rat | Human | |
| 2812 | cg23046555 | CGCGCCGGAGCGTGACTTTACAACTAATCCTGTTTGTAAGTTATTATTTA | | | | Rat | Human | |
| 2813 | cg23075119 | CGGGGCTGGCTCTGGGCCTCAGACACTCTGGAATCGCCAAGGTCTGAGCT | | | | Rat | Human | |
| 2814 | cg23119788 | CGCCCTTTTCAGGCGCAGACAACAGAACAAAGTATAGAGGAACAACAAAA | | | | Rat | Human | |
| 2815 | cg23126716 | CGCCAGCAGCTGCAACTTGGGGGGCATCATAATCCTGAGGAGCCCCATGA | | | | Rat | Human | |
| 2816 | cg23164054 | AGTGGTAACCATCCACCACTTGAGTCATCTCGGGTCATCACCACAGCTCG | | | | Rat | Human | |
| 2817 | cg23192185 | GTATTCGATTTGTTTGAGTCCCTTATAAAAACACTAATGCAGACCAGACG | | | | Rat | Human | |
| 2818 | cg23194111 | CGGGTGAAGTGTAATTTGTTTTTCTTAAGTTTAATACAGCATGTCAATAG | | | | Rat | Human | |
| 2819 | cg23352950 | CGCCAATCGATTTACCTCCAATGCAGGTGTGCTACAAATCCGCTGGGCCC | | | | Rat | Human | |
| 2820 | cg23442072 | GAGGATACCTCAGGCACTGAGAATGAGGCCTACTCCGTGGGCACTGGCCG | | | | Rat | Human | |
| 2821 | cg23449725 | CGCGCAGAAGCTCTTTGCGCACAGACATCTCTCCAAGGTGACCCATTTGG | | | | Rat | Human | |
| 2822 | cg23478212 | GAATGAGAAATGTTTATATGTTCTTAGTTGCTTTTAAATCTCCCTTCACG | | | | Rat | Human | |
| 2823 | cg23572376 | AAACTTCTGAACTTTTGAGCATCCGTCACCCAGGCGAGGACTTTTTTACG | | | | Rat | Human | |
| 2824 | cg23575948 | CTGCATCTTGATCCTCCTGTACGCCATGTACAAGTACAGGAACAGGGACG | | | | Rat | Human | |
| 2825 | cg23589509 | ATGCATTATACAGCTTGCTGTCTACATTGATCAAATTAATCAGAAAATCG | | | | Rat | Human | |
| 2826 | cg23595572 | CTGGCATGGCTGTGAGCAGACCCTGGCTCGCCTAAGAAATGCCGTGCTCG | | | | Rat | Human | |
| 2827 | cg23681447 | CGGCATTCGGCAACAGCTGTCACCTGCTATAATGTTCTTTAATTATCTTA | | | | Rat | Human | |
| 2828 | cg23691177 | CGGGCTGCATCAGCACTCAAATCTTATCTTGTTAAATCAGACAAATGTAG | | | | Rat | Human | |
| 2829 | cg23925713 | CGCAGACAATGTCCTCTGTGCTGTATTTGCCAATGAAAGTGGACAACAAC | | | | Rat | Human | |
| 2830 | cg23976192 | GGGCTCCATAGCTCAGGGGTTAGAGCGCTGGTCTTGTAAACCAGGGGTCG | | | | Rat | Human | |
| 2831 | cg23977950 | TGCTGCGTTATCAGCCTATTGATCATGTGTCGCCTGTAATAGGACAGGCG | | | | Rat | Human | |
| 2832 | cg24005961 | CGGCGGCGTGGGGTTCGCTGTGTGACACAATTACAACAACTTTGTGCTGG | | | | Rat | Human | |
| 2833 | cg24039358 | CATCAAATTCATTTACGAGGTCCTCCATGGACTTGAAGCTGATTATTTCG | | | | Rat | Human | |
| 2834 | cg24040066 | TAAAATCCATTCTGTAAAAGCAACACTGTTTGCAGAGTTTGAAGAGCTCG | | | | Rat | Human | |
| 2835 | cg24078962 | TTTCGTTTTCAATAGTTATGGTAACAGCATTTCTCCCAAGCACATCTACG | | | | Rat | Human | |
| 2836 | cg24082321 | GTGCTCGTTGAGGCTCATCACCTCGGAGTAACTCTGGCCGATCCAGTCCG | | | | Rat | Human | |
| 2837 | cg24128008 | CGGCGTGTGAATAATTACAAAGTAGCAAGTATTTTTACATACAGCAAATA | | | | Rat | Human | |
| 2838 | cg24143986 | TTCTCTGCAGAATGTCAAACAAAGATCGACACATTGATTCCAGCTGTTCG | | | | Rat | Human | |
| 2839 | cg24230340 | CGGCAAGGGGAGTTTGTTGTTGCCATGGGAAGTCCCTTTGCACTGCAGAA | | | | Rat | Human | |
| 2840 | cg24231456 | CGCCAAATGTCTATGTTCTTGCGCTGGGCTTTCGAGAAATGGTCCCAAGC | | | | Rat | Human | |
| 2841 | cg24243964 | CGCTGGGGTGATTTCTAGCATACTTTATCATGTATCAATATATACTCTGT | | | | Rat | Human | |
| 2842 | cg24257227 | CGCAGCTGCATGGACAGCACGGTGAAGTACATGAATTTCTACAAGATCCC | | | | Rat | Human | |
| 2843 | cg24288746 | CGCTTGTGGCAAAATAGAGGTAACCTTGTTGCAATGCTTTATAAGTTGGT | | | | Rat | Human | |
| 2844 | cg24467057 | TTTGGGAGGTGATTAACAGCAATCACGCTTGAGTTTGACATCCAGGCACG | | | | Rat | Human | |
| 2845 | cg24470284 | TTTTCTCTTCTTGTAATCGCTCCACCAACTGCTGAGGAATATCATGGTCG | | | | Rat | Human | |
| 2846 | cg24545466 | CGTGATCAGGAAAAAGGAAATGAAACCAGAGACAGAGGGAAGCTGAGCGA | | | | Rat | Human | |
| 2847 | cg24545857 | GATTTTTCTTTATCCTCATTAAAGATCCCAGGTGCAGACAAAGCTTTACG | | | | Rat | Human | |
| 2848 | cg24560242 | AACAGCACTTTCCTAAACTATTGTATTTATTAGCAACCTGTTGCCCTTCG | | | | Rat | Human | |
| 2849 | cg24620051 | TAGAGTCAATCAATTATGAAGGGAACAATCTGGCGGCCCCTCGGGGAGCG | | | | Rat | Human | |
| 2850 | cg24688511 | CGGTGCGTAATGGTGTGGTATTAAATTCTAATTAGAGATGCAGGAATCAA | | | | Rat | Human | |
| 2851 | cg24737665 | CGTGATTTAATGGCTAATCTTACACTGCCCCCTGGTGGGCGTCCATAGAC | | | | Rat | Human | |
| 2852 | cg24790812 | CGCTGAAGTCAGTGGTGTTAAACACCAGCTGTGCTGGCAGAGAAGCGTGG | | | | Rat | Human | |
| 2853 | cg24967047 | TCGATGACCAGGTCGATGGGCATCTTTCCCTTGAGGCAGGTGATGTAGCG | | | | Rat | Human | |
| 2854 | cg25000361 | CGCTTCTTTTTTCCTATCTTTTCTTTTTTCCCCACTTTTTCTTCTCTGCG | | | | Rat | Human | |
| 2855 | cg25007568 | CGCTGCGACATCTGCCAGATGCCCTTCAGCGTCTACAGCACCCTGGAGAA | | | | Rat | Human | |
| 2856 | cg25019710 | CGCCTCTGCTTCAATGTGGGCCGGGAGCTGTACTTCTATATCTACAAGGG | | | | Rat | Human | |
| 2857 | cg25028274 | CGCGTGGGGCTCAGAGCAACAGGTGGGAGTTTCAGAAGCATTCTGGTTTC | | | | Rat | Human | |
| 2858 | cg25053457 | CGATCACACTTGCTATGAAAGTGGCCACATACCAAATCATAGTACTACCG | | | | Rat | Human | |
| 2859 | cg25179135 | CGCCAACACATGGCACCCCCAGCCCCTGCCACGCCCAGCAAGAGCAGCGC | | | | Rat | Human | |
| 2860 | cg25231972 | CGAGCATTTAGACACAAGCGAGAGGATCATGGCGGATGGCCCCAGGTGTA | | | | Rat | Human | |
| 2861 | cg25237345 | CGGCTCTTGCTGCTCATTGGGGAGGTGACACAAGTGGGTGATAGTGGCTT | | | | Rat | Human | |
| 2862 | cg25322223 | TACGCCAAAACCCACATGATCTGTAACGTCAGCATGCGTATTTGCATACG | | | | Rat | Human | |
| 2863 | cg25431896 | TTTTATGTACCGAGTCTCTTATCCGTCTCTCCTAATGACTTCCCTTAGCG | | | | Rat | Human | |
| 2864 | cg25558063 | ATATCAAGAGGATGGATTCGACTTAGACTTGACCTGTATCCATTTCTGCG | | | | Rat | Human | |
| 2865 | cg25592264 | GTCAAACTCGAGGTCTGGGTTCTTGCAGATCATGCCCGGCATGGTGGACG | | | | Rat | Human | |
| 2866 | cg25670428 | CACTGGAGCCCTGCCTCTTCCTGGCAGGGAGCATCGAGTACAGCTGTCCG | | | | Rat | Human | |
| 2867 | cg25776150 | CGGTTGATGTGGTTAGCTAACTGCCCTGGGCCCTGTATTGGTTTTGCTCA | | | | Rat | Human | |
| 2868 | cg25836301 | CGGCTAGAGCAATTTGTCATAGAATCTGGGGGGCTCATTTTTCCGGCCAA | | | | Rat | Human | |
| 2869 | cg25843357 | CGCCGCCGCCGCCGCCGCACTATAATTTTCCAAACAGGATCTTGCAATCA | | | | Rat | Human | |
| 2870 | cg25889693 | CGTCCATCAGCGACGGCGGTAATTAGGGCTTTCCAACCCCAAATGTGGGC | | | | Rat | Human | |
| 2871 | cg26039561 | CGCTGCTGGCTTTGTCTTAATTAGGCACTATTGATGGAAACAGGGAGGGC | | | | Rat | Human | |
| 2872 | cg26041637 | GGGGTATTTTAGTGACGCCTGGAACACGTTTGACTCCCTCATCGTAATCG | | | | Rat | Human | |
| 2873 | cg26101296 | TTGTTTCCAGGTCATATTTAGGGTTCACATATTTGCATAAAAGCATTTCG | | | | Rat | Human | |
| 2874 | cg26112867 | CGAAGGCGAAGCCCCGGCTCTGACCTGGAGACAGCATTCAGATACAGACG | | | | Rat | Human | |
| 2875 | cg26138622 | CAATTTCTCCCTTTTTAACACCAGCATAATTGACTTTGTACCATCTGCCG | | | | Rat | Human | |
| 2876 | cg26227545 | GCCTTGAAATTAGCGAGGTAGAATGCTTGGGCTAATTATGCATTCAAACG | | | | Rat | Human | |
| 2877 | cg26294925 | CGAACCACTTTCTGGGGATTAAGCGCATGGCTAAGCCTACTGGACCAGAC | | | | Rat | Human | |
| 2878 | cg26307512 | CGGTTGAGGTCACTAAGCATCTGGTTGATTTGGGAAGTGTTCTGAAGCAC | | | | Rat | Human | |
| 2879 | cg26417435 | CGCCGGGGCCGGAATGGCACAAAGTCCTCATCATTGGGGTCATCTACCAT | | | | Rat | Human | |
| 2880 | cg26496365 | CCTTCTAAAGAACCAAAGCCTGTTGGCTTAAACAATAAAGAGCGTTTCCG | | | | Rat | Human | |
| 2881 | cg26499416 | GCAATACAGCTACTCACGTAGCTAAAGGGGCCTTCATGTCCTTACTTTCG | | | | Rat | Human | |
| 2882 | cg26522240 | CGCCCTGAGCGTGAGCATCATGTGAAAACGTGATCGCCAAGTTTCTCTCT | | | | Rat | Human | |
| 2883 | cg26621696 | CGCCTCCCATGGACCCTTCTAACTTTGTCACCATGATGGGCATGGGGGTG | | | | Rat | Human | |
| 2884 | cg26625831 | CGGATCCTGGGAGCTGTGGCCAAGGTCAGAGAAGCCTTCCAACCTCAGGA | | | | Rat | Human | |
| 2885 | cg26674451 | CGTCAGCAGCAAAGTGGTTCGTTACGCCTGATTGGTCTAATTGCTTTGAA | | | | Rat | Human | |
| 2886 | cg26747497 | CGTCTGCGCCCACATCGTTGCTGACCTTGCAGAGGTAGTAGCCACTGTCT | | | | Rat | Human | |
| 2887 | cg26768458 | GGGAACCAGCCACAATTGGCTATATTAATAAATAACTTTCCTCACAGTCG | | | | Rat | Human | |
| 2888 | cg26770198 | CGGTTCACTCAAGTAAAGCAGTTTGTATGGTAATTAAGGGAGCATTTTTC | | | | Rat | Human | |
| 2889 | cg26813228 | CGGGCATATGACCACAGTATCACAATCAAGAAATTTTAAGACAACATTAA | | | | Rat | Human | |
| 2890 | cg26843878 | CGTGCTGCTGCTGCTGCAGGCCATCCTGGGCTCCATGGTGAACGCCTTCA | | | | Rat | Human | |
| 2891 | cg26914546 | ACATTTAAACAAAGTGACACAGATCATCAGCAGATCAACTGGGAATGACG | | | | Rat | Human | |
| 2892 | cg26973329 | CGGCATATGGAGATTATATACTTTTTTGAATATCGTTTCCCAAAGCCTGC | | | | Rat | Human | |
| 2893 | cg26976812 | AATAAAGCAAATTATTCCCGTCTTCAGAAACGATCTAGACTAATTAGTCG | | | | Rat | Human | |
| 2894 | cg26984049 | CGCAGCAACAATAGCAGAGGAACAGCTCTGCCTGGTGACGTAATGGCTGG | | | | Rat | Human | |
| 2895 | cg27050569 | CGACAGAACTGATATTTTATATTGAAATGGATCCTCCAGGTACTTGGGAG | | | | Rat | Human | |
| 2896 | cg27075086 | AGTCAAGGGAAACCTCATTAGAGAGGATTTTCTTAGTTAGCAGGGCTTCG | | | | Rat | Human | |
| 2897 | cg27151981 | CACAGTGATGCACAAAGAGGTAACTTCTGTAAGGCTCTCTGATATAAACG | | | | Rat | Human | |
| 2898 | cg27152481 | CGGCAGCGGCGATGGCTACAGAAGGAGGGAAAACCTCTGAGCCAGAGAAT | | | | Rat | Human | |
| 2899 | cg27171735 | GAAACACTTATTTTCGGAATTTTATAGCTCAGCCACAAAAATTCACTCCG | | | | Rat | Human | |
| 2900 | cg27196467 | CGTCATTGTGCTTTTTGTTAAATTATTGGAACAGTTTGGGCTGATTGAAG | | | | Rat | Human | |
| 2901 | cg27216483 | CGGCCCTTATGGCGGGCTGTCAAACTGTTTTGGCTTCCCCGTTATTACTT | | | | Rat | Human | |
| 2902 | cg27236661 | CGCGGGCAGGCAGAGAAGCACCACACAAGAAATTGATTCCATTAAAAAGT | | | | Rat | Human | |
| 2903 | cg27248455 | GGGAAATATATATTTATATAGATACTCTAATATAGACCACATCTCACCCG | | | | Rat | Human | |
| 2904 | cg27251107 | TTACCTGGGAAACCTCTGCTTCAGCTTCCTCAGGCTCTGCCTGGTAGGCG | | | | Rat | Human | |
| 2905 | cg27295000 | CGGGCCCAAATCTTACAGGCTTGTGTTGGCTTTGACCAATGAAGTAGAAC | | | | Rat | Human | |
| 2906 | cg27484199 | CGCTTCAGGGATTGACTAAAACCTGTTCATGTATAGCAGCAATTTGAGAT | | | | Rat | Human | |
| 2907 | cg27495216 | TGGCATTGAGCTGGGCACTCATGACTCGCTCATGATGTGAATGGCTATCG | | | | Rat | Human | |
| 2908 | cg27531392 | CGGCTCAGCTAAATGCGTGCTGAGTGCTTCAGCCTTAAATAGGGAGAAAA | | | | Rat | Human | |
| 2909 | cg27538829 | CGCCTTCGAGATTGGGCTTTATGCCCTTGGCCTGCACAACTTTGTTTCTC | | | | Rat | Human | |
| 2910 | cg27587645 | CGCTGCGAGCTGAGCCCCTTGCCCACCAGGAAGTGCACGTCGCACAGCAC | | | | Rat | Human | |
| 2911 | cg27625359 | CGCATTATAAAAATATGCACCAGTTGCCAACATGTAATGAAATATTAAGA | | | | Rat | Human | |
| 2912 | cg08074329 | CGAGTGTCCGGCTTTTTGAATGAGCCACGTGTTCAAACTACAAATCAACG | | | | Rat | | Universal |
| 2913 | cg24055081 | AAACATGAATGAATGTGGAGGGCGGAGCCTGGGAAAACCCCTCTGCAACG | | | | Rat | | Universal |
| 2914 | cg24446586 | CGTGCTTTCGGTGAGAACACCGAGTGACGATCTGTTGCTTCCCCTGAGGT | | | | Rat | | |
| 2915 | cg16949290 | CGGTCTCTCTATGGCTGGGGCTGCCTCTGTACAATGGGATAAAATTCAAG | | | | Rat | | |
| 2916 | cg12220493 | CGCTGGATATTAGTAGATGAGGATCATTCTGCTTCAATTGGGCGCCTCTC | | | | Rat | | |
| 2917 | cg23009891 | AGAGGTTAGTTCTATTAAGAGTTCATCAATCACCCGGTGTGCACTTTTCG | | | | Rat | | |
| 2918 | cg06603975 | GCAGCTGAAGGCGCCCCATTAGCATAACACTGATGTTTAATTTTCATACG | | | | Rat | | |
| 2919 | cg10244777 | CGGCAAAAGTGTGGAGAAGATTTAAGCAAATGACTGTGTCAGCTTCTATT | | | | Rat | | |
| 2920 | cg01994205 | CGCCACGATGATGTCCATGAACAGCAAGCAGCCTCACTTTGCCATGCATC | | | | Rat | | |
| 2921 | cg02854502 | AATCAATTATTTGACACTAAGATACTCTCAAGAAAAACTCATCAGTTACG | | | | Rat | | |
| 2922 | cg19835617 | CGCTTGTTGTCCACAGTCAAGGAGGTGCCCTCGGGCAGCTCGGCCGGCTC | | | | Rat | | |
| 2923 | cg19376000 | CGTCTATTTCATGAGGTAGTTTGCATGTCCTTGAATGGAATCTCGTTACC | | | | Rat | | |
| 2924 | cg27166817 | CGCCAGGAGTGTCTCAAGGAGCAGATGGGTGCTACTTTGTTTATAAAAGA | | | | Rat | | |
| 2925 | cg06947421 | GACTGGCCATTTACGCCACCACTTTAAACAAAGATATTTGGTTATTCCCG | | | | Rat | | |
| 2926 | cg09434548 | AGGGTGGGAAGCACATGGGCCACTTTTATGTAACACTCCTCTGATACACG | | | | Rat | | |
| 2927 | cg19665414 | TTACTGGTGATCGCTGCCAAAACTACGTAATGGCCAGCTTCTACAGTACG | | | | Rat | | |
| 2928 | cg25817964 | TGCAGTGAATTTATGGCGGCCATCCATTAGCTCTTACCAAGGCAACGTCG | | | | Rat | | |
| 2929 | cg00127523 | AGCAGGCCTTCCTGGTCAGCCTCTACAAGTTCATGAAGGAGCGACACACG | | | | Rat | | |
| 2930 | cg14590156 | TCACGGGATTATATTGTTCGTTGAGTAGCAGCTTTCTGTCCCAGCTGTCG | | | | Rat | | |
| 2931 | cg20359340 | CGCCAGAAAGAGTCACTAATTATGATATGTATTTGCATATGTTTAATCAC | | | | Rat | | |
| 2932 | cg12274258 | CGTGATAAAACATTGATTGTTGCAGTTTGGTGACCCATGAAATTTGGGGG | | | | Rat | | |
| 2933 | cg22188736 | TCTACTCAATGGATTGTGGTCAAGGCCATTGTAACGTGAGCAGTGCCTCG | | | | Rat | | |
| 2934 | cg02005942 | CGCATTGTTTATCCCTATGAAATGTACCAGTCTGGAGCCAACCTTGTGGT | | | | Rat | | |
| 2935 | cg00462525 | GCAGGCGGGGCTGGTACTTGTGCAAGGACTGTAAAACCACCATCTGTCCG | | | | Rat | | |
| 2936 | cg00012521 | AATTTAAATTGCTTTTGTTCATTTTCCATACTACTCCCCACACTTCATCG | | | | Rat | | |
| 2937 | cg00110098 | CGCCTCTCTGGTGATCTGCACTTAATAGGAATATTTTAAACTATGTAGCT | | | | Rat | | |
| 2938 | cg00199273 | CGCCTAAATCATTGTTACACACTAATTAGTGCTGTACTCTGATTTGAGGC | | | | Rat | | |
| 2939 | cg00557947 | CGGATTCTATTTGCATCCGGAATCGCATGTACATGAAAAATGTGTCCGCG | | | | Rat | | |
| 2940 | cg00741609 | GGCTTCTTCTTCTTGGGGGGCGTCCGGTTCTGATAGGGGTGACCTATACG | | | | Rat | | |
| 2941 | cg01178121 | TTCCACCACAATTGTAGCTCCCTCTCCGGCTTTGAAGTGCCGCGGAGGCG | | | | Rat | | |
| 2942 | cg01198746 | TGAATAAAAGGAATTAACCGTTTCCCTCCTCTGACCTCTGGGAATTTCCG | | | | Rat | | |
| 2943 | cg01314094 | CGGCGGCTCGTGCTGGCATCTACGGGGATACGCATCACAACAAGCCGATT | | | | Rat | | |
| 2944 | cg01397139 | CGGGGGGATTGCTAATCGTATTCAGCATGTTTTGCACAAGAAATGTCAGC | | | | Rat | | |
| 2945 | cg01463338 | CGCTTCATGTCAACTACAATGGCACTGAATGAACAGGTTTCAGCTTTATA | | | | Rat | | |
| 2946 | cg01579023 | TCCCATCTCGGGCACTAAATGATCCCCACCATTTGCAGTAAATATATACG | | | | Rat | | |
| 2947 | cg01696865 | CGGAAAGTGTTTTGGTTTTCCTTGGACCTCGAAGAAGCTATAGAGTTCAA | | | | Rat | | |
| 2948 | cg01908395 | CGCCCTAATGGGACAAGCTCGCTCCCAGAGGAGTGACAATTCCTCCTAAT | | | | Rat | | |
| 2949 | cg03045079 | CGTCCTCTACCTCCACCAGGACCAGAAAGGGCCAGCAATCTGGCTTGGAT | | | | Rat | | |
| 2950 | cg03126469 | CGCAGAGACGACACTGCATGTTAGGGGGACCCCAAGAATACCACTGGTAA | | | | Rat | | |
| 2951 | cg03707960 | ACCAGGAGGTATTTACTGCTTCCTGGACAAAGGTCAGGTCCAAAAACACG | | | | Rat | | |
| 2952 | cg04986697 | CGAGCTTGTAAATACAAGCTCCGTGGACAAAGTCCCAAATGACATCACTG | | | | Rat | | |
| 2953 | cg05201058 | CGGAGAGCTGCAAGCCGGTGCAGCAGCCTTCAGCTTTTGGCAGCATGAAA | | | | Rat | | |
| 2954 | cg05907696 | CGTGTGAGAAAAGCCTGGGAACAAACGGGGCGCCTCCGTCTCCAAGAGCT | | | | Rat | | |
| 2955 | cg06053159 | CGGAGTGATTGTAAGTAACGCTTGCCTTAAGAATCTCATTATTGGAACAC | | | | Rat | | |
| 2956 | cg06071218 | AGCTGTGGGTGCTGATCTGGCTGTACTCGCAGTCTTTCATGGGCCCGCCG | | | | Rat | | |
| 2957 | cg06138703 | CGGGAAGAGGACTCCTTTGACAGTGATAGCACAGCCACCTTGCTCAAGTG | | | | Rat | | |
| 2958 | cg06493086 | CGGTCCCAGAAGTACTGCACAGCAATGGCAATGCCCCGGCTGGAGAAGTA | | | | Rat | | |
| 2959 | cg06509306 | CTTGAGTTCTTGGGAGCTGGTGGCCAGCATACTTCGAATGCTTTTGTCCG | | | | Rat | | |
| 2960 | cg07550523 | CGGCGCTGTGGCTCCTCCAGATAAAGCAGCGATTTCAGCCCAAATGTTTC | | | | Rat | | |
| 2961 | cg07665308 | TCAAGACGGCGCCAGGCAGCTCAGTGTTCGCAGACAGCTGTGGCGCGACG | | | | Rat | | |
| 2962 | cg07670450 | ATTTCACTTGGGGAACTTCAACTCTGAGCCACAGGACAGAATAAAACTCG | | | | Rat | | |
| 2963 | cg07887668 | TCTTCCTTTATTGTGCAGTAAATTTCCCTCCAGAGTTCGGATTTCATGCG | | | | Rat | | |
| 2964 | cg07919443 | CGGCATATTTGCTGCTTGGCTAGCCAAATAGAAGTTGTTTTTTGCACAAA | | | | Rat | | |
| 2965 | cg07963404 | CGAGAACAAGCCCATGATCCTGCTGGTGGGCCAGTACAGCACCGGCAAGA | | | | Rat | | |
| 2966 | cg08373745 | GTCCAGAGAAGTAAACTCTCTCCTGTCTTGGACGGGGTCTCTGAGCTTCG | | | | Rat | | |
| 2967 | cg08657007 | CGCAAGGGCAGGGACCCCCCACGAGATTCATGCAGCATTCAAAGTGAGTG | | | | Rat | | |
| 2968 | cg09311277 | CATTCAATTTCCTTAATTTATCCTTGAGTTTATTGCTGCTTTTGAAGTCG | | | | Rat | | |
| 2969 | cg09627707 | TGAGATTCCATTTTCACATTGGTTTGTGCTGAGCATATAAATTTCCTGCG | | | | Rat | | |
| 2970 | cg10302985 | CTGACCATTTAGATTTCAGAGGGATCCAAAACCAATTTAGACTCCAGTCG | | | | Rat | | |
| 2971 | cg10759434 | TAGCAGCACACACGGCATTTTCTCATTCTCACCTGCCAATGTCATCTCCG | | | | Rat | | |
| 2972 | cg11304240 | GCTTCCAGTTGGTGCAGGCCACCTTGGGCACGTAGCAGTAGATGAGCTCG | | | | Rat | | |
| 2973 | cg11448454 | ATGGGCCAGGTCTCCAGCTTCCTCGCTCCCATCCAATGATTTATTACTCG | | | | Rat | | |
| 2974 | cg12010100 | TGGAAGCGGTGAGATATGTAATGCTTTATTAAGCGGTCTCAGCTACAACG | | | | Rat | | |
| 2975 | cg12101354 | ATTCATTTCCTTGGGAGCACTGCAATCCTGCAAACACAATGTCAAGTTCG | | | | Rat | | |
| 2976 | cg12762301 | GTTTGCCATATCAGTTGCATTTGTCTTTGGTACAAAAAATAAAACAATCG | | | | Rat | | |
| 2977 | cg12827555 | ATCACCGGGGCCAGGAGCGATTAGGAATTCCAAAAAGCTGGCAGCTCCCG | | | | Rat | | |
| 2978 | cg12952128 | AGCTTGGCACGCAGACACCGGACGAGAGAGACTGCAGCAAGGGTTTCACG | | | | Rat | | |
| 2979 | cg13076512 | CTCTTTTTTCTCCTTTTCCCCCTGCCTGGAAAAATGGCTAGGTATCTACG | | | | Rat | | |
| 2980 | cg13165254 | GAGGTAACTGAGGACTCTGATGAAGACTTTATACAGCCCCGCAGAAAACG | | | | Rat | | |
| 2981 | cg14161220 | CGGCTGGGTTTGTATTTGCTGTTTTAATACAAACCCCGCCGCACTGCCTA | | | | Rat | | |
| 2982 | cg14305829 | CGGCCTTATTCTATTAGATAAGATGTGTTAATTACCCTGATGGCAATATG | | | | Rat | | |
| 2983 | cg14877464 | CGGAAGAGCGCTTCTCCAAATTGAAATATAGCAGAGTTTACACTCCTGAT | | | | Rat | | |
| 2984 | cg15085822 | ACATAATAATGACATATGCAGGAAATGCACCATTATTTATACATGTGACG | | | | Rat | | |
| 2985 | cg15178575 | TATGGTGTTTTGGTTGGCATGGTGACCCATTCTTCCTCATTAAACTAACG | | | | Rat | | |
| 2986 | cg15479466 | CGGGTAGAGTGTAAATTACATGTCGCCAGCATAAAATATTAAATCCAATT | | | | Rat | | |
| 2987 | cg15772525 | CGCGGGTATTTTAAAGGAAATGAGATCCGTGTGTTTCTGATTTATTTATA | | | | Rat | | |
| 2988 | cg15843929 | CCTAGTTATTTACACTCTTTTTCAAGCTTCGAAGCTGTTATAATCATTCG | | | | Rat | | |
| 2989 | cg16049333 | CGGGTACCCCAGGGAGTACAAACACCTTTTATGACTCACCAGGAACATTC | | | | Rat | | |
| 2990 | cg16117415 | CGGGGTCCTTAATGACCCTGGCAGGCCAATGATTTTGTTTTGTTTGAGAG | | | | Rat | | |
| 2991 | cg16264705 | CGGGCAAAATTACATTCCTGTAATGGCGTCGCTCGGGGTCCCGGGAAATT | | | | Rat | | |
| 2992 | cg16895007 | CGGGCTCTCGCTCCGCCGCTCCATGCTGCTCCGACTCGGAGACAAAGTTC | | | | Rat | | |
| 2993 | cg17174012 | CGGTGGAATCTGTTTTTCTGCCGCCATATGGTGTCGACAGAATGCCACTT | | | | Rat | | |
| 2994 | cg17399859 | CGGCCTCTACTGGTGTGTGTTTATAGAAAATGTTTCAATAGGCAGAAAAA | | | | Rat | | |
| 2995 | cg17514125 | CTGTAAGTGGTTCAACGTGCGCATGGGGTTCGGCTTCCTGTCCATGACCG | | | | Rat | | |
| 2996 | cg17835092 | CGCCAGCACAACAAGGACAAGCCCTACAAGTGTCCCAACTGCTACCGGGC | | | | Rat | | |
| 2997 | cg17952647 | CGGTTCTTTGGAGGCTCAGTTTTTGGTAATTAAGACTAAGGAGTGTTTAA | | | | Rat | | |
| 2998 | cg18184692 | CGCGGTAACCTTTGCAGATGGCGCCGCTATTGCTGAGAAATGGATTGCTT | | | | Rat | | |
| 2999 | cg18335591 | CTCAATCGGCTTGTTGTGATGCGTATCCCCGTAGATGCCAGCACGAGCCG | | | | Rat | | |
| 3000 | cg18479022 | CAAAGCAGCCTCATGCAAAGCACTGCCCATCTCCGTCTGGAAAGCAAACG | | | | Rat | | |
| 3001 | cg18506907 | CCATTACTGTCTCAAGAGCTATTCTGATTAAGGCTAACATGATGTGATCG | | | | Rat | | |
| 3002 | cg18576402 | CGGCCCCAACGATGGCTTCCTGGCCCAGCTCTGCCAGCTCAATGACAGAC | | | | Rat | | |
| 3003 | cg18594436 | AAATCCTTTCAGCTCTTCGCCAAACCCCTAGTGAAAGACAATCAGGCTCG | | | | Rat | | |
| 3004 | cg18800235 | CGGGCCAGCCTGCCTAAGGACAGGAGCCCCGAGGAGTATGGAATCACCGT | | | | Rat | | |
| 3005 | cg19148866 | GGGCTGACAGTGATTGGCAGTGGTTGCCGTGGCAACGCCACAACGACACG | | | | Rat | | |
| 3006 | cg19176697 | CGGATGTCACAGCTTTTCCATAGAACAATTGGAAGTGGAGCTAGTAAGTA | | | | Rat | | |
| 3007 | cg19424122 | CGGTGCCTGTGTCTGGGCTGTGTGAACCCCTTCACCATGCAGGAGGACCG | | | | Rat | | |
| 3008 | cg19426872 | GAAAACCCAGGGCACTGCGACTTTGTGAAGCTGAGGACAATGCTGGTACG | | | | Rat | | |
| 3009 | cg19499679 | TTGAAAAGCTTTGTGTTAACAGCCTTCAACCAGGCTCCAAGAAGAGTACG | | | | Rat | | |
| 3010 | cg20041510 | CGTGTCCAGGACAAAATCTTCCGGGCACATAAGGTGGTACTAGCAGCTTG | | | | Rat | | |
| 3011 | cg20095345 | TCCAAGAAATGCAGCTAAAATTGGCCCTCAAACAGCTTTTGCAGAGCACG | | | | Rat | | |
| 3012 | cg20116240 | TATTAAACTACATGCTTTGTCTTGTGCAGGTGTTGGCTTATGGAAAGACG | | | | Rat | | |
| 3013 | cg20153801 | CGGCAGGACTCGCCCATTGTGCCACCCAGATAATTATTCAACTATGCAGC | | | | Rat | | |
| 3014 | cg20870434 | GCTAGTTCTGGCCACATACTGTCAAATTTTCCAGAAGAAGCATGCTGCCG | | | | Rat | | |
| 3015 | cg20889526 | CGCAGCTGGCATTTACACACTCAGCATTACCATATGTATAGCTGCTCGGT | | | | Rat | | |
| 3016 | cg21388654 | CGCCAGTTTTACTACCTGGGCCTTCCCCAGACGTGTGCCTGTTAGTGGTG | | | | Rat | | |
| 3017 | cg21532698 | GCAGGTGATGTCATGCCCATTGTTTTGGTGCGCCCAACCAATCGGACTCG | | | | Rat | | |
| 3018 | cg21789423 | CGGCACCAGTAGTCAAAGGCCCCTCGCCACCCATCAAAAGTGACAAGCAC | | | | Rat | | |
| 3019 | cg22679201 | AGCTGACATTATGCTGTAGGATGTGCTGCAGGTGTGCAGGCCTGGGAACG | | | | Rat | | |
| 3020 | cg22998308 | GGATGCTCATTCCTTCTACAATTTCATCTACTTCATCCTCCTCATCATCG | | | | Rat | | |
| 3021 | cg23000776 | CGGAGGTTAAATGAACACGGTTGTTTATGAAGACACAGCCCGAATCCATT | | | | Rat | | |
| 3022 | cg23100467 | CGGCAGGAGGACAGTCGGGTGATGGTGTATTCTGCCCTGCGCATCCCACC | | | | Rat | | |
| 3023 | cg23345501 | ACGCCTATAATTTGTGTGAGTTGATCATACTTTGGTAAATCAGCAGGTCG | | | | Rat | | |
| 3024 | cg24066590 | CGAGAGACAAGAGAATAATTCTACGAGGCCGTGTAGAACAAGCAGAGAGA | | | | Rat | | |
| 3025 | cg24314463 | AGTTTGGCTTGTGCTTCTGCAATTTTTCGGTTATTCTCTTCCAGTATTCG | | | | Rat | | |
| 3026 | cg24380618 | CGCAGACCAAACTGGAGCATGCCCGCATTAAGGAGCTTGAACAGAGCCTG | | | | Rat | | |
| 3027 | cg24420398 | GACTCCGGGGAGTATTTCTGCCTGGCCTCCAACAAGATGGGCAGCATCCG | | | | Rat | | |
| 3028 | cg24558834 | TGACTAACTACCTCGCTCAATAGTAATTCATTTAGAGAATATTAAAATCG | | | | Rat | | |
| 3029 | cg24601458 | TGGCTTTAATTTTCCAAATCTGCAAATGCATCCGTTATGTCCCCGCATCG | | | | Rat | | |
| 3030 | cg24604427 | ACAGTCAGCGGAGAGCAAGTTTGCTTGTTTTCTTCCCTGTTAACAAATCG | | | | Rat | | |
| 3031 | cg24780501 | TGTGATGCGTGGGCTGTCGGGACATCTGTCTTCTATACCTAATAAAGTCG | | | | Rat | | |
| 3032 | cg24799293 | CGCGAAGATGGCTGCTGGCTGCCTGCTGGCCTTGACTCTGACACTTTTCC | | | | Rat | | |
| 3033 | cg24884825 | TCTTTTGGAAATTCTCTATTCTCTCACCTTATTAATGTTAAAACAAAACG | | | | Rat | | |
| 3034 | cg25420162 | CGAGCCACATGCGGTAGTGTGTAGCAATTAGTAGATAAAATGCTGACTAA | | | | Rat | | |
| 3035 | cg25473942 | GGTGCCATTTAGTGGCGGGCTCAATGGCGTACTCTCTGAAGGTCACTTCG | | | | Rat | | |
| 3036 | cg25560850 | CCATCTCCCACGGATTCTCCTAGATCCCATCAGATTGTCCCTGGAGGTCG | | | | Rat | | |
| 3037 | cg25799882 | TTCTAAATCAAATTCAAGGGTCACTGATTGTTAAGGCAACCATGGGTACG | | | | Rat | | |
| 3038 | cg25934764 | CGTCGCGCTGACATTGAAGTGTAATTTTTCATGGTTATTTGGACTGGAAA | | | | Rat | | |
| 3039 | cg26058975 | ATGGTGGGGCTGGACGGCACTACTGCAACAAAGCAAACACGGTCAGCACG | | | | Rat | | |
| 3040 | cg26077613 | CTAACTCTCCCTACGGCAGTAAGTGTCTGTTTTTGTCACACGTCATCACG | | | | Rat | | |
| 3041 | cg26352942 | CGTGCCGGCTTTCCTCAGCAAGCTGTGGACGCTTGTGGAGGAAACCCACA | | | | Rat | | |
| 3042 | cg27093665 | ACGTTCTTGTCATTCCTTATGTTACAGAACATCAATCATAAATAAAGACG | | | | Rat | | |
| 3043 | cg27344462 | CGGTAGCCGTCCTTCAGCAGCTGGATGTTCAAATCTTGGTTGATCTGCTG | | | | Rat | | |
| 3044 | cg09817427 | TAATTGAAATTTGTTGCCTTGATCTGTCAGCACTTCCAGGCAGGAGAGCG | | | | | | Universal |
| 3045 | cg22188876 | TGGGCATGGCACACACTGCTCCGTCTGGCCGTGGTAATACGTTCCCTGCG | | | | | | Universal |
| 3046 | cg23627331 | CGGATACATCTAGGGTTGGCATTTCAAGGAATATGTGTGGGTTTTTCGTC | | | | | | Universal |
| 3047 | cg07875786 | GTAGTGTCAAAGTTCACTATATACAGACATTCGCGCAGATCCCCCTTTCG | | | | | | Universal |
| 3048 | cg12492738 | CGTGTGTTATTTTATTTGTCTAGAGAGAGCCTATTTGTTCAGTGATGTGT | | | | | | Universal |
| 3049 | cg19604874 | CGTAGAGTGACGTTTTCTGTTGTGAGTCAGCCTCAGGACCCACATCAGGG | | | | | | Universal |
| 3050 | cg22380921 | CCGGGGGGGGGATTACCTTGTAGCAGGAGCCCGCAGACACTGTAGAAGCG | | | | | | Universal |
| 3051 | cg12933950 | CGCCTTTGATGAAATCCCAGCAGGCTTAAACAATAAAGCTGGTTACAAAC | | | | | | Universal |
| 3052 | cg26973693 | CTCTGAACCTTTAAAGTGATGTTGCTCCAATTACAGAGAGACATTGAGCG | | | | | | Universal |
| 3053 | cg11948733 | CATCCAAGTAGGCCTGAAACGTCTTGGGCTTTGAAATCCGCACCATGGCG | | | | | | Universal |
| 3054 | cg01090334 | CGAGAGGACCCGAGTGGTGTTATCTTAACTTTGAAAAAGCGACCTCAGAG | | | | | | Universal |
| 3055 | cg19981759 | AATTCATTTATCTGCAGGAATGATTGCTGCTATCAGTCTCGCGCTCACCG | | | | | | Universal |
| 3056 | cg13153497 | GGCCCTCTCTCCGGCTCACCTAATGACCAGGAGTTCGTGGAGCAGATACG | | | | | | Universal |
| 3057 | cg10930156 | CCCCCCTTGATTGCTGATTTTACGCTTTTTGGACCAATTGTGGGTCTCCG | | | | | | Universal |
| 3058 | cg12733826 | CCCTTCCAATTGGATCACGACCCTCTGTGTGCTCAGCTTAAGACCAGACG | | | | | | Universal |
| 3059 | cg05191959 | TGCCATGGGGACAAGGTGGGAGCCGGGGATGGCGCGCGCCTGGGCTCGCG | | | | | | Universal |
| 3060 | cg08938156 | TGTCTTTTGCGGTTTATCTTCCTGGGGAGAAACTTTCACCTCCTCAGCCG | | | | | | Universal |
| 3061 | cg20370622 | CGCTTCTAACGTGATAAATTCTTTCATTTGTGTAAGCAAATTTCGTTTGG | | | | | | Universal |
| 3062 | cg13702222 | CGGTTCCAAGCAGCAGAGACGTCAGGAAAGGCACTTCTTAGTACCAACCT | | | | | | Universal |
| 3063 | cg20635820 | CGGGACCAGCTGCTGGCCACATTTCCTGCTTTATTTTAAAAGGTAGTATA | | | | | | Universal |
| 3064 | cg00096922 | CGCGCAATTAAGGATTTAACCAAGGCTGTGCTGCGCCTCAGCCAGCAGTC | | | | | | Universal |
| 3065 | cg01352789 | CGCCAGTTCCAGGACTGGTGCCTCAGGACTTACGGGGACTCAGGCAAGAC | | | | | | Universal |
| 3066 | cg03909491 | GACAGGGTTCAAAGTTTTAATAATATTTGGATAATATCATTGCCCACACG | | | | | | Universal |
| 3067 | cg11742933 | CGCTGTGAGGCACCTGCCAAGAACATCGCCACCAGCCTGCATGAGATCTG | | | | | | Universal |
| 3068 | cg00114412 | CGGCCATTCTGACTCTGCGCCAGCAGCACTAGGCAATTGTGTTTTTAGGA | | | | | | Universal |
| 3069 | cg12564910 | GCGCCTCAAGACCTTCAGCTCCAAGAGCGAGTACCAGCTGGTGGTGAACG | | | | | | Universal |
| 3070 | cg16159306 | CGGTCCAGCTGAGCATCCCTTCCACTAGAGGGCCATGTGACACCTAAGGA | | | | | | Universal |
| 3071 | cg07172031 | CGAGACTTTTTACTTGGATTAATTACATGACACTCAGCAAAGAGCTTCTC | | | | | | Universal |
| 3072 | cg15571405 | CGCAAATACAGAAATCAAATTAGGAAAACAAGATATGTGGGTGTGAGGCA | | | | | | Universal |
| 3073 | cg24707133 | TACAGGTCATACCTGGTGGCATAGGCTGGCTTCACCTCGTGGGGGTTCCG | | | | | | Universal |
| 3074 | cg11488183 | CGAGGAGGAAAAGGACATCTGATTCATAGCAAGCCCTGCCAAAGTACAGC | | | | | | Universal |
| 3075 | cg11610869 | CGAGTAACCTCTTGTCATTTTGCCCTCCAGGACCTGAGCTAATTGGTGTA | | | | | | Universal |
| 3076 | cg18433728 | CAAAGTCGTCTGTGACAATCTGGCAACCTCTGAAACTCTTTGCTTATTCG | | | | | | Universal |
| 3077 | cg17829569 | TGACACACTGATTTAAACATTTATTCACTCCAACTACACAAATACACACG | | | | | | Universal |
| 3078 | cg08247363 | CGTGTGCCATGCTTGTTTGCCTACCAGTGCTGCTTAGATTGGCAAATGCC | | | | | | Universal |
| 3079 | cg14880272 | TGGTAGAGATGCCACACTCGCTCCGCGGTTCGCATGGCGCTCTGAAGACG | | | | | | Universal |
| 3080 | cg22903204 | CGGATTATTTGAATTTCATTATATCTATTGATTTGGGAGCATTTCATCTT | | | | | | Universal |
| 3081 | cg21907062 | AATTAAAGTTATCCTGGACAAAAACCTCATCTGTAATGACTAGCTAACCG | | | | | | Universal |
| 3082 | cg03663497 | CGGTTTTGAGTTAAACTGGGTGAAGTAGAAATATTTTACTCATCAGCTTT | | | | | | Universal |
| 3083 | cg20452275 | CGGCTTCCCCAGCTCTGGCACCCTATTCTGGGCACCACGCCCAGGGCTTG | | | | | | Universal |
| 3084 | cg10146459 | ATTCTTACTCCCTCGAGGAGAGCCACAGGTTGCAAATCCAACCAACCTCG | | | | | | Universal |
| 3085 | cg00296110 | GATTTATCTTGTAGTGGAATTAGTATTCCCCCAAAGGGCCCGGCGTGGCG | | | | | | Universal |
| 3086 | cg24653269 | CGGCCCCAGAGCCCCAGCCCGATCCCAAAAATAGCCCCAGCCTGCCCAAG | | | | | | Universal |
| 3087 | cg17718834 | CGGCGGCTTCATTTGCAAATGTGAACAGATTCTCAGAGCAGATAAATGAA | | | | | | Universal |
| 3088 | cg25943666 | GATGCCTAGGGGGTTTATACTGGAAGGACCAGAGCCGCAGCCTCTACCCG | | | | | | Universal |
| 3089 | cg06878766 | TGAATTTATATTAATTTTTCCCTTATTTTTCGGGTCTCTAATGTCTTCCG | | | | | | Universal |
| 3090 | cg14517144 | ACCTTTGTTCATTAGATACACATACCACCATCATATCCATCACCTCCTCG | | | | | | Universal |
| 3091 | cg11677981 | CGTGTTCTGCAGTTTAACTACTTCCAAATTCTTTGTTCAGGTCACCCCGG | | | | | | Universal |
| 3092 | cg19109204 | TGCCAGCCCCCTCGTCTGATTACATCTAGTAATTCTCCACTGAATGAACG | | | | | | Universal |
| 3093 | cg26294431 | CTCATTTGGACCAAAACAAACTCCTCCAAACTGCTACTGACTGCTAACCG | | | | | | Universal |
| 3094 | cg11666770 | CGCGCCGCTTGTCTCTTAATCTCCAGAGCGACCGATCGATTCGCTATTTC | | | | | | Universal |
| 3095 | cg00250826 | CGGCTTAATCTCCTGGGATGAGTACAGAAACGTGACTTATGGCACTTACC | | | | | | Universal |
| 3096 | cg06721379 | CTTTGAGGAGATCCAGTTTTTTAATGGCCACAACTATCACAAAGGCATCG | | | | | | Universal |
| 3097 | cg10893516 | GGAGCGCCTGCGAGCTGGATCTGGTGGAGGATGCTGCGGCAGGTGCTTCG | | | | | | Universal |
| 3098 | cg26014931 | CGCCTGCGGCCACCGCCATGTACCTGCCTACTTAGCCCAAGGGTTAATTA | | | | | | Universal |
| 3099 | cg12722365 | TGAATGCCCAAGCTGAATAGCCATTTGAATGAAGGCTTTAGTTTGATTCG | | | | | | Universal |
| 3100 | cg12156848 | GGCAGGCATGGTACAGAGCGCTTCCTTGCACAGCCTTGCCCATGAGCTCG | | | | | | Universal |
| 3101 | cg08538268 | CGGCAACTCCAAACTAACATGGCAGTCAGACTGTGTGATGTGGCTTCTCT | | | | | | Universal |
| 3102 | cg03127034 | CGGAAATTGGGGTCATTCTCATTTGCAAAGCGGAGGATCGGAGCCCCGTA | | | | | | Universal |
| 3103 | cg01550545 | ATCATTGTTGAAATATTCCCTTTGAACTGCCGTGTCCAGGGGGGTCTCCG | | | | | | Universal |
| 3104 | cg00467357 | TAATTGTTCTCCCTGGGAAAAACAGAATGGCATGCATTTTCCACAATACG | | | | | | Universal |
| 3105 | cg01450059 | CGTGGGAATTTAGCTAAAACTGTTAAAACTGACACCATTGGCTTTTGTTA | | | | | | Universal |
| 3106 | cg15746205 | CGTGGAAACTTTTTAACATGCTTTGTCGCATTTATATGCTACAGGTTACA | | | | | | Universal |
| 3107 | cg18368305 | GAATGGTTCCTTCTCATCCAACTGCCCATGCGCCAATGATGCTAATGACG | | | | | | Universal |
| 3108 | cg06607996 | CGGGGGCTGGGCTAACAATGCACCATCAGGCCCTTTGTGTGCCTGCACTT | | | | | | Universal |
| 3109 | cg01429475 | AACACGATTTAAAGGCACGATGACACTGATTACTGACCCTCTGCCATACG | | | | | | Universal |
| 3110 | cg12880090 | TTCCAAAGCCAAAAATTTGGACATTGACCCCAAACTGCAAGAATACCTCG | | | | | | Universal |
| 3111 | cg10916192 | CGCCAGCCCGCGGGTGGCTCTCTGCAGGTGTCAATTTATTCCAGAGATGA | | | | | | Universal |
| 3112 | cg09364634 | AGATGACACTAGGACATTGACATAAATGGAGCCATAAAAAGACTCACACG | | | | | | Universal |
| 3113 | cg09169719 | ATAGCACCATCACCTTATTAGGTCACAGCAGAGTGAGTGTCCCAATGTCG | | | | | | Universal |
| 3114 | cg09984378 | ATCTTTTAATAAACAGTTTACTTTTATTTAACTTGTTGTGCAAAATCACG | | | | | | Universal |
| 3115 | cg10439738 | CGGATGAGCTGTCTCAAGGCCATATTCCAGGGGAACGATAAGAGCTGTAA | | | | | | Universal |
| 3116 | cg11235379 | CCAATCACTGGAGGTTTCAAGGAGGCAAATGGGTTCCTTGCGGCAAAGCG | | | | | | Universal |
| 3117 | cg26081366 | TTGCCCCCCTACTGGGGTTATTTGTCCTTGGGTTGAAGGGTTGGAAATCG | | | | | | Universal |
| 3118 | cg23646867 | TACCAATTTTAATTTGCCTCTAATTAAATCCTGTGTATTTTTCCGACTCG | | | | | | Universal |
| 3119 | cg20945212 | CGCTAGTAATTCTCCCGTGTGGTTGAATTCTACCGTAGAGATAATGTCAG | | | | | | Universal |
| 3120 | cg00384342 | AATAGCACAGAAGTCAGAGGCAAGTTTGAAAGTACACCAGAAATAAAACG | | | | | | Universal |
| 3121 | cg02360445 | CGCACTGGTCTCTTAAAGAGTGCCTAATTTATAGTAAATAGCAAGGTCTT | | | | | | Universal |
| 3122 | cg15547854 | CACGTAGCCCAAGCGGCCGATGGTGATGGCTGGAATGGCAGAGGGACTCG | | | | | | Universal |
| 3123 | cg05844902 | CTCATCCTTACCATTTTGACAATCTGGTTATTTAAAAATCATCGATTCCG | | | | | | Universal |
| 3124 | cg16853731 | TTTTTATTTATTCGTGGAGCCTCTCGGCTCCTGGGGTCCTTCTAACTCCG | | | | | | Universal |
| 3125 | cg21351544 | CGAATGGCCCAAGGAAGCCACCAAATTGATTTTCAGGTTTTACATGACCT | | | | | | Universal |
| 3126 | cg23754860 | GAAAGGCCATGGTGCCAGTTGCGCAATTTGGCAAAACGTGGGCTGTTACG | | | | | | Universal |
| 3127 | cg22122198 | CGAGTGGTAATAAGGACAATAGGGGCCCTGGCTAATTTGACATGGACAGC | | | | | | Universal |
| 3128 | cg05537515 | AGGTATGGCTGCTATGGGGCCAGTTTCCTCCTCTTGTTTTTGGCAGGACG | | | | | | Universal |
| 3129 | cg23778391 | CGGGACCCCTGGCATCGGTATTTATGGGAAAAAAATGGGAAATGTGCTGT | | | | | | Universal |
| 3130 | cg01648457 | CGTTATGTACCTTCAGGTGTTAGAGTGTGAGCGTAACCAACACCTGGTCC | | | | | | Universal |
| 3131 | cg20422755 | CGGGGGTGAAATGCAGCGGCCATTCTGACTCTGCGCCAGCAGCACTAGGC | | | | | | Universal |
| 3132 | cg17856858 | CGCAGAGAGCCAGGTTGCCAATGAAAAAGTACATGCGGTTGTGAAATTTA | | | | | | Universal |
| 3133 | cg17267107 | ATAAAGTTCTCCCTGCGCGTCCCTTCTAAACATTTCACTCAGTGGAAACG | | | | | | Universal |
| 3134 | cg12815291 | CGACCCTTTCTGTAAACATTTTACAGCTCTCCGCGCGTTTGAGTGACGAT | | | | | | Universal |
| 3135 | cg11025714 | GCAGCAGCTGGTTGCTGTTACTCAGGACCTGGGCAGAGAGAGCAGCCTCG | | | | | | Universal |
| 3136 | cg23053149 | CGGGCCACTGTTGCTAACCACCATCCTGGTGCAGGTCCTTCACATCTGCT | | | | | | Universal |
| 3137 | cg02172466 | CGCCTGGAAGCAGCTGGCTTCCACCATACCCCAGATGCCTCAGATCCCAG | | | | | | Universal |
| 3138 | cg20076842 | CTCAACATCCATCTCCATCTTCCCATTCACTGGAGGGCAAGACAAAAGCG | | | | | | Universal |
| 3139 | cg15298634 | TTGATGGCATCTTTGCTCCAGGTGACACCTGCATGCTGGCCAGCTGAGCG | | | | | | Universal |
| 3140 | cg18188156 | GACTCAATTGACACCTCAGTGAACCACAAATTAGCGTCTACAGCTGCTCG | | | | | | Universal |
| 3141 | cg21608448 | CGGCATGTAAGAACACTTGGATTTTGAGTAGCACGCCCACATTTACACCC | | | | | | Universal |
| 3142 | cg25140691 | CGGAAGCTTTAATTCTATTGAAATGGCAGCTGCTCAGCTCCGGGGCACAG | | | | | | Universal |
| 3143 | cg11700079 | AAGTCCCTCAAAATGACGGACGCGCAGGTCAAGACCTGGTTCCAAAACCG | | | | | | Universal |
| 3144 | cg13182287 | TTCTTCTATCAACCCTGCTTAGGATGAGAACAAGTTGAGCAGTGCCAACG | | | | | | Universal |
| 3145 | cg05111574 | CGACACAATGGTTAGTAGTTATCTTCCATTTGATCCGTAATCTTTATTGG | | | | | | Universal |
| 3146 | cg15701287 | ACAATAGTCTAATTTACATGACATTCTCCATGAAGCTATTTTCACTGACG | | | | | | Universal |
| 3147 | cg11642756 | TAGACGTGAACTTGTCGATTGGGCAAATTGTTTTTGGTCTCCAACTGCCG | | | | | | Universal |
| 3148 | cg23337318 | CGTGGCAGCCTGCAGCACTGTGTATGGTTTAAATTAAACCTTTTTTTGGA | | | | | | Universal |
| 3149 | cg15296094 | AAGTCCCAGAGTCCTCCAAGGTGAGCCGGGTTATTCTCAGCACACTCACG | | | | | | Universal |
| 3150 | cg15855274 | ACTTGGTAATAGACCTTTTTATGACCTATTTGCGGTCGTCATTGGCTGCG | | | | | | Universal |
| 3151 | cg20565386 | CGGGAATTAGACGGGCGAGTTCCAAATTCTTACTGACTTTGGCAGCTCCT | | | | | | Universal |
| 3152 | cg13057591 | GCCCACCATGTACGCCTTTGTGCGGTTCCTGGAGGACAACGTCTGCTACG | | | | | | Universal |
| 3153 | cg14360110 | TTTATTCCGAGGCAGGAGCCCCGGCGTGATTAGGCCCTTTGTAATTATCG | | | | | | Universal |
| 3154 | cg21086539 | CGCGGGCTCTCCAGAGCCCCAGCTATGTTTCCCATTATGTAACCCTGGTC | | | | | | Universal |
| 3155 | cg10945667 | CGGAAGTACTTTGTTTTTTATGCTAATGAGGGAGTGGGGCTTGTCCGTAT | | | | | | Universal |
| 3156 | cg00020468 | CGCAGTGGTGCAATTGACATGAGCAAGAGGGATTTTGTTGAAGAGAGGCT | | | | | | Universal |
| 3157 | cg00098422 | CAAATCCCAAGCCATTAAAATACGACTAATTTTAAGTTAAACAGAAGTCG | | | | | | Universal |
| 3158 | cg00106940 | CGGCCCCGGCGGAAAACCAAGACGCTCATGAAGAAGGATAAGTACACGCT | | | | | | Universal |
| 3159 | cg00314427 | CCCCAACCTGACGGCTTCTCTCTTTACACACAGTGAAGTTCAAGTCCTCG | | | | | | Universal |
| 3160 | cg00362836 | AAAAGTCAGTTTATACCATCCTTTAAAGTGAATCAAAGAATGCATAAACG | | | | | | Universal |
| 3161 | cg00454305 | CGGGGCAGCTCTGGGGCTTTCTGCCTGAAGACCTCTGGCCCCCGCTGCCC | | | | | | Universal |
| 3162 | cg00568797 | TTTACAAACTGTCAATAAGGCAAGAAAAGCCCTGCCAGGAAACACAAACG | | | | | | Universal |
| 3163 | cg00643485 | CGGTCTGTTCCTCAAAAAGGGCAGTCTTTTCAGTGCAACCACTTTAATGT | | | | | | Universal |
| 3164 | cg00689383 | CGGGTCTTGAAGGCATAGTAGGTACAGCTCATGATGAGGAGTCCATTGTA | | | | | | Universal |
| 3165 | cg00788877 | CGGAAATTGAAGCAATCACTAAAAGGTGTCGGGGAATTAAAATGTCCTGT | | | | | | Universal |
| 3166 | cg00864083 | CGGGAAGTGCCTCTCCAACTCTGGCAATCAACACTGTGACTAACAAAGTA | | | | | | Universal |
| 3167 | cg00871371 | CTAACAGAAAGTGTCGGGCCCCCAGTGGGATTTCTGGGGAAGAACTCTCG | | | | | | Universal |
| 3168 | cg00984060 | CGGTGGGCCCAGGGCTTCCCCTTGGAGTGAGCCTGGAGCCTTCTGTGACG | | | | | | Universal |
| 3169 | cg01137681 | CGGCTTGGAAAATCCCACCGCCAAAATTGAGCCCAGCAGCTGGAGCGGCA | | | | | | Universal |
| 3170 | cg01150494 | CGGAAAGAGGACCCTGCTTTACCCACACAGGTTCCTCTGCAGTACAATGA | | | | | | Universal |
| 3171 | cg01169686 | CGGTGCTTTTGACTGACAAGCATATTGTTATGATTATGACTGGACTTCTT | | | | | | Universal |
| 3172 | cg01224972 | CGTGTCTTTATAAACAGCTTATCTCCTTGAGAACAAAGAATAAACAAGGA | | | | | | Universal |
| 3173 | cg01293647 | CGCATGCCAATCACCGTCTCTCATGTGATAGCTGCTGCCCGTGACGTGCC | | | | | | Universal |
| 3174 | cg01301326 | CGGGCTGTTAACAGGCAGTTGCAATCAGTTTCCGAAAGGTCAGCCAAGGT | | | | | | Universal |
| 3175 | cg01309159 | CGGTCAGTGGAAACAGGACATTTGGATGGAGTATTTATGCATAAGCGAGC | | | | | | Universal |
| 3176 | cg01328716 | GGAAATTTTCCCCAAAGACACAGCCAGTCTTGGTGCAATTAGTGACAACG | | | | | | Universal |
| 3177 | cg01337143 | TTGAGCCGGGCCTTCATGAATGTGGTCCATGTGTCCTCCAGCAGGAATCG | | | | | | Universal |
| 3178 | cg01534197 | CGACACAGTTCAATGCAAAATCAGATATAAATCTAGCTTAAGAGACTCTC | | | | | | Universal |
| 3179 | cg01585174 | CGTGCTTTTATAAGTATTTGAAAAGAAAATATTTTAAAGATTTGAAATAG | | | | | | Universal |
| 3180 | cg01631669 | GAACACTAATGAAGCTGGCAAAACAACTTTTCTTTTTTTATGGCTGAGCG | | | | | | Universal |
| 3181 | cg01648756 | CCTCTGTTTGCCATAAAGATCTCCAGGGCTGTATTTTGCAAAAGATAACG | | | | | | Universal |
| 3182 | cg01782227 | GTTGCTTTAGCGCTTACGCCCCGCCTCTGGTGGCTGCCTAAAACCTGGCG | | | | | | Universal |
| 3183 | cg01946200 | GATGAGGGTCCCAGCGGATCGGAAATGTGAAGGGTCAGCGCTGCCGCCCG | | | | | | Universal |
| 3184 | cg01998606 | TGGCTTGTGTGTCACCATATGCCGCTCGAGCTGGGTGCGGTAGGCAAACG | | | | | | Universal |
| 3185 | cg02040823 | CGTGCTTCAATCAGCTGGCTGTGTAAGAATTCAATCAGCGTTAATCTAAC | | | | | | Universal |
| 3186 | cg02177464 | CGAGTGCCTTCCCAATGGTCAGCCACCCAGTCTTTGGTCTACATTCAGCC | | | | | | Universal |
| 3187 | cg02184281 | CTGTTGAAGATGACCTCTAGCCAGCACGGGCAGCTGCTGATGAACTGGCG | | | | | | Universal |
| 3188 | cg02273345 | CGAATTGTGATCTGATTACCTGAACTCAGTGGAAGTGATTCTCCTACTCA | | | | | | Universal |
| 3189 | cg02284095 | GCAACATCTGGCAAAACCTTTTCAGCAAATTCTTCCTGGCCGTCAGTCCG | | | | | | Universal |
| 3190 | cg02287493 | CGGTGAGCAAATGGTCTCCGAGTCAAGTAGTGGACTGGATGAAAGGTAAT | | | | | | Universal |
| 3191 | cg02551294 | AAGTTATAATTGATATCGGGGCCCATCACCATAATGGGTTCATCATAGCG | | | | | | Universal |
| 3192 | cg02629039 | CGAGACTGGCAGCATCCGGCCTGGAGTGATAGGGGGCTCCAAGCCCAAGG | | | | | | Universal |
| 3193 | cg02864622 | AGAAAACATATAATGTCCTGTTGAACACAGGCCAAATACATTCCCAGACG | | | | | | Universal |
| 3194 | cg02946452 | CGCTTCTGCACTCACAGTGGTACCACCCTGGCAGGTTAACGCAGCGGGAA | | | | | | Universal |
| 3195 | cg02953590 | TGCGAGAACAGGTACCAGCAGGCTCAGGTGGAGCAGCTGGAGCTGGAGCG | | | | | | Universal |
| 3196 | cg02963786 | CTGTGTGCATTTCATTTCTGTCAAGAGTATGTACAGTTAGGGTTCATTCG | | | | | | Universal |
| 3197 | cg02968236 | AAAGAGCTTTTGGTGAGTGCTCATTCCAGGATAATAAACCAAGCAAAACG | | | | | | Universal |
| 3198 | cg03163184 | CGCCAGGTCCCTAGTTAGCAGCTTCCTTCAGCTCATCCAAGAAGCTGACA | | | | | | Universal |
| 3199 | cg03225697 | CGAAGCTGTAAATCAGGATGTTACATATAAATAGTTTCCCTATAAAAACG | | | | | | Universal |
| 3200 | cg03247467 | ACCACAGCTTGAAGGCTCAAAGCTGTGAAAGATAAATTAACCTTCCCTCG | | | | | | Universal |
| 3201 | cg03320248 | CGGCTTAAAATTAGAGTTGTGAAGGCATTGAAAAACACCAGACTTCCCAG | | | | | | Universal |
| 3202 | cg03387624 | CGGGCAGGGTGACGTCACCGTCTCCCTGACACTCCACATTAAAGGGCTGG | | | | | | Universal |
| 3203 | cg03420509 | CGCTTGTCTTCTCAGCATGGATGTTTAATCTTCCTAGGCTTTGTGAATTG | | | | | | Universal |
| 3204 | cg03600207 | CGCCAGGGAAAGCCCTTCAAAGCCAAATACAAACTTGTAAATCACATCCG | | | | | | Universal |
| 3205 | cg03668418 | CGGCTGTTAATGACTTTGATGTTGATGTGGAAAAGTGAATGACTGCATAA | | | | | | Universal |
| 3206 | cg03683773 | ATTGCTTACCCAGTACATATTAAATCTCGGCAAGTATGATCAAAACTACG | | | | | | Universal |
| 3207 | cg03820088 | CTTTCTACTTCTCTTCATTCTTCATTGAGTTTTTCATTTTTCATACTTCG | | | | | | Universal |
| 3208 | cg03905052 | GTGAGACTGTAAGGTCGAATAAGCTGCCTCAAAACCTGCAAAGGGTAACG | | | | | | Universal |
| 3209 | cg03917360 | AGTCTGTTATTGGCTTTATAATTACATAATTAACTTTTTGTCTCTAATCG | | | | | | Universal |
| 3210 | cg04025518 | ATGTTCAGTTCCAAGGAGTCCCAGGGGGGCATGGGCTACATGCCCAAACG | | | | | | Universal |
| 3211 | cg04103301 | CGCCTGCGCCACCAGAACCCAAGTGCAAACCACCATGTTCAGTTCTGCCA | | | | | | Universal |
| 3212 | cg04111974 | CATGTCCCACTCTGCCATCTAAACACAGGCTTTTTGTTTCTTAAGTGACG | | | | | | Universal |
| 3213 | cg04366185 | TTGGGCAGTTCTGGACAAGTCATGGACTCAGTTTCTCTACCTGGGAAGCG | | | | | | Universal |
| 3214 | cg04368876 | AATTATGCAAATCCTGCTTTCTGGTGTCCATTCAGCGGCAATTTCATGCG | | | | | | Universal |
| 3215 | cg04373810 | CGTGAGGATCAAGGGCTCAATTGGTTGTCGTGAGAACAAAAATGGTGACA | | | | | | Universal |
| 3216 | cg04539386 | GGCTCGGGGCATTTGCTGGATGTCCCAGATGAGAGCCTGGTGGTCATCCG | | | | | | Universal |
| 3217 | cg04584281 | CGCCATAGGCCATATTAAGCAAATTGTCCAGCCTGCAAAGAGAAGGAGGG | | | | | | Universal |
| 3218 | cg04706544 | AGAGACCGCCCTTCTCTCTGCCTGGGTGAAAATTAAACTCTAAAGTACCG | | | | | | Universal |
| 3219 | cg04707251 | GGTTTAGTGTAGTCCCAACTGTCATTGTCATTCCTAATAACATTTAGACG | | | | | | Universal |
| 3220 | cg04725401 | CGAGGTATGCTTTTGTTTGAAACTATCTGAAAGACAGAAAGAAAAGTCAT | | | | | | Universal |
| 3221 | cg04787747 | TGGCTTTAGCATGCTTCTTATTAGGGCTGGCAAAGCTGGGCTGGTAAGCG | | | | | | Universal |
| 3222 | cg04817453 | CGGAAATGACGTCAGCAAAGTGAAGGACCTGTTTACTGCAGATCAAAACA | | | | | | Universal |
| 3223 | cg04847707 | CGTCGGACCATTCAGTTGTATGAAATGTACTTCAAAGATCCCCAGGTAAA | | | | | | Universal |
| 3224 | cg04889095 | TCACTGGCAGTGGACACCACGGAGAGCAGGGACATACGCCGGGTGAGCCG | | | | | | Universal |
| 3225 | cg04913399 | CGTCAGCAATACGCCAAGAGCAAAAAACAGGGTAACTCGTCCAACAGCCG | | | | | | Universal |
| 3226 | cg04996684 | CGCTTCCCAACTGGCAGAAGCATCCCTCAGGGGACAAGTCTTTAAACCTC | | | | | | Universal |
| 3227 | cg05035366 | CGCGTGTGCCACCCCCGCTGCATTGAGCACGGGACCTGTAAAGATGGCAA | | | | | | Universal |
| 3228 | cg05136138 | CGCCATGTCCATGTAAGGGCGGCACGGAGTGATCTAGCTGCATTAGAGCT | | | | | | Universal |
| 3229 | cg05172686 | CGCCTTCATCATCAGTGTCATGCTCATTGCGGCCAACCAGGTGCTGCGCA | | | | | | Universal |
| 3230 | cg05196683 | AGGGTTTTCATTCAGATGAAGATGTGGCCCCCAGTTCCCTGCGCTCTGCG | | | | | | Universal |
| 3231 | cg05243338 | GCTAATGTGCTCTGGTTCATCTCTTTGAGCAGTTCCTTTAAGGCATTGCG | | | | | | Universal |
| 3232 | cg05261703 | CGTCATGCTGCATATAAACATTTCAAGAAAGTCACAATGTATAAGAATGG | | | | | | Universal |
| 3233 | cg05292023 | CGGTGCCTGCAGCTGATTTGATGGGTTGGAAATGCGCACGAAACAAAATA | | | | | | Universal |
| 3234 | cg05330587 | CGGGGAGCCCACCGACTTTATTAACCTGCACAATGCGCGGGCGCTCAAGT | | | | | | Universal |
| 3235 | cg05332360 | CGTGCTTCCTTGCTTTTGACAAGATTTGCTGCAGTCTCATGCGACAAAAA | | | | | | Universal |
| 3236 | cg05359706 | AGTGGTGCCTTTGAATACTTTAACATTCCATTATCCAGGACAAAAAAACG | | | | | | Universal |
| 3237 | cg05401971 | CGGGAGGTCTGGATGTCTGTCTTCCGCTACCTCAGCCGCAGAGAACTTTG | | | | | | Universal |
| 3238 | cg05456580 | CGGCACCAGCATGATTTTGATGAAAATGGAATCATTTACTGGATTGGAAC | | | | | | Universal |
| 3239 | cg05524242 | CGCTTCAGGGCACTGGCAAAATGAAAGCGCTGGTGAATTTTCAGCCCCTG | | | | | | Universal |
| 3240 | cg05551621 | CGCAGATGCCGCTCAGGGTCAGCTTCTTCTGCGGGCTCTGCAGGATGGCC | | | | | | Universal |
| 3241 | cg05584618 | TGAAATGTTTCCACAGTTTATTTTTCTAGGATCTTTTCCATGACTGCTCG | | | | | | Universal |
| 3242 | cg05620664 | CGACCTGGGTCTTGAGAAACTTGAGTTAGTAGATGTTAGTTTTTCTTAGC | | | | | | Universal |
| 3243 | cg05684493 | AGGAGCATACAGCAAACATCAGTTTACAGCATAGCTAGATTTGAAAGACG | | | | | | Universal |
| 3244 | cg05690769 | TGAAGATTTTATAGAAACGACATGAAAACTACATTTACTGACATTCATCG | | | | | | Universal |
| 3245 | cg05704605 | CGGGTCGTGTGGAACTCATCAAGTAAGTTTCAAACGTCCTCCCAAAAGTG | | | | | | Universal |
| 3246 | cg05793328 | CGCTGGGGAACCTTCACACTGTTGGGGAAGATTTTTATAGTCAGTGGGTT | | | | | | Universal |
| 3247 | cg05801021 | CGTGGGCCATTTGTAAGGCCCAATAGACCTATCCAAGTTACTCGCTGTAG | | | | | | Universal |
| 3248 | cg05801832 | CGTGATCGCCAAGTTTCTCTCTGGGAAAGGATCTGGGATAGATTATACCT | | | | | | Universal |
| 3249 | cg05817746 | ACAGAGTGACAGCAATGTGACACCATGGAAATAATTACTAACAAATCACG | | | | | | Universal |
| 3250 | cg05844937 | GGATTGCTGCAAATAAATACACTTTAATTTCAGTCAAAAACTGCTCTACG | | | | | | Universal |
| 3251 | cg05911522 | GGGTTGTAGAAGTTATCAAGTGGGATTTGCGGCGCTCTCTGCAGGAAACG | | | | | | Universal |
| 3252 | cg06004718 | CGCACCAGCATAGAGACCAACATCCGTGTGGCCTTAGAGAAGAGTTTCTT | | | | | | Universal |
| 3253 | cg06102298 | CGCCGCAGCCCAAAGCCCTGCCATTGTGGCAGCTGTTCAGGGCAACCTCC | | | | | | Universal |
| 3254 | cg06333141 | CGTGGTGTAATTGTTGAATCAGCGTATTCTGATATAGTAAAAATGGTAAG | | | | | | Universal |
| 3255 | cg06386724 | GCAGGACCAGGAGCACCAGGTGGAGCTGGAGCTGCTGCGGGACGACAACG | | | | | | Universal |
| 3256 | cg06391468 | TTCTCCTGCTCGTCGGTCATCTTCATGAAGGGGTCCAGGAGATTCATACG | | | | | | Universal |
| 3257 | cg06472065 | CGCTTAAAAGGTTTCCATGGGAACAGTGAGGGGAGATGAAGGCAGAGAAC | | | | | | Universal |
| 3258 | cg06482098 | GACACGACGATGTTGATGGGCGAGCTGGTGCTGTAGGACTTGGCCAGGCG | | | | | | Universal |
| 3259 | cg06679868 | CGATGCCATATACGTTGTAACCTTCCTTATAAGTTGCAAAATTCTGTGAA | | | | | | Universal |
| 3260 | cg06721611 | CTTTCTCATTTAGTTAATTACCGAGTGAGGATCATCATTTCCCCGGATCG | | | | | | Universal |
| 3261 | cg06736685 | TTCTGGTTCTGAACGTGAAACCCTGTTGGAAACAGGTCTCTTGACCCTCG | | | | | | Universal |
| 3262 | cg06787049 | CTTCTGATTAAATATGAATTACTATGAAATTTTTCTCATCATCTAACACG | | | | | | Universal |
| 3263 | cg06790494 | CGGGCGGATTACCGTACATGAGGTACTTGGACAAAGTTTTACGGGGAAGA | | | | | | Universal |
| 3264 | cg06828322 | CGGGTTAAATTTATTGCCAAGCCCTGGCTGGCTGCAAATTCGAATTGCCA | | | | | | Universal |
| 3265 | cg07014363 | GAAATAGACTTTTCCCTCATCCATCATAAGTCAGCGTGACAGGGCTGTCG | | | | | | Universal |
| 3266 | cg07081182 | ATTAATGTGTCAAGCCAAACTAATGCATTTTTTATGGTGTCCATGCTTCG | | | | | | Universal |
| 3267 | cg07225115 | TTTGTGTTTTCCTTACTAGAAAATAATTAGACCTAATGAATATGCAGACG | | | | | | Universal |
| 3268 | cg07293320 | CGGGCCGCTGGGCTCCTGCAGAGTTTTGATATGAAAGTAATTATTTTCCG | | | | | | Universal |
| 3269 | cg07299823 | CGGACAGGAAGCCTTTGGCCGCCTATTAAATCCCACCCATTTCTCCGGGG | | | | | | Universal |
| 3270 | cg07383477 | CGAGTCTCCATAGGATGTCAACAATAAAGGGGTTAATGAAGCATCCGATC | | | | | | Universal |
| 3271 | cg07423797 | CGTGTGCAGTCCTTCTGGTTTTGGGCAAGGTCAAAGCCGGGCCTGCAGTC | | | | | | Universal |
| 3272 | cg07459759 | CGTCTGTTAAACGCTTTGAAGATTGGAAGCAACAGATAAGTACTGGATGA | | | | | | Universal |
| 3273 | cg07601141 | CGGTGACGGGTATTCTTGGGTGGATAATACGGATTACGTTGTTATTGCTT | | | | | | Universal |
| 3274 | cg07776022 | TAGACATTACCTAACTCTTGATTTGTCACACCGGGATCAATAACATTTCG | | | | | | Universal |
| 3275 | cg07843244 | TTTGTTGATTTATTGTACGTTAGCCACCGAGCCGTGCAAAGAACATTACG | | | | | | Universal |
| 3276 | cg07878283 | CAGATAATTCAGAAACTATCTGTATGAGCTGTTTATCAAATTATTAACCG | | | | | | Universal |
| 3277 | cg07881279 | GCATCTCTTATTTCTTTAAATACATTAATCAGCCTAGTGTAGGCAAAACG | | | | | | Universal |
| 3278 | cg07906193 | GGCCTGATTATGGACTGCCAAGACTTTTTGGAGAAAGCAGTTTCTTGTCG | | | | | | Universal |
| 3279 | cg07932605 | AACAGTTTGATTAAATGAGCCATCATAACAATGATGTCTGCGGGAAATCG | | | | | | Universal |
| 3280 | cg07991826 | TGGTAGATGAGGTGGCCCTCAGCGTCGTCCTCTACACTCTTGGCTCTCCG | | | | | | Universal |
| 3281 | cg08030321 | GTGCGAATTTTCCTACGGCAACCACAACAATTGCAGTTTCCAGATTGTCG | | | | | | Universal |
| 3282 | cg08092317 | CGCTTTGGGCATTTTTGTATTTCAAATTTGCAAGTTGTCAGTTTGAAAAA | | | | | | Universal |
| 3283 | cg08321639 | CGTCAGTTTCAAGACACCAACACTATTAATATCAAAGGAGCCTTCGCGGA | | | | | | Universal |
| 3284 | cg08378313 | CTAGTCTTGGGTTACTCATAAACATTCAGCAAAGCAATTTCACTCAATCG | | | | | | Universal |
| 3285 | cg08438614 | CGGCCAAACATGGAACCATTTTCTGTGGCGAATCGCTGAGTTCTACCTGG | | | | | | Universal |
| 3286 | cg08446005 | CGCCCCCTGGACACCAGCCTGCGCCTCAAGACCTTCAGCTCCAAGAGCGA | | | | | | Universal |
| 3287 | cg08454381 | CGGCCTGGAGTGATAGGGGGCTCCAAGCCCAAGGTGGCCACCCCCAAGGT | | | | | | Universal |
| 3288 | cg08508546 | CGGCAGTTTAACCTCCGTGTCTGTCAGCTGTGACCTTTTACCAACCCCAA | | | | | | Universal |
| 3289 | cg08570512 | CGCCTGCTTGCACCAGGAACAGCTGATAGCCACAATCTCTTTACTGTGGA | | | | | | Universal |
| 3290 | cg08627730 | CGCTTGAAGCATATCTATCTGATTAGTGGAGAGAGTGCTCTTCTCTGTCA | | | | | | Universal |
| 3291 | cg08651129 | GGACAGACGATCATCATGCAAATCAAGGTCATCACTTGTAGCACCATCCG | | | | | | Universal |
| 3292 | cg08676748 | CGATTTTACAATTTAATGCAGCAGCTTCCCGCCCGCCTGGGACAGATGCC | | | | | | Universal |
| 3293 | cg08681110 | CGCCCGCGAGCCCATTCATCTGTGCACTTGGGCGTTGGACCCCGCATCTT | | | | | | Universal |
| 3294 | cg08747377 | GGCTCCCACGGAAAATATGCTCAGTGCAGCCGCGTGCATGAATGAAAACG | | | | | | Universal |
| 3295 | cg08909367 | CGTAATGGCCTGATTTAATCACAAAGGCAGAATTAAATTCAGATGGCAAC | | | | | | Universal |
| 3296 | cg08933909 | AACGAGGGGGCTCTAAAGCCTCGATGCTCTACAGGAAGCAAAATCTGTCG | | | | | | Universal |
| 3297 | cg09008979 | AGTGGTTTGGGGTTTGTTCCTTTTCCCTCCTGTTCCTTCCCTCAGCAGCG | | | | | | Universal |
| 3298 | cg09009312 | CCATTAGCACTTTTGACAGATCCAAAGATAACTCTGAAGCCACCAAAACG | | | | | | Universal |
| 3299 | cg09023627 | CGGGGATTGGATGATACAGTTTCCATAGCATCTCGTAGTAGAGAGGTTTC | | | | | | Universal |
| 3300 | cg09061197 | CGCAAAGCCTGCTTAGATTGTATATGTCGTGGCTGCCAAAACTCCTATAT | | | | | | Universal |
| 3301 | cg09191327 | GCCGTACAGGAAGCTGTGCAGGATGTCGGAGGTGATAACCTCGGCCAGCG | | | | | | Universal |
| 3302 | cg09200783 | CGGTGGGCCGCTTCACCTTTTCACAGCTGTTACCATTGAGTGACAACTAA | | | | | | Universal |
| 3303 | cg09313705 | CGCTGGAGTCCTGGGTTTAATAGAGAGAGTCCCCATACGCTTGTATTTAT | | | | | | Universal |
| 3304 | cg09316860 | CGGGAATAATTTGCTTTATTTTATGACAAATGCAGAAATAAAAAGAAAAA | | | | | | Universal |
| 3305 | cg09337540 | TATTGCGCAGACAGCCCATAAATAATTTGCATTATGTTCAGCTGACAACG | | | | | | Universal |
| 3306 | cg09360996 | ATACTCAACATCCTGCTCAGAACCTTGGGGATTCATGGCACTAAAAAACG | | | | | | Universal |
| 3307 | cg09374953 | GCAATCATTGGTTCTAATCCAAGGATTGACAGAGCTACAACACACGCGCG | | | | | | Universal |
| 3308 | cg09377007 | CGTGCCAAAATAGAATAAAATCAGTTAATAGCATAGATTAAGGGTTACTT | | | | | | Universal |
| 3309 | cg09453226 | TGCACAGCGCACAGTACCGGGAAGTCGTTAATTGCTTGTATGGACCAACG | | | | | | Universal |
| 3310 | cg09482204 | TAACTTCTTCCCAAAGTGCAAGATTCTTAAATCTCCTTCAAAACATTTCG | | | | | | Universal |
| 3311 | cg09606114 | GTCGTGTTCCCTCTTGGCTTCCCATTTCCTTTCCCATGGTGTCTGGGACG | | | | | | Universal |
| 3312 | cg09646392 | TTCACTGCTTTGCCCCAGCATTGAAATATTGATTCATCTTTCAACAAACG | | | | | | Universal |
| 3313 | cg09750164 | GCATCTCCATGGGCGTCTTGGGGTCGAGGCCAAAGCCGGGGCTGTTAACG | | | | | | Universal |
| 3314 | cg09809672 | TTCATCTAGAAGGTTTGACTCTGGCCAGACAACCAGCGAGCATCTTCTCG | | | | | | Universal |
| 3315 | cg09989727 | CGAGCATTTGCAAGATGCAATGTCCTTCTTAAAGGATGTCTGTGAGGTAC | | | | | | Universal |
| 3316 | cg10045881 | GCTTCTTCTGGGATACACATTCTCTAGGTCTTTTATCCACTGAGGTTTCG | | | | | | Universal |
| 3317 | cg10128645 | TGGCTTCTGGTTGCTGTATGTGAACGAATGTTGCCTGATTATTTTAATCG | | | | | | Universal |
| 3318 | cg10186512 | CATGTCTGTGCAGTCAGGAATGATAAGTGAACCACCCGTGAATGTTAACG | | | | | | Universal |
| 3319 | cg10211907 | CGGGCAGCTCCAGGAGGGGACGGACAGGAAGCCTTTGGCCGCCTATTAAA | | | | | | Universal |
| 3320 | cg10235290 | CATTGGATTTTATATCACACAAGGACAATGTGCCATGGGTATTAAAATCG | | | | | | Universal |
| 3321 | cg10246778 | AGCATTGTAAGTAGCTAGGCCTCTCCAAATTAATGCCTTGTTTTGCATCG | | | | | | Universal |
| 3322 | cg10381180 | AATATCTTTCTTTAAAATCCTGACCCAAAACACAACGGGGTGAAAAATCG | | | | | | Universal |
| 3323 | cg10466667 | ATCCCTCTCTTCCTTTATTGCTTCCCGTATTAAGACTATTAATAAATACG | | | | | | Universal |
| 3324 | cg10559644 | CGTGTGCCGGATGCTGCCCATCTTGTTGGAGGCCAGGCAGAAATACTCCC | | | | | | Universal |
| 3325 | cg10609096 | TTTCAGGTTGACGTCACTCCCTGCCTGCTGGCTGTCAGGACTGGGGGACG | | | | | | Universal |
| 3326 | cg10677704 | CGCATTTATATGCTACAGGTTACAAAGTGAGAGCCTTGTATACACTTCAA | | | | | | Universal |
| 3327 | cg10681713 | GCCCCTGAGTGCTTATTCATCAACGTTTTCAAAAGCAAAATCACTCTTCG | | | | | | Universal |
| 3328 | cg10710448 | CGGATGTGGTTAACCAGTTTGTATTTGGCTTTGAAGGGCTTGCCCTCGCG | | | | | | Universal |
| 3329 | cg10718271 | CGCTTGACACCCTGACACTAATTTTATTCAAGCCATTGGGAAATAAATGT | | | | | | Universal |
| 3330 | cg10739250 | CGGGAAAGGTTAATTGGGGCCGCCAGAACGCAATCATTCCTTACCTAACC | | | | | | Universal |
| 3331 | cg10757510 | TGGGAATCCCTAGTGATGAGCACATAAAGCTGCCTCTAACTCAAGTGACG | | | | | | Universal |
| 3332 | cg10792727 | GAGGGCGGAGCCTGGGAAAACCCCTCTGCAACGCTGCACTGAACAAAGCG | | | | | | Universal |
| 3333 | cg10932472 | AAGGCTTCCAACTGTGACACAAGTGCAACCATATTGCTTCCTATTACTCG | | | | | | Universal |
| 3334 | cg11082761 | TTGGGGAGAGCCTGCTACAGTTCTTTGTTTGACTCCGGGACTCAGGGACG | | | | | | Universal |
| 3335 | cg11108049 | CGCCCTGTTTCTTAGCGCCGGATCCTGGGGACCAGACACGGGGAGACACA | | | | | | Universal |
| 3336 | cg11128281 | GTCGCTTCAGGATGTGTCGATCCTAGAGGGTTACGCACAAATCGTCGCCG | | | | | | Universal |
| 3337 | cg11156581 | GTTCTTAAACCAGTTGCCTACTTGTGTGGGAGTGAGGCCGGTGGCCTGCG | | | | | | Universal |
| 3338 | cg11160393 | CGCTACGGTTCGCAGCTAATGAGCATGCAGCTGGATTGCAAATGGCTGGA | | | | | | Universal |
| 3339 | cg11215098 | AATACAATGTGACATCTGGAGAATAAATTGTTCAGTCAGCCAGAAATACG | | | | | | Universal |
| 3340 | cg11217865 | CGGCAAAGGCGTTTTTATAGGGAAACTATTTATATGTAACATCCTGATTT | | | | | | Universal |
| 3341 | cg11251514 | GTGCTTCTAAATTACAATCAAACAGTTACCAATTACACAAATTATTGACG | | | | | | Universal |
| 3342 | cg11275232 | ATTTACTTTGAGGGATCCAACTCTACTAGCAACTCCTTTGCTTTCATCCG | | | | | | Universal |
| 3343 | cg11276577 | CTGGGCTTCTTGCAGCGAATTCCACACTCCTCACAAACATATTTCCCTCG | | | | | | Universal |
| 3344 | cg11327514 | CGTCCTTGTCTGGCAAAATCCACCATGCAATATTTCTGTTAGTAGGTATA | | | | | | Universal |
| 3345 | cg11480265 | CGGTCTGTGCTCCTACGTGAAACCCGAGCAACATTAAGGCTTTCAGGGCT | | | | | | Universal |
| 3346 | cg11498355 | AGAATGCTTTCATTGTAAATTCAGGTTACAAAGAAACTATACCTGGGCCG | | | | | | Universal |
| 3347 | cg11514832 | TGACAGCACTAACTCTCCAGGGCAGAACTTTCCAGTCAAGTGGATCAACG | | | | | | Universal |
| 3348 | cg11516694 | CTATAACTATTAGTAGTCATCGAACTGGTTTGTTTCTGGATGGCCGAACG | | | | | | Universal |
| 3349 | cg11664765 | CAGCTGGACGGACTCGGGTGTGGTTTGCAGCTCGGGGGCAAAGCAGCCG | | | | | | Universal |
| 3350 | cg11765970 | CGTAGACTTTGAAGCCGTTTTTCTTGCAAAGGGTGAAATCGAAGCAGGAC | | | | | | Universal |
| 3351 | cg11857072 | CGGCCGGCCTTCTGCCGCTGCCCCCTTGTACAGAATCGAACTCTGCACTT | | | | | | Universal |
| 3352 | cg11904056 | ACGTGTCAGTTTCAATAGTAGTGTCAAAGTTCACTATATACAGACATTCG | | | | | | Universal |
| 3353 | cg11978813 | TTTTTGGAAATTCCTCCTTTCAACAAGCAGTATACAGAATCCCAGCTTCG | | | | | | Universal |
| 3354 | cg12289045 | ACAAAAACACATACACAAACCGGTCACTGTCAGACTGTCTGTGAGAAGCG | | | | | | Universal |
| 3355 | cg12289788 | CGCCTGAGTGAATCAAAATGAATGTAAATGAGGTTTATTAAGTCAACCCC | | | | | | Universal |
| 3356 | cg12314723 | CCAGGTCTCTGTGGCTGGGCTGCTCCAGCCTGGCGGACAGCATGCCTTCG | | | | | | Universal |
| 3357 | cg12325485 | GGCTACCGTGCGGAGAGCGGGCTGTAAATATTTAAAAAGTCTCTCGCGCG | | | | | | Universal |
| 3358 | cg12382554 | ACAAAATCAAAATATTCATGAAACACAATGATATCTCCCCAAAGCAGCCG | | | | | | Universal |
| 3359 | cg12425327 | TGGCATTTATTCTTTATGTAGGAATATTTGTTCCTCAAAGTCAGACTACG | | | | | | Universal |
| 3360 | cg12428891 | TTAGAAGCCTTACCAGCAGGGAAGCCTGCATCTTTTAAAAACAAAACTCG | | | | | | Universal |
| 3361 | cg12449245 | TCTGGGGGCTCAGCCACCGGGGGATTTAAACAGTACATGTGATAGCCTCG | | | | | | Universal |
| 3362 | cg12459916 | GCACTCCGGCAGGTACGGCTACGGCTACAATGGCATGGATCTCAGCGTCG | | | | | | Universal |
| 3363 | cg12460432 | CGTCTGCAAGGCAGTAAAATGTTATGTATTTGCAAGGCCCATAATCTTTA | | | | | | Universal |
| 3364 | cg12501063 | CGTGGGCGACTCGGTAACGTCTGACAACCTTCAAAGAAAGGAGAAAACAA | | | | | | Universal |
| 3365 | cg12577973 | CGTCCCTCAGAGGGGAAGCCATCCTGTGGCTCTGGCCCCCCCACCACCCC | | | | | | Universal |
| 3366 | cg12642246 | AATTCGATTTTAATTTCATAGCAGAGCAGTTGATTCATGGCTCTTTGTCG | | | | | | Universal |
| 3367 | cg12651099 | TCGGAGAAGATCATCATCAACGTGGGGGGCACGCGACATGAGACCTACCG | | | | | | Universal |
| 3368 | cg12685265 | CGGCCCCAGGCTGATGTTGTGGTAATCTAATCAGCTCGGGTCCTCCACAC | | | | | | Universal |
| 3369 | cg12721805 | CGACCCTGGGAGTATAACCAACGAGGTGATTCAGGAATGAATCTGAAAGA | | | | | | Universal |
| 3370 | cg12761282 | CGCCCACTTCATTAGAATTCTAATTGGGGTGTGTAGGAGAGGATTCTATA | | | | | | Universal |
| 3371 | cg12813831 | CGTACATAAAGAAGGGCTGTTAAACTCTTGTTGAGTTATTTTTGTTTTGC | | | | | | Universal |
| 3372 | cg12818872 | AAGTTAGGCGGGATTATTTGAAAGGTATGTCACAATGCTTGACCTTTACG | | | | | | Universal |
| 3373 | cg12830057 | CGCATGAGTTGCTGGCGTAGCAAGATGCGTGATGTCATACTGGAGGAGCT | | | | | | Universal |
| 3374 | cg12896068 | CGCCTATAAATGAGAAGCGAGCTATTAACATTTGTGTATTTTCATTATGT | | | | | | Universal |
| 3375 | cg12942661 | TCCAGATAAGAGGGCGTACCTTCCGAGGGGTCAAGCCTCCGAGCCCTCCG | | | | | | Universal |
| 3376 | cg12965599 | GTAAGTAAATTTTCTCTGGCGATCAAAAGCCTGGTCGGCAACAAAGACCG | | | | | | Universal |
| 3377 | cg12973326 | CAAGGAGATCTGCATGATCAAGGTAGAGATCCAGGACATCAACGACAACG | | | | | | Universal |
| 3378 | cg13006563 | GAAAACTGTTTATGGGGGCTGTCGTCATCTGTGCACAAGCTATTTACACG | | | | | | Universal |
| 3379 | cg13068315 | AGAAGGTTTCAGGTGGGTGAAACCTCCTATTCCATGCGTAAGGTGCCTCG | | | | | | Universal |
| 3380 | cg13087210 | CGTGGCAATTCTCTGAAGCGTTTACAAGAAGAAACCTTGACAAAAATGTC | | | | | | Universal |
| 3381 | cg13151063 | CCTTCTTCTGTATGTTCATTTACACTTCTGCCTTCATCTAGCAATTTACG | | | | | | Universal |
| 3382 | cg13188467 | CGGTTTTAATTACAGTGAGGGTTTCTCTACAAATCTGTACAAGACAGTGG | | | | | | Universal |
| 3383 | cg13242392 | GAACAAAATGCCGACCGGAAGTGCAGCTGCAGGAATGACTCCCTCCGCCG | | | | | | Universal |
| 3384 | cg13275239 | CGGCGCTGCCTCCTGGCTACAGTTACACCAGCCTGCCATACTATACAGGG | | | | | | Universal |
| 3385 | cg13343102 | CGCCCGTTTGCAGGTCGCGTTTTCCGTAATAACCTTTAATGCGGCTGGCC | | | | | | Universal |
| 3386 | cg13379618 | CTGCCTTTCCCCAGCACCATTAGGAAGTTAAAATCGGTCAGTTTCATCCG | | | | | | Universal |
| 3387 | cg13383038 | GGCTAAGTGGACACTTGATTGAATACCACTAAGACAGCTCACATCCCACG | | | | | | Universal |
| 3388 | cg13400013 | TGTGAATAAAACCCTGGTCAAAAGCTCTGTCACATCGCGCTGGCAAGACG | | | | | | Universal |
| 3389 | cg13532184 | CGCCGCCGTAGTTGCTGCTGCCGCCTGCCGGTCCTCTAGCTTCGACAAAG | | | | | | Universal |
| 3390 | cg13589316 | TTTCACTGAGAAAATTGTTTAGAATTGCCTCATTCCTCAGCAGATTAACG | | | | | | Universal |
| 3391 | cg13608832 | AGAAGATGGTCCGGAAAGCCTGGCTGGCTAACAAAGACAGGCCTTCAGCG | | | | | | Universal |
| 3392 | cg13609316 | CGACCCTGATACAGTGGCATCACCCAAACCTGTTTCATCCACTGTTTCAG | | | | | | Universal |
| 3393 | cg13655781 | AGATGCCGCTCCAGGGCTTCATGTGGAAGACGAACATCCAGATGGCCACG | | | | | | Universal |
| 3394 | cg13802785 | CGCTGGAGAAGACAAGTTAGAAGAAAGGACATGAAGCTTCCAACGGCTAC | | | | | | Universal |
| 3395 | cg13830021 | GTCATCCGTCAGAGCTAGAGAGCAGGACGGTTCAGACACAATCTCTGACG | | | | | | Universal |
| 3396 | cg13851328 | CGCGCCAGGGACAGGATGTAGTTACAGGCGATCCTCAGGATGGCCAGTTT | | | | | | Universal |
| 3397 | cg13884903 | CGCTGATCGATATCTTGGTAAAATATTCATTTTTAAACTAGCGGGCAGTG | | | | | | Universal |
| 3398 | cg13916600 | GATTGGCAGTTCCCTGGCCCGGCGCCAGCCTATTGGGAGGCCTGTTTACG | | | | | | Universal |
| 3399 | cg13935553 | CGTCAGTTTGGTGCATTTGCTGCTGCTGTTGCTACTGTTTGCCTCAAACG | | | | | | Universal |
| 3400 | cg13935834 | GGATCAATGCTTTATTAACAAGAAAATGTCTTACTTCACATTCCTGTGCG | | | | | | Universal |
| 3401 | cg13953571 | CAAGCACTATTTGCAAGCGGGAGGCTCAGAAGCCTCGGCATTTGTCAGCG | | | | | | Universal |
| 3402 | cg14002724 | TGTAATTATTCTCTTAGCTTGCCCTTCATAAATTTATCTGCGTTCTATCG | | | | | | Universal |
| 3403 | cg14045036 | CGGCTCGCTGGCCCGGAATAGCTTCACTTTCTGGCACACGAAATGCTTCT | | | | | | Universal |
| 3404 | cg14136214 | GAACACAGGGGCCTTTTAAGTTGAGCGTGCGCACTGCATGGGAAATAGCG | | | | | | Universal |
| 3405 | cg14172937 | CGCCCGCCCGGCGCACACACACTCGCACACGGACACACGCTCACACCCGC | | | | | | Universal |
| 3406 | cg14297772 | CGCTCAGCTGTGCCCAGAGCGTGAGGCTGGCAGCGCCTTTAATCGCCTTT | | | | | | Universal |
| 3407 | cg14299268 | CGGTAGACCAATTAGTGAGCTTTCTAAATGAAGTAAGCTTTTTCATACAT | | | | | | Universal |
| 3408 | cg14361627 | GCCCCCCGGCTAAGTCATGTTTAACAGCCTCAGAAATTATCTTGTCTCCG | | | | | | Universal |
| 3409 | cg14381350 | CGGCCCCAACCCACGCGGAGGCCACGTGCACAGCTCCACCATCCACACCC | | | | | | Universal |
| 3410 | cg14443903 | CGCATATTTGAGTGGTAGTCCAAATAAATGCAATATAGAAATTTATGTTT | | | | | | Universal |
| 3411 | cg14498322 | CGCAGTCGGGCAGATTGGGTTAAACCAAATATTACAATTGCAAGAACTCA | | | | | | Universal |
| 3412 | cg14548925 | CCAGTGTCGCCATTGCTCTTCCTTCTTGTTGCTTTTTCCCTGTTCCTTCG | | | | | | Universal |
| 3413 | cg14551953 | TTCGCAGAGGCTACAGGTCGAAAATAGAGCACGGTGCCAGCTTCACCTCG | | | | | | Universal |
| 3414 | cg14565725 | ACCGGTCGAGGACGGCAGGAGACCCCCGAGTGCAGAGAAAGCTCAAACCG | | | | | | Universal |
| 3415 | cg14640670 | ACAGAACCTCCACCAACTGACAGTTTCTCTTCTTGACTTGCCACCCATCG | | | | | | Universal |
| 3416 | cg14656600 | GGAAGGATTCTTGAAGCCCAAGATAAACAAATTGTTCCTCTTCACGTGCG | | | | | | Universal |
| 3417 | cg14666740 | TATGCAGCAGCCCAGCGAGTTCCACTGCATGATGAACTGGACGCACATCG | | | | | | Universal |
| 3418 | cg14668921 | GATAAACAATTCCCCAAATAGATTAAAACGAAGTGTAACTTACAAAGGCG | | | | | | Universal |
| 3419 | cg14701342 | TTAACAGAAATCCTATACACTCCACTTTGTCAGCAGTTTTGAATTCATCG | | | | | | Universal |
| 3420 | cg14728312 | CGGAGCTGGATCATGGGTTAGTTGAAGTGATATTTTCCGCTAATGGTAGT | | | | | | Universal |
| 3421 | cg14762358 | CGGCGAATAAGTGGGTCATGTCTAATAAGCTATTTGTGTTCAGGAGGAGA | | | | | | Universal |
| 3422 | cg14794469 | CGGCACGTTTTTGCTTATAAAAAAAGTGCAAAAAAGATGTGGTTTACAAG | | | | | | Universal |
| 3423 | cg14821110 | CGCAGGTGCAATATGAATCAATTATCTTAATTAAGATTATGCCGCAAGCC | | | | | | Universal |
| 3424 | cg14865782 | CCAGGCAGGACGACTTGAGCGCCCGCGCATTGTGCAGGTTAATAAAGTCG | | | | | | Universal |
| 3425 | cg14903956 | GGCACACAGGGGTTGGACTGGTTCCCTATACAGATGTCCTTCTGGAAACG | | | | | | Universal |
| 3426 | cg14919591 | CGTGCCCTGTGTTACTTATTTGGGAGCCGAAAATTTATGCCCAGCTGAAA | | | | | | Universal |
| 3427 | cg14991111 | TTCAGTTTTCCAGGCCAGGCAGCCCGCTATCCTTGGATGGCCTTTCCACG | | | | | | Universal |
| 3428 | cg15006148 | TGAATATGCTAAAATGCATATCTCTGATGCACCTTTAAGCCAGAAAAACG | | | | | | Universal |
| 3429 | cg15009537 | GCTGGATTGCCTTTTGCAATTCTTACTTCAAGGCATACCCCCTTCCAACG | | | | | | Universal |
| 3430 | cg15030317 | CGCCTCAATCTAATTAGGGGTTAAAAGAGGCCATGACTGTGATGTGACAG | | | | | | Universal |
| 3431 | cg15094970 | CGTTTATGGCTTAAAACCCCAAATGGCTGATTTTTTAAAAGAGAGAGAGA | | | | | | Universal |
| 3432 | cg15171944 | TCTAGAGACAAAGTTCTGTAGTGCACTGACTTCTATCATACTCAGAGTCG | | | | | | Universal |
| 3433 | cg15237969 | CGTCCTAACCCAGCAAGGCTAGGTCATTAAAGTGGAAATGAATTCTTTCA | | | | | | Universal |
| 3434 | cg15254020 | CGAAGACCAACTGCTGGAAAATCTCACTGTCAAATCCTCAAGGACATTGT | | | | | | Universal |
| 3435 | cg15391310 | TATCCAGCCTGTGTTTACATAATCTAATTTAACAAAGTCTCAAATAAACG | | | | | | Universal |
| 3436 | cg15405572 | CGGGCTAAAAGTTCTTCCAACAGCTGTTTGTTTTGGCCATTAACATGTCC | | | | | | Universal |
| 3437 | cg15408728 | CGGTGCTAAGATGTTCTGAATTAGATTTGAATTACAACCGCTAAATGCCC | | | | | | Universal |
| 3438 | cg15437942 | TAATTTACTCGTGATCTGTTATTAACCCATCGGGTTTCGAGCTCTTGTCG | | | | | | Universal |
| 3439 | cg15511156 | CCGAGTGCTTGACAGAGATAATCTCAGCAATCCCACGGGTGACCTTATCG | | | | | | Universal |
| 3440 | cg15804249 | CGGAAAACACAGGCCAGTCAAATGTTTCTTATGAACTGTTGACACTATAT | | | | | | Universal |
| 3441 | cg15809618 | CGTTCCGGGTAATCGGTTTTCATAGCACATTCATTCTATTAAAGAGGACT | | | | | | Universal |
| 3442 | cg15813523 | CGAGACCAGCATGAGGTAGAAAAGACCATGCCAATATTTAGCCAGGGAAT | | | | | | Universal |
| 3443 | cg15828158 | CGCTGTCAACAAAATGACAATCAAGATGAATGAATCGGATGCAGTAAGAG | | | | | | Universal |
| 3444 | cg15913680 | CGGCTTGAGGAAGACCTGCTGGCCCCGCCTGAGGAATTGGACAACAGCGA | | | | | | Universal |
| 3445 | cg15991909 | CACAATTAAAGGGGCAACGCACAGATTTCAGGCACCAACCAGCAGCTTCG | | | | | | Universal |
| 3446 | cg16011662 | CGAAGCACAAATCTCATTAGCCACAGGTCAGTAAGGCTTGGAGAGAAAAA | | | | | | Universal |
| 3447 | cg16095149 | CGCGAAGCTCTGGAACAAGGAGAGAGCCAGTATCCACATGCCCCTGCCGC | | | | | | Universal |
| 3448 | cg16100840 | CGCTCCTGACCTTGCAGGACAGCTAATCCATCCGCACATCTATCTTAATT | | | | | | Universal |
| 3449 | cg16118197 | GGGCCTCAGAACCCTATGGTAGCCATGGCAACCAGCCCCTTTAGACACCG | | | | | | Universal |
| 3450 | cg16154416 | GTTTGCCATTTTGTGTTGTCCGAACTCCGCTTGCTCAGGTAGCCTTTGCG | | | | | | Universal |
| 3451 | cg16278638 | GAAAGCGCCCAATAGCTCTTGACACGCAAATGGGTTAACACAATAAAGCG | | | | | | Universal |
| 3452 | cg16378867 | CGGAACCGGTTGAAACCGGTTCAGACTGGGAGATTCCATCTCGGTTGAGT | | | | | | Universal |
| 3453 | cg16454989 | GGAGACTGAGTTTGTGGTGGCTGCTGAGCAGGGTCTGTCTGCTGTTGCCG | | | | | | Universal |
| 3454 | cg16542942 | CGCTTGAAGTTGGGTATCGCCTTTGTGAGCTTTATGAGCCTATTTTTCTG | | | | | | Universal |
| 3455 | cg16550733 | TGGGAGCTTTTCTTTCAGCACCACAACATTGCACCTAGACTTTTTAAACG | | | | | | Universal |
| 3456 | cg16571746 | CGCGTTTGCAGGAGGAGGAGTAAAGCGATGATTGTGTAATTAAATAATAA | | | | | | Universal |
| 3457 | cg16582309 | AACACCTCCCGACCAGGAAGTCATCAGGAATATTGACCAATCAGAATTCG | | | | | | Universal |
| 3458 | cg16591739 | CGCTCTCTCTTGGGCTCCTTGCAGTCCTTGGCAATGTGGCCACCTCTACC | | | | | | Universal |
| 3459 | cg16599143 | AAAAGGCCACCTATTCTTTGCTGTCCTTCACAAGAATGTATCTGAAGTCG | | | | | | Universal |
| 3460 | cg16624401 | CTCGAGGAATACCTCAATACCTCAGACAAGGCCCTTCCAATATGTTTACG | | | | | | Universal |
| 3461 | cg16671155 | CGGCCCCAGGATCACTCCGGCACACTGGGTCCCGAGGAGTTCAAAGCCTG | | | | | | Universal |
| 3462 | cg16717607 | GGGGCCGGGGGGGGGAGCCAGTGCCGAGCTCAGGCTGCACAGTAGCGACG | | | | | | Universal |
| 3463 | cg16745091 | CGGCTGCTCTCACGGCTGGTATCGCGGCTGCACCCCTGACTCATGCTGGG | | | | | | Universal |
| 3464 | cg16764906 | GTACCCAGACAAAACCCGGCCACGTGTAAGGTAATTGGCAGTATAACACG | | | | | | Universal |
| 3465 | cg16848873 | TTATAATGCAGCATTCTTTTGGACACCACACCTAGGTCGGAGCACTGTCG | | | | | | Universal |
| 3466 | cg16886381 | CGAGCCCCCCCAAGGCTCAGAGCGGTACTGGCTTTTAAAGGGAAAGGCCT | | | | | | Universal |
| 3467 | cg17114460 | CGGGGCTGCAACGTCATAATCTCTAGGGAAATCCTGCCAAGAAACGCAAA | | | | | | Universal |
| 3468 | cg17116410 | CAAACCCACAATATATTTACATCATATATAATCTTAACTGTCCAGCCACG | | | | | | Universal |
| 3469 | cg17139066 | CGATACAAGGACATTAAAGAGCATTAAACTACTTGATGACATTTTGGTCA | | | | | | Universal |
| 3470 | cg17213306 | CAATGATTTAATACAACACTGAGGAACAGCTTTCTTTAGTCTTTAACTCG | | | | | | Universal |
| 3471 | cg17251355 | CGTGAGGGTGTTTTAATACCTACTCAGAAATTACTCATGGGTCGGCTTCA | | | | | | Universal |
| 3472 | cg17266282 | CGCAAGCGAGTCATCGCAACAACACTCCCGAGAACATTCTTGTTGGGTAA | | | | | | Universal |
| 3473 | cg17311369 | CGCCTCATCTGGCAGCCGCTTTCTGGTCCAGCAGACACTAACATCTAATG | | | | | | Universal |
| 3474 | cg17318138 | CGGCAGCAGGAAAATTAATTACTGGACGATTTTCACTTCCTCTTGCAGCG | | | | | | Universal |
| 3475 | cg17356452 | ATATGGCATACAGAGCATTTACATACTCACATCTGAAATACTAGGCAACG | | | | | | Universal |
| 3476 | cg17504468 | CGGTAACTGAAGGGGACCAATTGAGCTGAACAGAGTGACTCAGAAATAAA | | | | | | Universal |
| 3477 | cg17516812 | CTGATTGCTGTTCTTTTTACTATCAAGGGACACTCTTGCCCAGACAGACG | | | | | | Universal |
| 3478 | cg17656607 | CGCATTGACCTTTGATCCTCCACCCGGACGGTGTAGGGTTCCCTGAAAAC | | | | | | Universal |
| 3479 | cg17668518 | CGACACGCGAACAATTTTATCAATAGGACCATGTAAAGGCCGACTGTGAG | | | | | | Universal |
| 3480 | cg17769560 | GACTTACGGGGACTCAGGCAAGACCAAGACGGTGACCCGTAAAAAATACG | | | | | | Universal |
| 3481 | cg17783618 | CGAGACATTAATCTTCCAATAGGTCTTTAATTAGGACAGATGCACTAAAT | | | | | | Universal |
| 3482 | cg17818266 | CGCAACTTTGCTGAAAAAGTGTTTGCCTCTGAAGTCAAAGATGAAGGAGG | | | | | | Universal |
| 3483 | cg17819031 | CTGGAGGACACATGGACCACATTCATGAAGGCCCGGCTCAACTGCTCCCG | | | | | | Universal |
| 3484 | cg17905888 | CGTAGAGAATTGCAAATCTTGTCATGGCCACGCAGAACAGCTCAAGCATT | | | | | | Universal |
| 3485 | cg18093751 | GGGGAATAGGGCGTCCCTGGCCCACTTAGCTTTTCCCCCAACTCCTGGCG | | | | | | Universal |
| 3486 | cg18349747 | TTTCAAGGCGGCCATCTTAGTGCCTCAGCTCTGAGAAATATTTCTGAGCG | | | | | | Universal |
| 3487 | cg18419637 | AACAGCCACGCCCTCATGATATTTGTGGCTACTTTGGGAAAGATGCCACG | | | | | | Universal |
| 3488 | cg18481180 | CAGACTGCCGCTGTACTGGTAATTCCCCTCACTTGATTGTGTTACTAACG | | | | | | Universal |
| 3489 | cg18526971 | CGCCTCAAGAAGTGCCTCAAAGTGGGCATGAGGCGGGAAGGTGAATATTT | | | | | | Universal |
| 3490 | cg18535797 | GAAATGAGCGTGGCACATGTTGACATAGGAATGCATCCAGCCATAATTCG | | | | | | Universal |
| 3491 | cg18543660 | TCTAATAACCAGAAAATCCCCTCTGTGGGTCCAAAGGCTCTGGCGAATCG | | | | | | Universal |
| 3492 | cg18635807 | CGGTGCCATTGGGAGTCATGGAATTACTATTTATATGAGAGTTACCGAGT | | | | | | Universal |
| 3493 | cg18684142 | CGCCCGGCCACATGACCGGCGCCGGCCAATCGCTGGATTCAACCACTCAT | | | | | | Universal |
| 3494 | cg18787121 | ATTTCCTCACTTTTTATAACTTACTTTGATCCGGCCTCTCTTTGTCCTCG | | | | | | Universal |
| 3495 | cg18814344 | CGGGTCATCTTGCCTGTCCCGCCGGCCACCAGGCCCAGGCAAGCTGCCCC | | | | | | Universal |
| 3496 | cg18894136 | CGCCACCAAATCCATTTGGCTCTATTTAGGAACTAAGTACAGGAAGGTAG | | | | | | Universal |
| 3497 | cg18955753 | CTGGGGCACGAGGCGCTCCCTGGGATCACATGGTACCTGCTCCAGTGCCG | | | | | | Universal |
| 3498 | cg19017031 | CTGTGGGAACTGGAGGTAACAGTCTACAGCCATGGTCGCCCCGCAGCACG | | | | | | Universal |
| 3499 | cg19051359 | CGATGTATCTAGCCCTTGGCAGGGTGTCAAGGACAAGAAATAATCAGTCT | | | | | | Universal |
| 3500 | cg19054310 | CGCCTCCTAGATTAATGCAGAGAAAGCGGTATTGTCTGTTGAATGGATAA | | | | | | Universal |
| 3501 | cg19090691 | TGCTTCTCAGAGCGGGGGCTTTGTGCAGCAGTCTGCTCAACAGAGGGACG | | | | | | Universal |
| 3502 | cg19134574 | CGCCATTTCCCGCCTACCGACCTGCTGCACCGCACGGACAGGGACCCGGA | | | | | | Universal |
| 3503 | cg19195912 | CGCCAGATTGTGTCTCAAGACACATATGATGAACTGAACAAAATGACCTG | | | | | | Universal |
| 3504 | cg19291109 | CTTTTTCTCTAATGATCTAGTTCCTGTAGAACTGGAATGCTTATGTCTCG | | | | | | Universal |
| 3505 | cg19296119 | TGCCTATCAGAGGCTAATAATACTGCAGCAGCGTGTTAATTGCAAAGACG | | | | | | Universal |
| 3506 | cg19296935 | CGTCTTAATTTTTTGTAATCATTTTTTGTAAAGAGGGTATAATGTCAGGC | | | | | | Universal |
| 3507 | cg19319735 | ACCTTTTCCAATCCCGGGCTGTATATGGAGATTTGGGATTCTTTAAACCG | | | | | | Universal |
| 3508 | cg19342019 | CGGGCCCACAATCACTCAATATTTTAAGAGCTGTCATCACACCAGCATGA | | | | | | Universal |
| 3509 | cg19361075 | CGATGCCTAGAGCAGGAAGAACAGAGGATGTCATTTTCATTAATTTCTAA | | | | | | Universal |
| 3510 | cg19384462 | TAAAACAATTTTAAAGTGATTAAGTAGTTTAACACCTGTCTCCCGTCACG | | | | | | Universal |
| 3511 | cg19425332 | TCAATTCAAGCGCATGGCCTTGTGCAAGGAGCATGCTCCAGCCCTTCTCG | | | | | | Universal |
| 3512 | cg19513247 | CTGTGGCTGCTGCCTGCTGCTCCTCGCGGCGAAAAGCACAAAGCACAGCG | | | | | | Universal |
| 3513 | cg19573972 | TCCTCAGAGATGATGTACTTATGCAGACACACCATGTTGGGCACACAGCG | | | | | | Universal |
| 3514 | cg19584582 | CGCGTGTTCTTGTAAGTGTGACCGAACTGATTACGATGCATATGTTTAGA | | | | | | Universal |
| 3515 | cg19692957 | AATTACTCGCTTTATTACTTTAGCCTGGAGGAATCCTGTAAAAAACAACG | | | | | | Universal |
| 3516 | cg19756587 | ACAAAGATCCAACTGTAAAAAGTCTTACGTAACAGTTCAGGACTACTTCG | | | | | | Universal |
| 3517 | cg19812206 | CGGATGACAGAGTCCAACTTTCCCATTACAGCTGTTCGTTCCTTTAAGAG | | | | | | Universal |
| 3518 | cg19891210 | CGCAGGAAGAAGAAAGAATATGTCAAATGTCTTGAAAATCGTGTGGCTGT | | | | | | Universal |
| 3519 | cg19931377 | CGATAGTGCTTTGAAGTACTGGAGTGCCCAGTTCACATCAGGGCATGCGT | | | | | | Universal |
| 3520 | cg20023049 | CATGGCTACCTGGCTGGTTTCAGCGTGCGCTCAGGTCGCCTGGAGAGCCG | | | | | | Universal |
| 3521 | cg20025209 | CGGCAAAGGCGCCAGAAAAATCTGGAGAATTCATGGACACAGGCAGTTGA | | | | | | Universal |
| 3522 | cg20033770 | CGGCTGGCCAGCAAGGGCACGGAGCCTCACCCCACACCGGTAACCCTCTT | | | | | | Universal |
| 3523 | cg20083419 | CGTTCCTAATTGCACCGTGGAATAGATCTCAGAGGAAAGTAAATCGCCAT | | | | | | Universal |
| 3524 | cg20100200 | AAAGTTGCACAATACAGAAAACTGTTGCTCCATCTTCTACTACATAAACG | | | | | | Universal |
| 3525 | cg20194417 | CGGGGATCCAGTTACGGCTCGCTCATGACAGCCCATGGGAAATACCAGAT | | | | | | Universal |
| 3526 | cg20221627 | AGGGATTTGACCCATTCCGATTCGGAGTCCTCCCTCCACATGAGTGACCG | | | | | | Universal |
| 3527 | cg20306751 | TCCGCCATTTTGGCTGCCTCTGTCGGTCTGTTCAGTTACCACGTGAACCG | | | | | | Universal |
| 3528 | cg20334331 | TCACCTCGCCTAATCTAGCAGCTAATATTTATTAGAGCCAATTTATACCG | | | | | | Universal |
| 3529 | cg20345626 | GCGCTCTTTAGCAGACGGGCTTGCAAACTCTGGTGATAAATAGACTTTCG | | | | | | Universal |
| 3530 | cg20432508 | AATTCCACAAAAGCAAACCGAGTTGGCTGAGTCTCATCACCTGCCATCCG | | | | | | Universal |
| 3531 | cg20483513 | CGCCACAGCCAGGTCTGCAGCTCCTTCAGCAGCCAGAAGTCGTCCATCTT | | | | | | Universal |
| 3532 | cg20527060 | CCATTCTATATAGTGTATTAAAGTCATTTTCCACATGCTACCTCAGCACG | | | | | | Universal |
| 3533 | cg20583171 | CTCGCATTGTGACCCTTTGATATTAATTCAAAGGGCAATTAAAGAATGCG | | | | | | Universal |
| 3534 | cg20705693 | CGCAGGTCGTCTCCAAACCCAAAATTTCCACGTCGGCAAAAGTCAAGATG | | | | | | Universal |
| 3535 | cg20804050 | CGTGCGGCCCTGACTCACGTGCTTCCGGTTTGAAGGCAAAAAGTGTGCCT | | | | | | Universal |
| 3536 | cg20865948 | GTTCCACTGGTCAGCACTTCAATCTGGTATTTACAGAAATGGTGACTTCG | | | | | | Universal |
| 3537 | cg20867091 | ATAGGAGTAGCCACACCTCTTCTGTTACAAATCATTAAAGGTGTTGAACG | | | | | | Universal |
| 3538 | cg20884304 | CGAGCAAAATTGCATTTGTTTTGCAGATTTGCTCATCATTAAATGTGAAA | | | | | | Universal |
| 3539 | cg21030623 | AGACACCCACCTGTATGAGTACGCTTGTGGATCTTGAGGTTCTCGGAGCG | | | | | | Universal |
| 3540 | cg21089733 | CGATGATCATGTGGTATAAGAGAGATAGATGGCTTTTAGTTCCATCCTGT | | | | | | Universal |
| 3541 | cg21102477 | CGGTACGCGGGAGCCCTGAGCTTCTGCGGCTGGAATGAACCAAGTGTCCG | | | | | | Universal |
| 3542 | cg21180487 | CGCCTGAGCCTCAAGAGACTTGAGGTTGTTGGTGACATTCTTCAGCTCCT | | | | | | Universal |
| 3543 | cg21390586 | CGGGATCCCAAGAGGCACTGAGGTCAAGCTTCGCTCATTACAGTAGACAA | | | | | | Universal |
| 3544 | cg21509426 | CGAGGGCAAAAGTGCTTGATTTCTGGATACCACATGCTTTTCACATGATT | | | | | | Universal |
| 3545 | cg21590845 | CGGAGCCACAAGGACAGATGTAGGCAGCGTCCCACTGATATCATCTAGAA | | | | | | Universal |
| 3546 | cg21633990 | CGCTTGAATTCACTGAACCAGGAGCCCGGGTTTTCTTTGGGCCAAGAAGT | | | | | | Universal |
| 3547 | cg21703944 | TCTGGCCGTGGTAATACGTTCCCTGCGGGCAGCTGACTGAGGGAGAGTCG | | | | | | Universal |
| 3548 | cg21839725 | TGGGATTCCGATGAATCTCCTGAGGCCTCCCCTGGTCCTCCAGGCCCACG | | | | | | Universal |
| 3549 | cg21847954 | CGAAACAACTGTGGTGATAAATCCGCCTGCAGATAATTGGCAAGAAAACA | | | | | | Universal |
| 3550 | cg22040809 | GCCTGTCATTCTACCATTTGTCTCTTCCGACAGCAAGGTCTGGTAGTACG | | | | | | Universal |
| 3551 | cg22067905 | CGCCCTTCGAATACTTGGATAAAGGAGTCCATTTAGCAAATTACCTGCTT | | | | | | Universal |
| 3552 | cg22069272 | CGTCCTGTGGCTGAACCAGAGGAAGAGGGTGTGAGATCAGGGTTCCACTA | | | | | | Universal |
| 3553 | cg22144210 | GGTCCAGGTCTGAGGACATCACCTAAACAAAGGGCCACTCAGAAACTTCG | | | | | | Universal |
| 3554 | cg22146484 | CAGCCATTGTAGTGTATGCGCTGCCCCTGCAGCCCAACTTGCAGCTGCCG | | | | | | Universal |
| 3555 | cg22212874 | CGTTAATACTCTTAGACAACACAGATCTGAAATGGTGAAACCAGCAATTC | | | | | | Universal |
| 3556 | cg22234802 | CGCCTCTTTGGTGGTAGGTTTCGACCAAAAGAATTGGAAGAGCTATGATT | | | | | | Universal |
| 3557 | cg22338839 | GCAGTATGACGACGGCTTAAAAATCTGGTTGGCAGCACCCCGAGAGAACG | | | | | | Universal |
| 3558 | cg22354333 | CGCGGAAATTTGAAATATTTGGACAGGAAGAGAAAAGAAAAGAAACCTTA | | | | | | Universal |
| 3559 | cg22409694 | CGACAGGGAGAGTAGTAAAACTGCCCCTTAAGATGGCTTTTAATAGGAAG | | | | | | Universal |
| 3560 | cg22432362 | CGGTGAATCACCTAATTAAAATCAAGACATGAATTAAGCATCCATTTTTG | | | | | | Universal |
| 3561 | cg22661680 | CGTGAACCAATTGGAGAACTTTAGAAAATTGGATCAGCCTCAATAGTCAC | | | | | | Universal |
| 3562 | cg22703759 | TCTCGAGACAGCAGCATCAATCACCACATTTTCATTATTAAGTAACATCG | | | | | | Universal |
| 3563 | cg22710034 | TACCTGACCATTGACCCTGCAGGTAACTCACGTAGTGCTCCACCTACACG | | | | | | Universal |
| 3564 | cg22901671 | ACAGGGTCAATACACAGCCAAAAGCATATACTCAGGATTAACATATCACG | | | | | | Universal |
| 3565 | cg22914193 | CGTGGAGCCTCTGAGGAGGTGATGCAATTTCATTTTCATTGATCAGAAAT | | | | | | Universal |
| 3566 | cg22943590 | CACTTTAAAAGCTACATACCTAGTTCTTTTTAATATAGGCTATTCTTACG | | | | | | Universal |
| 3567 | cg23076498 | CTCTCTCATTCCCAGAGTGCATATCGGGAATAGACACACAAAGACATGCG | | | | | | Universal |
| 3568 | cg23089474 | CGCCCGGAGCCTCACAAAAGAACAGGATGTTGTTGATTTGTCACCAAACC | | | | | | Universal |
| 3569 | cg23120731 | CGCGAATGCCATGCCCAGTTCATTTCCTCTAAGGAAACACAACCTCGGGG | | | | | | Universal |
| 3570 | cg23148651 | GAGGTCAGATTTGGAGCTTCTCATTGCACGCGGAGATTATTATTGCATCG | | | | | | Universal |
| 3571 | cg23183090 | AGCCCCTGCATTGCAGAGGGACTGGAGATCTGTATGATCTGTTTATTTCG | | | | | | Universal |
| 3572 | cg23272565 | GAAAACTCACCCAGAGAAATTTTGCAAGCATATTATTTTCCAATGTTTCG | | | | | | Universal |
| 3573 | cg23287176 | CGACTTTCCGATTCACTGCTCCTGAAATAATTTTGTGTATTAAAACCTGA | | | | | | Universal |
| 3574 | cg23314464 | AAGATATCATAGGAGGGTCGCTGTTTGACGCAGCGCCGAGCTGACTTGCG | | | | | | Universal |
| 3575 | cg23344477 | GCTCTGAACTTTATTTTCCGAGTTTGAGGAAAATTATATTCCATCGATCG | | | | | | Universal |
| 3576 | cg23415880 | CGGCGTGGGTGGGGTTGACAAGAATAGAGTACATTATGCAGTTCATTTAG | | | | | | Universal |
| 3577 | cg23423329 | CGCCATGGGGTCCCCATCTCAGTCACTGGCATTGCCCAGGTGAAGCTTTC | | | | | | Universal |
| 3578 | cg23449696 | GTCACCCTTGTCACAGTTATTGAAATTCCGCAACTCGCCACACCAGGACG | | | | | | Universal |
| 3579 | cg23567910 | CGTTTTTCTCGAAGTTTAATTACCGTATGCTAATTTCCTTGCCTACTACG | | | | | | Universal |
| 3580 | cg23573900 | TTTCTTTTTGGGGGCTGGATGTGTTCCTGGCAGGGCCGATGATGGATGCG | | | | | | Universal |
| 3581 | cg23940242 | TTTTCCTCTCAGCTATCAAATGTCACTGCCTTGAAACGTGGGCCCTTTCG | | | | | | Universal |
| 3582 | cg23948922 | TCATCCATGCACTTTAATTGAAGAACTGCACTTTTTTTACTTCGTCTTCG | | | | | | Universal |
| 3583 | cg23998570 | TACATTTACTGACATTCATCGCATCTTTGACATTGATTATCTGATCAACG | | | | | | Universal |
| 3584 | cg24087602 | CGTGTCAGAATCTTTTAAACAGAGCATGGTGTTGCGTTTATTCGAACAAA | | | | | | Universal |
| 3585 | cg24104506 | TTGAAGGGTGTTAACCTACCCAACAGCCCGTGAAGAGTTAAGTAAATGCG | | | | | | Universal |
| 3586 | cg24393114 | CATTTTGAGAATTGCATCAGACTAAAACTGATTACAGATATTAAGCAACG | | | | | | Universal |
| 3587 | cg24434315 | CACTGAGAAGTTTACTGATTTCTCCCAGTGGAATCTGCTGGAATTTATCG | | | | | | Universal |
| 3588 | cg24551749 | CGGTGTGAAATAACGGGCCCATATAAATCCCTCTGCCGCCCGCCTGCAAG | | | | | | Universal |
| 3589 | cg24557436 | ACGCACCTGTGATACTGCGCCTGCAGGAACTGGAATTCCTCTTTAATCCG | | | | | | Universal |
| 3590 | cg24580312 | CGCTCTTTAAATCTGGATATTAGTGCAGGAGAGACGGTCTTGTAGAATTC | | | | | | Universal |
| 3591 | cg24597392 | GTAATTTCTCCCTGGCCCTGTCTGCATCAGCACTATTCTCGAAAGTTACG | | | | | | Universal |
| 3592 | cg24615318 | CATTAAAGCCACTTTTCCTCTCAGCTATCAAATGTCACTGCCTTGAAACG | | | | | | Universal |
| 3593 | cg24927890 | AATATAGACGGCTAATGTCTTCAGTATGTATGAAATATCCCATATGCTCG | | | | | | Universal |
| 3594 | cg24952195 | TTAATTTGACACCTTCTGTTCTAAACCCTTTGTTTCTATTCAAAATCACG | | | | | | Universal |
| 3595 | cg25047480 | CGGTCACCCGCAGCGCCTGAAGAGGCTTCCCGTCCAGAGTCACCTCCTCC | | | | | | Universal |
| 3596 | cg25055739 | CCCAGACTATTTCCATCCTCTGATTCTCATTCCAAGTGCATCACTCGACG | | | | | | Universal |
| 3597 | cg25108269 | CGCTATTTTAACTTCAAATACTGAAGAACGCCTGATAAGGCTGAAAAGAA | | | | | | Universal |
| 3598 | cg25184118 | CGGCCCCCCCCTCGGAGAACAGTAAGGACCCTACTGGAGAAGCAGAATTG | | | | | | Universal |
| 3599 | cg25184464 | CTTTGATTTACCTTCTTCGAATGCCAAATGGAGACAGGTGATAAAGAACG | | | | | | Universal |
| 3600 | cg25195795 | GTGGAATATCAATTTTTATTGCATCACCTGTCAGGAGTGTCCAATCAGCG | | | | | | Universal |
| 3601 | cg25229850 | CTAGTTCAACCATGTCCTTGTTTTATTCTCGAGGAACCACCTGTGAAACG | | | | | | Universal |
| 3602 | cg25278124 | GAGCTGCACACGGCACTGGAAGTCACCACCAGGCCCTGGGGCTTCACACG | | | | | | Universal |
| 3603 | cg25368206 | CGGCATGTGGAAATTGACTTCCCTTGATTTAAGCTGCTGTGTCTTTTCAT | | | | | | Universal |
| 3604 | cg25410683 | TCTATGATCTACCTCAGGGGAATGGCTTTTGCCTCGGACAGCTCAATTCG | | | | | | Universal |
| 3605 | cg25467897 | CGCTAAGTCCTCGCCCTGTTTCTTAGCGCCGGATCCTGGGGACCAGACAC | | | | | | Universal |
| 3606 | cg25518911 | GGGGCGCGAAGGCCGCGGGGAAACGCTTGTGCAGGCGGTAGTCCTTCTCG | | | | | | Universal |
| 3607 | cg25615279 | CGCGTCCAAACTTTAAAATAAGGAAATACCAGAGAAAATGATCTCAATAA | | | | | | Universal |
| 3608 | cg25622623 | CGCCCCCGGAGACCCACAATTGGTCCAAAAAGCGTAAAATCAGCAATCAA | | | | | | Universal |
| 3609 | cg25639084 | CGAGAAGGTTAAATAAATAAGAGAGAAAGAACACCCTTGGCAGAAAGCAC | | | | | | Universal |
| 3610 | cg25649550 | TTCTAATGCTTTCATAAATCCAGTCTCTCCATTATGTGGAATATCAGTCG | | | | | | Universal |
| 3611 | cg25662803 | CGTCGTTATCTTTTGCAAAATACAGCCCTGGAGATCTTTATGGCAAACAG | | | | | | Universal |
| 3612 | cg25687751 | GAGCAAGCAGGAATACTACATCCGGGTCCTAATGTCCATTGCCATTTGCG | | | | | | Universal |
| 3613 | cg25694286 | ATCCCGTCTCTTTGCCTTTTATTTTTGGCGGCCTCCTTTCGAAAACTCCG | | | | | | Universal |
| 3614 | cg25699407 | CGAGAAGGTTCACACACCCCCTCTATTCATTCCTCTTCCACAGGTGTGCG | | | | | | Universal |
| 3615 | cg25771548 | GCAGCTCCACTCCTGAAGACCTTCAGCTTCTTGGGCTTTGAGATTGTACG | | | | | | Universal |
| 3616 | cg25805807 | CGCCAGGCTGGAGCAGCCCAGCCACAGAGACCTGGGCATAGAGCACTGCA | | | | | | Universal |
| 3617 | cg25950871 | CGCACGCGCTCCCATATAATTTATGACTGCCTCTGGTCCTGATGACAGAT | | | | | | Universal |
| 3618 | cg25960060 | CGCGTTCACATAATACGAAGAACTCATAATTTTGACCTGTGATTTGTTGT | | | | | | Universal |
| 3619 | cg26032884 | GAGGCTCTCCAGCGCCACCTTGGGGTCGTGCAGGAAGTGCGTGGTCTGCG | | | | | | Universal |
| 3620 | cg26045960 | GGAGCTGAAGCTGATGGCTCCTCGGCGGCTGAGGACGTGCTGGGGGACCG | | | | | | Universal |
| 3621 | cg26062663 | CAAAGGACCCTCCAACTTAACCAGCGTCATTGTCCATTTAACCCACATCG | | | | | | Universal |
| 3622 | cg26102159 | GCTCTGTGCACTGAGAGACTGTATCCCCTCAGTTGGCAGGCAGAGCTCCG | | | | | | Universal |
| 3623 | cg26234822 | CGCTCAGATGCAGCAGCACCATGTTCACGAGAGTACATGGGCTGTCCTAT | | | | | | Universal |
| 3624 | cg26275174 | CCCAGCTCTCTTTTTTTTCCTGATCAGCTGATGAAGAGACTAGCAGCTCG | | | | | | Universal |
| 3625 | cg26363341 | CGTGAACTTCCATATCAAGAAGCCCTCCAAGTACCCACATGTGCCCCCTC | | | | | | Universal |
| 3626 | cg26420776 | CGGTAATGTATAATAACCCTTTGGGCAAGAAACCCAGTCCCCGCAGCAGC | | | | | | Universal |
| 3627 | cg26474314 | CGTTGATTATGTTTCGATTTTCGTTCAAGGGCCTCTGCTGTTCGTTCTTT | | | | | | Universal |
| 3628 | cg26581729 | CGCCATAAGTGCCCCCAGAACTTCAGCGCCCACCATGGCGCACAAGGCCG | | | | | | Universal |
| 3629 | cg26598122 | CGGGAATCCTCTCCAAAGAAGGAGGAGACTGTAGCAAGTAAGGCATAGAG | | | | | | Universal |
| 3630 | cg26603143 | CGCTGTTTGGATTTCCTGATTTTATAACAGGGGGGCTGGTTAATATCTCA | | | | | | Universal |
| 3631 | cg26616379 | ACAACCCAGTTTAAATAAGAGTCTCTGAAACTTCGCTCTCAGCCACAGCG | | | | | | Universal |
| 3632 | cg26781330 | GTAAACGTACCTGTCCACAGCCCGGGGCATTGCACACAAACGGTCTGTCG | | | | | | Universal |
| 3633 | cg26845448 | CGAGAGCAACCCCTTCACCGAAATAGCCATGAGCAGCTGCAGGTACAACG | | | | | | Universal |
| 3634 | cg26885075 | CGGTTCACAGACCTCTTGAAGGAAAGTTTGTTTTCCACTCATAGATGAAA | | | | | | Universal |
| 3635 | cg26920416 | CGGGTTTCCAGTGGGCGCATCAGTTACAATGACATGTTTGAGATGCTGAA | | | | | | Universal |
| 3636 | cg26957053 | CGACAATGACAGCAGCATCTGGAACTACCCGCCCAACCGGGAGATCTCCA | | | | | | Universal |
| 3637 | cg26989456 | CGGACCACCAGTTTCCAATAAACCATAAAAGGCCGATCAGCAGCCATTGT | | | | | | Universal |
| 3638 | cg27035369 | TGCAATCAGTTTCCGAAAGGTCAGCCAAGGTCCTCCGGCTCTCGCAGACG | | | | | | Universal |
| 3639 | cg27096599 | GAACAAAATCCACCAACACGGTTCCAGTAACCTCCGCATGTCCGAAAACG | | | | | | Universal |
| 3640 | cg27208859 | GGTTGTTTATGCACACACAAGGCCCTTTGAAAATAACCCTTTTCATGTCG | | | | | | Universal |
| 3641 | cg27242082 | CAAGTGGAACAGAATTCCCTATGGTCAAATTAAGTGATCTCTTTATTTCG | | | | | | Universal |
| 3642 | cg27275508 | CGGGGAGATAAAACTCTCATAGCCCTAACTGTCAAATAGGGCCCTTTTCA | | | | | | Universal |
| 3643 | cg27291660 | CGCGGATGGAAGACAGGCATGCAGAGGAAATCAAACCTACAACCGCGACA | | | | | | Universal |
| 3644 | cg27294582 | CGGTGATTTACTTCCCTGCAAATGAGTTGTTTCATATTTTGCACTGTCTT | | | | | | Universal |
| 3645 | cg27492942 | CGGATGTTATTTGTACGAAGGGCAGTTCGTAAACAGCACTTGTTCTTTTA | | | | | | Universal |
| 3646 | cg27547112 | TTTTCAGGTCCTGCAACACCATACCCACACTGCAGCCATTTAGCATGTCG | | | | | | Universal |
| 3647 | cg27593991 | GTGATTTTGCTAAAATGCATCACCAACAGCGAATGGCTGCCTTAGGGACG | | | | | | Universal |
| 3648 | cg27637521 | GGAAACTTGCTGTGGGTGACCATGGCGCACGGAGCCAGCGTGGATCTGCG | | | | | | Universal |
| 3649 | cg00101675 | TCCTAAAGAAACTTCAGCAGTGCTGTGTCATTTTTGACTTCATGGACACG | | | | | | Universal |
| 3650 | cg06259996 | CGGCCAATTCACCTGCGAGGGCTGCAAAAGTTTCTTCAAGAGGAGCGTCC | | | | | | Universal |
| 3651 | cg20146241 | TTCACTTTCACAGACATGAACCACCACGCTGGGTGTCGACTCTGTTCCCG | | | | | | Universal |
| 3652 | cg03747401 | AGCAGCCATTAGCACGTCATTGGTAATCCCAGCGACACCTTTCACATCCG | | | | | | Universal |
| 3653 | cg15403305 | CGGAGCCTGCCTCCAGACTGTTGGCGTTCACGGCTCTGCCTCGCAGCCTC | | | | | | Universal |
| 3654 | cg20236179 | CGTGCCCACGGTCACCGCGATCACAACCAGCCAGGACCTCCAGTGGCTTG | | | | | | Universal |
| 3655 | cg01910711 | CTCAGAGCACAATTGCGTTTCCTGCGGGTTATTTTTGGCGTGGGAACGCG | | | | | | Universal |
| 3656 | cg24312350 | CGCGCTGCAGCCCCCCGCTCCGGAGCGGCCACTTGTCCAGCACCTTTACC | | | | | | Universal |
| 3657 | cg08180952 | GTGGGGGGGATTTCCCGGGTAACAGAGAAGCGGCCGCGGCGGTAGAGGCG | | | | | | Universal |
| 3658 | cg10040836 | CGGCAGTACTGGCACTGGTTGCGATGGTGCTGGTCGATGGGACAGTTCCG | | | | | | Universal |
| 3659 | cg11686722 | CGGACTGGGATATTAAATATACGACACATCCAGGAGTTTATTGGAGCGCA | | | | | | Universal |
| 3660 | cg18418719 | CGTGGCCGCCATATTCAACGAGGCCGTCCTCGCAAATACTGCCCCGATGA | | | | | | Universal |
| 3661 | cg07727941 | CGCCACCATCGACCAGTGCCCCACGCGCATCGAGGAGACCTCGCCCATCG | | | | | | Universal |
| 3662 | cg25066947 | CGCGGGGCGCCCCATTGACTGGGCCCCGAGCCCCACTTTTCACAAACTCC | | | | | | Universal |
| 3663 | cg02828846 | CAACACCCACCGGTAAGAACAGGAGGTATTTATATCCAATTTAATTATCG | | | | | | Universal |
| 3664 | cg22844979 | CGAGGATTTTATAATGGCATATTTTTTCAGTATTAAAGCGAAAATGTTTT | | | | | | Universal |
| 3665 | cg15229894 | GACGAAGCGGCTCCTCTGAACCCCGGGATGTACAGTCAGAAGGCGGCTCG | | | | | | Universal |
| 3666 | cg02499612 | CGGCCCATCTCCGACCGCTTCCTGATGCGAGTCAACGAGTCGTCCTGGCA | | | | | | Universal |
| 3667 | cg18468088 | ACCAAGTCCTGTAACATGCCTGGTATTCCCAGTTATCCTCACCTTCCGCG | | | | | | Universal |
| 3668 | cg04186556 | ATTTCCTAGCAGGTTTTCACGGACCCTAGCAACCTTCAGCGGCACATTCG | | | | | | Universal |
| 3669 | cg17201550 | CGGCACCTCCATGGTCACCGGGATGATGAGCGCATCCATCTTGGGCTCGT | | | | | | Universal |
| 3670 | cg18573296 | CGATGGAGTCGTATGATGCCCTTGCCTTGTTTTATATTGGCTGTCAGCGC | | | | | | Universal |
| 3671 | cg27495736 | TTCCCACAGTAACAATCGAAAGCCACGGTTGCCCTGGAGACGCGGGGGCG | | | | | | Universal |
| 3672 | cg03708116 | CGGACCCGACACACGCTCACCTGGCTGAGGCCCCCGCCCCCCACAGGCAG | | | | | | Universal |
| 3673 | cg00900734 | CGCCATTCGACACAAGCCGCTGAACATGTCATCCCGTTTTTCACCCAGGG | | | | | | Universal |
| 3674 | cg00535482 | CGGCTCGCGCTTTGTATACTTTGCACTTGAAATCGTTACATTCAACTGAA | | | | | | Universal |
| 3675 | cg13496662 | CGCCAGTGACCCTGGCAGCTGGTGGGGAGGGTGTCCTCCGCCTAGCCTCA | | | | | | Universal |
| 3676 | cg02041553 | CGGCCACCCCTCGCACCACGTACCCCGCGGCCTCTTTGTTGTTGGGCACC | | | | | | Universal |
| 3677 | cg22113843 | CGCCGCCATTTTGTGTCCCCCTGTTGTTGTCGTTGACATGAATCCGACAT | | | | | | Universal |
| 3678 | cg25210084 | ATACAGCGCTCAGCTTCATCCTTGTTGGATTCCATGGCGGAACCAGAACG | | | | | | Universal |
| 3679 | cg12756474 | CGCCATTTGAAATGCTAACACAATGAATATTTTCTCATTGGTCTGTGTCC | | | | | | Universal |
| 3680 | cg01486146 | CCCATCAAAAAACTTCTATTAGTAATGGAGACCTCGCCGGAGACCTTCCG | | | | | | Universal |
| 3681 | cg13909487 | CTCGCCTTAATCAATTAGTTCGTGATTTGCTGCAGACCCCTGTCTCTCCG | | | | | | Universal |
| 3682 | cg12053353 | AATTATGCTGAAATAAAGGCCATTCAATTTGCTACAATCAGTAACTTTCG | | | | | | Universal |
| 3683 | cg13233081 | TTTCTTCTAGCTACTTAAAGGCATTACTGAATTATGGGCATCACAAAACG | | | | | | Universal |
| 3684 | cg18141557 | CAAGATGGTCCAGAGCGACTTCATGCAGGGGGCCATGGCCGCCAGCAACG | | | | | | Universal |
| 3685 | cg15741341 | CGGCCTGAAAAACACCTCTGTCATGATGGTGGATCTACTCGCTCACACAA | | | | | | Universal |
| 3686 | cg27072792 | CGGGGCGAATGGCTGGCAATCTTACTGGGATTACAGAACAAAGAGCCTCC | | | | | | Universal |
| 3687 | cg26312920 | ACATTGATGCCAACTGCCAGAGCTGGTACCCATGCCATCTGCTAGTGACG | | | | | | Universal |
| 3688 | cg01846532 | CGTTACATGGCTGGTTGCAGCCTCAGATTAATGCGAAGACCCTGGCGATT | | | | | | Universal |
| 3689 | cg21132526 | CGACAGTTCAGCACAAGATCAGTGACATTCTGTCTCATTAGAACCACGCT | | | | | | Universal |
| 3690 | cg16554811 | TTACTTTTCAGTCCCTATTTGGAACTGCTCTCGCGGCAGTTCAGACCTCG | | | | | | Universal |
| 3691 | cg00650422 | AAGATCACCGGCCTCTCCCTCACCCAGGTCAGCAACTGGTTCAAGAACCG | | | | | | Universal |
| 3692 | cg26589497 | CGCAACTCCATCACCGTGCCCTACAAGGTGTGGGCCAAGTTCGGACACAC | | | | | | Universal |
| 3693 | cg07373156 | CGGTTTAATTGCGCCGAGTTTTAAAAGGTCCTATAAATGTAATTGGTATT | | | | | | Universal |
| 3694 | cg11214667 | GCATTTTACGAGTTCTTTCCTCTACAGCTATAAAACTGCAGCTTCGATCG | | | | | | Universal |
| 3695 | cg11407867 | CGCCCTTCATCTAGGTAGGGGTGGGCTAAGGCATCCTTAGCGGATATTCT | | | | | | Universal |
| 3696 | cg16367356 | CGGGCAAATCCCTGCATCATGCTGATTTCCAGTTTTGGAATTGATACCAG | | | | | | Universal |
| 3697 | cg16981766 | GCTTATTGTCAACCTCAGAGAAGTGTATATGTTTCCCTTCATACATCACG | | | | | | Universal |
| 3698 | cg11864223 | CGCTTGACACTCACACTTTGTCCCTGACTGACTATGCTTGTGGTTCTCCT | | | | | | Universal |
| 3699 | cg14397160 | ATCAAATTCATGTCGTAATTAAAGCTGTCAGTTGGAAGGCAAGCCAATCG | | | | | | Universal |
| 3700 | cg06799948 | CTCCAAATCAGACTTAAGGATTGTTTTATTATTTAATTACACAATCATCG | | | | | | Universal |
| 3701 | cg01938132 | ATTGCTGTCTTTTGTAATCCATCCATGCAGTCGCACCAGAGCGATTAACG | | | | | | Universal |
| 3702 | cg26736130 | CGACATTTAATAGTGCTAATTGCACTCCAGTTAAATTGCCCTGATTGCAG | | | | | | Universal |
| 3703 | cg14412072 | CGGCTGCCATTAGGGAAATGGGCATCATTCTTATTGATCCAGCATGGGCT | | | | | | Universal |
| 3704 | cg15810562 | CGCTTGAATACAATTTGTGTAACCTTGGCCAAGACAGCCTGTCATTTTAA | | | | | | Universal |
| 3705 | cg17041137 | ATCGCACTTCTGGATTTAATCAACTTTTTTATCCAGTGTTCAGGATGTCG | | | | | | Universal |
| 3706 | cg15686078 | CGGGGCTTGAGGGTCTCGGGGCCCCTCGCTAATGTAAGGTTATTGTAATT | | | | | | Universal |
| 3707 | cg24148137 | GGCTGCAGGATGGGCTGTATCCAAAGCATCACCTGCAAGGCGCGGATCCG | | | | | | Universal |
| 3708 | cg23128382 | CGAGAGGGCTGGCTCTTCAAATGGACCAATTATATCAAAGGCTACCAGCG | | | | | | Universal |
| 3709 | cg18169540 | CGCATGGCCATGGGAAACCCCAGTGAGTTCTTTGTGGATGTGATGTGAGC | | | | | | Universal |
| 3710 | cg11254527 | CACAAGTATAAGATACTGTCTGGATCATATTTTTCACCATTTGGTCTCCG | | | | | | Universal |
| 3711 | cg11672180 | CGGGACAGATAAAAATGAGCAGCATCATATTGTTTGACAGAATGATCTCG | | | | | | Universal |
| 3712 | cg26472530 | AAGTTAAGTTCCTGCGGACGCTCCTCTTGAAGAAACTTTTGCAGCCCTCG | | | | | | Universal |
| 3713 | cg26659116 | AACAAGGACATATTTTATTGTGCCCATGTTGTGGCACTGTCTTTATACCG | | | | | | Universal |
| 3714 | cg26879535 | CTACTATTGCTTATTTCACACTTCAGTAGGTAAAGATGCATTAATAATCG | | | | | | Universal |
| 3715 | cg12394655 | TATGAAGACCAATACACTCATACAGCTAGATAACCAAAGATAACAACTCG | | | | | | Universal |
| 3716 | cg07069838 | CGCAAGTGCTAATCAGCTCCATTGACACACACAGGCTGGGGCTACAATCC | | | | | | Universal |
| 3717 | cg07265990 | GAGCTGAAGTTTGTTTCTCCTTCCATCATTTCTTCCTCTTCTAATGTTCG | | | | | | Universal |
| 3718 | cg05544360 | TAAGAAATTCAGTCACAATAGAGAGCCTAGTTTTGAACATGTTTCACTCG | | | | | | Universal |
| 3719 | cg09108310 | CTGTTTACCTCAAGTTTGAATTTTATCTGCGCAACATTAGCTGGAATGCG | | | | | | Universal |
| 3720 | cg09784081 | CGCAGATAAAATTCAAACTTGAGGTAAACAGAAGAAGAGGAGGAGGAGGA | | | | | | Universal |
| 3721 | cg10042482 | CGGCCAGGCGCCAAGAAATCAGCCCCAATTACTATATTCACATTGTCAGT | | | | | | Universal |
| 3722 | cg13301933 | GTCGGGGGCTGCCGGAGCTGACTCCATGTTTTTTCCCAATGTAGAGATCG | | | | | | Universal |
| 3723 | cg16697952 | CCCAAGGCGATTGTCATATGATAGCTAAGAAGTGGCACATTAATGAAGCG | | | | | | Universal |
| 3724 | cg02681015 | ACTTACTTTTGGCTAGCTTCCTCGCCCTCGCCTTGGATCGCATGGTGTCG | | | | | | Universal |
| 3725 | cg09557545 | CGAAGCTCCCTGCTGATCGATTATTTGATAATTGATTTTCCCCGCAGCCA | | | | | | Universal |
| 3726 | cg07774376 | AAAGATGACCCGAAGCACAAAGCAACTGACAAACAAGTCCGGACAAATCG | | | | | | Universal |
| 3727 | cg01797373 | CGACTGAGTCATTTGTTTTGATTACCCGGGTGATTGCCAAATGTTAATTT | | | | | | Universal |
| 3728 | cg21102110 | CGGCCCATAAACTAGAATAGCTATGAAACACAGGCTGCCTTTATGTTGAA | | | | | | Universal |
| 3729 | cg21197642 | CGGAGTGATAGATACCCATAACTCAGAGTCCTTAATTGTACTTAATTTGT | | | | | | Universal |
| 3730 | cg16925789 | CCACTTTGCCAAATTAATTTTGACTCCCATAATTATTTGCTGTAGGAGCG | | | | | | Universal |
| 3731 | cg18679961 | CGGAGCCAAGTAACTCTATTTTATTATATTTGCTGGGAAATGAACTAGCT | | | | | | Universal |
| 3732 | cg18743610 | TGTTCTGCAGCAGGGCCTGTGTGAGAAAACAGCCTTTGCTTTCTCCTACG | | | | | | Universal |
| 3733 | cg19271316 | CGCAGTGACCCCCCTGGATTGTTATTAGGCCGGTTGGACATTGGCACCTC | | | | | | Universal |
| 3734 | cg20059238 | CTTGCCATTGCTGGTCAGCAATAATCGAACCACTTTCTGGGGATTAAGCG | | | | | | Universal |
| 3735 | cg20391664 | CGGGTTATATATTTTAGAGAACCTCATTAAGTGCTTTTTAAAATGAAATT | | | | | | Universal |
| 3736 | cg20621276 | CGCCTTCCTCATCGGCTGCATGTTCATCAAGATGTCCCAGCCCAAGAAGC | | | | | | Universal |
| 3737 | cg24080303 | CCATGATGTCGTGAAAATGCATGTTGACAGCCTGGTCCACTGATTGTTCG | | | | | | Universal |
| 3738 | cg16816020 | TATTTACTTTTCTCATAAACAAATCAGCTTGGGTGCCGTCAGAGCTGACG | | | | | | Universal |
| 3739 | cg16960327 | CAGCTAGACCCGCCCTGGTTTCTACCTCCGAAAGAGTCAAGGCTATTCCG | | | | | | Universal |
| 3740 | cg23692250 | CGGTGCAGAACATGCTATAGTATTGTAGTCATTTATGTAATGTCATTGAA | | | | | | Universal |
| 3741 | cg09642734 | CGGCCCGGTTGGCACCTACCCACCAGGATGCTGGCAGTCCTGCCCTCCTC | | | | | | Universal |
| 3742 | cg25072946 | AACAGCAGAGAAATAAATTAAGGGATGCAGTGGCCACCACCAGCCAAGCG | | | | | | Universal |
| 3743 | cg25172570 | CGGGCTCTTTGGTAACAAACCAACCCTGACTTTAGGAACCAATACAAACA | | | | | | Universal |
| 3744 | cg00493843 | CGGGCTCTTTCATGCCCCATCAGAATTGTAAATGAGGGGTCTGTGGGCAG | | | | | | Universal |
| 3745 | cg00935831 | CGGCGGCTGAAATTGAATTGATAACTGCATCAACGTGCCAGATAACGCCT | | | | | | Universal |
| 3746 | cg02270238 | CGGGAATTTATTTGCCAGGAAGGATGATCCCATCATACTGAAAGAGAAGA | | | | | | Universal |
| 3747 | cg14472711 | TTACAAGGTCCTTCTTCCCCGTAAAACTTTGTCCAAGTACCTCATGTACG | | | | | | Universal |
| 3748 | cg00399596 | TCCCTTCCATCTTGACTTTTGCCGACGTGGAAATTTTGGGTTTGGAGACG | | | | | | Universal |
| 3749 | cg05207134 | CGCTGTAATCTCTAATATGTTTTTTTCTAGGTGATGCTGTCAGATAATGG | | | | | | Universal |
| 3750 | cg23274273 | CGCCGACAAAGAAATCTTGCATGGAGACTACAAGTCTGGAGTTTCTGGGA | | | | | | Universal |
| 3751 | cg16701192 | CGTCACCGGGGGGGTGATGTAATGCACTCTAAATAGAATGTATTGTAATC | | | | | | Universal |
| 3752 | cg16848740 | CTTCATAAACCTTATATGCTTATAAAACAGCATATAAAAATTTAACAGCG | | | | | | Universal |
| 3753 | cg17455697 | CGCCTGCAATCCCACAATCAGTGTGCACTGTAATTGTCGAATTTGATGCT | | | | | | Universal |
| 3754 | cg22454306 | CGTCCCCCCAAAGACAGGGGACAGATGTTTAACATCAGCCTATTAATCCC | | | | | | Universal |
| 3755 | cg16986738 | TCCCTGGAATGTTTTTGACTTCCTCATCGTAATTGGCAGCATAATTGACG | | | | | | Universal |
| 3756 | cg25326872 | ACCCATAACTCATTAGTGTTGAACTCACGAGGTATAAAGACCAACGCACG | | | | | | Universal |
| 3757 | cg26748472 | CGCCCTGAAATCTATTTAAAGAGAGGGAAAGGGGAGGAGAACAAAGGGGG | | | | | | Universal |
| 3758 | cg07978738 | CGGGCTGGTGGCACCTGTCCTTGTGTCTCGTGGTATTTACTGTGCTACCT | | | | | | Universal |
| 3759 | cg02007453 | CGGGGCTGTTGAGACTTTCTGGCATGGTGGTCATGGTCATCTCTTCTCGC | | | | | | Universal |
| 3760 | cg02471195 | CGGCCTATTTGGTATGGTCTTTTTATTGGAAAATGACAAAGGGGTTATAG | | | | | | Universal |
| 3761 | cg12945551 | CGTAATGGATGAGATATTTACAGCAAAGCTGTTAAAACATTGAATAAAAT | | | | | | Universal |
| 3762 | cg18851565 | CGCTGCATCTTCTGCATGTGCCACTTTTGGGAGGACGTTTGAAACTTACT | | | | | | Universal |
| 3763 | cg07800922 | CGACTCCCTTGATAAATGGGACTTCCAGACCACACTTTATTGAGTCTCTG | | | | | | Universal |
| 3764 | cg10707234 | CGTGTAGAAGCGAGTCTGCTCAACAGCTTGTTATTTGGTGGATTGTGGCA | | | | | | Universal |
| 3765 | cg12648670 | CGACACGCTCAGCTGCATCTTGTTAAAAGCTTCCTGCGGGTTGGCAGAAT | | | | | | Universal |
| 3766 | cg13959241 | AACCCCTTATTTACATTTTGCGGTTATTTTGAGCCTTTGCCATCTGTTCG | | | | | | Universal |
| 3767 | cg05901294 | CGCCCGAAAGCCCAACAAGAAAATTTGTAGGAAAGGAGGTAGGTAGCGAG | | | | | | Universal |
| 3768 | cg19691778 | CGCAGACGATCTCTCGCACACTCCTCTCCGGAGACAGAAGTATTTGTTTG | | | | | | Universal |
| 3769 | cg14023941 | GAAGATTAAATTAAGTATCATATCTGCTGACCCTATTGCCATGGTTACCG | | | | | | Universal |
| 3770 | cg08522676 | CGCCCTTGTCTAGACTGGAGACGACACCTCCTCTGTGTGTAAACAGAGGC | | | | | | Universal |
| 3771 | cg09010707 | CGTGGGGCCAGGGCTTCCTGGGTTCAGCTACCAGCCGAGACTCTGAGTCA | | | | | | Universal |
| 3772 | cg09939922 | CGAGCCATGATAGAGAACTTCAATGCCAAATATGTCTCCCTGCATGTCAG | | | | | | Universal |
| 3773 | cg10426441 | ACTGCTTTGACCCAGAAGCCTGGGATATGCTGGATGATGAGGTCTCTGCG | | | | | | Universal |
| 3774 | cg15600660 | ACCCACAGATACAATGGGTACCTTGAACTCGCTCAATTGATGCAGGCCCG | | | | | | Universal |
| 3775 | cg06807725 | TTAACTCCTCCTTCTGTTGTCAGAAGAATGTTATTCCCCTTCACATCACG | | | | | | Universal |
| 3776 | cg07071132 | AGGAATTTACTGCAATTCCTTGCGGATTTTGCACATACAGCTCGCAAGCG | | | | | | Universal |
| 3777 | cg04902782 | ATCTATTGTTGCACTTCATAATGAGAAGGCAGATTTACTGCCTTTTACCG | | | | | | Universal |
| 3778 | cg09716123 | TCCCCTAATCAGGATGGAGACGCAGTTCATTACAAGCTGGTGAGGCAACG | | | | | | Universal |
| 3779 | cg11124915 | TGCCAGCAGTCTTGGGATAGTGCTGAGGTGCACCCCTACTTCCTCCTCCG | | | | | | Universal |
| 3780 | cg12591580 | TCCTATAATGATTACCTGCGGGCGGCTCAGATGCACTCTGGCTACCAGCG | | | | | | Universal |
| 3781 | cg08375909 | CAAGCAGTGTGACCAAAAGAAAATATTCCAAAGCTTGTCACGTATGACCG | | | | | | Universal |
| 3782 | cg09462662 | CGGCCGCCAGCCCAATTGGCAGCTCTCCATCGCCACCAGTCAATGGTGGC | | | | | | Universal |
| 3783 | cg05052242 | CGGGTAATTGGATTTATCTGGTTTTAGAATTTGTTAGGTTCTGTTGCAAA | | | | | | Universal |
| 3784 | cg06059859 | TAAACAGGCACCTCCGTCCCTGACCTACTTTCTGCCTTCCCTAAATGTCG | | | | | | Universal |
| 3785 | cg00422680 | CGGTGTTATTGTGATTTAAATCCAAGCTCTATAAAAGAGAGCAGTTCTTT | | | | | | Universal |
| 3786 | cg14116948 | CGCTGTGAGCCTCAACACCACAGGCTGCAGTGCTCTGCGACAGAAAAACA | | | | | | Universal |
| 3787 | cg08396875 | GATGCAGCGATGGGCTGCACTGCCAGCATTGTTCTGCTTCAAGCTTTTCG | | | | | | Universal |
| 3788 | cg27052685 | CGCTTTCCACACGTGGCAAATGATTGCCTCGGTAAAGGTATTTTCTGTTT | | | | | | Universal |
| 3789 | cg25530799 | AAACATGTGTTTTCAGGATTTTCTTTTCACATGCCACACAAAAAGTAACG | | | | | | Universal |
| 3790 | cg17820491 | CGCCCTGCTGGAAAAGAAGAAACTCATGGCCGCTAGGGAGCGCAAAGCCA | | | | | | Universal |
| 3791 | cg11458204 | CGACTTTTGTTTTAATAACAAAGTTGTTGATTTTCTACTAAGGGTTCTTT | | | | | | Universal |
| 3792 | cg08160490 | CGCTGGGAAATAGATGTTCTGTCTCTATGACAATATATTTTAACTGACTT | | | | | | Universal |
| 3793 | cg10013179 | TCCTGGAAGGCTGTCTGACATCACCCCGTTTCTTGTCGGCTGATGTTTCG | | | | | | Universal |
| 3794 | cg25073174 | CGGCAGGGTGTGGAACCTCGACATGACCCCCAGCTGTTCTTTGTAAGGGA | | | | | | Universal |
| 3795 | cg25468783 | CGGGCGATGTTAACATCTCATTATCTTTGTTACTGTAATTACATTATCCG | | | | | | Universal |
| 3796 | cg26154615 | CGGGTTAGGAGGCAAGCACATTACAGCTCCATTTTACCTCTGCAGGCATC | | | | | | Universal |
| 3797 | cg09176584 | CAAAGGCTAATTTGGTTACCCTCCGGATCCACAATAGCGAAACTGTGTCG | | | | | | Universal |
| 3798 | cg07499079 | GAGAGGTCCAGACTGGCTAAATCGTTGCTTTGGTTCAAGCAAGGCACTCG | | | | | | Universal |
| 3799 | cg10324939 | GGTCGTGGTAATTTACATCCTCTCTGTGTACATTAACTTATCTGCAGCCG | | | | | | Universal |
| 3800 | cg06158983 | CGACTTGGTCGAGTAGTTGATGAATGGCTACTCGACAAAGGTAAAAAACA | | | | | | Universal |
| 3801 | cg14988489 | GGAACACCATAAACGGGCTAAACGATGAAATTAACAAGCCATCTGAGGCG | | | | | | Universal |
| 3802 | cg15444935 | CGTCTTTGTTTGAAGGTCTTGGCAAACTGCTCAAGCTCCTCAAGGTCACT | | | | | | Universal |
| 3803 | cg16390944 | CGCTGATTGGCTGCCTGTTTGGATACATCTTTAGGGGAGCCTTGAAACTG | | | | | | Universal |
| 3804 | cg16545804 | GAAGGCACAATTTCCAGCAACACAATAAAATGTGCAGTGAATTTATGGCG | | | | | | Universal |
| 3805 | cg24713486 | CGGCTGCAGTGCACCAACTATCTTGGAGTGAATCTGAGCCCCAAGCTGGC | | | | | | Universal |
| 3806 | cg24856447 | CGACCATGAAAGGATTCAGCGTCTTGAATGAGACAGCTGCTTGTACAGAG | | | | | | Universal |
| 3807 | cg25034090 | CGGCCTTTGGGATTGTCAAGCTTAGTCAAGTCTCTTTGCAGCCGTGAGTT | | | | | | Universal |
| 3808 | cg26653463 | GGAACACTGAGACCTGTTGCCAATTTGAGTGAATATAGTGACTGTACTCG | | | | | | Universal |
| 3809 | cg25118671 | GGCCTCGCAGATTTTGATCTTAAGAAGGCCATTGAACACTACCATGGTCG | | | | | | Universal |
| 3810 | cg26922708 | CGTGAAGACTTAATTGCCTTATGGACATGGAAAGAATGGAGGGGCTTCTC | | | | | | Universal |
| 3811 | cg04533279 | CGCAGACATAGCCATAGTCGTAACCTGGGGACAGCATTTAAATACAGACG | | | | | | Universal |
| 3812 | cg11417778 | CGACAAGATGATATTAAAGATGAATCTTCGGAGTTTTCAAGCCATTCAAG | | | | | | Universal |
| 3813 | cg02042055 | GCTGTTTGATCGAGGTGTTCAAATGCTTTTAACCACCGTTGGTGCTTTCG | | | | | | Universal |
| 3814 | cg01990759 | AGATGGACGACAGCCATTGCTCGTCCTCCATGAAATTGCCGGGGGTCTCG | | | | | | Universal |
| 3815 | cg21871906 | TCCACCTTTGTAACCCCTGGTTTCCTCTTTTCCTGAATCACAAATTTACG | | | | | | Universal |
| 3816 | cg05736341 | CGGATGGGAACTGTCAGAGCCTGCCATCTGTGACAGCGCTTGGCATATTC | | | | | | Universal |
| 3817 | cg00817637 | GGGCCGCTGCTGGATAATTTGCAGTTTAATCAGCGTTTGTAATGAGAGCG | | | | | | Universal |
| 3818 | cg00900456 | TTCACAAGCCAGCAGTTGCAAGAGCTAGAGGCCACGTTCCAGAGGAACCG | | | | | | Universal |
| 3819 | cg01036016 | CGTGTGAAAATGAACAAACGCAAGCGTAGCACAGTTAATGAAAAGCCAAA | | | | | | Universal |
| 3820 | cg03225070 | GGGCTTCAGATATTTTGCACGAATTTTAAAACATGGCAATAAATGGCTCG | | | | | | Universal |
| 3821 | cg03856216 | CGGCTAAAGTCTCTCCAAGGTGACAAGCTGGAAGTCAGCTTGGTAAGTGT | | | | | | Universal |
| 3822 | cg04363039 | TTAAAAAAATCAACTAAACCATGCTTTTCAGAACAGAAGATTCATTATCG | | | | | | Universal |
| 3823 | cg06679388 | CGTCATCAGGCCTGGCAGAGCTCATTGGCAGAGAAGTTAAGAGTTTTATT | | | | | | Universal |
| 3824 | cg18974733 | CGTGCAGTTTGCTTCAAAGGAGATAGCAGAAAATGCTCTGGGGAAACACA | | | | | | Universal |
| 3825 | cg19275555 | CTTTAATGAAAACTGCAAGCACCAAAGGGTAACCATTTATTTATGAGTCG | | | | | | Universal |
| 3826 | cg11252613 | CGGCACATTTAACATGGTTAAAGCAGGGGAAAATAGTCATTAAGTTAATG | | | | | | Universal |
| 3827 | cg23869573 | CGGTATATACTAGCCTGCAATGAAGAAAGAACGAGACCCACATCATCAGC | | | | | | Universal |
| 3828 | cg05866252 | CAATCTCGCGGTGACTGATTAAGCCCTGTTTAGAACTAATTGCTCTTTCG | | | | | | Universal |
| 3829 | cg06244551 | CGTCGATACAATCACCAATGAGTCGATCCACAGGTACCGGGAACATAGAA | | | | | | Universal |
| 3830 | cg06413694 | TAGCTCCCTAATTAACTGTCTCAAATATATAATTACATTGCATCTTTACG | | | | | | Universal |
| 3831 | cg16764258 | ATGGTGGCGTACGCCCTTGTAATGATCAGCTCTGGTCGATGTGCTTACCG | | | | | | Universal |
| 3832 | cg25323457 | CGGCACTGCTATTTATAACTCATCTCAAATTGAGGGCAGACTGCCTAAAA | | | | | | Universal |
| 3833 | cg01772613 | CGCGAACTGTGCAAGCAAAGCCCCACATGGCTTTATCAGGAGTGTTGTGT | | | | | | Universal |
| 3834 | cg27245416 | GAGGTATTCATACTGGGCTGATACCCAGGGTTTGGAGTCATGGAATTCCG | | | | | | Universal |
| 3835 | cg01209550 | GTTAGCATTTTACGAGTTCTTTCCTCTACAGCTATAAAACTGCAGCTTCG | | | | | | Universal |
| 3836 | cg21883915 | CGGGAACGCTTACAGAGAGAGAGAGAGCGCCTAGAAATTGAAAGGCAAAA | | | | | | Universal |
| 3837 | cg02553697 | CGGATACTCAACAGTCCACTATCGGAGAGGGCTGTCAGATAAATCTTGTG | | | | | | Universal |
| 3838 | cg02658193 | TACCCGGAAGAGAACCCTTGGCCTTCTCAGCTGGGAGCTGGGAGCACACG | | | | | | Universal |
| 3839 | cg04577801 | GAACAAACATATAAATATATCTCTGCATTCCTGCCTCCCTCATAAAAGCG | | | | | | Universal |
| 3840 | cg22825729 | TGTAGTGCTGCGGTTTCTCGGGCTGTGCCTGCAGCACCTGAGCGGTAGCG | | | | | | Universal |
| 3841 | cg23059642 | AGGACATCATTGATGGATTTGCCATGACCAGCTTTGTCACTTTTGAAGCG | | | | | | Universal |
| 3842 | cg23126309 | ATCTTCCTAATTTTTCATCTCTTTAACAAACTCCTATGAAGTTGAAACCG | | | | | | Universal |
| 3843 | cg24638399 | TTCCTTTTGCCTCGTGCAATCCGTAATTGAAATGGAAAATCAAATGAACG | | | | | | Universal |
| 3844 | cg17417202 | CGCTTAGCAGCTATTATGTCGACTCGATCATAAGTCACGAGAGTGAGGAC | | | | | | Universal |
| 3845 | cg17542246 | CGCCTCTGCTGGAACAAGCAGGACCCTAACTACCTGGCCACCATGGCCAT | | | | | | Universal |
| 3846 | cg17554167 | CGATGTTGAATTTTAAAATCATGTGGATTATGGCGGATGGGGCCTGACAA | | | | | | Universal |
| 3847 | cg17942396 | AACAATTATTATTATAATTTTAAACAACCTGGTATTTTCCATTACTAACG | | | | | | Universal |
| 3848 | cg20453574 | CGGCAGGTTTAGTGGCATGGCAAACAATGCCTAACTCAGGCAAGTGTAAC | | | | | | Universal |
| 3849 | cg21482536 | AGCTGCTCTCGGCACAAGCATGTACGGCAAAGGCAAGAGTAACAGCAGCG | | | | | | Universal |
| 3850 | cg23320654 | CGCAGATAGCTGGGTTGCTTGATTTGAATTTGAGGAGTTGAAAAGCCTGT | | | | | | Universal |
| 3851 | cg07388493 | TCTTTCTCTCCTGTGACTTTGGTGAAGTCTCTCACCACTCAGTGTTGTCG | | | | | | Universal |
| 3852 | cg13294690 | AGCAATTCATCCCCGATCTCATCACAAAGTAACAGCGACCATCCTGCTCG | | | | | | Universal |
| 3853 | cg13943045 | CGTATAGTTGTACACTGGACAAATCTCCTGCAGAGCAACAAGATTTCCTA | | | | | | Universal |
| 3854 | cg15425194 | GGCCTGGGGAATAGAAACCCAAATCCACTTGTAATCGTACAAGAGATTCG | | | | | | Universal |
| 3855 | cg07987507 | CTTTTACAAACTAGTTGTTTTGTCAGCATGAGTGTGACAACAATTAGACG | | | | | | Universal |
| 3856 | cg18987683 | CGAGAAACTGGACAACCAACGGCTCAAGAATTTCAAGAACAAAGGCCGCG | | | | | | Universal |
| 3857 | cg08212459 | CGCCAGATTAGCCCCATCCATCCAGTGTCAATGGTTGTCTTGTTGAGAAA | | | | | | Universal |
| 3858 | cg08662919 | CGTGGGGTGGTTCCTATTTCTGTCGGCAAATACAAATTTTTCACTCAGCA | | | | | | Universal |
| 3859 | cg04080945 | CGGCACTCAGTACATTAATTCACGCTGAAGTCCGGTAAATCAAAATTATA | | | | | | Universal |
| 3860 | cg03626038 | CATTGTCCAAGTGGAGTGCCATTTCTTCTGATATGAAAGTGTTCAAATCG | | | | | | Universal |
| 3861 | cg06197482 | TTCTTAGCCCCGCTCCCATGTGAGGCCTAAACAAAGACTTTGGGGAGACG | | | | | | Universal |
| 3862 | cg06459870 | CGTCGTTGGCTAGGTTTTGGATTTATTACTTTCGAGGACGAACAATCAGT | | | | | | Universal |
| 3863 | cg20140365 | CGAGACATTCAGTGCTTTTTAAATTGGAAGTTGCATTGTGCTAAAGACCT | | | | | | Universal |
| 3864 | cg22063749 | GCATTTTAGCTAAACTAAGACTTGCCCTGCATGGTGCCAGCTGTAGCACG | | | | | | Universal |
| 3865 | cg22414596 | ATTAATGGCTTTTCTCAGCGGAATTGTAAATTCAAATGTCACCTCACACG | | | | | | Universal |
| 3866 | cg22667099 | CTATCTCTTGTTCTGAATAGAACCAATTTTCAGAGAGCTATGAAATCACG | | | | | | Universal |
| 3867 | cg01759800 | CGGAGAGCAGGGAAGGCTAAAGCCGGAGCCTAATTCATGATGCATGAACG | | | | | | Universal |
| 3868 | cg18221275 | GTTTGTCATCGTACCAATGCATGCAAAGCATTCCACTCCCAGAAACAACG | | | | | | Universal |
| 3869 | cg13581054 | CGGCACATTAGGCAGTGCGCTTATTTTGGGCTCCGCTTCAGTGGATTAAC | | | | | | Universal |
| 3870 | cg13705955 | TGAGGCCAGACAGTAGAGGCAGGTTATAGATCACAGCACATGGAGAAGCG | | | | | | Universal |
| 3871 | cg14258341 | TTGACTCCTTTCAGTTGATAATTCATGGCTTGCATATGCGCTTTAATGCG | | | | | | Universal |
| 3872 | cg00292639 | ACACACAGCAGGAAGGCAAACATCAGCTTGTGCTTCTCAAAGAGGCTGCG | | | | | | Universal |
| 3873 | cg15546160 | CGGAAATTGCATAGTAGACCTCGTAAACTTTATAAACATTCAAGGTAGGT | | | | | | Universal |
| 3874 | cg12967137 | CGGGGTGCACCGAGCTGTCGGGTTAAACAAACAACTTGTACCCTGACCAA | | | | | | Universal |
| 3875 | cg13313332 | GCTACAGTAAGTTATTCCAATTTTTTTTTAACATTTTTCCTGACTTTCCG | | | | | | Universal |
| 3876 | cg14006844 | CGGCTTCATCCTGCGCCAGTTCAACGCCTGCCCCTGGGACTACTCCCAGT | | | | | | Universal |
| 3877 | cg04228573 | CGCCCTTCGCAGGAAGGACGTCTGCGCACTAAGATACTCAGTTCCAAGTT | | | | | | Universal |
| 3878 | cg04404159 | CGAAAGTTAAGTCTTCGTGGATGTCTTGGAGTGGGAGACAATGCATTAAG | | | | | | Universal |
| 3879 | cg06493080 | CTCTAATTCTGTAATATATCAAGGAATCTCGTAAAACCGACACTAAAACG | | | | | | Universal |
| 3880 | cg09161688 | CGTGTTTGGAATTGTTGTGCACAAACATGTTGTCTGACACGGCCAGCACG | | | | | | Universal |

### CONCLUSION

This concludes the description of the preferred embodiment of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

1. A method for obtaining information associated with an age of a mammal, the method comprising:
obtaining genomic DNA from the mammal;
observing CpG methylation of the genomic DNA in a group of at least 40 methylation markers present in genomic polynucleotides having SEQ ID NO: 1-SEQ ID NO: 3880; and
correlating methylation observed in the methylation markers with an age of the mammal; so that information associated with an age of the mammal is obtained.

2. The method of claim 1, wherein:
methylation of the genomic DNA is observed in a plurality of methylation markers present in in polynucleotides having SEQ ID NO: 1-SEQ ID NO: 956 such that the methylation markers observed are selected to be methylation markers whose methylation status is associated with an age in both humans and dogs.

3. The method of claim 1, wherein:
methylation of the genomic DNA is observed in a plurality of methylation markers present in in polynucleotides having SEQ ID NO: 2220-SEQ ID NO: 3043 such that methylation markers observed are selected to be methylation markers whose methylation status is associated with an age in both humans and rats.

4. The method of claim 1, wherein:
methylation of the genomic DNA is observed in a plurality of methylation markers present in in polynucleotides having SEQ ID NO: 1222-SEQ ID NO: 2219 such that methylation markers observed are selected to be methylation markers whose methylation status is associated with an age in both humans and mice.

5. The method of claim 1, wherein:
methylation of the genomic DNA is observed in a plurality of methylation markers present in in polynucleotides having SEQ ID NO: 3044-SEQ ID NO: 3880 such that methylation markers observed are selected to be methylation markers whose methylation status is associated with an age in a plurality of mammalian species.

6. The method of claim 1, wherein the method comprises correlating methylation observed in the methylation markers with an epigenetic age and/or a chronological age of the mammal.

7. The method of claim 6, wherein the method further compares an epigenetic age obtained for the sample with the chronological age of the mammal.

8. The method of claim 1, wherein the method comprises determining an epigenetic age of the biological sample with a statistical prediction algorithm, comprising (a) obtaining a linear combination of the methylation marker levels, and (b) applying a transformation to the linear combination to determine an epigenetic age of the biological sample.

9. The method of claim 1, wherein:
methylation is observed by a process comprising treatment of genomic DNA from the population of cells from the individual with bisulfite to transform unmethylated cytosines of CpG dinucleotides in the genomic DNA to uracil;
genomic DNA is obtained from fibroblasts, keratinocytes, buccal cells, endothelial cells, lymphoblastoid cells, and/or cells obtained from blood, skin, dermis, epidermis or saliva;
genomic DNA is hybridized to a complimentary sequence disposed on a microarray; and/or
correlating observed methylation in the methylation markers comprises a regression analysis.

10. A method of observing the effects of an environmental condition on genomic methylation associated epigenetic aging of mammalian cells, the method comprising:
(a) exposing mammalian cells to the environmental condition;
(b) observing methylation status in at least 40 of the methylation markers present in polynucleotides having SEQ ID NO: 1-SEQ ID NO: 3880 in genomic DNA from the mammalian cells;
(c) comparing the observations from (b) with observations of a methylation status at least 40 of the methylation markers present in in polynucleotides having SEQ ID NO: 1-SEQ ID NO: 3880 in genomic DNA from control mammalian cells not exposed to the environmental condition such that effects of the environmental condition on genomic methylation associated epigenetic aging in the mammalian cells is observed.

11. The method of claim 10, wherein:
the plurality of the methylation markers observed are selected to be methylation markers whose methylation status is associated with age in both humans and dogs, and/or
the cells are human and/or dog cells.

12. The method of claim 10, wherein:
the plurality of the methylation markers observed are selected to be methylation markers whose methylation status is associated with age in both humans and rats; and/or
the cells are human and/or rat cells.

13. The method of claim 10, wherein:
the plurality of the methylation markers observed are selected to be methylation markers whose methylation status is associated with age in both humans and mice; and/or
the cells are human and/or mouse cells.

14. The method of claim 10, wherein:
a plurality of the methylation markers observed are selected to be methylation markers whose methylation status is associated with an age in a plurality of mammalian species and
the cells are human cells.

15. The method of claim 10, wherein:
methylation is observed by a process comprising treatment of genomic DNA from the population of cells from the individual with bisulfite to transform unmethylated cytosines of CpG dinucleotides in the genomic DNA to uracil;
genomic DNA is obtained from fibroblasts, keratinocytes, buccal cells, endothelial cells, lymphoblastoid cells, and/or cells obtained from blood, skin, dermis, epidermis or saliva;
genomic DNA is hybridized to a complimentary sequence disposed on a microarray; and/or
correlating observed methylation in the methylation markers comprises a regression analysis.

16. The method of claim 10, wherein the environmental condition comprises exposure to a composition of matter.

17. The method of claim 16, wherein the composition of matter is combined with mammalian cells for at least 1 day, at least 1 week or at least 1 month.

18. The method of claim 17, wherein the composition of matter comprises a test agent having a molecular weight of <900 Da.

19. A tangible computer-readable medium comprising computer-readable code that, when executed by a computer, causes the computer to perform operations comprising:
a) receiving information corresponding to a methylation status of a set of methylation markers in a biological sample, said methylation markers comprising methylation markers present in genomic polynucleotides having SEQ ID NO: 1-SEQ ID NO: 3880; and
b) determining an age of the biological sample by applying a statistical prediction algorithm to the measured methylation marker levels.

20. The tangible computer-readable medium of claim 19, further comprising computer-readable code that, when executed by a computer, causes the computer to perform one or more additional operations comprising: sending information corresponding to the methylation levels of the set of methylation markers in the biological sample to a tangible data storage device.
